(19) United States
(12) Patent Application Publication
(10) Pub. No.: US 2025/0177354 A1
(43) Pub. Date: Jun. 5, 2025

(54) THERAPEUTIC COMBINATIONS, COMPOSITIONS, AND METHODS FOR DESIGNING AND PRODUCING ENTACTOGENIC MINDSTATES

(71) Applicant: Mindstate Design Labs, Inc., Canonsburg, PA (US)

(72) Inventor: Thomas Ray, Norman, OK (US)

(21) Appl. No.: 18/845,088

(22) PCT Filed: Mar. 9, 2023
(86) PCT No.: PCT/US2023/014926

§ 371 Date: Sep. 9, 2024

Related U.S. Application Data
(60) Provisional application No. 63/318,369, filed on Mar. 9, 2022.

(51) Int. Cl. A61K 31/4168; A61K 9/00; A61K 31/00; A61K 31/4045; A61K 31/421; A61K 31/506; A61P 25/00

(52) U.S. Cl. CPC A61K 31/4168; A61K 9/0019; A61K 31/4045; A61K 31/421; A61K 31/506; A61K 31/658; A61P 25/00

(57) ABSTRACT

Disclosed are therapeutic combinations for eliciting an entactogenic mindstate, such as through use of compositions comprising imidazoline receptor agents, cannabinoid receptor agents, and/or serotonin receptor agents. Also disclosed are certain selective receptor agents, pharmaceutical compositions thereof, and methods for their use to treat mental health conditions, improve mental health and mental functioning, and design and create entactogenic mental states or mindstates.

### CROSS-REFERENCE

Priority is claimed under PCT Article 8(1) and Rule 4.10 to U.S. Prov. Appl. No. 63/318,369 filed Mar. 9, 2022 and incorporated by reference for all purposes as if fully set forth herein.

### FIELD OF THE INVENTION

This disclosure relates to the creation of entactogenic mental states through the use of therapeutic combinations, such as compositions of imidazoline receptor agents, cannabinoid receptor agents, and/or serotonin receptor agents.

### BACKGROUND OF THE INVENTION

3,4-methylenedioxymethamphetamine (MDMA) is a member of a class of drugs called empathogens, or entactogens, that are a promising therapeutic option for mental health disorders such as PTSD (Mitchell, 2021). Clinical studies have demonstrated that MDMA, when taken in combination with psychotherapy, has rapid and long-lasting therapeutic effects.

MDMA is also known to have numerous adverse physiological effects, making its use contraindicated in certain populations (Oeri, 2020). MDMA and its metabolites also may pose safety risks associated with toxicity (Kalant, 2001). Further, MDMA has tolerance-inducing effects that cause many to suffer a partial or complete loss of the entactogenic effects (“loss of magic”), hindering repeated use.

Accordingly, there is a need for treatment options that provide the entactogenic effects of MDMA upon administration, as well as the enduring therapeutic benefits, without the adverse effects and health risks associated with it and other entactogens. There is a need for such treatment options for mental health conditions, for the betterment of health and functioning generally, and more broadly for human flourishing. Disclosed herein are therapeutic combinations, pharmaceutical compositions, and methods of their use to meet these needs, which have such other advantages as will become apparent from the disclosure below.

### INCORPORATION BY REFERENCE

Each patent, publication, and non-patent literature cited or listed in the “References” section is incorporated by reference in its entirety as if it was incorporated by reference individually and set forth fully herein. However, where such reference is made, and whether to patents, publications, non-patent literature, or other sources of information, it is for the general purpose of providing context for discussing features of the disclosed invention. Accordingly, it should not be construed as an admission that the document or information, in any jurisdiction, is prior art, or forms part of the common general knowledge in the art.

### BRIEF SUMMARY OF THE INVENTION

The following presents a simplified summary of some embodiments of the invention in order to provide a basic understanding thereof. It is not a comprehensive overview of the disclosure, nor intended to identify key or critical elements of the disclosure or to delineate its scope. Its sole purpose is to present some embodiments in a simplified form as a prelude to the more detailed description that is presented later.

In a first aspect, provided is a therapeutic combination for eliciting an entactogenic mindstate, comprising an imidazoline-1 (I₁) agent and a 5-HT₇ agent.

In some embodiments, the I₁ agent is an I₁ agonist. In some embodiments, the I₁ agent has a selectivity for the I₁ receptor over the alpha-2 (α2) adrenergic receptor of at least about 30:1. In some embodiments, the I₁ agent is agmatine, BDBM50091347, clonidine, guanfacine, mCPP, memantine, moxonidine, naphazoline, oxymetazoline, rilmenidine, tetryzoline, tizanidine, or tolonidine, or a pharmaceutically acceptable salt thereof. In some embodiments, the I₁ agent is clonidine, moxonidine, or rilmenidine, or a pharmaceutically acceptable salt thereof. In some embodiments, the I₁ agent is clonidine, or a pharmaceutically acceptable salt thereof. In some embodiments, the I₁ agent is moxonidine, or a pharmaceutically acceptable salt thereof. In some embodiments, the I₁ agent is rilmenidine, or a pharmaceutically acceptable salt thereof.

In some embodiments, the 5-HT₇ agent is a 5-HT₇ agonist. In some embodiments, the 5-HT₇ agent has a 5-HT₇ receptor Kᵢ of less than 10 nM. In some embodiments, the 5-HT₇ agent is a tryptamine. In some embodiments, the 5-HT₇ agent is 5-MeO-DMT or 5-MeO-MiPT, or a pharmaceutically acceptable salt thereof. In some embodiments, the 5-HT₇ agent is 5-MeO-DMT, or a pharmaceutically acceptable salt thereof. In embodiments, the 5-HT₇ agent is 5-MeO-MiPT, or a pharmaceutically acceptable salt thereof.

In some embodiments, the therapeutic combination comprises clonidine, or a pharmaceutically acceptable salt thereof, and 5-MeO-DMT, or a pharmaceutically acceptable salt thereof. In some embodiments, the therapeutic combination comprises moxonidine, or a pharmaceutically acceptable salt thereof, and 5-MeO-DMT, or a pharmaceutically acceptable salt thereof. In some embodiments, the therapeutic combination comprises rilmenidine, or a pharmaceutically acceptable salt thereof, and 5-MeO-DMT, or a pharmaceutically acceptable salt thereof.

In some embodiments, the therapeutic combination comprises clonidine, or a pharmaceutically acceptable salt thereof, and 5-MeO-MiPT, or a pharmaceutically acceptable salt thereof. In some embodiments, the therapeutic combination comprises moxonidine, or a pharmaceutically acceptable salt thereof, and 5-MeO-MiPT, or a pharmaceutically acceptable salt thereof. In some embodiments, the therapeutic combination comprises rilmenidine, or a pharmaceutically acceptable salt thereof, and 5-MeO-MiPT, or a pharmaceutically acceptable salt thereof.

In some embodiments, the therapeutic combination further comprises a 5-HT₂ agent, a CB₁ agent, an additional I₁ agent, or an additional 5-HT₇ agent.

In some embodiments, the therapeutic combination further comprises a 5-HT₂ agent. In some embodiments, the 5-HT₂ agent is a 5-HT₂ agonist. In some embodiments, the 5-HT₂ agonist is 1-methylpsilocin, 2C-B-FLY, 5-MeO-AMT, AL-37350A, CP 809101, DALT, DOB, DOET, DOHx, MEM, CPP, NBOH-2C-CN, TCB-2, or WAY 161503, or a pharmaceutically acceptable salt thereof.

In some embodiments, the therapeutic combination further comprises a CB₁ agent. In some embodiments, the CB₁ agent is a cannabinoid. In some embodiments, the CB₁ agent is 2-AGE, A-834,735, ACEA, AZ-11713908, AZD-1940, CBN, CP-47497, CP55940, HU-210, JWH-007, JWH-051, JWH-146, JWH-176, JWH-200, JWH-398, Org 28611, rimonabant, THC, WIN 55,212-2, XLR11, or a pharmaceutically acceptable salt thereof.

In some embodiments, the therapeutic combination further comprises an additional I₁ agent. In some embodiments, the additional I₁ agent is an I₁ agonist. In some embodiments, the additional I₁ agent has a selectivity for the I₁ receptor over the alpha-2 (α2) adrenergic receptor of at least about 30:1. In some embodiments, the additional I₁ agent is agmatine, BDBM50091347, clonidine, guanfacine, mCPP, MDMA, memantine, moxonidine, naphazoline, oxymetazoline, rilmenidine, tetryzoline, tizanidine, or tolonidine, or a pharmaceutically acceptable salt thereof. In some embodiments, the additional I₁ agent is clonidine, moxonidine, or rilmenidine, or a pharmaceutically acceptable salt thereof.

In some embodiments, the therapeutic combination further comprises an additional 5-HT₇ agent. In embodiments, the additional 5-HT₇ agent is a 5-HT₇ agonist. In embodiments, the additional 5-HT₇ agent has a 5-HT₇ receptor Kᵢ of less than 10 nM. In embodiments, the additional 5-HT₇ agent is DMT, 6-F-DMT, 5-MeO-MiPT, LSD, 5-MeO-DMT, 5-MeO-TMT, cis-2a, DPT, 5-MeO-DiPT, psilocin, RR-2b, EMDT, lisuride, DiPT, SS-2c, TMA, 2C-B, 2C-E, or MDA, or pharmaceutically acceptable salt thereof. In embodiments, the additional 5-HT₇ agent is 5-MeO-DMT or 5-MeO-MiPT, or a pharmaceutically acceptable salt thereof.

In some embodiments, the therapeutic combination further comprises: (a) a 5-HT₂ agent and a CB₁ agent; (b) a 5-HT₂ agent and an additional I₁ agent; (c) a 5-HT₂ agent and an additional 5-HT₇ agent; (d) a 5-HT₂ agent and an additional 5-HT₂ agent; (e) a CB₁ agent and an additional I₁ agent; (f) a CB₁ agent and an additional 5-HT₇ agent; or (g) a CB₁ agent and an additional CB₁ agent.

In some embodiments wherein the therapeutic combination further comprises (a) a 5-HT₂ agent and a CB₁ agent; (b) a 5-HT₂ agent and an additional I₁ agent; (c) a 5-HT₂ agent and an additional 5-HT₇ agent; or (d) a 5-HT₂ agent and an additional 5-HT₂ agent; the 5-HT₂ agent is a 5-HT₂ agonist. In embodiments, the 5-HT₂ agonist is 1-methylpsilocin, 2C-B-FLY, 5-MeO-AMT, AL-37350A, CP 809101, DALT, DOB, DOET, DOHx, MEM, CPP, NBOH-2C-CN, TCB-2, or WAY 161503, or a pharmaceutically acceptable salt thereof.

In some embodiments wherein the therapeutic combination further comprises (e) a CB₁ agent and an additional I₁ agent; (f) a CB₁ agent and an additional 5-HT₇ agent; or (g) a CB₁ agent and an additional CB₁ agent; the CB₁ agent is a cannabinoid. In some embodiments, the CB₁ agent is 2-AGE, A-834,735, ACEA, AZ-11713908, AZD-1940, CBN, CP-47497, CP55940, HU-210, JWH-007, JWH-051, JWH-146, JWH-176, JWH-200, JWH-398, Org 28611, rimonabant, THC, WIN 55,212-2, XLR11, or a pharmaceutically acceptable salt thereof. In some embodiments, the additional CB₁ agent is a cannabinoid. In some embodiments, the additional CB₁ agent is 2-AGE, A-834,735, ACEA, AZ-11713908, AZD-1940, CBN, CP-47497, CP55940, HU-210, JWH-007, JWH-051, JWH-146, JWH-176, JWH-200, JWH-398, Org 28611, rimonabant, THC, WIN 55,212-2, XLR11, or a pharmaceutically acceptable salt thereof.

In some embodiments wherein the therapeutic combination further comprises (b) a 5-HT₂ agent and an additional I₁ agent or (e) a CB₁ agent and an additional I₁ agent; the additional I₁ agent is an I₁ agonist. In embodiments, the additional I₁ agent has a selectivity for the I₁ receptor over the alpha-2 (α2) adrenergic receptor of at least about 30:1. In embodiments, the additional I₁ agent is agmatine, BDBM50091347, clonidine, guanfacine, mCPP, memantine, moxonidine, naphazoline, oxymetazoline, rilmenidine, tetryzoline, tizanidine, or tolonidine, or a pharmaceutically acceptable salt thereof. In embodiments, the additional I₁ agent is clonidine, moxonidine, or rilmenidine, or a pharmaceutically acceptable salt thereof. In embodiments, the additional I₁ agent is clonidine, or a pharmaceutically acceptable salt thereof. In embodiments, the additional I₁ agent is moxonidine, or a pharmaceutically acceptable salt thereof. In embodiments, the additional I₁ agent is rilmenidine, or a pharmaceutically acceptable salt thereof.

In some embodiments wherein the therapeutic combination further comprises (c) a 5-HT₂ agent and an additional 5-HT₇ agent or (f) a CB₁ agent and an additional 5-HT₇ agent, the additional 5-HT₇ agent has a 5-HT₇ receptor Kᵢ of less than 10 nM. In some embodiments, the additional 5-HT₇ agent is DMT, 6-F-DMT, 5-MeO-MiPT, LSD, 5-MeO-DMT, 5-MeO-TMT, cis-2a, DPT, 5-MeO-DiPT, psilocin, RR-2b, EMDT, lisuride, DiPT, SS-2c, TMA, 2C-B, 2C-E, or MDA, or pharmaceutically acceptable salt thereof. In embodiments, the additional 5-HT₇ agent is 5-MeO-DMT or 5-MeO-MiPT, or a pharmaceutically acceptable salt thereof.

In another aspect, provided is a pharmaceutical composition comprising a therapeutic combination of the disclosure, and a pharmaceutically acceptable carrier, diluent, or excipient. In some embodiments, the composition is suitable for oral, buccal, sublingual, intranasal, ophthalmic, injectable, subcutaneous, intravenous, or transdermal administration. In some embodiments, the composition is provided in unit dosage form. In some embodiments, said unit dosage form is an immediate release, controlled release, sustained release, extended release, or modified release formulation.

In another aspect, provided is a method of eliciting an entactogenic mindstate in a subject, comprising administering to the subject the therapeutic combination or the pharmaceutical composition of any of the preceding embodiments.

In some embodiments, the agents of the therapeutic combination or the pharmaceutical composition are administered simultaneously, separately, or sequentially. In some embodiments, the agents are administered simultaneously. In some embodiments, the agents are administered separately. In some embodiments, the agents are administered sequentially. In some embodiments, the I₁ agent is administered prior to the 5-HT₇ agent. In embodiments wherein the therapeutic combination or composition comprises a CB₁ agent, the CB₁ agent is administered prior to the 5-HT₇ agent. In embodiments wherein the therapeutic combination or composition comprises a CB₁ agent, the CB₁ agent is administered after the I₁ agent.

In some embodiments, the I₁ agent is administered orally. In some embodiments, the 5-HT₇ agent is administered orally, sublingually, intranasally, or by inhalation. In some embodiments wherein the therapeutic combination or composition comprises a CB₁ agent, the CB₁ agent is administered orally, sublingually, or by inhalation.

In some embodiments, the I₁ agent is administered at a dose of between about 0.1 mg and 1.0 mg.

In embodiments, the 5-HT₇ agent is administered at a dose of between about 2 mg and 20 mg.

In some embodiments, the method further comprises administering a therapeutically effective amount of an additional active agent. In some embodiments, the additional therapeutic agent is an amino acid, antioxidant, anti-inflammatory agent, analgesic, antineuropathic or antinociceptive agent, antimigraine agent, anxiolytic, antidepressant, antipsychotic, anti-PTSD agent, cannabinoid, dissociative, immunostimulant, anti-cancer agent, antiemetic, orexigenic, antiulcer agent, antihistamine, anti hypertensive, anticonvulsant, antiepileptic, bronchodilator, neuroprotectant, entactogen or empathogen, entheogen, psychedelic, monoamine oxidase inhibitor, tryptamine, terpene, phenethylamine, sedative, serotonergic agent, stimulant, vitamin, SSRI, SNRI, NDRI, TCA, or benzodiazepine.

In some embodiments, the entactogenic mindstate is a similar entactogenic mindstate to MDMA or another known entactogen, wherein the entactogenic mindstate is determined by brain imaging or by measuring the level of one or more neurotransmitters in the subject. In some embodiments, the entactogenic mindstate is a greater entactogenic mindstate to MDMA or another known entactogen, wherein the entactogenic mindstate is determined by brain imaging or by measuring the level of one or more neurotransmitters in the subject. In some embodiments, the greater entactogenic mindstate is an expanded entactogenic mindstate or an enhanced entactogenic mindstate, when compared to MDMA or another known entactogen, wherein the entactogenic mindstate is determined by brain imaging or by measuring the level of one or more neurotransmitters in the subject. In some embodiments, the neurotransmitter is one or more of serotonin, dopamine, norepinephrine, oxytocin, or cortisol.

In some embodiments, the method does not produce an adverse effect of MDMA or another known entactogen, or produces a reduced adverse effect of MDMA or another known entactogen. In some embodiments, the adverse effect is a neurotoxic effect. In some embodiments, an absence or reduction of a neurotoxic effect is determined by tests and procedures that are in silico, in vitro, or in vivo. In some embodiments, the absence or reduction of a neurotoxic effect is determined by measuring one or more of: a) at least one toxic metabolite of MDMA or at least one toxic metabolite of another entactogen; b) oxidative stress and dopamine-based quinones; c) mitochondrial dysfunction; and d) activation of glial cells. In some embodiments, the reduction of a neurotoxic effect is at least 5%, 10%, 25%, 50%, 75%, 100%, 150%, or 200% relative to one or more comparators.

Also provided is a method for modulating neurotransmission in a mammal, comprising administering to the mammal the therapeutic combination or the pharmaceutical composition of any of the preceding embodiments. In some embodiments, the neurotransmission is mediated by an I₁ receptor, a 5-HT₂ receptor, a 5-HT₇ receptor, or a CB₁ receptor. In some embodiments, the mammal is a human.

In another aspect, provided is a method of treating a medical condition in a human in need of such treatment, comprising administering to the human the therapeutic combination or the pharmaceutical composition of any of the preceding embodiments.

In some embodiments, the medical condition is a disorder linked to dysregulation or inadequate functioning of neurotransmission. In some embodiments, the disorder is linked to dysregulation or inadequate functioning of neurotransmission mediated by an I₁ receptor, a 5-HT₂ receptor, a 5-HT₇ receptor, or a CB₁ receptor.

In some embodiments, the medical condition is a CNS disorder. In some embodiments, the CNS disorder is any of: post-traumatic stress disorder (PTSD), adjustment disorder, affective disorder, depression, atypical depression, postpartum depression, catatonic depression, a depressive disorder due to a medical condition, premenstrual dysphoric disorder, seasonal affective disorder, dysthymia, anxiety, phobia disorders, binge disorders, body dysmorphic disorder, alcohol or drug abuse or dependence disorders, substance-related disorders, substance-induced mood disorder, a mood disorder related to another health condition, disruptive behavior disorders, eating disorders, impulse control disorders, obsessive compulsive disorder (OCD), attention deficit hyperactivity disorder (ADHD), personality disorders, attachment disorders, dissociative disorders, Alzheimer's disease, ataxia, Huntington's disease, Parkinson's disease, motor neuron disease, multiple system atrophy, progressive supranuclear palsy, migraines, cluster headaches, short-lasting unilateral neuralgiform headaches, fibromyalgia, traumatic brain injury, and mild traumatic brain injury (mTBI). In some embodiments, the CNS disorder is a disorder related to rigid modes of thinking. In some embodiments, the disorder related to rigid modes of thinking is anxiety, depression, addiction, an eating disorder, an alcohol or drug abuse or dependence disorder, OCD, or PTSD. In some embodiments, depression is MDD or TRD. In some embodiments, anxiety is GAD.

In another aspect, method of improving mental health or functioning in a human, the method comprising identifying a human in need of said improving, and administering to the human the therapeutic combination or the pharmaceutical composition of any of the preceding embodiments. In some embodiments, the improvement in mental health or functioning is a reduction of neuroticism or psychological defensiveness, an increase in creativity or openness to experience, an increase in decision-making ability, an increase in ability to fall or stay asleep, an increase in feelings of wellness or satisfaction, an increase in the ability to forgive oneself or another, an increase in the ability to experience or contemplate pain, including mental pain, or an increase in the ability to “touch within,” including in and during everyday life and activities. In some embodiments, the increase in creativity is an increase in deliberate cognitive creativity, deliberate emotional creativity, spontaneous cognitive creativity, or spontaneous emotional creativity.

In another aspect, provided is a method of reducing the symptoms of a CNS disorder in a human, the method comprising identifying a human in need of said reducing, and administering to the human a therapeutic combination or a pharmaceutical composition of the disclosure.

In another aspect, provided is a pharmaceutical composition comprising the therapeutic combination of any one of the preceding embodiments, and a pharmaceutically acceptable carrier, diluent, or excipient. In some embodiments, the composition is suitable for oral, buccal, sublingual, intranasal, ophthalmic, injectable, subcutaneous, intravenous, or transdermal administration. In some embodiments, the composition is provided in unit dosage form. In embodiments, said unit dosage form is an immediate release, controlled release, sustained release, extended release, or modified release formulation.

In another aspect, provided is a method of eliciting an entactogenic mindstate in a subject, comprising administering to the subject a therapeutic combination or a pharmaceutical composition of the disclosure.

In another aspect, provided is a method for modulating neurotransmission in a mammal, comprising administering to the mammal the therapeutic combination or pharmaceutical composition of the disclosure.

In another aspect, provided is a method of treating a medical condition in a human in need of such treatment, comprising administering to the human a therapeutic combination or a pharmaceutical composition of the disclosure.

In another aspect, provided is a method of improving mental health or functioning in a human, the method comprising identifying a human in need of said improving, and administering to the human a therapeutic combination or a pharmaceutical composition of the disclosure.

In another aspect, provided is a method of reducing the symptoms of a CNS disorder in a human, the method comprising identifying a human in need of said reducing, and administering to the human a therapeutic combination or a pharmaceutical composition of the disclosure.

In another aspect, provided is a therapeutic combination for eliciting an entactogenic mindstate, comprising: (a) the therapeutic combination of any one of claims 1-53; or (b) an I₁ agent and one or more of a 5-HT₂ agent, a 5-HT₇ agent, and a CB₁ agent.

Also provided in further aspects is the method of any of the foregoing embodiments, wherein the pharmaceutical composition or therapeutic combination is administered together with psychotherapy. Also provided is the method of any of the foregoing embodiments, wherein the pharmaceutical composition or therapeutic combination is administered together with psychological support. Also provided is the method of any of the foregoing embodiments, wherein the pharmaceutical composition or therapeutic combination is administered together with patient monitoring. Also provided is the method of any of the foregoing embodiments, wherein the pharmaceutical composition or therapeutic combination is administered together with the performance of a therapeutically beneficial activity by the patient. Also provided is the method of any of the foregoing embodiments, wherein the pharmaceutical composition or therapeutic combination is administered without psychotherapy or psychological support.

The foregoing has outlined broadly and in summary certain pertinent features of the disclosure so that the detailed description of the invention that follows may be better understood, and so the contribution to the art can be more fully appreciated. It is to be considered as a brief and general synopsis of only some of the disclosed aspects and embodiments, is solely for the benefit and convenience of the reader, and is not intended to limit in any manner the scope, or range of equivalents, to which the claims are entitled. Additional features of the invention are described below. It will be appreciated by those in the art that all disclosed specific compositions and methods are only exemplary, and may be readily utilized as a basis for modifying or designing other compositions and methods for carrying out the same purposes. Such equivalent compositions and methods will be appreciated to be also within the scope and spirit of the disclosure as recited in the claims. It will be appreciated that headings within this document are being utilized only to expedite its review. They should not be construed as limiting the invention in any manner.

### DETAILED DESCRIPTION OF THE INVENTION

While certain aspects and embodiments are summarized above, the following description illustrates several exemplary embodiments in further detail to enable one of skill in the art to practice such embodiments, and to make and use the full scope of the invention claimed. The described examples are provided for illustrative purposes and are not intended to limit the scope of the invention or its applications. It will be understood that many modifications, substitutions, changes, and variations in the described examples, embodiments, applications, and details of the invention illustrated herein can be made by those in the art without departing from the spirit of the invention, or the scope of the invention as described in the appended claims. It also will be appreciated that the headings within this document are being utilized only to expedite its review by a reader. They should not be construed as limiting the invention in any manner.

MDMA, and other known entactogens, are known to be “messy” drugs from a pharmacological perspective, having a broad spectrum of separate and independent mechanisms (Ray, 2016; Sessa, 2017).

MDMA, for example, is believed to exert its effects by promoting raised levels of serotonin (5-HT), dopamine (DA), and norepinephrine (NE), increasing activity at 5-HT_{1A} and 5-HT_{1B} receptors (Graeff et al., 1996), acting at 5-HT₂ receptors, in common with “classic” psychedelics (Liechti and Vollenweider, 2001), acting at alpha-2 receptors (Bexis and Docherty, 2005; Giovannitti et al., 2015), and facilitating release of oxytocin (Thompson et al., 2007; Kirsch et al., 2005; Domes et al., 2007). Prior work by the inventor demonstrates that MDMA acts on at least imidazoline-1 receptors; serotonin-2B receptors; alpha-2A, alpha-2B, and alpha-2C adrenergic receptors; muscarinic-3, muscarinic-4, and muscarinic-5 acetylcholine receptors; and Ca²⁺ channels (Ray, 2010). A more recent study reported the binding affinity profile of MDMA across much of the receptorome (see Table 5 and accompanying text in Dunlap, 2018).

The broader class of phenylalkylamine and tryptamine entactogens generally includes compounds known to be potent releasers and/or reuptake inhibitors of presynaptic 5-HT, DA, and NE, actions which result from their interaction with the membrane transporters involved in neurotransmitter reuptake and vesicular storage systems (e.g., SERT, DAT, NET, VMAT) (de la Torre 2004); to have direct effects on a variety of receptors, including (among others), 5-HT_{1A}, 5-HT_{1D}, 5-HT_{1E}, 5-HT_{2A}, 5-HT_{5A}, 5-HT₆, 5-HT₇, D₁, D₂, D₃, D₄, D₅, NMDA, and imidazoline-1 (Ray 2010; Vegting 2016); and to be 5-HT releasers by a Ca²⁺-independent mechanism. They also can inhibit 5-HT reuptake and inhibit monoamine oxidase (MAO), both of which can result in an increased amount of extracellular 5-HT (Leonardi 1994; Simmler 2018).

This “messy” pharmacology is believed to be responsible for the adverse effects of entactogens. However, agents with more selective activity, and consequently lacking one or more adverse effects, also do not provide a full complement of entactogenic effects necessary to provide an entactogenic mindstate, nor may they consequently provide enduring therapeutic benefits. It has not been possible to decouple the many adverse effects of entactogens from their “entactogenic” effects. That is, until the work of this inventor.

In aspects of this disclosure, separate receptor systems are independently targeted, enabling the creation of combinations or compositions with a specific effect profile, where such effect profile is a desired mindstate, in particular an entactogenic mindstate. In one example, the entactogenic effects of MDMA are recreated by a disclosed combination or composition, which provides the same (or in some preferred embodiments, greater) therapeutic benefits of MDMA without its inherent drawbacks.

In some embodiments, the disclosed combinations and compositions may be used in entactogen-assisted therapy (EAP), including EAP with individuals, couples, and groups, without one or more adverse effects or complications seen with prior art entactogens like MDMA.

In some embodiments, where MDMA itself is used in a disclosed combination or composition, the combination will result in a substantially decreased dose of MDMA necessary to elicit an entactogenic mindstate, thus decreasing the risk of adverse side effects compared to the administration of MDMA alone.

In some aspects, the entactogenic effects of MDMA or another entactogen is obtained through selective and simultaneous, separate, or sequential activation of specific receptor systems by coadministration (including simultaneous, separate, or sequential administration) of a “primer” and a “probe.” In some embodiments, the primer is selective for a serotonin (5-HT) receptor. In some embodiments, the primer selectively activates a 5-HT receptor. In some embodiments, the primer is a selective 5-HT receptor agonist, such as a 5-HT₂ or a 5-HT₇ agonist. In some embodiments, the probe is selective for an imidazoline receptor. In some embodiments, the probe selectively activates an imidazoline receptor. In some embodiments, the probe is a selective imidazoline receptor agonist, such as an imidazoline-1 agonist. In some embodiments are disclosed entactogenic compositions capable of selectively activating imidazoline-1 (I₁) receptors together with serotonin-2 (5-HT₂), serotonin-7 (5-HT₇), and/or cannabinoid-1 (CB₁) receptors. By administering a therapeutically effective amount of such compositions, e.g., an I₁ agent together with any of a 5-HT₂, 5-HT₇, and/or CB₁ agent, one may create the entactogenic effects of agents such as MDMA without their negative effects.

Thus, in some aspects, one or more of the entactogenic effects of MDMA and other known entactogens are provided. And, in some aspects, one or more of the adverse effects of MDMA and other known entactogens are diminished or eliminated. Additionally, in some aspects, expanded and enhanced entactogenic effects compared to MDMA and other known entactogens are provided. Among yet other aspects are combinations, compositions, and methods for the treatment of CNS disorders, for mental health conditions and mental disorders, such as psychiatric and neuropsychiatric disorders, and for neurological conditions including neurodevelopmental and neurodegenerative disorders. Among other aspects are combinations, compositions, and methods for the improvement of mental health and mental functioning.

In some disclosed aspects are therapeutic uses of an I₁ agent together with any of a 5-HT₂, 5-HT₇, and/or CB₁ agent. In some aspects are pharmaceutical compositions comprising an I₁ agent together with any of a 5-HT₂, 5-HT₇, and/or CB₁ agent, and a pharmaceutically acceptable carrier, diluent, or excipient. By “an I₁ agent together with any of a 5-HT₂, 5-HT₇, and/or CB₁ agent” and similar terms of combination, it will be appreciated that all possible combinations are meant to be covered, for example any of an I₁ agent together with a CB₁ agent; an I₁ agent together with a 5-HT₂ agent; an I₁ agent together with a 5-HT₇ agent; an I₁ agent together with a CB₁ agent and a 5-HT₂ agent; an I₁ agent together with a CB₁ agent and a 5-HT₇ agent; an I₁ agent together with a 5-HT₂ agent and a 5-HT₇ agent; an I₁ agent together with a 5-HT₂ agent, a CB₁ agent, and a 5-HT₇ agent; and so forth, for any like combinations discussed herein.

### A. General Definitions and Terms

As used in this disclosure and the appended claims, the singular forms “a,” “an,” and “the” include plural referents unless the context clearly dictates otherwise. Thus, for example, reference to “an active agent” includes reference to a combination of one or more active agents, and reference to “an excipient” includes reference to a combination of one or more excipients. While the term “one or more” may be used, its absence (or its replacement by the singular) does not signify the singular only, but simply underscores the possibility of multiple agents or ingredients in particular embodiments.

The terms “comprising,” “including,” “such as,” and “having” are intended to be inclusive and not exclusive (i.e., there may be other elements in addition to the recited elements). Thus, “including” means, and is used interchangeably with, the phrase “including but not limited to.” The term “or” is used herein to mean, and is used interchangeably with, the term “and/or,” unless context clearly indicates otherwise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill, values that are expressed as ranges can assume any specific value or subrange within the stated ranges in different embodiments, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment in which a numerical value is prefaced by “about” or “approximately,” the invention includes an embodiment in which the exact value is recited. For any embodiment of the invention in which a numerical value is not prefaced by “about” or “approximately,” the invention includes an embodiment in which the value is prefaced by “about” or “approximately.” “Approximately” or “about” is intended to encompass numbers that fall within a range of ±10% of a number, in some embodiments within ±5% of a number, in some embodiments within ±1%, in some embodiments within ±0.5% of a number, in some embodiments within ±0.1% of a number unless otherwise stated or otherwise evident from the context (except where such number would impermissibly exceed 100% of a possible value). In some embodiments, the numerical parameters set forth in the written description and attached claims are approximations that can vary depending upon the desired properties sought to be obtained by a particular embodiment (and understood to those in the art).

In some embodiments, the numerical parameters should be construed in light of the number of reported significant digits and by applying ordinary rounding techniques. Notwithstanding that the numerical ranges and parameters setting forth the broad scope of some embodiments are approximations, the numerical values set forth in the specific examples are reported as precisely as practicable. The numerical values presented in some embodiments may contain certain errors necessarily resulting from the standard deviation found in their respective testing measurements.

Throughout this disclosure, various aspects are presented as a range. It should be understood that description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where a stated range includes one or both of the limits, ranges excluding either or both of those included limits are also included in the claimed subject matter. This applies regardless of the breadth of the range.

A list of abbreviations utilized by organic chemists of ordinary skill appears in the first issue of each volume of the Journal of Organic Chemistry; this list is typically presented in a table entitled Standard List of Abbreviations; the list as of the date of this filing is incorporated by reference as if fully set forth herein.

Unless defined otherwise, all technical and scientific terms herein have the meaning as commonly understood by a person having ordinary skill in the art to which this invention belongs, who as a shorthand may be referred to simply as “one of skill.” Further definitions that may assist the reader in understanding the disclosed embodiments are as follows; however, it will be appreciated that such definitions are not intended to limit the scope of the invention, which shall be properly interpreted and understood by reference to the full specification (as well as any plain meaning known to one of skill in the relevant art) in view of the language used in the appended claims. The terminology used herein is for the purpose of describing particular embodiments only, and is not intended to be limiting.

Generally, the nomenclature and terminology used and the procedures performed herein are those known in fields relating to that of one or more disclosed aspects, such as those of biology, pharmacology, neuroscience, organic chemistry, synthetic chemistry, medicinal chemistry, and/or pharmaceutical sciences, and are those that will be well-known and commonly employed in one or more of such fields. Standard techniques and procedures are those generally performed according to conventional methods in the art.

Herein, “in embodiments” may be used as shorthand for and equivalent to “in some embodiments.”

Herein, “entactogenic mindstate” may refer to a state of consciousness characterized by connection with the self. In some embodiments, the entactogenic mindstate promotes or enhances feelings of ecstasy, empathy, openness, compassion, peace, acceptance, healing, open-hearted tenderness, love, oneness, and caring. In some embodiments, the entactogenic mindstate promotes or enhances forgiveness, both of another (the true letting go in one's heart of anger and grudges) and of one's self (the true letting go in one's heart of guilt and shame). In some embodiments, the entactogenic mindstate facilitates one's ability to safely experience and contemplate pain, such as mental pain. In some embodiments, an entactogenic mindstate may refer to the process or feeling of “touching within,” such as described (in a non-drug context) in Brown 2021: “So often, when we feel lost, adrift in our lives, our first instinct is to look out into the distance to find the nearest shore. But that shore, that solid ground, is within us. The anchor we are searching for is connection, and it is internal. To form meaningful connections with others, we must first connect with ourselves, but to do either, we must first establish a common understanding of the language of emotion and human experience.” Purdue University professor of pharmacology and medicinal chemistry David Nichols coined the term “entactogen” in 1986, to provide this meaning—“to touch within” (Holland 2001 at 182 n.2). The term entactogen was adopted subsequent to the original term “empathogen” (meaning “generating a state of empathy”), which was independently suggested in 1983-84 by David Nichols and the psychologist and psychopharmacologist Ralph Metzner (id.). Further elaboration is herein.

Herein, “agent” may refer to a compound that modulates the activity of a target, such as a receptor. In some embodiments, the agent is a ligand for a receptor and modulates the activity of the receptor. In some embodiments, an agent binds to, blocks, activates, inhibits, or otherwise influences (e.g., via an allosteric reaction) activity at a given receptor system. Among the various aspects of the invention are therapeutic uses of an I₁ agent together with any of a 5-HT₂, 5-HT₇, and/or CB₁ agent. In some embodiments, any of an I₁ agent, 5-HT₂ agent, 5-HT₇ agent, and/or CB₁ agent may be a receptor modulator, i.e., modulate activity at an I₁ receptor, 5-HT₂ receptor, 5-HT₇ receptor, and/or CB₁ receptor, respectively.

Herein, “modulate” may refer to an interaction with a target either directly or indirectly so as to alter the activity of the target. In some embodiments, the target is a receptor. Non-limiting examples of altering activity of a target, such as a receptor, are provided herein.

Herein, “modulator” and “receptor modulator” may refer to a receptor ligand, which regulates or modifies the function of a receptor. In some embodiments, the modification is mediated by interaction with a receptor, either directly or indirectly. By way of example only, modulating activity of a receptor may enhance the activity of the receptor, inhibit the activity of the receptor, limit the activity of the receptor, or extend the activity of the receptor. In some embodiments, the alteration in activity is dose dependent. In some embodiments, activation or inhibition of the activity of the receptor may include, but is not limited to, activation of a signal transduction pathway. In some embodiments, any of an I₁ agent, 5-HT₂ agent, 5-HT₇ agent, and/or CB₁ agent may be a receptor modulator, i.e., an I₁ receptor modulator, 5-HT₂ receptor modulator, 5-HT₇ receptor modulator, and/or CB₁ receptor modulator, respectively.

Herein, “activation” may refer to the binding of any of a receptor modulator, agent, or agonist to a receptor, wherein the binding causes a physiological or pharmacological response characteristic of the receptor to be carried out.

Herein, “inhibition” may refer to the binding of any of a receptor modulator, agent, or antagonist to a receptor, wherein the binding totally or partially blocks, dampens, decreases, prevents, or delays a physiological or pharmacological response characteristic of that receptor.

Herein, “agonist” may refer to a compound that elicits or educes an observable positive response from the receptor to which it binds. In some embodiments, an agonist may refer to a chemical species that interacts with and activates a receptor, or that increases or enhances the activity of a receptor, such as one or more of the receptors of the 5-HT₂, 5-HT₇, CB₁, or I₁ family of receptors, and initiates a physiological or pharmacological response characteristic of that receptor. In some embodiments, an agonist may activate a plurality of receptors, non-limiting examples of which include activation at any of 5-HT₂, 5-HT₇, CB₁, I₁, or a combination thereof. In some embodiments, any of an I₁ agent, 5-HT₂ agent, 5-HT₇ agent, and/or CB₁ agent may be an agonist, i.e., an I₁ agonist, 5-HT₂ agonist, 5-HT₇ agonist, and/or CB₁ agonist.

Herein, “partial agonist” may refer to any compound that increases the activity of the receptor to which it binds, but does so at a maximum efficacy beneath 100%. In some embodiments, “partial agonist” may refer to any compound having lower intrinsic activity than an agonist able to reach 100% efficacy. Practically, a “partial agonist” yields a partial or incomplete functional effect in a given functional assay. Because of these qualities, a partial agonist may also serve as a competitive antagonist if competing with agonists possessing the ability to reach 100% efficacy at a given receptor. A partial agonist may produce a lessened biological effect, so that side effects and the likelihood of receptor desensitization may be significantly reduced when utilizing partial agonists in treatment. In some embodiments, a partial agonist may be administered for a longer period of time and may achieve a longer lasting response.

Herein, “inverse agonist” may refer to a compound that produces a conformational change in the receptor to which it binds and renders it “inactive.” Functionally, “inactive,” or “inactivity,” may refer to a state in which signal transduction associated with the receptor is less than that produced at a basal state, i.e., when no agonist is present (“constitutive activity”), or when an antagonist is bound to the receptor. Thus, in the presence of an inverse agonist, the signal transduction pathway associated with the receptor is effectively shut down (Stahl, 2013). That is, while agonists possess “intrinsic efficacy,” inverse agonists possess “negative” intrinsic efficacy.

Non-limiting examples of agonists useful in disclosed embodiments are discussed, inter alia, in the Imidazoline-1(1₁), Cannabinoid-1 (CB₁), Serotonin-2 (5-HT₂), and Serotonin-7 (5-HT₇) sections herein.

Herein, “antagonist” may refer to a compound that binds to a given receptor and partially or fully blocks, dampens, i.e., “inhibits” or “attenuates,” prevents, or delays the biological response characteristic of that receptor. An antagonist may prevent an agonist from binding to, and increasing the activity of, the receptor. Without being bound by theory, an antagonist may accomplish this through binding to any of the active sites which regulate receptor activation directly; an allosteric site or a site other than the active site; or sites not normally involved in the biological regulation of the receptor's activity (Hopkins and Groom, 2002).

In some embodiments, an antagonist can be an “indirect antagonist” or “physiological antagonist,” herein used interchangeably, wherein the antagonist partially or fully blocks, dampens, i.e., “inhibits” or “attenuates,” prevents, or delays the biologic response of a receptor without binding to the receptor.

Herein, “selective” and “selectively binds” may refer to the ability of an agent to bind to a target receptor with greater affinity than it binds to a non-target receptor. In some embodiments, the target receptor is any one of the I₁ receptor, 5-HT₂ receptor, 5-HT₇ receptor, and CB₁ receptor. In some embodiments, any of the agonists and antagonists provided herein may be described as selective.

Although any materials and methods similar or equivalent to those described herein can be used in the embodiments of this disclosure, certain exemplary materials and methods are now described.

### B. Imidazoline 1 (I₁) Agents

In embodiments, a therapeutic combination will comprise an imidazoline-1 agent. Herein, a “therapeutic combination” may refer to two or more active agents administered in combination, so as to achieve a therapeutic effect. “In combination” includes active agents administered as “combination drugs” or as part of “combination therapy,” as the terms are used herein and understood in the art, as well as such other embodiments as will become apparent in view of this disclosure and the general knowledge in the art.

In some embodiments, an “imidazoline-1 agent” (or an “I₁ agent”) may refer to a compound that modulates activity at the imidazoline-1 (I₁) receptor. In embodiments, the I₁ agent binds to, blocks, activates, inhibits, or otherwise influences (e.g., via an allosteric reaction) activity at the I₁ receptor. In embodiments, the I₁ agent acts as a receptor modulator, wherein the I₁ agent alters the function of a receptor to which it binds, the alteration including any of activating the activity of the receptor, inhibiting the activity of the receptor, and activating or inhibiting the activity of the receptor, depending on the dose administered. In some embodiments, the I₁ agent is a “selective agent,” which may refer to an agent that binds to the I₁ receptor system more selectively than receptors not part of the I₁ receptor system. In some embodiments, such selectivity (which may also be termed “specificity” herein), is expressed as a ratio greater than 1.0. In some embodiments, selectivity (as measured via Kᵢ) for the I₁ receptor system is at least 1.1:1, 1.5:1, 2:1, 5:1, 10:1, 15:1, 25:1, 30:1, 50:1, 100:1, etc., as compared to receptors not part of the I₁ receptor system.

In some embodiments, selectivity (as measured via Kᵢ) for the I₁ receptor system is about 1.1:1, as compared to receptors not part of the I₁ receptor system. In some embodiments, selectivity (as measured via Kᵢ) for the I₁ receptor system is about 1.5:1, as compared to receptors not part of the I₁ receptor system. In some embodiments, selectivity (as measured via Kᵢ) for the I₁ receptor system is about 2:1, as compared to receptors not part of the I₁ receptor system. In some embodiments, selectivity (as measured via Kᵢ) for the I₁ receptor system is about 5:1, as compared to receptors not part of the I₁ receptor system. In some embodiments, selectivity (as measured via Kᵢ) for the I₁ receptor system is at least about 10:1, as compared to receptors not part of the I₁ receptor system. For example, in some embodiments, selectivity (as measured via Kᵢ) for the I₁ receptor system is about 10:1, as compared to receptors not part of the I₁ receptor system. In some preferred embodiments, selectivity (as measured via Kᵢ) for the I₁ receptor system is about 25:1, as compared to receptors not part of the I₁ receptor system. In other preferred embodiments, selectivity (as measured via Kᵢ) for the I₁ receptor system is about 30:1, as compared to receptors not part of the I₁ receptor system. In other preferred embodiments, selectivity (as measured via Kᵢ) for the I₁ receptor system is at least about 30:1, as compared to receptors not part of the I₁ receptor system. In some embodiments, selectivity (as measured via Kᵢ) for the I₁ receptor system is about 50:1, as compared to receptors not part of the I₁ receptor system. In some embodiments, selectivity (as measured via Kᵢ) for the I₁ receptor system is at least about 100:1, as compared to receptors not part of the I₁ receptor system.

In some embodiments, selectivity (as measured via Kᵢ) for the I₁ receptor system is about 1.1:1, as compared to a receptor of the alpha-2 (α2) adrenergic receptor system. In some embodiments, selectivity (as measured via Kᵢ) for the I₁ receptor system is about 1.5:1, as compared to an α2 adrenergic receptor. In some embodiments, selectivity (as measured via Kᵢ) for the I₁ receptor system is about 2:1, as compared to an α2 adrenergic receptor. In some embodiments, selectivity (as measured via Kᵢ) for the I₁ receptor system is about 5:1, as compared to an α2 adrenergic receptor. In some embodiments, selectivity (as measured via Kᵢ) for the I₁ receptor system is at least about 10:1, as compared to an α2 adrenergic receptor. For example, in some embodiments, selectivity (as measured via Kᵢ) for the I₁ receptor system is about 10:1, as compared to an α2 adrenergic receptor. In some preferred embodiments, selectivity (as measured via Kᵢ) for the I₁ receptor system is about 25:1, as compared to an α2 adrenergic receptor. In other preferred embodiments, selectivity (as measured via Kᵢ) for the I₁ receptor system is about 30:1, as compared to an α2 adrenergic receptor. In other preferred embodiments, selectivity (as measured via Kᵢ) for the I₁ receptor system is at least about 30:1, as compared to an α2 adrenergic receptor. In some embodiments, selectivity (as measured via Kᵢ) for the I₁ receptor system is about 50:1, as compared to an α2 adrenergic receptor. In some embodiments, selectivity (as measured via Kᵢ) for the I₁ receptor system is at least about 100:1, as compared to an α2 adrenergic receptor.

In some embodiments, an I₁ agent is an imidazoline-1 receptor agonist. Broadly, an “imidazoline-1 receptor agonist” (or an “imidazoline-1 agonist,” “I₁ receptor agonist,” or “I₁ agonist”) is a compound that initiates a biological response when bound to an I₁ receptor. In some embodiments, the I₁ agonist has a high specificity for I₁ receptors. In some embodiments, the I₁ agonist has a low specificity for receptors not part of the imidazoline-1 receptor system. Herein, in embodiments, and where the terms are not otherwise defined or intended to have another meaning necessary to provide technical effect, modifying terms such as “high” and “low” are used comparatively, meaning a “high” specificity is merely higher than a “low” specificity.

In embodiments, the I₁ agonist binds to both alpha-2 adrenergic (α₂ adrenergic) and imidazoline-1 receptors, but possesses a lower specificity for α₂ adrenergic receptors than imidazoline-1 receptors, so that the agent's selectivity for imidazoline-1 is greater than its selectivity for α₂ adrenergic receptors.

In some embodiments, an I₁ agent is an imidazoline-1 receptor antagonist. Broadly, an “imidazoline-1 receptor antagonist” (or an “imidazoline-1 antagonist,” “I₁ receptor antagonist,” or “I₁ antagonist”) is a compound that inhibits a biological response when bound to an I₁ receptor.

In embodiments, the I₁ antagonist has a low specificity for receptors not part of the imidazoline-1 receptor system. In embodiments, the I₁ antagonist binds to both alpha-2 (α₂) adrenergic and imidazoline-1 receptors, but possesses a lower specificity for α₂ adrenergic receptors than imidazoline-1 receptors, so that the agent's selectivity for imidazoline-1 is greater than its selectivity for α₂ adrenergic receptors.

While in some embodiments, a selective agent has 100-fold or greater selectivity for its target receptors, relative to all other receptors, in many embodiments, a selective agent will have less than 100-fold selectivity. Accordingly, while in some embodiments an I₁ selective agent has 100-fold or greater selectivity for I₁ receptors, relative to all other receptors, in other embodiments, an I₁ selective agent will have less than 100-fold selectivity. For example, in some such embodiments, an I₁ selective agent has about 30-fold or greater selectivity for I₁ receptors, relative to all other receptors. In some embodiments an I₁ selective agent has any degree of selectivity for I₁ receptors, relative to all other receptors, wherein the degree of selectivity is as further described herein. In some embodiments an I₁ selective agent has about 30-fold or greater selectivity for I₁ receptors, relative to α₂ adrenergic receptors. In some embodiments an I₁ selective agent is moxonidine or rilmenidine. In some embodiments, a selective agent will have selectivity for its target receptors, relative to one or more other types of receptors, but not all other receptors.

In some embodiments, the imidazoline agents will have greater than about 30-fold selectivity for I₁ relative to α₂ receptors. For example, the imidazoline agents moxonidine and rilmenidine have been shown to be about 33- and 30-fold selective respectively, for I₁ relative to α₂ receptors (Ziegler, 1996):

| | I₁ Imidazoline Sites | α₂ Adrenergic Sites | Selectivity Ratio |
|---|---|---|---|
| Moxonidine | 2.3 ± 0.5 | 75 ± 8 | 32.61 |
| Rilmenidine | 6.1 ± 1.5 | 180 ± 14 | 29.51 |
| Clonidine | 1.0 ± 0.3 | 3.8 ± 1.0 | 3.80 |
| Lofexidine | 1.9 ± 0.7 | 6.9 ± 0.7 | 3.63 |

In some embodiments, the I₁ agent is an inverse agonist. In some embodiments, the I₁ inverse agonist has a high specificity for I₁ receptors. In some embodiments, the I₁ inverse agonist has a low specificity for receptors not part of the imidazoline-1 receptor system. In some embodiments, the I₁ inverse agonist binds to both alpha-2 adrenergic (α₂ adrenergic) and imidazoline-1 receptors, but possesses a lower specificity for α₂ adrenergic receptors than imidazoline-1 receptors, so that the agent's selectivity for imidazoline-1 is greater than its selectivity for α₂ adrenergic receptors.

Presently, additional I₁ agonists with no detectable affinity or activity for the α₂-adrenergic receptor are known and in some embodiments herein may be used, non-limiting examples of which include LNP599 [3-chloro-2-methyl-phenyl)-(4-methyl-4,5-dihydro-3H-pyrrol-2-yl)-amine], which, in addition to its inactivity at α₂, also possesses a nanomolar affinity for I₁; LNP509 [dicyclopropylmethyl-(4,5-dimethyl-4,5-dihydro-3H-pyrrol-2-yl)-amine], LNP630 [(2,6-dichloro-phenyl)-(4-methyl-4,5-dihydro-1H-imidazol-2-yl)-amine], LNP911 [2-(2-chloro-4-iodo-phenylamino)-5-methyl-pyrroline], and LNP906 [2-(5-azido-2-chloro-4-iodo-phenyl-amino)-5-methyl-pyrroline], the latter of which has been shown to antagonize the I₁ receptor system, and possess photoaffinity properties (Urosevic et al., 2004).

In some embodiments, clonidine is used in a therapeutic combination or pharmaceutical composition and, when administered in a therapeutically effective amount, and according to the invention, is capable of eliciting an entactogenic mindstate. In some embodiments, clonidine or other I₁ agonists exhibiting affinity for α₂-adrenergic receptors will be used. In some embodiments, the I₁ agent is clonidine. In other embodiments, the I₁ agent is rilmenidine or moxonidine, or another selective I₁ agonist.

In embodiments, I₁ agents are used in therapeutic combinations with one or more of a 5-HT₂, 5-HT₇, and/or CB₁ agent. In such combinations, the entactogenic effects of the I₁ receptor system are elicited, while foregoing the undesirable adverse effects associated with MDMA and other known entactogens.

### a. Exemplary I₁ Agents Useful in Embodiments of the Invention

In some embodiments, non-limiting examples of I₁ agents useful in a therapeutic combination include: 1-(4-Aminobutyl)guanidine (agmatine), 2-(1-phenylpropan-2-yl)-4,5-dihydro-1H-imidazole (BDBM50091347), N-(2,6-Dichlorophenyl)-4,5-1H-imidazol-2-amine (clonidine), N-(diaminomethylidene)-2-(2,6-dichlorophenyl)acetamide (guanfacine), 1-(3-chlorophenyl)piperazine (mCPP), (RS)-1-(1,3-Benzo-dioxol-5-yl)-N-methylpropan-2-amine (MDMA), 3,5-dimethyladamantan-1-amine (memantine), 4-Chloro-N-(4,5-dihydro-1H-imidazol-2-yl)-6-methoxy-2-methylpyrimidin-5-amine (moxonidine), 2-(naphthalen-1-ylmethyl)-4,5-dihydro-1H-imidazole (naphazoline), 3-(4,5-Dihydro-1H-imidazol-2-ylmethyl)-2,4-dimethyl-6-tert-butyl-phenol (oxymetazoline), N-(dicyclopropylmethyl)-4,5-dihydro-1,3-oxazol-2-amine (rilmenidine), (RS)-2-(1,2,3,4-tetrahydronaphthalen-1-yl)-4,5-dihydro-1H-imidazole (tetryzoline, also known as tetrahydrozoline), 5-Chloro-N-(4,5-dihydro-1H-imidazol-2-yl)benzo[c][1,2,5]thiadiazol-4-amine (tizanidine), N-(2-Chloro-4-methylphenyl)-4,5-dihydro-1H-imidazol-2-amine (tolonidine), and additional compounds below; and any salts, isomers (including structural isomers and stereoisomers, such as enantiomers), precursors, prodrugs, metabolites, derivatives, analogs, isotopologs, or variants thereof, or a combination thereof (see, e.g., Nikolic et al., 2012). Certain other exemplary I₁ agents, useful in disclosed embodiments, include idazoxan, p-aminoclonidine, benazoline, BDF 6143, and p-iodoclonidine.

One of skill will appreciate the ability to determine which other compounds are useful agents in the practice of disclosed embodiments, by reference to the teachings herein in combination with the general knowledge in the art, such as by reference to DrugCentral, DrugBank, and other compendia of drug data.

### C. Cannabinoid-1 (CB₁) Agents

In some embodiments, a therapeutic combination will comprise a cannabinoid-1 agent. In some embodiments, a “cannabinoid-1 agent” (or a “CB₁ agent”) may refer to a compound that modulates a cannabinoid-1 (CB₁) receptor. In some embodiments, the CB₁ agent binds to, blocks, activates, inhibits, or otherwise influences (e.g., via an allosteric reaction) activity at the CB₁ receptor. In some embodiments, the CB₁ agent acts as a receptor modulator, wherein the CB₁ agent alters the function of a receptor to which it binds, the alteration including any of activating the activity of the receptor, inhibiting the activity of the receptor, and activating or inhibiting the activity of the receptor, depending on the dose administered.

In some embodiments, the CB₁ agent is a “selective agent,” which may refer to an agent that binds to the CB₁ receptor system more selectively than receptors not part of the CB₁ receptor system. In some embodiments, such selectivity (also termed “specificity” herein), is expressed as a ratio greater than 1.0. In some embodiments, selectivity (as measured via Kᵢ) for the CB₁ receptor system is at least 1.1:1, 1.5:1, 2:1, 5:1, 10:1, 15:1, 25:1, 50:1, 100:1, etc., as compared to receptors not part of the CB₁ receptor system.

It will be readily appreciated that here (as elsewhere herein, for other receptor systems), selectivity for a single receptor system (such as the CB₁ receptor system) need not require selectivity as compared to all other receptor systems, or that a compound be tested at every possible receptor system to determine whether such compound is indeed “selective,” but rather that the term may be used and given the meaning as understood by those of skill (e.g., Ray 2010), and in view of the other receptor systems disclosed herein.

In some embodiments, a CB₁ agent is a CB₁ receptor agonist. Broadly, a “CB₁ receptor agonist” (or a “cannabinoid-1 agonist,” “CB₁ receptor agonist,” or “CB₁ agonist”) is a compound that initiates a biological response when bound to a CB₁ receptor. In some embodiments, the CB₁ agonist has a high specificity for CB₁ receptors. In embodiments, the CB₁ agonist has a low specificity for receptors not part of the endocannabinoid system.

In some embodiments, a CB₁ agent is a CB₁ receptor antagonist. Broadly, a “CB₁ receptor antagonist” (or a “cannabinoid-1 antagonist,” “CB₁ receptor antagonist,” or “CB₁ antagonist”) is a compound that inhibits a biological response when bound to a CB₁ receptor. In some embodiments, the CB₁ antagonist has a high specificity for CB₁ receptors. In some embodiments, the CB₁ antagonist has a low specificity for receptors not part of the endocannabinoid system.

In some embodiments, the CB₁ agent is an inverse agonist. In some embodiments, the CB₁ inverse agonist has a high specificity for CB₁ receptors. In some embodiments, the CB₁ inverse agonist has a low specificity for receptors not part of the endocannabinoid system.

Functionally, the CB₁ receptor binds the main psychoactive ingredient of Cannabis (i.e., marijuana), (−)-trans-Δ⁹-tetrahydrocannabinol (Δ⁹-THC), and mediates most of the CNS effects of Δ⁹-THC (Zimmer et al., 1999). Δ⁹-THC is the most widely-known cannabinoid derived from cannabis, particularly because of its intoxicating effects, resulting in the “high” associated with cannabis use.

THC and other cannabinoids may be ingested via inhalation of combusted Cannabis flower, such as marijuana packed in a pipe (a bowl), in a marijuana cigarette (a joint), in a hollowed-out cigar filled with marijuana (a blunt), in a bong, including those using water; as a vaporized extract, isolate, distillate, or concentrate (e.g., as wax, shatter, budder, crumble, live resin, various oils, and others, including as dabs), or from a pen or vape pen containing cartridges; or ingested via foods (edibles) in which Cannabis or an extract thereof is infused or incorporated, including gummies, chocolates, pills, sweets, brownies, cookies, cakes, candies, and the like. As each administration method inherently has different absorption mechanisms, duration of effects and elapsed time before effects are felt may vary, but knowledge of such is within ordinary skill. For example, administration via inhalation is felt by a subject almost immediately, while ingestion via an edible may take 30 minutes to an hour or more to take effect (depending on age, weight, height, metabolism, etc.). Diverse administration methods of Cannabis and cannabinoids exist, too numerous to list exhaustively here, but will be readily appreciated by those in the art.

Besides the principal THC isomer Δ⁹-THC, numerous isomers exist, such as Δ⁸-THC. Other naturally-occurring, synthetic (including bioengineered) isomers, metabolites (e.g., THC-COOH (11-nor-9-carboxy-THC)), salts, derivatives, analogs, or variants of THC will be recognized or known to those of skill, or be able to be created by the practice of ordinary skill. In embodiments, “THC” thus may refer to, or be an equivalent of (at least in certain embodiments), such naturally-occurring or synthetic isomers, metabolites, salts, derivatives, analogs, and variants, including Δ³-THC, Δ⁸-THC, Δ⁹-THC, Δ¹⁰-THC, THCV, THCO, THCP, and THCB. In embodiments, “THC” may be shorthand, to refer to Δ⁹-THC and its isomers, unless context indicates otherwise. In embodiments, where context allows, “THC” may refer to a single compound, e.g., any of Δ³-THC, Δ⁸-THC, Δ⁹-THC, Δ¹⁰-THC, THCV, THCO, THCP, and THCB.

In embodiments, the CB₁ agent is a cannabinoid. “Cannabinoid” may refer to one of a class of compounds that are structurally or functionally related. Cannabinoids are functionally related in that they each act on one or more of the CB₁, CB₂, and GPR₅₅ receptors. Generally, ligands for these receptors include what may be termed “endocannabinoids” (i.e., cannabinoids produced endogenously in the body by humans and other mammals), “phytocannabinoids” (i.e., cannabinoids from Cannabis or other plants), and “synthetic cannabinoids” (i.e., synthetic analogs of natural cannabinoids).

In some embodiments, the CB₁ agent is an endocannabinoid. In some embodiments, the endocannabinoids is any of anandamide, oleamide, 2-arachidonoylglycerol (2-AG), N-arachidonoyl ethanolamide (AEA), N-arachidonoylglycine (NAGly), N-arachidonoyldopamine (NADA), 2-arachidonoyl-glyceryl ether (2-AGE), and 9-octadecenoamide (oleamide). In embodiments, endocannabinoids are the same as those endogenously produced, but are administered exogenously.

In some embodiments, the CB₁ agent is a phytocannabinoid. In some embodiments, the CB₁ agent is a natural phytocannabinoid, wherein “natural phytocannabinoid” may refer to a cannabinoid that occurs in nature, and is produced by a plant (e.g., a Cannabis plant). In some embodiments, the phytocannabinoid is any of tetrahydrocannabinol (THC), cannabidiol (CBD), cannabichromene (CBC), cannabidinodiol (also known as cannabinodiol) (CBND, CBDL), cannabielsoin (CBE), cannabicyclol (CBL), cannabicitran (CBTC), cannabitriol (CBT), cannabivarin (CBV), cannabigerol monomethyl ether (CBGM), cannabidiphorol (CBDP), tetrahydrocannabiphorol (THCP), and iso-tetrahydrocannabinol (iso-THC).

In embodiments, the CB₁ agent will be a phytocannabinoid from a Cannabis plant. Broadly, “cannabis” may refer to any of the plants belonging to the family Cannabaceae, including Cannabis sativa, Cannabis indica, and Cannabis ruderalis plants, cultivars and chemovars thereof, and any plant parts or tissue, such as preferably the female inflorescences and top leaves, but including stalks, stems, roots, etc., and preparations such as extracts and ingestibles or consumables made therefrom.

In embodiments, a phytocannabinoid (including from Cannabis) is any of CBD, CBDV, CBND, CBDL, CBE, CBL, CBTC, CBT, CBV, CBG, CBGV, CBGM, CBDP, THC (and further including, e.g., THCP and THCV), and iso-THC, as well as their acidic forms, their propyl, methyl, and ethyl homologues, and the acid forms of those homologues (i.e., their acidic propyl, acidic methyl, and acidic ethyl homologue forms).

In embodiments, a cannabinoid will be any one or more of those as set forth and described by Radwan et al. in Cannabinoids, Phenolics, Terpenes and Alkaloids of Cannabis, Molecules, 26(9), 2774 (2021), which is incorporated by reference as if fully set forth herein. In general, but without being bound by theory, cannabinoids according to such categorization include compounds with a characteristic C21 terpenophenolic backbone that are part of one of 11 cannabinoid sub-classes, namely: cannabichromene (CBC)-type, cannabidiol (CBD) type, cannabielsoin (CBE) type, cannabigerol (CBG) type, cannabicyclol (CBL) type, cannabinol (CBN) type, cannabinodiol (CBND) type, cannabitriol (CBT) type, (−)-Δ⁸-trans-tetrahydrocannabinol (Δ⁸-THC) type, (−)-Δ⁹-trans-tetrahydrocannabinol (Δ⁹-THC) type, and miscellaneous-type cannabinoids. Non-limiting examples of such cannabinoids, all of which will be understood to be useful in the practice of the invention, are known by reference to the disclosure of Radwan 2021 and the below. For example, in embodiments, the CB₁ agent is one or more “Δ⁹-THC-type cannabinoids” which include Δ⁹-THC-C₅, Δ⁹-THCAA-C₅, Δ⁹-THCAB-C₅, Δ⁹-THC-C₄, Δ⁹-THCAA-C₄, Δ⁹-THCV, Δ⁹-THCVAA, Δ⁹-THCO, Δ⁹-THCOAA, Δ⁹-THC-aldehyde, β-fenchyl (−)-Δ9-trans-tetrahydro-cannabinolate, α-fenchyl (−)-Δ9-trans-tetrahydrocannabinolate, epi-bornyl (−)-Δ9-trans-tetrahydro-cannabinolate, bornyl (−)-Δ9-trans-tetrahydrocannabinolate, α-terpenyl (−)-Δ9-trans-tetrahydro-cannabinolate, 4-terpenyl (−)-Δ9-trans-tetrahydrocannabinolate, α-cadinyl (−)-Δ9-trans-tetrahydro-cannabinolate, γ-eudesmyl (−)-Δ9-trans-tetrahydrocannabinolate, 8α-hydroxy-(−)-Δ9-trans-tetrahydro-cannabinol, 8β-hydroxy-(−)-Δ9-trans-tetrahydrocannabinol, 11-acetoxy-(−)-Δ9-trans-tetrahydro-cannabinolic acid A, 8-oxo-(−)-Δ9-trans-tetrahydrocannabinol, cannabisol, (−)-Δ9-trans-tetrahydro-cannabiphorol, and (−)-Δ9-trans-tetrahydrocannabihexol.

In embodiments, the CB₁ agent is one or more “Δ⁸-THC-type cannabinoids” which include Δ⁸-THC, Δ⁸-THCA, 10α-OH-Δ⁸-THC, 10β-OH-Δ⁸-THC, and 10a-α-hydroxy-10-oxo-Δ8-THC.

In embodiments, the CB₁ agent is one or more “CBG-type cannabinoids” which include (E)CBG, (E)CBGA, (E)CBGG, (E)CBGAM, (E)CBGV, (E)CBGVA, (Z)CBGA, 5-acetyl-4-hydroxy-cannabigerol, (±)-6,7-trans-epoxycannabigerolic acid, (±)-6,7-cis-epoxy-cannabigerolic acid, (±)-6,7-cis-epoxycannabigerol, (±)-6,7-trans-epxoycannabigerol, camagerol, and sesquicannabigerol.

In embodiments, the CB₁ agent is one or more “CBD, CBND, CBE, and CBL-type cannabinoids” which include CBD-C₅, CBDA-C₅, CBDM-C₅, CBD-C₄, CBDV, CBDVA, CBD-C₁, CBDH, CBDP, CBDD, CBND-C₃, CBND-C₅, CBE-C₅, CBEAA-C₅, CBEAB-C₅, CBE-C₃, CBEAB-C₃, CBL, CBLA, and CBLV.

In embodiments, the CB₁ agent is one or more “CBC and CBN-type cannabinoids” which include CBC, CBCA, ±CBCV, +CBCV, CBCVA, 4-acetoxy-CBC, (±)-3″-hydroxy-Δ4″-cannabichromene, (−)-7-hydroxy-cannabichromane, CBC-C₃, CBN-C₅, CBNA-C₅, CBN-C₄, CBN-C₃, CBN-C₂, CBN-C₁, CBNM-C₅, 8-OH-CBN, 8-OH-CBNA, 1'S—OH-CBN, and 4-terpenyl-cannabinolate.

In embodiments, the CB₁ agent is one or more “CBT-type cannabinoids” which include (−)-trans-CBT-C₅, (+)-trans-CBT-C₅, (±)-cis-CBT-C₅, (±)-trans-CBT-C₃, CBT-C₃-homologue, (−)-trans-CBT-OEt-C₅, (−)-trans-CBT-OEt-C₃, 8,9-Di-OH-CBT-C₅, and CBDA-C₅, and 9-OH-CBT-C₅ ester.

In embodiments, the CB₁ agent is one or more “miscellaneous-type cannabinoids” which include DCBF-C₅, CBF-C₅, OH-iso-HHCV-C₃ OTHC, cannabicitran, cis-Δ⁹-THC, CBCON-C₅, CBR, CBTT, CBCN-C₅, CBCN—C₃, cis-iso-Δ⁷-THCV, trans-iso-Δ⁷-THCV, trans-iso-Δ⁷-THC, CBCNB, CBCNC, CBCND, (−)-(7R)-cannabicoumarononic acid, 4-acetoxy-2-geranyl-5-hydroxy-3-n-pentylphenol, 2-geranyl-5-hydroxy-3-n-pentyl-1,4-benzoquinone, 5-acetoxy-6-geranyl-3-n-pentyl-1,4-benzoquinone, CBM, CBX, 10α-hydroxy-Δ^{9,11}-hexahydrocannabinol, 9β,10β-epoxyhexahydrocannabinol, 9α-hydroxyhexahydrocannabinol, 7-oxo-9α-hydroxyhexahydrocannabinol, 10α-hydroxyhexahydrocannabinol, 10aR-hydroxyhexahydro-cannabinol, and 9α-hydroxy-10-oxo-Δ^{6a,10a}-THC.

In embodiments, the CB₁ agent, the cannabinoid, the phytocannabinoid, or the phytocannabinoid from Cannabis, is a hydrogenated derivative. For example, a hydrogenated derivative of THC is hexahydrocannabinol (HHC), which is a naturally occurring phytocannabinoid that has been identified as a trace component in certain Cannabis, and which can also be and has been produced synthetically by hydrogenation of Cannabis extracts, and has also been synthesized as both the (+)-HHC and (−)-HHC isomers from citronellal and olivetol. As with other cannabinoids discussed herein, HHC may be administered by a suitable vaporization device such as a disposable vape, or by any other suitable administration means described herein. Other structurally related hydrogenated derivatives, also within the scope of the disclosure and useful as CB₁ agents, cannabinoids, phytocannabinoids, or phytocannabinoids from Cannabis, include cannabiripsol, 9α-hydroxyhexahydrocannabinol, 7-oxo-9α-hydroxyhexahydro-cannabinol, 10α-hydroxyhexahydrocannabinol, 10aR-hydroxyhexahydro-cannabinol and 1′S-hydroxy-cannabinol, 10α-hydroxy-Δ(9,11)-hexahydrocannabinol and 9β,10β-epoxyhexa-hydrocannabinol. Other related compounds, which are sometimes also referred to as “HHC”s, and which include as examples 9-Nor-9β-hydroxyhexahydrocannabinol (9-Nor-9Beta-HHC), 9-Hydroxyhexahydro-cannabinol (9-OH-HHC), 11-Hydroxyhexahydrocannabinol (11-OH-HHC and 7-OH-HHC), also may be utilized.

In some embodiments, the CB₁ agent is cannabidiol (CBD). CBD, unlike THC, does not produce intoxicating effects in humans, and in fact can antagonize such effects by THC. CBD is an inverse agonist of CB₂ receptors, and acts as an indirect antagonist of CB₁ and CB₂ cannabinoid receptor agonists (i.e., as an antagonist to other ligands at CB₁ and CB₂ receptors). While THC directly activates CB₁ and CB₂ receptors, CBD does not bind to either, but instead impacts them indirectly. The pharmacology of CBD is thus more enigmatic, involving interactions with multiple neurochemical systems, including serotonergic and adenosinergic systems. Mechanisms that may be related to the actions of CBD include activation of TRPV1 channels, inhibition of uptake and metabolism of the endocannabinoid anandamide (in part through FAAH inhibition), inhibition of adenosine uptake, antagonism of GPR₅₅ and agonism of PPARγ receptors, and increase of intracellular calcium. CBD may act as an agonist of serotonergic 5-HT_{1A} receptors.

Accordingly, it will be appreciated that, in some embodiments, an “agent” need not be an “agonist” (e.g., a CB₁ agent need not be a CB₁ agonist, and may instead be an antagonist, inverse agonist, receptor modulator, etc.), and neither an “agent” nor an “agonist” need be limited to activity at a specific receptor or receptor system, but may have activity across multiple such systems, having multiple mechanisms of action (however, in some embodiments, including certain preferred embodiments herein, an agent or an agonist at one receptor or receptor system will be selective therefor, with minimal or no activity at other sites).

It will be appreciated that in fresh Cannabis plant material, cannabinoids are almost exclusively found in their “acidic” form (e.g., THCA, CBDA, CBCA), rather than their “neutral” form (e.g., THC, CBD, CBC) (and further, may have isomers, e.g., THCA-A and THCA-B). In embodiments, a CB₁ agent will be a decarboxylated cannabinoid, including, e.g., a fully decarboxylated, a substantially decarboxylated, a partially decarboxylated, or relatively decarboxylated cannabinoid. In embodiments, a CB₁ agent will be an acidic cannabinoid (a non-decarboxylated cannabinoid).

In some embodiments, a CB₁ agent is a naturally-occurring phytocannabinoid from a Cannabis plant, and is any one or more of CBD, CBDV, CBND, CBDL, CBE, CBL, CBTC, CBT, CBV, CBG, CBGV, CBGM, CBDP, THC (and further including, e.g., THCP and THCV), and iso-THC.

Cannabinoids also may appear in their propyl homologue form, e.g., as cannabichromevarin (CBCV), cannabigerovarin (CBGV), and the like. Cannabinoids also may be found in the acidic versions of these homologue forms (e.g., in acidic propyl, acidic methyl, and acidic ethyl homologue forms). In some embodiments, the CB₁ agent is an acidic propyl, acidic methyl, or acidic ethyl homologue.

In embodiments, the CB₁ agent is a synthetic cannabinoid. In embodiments, a synthetic cannabinoid is a synthetic classical cannabinoid, a synthetic non-classical cannabimimetic compound, a hybrid cannabinoid, an aminoalkylindoles, and/or an eicosanoids, such as AKB48, CP55940, CP-47497, CP-47497 C8, JWH-015, WIN55212-2, JWH-018, AM-2201, JWH-122, JWH-073, JWH-081, JWH-200, JWH-210, UR-144, XLR11. In embodiments, a synthetic cannabinoid is a cannabinoid glycoside (“cannaboside”) or acetylated cannabinoid, e.g., as in U.S. patent application Ser. Nos. 16/110,728 and 16/110,954.

Synthetic cannabimimetic compounds in some embodiments include those that generally act as CB₁ receptor agonists, as described herein, and may include those from numerous structural classes, such as adamantoylindoles or indazole carboxamides (e.g., 5F-AKB-48, APICA, STS-135); benzimidazoles (e.g., AZ-11713908, AZD-1940); phenylacetylindoles (e.g., JWH-250, RCS-8); cyclohexylphenols (e.g., CP-47,947, CP-55,940); dibenzopyrans (e.g., JWH-051, JWH-056); eicosanoids (e.g., AM-883, AM-1346, O-585, O-689); naphtylindenes (e.g., JWH-171, JWH-176); indazole carboxamides (e.g., AB-PINACA, AB-FUBINACA); indazole-3-carboxamides (e.g., AB-CHMINACA, AB-FUBINACA, PX-2, PX-3); indole-3-carboxamides (e.g., CUMYL-BICA, CUMYL-CBMICA, Org 28312, Org 28611); indole-3-carboxylates or aryloxycarbonylindoles (e.g., FDU-PB-22, FUB-PB-22); naphthoylindazoles (e.g., THJ-018, THJ-2201); naphthoylindoles (e.g., JWH-007, JWH-018); naphthoylindoles (e.g., JWH-398, AM-1221); naphthoylindole (e.g., AM-2201, WIN-55,212-2); naphthoylindoles (e.g., JWH-073, JWH-200); phenylacetylindoles (e.g., JWH-167, JWH-203); pyrazolecarboxamides (e.g., 5F-AB-FUPPYCA, AB-CHFUPYCA); pyrrolobenzoxazines or naphtoylindoles (e.g., WIN 55,212-2); quinolinyl esters or aryloxycarbonylindoles (e.g., PB-22, 5F-PB-22); tetramethylcyclopropylcarbonylindoles (e.g., A-796,260, A-834,735, XLR-11, XLR-12); tetramethylcyclopropylcarbonylindazoles (e.g., FAB-144); and tetramethylcyclopropylcarbonylindoles (e.g., UR-144, XLR-11).

In embodiments, the CB₁ agent will be a cannabinoid that is structurally related in that is has a structure similar to any one or more of the compounds above, is a direct modification of any such compound, or is otherwise a salt, isomer (including a structural isomer or stereoisomer, such as an enantiomer), precursor, prodrug, metabolite, derivative, analog, isotopolog, or variant thereof, including combinations thereof. Compounds have been described in, e.g., U.S. Pat. Nos. 5,532,237; 5,605,906; 5,631,297; 6,410,588; 6,610,737; 6,630,507 at cols. 3:36-6:66; U.S. Pat. No. 6,274,635 at cols. 8:63-20:11; and U.S. Pat. No. 8,071,641 at cols. 5:38-6:26; and references cited; Silva et al., 2020, and numerous other cannabinoids will be recognized or known to those of skill, or be able to be created through the practice of ordinary skill.

In embodiments, a therapeutic combination comprises an I₁ agent in combination with a CB₁ agent, such as any disclosed CB₁ agent, and optionally a 5-HT₂ agent and/or 5-HT₇ agent. In some embodiments, a therapeutic combination comprises an I₁ agent in combination with an exemplary CB₁ agent, such as listed below. In embodiments, administration of the therapeutic combination including a CB₁ agent provides a therapeutic effect, for example producing one or more entactogenic effects or an entactogenic mindstate.

### a. Exemplary CB₁ Agents Useful in Embodiments of the Invention

In some embodiments, the CB₁ agent is any of 2-{[(5Z,8Z,11Z,14Z)-Icosa-5,8,11,14-tetraen-1-yl]oxy}propane-1,3-diol (2-AGE), {1-[(Tetrahydro-2H-pyran-4-yl)methyl]-1H-indol-3-yl}-(2,2,3,3-tetramethylcyclopropyl)methanone (A-834,735), arachidonyl-2′-chloroethylamide (ACEA)N-(1-(cyclohexyl-methyl)-2-((5-ethoxypyridin-2-yl)methyl)-1H-benzo[d]imidazol-5-yl)-N-methylthiophene-2-sulfonamide (AZ-11713908), N-{1-[(4,4-difluorocyclohexyl)methyl]-2-(1,1-dimethylethyl)-1H-benzimidazol-5-yl}ethanesulfonamide (AZD-1940), 6,6,9-Trimethyl-3-pentyl-benzo[c]chromen-1-ol (cannabinol; CBN), 5-(1,1-dimethylheptyl)-2-[(1R,3S)-3-hydroxycyclohexyl]-phenol (CP-47497), 2-[(1R,2R,5R)-5-Hydroxy-2-(3-hydroxypropyl)cyclohexyl]-5-(2-methyloctan-2-yl)phenol (CP-55940), (6aR, 10aR)-9-(hydroxymethyl)-6,6-dimethyl-3-(2-methyloctan-2-yl)-6H,6aH, 7H, 10H,10aH-benzo[c]isochromen-1-ol (HU-210), 1-Pentyl-2-methyl-3-(1-naphthoyl)indole (JWH-007), ((6aR,10aR)-6,6-Dimethyl-3-(2-methyloctan-2-yl)-6a,7,10,10a-tetrahydrobenzo[c]chromen-9-yl)methanol (JWH-051), (1-heptyl-5-phenyl-1H-pyrrol-3-yl)-1-naphthalenyl-methanone (JWH-146), 1-([(1E)-3-Pentylinden-1-ylidine]methyl)naphthalene (JWH-176), (1-(2-Morpholin-4-ylethyl)indol-3-yl)-naphthalen-1-ylmethanone (JWH-200), 1-Pentyl-3-(4-chloro-1-naphthoyl)indole (JWH-398), [1-(cyclohexylmethyl)-7-methoxyindol-3-yl]-[(3S)-3,4-dimethylpiperazin-1-yl]methanone (Org 28611), 5-(4-Chlorophenyl)-1-(2,4-dichlorophenyl)-4-methyl-N-(piperidin-1-yl)-1H-pyrazole-3-carboxamide (rimonabant), tetrahydrocannabinol (THC), (11R)-2-Methyl-11-[(morpholin-4-yl)methyl]-3-(naphthalene-1-carbonyl)-9-oxa-1-azatricyclo[6.3.1.04,12]dodeca-2,4(12), 5,7-tetraene (WIN55212-2), (1-(5-fluoropentyl)-1H-indol-3-yl)(2,2,3,3-tetramethylcyclopropyl)methanone (XLR11), and any salts, isomers (including structural isomers and stereoisomers, such as enantiomers), precursors, prodrugs, metabolites, derivatives, analogs, isotopologs, or variants thereof, or a combination thereof.

One of skill will appreciate the ability to determine which other compounds are useful agents in the practice of disclosed embodiments, by reference to the teachings herein in combination with the general knowledge in the art, such as by reference to DrugCentral, DrugBank, and other compendia of drug data.

### D. Serotonin-2 (5-HT₂) Agents

In some embodiments, a therapeutic combination will comprise a serotonin-2 agent. In some embodiments, a “serotonin-2 agent” may refer to a compound that modulates the activity of a serotonin-2 (5-HT₂) receptor. In some embodiments, the 5-HT₂ agent binds to, blocks, activates, inhibits, or otherwise influences (e.g., via an allosteric reaction) activity at a serotonin-2 (5-HT₂) receptor. In some embodiments, the 5-HT₂ agent acts as a receptor modulator, wherein the 5-HT₂ agent alters the function of a receptor to which it binds, the alteration including any of activating the activity of the receptor, inhibiting the activity of the receptor, and activating or inhibiting the activity of the receptor, depending on the dose administered.

Many compounds that possess affinity for one serotonin receptor subfamily can also bind to serotonin receptors of other subfamilies, or possess affinity for receptors of the serotonin receptor system as a whole. Accordingly, in some embodiments, the 5-HT₂ agent is a compound that also has affinity for another serotonin receptor. In embodiments, the 5-HT₂ agent is a phenethylamine or tryptamine disclosed herein as a serotonin receptor agent, which may also have affinity for other serotonin receptor subfamilies in addition to the 5-HT₂ receptor. However, 5-HT₂ agents can also be differentiated from other serotonin receptor agents, for example according to potency at the 5-HT₂ receptor (e.g., measured in absolute terms, such as Kᵢ), or by their selectivity (e.g., as determined by comparing the Kᵢ values to calculate selectivity over a given receptor) for the 5-HT₂ receptor subfamily, or a specific 5-HT₂ receptor subtype, relative to another receptor system, or relative to another serotonin receptor subfamily (e.g., 5-HT₇).

In some embodiments, the 5-HT₂ agent is a “selective agent,” which may refer to an agent that binds to the 5-HT₂ receptor system more selectively than receptors not part of the 5-HT₂ receptor system. In some embodiments, such selectivity (also termed “specificity” herein), is expressed as a ratio greater than 1.0. In some embodiments, selectivity (as measured via Kᵢ) for the 5-HT₂ receptor system is at least 1.1:1, 1.5:1, 2:1, 5:1, 10:1, 15:1, 25:1, 50:1, 100:1, etc., as compared to receptors not part of the 5-HT₂ receptor system. In embodiments, selectivity (as Kᵢ) for the 5-HT₇ receptor system is greater than 1.0, such as but not limited to 1.1:1, 1.5:1, 2:1, 5:1, 10:1, 15:1, 25:1, 50:1, 100:1, etc., as compared to 5-HT₁ receptors. In embodiments, selectivity (as Kᵢ) for the 5-HT₇ receptor system is greater than 1.0, such as but not limited to 1.1:1, 1.5:1, 2:1, 5:1, 10:1, 15:1, 25:1, 50:1, 100:1, etc., as compared to 5-HT₇ receptors.

In embodiments, a serotonin-2 agent is an agonist. Broadly, a “serotonin-2 receptor agonist” (or a “serotonin-2 agonist,” “5-HT₂ receptor agonist,” or “5-HT₂ agonist”) is a compound that initiates a biologic response when bound to a serotonin-2 receptor. A “serotonin-2 receptor” will include serotonin-2A, 2B, and 2C receptors. In embodiments, a 5-HT₂ agonist has a high specificity for 5-HT₂ receptors. In embodiments, a 5-HT₂ agonist has a low specificity for receptors not part of the serotonin-2 receptor system.

In some embodiments, a serotonin-2 agent is an antagonist. Broadly, a “serotonin-2 receptor antagonist” (or a “serotonin-2 antagonist,” “5-HT₂ receptor antagonist,” or “5-HT₂ antagonist”) is a compound that inhibits a biological response when bound to a serotonin-2 receptor. A “serotonin-2 receptor” will include serotonin-2A, 2B, and 2C receptors. In some embodiments, the 5-HT₂ antagonist has a high specificity for 5-HT₂ receptors. In some embodiments, the 5-HT₂ antagonist has a low specificity for receptors not part of the serotonin-2 receptor system.

In some embodiments, a 5-HT₂ agent is an inverse agonist. Broadly, a “5-HT₂ receptor inverse agonist” (or a “5-HT₂ inverse agonist,” “5-HT₂ receptor inverse agonist,” or “5-HT₂ inverse agonist” is a compound that, when bound to a 5-HT₂ receptor, lowers the 5-HT₂ receptor's constitutive activity to a level below its basal state. A “5-HT₂ receptor” will include serotonin-2A, 2B, and 2C receptors. In embodiments, the 5-HT₂ inverse agonist has a high specificity for 5-HT₂ receptors. In embodiments, the 5-HT₂ inverse agonist has a low specificity for receptors not part of the 5-HT₂ receptor system.

In some embodiments, a “5-HT₂ agonist” is selective for 5-HT_{2A} or 5-HT_{2C} over 5-HT_{2B}. In some embodiments, a 5-HT₂ agonist is selective for 5-HT_{2A} and 5-HT_{2C} over 5-HT_{2B}. In some embodiments, a 5-HT₂ agonist is selective for 5-HT_{2A} over 5-HT_{2B} or 5-HT_{2C}. In some embodiments, a 5-HT₂ agonist is selective for 5-HT_{2A} over 5-HT_{2B} and 5-HT_{2C}. In some embodiments, a 5-HT₂ agonist is selective for 5-HT_{2C} over 5-HT_{2A} or 5-HT_{2B}. In some embodiments, a 5-HT₂ agonist is selective for 5-HT_{2C} over 5-HT_{2A} and 5-HT_{2B}. In some embodiments, a 5-HT₂ agonist is selective for 5-HT_{2B} over 5-HT_{2A} or 5-HT_{2C}. In some embodiments, a 5-HT₂ agonist is selective for 5-HT_{2B} over 5-HT_{2A} and 5-HT_{2C}. In some embodiments, a 5-HT₂ agonist is selective for any one or more of 5-HT_{2A}, 5-HT_{2B}, and 5-HT_{2C} over 5-HT₇. In some embodiments, a 5-HT₂ agonist is selective for all of 5-HT_{2A}, 5-HT_{2B}, and 5-HT_{2C} over 5-HT₇. In some embodiments, a 5-HT₂ agonist is selective for 5-HT_{2A} over 5-HT₇.

In some embodiments, the disclosed 5-HT_{2A} agonists and other 5-HT_{2A} agents will not cause psychedelic effects when administered as part of disclosed entactogenic combinations and pharmaceutical compositions. In some embodiments, the disclosed 5-HT_{2A} agonists and other 5-HT_{2A} agents will not cause psychedelic effects when administered as part of disclosed entactogenic combinations and pharmaceutical compositions, when administered at doses or in methods which would otherwise cause psychedelic effects, were such agents administered alone.

Serotonin receptor agents generally include tryptamines and phenethylamines. In embodiments, an exemplary 5-HT₂ agent is a tryptamine. In embodiments, an exemplary 5-HT₂ agent is a phenethylamine.

“Tryptamines” are as readily understood by those in the art, and examples of tryptamines that are useful in the practice of the invention include, but are not limited to: baeocystin, norbaeocystin, tryptamine, 4-hydroxytryptamine (or, for shorthand, “4-hydroxyl” wherein the ultimate “T” stands for “tryptamine”), N-methylT, N-ethylT, N-propylT, N-isopropylT, N-allylT, N,N-dimethylT, N,N-diethylT, N,N-dipropylT, N,N-diisopropylT, N,N-diallylT, N-methyl-N-ethylT, N-methyl-N-propylT, N-methyl-N-isopropylT, N-methyl-N-allylT, N-ethyl-N-propylT, N-ethyl-N-isopropylT, N-ethyl-N-allylT, N-propyl-N-isopropylT, N-propyl-N-allylT, N-isopropyl-N-allylT, 4-hydroxy-T, 4-hydroxy-N-methylT, 4-hydroxy-N-ethylT, 4-hydroxy-N-propylT, 4-hydroxy-N-isopropylT, 4-hydroxy-N-allylT, 4-hydroxy-N,N-dimethylT, 4-hydroxy-N,N-diethylT, 4-hydroxy-N,N-dipropylT, 4-hydroxy-N,N-diisopropylT, 4-hydroxy-N,N-diallylT, 4-hydroxy-N-methyl-N-ethylT, 4-hydroxy-N-methyl-N-propylT, 4-hydroxy-N-methyl-N-isopropyl-T, 4-hydroxy-N-methyl-N-allylT, 4-hydroxy-N-ethyl-N-propylT, 4-hydroxy-N-ethyl-N-isopropylT, 4-hydroxy-N-ethyl-N-allylT, 4-hydroxy-N-propyl-N-isopropylT, 4-hydroxy-N-propyl-N-allylT, 4-hydroxy-N-isopropyl-N-allylT, 5-hydroxy-T, 5-hydroxy-N-methylT, 5-hydroxy-N-ethylT, 5-hydroxy-N-propylT, 5-hydroxy-N-isopropylT, 5-hydroxy-N-allylT, 5-hydroxy-N,N-dimethylT, 5-hydroxy-N,N-diethylT, 5-hydroxy-N,N-dipropylT, 5-hydroxy-N,N-diisopropylT, 5-hydroxy-N,N-diallylT, 5-hydroxy-N-methyl-N-ethylT, 5-hydroxy-N-methyl-N-propyl-T, 5-hydroxy-N-methyl-N-isopropylT, 5-hydroxy-N-methyl-N-allylT, 5-hydroxy-N-ethyl-N-propylT, 5-hydroxy-N-ethyl-N-isopropyl-T, 5-hydroxy-N-ethyl-N-allylT, 5-hydroxy-N-propyl-N-isopropylT, 5-hydroxy-N-propyl-N-allylT, 5-hydroxy-N-isopropyl-N-allylT, 4-methoxy-T, 4-methoxy-N-methylT, 4-methoxy-N-ethylT, 4-methoxy-N-propylT, 4-methoxy-N-isopropylT, 4-methoxy-N-allylT, 4-methoxy-N,N-dimethylT, 4-methoxy-N,N-diethylT, 4-methoxy-N,N-dipropyl-T, 4-methoxy-N,N-diisopropylT, 4-methoxy-N,N-diallylT, 4-methoxy-N-methyl-N-ethylT, 4-methoxy-N-methyl-N-propylT, 4-methoxy-N-methyl-N-isopropylT, 4-methoxy-N-methyl-N-allylT, 4-methoxy-N-ethyl-N-propylT, 4-methoxy-N-ethyl-N-isopropylT, 4-methoxy-N-ethyl-N-allylT, 4-methoxy-N-propyl-N-isopropylT, 4-methoxy-N-propyl-N-allylT, 4-methoxy-N-isopropyl-N-allylT, 5-methoxy-T, 5-methoxy-N-methylT, 5-methoxy-N-ethylT, 5-methoxy-N-propylT, 5-methoxy-N-isopropylT, 5-methoxy-N-allylT, 5-methoxy-N,N-dimethylT, 5-methoxy-N,N-diethylT, 5-methoxy-N,N-dipropylT, 5-methoxy-N,N-diisopropylT, 5-methoxy-N,N-diallylT, 5-methoxy-N-methyl-N-ethylT, 5-methoxy-N-methyl-N-propylT, 5-methoxy-N-methyl-N-isopropyl-T, 5-methoxy-N-methyl-N-allylT, 5-methoxy-N-ethyl-N-propylT, 5-methoxy-N-ethyl-N-isopropylT, 5-methoxy-N-ethyl-N-allylT, 5-methoxy-N-propyl-N-isopropylT, 5-methoxy-N-propyl-N-allylT, 5-methoxy-N-isopropyl-N-allylT, 4-acetoxy-T, 4-acetoxy-N-methylT, 4-acetoxy-N-ethylT, 4-acetoxy-N-propylT, 4-acetoxy-N-isopropylT, 4-acetoxy-N-allylT, 4-acetoxy-N,N-dimethylT, 4-acetoxy-N,N-diethylT, 4-acetoxy-N,N-dipropylT, 4-acetoxy-N,N-diisopropylT, 4-acetoxy-N,N-diallylT, 4-acetoxy-N-methyl-N-ethylT, 4-acetoxy-N-methyl-N-propylT, 4-acetoxy-N-methyl-N-isopropyl-T, 4-acetoxy-N-methyl-N-ally)-T, 4-acetoxy-N-ethyl-N-propylT, 4-acetoxy-N-ethyl-N-isopropyl-T, 4-acetoxy-N-ethyl-N-allylT, 4-acetoxy-N-propyl-N-isopropylT, 4-acetoxy-N-propyl-N-allylT, 4-acetoxy-N-isopropyl-N-allylT, 5-acetoxy-T, 5-acetoxy-N-methylT, 5-acetoxy-N-ethylT, 5-acetoxy-N-propylT, 5-acetoxy-N-isopropylT, 5-acetoxy-N-allylT, 5-acetoxy-N,N-dimethylT, 5-acetoxy-N,N-diethylT, 5-acetoxy-N,N-dipropylT, 5-acetoxy-N,N-diisopropylT, 5-acetoxy-N,N-diallylT, 5-acetoxy-N-methyl-N-ethylT, 5-acetoxy-N-methyl-N-propylT, 5-acetoxy-N-methyl-N-isopropyl-T, 5-acetoxy-N-methyl-N-allylT, 5-acetoxy-N-ethyl-N-propylT, 5-acetoxy-N-ethyl-N-isopropylT, 5-acetoxy-N-ethyl-N-allylT, 5-acetoxy-N-propyl-N-isopropylT, 5-acetoxy-N-propyl-N-allylT, 5-acetoxy-N-isopropyl-N-allylT, α-methylT, N-ethyl-N-isopropylT, N-methyl-N-butyl-T, 2,α-dimethylT, α-N-dimethylT, α-methyl-N,N-dimethyl-T, α-ethylT, 2-methyl-N,N-dimethylT, 2-methyl-N,N-diethyl-T, 1-methylpsilocin, 5-methoxy-α-methylT, ibogaine, harmaline, 7-methoxy-1-methyl-1,2,3,4-tetrahydro-b-carboline (tetrahydroharmine), N,N-diethyl-D-lysergamide (LSD), 6-ally-N,N-diethyl-norlysergic acid (6-allyl-N,N-diethyl-norlysergic acid), 9,10-didehydro-N,N,6-triethylergoline-8b-carboxamide (6, N,N-triethyl-norlysergic acid), 9,10-didehydro-6-propyl-N,N-diethylergoline-8b-carboxamide (6-propyl-norlysergic acid), 4-OH-DALT, 5-Br-DALT, 5-MeO-DALT, 4-AcO-DALT, 5-F-DALT, 5-F-2-Me-DALT, 7-Et-DALT, DALT, 2-Ph-DALT, 5-MeO-2-Me-DALT, other tryptamine compounds, or a pharmaceutically acceptable salt, hydrate, solvate, precursor, prodrug, metabolite, derivative, analog, isotopolog, variant, tautomer, isomer, or stereoisomer (including pure or substantially pure individual enantiomers and enantiomerically enriched mixtures having any enantiomeric excess greater than 0%) thereof, or a combination thereof (and including all amorphous and polymorphic forms).

In embodiments, where a compound is a tryptamine, its ring-substituted derivatives as known in the art are also useful in the practice of the invention. For example, where a compound is DALT, ring-substituted derivatives (among others, including other known derivatives) include, e.g., its 2-phenyl-, 4-acetoxy-, 4-hydroxy-, 5-methoxy-, 5-methoxy-2-methyl-, 5-fluoro-, 5-fluoro-2-methyl-, 5-bromo-, and 7-ethyl-derivatives (Klein, 2018). Where a compound is another tryptamine, similar ring-substituted derivatives such as these described above (and others) will be readily appreciated to those of ordinary skill.

Those of skill will readily appreciate how to determine whether compounds are, e.g., 5-HT₂ and/or 5-HT₇ agents, agonists, and/or antagonists, as well as their selectivity for one or more receptor systems. For example, using data available in Klein 2018, the above derivatives in rank order of relative affinity for 5-HT₇ are: 4-OH-DALT, 5-Br-DALT, 5-MeO-DALT, 4-AcO-DALT, 5-F-DALT, 5-F-2-Me-DALT. In rank order of relative affinity for 5-HT₂ are: 7-Et-DALT, DALT, 2-Ph-DALT, 5-MeO-2-Me-DALT. One of skill will appreciate how to perform such an analysis and ranking of any compounds taught or suggested herein, whether based on already public data or data that can be generated based on the teachings herein and general skill in the art.

Yet other psychoactive tryptamines that are contemplated for use in the practice of the invention include: 6-allyl-N,N-diethyl-norlysergamide (AL-LAD), N,N-dibutyltryptamine (“N,N-dibutyltrypT,” using the shorthand described elsewhere; DBT), α,N-dimethylT (α,N-DMT), 6, N,N-triethylnorlysergamide (ETH-LAD), 3,4-dihydro-7-methoxy-1-methylcarboline (Harmaline), 7-methoxy-1-methylcarboline (Harmine), N,N-dibutyl-4-hydroxyT (4-HO-DBT), N,N-diethyl-4-hydroxyT (4-HO-DET), N,N-diisopropyl-4-hydroxyT (4-HO-DiPT), N,N-dimethyl-4-hydroxyT (4-HO-DMT), N,N-dimethyl-5-hydroxyT (5-HO-DMT, bufotenine), N,N-dipropyl-4-hydroxyT (4-HO-DPT), N-ethyl-4-hydroxy-N-methylT (4-HO-MET), 4-hydroxy-N-isopropyl-N-methylT (4-HO-MiPT), 4-hydroxy-N,N-tetramethylene-T (4-HO-pyr-T), 12-methoxyibogamine (Ibogaine), N-butyl-N-methylT (MBT), N,N-diisopropyl-4,5-methylenedioxyT (4,5-MDO-DiPT), N,N-diisopropyl-5,6-methylene-dioxyT (5,6-MDO-DiPT), N,N-dimethyl-4,5-methylenedioxyT (4,5-MDO-DMT), N,N-dimethyl-5,6-methylene-dioxyT (5,6-MDO-DMT), N-isopropyl-N-methyl-5,6-methylenedioxyT (5,6-MDO-MiPT), N,N-diethyl-2-methylT (2-Me-DET), 2,N,N-trimethylT (2-Me-DMT), N-acetyl-5-methoxyT (melatonin), N,N-diethyl-5-methoxyT (5-MeO-DET), N,N-diisopropyl-5-methoxyT (5-MeO-DiPT), N-isopropyl-4-methoxy-N-methylT (4-MeO-MiPT), N-isopropyl-5-methoxy-N-methylT (5-MeO-MiPT), 5,6-dimethoxy-N-isopropyl-N-methylT (5,6-MeO-MiPT), 5-methoxy-N,N-tetramethyleneT (5-MeO-pyr-T), 6-methoxy-1-methyl-1,2,3,4-tetra-hydrocarboline (6-MeO-THH), 5-methoxy-2,N,N-trimethylT (5-MeO-TMT), N,N-dimethyl-5-methylthioT (5-MeS-DMT), N-isopropyl-N-methyl-T (MiPT), 6-propylnorlysergamide (PRO-LAD), N,N-tetramethyleneT (pyr-T), Tryptamine (T), 7-methoxy-1-methyl-1,2,3,4-tetrahydrocarboline (Tetrahydroharmine), or α,N-dimethyl-5-methoxyT (α,N,O-TMS), or a pharmaceutically acceptable isomer, salt, ester, hydrate, solvate, polymorph, prodrug, isotopolog, or metabolite thereof, or a combination thereof. See Shulgin and Shulgin, TIHKAL: The Continuation, Transform Press (1997), which is incorporated by reference in its entirety herein. Additional examples of psychedelic tryptamines can be found in numerous references known to those of skill (e.g., Araujo 2015).

It will be readily appreciated that while some psychoactive tryptamines of the invention can be obtained from fungal sources, other disclosed tryptamine compounds can be obtained from numerous other plant and botanical sources. For example, N,N-DMT may be extracted from, among other sources, Phalaris, Delosperma, Acacia, Desmodium, Mimosa, Virola, and Psychotria spp.

In embodiments, a tryptamine will be a substituted tryptamine having the structure below, wherein R^{N1}, R^{N2}, R^{α}, R^{β}, R², R⁴, R⁵, R⁶, and R⁷ will be as taught herein and as generally understood in the art:

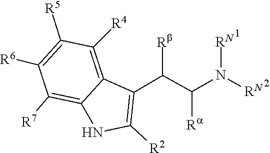

For example, in some embodiments, R^{N1}, R^{N2}, R^{α}, R^{β}, R², R⁴, R⁵, R⁶, and R⁷ are independently hydrogen, deuterium, halogen, hydroxy, methoxy, phosphoryloxy, C₁-C₅ alkyl, C₂-C₈ alkenyl, C₂-C₈ alkynyl, C₃-C₈ cycloalkyl (independently or ring closed with the nitrogen), C₃-C₈ cycloalkenyl (independently or ring closed with the nitrogen), aryl, or heterocyclyl, any of which are optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate, —OP(O)(OH)₂, —OC(O)H, —OSO₂OH, —OC(O)NH₂, and —SONH. In addition, tryptamines useful in the practice of the invention also necessarily include any salts, isomers (including structural isomers and stereoisomers, such as enantiomers), precursors, prodrugs, metabolites, derivatives, analogs, isotopologs, or variants thereof, or a combination thereof.

In embodiments, a tryptamine will be a tryptamine N-substituted with any of a methy, ethyl, propyl, isopropyl, or allyl group, and in embodiments may include tryptamine, N-methyltryptamine, N-ethyltryptamine, N-propyltryptamine, N-isopropyltryptamine, N-allyltryptamine, N,N-dimethyltryptamine, N,N-diethyltryptamine, N,N-dipropyltryptamine, N,N-diisopropyltryptamine, N,N-diallyltryptamine, N-methyl-N-ethyltryptamine, N-methyl-N-propyltryptamine, N-methyl-N-isopropyltryptamine, N-methyl-N-allyltryptamine, N-ethyl-N-propyltryptamine, N-ethyl-N-isopropyltryptamine, N-ethyl-N-allyltryptamine, N-propyl-N-isopropyltryptamine, N-propyl-N-allyltryptamine, and N-isopropyl-N-allyltryptamine.

In some embodiments, a tryptamine of the invention will be a “complex tryptamine” or other indolamine and include such non-limiting examples as (as the chemical classes are ordinarily known in the art, and including at least such exemplary compounds as generally known in the art, as well as analogs and derivatives thereof) ergolines and ergoline alkaloids (including ergoline derivatives such as lysergamides, ergopeptines or ergopeptides, and clavines; and including such synthetic ergoline derivatives as cabergoline, pergolide, and lisuride), natural and synthetic ibogaine alkaloids, and other plant alkaloids including harmine, harmane, norharmine, harmaline, perlolyrine, harmol, and other beta-carbolines, e.g., from Banisteriopsis caapi and Peganum harmala, or other monoamine oxidase inhibitor (MAOI), such as will enhance the pharmaceutical efficacy of a tryptamine when used in combination therewith in the practice of the invention. Accordingly, in some embodiments, and in some preferred embodiments wherein a tryptamine or complex tryptamine is taken orally (especially where such tryptamine is not orally active otherwise), a MAOI will be an additional active agent in the composition or therapeutic combination, in such dosage to provide oral activity of the tryptamine or complex tryptamine, as known to one of skill.

Non-limiting examples of “phenethylamines,” as would be readily apparent to one of skill, include: α-ethyl-3,4,5-trimethoxyphenethylamine (or for shorthand, “α-ethyl-3,4,5-trimethoxyPEA,” where “PEA” refers to “phenethylamine”; AEM), 4-allyloxy-3,5-dimethoxyPEA (AL), 2,5-dimethoxy-4-methyl-thioamphetamine (or, for shorthand, “2,5-dimethoxy-4-methyl-thioA,” where “A” stands for “amphetamine”; ALEPH), 2,5-dimethoxy-4-ethylthioA (ALEPH-2), 2,5-dimethoxy-4-isopropylthioA (ALEPH-4), 2,5-dimethoxy-4-phenylthioA (ALEPH-6), 2,5-dimethoxy-4-propylthioA (ALEPH-7), 2,5-dimethoxy-α-ethyl-4-methylPEA (ARIADNE), 3,4-diethoxy-5-methoxyPEA (ASB), 4-butoxy-3,5-dimethoxy-PEA (B), 2,5-dimethoxy-4,N-dimethylA (BEATRICE), 2,5-bismethylthio-4-methylA (BIS-TOM), 4-bromo-2,5,ß-trimethoxyPEA (BOB), 2,5,ß-trimethoxy-4-methylPEA (BOD), ß-methoxy-3,4-methylenedioxyPEA (BOH), 2,5-dimethoxy-ß-hydroxy-4-methylPEA (BOND), 3,4,5, ß-tetramethoxyPEA (BOM), 4-bromo-3,5-dimethoxyA (4-Br-3,5-DMA), 2-bromo-4,5-methylenedioxyA (2-Br-4,5-MDA), 4-bromo-2,5-dimethoxy-PEA (2C-B), 2-(4-bromo-2,3,6,7-tetrahydrofuro[2,3-f][1]benzofuran-8-yl)ethanamine (2C-B-FLY), 4-benzyloxy-3,5-dimethoxyA (3C-BZ), 4-chloro-2,5-dimethoxy-PEA (2C-C), 2,5-dimethoxy-4-methyl-PEA (2C-D), 2,5-dimethoxy-4-ethyl-PEA (2C-E), 3,5-dimethoxy-4-ethoxyA (3C-E), 2,5-dimethoxy-4-fluoroPEA (2C-F), 2,5-dimethoxy-3,4-dimethylPEA (2C-G), 2,5-dimethoxy-3,4-trimethylenePEA (2C-G-3), 2,5-dimethoxy-3,4-tetramethylene-PEA (2C-G-4), 3,4-norbornyl-2,5-dimethoxyPEA (2C-G-5), 1,4-dimethoxynaphthyl-2-ethylamine (2C-G-N), 2,5-dimethoxyPEA (2C-H), 4-iodo-2,5-dimethoxyPEA (2C-I), 2,5-dimethoxy-4-nitro-PEA (2C-N), 2,5-dimethoxy-4-isopropoxyPEA (2C-O-4), 2,5-dimethoxy-4-propyl PEA (2C-P), 4-cyclopropylmethoxy-3,5-dimethoxyPEA (CPM), 2,5-dimethoxy-4-methylselenoPEA (2C-SE), 2,5-dimethoxy-4-methylthioPEA (2C-T), 2,5-dimethoxy-4-ethylthioPEA (2C-T-2), 2,5-dimethoxy-4-isopropylthioPEA (2C-T-4), 2,6-dimethoxy-4-isopropylthioPEA (psi-2C-T-4), 2,5-dimethoxy-4-propylthioPEA (2C-T-7), 4-cyclopropylmethylthio-2,5-dimethoxyPEA (2C-T-8), 4-(t)-butylthio-2,5-dimethoxy-PEA (2C-T-9), 2,5-dimethoxy-4-(2-methoxyethylthio) PEA (2C-T-13), 4-cyclopropylthio-2,5-dimethoxyPEA (2C-T-15), 4-(s)-butylthio-2,5-dimethoxyPEA (2C-T-17), 2,5-dimethoxy-4-(2-fluoroethylthio)PEA (2C-T-21), 3,5-dimethoxy-4-trideuteromethylPEA (4-D), ß,ß-dideutero-3,4,5-trimethoxyPEA (ß-D), 3,5-dimethoxy-4-methyl-PEA (DESOXY), 2,4-dimethoxyA (2,4-DMA), 2,5-dimethoxyA (2,5-DMA), 3,4-dimethoxyA (3,4-DMA), 2-(2,5-dimethoxy-4-methylphenyl)cyclopropylamine (DMCPA), 3,4-dimethoxy-ß-hydroxyPEA (DME), 2,5-dimethoxy-3,4-methylenedioxyA (DMMDA), 2,3-dimethoxy-4,5-methylenedioxyA (DMMDA-2), 3,4-dimethoxyPEA (DMPEA), 4-amyl-2,5-dimethoxyA (DOAM), 4-bromo-2,5-dimethoxyA (DOB), 4-butyl-2,5-dimethoxyA (DOBU), 4-chloro-2,5-dimethoxyA (DOC), 2,5-dimethoxy-4-(2-fluoroethyl)A (DOEF), 2,5-dimethoxy-4-ethylA (DOET), 4-iodo-2,5-dimethoxyA (DOI), 2,5-dimethoxy-4-methylA (DOM (STP)), 2,6-dimethoxy-4-methylA (psi-DOM), 2,5-dimethoxy-4-nitroA (DON), 2,5-dimethoxy-4-propylA (DOPR), 3,5-dimethoxy-4-ethoxyPEA (E), 2,4,5-triethoxyA (EEE), 2,4-diethoxy-5-methoxyA (EEM), 2,5-diethoxy-4-methoxyA (EME), 4,5-dimethoxy-2-ethoxyA (EMM), 2-ethylamino-1-(3,4-methylenedioxyphenyl)butane (ETHYL-J), 2-ethylamino-1-(3,4-methylene-dioxyphenyl)pentane (ETHYL-K), 6-(2-aminopropyl)-5-methoxy-2-methyl-2,3-dihydrobenzofuran (F-2), 6-(2-aminopropyl)-2,2-dimethyl-5-methoxy-2,3-dihydrobenzofuran (F-22), N-hydroxy-N-methyl-3,4-methylenedioxyA (FLEA), 2,5-dimethoxy-3,4-(trimethylene)A (G-3), 2,5-dimethoxy-3,4-(tetramethylene)A (G-4), 3,6-dimethoxy-4-(2-aminopropyl)benzonorbornane (G-5), 2,5-dimethoxy-3,4-dimethylA (GANESHA), 1,4-dimethoxynaphthyl-2-isopropylamine (G-N), 2,5-dimethoxy-4-ethylthio-N-hydroxyPEA (HOT-2), 2,5-dimethoxy-N-hydroxy-4-(n)-propylthioPEA (HOT-7), 4-(s)-butylthio-2,5-dimethoxy-N-hydroxyPEA (HOT-17), 2,5-dimethoxy-N,N-dimethyl-4-iodoA (IDNNA), 2,3,4-trimethoxyPEA (IM), 3,5-dimethoxy-4-isopropoxyPEA (IP), 5-ethoxy-2-methoxy-4-methylA (IRIS), 2-amino-1-(3,4-methylene-dioxyphenyl)butane (J, BDB), 3-methoxy-4,5-methylenedioxyPEA (LOPHOPHINE), 3,4,5-trimethoxyPEA (M), 4-methoxyA (4-MA, PMA), 2,N-dimethyl-4,5-methylenedioxyA (MADAM-6), 3,5-dimethoxy-4-methallyloxyPEA (MAL), 3,4-methylenedioxyA (MDA), N-allyl-3,4-methylenedioxyA (MDAL), N-butyl-3,4-methylenedioxyA (MDBU), N-benzyl-3,4-methylenedioxyA (MDBZ), N-cyclopropylmethyl-3,4-methylenedioxyA (MDCPM), N,N-dimethyl-3,4-methylenedioxyA (MDDM), N-ethyl-3,4-methylenedioxyA (MDE), N-(2-hydroxyethyl)-3,4-methylenedioxyA (MDHOET), N-isopropyl-3,4-methylenedioxyA (MDIP), N-methyl-3,4-methylenedioxyA (MDMA), 3,4-ethylenedioxy-N-methylA (MDMC), N-methoxy-3,4-methylene-dioxyA (MDMEO), N-(2-methoxyethyl)-3,4-methylenedioxyA (MDMEOET), 3,4-methylenedioxy-α,α,N-trimethylPEA (MDMP), N-hydroxy-3,4-methylenedioxyA (MDOH), 3,4-methylenedioxyPEA (MDPEA), α,α-dimethyl-3,4-methylenedioxyPEA (MDPH), 3,4-methylenedioxy-N-propargylA (MDPL), 3,4-methylene-dioxy-N-propyl-A (MDPR), 3,4-dimethoxy-5-ethoxyPEA (ME), 4,5-ethylenedioxy-3-methoxyA (MEDA), 4,5-diethoxy-2-methoxyA (MEE), 2,5-dimethoxy-4-ethoxyA (MEM), 4-ethoxy-3-methoxyPEA (MEPEA), 5-bromo-2,4-dimethoxyA (META-DOB), 2,4-dimethoxy-5-methylthioA (META-DOT), 2,5-dimethoxy-N-methylA (METHYL-DMA), 4-bromo-2,5-dimethoxy-N-methylA (METHYL-DOB), 2-methylamino-1-(3,4-methylenedioxyphenyl)butane (METHYL-J, MBDB), 2-methylamino-1-(3,4-methylenedioxyphenyl)pentane (METHYL-K), 4-methoxy-N-methylA (METHYL-MA, PMMA), 2-methoxy-N-methyl-4,5-methylenedioxyA (METHYL-MMDA-2), 3-methoxy-4,5-methylenedioxyA (MM DA), 2-methoxy-4,5-methylenedioxyA (MMDA-2), 2-methoxy-3,4-methylenedioxyA (MMDA-3a), 4-methoxy-2,3-methylenedioxyA (MMDA-3b), 2,4-dimethoxy-5-ethoxyA (MME), 3,4-dimethoxy-5-(n)-propoxyPEA (MP), 2,5-dimethoxy-4-(n)-propoxyA (MPM), 4,5-dimethoxy-2-methylthioA (ORTHO-DOT), 3,5-dimethoxy-4-propoxyPEA (P), 3,5-dimethoxy-4-phenethyloxyPEA (PE), PEA (PEA), 3,5-dimethoxy-4-(2-propynyloxy)PEA (PROPYNYL), 3,5-diethoxy-4-methoxyPEA (SB), 2,3,4,5-TetramethoxyA (TA), 4-ethoxy-3-ethylthio-5-methoxyPEA (3-TASB), 3-ethoxy-4-ethylthio-5-methoxyPEA (4-TASB), 3,4-diethoxy-5-methylthioPEA (5-TASB), 4-(n)-butylthio-3,5-dimethoxyPEA (TB), 4-ethoxy-5-methoxy-3-methylthioPEA (3-TE), 3,5-dimethoxy-4-ethylthioPEA (TE, 4-TE), 3,4-dimethoxy-2-methylthioPEA (2-TIM), 2,4-dimethoxy-3-methylthioPEA (3-TIM), 2,3-dimethoxy-4-methylthioPEA (4-TIM), 3,4-dimethoxy-5-methylthioPEA (3-TIM), 3,5-dimethoxy-4-methylthioPEA (4-TM), 3,4,5-trimethoxyA (TMA), 2,4,5-trimethoxyA (TMA-2), 2,3,4-trimethoxyA (TMA-3), 2,3,5-trimethoxyA (TMA-4), 2,3,6-trimethoxyA (TMA-5), 2,4,6-trimethoxyA (TMA-6), 4,5-dimethoxy-3-ethylthioPEA (3-TME), 3-ethoxy-5-methoxy-4-methylthioPEA (4-TME), 3-ethoxy-4-methoxy-5-methylthioPEA (5-TME), 3,4-methylenedioxy-2-methylthioA (2T-MMDA-3a), 2-methoxy-4,5-methylene-thiooxyA (4T-MMDA-2), 2,4,5-trimethoxyPEA (TMPEA), 4-ethyl-5-methoxy-2-methylthioA (2-TOET), 4-ethyl-2-methoxy-5-methylthioA (5-TOET), 5-methoxy-4-methyl-2-methylthioA (2-TOM), 2-methoxy-4-methyl-5-methylthioA (5-TOM), 2-methoxy-4-methyl-5-methylsulfinylA (TOMSO), 3,5-dimethoxy-4-propylthioPEA (TP), 3,4,5-triethoxyPEA (TRIS), 3-ethoxy-5-ethylthio-4-methoxyPEA (3-TSB), 3,5-diethoxy-4-methylthio PEA (4-TSB), 3,4-diethoxy-5-ethylthioPEA (3-T-TRIS), 3,5-diethoxy-4-ethylthioPEA (4-T-TRIS), (R)-2,5-dimethoxy-4-iodoA (R-DOI), or a pharmaceutically acceptable isomer, salt, ester, hydrate, polymorph, solvate, prodrug, isotopolog, or metabolite thereof, or a combination thereof. See Shulgin and Shulgin, PIHKAL: A Chemical Love Story, Transform Press (1994), incorporated by reference in its entirety herein.

In some embodiments, a phenethylamine useful in the practice of the invention will be a substituted phenethylamine derived from the “2C” or “2C-x” family known as an “NBOMe,” “25-NB,” or “25x-NBx” compound. Such compounds, for example, may act as extremely potent and relatively selective 5-HT_{2A} agonists. Without being bound by theory, and understanding that exceptions exist, some of which are listed below, NBOMe compounds are generally N-benzylphenethylamines (although the phenyl ring of the N-benzyl group may be replaced by other heterocycles such as thiophene, pyridine, furan, tetrahydrofuran, benzodioxole or naphthalene, among others) with methoxy groups at the 2 and 5 positions of the phenyl ring, a substitution such as a halogen or alkyl group at the 4 position of the phenyl ring, and a methoxy or other substitution (e.g., hydroxyl, fluoro) at the 2 position of the N-benzyl ring. Other substitution patterns also may be present (e.g., NBOMe-mescaline, 25G-NBOMe, 2CBFly-NBOMe, and 25C-NB3OMe). NBOMe compounds generally differ from the 2C series by the presence of the N-benzyl moiety. In some compounds, an alpha-methyl group is present (making the compound an N-benzyl amphetamine); this typically reduces potency and activity. In some compounds, a side chain methyl group is cyclised back to the ring (e.g., 2CBCB-NBOMe) or links the two alpha positions (e.g., DMBMPP), which may improve selectivity for the 5-HT_{2A} receptor subtype.

Non-limiting examples of “NBOMe compounds” useful in the practice of the invention include: N-benzyl-1-(2,5-dimethoxy-4-bromophenyl)-2-aminoethane (25B-NB), N-benzyl-1-(2,5-dimethoxy-4-chlorophenyl)-2-aminoethane (25C-NB), N-benzyl-1-(2,5-dimethoxy-4-iodophenyl)-2-aminoethane (25I-NB), N-[(thiophen-2-yl)methyl]-1-(2,5-dimethoxy-4-iodophenyl)-2-aminoethane (25I-NMeTh), N-[(pyridin-2-yl) methyl]-1-(2,5-dimethoxy-4-bromophenyl)-2-aminoethane (25B-NMePyr), N-[(furan-2-yl)methyl]-1-(2,5-dimethoxy-4-iodophenyl)-2-aminoethane (25I-NMeFur), N-[(tetrahydrofuran-2-yl)methyl]-1-(2,5-dimethoxy-4-iodophenyl)-2-aminoethane (25I-NMeTHF), N-(2-fluorobenzyl)-1-(2,5-dimethoxy-4-bromophenyl)-2-aminoethane (25B-NBF), N-(2-hydroxybenzyl)-1-(2,5-dimethoxy-4-bromophenyl)-2-aminoethane (25B-NBOH), N-(2-methoxybenzyl)-1-(2,5-dimethoxy-4-bromophenyl)-2-aminoethane (25B-NBOMe), N-(2,3-dimethoxybenzyl)-1-(2,5-dimethoxy-4-bromophenyl)-2-aminoethane (25B-NB23DM), N-(2,5-dimethoxybenzyl)-1-(2,5-dimethoxy-4-bromophenyl)-2-aminoethane (25B-NB25DM), N-[(benzofuran-7-yl)methyl]-1-(2,5-dimethoxy-4-bromophenyl)-2-aminoethane (25B-NMe7BF), N-[(2,3-dihydrobenzofuran-7-yl)methyl]-1-(2,5-dimethoxy-4-bromophenyl)-2-aminoethane (25B-NMe7DHBF), N-[(benzothiophen-7-yl) methyl]-1-(2,5-dimethoxy-4-bromophenyl)-2-aminoethane (25B-NMe7BT), N-[(benzoxazol-7-yl)methyl]-1-(2,5-dimethoxy-4-bromophenyl)-2-aminoethane (25B-NMe7Box), N-[(indol-7-yl)methyl]-1-(2,5-dimethoxy-4-bromophenyl)-2-aminoethane (25B-NMe7Ind), N-[(indazol-7-yl)methyl]-1-(2,5-dimethoxy-4-bromophenyl)-2-aminoethane (25B-NMe7Indz), N-[(benzimidazol-7-yl)methyl]-1-(2,5-dimethoxy-4-bromophenyl)-2-aminoethane (25B-NMe7Bim), N-[(2-fluoroethoxy)benzyl]-1-(2,5-dimethoxy-4-bromophenyl)-2-aminoethane (FECIMBI-36), N-(2-methoxybenzyl)-1-(2,5-dimethoxy-4-bromophenyl)-2-aminopropane (DOB-NBOMe), N-(3-methoxybenzyl)-1-(2,5-dimethoxy-4-chlorophenyl)-2-aminoethane (25C-NB3OMe), N-(4-methoxybenzyl)-1-(2,5-dimethoxy-4-chlorophenyl)-2-aminoethane (25C-NB4OMe), N-(3,4,5-trimethoxybenzyl)-1-(2,5-dimethoxy-4-chlorophenyl)-2-aminoethane (C30-NBOMe), N-(2-fluorobenzyl)-1-(2,5-dimethoxy-4-chlorophenyl)-2-aminoethane (25C-NBF), N-(2-chlorobenzyl)-1-(2,5-dimethoxy-4-chlorophenyl)-2-aminoethane (25C-NBCI), N-(2-hydroxybenzyl)-1-(2,5-dimethoxy-4-chlorophenyl)-2-aminoethane (25C-NBOH), N-(2-methoxybenzyl)-1-(2,5-dimethoxy-4-chlorophenyl)-2-aminoethane (25C-NBOMe), N-(2-ethoxybenzyl)-1-(2,5-dimethoxy-4-chlorophenyl)-2-aminoethane (25C-NBOEt), N-(2-isopropoxybenzyl)-1-(2,5-dimethoxy-4-chlorophenyl)-2-aminoethane (25C-NBOiPr), N-(2-methoxybenzyl)-1-(2,5-dimethoxy-4-fluorophenyl)-2-aminoethane (25F-NBOMe), N-(2-hydroxybenzyl)-1-(2,5-dimethoxy-4-cyanophenyl)-2-aminoethane (25CN-NBOH), N-(2-methoxybenzyl)-1-(2,5-dimethoxy-4-cyanophenyl)-2-aminoethane (25CN-NBOMe), N-(2-methoxybenzyl)-1-(2,5-dimethoxy-4-methylphenyl)-2-aminoethane (25D-NBOMe), N-(2-hydroxybenzyl)-1-(2,5-dimethoxy-4-methylphenyl)-2-aminoethane (25D-NBOH), N-(2-methoxybenzyl)-1-(2,5-dimethoxy-4-ethylphenyl)-2-aminoethane (25E-NBOMe), N-(2-hydroxybenzyl)-1-(2,5-dimethoxy-4-ethylphenyl)-2-aminoethane (25E-NBOH), N-(2-methoxybenzyl)-1-(2,5-dimethoxy-3,4-dimethylphenyl)-2-aminoethane (25G-NBOMe), N-(2-methoxybenzyl)-1-(2,5-dimethoxyphenyl)-2-aminoethane (25H-NBOMe), N-(3,4-methylene-dioxybenzyl)-1-(2,5-dimethoxy-4-iodophenyl)-2-aminoethane (25I-NB34MD), N-(3-methoxybenzyl)-1-(2,5-dimethoxy-4-iodophenyl)-2-aminoethane (25I-NB3OMe), N-(4-methoxybenzyl)-1-(2,5-dimethoxy-4-iodophenyl)-2-aminoethane (25I-NB4OMe), N-(2-fluorobenzyl)-1-(2,5-dimethoxy-4-iodophenyl)-2-aminoethane (25I-NBF), N-(2-bromobenzyl)-1-(2,5-dimethoxy-4-iodophenyl)-2-aminoethane (25I-NBBr), N-[2-(trifluoromethyl) benzyl]-1-(2,5-dimethoxy-4-iodophenyl)-2-aminoethane (25I-NBTFM), N-(2,3-methylenedioxybenzyl)-1-(2,5-dimethoxy-4-iodophenyl)-2-aminoethane (25I-NBMD), N-(2,3-methylenedioxybenzyl)-1-(2,5-dimethoxy-4-bromophenyl)-2-aminoethane (25B-NBMD), N-(2,3-methylenedioxybenzyl)-1-(2,5-dimethoxy-4-chlorophenyl)-2-aminoethane (25C-NBMD), N-(2,3-methylenedioxybenzyl)-1-(2,5-dimethoxy-4-methylphenyl)-2-aminoethane (25D-NBMD), N-(2-hydroxybenzyl)-1-(2,5-dimethoxy-4-iodophenyl)-2-aminoethane (25I-NBOH), N-(2-methoxybenzyl)-1-(2,5-dimethoxy-4-iodophenyl)-2-aminoethane (25I-NBOMe), N-(2-methoxybenzyl)-1-(2,5-dimethoxy-4-iodophenyl)-2-aminopropane (DOI-NBOMe), N-[2-(hydroxymethyl)benzyl]-1-(2,5-dimethoxy-4-iodophenyl)-2-aminoethane (25I-NBMeOH), N-[2-(carbamoyl)benzyl]-1-(2,5-dimethoxy-4-iodophenyl)-2-aminoethane (25I-NBAm), N-[(2,3-dihydrobenzofuran-7-yl)methyl]-1-(2,5-dimethoxy-4-iodophenyl)-2-aminoethane (25I-NMe7DHBF), N-[(1-hydroxynaphthalen-2-yl)methyl]-1-(2,5-dimethoxy-4-iodophenyl)-2-aminoethane (25I-N2Nap1 OH), N-[(3-methoxythiophen-2-yl)methyl]-1-(2,5-dimethoxy-4-iodophenyl)-2-aminoethane (25I-N3MT2M), N-[(4-methoxythiophen-3-yl)methyl]-1-(2,5-dimethoxy-4-iodophenyl)-2-aminoethane (25I-N4MT3M), N-(2-methoxybenzyl)-1-(2,5-dimethoxy-4-isopropylphenyl)-2-aminoethane (25iP-NBOMe), N-(2-methoxybenzyl)-1-(2,5-dimethoxy-4-nitrophenyl)-2-aminoethane (25N-NBOMe), N-(2-methoxybenzyl)-1-(2,5-dimethoxy-4-propylphenyl)-2-aminoethane (25P-NBOMe), N-(2-hydroxybenzyl)-1-(2,5-dimethoxy-4-propylphenyl)-2-aminoethane (25P-NBOH), N-(2-methoxybenzyl)-1-[2,5-dimethoxy-4-(trifluoromethyl) phenyl]-2-aminoethane (25TFM-NBOMe), N-(2-methoxybenzyl)-1-[2,5-dimethoxy-4-(methylthio)phenyl]-2-amino-ethane (25T-NBOMe), N-(2-methoxybenzyl)-1-[2,5-dimethoxy-4-(ethylthio)phenyl]-2-aminoethane (25T2-NBOMe), N-(2-methoxybenzyl)-1-[2,5-dimethoxy-4-(isopropylthio) phenyl]-2-aminoethane (25T4-NBOMe), N-(2-methoxybenzyl)-1-[2,5-dimethoxy-4-(propylthio) phenyl]-2-aminoethane (25T7-NBOMe), N-(2-hydroxybenzyl)-1-[2,5-dimethoxy-4-(propylthio) phenyl]-2-aminoethane (25T7-NBOH), N-(2-methoxybenzyl)-1-(3,4,5-trimethoxyphenyl)-2-aminoethane (NBOMe-mescaline), N-(2-methoxybenzyl)-1-(3,5-dimethoxy-4-ethoxyphenyl)-2-aminoethane (NBOMe-escaline), N-(2-methoxybenzyl)-1-(3,4-methylenedioxyphenyl)-2-aminoethane (MDPEA-NBOMe), N-benzyl-1-(3,4-methylenedioxyphenyl)-2-aminopropane (MDBZ), N-(2-chlorobenzyl)-1-phenyl-2-aminopropane (Clobenzorex), N-(2-methoxybenzyl)-1-(4-ethylphenyl)-2-aminopropane (4-EA-NBOMe), N-(2-methoxybenzyl)-1-(benzofuran-5-yl)-2-aminopropane (5-APB-NBOMe), N-[1-(2-methoxyphenyl) ethyl]-2,5-dimethoxy-4-bromo-PEA (25B-N1POMe), 2-phenyl-2-[2-(2,5-dimethoxy-4-bromophenyl) ethylamino] acetonitrile (2C-B-AN), N-[(3-bromo-2,5-dimethoxy-bicyclo[4,2,0]octa-1,3,5-trien-7-yl) methyl]-1-(2-methoxyphenyl)methanamine (2CBCB-NBOMe), N-(2-methoxybenzyl)-1-(8-bromo-2,3,6,7-tetrahydrobenzo[1,2-b:4,5-b′]difuran-4-yl)-2-aminoethane (2CBFly-NBOMe), N-(2-hydroxybenzyl)-1-(8-bromobenzo[1,2-b:4,5-b′]difuran-4-yl)-2-aminoethane (2C-B-DragonFLY-NBOH), N-(2-ethoxy-5-chlorobenzyl)-1-(8-bromo-2,3,6,7-tetrahydrobenzo[1,2-b:4,5-b′]difuran-4-yl)-2-aminoethane (2C-B-FLY-NB2EtO5Cl), (S,S)-2-(2,5-dimethoxy-4-bromobenzyl)-6-(2-methoxyphenyl)piperidine (DMBMPP), 2-{[2-(4-bromo-2,5-dimethoxyphenyl)ethyl]amino}-1-(piperidin-1-yl)ethanone (25B-NAcPip), 1,3-dimethyl-7-{2-[1-(2,5-dimethoxy-4-chlorophenyl)propan-2-ylamino]ethyl}purine-2,6-dione (ZDCM-04), 3-[2-(2-methoxy-benzylamino)ethyl]-1H-quinazoline-2,4-dione (RH-34), N-(2-methoxybenzyl)-5-methoxyT (5MT-NBOMe), N-[(3-methoxythiophen-2-yl) methyl]-1-(2,5-dimethoxy-4-iodophenyl)-2-aminoethane (25I-N3MT2M), N-[(4-methoxy-thiophen-3-yl)methyl]-1-(2,5-dimethoxy-4-iodophenyl)-2-aminoethane (25I-N4MT3M), and N-(2-methoxybenzyl)-1-(2,5-dimethoxy-4-isopropylphenyl)-2-aminoethane (25iP-NBOMe).

In some embodiments, a phenethylamine useful in the practice of the invention will be an alpha-methyl substituted phenethylamine, i.e., a substituted amphetamine, of the class of 4-substituted-2,5-dimethoxyamphetamines or “DOx” compounds. Without being bound by theory, DOx compounds generally have methoxy groups at the 2- and 5-positions of the phenyl ring, and a substituent such as alkyl or halogen at the 4-position of the phenyl ring. Such compounds may be potent and long-lasting agents that are highly selective 5-HT_{2A}, 5-HT_{2B}, and/or 5-HT_{2C} receptor partial agonists. Non-limiting examples of “DOx compounds” useful in the practice of the invention include 2,5-Dimethoxy-4-amylamphetamine (or, for shorthand, “2,5-Dimethoxy-4-amylA,” where “A” stands for “amphetamine”; DOAM), 2,5-Dimethoxy-4-bromoA (DOB), 2,5-Dimethoxy-4-butylA (DOBU), 2,5-Dimethoxy-4-chloroA (DOC), 2,5-Dimethoxy-4-ethoxyA (MEM), 2,5-Dimethoxy-4-(methoxymethyl)A (DOMOM), 2,5-Dimethoxy-4-(ethoxymethyl) A (DOMOE), 2,5-Dimethoxy-4-ethylA (DOET), 2,5-Dimethoxy-4-ethyl-thioA (Aleph-2), 2,5-Dimethoxy-4-fluoroA (DOF), 2,5-Dimethoxy-4-(2-fluoroethyl)A (DOEF), 2,5-Dimethoxy-4-(3-fluoropropyl)A (DOPF), 2,5-Dimethoxy-4-iodoA (DOI), 2,5-Dimethoxy-4-isopropylthioA (Aleph-4), 2,5-Dimethoxy-4-methylA (DOM), 2,5-Dimethoxy-4-methylthioA (Aleph-1), 2,5-Dimethoxy-4-nitroA (DON), 2,5-Dimethoxy-4-phenylthio-A (Aleph-6), 2,5-Dimethoxy-4-benzylA (DOBZ), 2,5-Dimethoxy-4-(3-methoxybenzyl)A (DO3MeOBZ), 2,5-Dimethoxy-4-[(tetrahydrofuran-2-yl)methyl]A (DOTHFM), 2,5-Dimethoxy-4-propylA (DOPR), 2,5-Dimethoxy-4-isopropylA (DOiP), 2,5-Dimethoxy-4-propylthioA (Aleph-7), 2,5-Dimethoxy-4-(difluoromethyl)A (DODFM), 2,5-Dimethoxy-4-trifluoromethylA (DOTFM), 2,5-Dimethoxy-4-(2,2,2-trifluoroethyl)A (DOTFE), 2,5-Dimethoxy-4-cyanoA (DOCN), 2,5-Dimethoxy-4-ethynyl-A (DOYN), Dimoxamine (“Ariadne”) (4C-D), 1-(2,5-Dimethoxy-4-ethyl-phenyl) butan-2-amine (4C-E), 1-(2,5-Dimethoxy-4-(n-propyl)phenyl)butan-2-amine (4C-P), 1-(2,5-Dimethoxy-4-bromophenyl)butan-2-amine (4C-B), 1-(2,5-Dimethoxy-4-chloro-phenyl)butan-2-amine (4C-C), 1-(2,5-Dimethoxy-4-iodophenyl)butan-2-amine (4C-I), 1-(2,5-Dimethoxy-4-nitrophenyl)butan-2-amine (4C-N), 1-[2,5-Dimethoxy-4-(ethylthio)phenyl]butan-2-amine (4C-T-2), DimethoxymethA (“Beatrice”) (N-methyl-DOM), 2,5-Dimethoxy-3,4-methylenedioxyA (DMMDA), 2,5-dimethoxy-3,4-dimethylA (“Ganesha”) (3-methyl-DOM), 2,5-Dimethoxy-3,4-trimethylenylA (G-3), 2,5-Dimethoxy-3,4-tetramethylenylA (G-4), 2,5-Dimethoxy-3,4-norbornylA (G-5), 1-(5,8-dimethoxy-3,4-dihydro-1H-isochromen-7-yl)propan-2-amine (G-0), 2,5-Dimethoxy-3,4-dichloroA (DODO), IDNNA (IDNNA), Methyl-DOB (_{2A}N-methyl-DOB), 2,3,4,5-TetramethoxyA (2,3,4,5-TetramethoxyA), 1-(4-Bromo-2,3,6,7-tetrahydrofuro [2,3-f][1]benzofuran-8-yl)propan-2-amine (DOB-FLY), and Bromo-DragonFLY (DOB-DFLY).

In some embodiments, a phenethylamine will be a substituted phenethylamine having the structure below, wherein R^{N}, R^{α}, R^{β}, and R²-R⁶ will be as taught herein and as generally understood in the art:

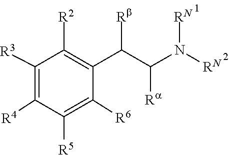

For example, in some embodiments, R^{N}, R^{α}, R^{β}, and R²⁻⁶ are independently hydrogen, deuterium, halogen, C₁-C₅ alkyl, C₂-C₈ alkenyl, C₂-C₈ alkynyl, C₃-C₈ cycloalkyl (independently or ring closed with the nitrogen, when R^{N}), C₃-C₈ cycloalkenyl (independently or ring closed with the nitrogen, when R^{N}), aryl, or heterocyclyl; including where R³ and R⁴ may be joined together to form a dioxole (as with MDMA), a furan, a tetrahydrofuran, a thiophene, a pyrrole, a pyridine, a pyrrolidine, an ethylene oxide, an ethylenimine, a trimethylene oxide, a pyran, a piperidine, an imidazole, a thiazole, a dioxane, a morpholine, a pyrimidine, or otherwise so as to create a benzene heterocycle; and any of which are optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate, —OP(O)(OH)₂, —OC(O)H, —OSO₂OH, —OC(O)NH₂, and —SONH. In some embodiments, a psychedelic phenethylamine is a tertiary amine wherein the additional R^{N} is independently defined as above, and with all other substituents as above. In addition, phenethylamines useful in the practice of the invention also necessarily include any salts, isomers (including structural isomers and stereoisomers, such as enantiomers), precursors, prodrugs, metabolites, derivatives, analogs, isotopologs, or variants thereof, or a combination thereof.

Other phenethylamines and tryptamines useful in disclosed embodiments will be as known in the art (see, e.g., Grob & Grigsby 2021, Luethi & Liechti 2020, Nichols 2016, Glennon 1999). For example, more than 300 “new psychoactive substances” (NPSs) have been synthesized in increasing numbers since the beginning of the twenty-first century. These substances belong to multiple chemical classes such as tryptamines, phenethylamines, cathinones, cannabinoids, and piperazines (EMCDDA, 2014). Whereas almost all contemporary research is accounted for by a small handful of compounds, and in particular the “classic” psychedelics, such NPSs (e.g., Winter, 2009) also may have clinical utility when used in the combinations, compositions, and methods of the invention, according to this disclosure.

a. Exemplary 5-HT₉ Agents Useful in Embodiments of the Invention

In some exemplary embodiments, the 5-HT₂ agent is any of 3-[2-(Dimethylamino)ethyl]-1-methylindol-4-ol (1-methylpsilocin), 1-(5-methoxy-1H-indol-3-yl) propan-2-amine (5-MeO-αMTb), 2-(4-bromo-2,3,6,7-tetra-hydrofuro[2,3-f][1]benzofuran-8-yl)ethanamine (2C-B-FLY), (S)-(+)-1-(2-Aminopropyl)-8,9-dihydropyrano[3,2-e]indole (AL-37350A), 2-[(3-Chlorophenyl)methoxy]-6-(1-piperazinyl)pyrazine (CP 809101), N-Allyl-N-[2-(1H-indol-3-yl)ethyl]prop-2-en-1-amine (DALT), 4-bromo-2,5-dimethoxyamphetamine (DOB), 2,5-dimethoxy-4-ethylamphetamine (DOET), 1-(4-hexyl-2,5-dimethoxy-phenyl)propan-2-amine (DOHx), 2,5-dimethoxy-4-ethoxyamphetamine (MEM), 6-Chloro-2-(1-piperazinyl) pyrazine (CPP; MK-212), 4-[2-[[(2-hydroxyphenyl)methyl]amino]ethyl]-2,5-dimethoxy benzonitrile (NBOH-2C-CN), (4-Bromo-3,6-dimethoxybenzocyclobuten-1-yl)methylamine (TCB-2), 8,9-Dichloro-2,3,4,4a-tetrahydro-1H-pyrazino[1,2-a]quinoxalin-5(6H)-one (WAY 161503), and any pharmaceutically acceptable salt (e.g., hydrochloride, hydrobromide, fumarate), hydrate, solvate, precursor, prodrug, metabolite, derivative, analog, isotopolog, variant, tautomer, isomer, or stereoisomer (including pure or substantially pure individual enantiomers and enantiomerically enriched mixtures having any enantiomeric excess greater than 0%) thereof, or a combination thereof (including all amorphous and polymorphic forms).

One of skill will appreciate the ability to determine which other compounds are useful agents in the practice of disclosed embodiments, by reference to the teachings herein in combination with the general knowledge in the art, such as by reference to DrugCentral, DrugBank, and other compendia of drug data.

### E. Serotonin 7 (5-HT₇) Agents

In some embodiments a therapeutic combination will comprise a serotonin-7 agent. In some embodiments, a “serotonin-7 agent” may refer to a compound that binds to, blocks, activates, inhibits, or otherwise influences (e.g., via an allosteric reaction) activity at a serotonin-7 (5-HT₇) receptor. As used herein, the terms “serotonin-7 receptor” and “5-HT₇ receptor” refer to any of the 5-HT₇ receptor subtypes (i.e., HT₇₍ₐ₎, HT_{7(b)}, HT_{7(d)}), or the 5-HT₇ receptor system as a whole. In some embodiments, the 5-HT₇ agent acts as a receptor modulator, wherein the 5-HT₇ agent alters the function of a receptor to which it binds, the alteration including any of activating the activity of the receptor, inhibiting the activity of the receptor, and activating or inhibiting the activity of the receptor, depending on the dose administered.

Many compounds that possess affinity for one serotonin receptor subfamily can also bind to serotonin receptors of other subfamilies, or possess affinity for receptors of the serotonin receptor system as a whole. Accordingly, in some embodiments, the 5-HT₇ agent is a compound that also has affinity for another serotonin receptor. In embodiments, the 5-HT₇ agent is a phenethylamine or tryptamine disclosed herein as a serotonin receptor agent, which may also have affinity for other serotonin receptor subfamilies in addition to the 5-HT₇ receptor. However, 5-HT₇ agents can also be differentiated from other serotonin receptor agents, for example according to potency at the 5-HT₇ receptor (e.g., measured in absolute terms, such as Kᵢ), or by their selectivity (e.g., as determined by comparing the Kᵢ values to calculate selectivity over a given receptor) for the 5-HT₇ receptor subfamily, or a specific 5-HT₇ receptor subtype, relative to another receptor system, or relative to another serotonin receptor subfamily (e.g., 5-HT₂).

In some embodiments, the 5-HT₇ agent has a 5-HT₇ receptor Kᵢ of less than 5 nM, less than 10 nM, less than 15 nM, less than 20 nM, less than 25 nM, less than 50 nM, less than 100 nM, or less than 1 μM. In some embodiments, the 5-HT₇ agent has a 5-HT₇ receptor Kᵢ of less than 5 nM. In some embodiments, the 5-HT₇ agent has a 5-HT₇ receptor Kᵢ of less than 10 nM. In some embodiments, the 5-HT₇ agent has a 5-HT₇ receptor Kᵢ of less than 15 nM. In some embodiments, the 5-HT₇ agent has a 5-HT₇ receptor Kᵢ of less than 20 nM. In some embodiments, the 5-HT₇ agent has a 5-HT₇ receptor Kᵢ of less than 25 nM. In some embodiments, the 5-HT₇ agent has a 5-HT₇ receptor Kᵢ of less than 50 nM. In some embodiments, the 5-HT₇ agent has a 5-HT₇ receptor Kᵢ of less than 100 nM. In some embodiments, the 5-HT₇ agent has a 5-HT₇ receptor Kᵢ of less than 1 μM. In some embodiments, the 5-HT₇ agent has a 5-HT₇ receptor Kᵢ of between about 1 and 5 nM, 5 and 10 nM, 5 and 15 nM, 5 and 20 nM, 5 and 25 nM, 5 and 50 nM, 5 and 100 nM, and 5 nM and 1 μM. In some embodiments, the 5-HT₇ agent has a 5-HT₇ receptor Kᵢ of between about 1 and 5 nM. In some embodiments, the 5-HT₇ agent has a 5-HT₇ receptor Kᵢ of between about 5 and 10 nM. In some embodiments, the 5-HT₇ agent has a 5-HT₇ receptor Kᵢ of between about 5 and 15 nM. In some embodiments, the 5-HT₇ agent has a 5-HT₇ receptor Kᵢ of between about 5 and 20 nM. In some embodiments, the 5-HT₇ agent has a 5-HT₇ receptor Kᵢ of between about 5 and 25 nM. In some embodiments, the 5-HT₇ agent has a 5-HT₇ receptor Kᵢ of between about 5 and 50 nM. In some embodiments, the 5-HT₇ agent has a 5-HT₇ receptor Kᵢ of between about 5 and 100 nM. In some embodiments, the 5-HT₇ agent has a 5-HT₇ receptor Kᵢ of between about 5 nM and 1 μM.

In some embodiments, the 5-HT₇ agent is a “selective agent,” which may refer to an agent that binds to the 5-HT₇ receptor system more selectively than receptors not part of the 5-HT₇ receptor system. In some embodiments, such selectivity (also termed “specificity” herein), is expressed as a ratio greater than 1.0. In some embodiments, selectivity (as measured via Kᵢ) for the 5-HT₇ receptor system is greater than 1.0, such as but not limited to about 1.1:1, 1.5:1, 2:1, 5:1, 10:1, 15:1, 25:1, 50:1, 100:1, etc., as compared to receptors not part of the 5-HT₇ receptor system. In embodiments, selectivity (as Kᵢ) for the 5-HT₇ receptor system is greater than 1.0, such as but not limited to about 1.1:1, 1.5:1, 2:1, 5:1, 10:1, 15:1, 25:1, 50:1, 100:1, etc., as compared to 5-HT₂ receptors, such as 5-HT_{2A}, 5-HT_{2B}, and/or 5-HT_{2C} receptors. In embodiments, selectivity (as Kᵢ) for the 5-HT₇ receptor system is greater than 1.0, such as but not limited to about 1.1:1, 1.5:1, 2:1, 5:1, 10:1, 15:1, 25:1, 50:1, 100:1, etc., as compared to 5-HT₁ receptors. In embodiments, selectivity (as Kᵢ) for the 5-HT₇ receptor system is greater than 1.0, such as but not limited to about 1.1:1, 1.5:1, 2:1, 5:1, 10:1, 15:1, 25:1, 50:1, 100:1, etc., as compared to 5-HT₂ receptors. In some embodiments, such selectivity (also termed “specificity” herein), is expressed as a ratio less than 1.0. In some embodiments, selectivity (as Kᵢ) for the 5-HT₇ receptor system is less than 1.0, such as but not limited to about 0.9:1, 0.8:1, 0.7:1, 0.6:1, 0.5:1, 0.4:1, 0.3:1, 0.2:1, 0.1:1, etc., as compared to receptors not part of the 5-HT₇ receptor system.

In some embodiments, the selectivity (as Kᵢ) for the 5-HT₇ receptor system is between about 0.5:1 and 500:1, as compared to receptors not part of the 5-HT₇ receptor system. In some embodiments, the selectivity (as Kᵢ) for the 5-HT₇ receptor system is between about 0.5:1 and 1:1, as compared to receptors not part of the 5-HT₇ receptor system. In some embodiments, the selectivity (as Kᵢ) for the 5-HT₇ receptor system is about 0.6:1, as compared to receptors not part of the 5-HT₇ receptor system. In some embodiments, the selectivity (as Kᵢ) for the 5-HT₇ receptor system is between about 50:1 and 500:1, as compared to receptors not part of the 5-HT₇ receptor system. In some embodiments, the selectivity (as Kᵢ) for the 5-HT₇ receptor system is between about 100:1 and 500:1, as compared to receptors not part of the 5-HT₇ receptor system. In some embodiments, the selectivity (as Kᵢ) for the 5-HT₇ receptor system is between about 100:1 and 300:1, as compared to receptors not part of the 5-HT₇ receptor system. In some embodiments, the selectivity (as Kᵢ) for the 5-HT₇ receptor system is between about 150:1 and 250:1, as compared to receptors not part of the 5-HT₇ receptor system. In embodiments, the selectivity (as Kᵢ) for the 5-HT₇ receptor system is about 200:1, as compared to receptors not part of the 5-HT₇ receptor system.

In some embodiments, the selectivity (as Kᵢ) for the 5-HT₇ receptor system is between about 0.5:1 and 500:1, as compared to 5-HT₂ receptors. In some embodiments, the selectivity (as Kᵢ) for the 5-HT₇ receptor system is between about 0.5:1 and 1:1, as compared to 5-HT₂ receptors. In some embodiments, the selectivity (as Kᵢ) for the 5-HT₇ receptor system is about 0.6:1, as compared to 5-HT₂ receptors. In some embodiments, the selectivity (as Kᵢ) for the 5-HT₇ receptor system is between about 50:1 and 500:1, as compared to 5-HT₂ receptors. In some embodiments, the selectivity (as Kᵢ) for the 5-HT₇ receptor system is between about 100:1 and 500:1, as compared to 5-HT₂ receptors. In some embodiments, the selectivity (as Kᵢ) for the 5-HT₇ receptor system is between about 100:1 and 300:1, as compared to 5-HT₂ receptors. In some embodiments, the selectivity (as Kᵢ) for the 5-HT₇ receptor system is between about 150:1 and 250:1, as compared to 5-HT₂ receptors. In some embodiments, the selectivity (as Kᵢ) for the 5-HT₇ receptor system is about 200:1, as compared to 5-HT₂ receptors.

In some embodiments, a serotonin-7 agent is an agonist. Broadly, a “serotonin-7 receptor agonist” (or a “serotonin-7 agonist,” “5-HT₇ receptor agonist,” or “5-HT₇ agonist”) is a compound that initiates a biological response when bound to a serotonin-7 receptor. In some embodiments, the 5-HT₇ agonist has a high specificity for 5-HT₇ receptors. In some embodiments, the 5-HT₇ agonist has a low specificity for receptors not part of the serotonin-7 receptor system.

In some embodiments, a serotonin-7 agent is an antagonist. Broadly, a “serotonin-7 receptor antagonist” (or a “serotonin-7 antagonist,” “5-HT₇ receptor antagonist,” or “5-HT₇ antagonist”) is a compound that inhibits a biological response when bound to a serotonin-7 receptor. In some embodiments, the 5-HT₇ antagonist has a high specificity for 5-HT₇ receptors. In some embodiments, the 5-HT₇ antagonist has a low specificity for receptors not part of the serotonin-7 receptor system.

In some embodiments, a serotonin-7 agent is an inverse agonist. Broadly, a “serotonin-7 receptor inverse agonist” (or a “serotonin-7 inverse agonist,” “5-HT₇ receptor inverse agonist,” or “5-HT₇ inverse agonist” is a compound that, when bound to a serotonin-7 receptor, lowers the serotonin-7 receptor's constitutive activity to a level below its basal state. In some embodiments, the 5-HT₇ inverse agonist has a high specificity for 5-HT₇ receptors. In some embodiments, the 5-HT₇ inverse agonist has a low specificity for receptors not part of the serotonin-7 receptor system.

Exemplary 5-HT₇ agents include, but are not limited to, tryptamines (including ergolines and other complex tryptamines), phenylalkylamines, and phenethylamines, such as, but not limited to, those disclosed herein, including such as described above for 5-HT_{2A}. In certain embodiments, it will be understood that tryptamines and including such complex tryptamines as ergolines (generally, and broadly as chemical classes) will have greater relative affinity for 5-HT₇ than phenethylamines and other phenylalkylamines (generally, and broadly as chemical classes), and that such determinations will be readily made by those of ordinary skill in view of the teachings herein and the general knowledge in the art (see, e.g., Ray 2010).

In some embodiments, exemplary 5-HT₇ agents will include (and in some embodiments, in descending rank order of preference, based on descending relative affinity at 5-HT₇), the tryptamines DMT, 6-F-DMT, 5-MeO-MiPT, LSD, 5-MeO-DMT, 5-MeO-TMT, cis-2a, DPT, 5-MeO-DiPT, psilocin, RR-2b, EMDT, lisuride, DiPT, SS-2c; and the phenylalkylamines TMA, 2C-B, 2C-E, and MDA (see also, e.g., Ray 2010).

a. Exemplary 5-HT₇ Agents Useful in Embodiments of the Invention

In some embodiments, the 5-HT₇ agent is 5-Carboxamidotryptamine (5-CT), N-[2-(5-methoxy-1H-indol-3-yl)ethyl]-N-(prop-2-en-1-yl)prop-2-en-1-amine (5-MeO-DALT), 5-methoxy-N,N-dimethyl-tryptamine (5-MeO-DMT); other exemplary 5-HT₇ agents include N-isopropyl-5-methoxy-N-methyltryptamine (5-MeO-MiPT) (“Moxy”), 3-(3-ethylimidazol-4-yl)-5-iodo-1H-indole (AGH-107), 3-(3-ethylimidazol-4-yl)-4-fluoro-5-iodo-1H-indole (AGH-192), 1-(1H-Indol-3-yl)propan-2-amine (AMT), 7-{4-[4-(2,3-Dichlorophenyl) piperazin-1-yl]butoxy}-3,4-dihydroquinolin-2(1H)-one (aripiprazole), (2S)—N,N-dimethyl-5-(1,3,5-trimethyl-pyrazol-4-yl)-1,2,3,4-tetrahydronaphthalen-2-amine, (AS-19), N, N-dimethyl-3-(1,3,5-trimethyl-1H-pyrazol-4-yl)phenethylamine (E-55888), N-(4-cyanophenylmethyl)-4-(2-diphenyl)-1-piperazinehexanamide (LP-211), (4-[2-(Methylthio)phenyl]-N-(1,2,3,4-tetrahydro-1-naphthalenyl)-1-piperazinehexanamide) (LP-44), (6aR,9R)—N,N-diethyl-7-methyl-4,6,6a,7,8,9-hexahydroindolo[4,3-fg]quinoline-9-carboxamide (LSD), or a pharmaceutically acceptable salt, hydrate, solvate, precursor, prodrug, metabolite, derivative, analog, isotopolog, variant, tautomer, isomer, or stereoisomer (including pure or substantially pure individual enantiomers and enantiomerically enriched mixtures having any enantiomeric excess greater than 0%) thereof, or a combination thereof (and including all amorphous and polymorphic forms).

In embodiments, the 5-HT₇ agent is DMT, or a pharmaceutically acceptable salt thereof. In embodiments, the 5-HT₇ agent is 6-F-DMT, or a pharmaceutically acceptable salt thereof. In embodiments, the 5-HT₇ agent is 5-MeO-MiPT, or a pharmaceutically acceptable salt thereof. In embodiments, the 5-HT₇ agent is LSD, or a pharmaceutically acceptable salt thereof. In embodiments, the 5-HT₇ agent is 5-MeO-DMT, or a pharmaceutically acceptable salt thereof. In embodiments, the 5-HT₇ agent is 5-MeO-TMT, or a pharmaceutically acceptable salt thereof. In embodiments, the 5-HT₇ agent is cis-2a, or a pharmaceutically acceptable salt thereof. In embodiments, the 5-HT₇ agent is 5-MeO-DiPT, or a pharmaceutically acceptable salt thereof. In embodiments, the 5-HT₇ agent is psilocin, or a pharmaceutically acceptable salt thereof. In embodiments, the 5-HT₇ agent is RR-2b, or a pharmaceutically acceptable salt thereof. In embodiments, the 5-HT₇ agent is EMDT, or a pharmaceutically acceptable salt thereof. In embodiments, the 5-HT₇ agent is lisuride, or a pharmaceutically acceptable salt thereof. In embodiments, the 5-HT₇ agent is DiPT, or a pharmaceutically acceptable salt thereof. In embodiments, the 5-HT₇ agent is SS-2c, or a pharmaceutically acceptable salt thereof. In embodiments, the 5-HT₇ agent is TMA, or a pharmaceutically acceptable salt thereof. In embodiments, the 5-HT₇ agent is 2C-B, or a pharmaceutically acceptable salt thereof. In embodiments, the 5-HT₇ agent is 2C-E, or a pharmaceutically acceptable salt thereof. In embodiments, the 5-HT₇ agent is MDA, or a pharmaceutically acceptable salt thereof.

In embodiments, including some preferred embodiments, the 5-HT₇ agent is 5-MeO-DMT or 5-MeO-MiPT, or a pharmaceutically acceptable salt thereof.

One of skill will appreciate the ability to determine which other compounds are useful agents in the practice of disclosed embodiments, by reference to the teachings herein in combination with the general knowledge in the art, such as by reference to DrugCentral, DrugBank, and other compendia of drug data.

### F. Receptor Selectivity and Modulatory Activity

Provided are combinations of agents that display selectivity for receptors that contribute to an entactogenic mindstate. In embodiments, imidazoline-1 (I₁), 5-HT₂, 5-HT₇, and CB₁ are among the receptors that contribute to the elicitation of an entactogenic mindstate. In embodiments, an agent provided herein is selective for one or more of I₁, 5-HT₂, 5-HT₇, and CB₁ receptors. In embodiments, the combinations provided herein display selectivity for I₁ and one or more of 5-HT₂, 5-HT₇, and CB₁ receptors. In embodiments, an agent provided herein modulates activity at one or more of I₁, 5-HT₂, 5-HT₇, and CB₁ receptors. In embodiments, the combinations provided herein modulate activity at I₁ and one or more of 5-HT₂, 5-HT₇, and CB₁ receptors. In embodiments, an agent provided herein selectively modulates activity at one or more of I₁, 5-HT₂, 5-HT₇, and CB₁ receptors. In embodiments, the combinations provided herein selectively modulate activity at I₁ and one or more of 5-HT₂, 5-HT₇, and CB₁ receptors. In some embodiments, the combinations provided herein are therapeutic combinations that elicit an entactogenic mindstate.

a. Selectivity

The therapeutic combinations provided herein may comprise one or more agents that are selective for one or more targets. Selectivity of an agent for a target, such as a receptor, can be defined in different contexts. In some embodiments, an agent is selective for a receptor if it displays its highest relative affinity for said receptor. In some embodiments, an agent is selective for a receptor if it acts exclusively at said receptor. In some embodiments, a selective agent neither exhibits its greatest affinity nor acts exclusively at a target receptor but instead displays a relatively greater affinity for one receptor over another. Notably, the selectivity of a compound is determined in the context of assayed targets and receptors. In some embodiments, selectivity of a ligand for its target, such as a receptor, can be expressed as a measure of binding strength, referred to as binding affinity. In some embodiments, selectivity is assessed by determining a measure of binding that is available to one of skill in the art, for example, one or more of a dissociation constant (K_{D}), an association constant (K_{A}), and an inhibition constant (Kᵢ). In some embodiments, the target receptor is I₁, 5-HT₂, 5-HT₇, or CB₁. In some embodiments, a provided therapeutic combination comprising agents, such as selective agents, elicits an entactogenic mindstate.

In some embodiments, selectivity can be evaluated by determining binding affinity, such as that of a ligand, for example, an agent described herein, and a target, for example, a receptor described herein. Methods of determining binding affinity are known to those of skill in the art and include radioligand binding assays (Maguire, 2012; Roth, 2018). Radioligand binding experiments represent sensitive and quantitative techniques for determining affinity, such as binding affinity of a compound and a receptor.

In some embodiments, selectivity can be evaluated by determining the inhibition constant of an agent provided herein and a receptor system, for example, I₁, 5-HT₂, 5-HT₇, or CB₁. An inhibition constant (Kᵢ) may be described as the concentration at which 50% of a radiolabeled ligand, such as an agonist, is displaced by the test ligand. Accordingly, as presented in the Cheng-Prusoff equation, an observed IC₅₀ can be used to determine Kᵢ, where Ki=IC50/(1+[R]/Kd), where the IC₅₀ is the concentration of the competitive inhibitor producing a 50% inhibition, R is the concentration of radioligand used in the competition binding assay, and Kd is the equilibrium dissociation constant of the radioligand in the assay.

A competition binding assay or radioligand displacement assay also can be used to determine Kᵢ according to methods known in the art (Toro-Sazo, 2019). In some examples, displaced radioligands may be antagonists, e.g., [³H]Ketanserin for 5-HT_{2A} and [³H]mesulergine for 5-HT_{2C}. In some examples, displaced radioligands may be agonists, for example [³H]LSD for 5-HT_{2B}.

In another example of determining Kᵢ, one or more agents are first screened against various receptor systems to determine activity at a given receptor prior to determining Kᵢ. As described by Glennon et al., a compound is initially assayed at 10 μM against each receptor, transporter or ion channel. Those that induced >50% inhibition are then assayed at 1, 10, 100, 1,000, and 10,000 nM to determine Kᵢ values. Each Kᵢ value is then calculated from at least three replicated assays (Glennon, 2000).

In some embodiments, such measures described herein, and others known to one of skill in the art, are compared to determine relative affinity. In some embodiments, measures of binding affinity, such as K_{A} or K_{D}, are compared to determine relative affinity. In some examples, a relatively lower K_{A} indicates weaker binding affinity, and a higher K_{A} indicates greater binding affinity. In some examples, a relatively higher K_{D} indicates weaker binding affinity, and a lower K_{D} indicates stronger binding affinity. In some embodiments, equilibrium inhibition constants (Kᵢ) are compared to determine relative affinity. In some examples, a relatively higher Kᵢ indicates weaker affinity, and a lower Kᵢ indicates stronger affinity.

In some examples, Kᵢ values distributed over several orders of magnitude may be log-transformed to facilitate comparison, as described by Ray et al. For example, by calculating pKi=−log₁₀(Ki). In such examples, higher pKᵢ values are indicative of higher affinities. In some examples, the log-transformed values may then be normalized. Normalizing such values provides advantages for ease of comparison, including factoring out potency so that agents of different potencies can be directly compared. In one example, values were normalized by adjusting the highest pKi value for each drug to a value of 4, and all Kᵢ values reported as >10,000 were set to a value of zero (Ray, 2010). In some embodiments, relative affinity may be determined by comparing transformed and/or normalized measures described herein. Only values that have been subjected to the same transformation(s) will be compared in determining relative affinity.

In embodiments, selective affinity can be measured by known methods that include using the “NIMH Psychoactive Drug Screening Program (PDSP)” Kᵢ database (available, at the time of filing, at https://pdsp.unc.edu/databases/kidb.php), which is a public domain resource that provides information on the abilities of drugs to interact with different molecular targets. The PDSP Kᵢ database serves as a data warehouse for published and internally-derived pKᵢ, or affinity, values for a large number of drugs and drug candidates at an expanding number of G-protein coupled receptors, ion channels, transporters and enzymes. The PDSP internet site also provides for commonly used protocols and assays for measuring pKᵢ values, for example at different 5-HT receptors, and such other receptor systems discussed herein.

In some embodiments, the affinity of an agent for a receptor is compared to the affinity of the same agent for a different receptor to determine relative affinity. In some embodiments, an agent provided herein displays a relative affinity for one or more targeted receptors that is at least a factor of 1.1, such as at least a factor of 2, at least a factor of 3, at least a factor of 4, at least a factor 5, at least a factor of 6, at least a factor of 7, at least a factor of 8, at least a factor of 9, at least a factor of 10, at least a factor of 11, at least a factor of 12, at least a factor of 20, at least a factor of 30, at least a factor of 50, at least a factor of 100, at least a factor of 200, at least a factor of 500, and values in between, when compared to non-targeted receptors. In some embodiments, the targeted receptor is an I₁, 5-HT₂, 5-HT₇, or a CB₁ receptor. In some embodiments, an agent that displays relatively high selectivity for an I₁, 5-HT₂, 5-HT₇, or a CB₁ receptor is selected for use in a therapeutic combination described herein.

In embodiments, the affinity of an agent for a receptor is compared to the affinity of a different agent for the same receptor to determine relative affinity. In embodiments, an agent provided herein displays a relative affinity for one or more targeted receptors that is at least a factor of 1.1, such as at least a factor of 2, at least a factor of 3, at least a factor of 4, at least a factor 5, at least a factor of 6, at least a factor of 7, at least a factor of 8, at least a factor of 9, at least a factor of 10, at least a factor of 11, at least a factor of 12, at least a factor of 20, at least a factor of 30, at least a factor of 50, at least a factor of 100, at least a factor of 200, at least a factor of 500, and values in between, when compared to the affinity of a different agent for the same receptor. In embodiments, the targeted receptor is an I₁, 5-HT₂, 5-HT₇, or CB₁ receptor (i.e., a receptor of the I₁, 5-HT₂, 5-HT₇, or CB₁ receptor systems). In embodiments, an agent that displays relatively high selectivity for an I₁, 5-HT₂, 5-HT₇, or CB₁ receptor (i.e., a selective agent for any of the I₁, 5-HT₂, 5-HT₇, or CB₁ receptor systems) is selected for use in a therapeutic combination described herein.

In some embodiments, a disclosed entactogenic combination comprises agents that are selective, or relatively highly selective, for one or more of I₁, 5-HT₂, 5-HT₇, and CB₁ receptors (i.e., one or more of the I₁, 5-HT₂, 5-HT₇, and CB₁ receptor systems). In some embodiments, an agent that is selective for one or more receptors (or, as similarly stated, and as used elsewhere interchangeably, unless context indicates otherwise, “receptor systems”) modulates the activity at said receptors (i.e., receptor systems). In some embodiments, a therapeutic combination comprising selective agents modulates the activity at said receptors, for example, I₁ and one or more of 5-HT2, 5-HT7, and CB₁ receptors. In some embodiments, the affinity measure may be determined by a binding assay, such as a radioligand binding assay, or a binding assay that incorporates functional activity, such as by measuring the functional response of a system to a drug in the present of an antagonist, as is known to one of skill in the art.

### b. Modulatory Activity

Provided are combinations of agents that modulate certain receptors to produce a therapeutic effect, such as an entactogenic mindstate. In some embodiments, I₁, 5-HT₂, 5-HT₇, and CB₁ are among the receptors modulated to provide an entactogenic mindstate. Also provided are selectivity and efficacy profiles for the agents described herein.

In some embodiments, an agent in a disclosed entactogenic combination modulates the activity of the imidazoline-1 (I₁) receptor (I₁ receptor system). In some embodiments, an agent in a disclosed entactogenic combination modulates the activity of the cannabinoid-1 (CB₁) receptor (CB₁ receptor system). In some embodiments, an agent in a disclosed entactogenic combination modulates the activity of the serotonin-2 (5-HT₂) receptor and subtypes thereof (5-HT₂ receptor system). In embodiments, an agent in a therapeutic combination modulates the activity of the serotonin-7 (5-HT₇) receptor (5-HT₇ receptor system).

In some embodiments, agents in a disclosed entactogenic combination modulate the activity of 5-HT₂ and 5-HT₇ receptors. In some embodiments, agents in a disclosed entactogenic combination modulate the activity of I₁ and CB₁ receptors. In some embodiments, agents in a disclosed entactogenic combination modulate the activity of I₁ and 5-HT₂ receptors. In some embodiments, agents in a disclosed entactogenic combination modulate the activity of I₁ and 5-HT₇ receptors. In some embodiments, agents in a disclosed entactogenic combination modulate the activity of I₁ and 5-HT₂, and 5-HT₇ receptors. In some embodiments, agents in a disclosed entactogenic combination modulate the activity of CB₁ and 5-HT₂ receptors. In some embodiments, agents in a disclosed entactogenic combination modulate the activity of CB₁ and 5-HT₇ receptors. In some embodiments, agents in a disclosed entactogenic combination modulate the activity of CB₁, 5-HT₂, and 5-HT₇ receptors. In some embodiments, agents in a disclosed entactogenic combination modulate the activity of I₁, CB₁, and 5-HT₂ receptors. In some embodiments, agents in a disclosed entactogenic combination modulate the activity of I₁, CB₁, and 5-HT₇ receptors. In embodiments, agents in a therapeutic combination modulate the activity of I₁, CB₁, 5-HT₂, and 5-HT₇ receptors.

i. Agonism

In some embodiments, modulation comprises agonistic activity. In embodiments, modulation comprises partial agonism. In embodiments, modulation comprises inverse agonism. In embodiments, agonism is determined using in vitro techniques such as ligand-binding assays, the receptor inactivation method of Furchgott, which includes analyses of the concentration/response curves for agonists before and after partial inactivation of receptors with an irreversible antagonist; or the comparative method in functional assays, which compares concentration-response curves for a full and partial agonist (Strange, 2008).

In some embodiments, agonists are measured by their “efficacy.” Herein, “efficacy,” may refer to a substance's ability to exert effects on a receptor and its associated signalling systems. Imidazoline-1 receptors may contribute to the regulation of sympathetic nervous activity. In one example, I₁-receptor agonist moxonidine activates I₁ receptors in the rostroventrolateral medulla (RVLM), which reduces the activity of the sympathetic nervous system (Ziegler, 1996). In some examples, activity at imidazoline-1 can be determined from electrophysiological recordings of brain cells (Hayar and Guyenet, 2000). Efficacy of the imidazoline-1 agonist monoxidine has been described in norepinephrine release assays (Schafer, 2002). Such measures of efficacy are merely exemplary and should not be construed as limiting.

In some embodiments, receptor activation and efficacy can be determined by evaluating the interactions of components of a GPCR (Harrison, 2003). In an example, 5-HT₂ receptor activation and GPCR subunit interactions are assessed using Bioluminescence Resonance Energy Transfer (BRET) and compared with serotonin as the benchmark (WO2021179091, citing McCorvey, 2020). In another example, CB₁-induced [(35)S]GTPγS binding is used to determine the efficacy of different compounds (DeLuca, 2015). Such measures of efficacy are exemplary and should not be construed as limiting.

In embodiments, measures of efficacy are expressed as, e.g., EC₅₀, as the concentration of an agent that induces a response halfway between baseline and a maximum threshold, Eₘₐₓ, as the maximal effect of the agonist; K_{obs}/EC₅₀, as the ratio of agonist dissociation constant to concentration of agonist giving half maximal effect in functional assay (with K_{obs} referring to measurements of agonist affinity using liganding binding); and EₘₐₓK_{obs}/EC₅₀, as a combined parameter (Strange, 2008). In embodiments, efficacy is assessed by determining the activity in a suitable assay system linked to the receptor using a range of concentrations of the substance under test, yielding a concentration/response experiment (Strange, 2008).

In some embodiments, an agonist exhibits maximal efficacy greater than 0%, including at least 2.5%, at least 5%, at least 7.5%, at least 10%, at least 12.5%, at least 15%, at least 17.5%, at least 20%, at least 22.5%, at least 25%, at least 27.5%, at least 30%, at least 32.5%, at least 35%, at least 37.5%, at least 40%, at least 42.5%, at least 45%, at least 47.5%, at least 50.5%, at least 52.5%, at least 55%, at least 57.5%, at least 60%, at least 62.5%, at least 65%, at least 67.5%, at least 70%, at least 72.5%, at least 75%, at least 77.5%, at least 80%, at least 82.5%, at least 85%, at least 87.5%, at least 90%, at least 92.5%, at least 95%, at least 97.5%, at least 98%, at least 98.5%, at least 99%, at least 99.5%, at least 99.9%, 100%, and values in between, when binding to a target receptor system. In some embodiments, the receptor is I₁, CB₁, 5-HT₂, 5-HT₇, or combinations thereof.

In some embodiments, imidazoline-1 agonists, cannabinoid-1 agonists, serotonin-2 agonists, serotonin-7 agonists, or combinations thereof, refer to compounds having a maximal efficacy greater than 0%, including at least 2.5%, at least 5%, at least 7.5%, at least 10%, at least 12.5%, at least 15%, at least 17.5%, at least 20%, at least 22.5%, at least 25%, at least 27.5%, at least 30%, at least 32.5%, at least 35%, at least 37.5%, at least 40%, at least 42.5%, at least 45%, at least 47.5%, at least 50.5%, at least 52.5%, at least 55%, at least 57.5%, at least 60%, at least 62.5%, at least 65%, at least 67.5%, at least 70%, at least 72.5%, at least 75%, at least 77.5%, at least 80%, at least 82.5%, at least 85%, at least 87.5%, at least 90%, at least 92.5%, at least 95%, at least 97.5%, at least 98%, at least 98.5%, at least 99%, at least 99.5%, at least 99.9%, 100%, and values in between, when binding to the I₁ receptor system, CB₁ receptor system, 5-HT₂ receptor system, 5-HT₇ receptor system, or combinations thereof.

In embodiments, one or more of the provided agents are partial agonists. In some embodiments, any of an I₁ agent, 5-HT₂ agent, 5-HT₇ agent, and/or CB₁ agent may be a partial agonist, i.e., an I₁ partial agonist, 5-HT₂ partial agonist, 5-HT₇ partial agonist, and/or CB₁ partial agonist. Unless context suggests otherwise, an “agonist” will include a “partial agonist,” and therefore unless otherwise specified either explicitly or impliedly, an I₁ agonist includes an I₁ partial agonist, a 5-HT₂ agonist includes a 5-HT₂ partial agonist, a 5-HT₇ agonist includes a 5-HT₇ partial agonist, and a CB₁ agonist includes a CB₁ partial agonist.

In some embodiments, a disclosed agent is a partial agonist, wherein the compound binds to and activates a given receptor with a maximal efficacy of less than 100%, including less than 99.9%, less than 99.5%, less than 99%, less than 97.5%, less than 95%, less than 92.5%, less than 90%, less than 87.5%, less than 85%, less than 82.5%, less than 80%, less than 77.5%, less than 75%, less than 72.5% less than 70%, less than 67.5%, less than 65%, less than 62.5%, less than 65%, less than 62.5%, less than 60%, less than 57.5%, less than 55%, less than 52.5%, less than 50%, less than 47.5%, less than 45%, less than 42.5%, less than 40%, less than 37.5%, less than 35%, less than 32.5%, less than 30%, less than 27.5%, less than 25%, less than 22.5%, less than 20%, less than 17.5%, less than 15%, less than 12.5%, less than 10%, less than 7.5%, less than 5%, less than 2.5%, or any values in between the numbers listed, as compared to an agonist having the capability of reaching 100% efficacy, such as serotonin as a reference agonist for a serotonin receptor or serotonin receptor system.

In some embodiments, a disclosed agent is a partial agonist for imidazoline-1, wherein the compound activates imidazoline-1 with a maximal efficacy of less than 100% at the I₁ receptor.

In some embodiments, a disclosed agent is a partial agonist for cannabinoid-1, wherein the compound activates cannabinoid-1 with a maximal efficacy of less than 100% at the CB₁ receptor.

In some embodiments, a disclosed agent is a partial agonist for serotonin-2, wherein the compound activates serotonin-2 with a maximal efficacy of less than 100% at the 5-HT₂ receptor.

In some embodiments, a disclosed agent is a partial agonist for serotonin-7, wherein the compound activates serotonin-7 with a maximal efficacy of less than 100% at the 5-HT₇ receptor.

In some embodiments, a disclosed agent is a partial agonist for imidazoline-1 and cannabinoid-1, wherein the activates imidazoline-1 and cannabinoid-1 with a maximal efficacy of less than 100% at the I₁ and CB₁ receptors.

In some embodiments, a disclosed agent is a partial agonist for imidazoline-1 and serotonin-2, wherein the compound activates imidazoline-1 and serotonin-2 with a maximal efficacy of less than 100% at the I₁ and 5-HT₂ receptors.

In some embodiments, a disclosed agent is a partial agonist for imidazoline-1 and serotonin-7, wherein the compound activates imidazoline-1 and serotonin-7 with a maximal efficacy of less than 100% at the I₁ and 5-HT₇ receptors.

In some embodiments, a disclosed agent is a partial agonist for cannabinoid-1 and serotonin-2, wherein the compound activates cannabinoid-1 and serotonin-2 with a maximal efficacy of less than 100% at the CB₁ and 5-HT₂ receptors.

In some embodiments, a disclosed agent is a partial agonist for cannabinoid-1 and serotonin-7, wherein the compound activates cannabinoid-1 and serotonin-7 with a maximal efficacy of less than 100% at the CB₁ and 5-HT₇ receptors.

In some embodiments, a disclosed agent is a partial agonist for serotonin-2 and serotonin-7, wherein the compound activates serotonin-2 and serotonin-7 with a maximal efficacy of less than 100% at the 5-HT₂ and 5-HT₇ receptors.

In some embodiments, a disclosed agent is a partial agonist for imidazoline-1, cannabinoid-1, and serotonin-2, wherein the compound binds to and activates imidazoline-1, cannabinoid-1, and serotonin-2 with a maximal efficacy of less than 100% at the I₁, CB₁, and 5-HT₂ receptors.

In some embodiments, a disclosed agent is a partial agonist for imidazoline-1, cannabinoid-1, and serotonin-7, wherein the compound binds to and activates imidazoline-1, cannabinoid-1, and serotonin-7 with a maximal efficacy of less than 100% at the I₁, CB₁, and 5-HT₇ receptors.

In some embodiments, a disclosed agent is a partial agonist for imidazoline-1, serotonin-2, and serotonin-7, wherein the compound binds to and activates imidazoline-1, serotonin-2, and serotonin-7 with a maximal efficacy of less than 100% at the I₁, 5-HT₂, and 5-HT₇ receptors.

In some embodiments, a disclosed agent is a partial agonist for cannabinoid-1, serotonin-2, and serotonin-7, wherein the compound binds to and activates cannabinoid-1, serotonin-2, and serotonin-7 with a maximal efficacy of less than 100% at the CB₁, 5-HT₂, and 5-HT₇ receptors.

In embodiments, a disclosed agent is a partial agonist for imidazoline-1, cannabinoid-1, serotonin-2, and serotonin-7, wherein the compound binds to and activates imidazoline-1, cannabinoid-1, serotonin-2, and serotonin-7 with a maximal efficacy of less than 100% at the I₁, CB₁, 5-HT₂, and 5-HT₇ receptors.

In some embodiments, one or more of the agents provided herein are inverse agonists. In some embodiments, any of an I₁ agent, 5-HT₂ agent, 5-HT₇ agent, and/or CB₁ agent may be an inverse agonist, i.e., an I₁ inverse agonist, 5-HT₂ inverse agonist, 5-HT₇ inverse agonist, and/or CB₁ inverse agonist. Unless context suggests otherwise, an “agonist” will include an “inverse agonist,” and therefore unless otherwise specified either explicitly or impliedly, an I₁ agonist includes an I₁ inverse agonist, a 5-HT₂ agonist includes a 5-HT₂ inverse agonist, a 5-HT₇ agonist includes a 5-HT₇ inverse agonist, and a CB₁ agonist includes a CB₁ inverse agonist.

In some embodiments, a disclosed agent is an inverse agonist for imidazoline-1, wherein the compound binds to the I₁ receptor system and lowers its constitutive activity below that of its basal state.

In some embodiments, a disclosed agent is an inverse agonist for cannabinoid-1, wherein the compound binds to the CB₁ receptor system and lowers its constitutive activity below that of its basal state.

In embodiments, a disclosed agent is an inverse agonist for serotonin-2, wherein the compound binds to the 5-HT₂ receptor system and lowers its constitutive activity below that of its basal state.

In embodiments, a disclosed agent is an inverse agonist for serotonin-7, wherein the compound binds to the 5-HT₇ receptor system and lowers its constitutive activity below that of its basal state.

In some embodiments, a disclosed agent is an inverse agonist for imidazoline-1 and cannabinoid-1, wherein the compound binds to any of the I₁ and CB₁ receptor systems and lowers their constitutive activity below that of its basal state.

In some embodiments, a disclosed agent is an inverse agonist for imidazoline-1 and serotonin-2, wherein the compound binds to any of the I₁ and 5-HT₂ receptor systems and lowers their constitutive activity below that of its basal state.

In some embodiments, a disclosed agent is an inverse agonist for imidazoline-1 and serotonin-7, wherein the compound binds to any of the I₁ and 5-HT₇ receptor systems and lowers their constitutive activity below that of its basal state.

In some embodiments, a disclosed agent is an inverse agonist for cannabinoid-1 and serotonin-2, wherein the compound binds to any of the CB₁ and 5-HT₂ receptor systems and lowers their constitutive activity below that of its basal state.

In some embodiments, a disclosed agent is an inverse agonist for cannabinoid-1 and serotonin-7, wherein the compound binds to any of the CB₁ and 5-HT₇ receptor systems and lowers their constitutive activity below that of its basal state.

In some embodiments, a disclosed agent is an inverse agonist for serotonin-2 and serotonin-7, wherein the compound binds to any of the 5-HT₂ and 5-HT₇ receptor systems and lowers their constitutive activity below that of its basal state.

In some embodiments, a disclosed agent is an inverse agonist for imidazoline-1, cannabinoid-1, and serotonin-2, wherein the compound binds to any of the I₁, CB₁, and 5-HT₂ receptor systems and lowers their constitutive activity below that of its basal state.

In some embodiments, a disclosed agent is an inverse agonist for imidazoline-1, cannabinoid-1, and serotonin-7, wherein the compound binds to any of the I₁, CB₁, and 5-HT₇ receptor systems and lowers their constitutive activity below that of its basal state.

In some embodiments, a disclosed agent is an inverse agonist for imidazoline-1, serotonin-2, and serotonin-7, wherein the compound binds to any of the I₁, 5-HT₂, and 5-HT₇ receptor systems and lowers their constitutive activity below that of its basal state.

In some embodiments, a disclosed agent is an inverse agonist for cannabinoid-1, serotonin-2, and serotonin-7, wherein the compound binds to any of the CB₁, 5-HT₂, and 5-HT₇ receptor systems and lowers their constitutive activity below that of its basal state.

In some embodiments, a disclosed agent is an inverse agonist for imidazoline-1, cannabinoid-1, serotonin-2, and serotonin-7, wherein the compound binds to any of the I₁, CB₁, 5-HT₂, and 5-HT₇ receptor systems and lowers their constitutive activity below that of its basal state.

In some embodiments, a disclosed agent is an inverse agonist, wherein the compound binds to and lowers the constitutive activity of a given receptor below that of its basal state, including lowering its constitutive activity up to 100%, including up to 99.9%, up to 99.5%, up to 99%, up to 97.5%, up to 95%, up to 92.5%, up to 90%, up to 87.5%, up to 85%, up to 82.5%, up to 80%, up to 77.5%, up to 75%, up to 72.5% up to 70%, up to 67.5%, up to 65%, up to 62.5%, up to 65%, up to 62.5%, up to 60%, up to 57.5%, up to 55%, up to 52.5%, up to 50%, up to 47.5%, up to 45%, up to 42.5%, up to 40%, up to 37.5%, up to 35%, up to 32.5%, up to 30%, up to 27.5%, up to 25%, up to 22.5%, up to 20%, up to 17.5%, up to 15%, up to 12.5%, up to 10%, up to 7.5%, up to 5%, up to 2.5%, or any values in between.

In some embodiments, an agonist may be distinguished by its selectivity for a given receptor, or set of receptors. In such embodiments, a compound is an agonist of at least one desired receptor (i.e., any of the I₁, 5-HT₂, 5-HT₇, or CB₁ receptors, or a combination thereof), and possesses a selectivity for the at least one desired receptor that is greater than a ratio of 1.0. Meaning, the ratio of a compound's selectivity (as measured via Kᵢ) for at least one desired receptor system is greater than 1.1:1, 1.5:1, 2:1, 5:1, 10:1, 15:1, 25:1, 50:1, 100:1, etc., as compared to receptors not part of at least one desired receptor system.

ii. Antagonism

In some embodiments, one or more of the provided agents is an antagonist. The antagonistic activity of a disclosed agent may either be reversible or irreversible, and competitive or noncompetitive, depending on the mechanism by which the antagonist binds. In some embodiments, an “antagonist” may refer to a substance that inhibits the receptor(s) to which it binds with an IC₅₀ that is 50 micromolar or less, 40 micromolar or less, 30 micromolar or less, 20 micromolar or less, 10 micromolar or less, 5 micromolar or less, 2 micromolar or less, 1 micromolar or less, 500 nanomolar or less, 200 nanomolar or less, 100 nanomolar or less, 10 nanomolar or less, and values in between.

In some embodiments, an “antagonist” is at least 10-fold, at least 20-fold, at least 30-fold, at least 40-fold, at least 50-fold, at least 60-fold, at least 70-fold, at least 80-fold, at least 90-fold, at least 100-fold, or greater than 100-fold selective, including values in between (as measured by functional or competition binding assays), at the target receptor when compared to receptors not part of the target receptor system.

In some embodiments, any of an I₁ agent, 5-HT₂ agent, 5-HT₇ agent, and/or CB₁ agent may be an antagonist, i.e., an I₁ antagonist, 5-HT₂ antagonist, 5-HT₇ antagonist, and/or CB₁ antagonist.

In some embodiments, one or more of the agents provided herein are indirect or physiological antagonists. In some embodiments, an indirect antagonist or physiological antagonist may refer to an agent that produces biological effects at one receptor that partially or fully blocks, dampens, i.e., “inhibits” or “attenuates,” prevents, or delays the biologic effects caused by an agonist bound to a separate receptor system. Without being bound by theory, one non-limiting example of such action includes epinephrine raising arterial pressure through vasoconstriction mediated by alpha-1 adrenergic receptor activation. Thus, without physically binding to, or “antagonizing” the histamine receptor, epinephrine exhibits antihistamine properties—making it a physiological antagonist of histamine.

In some embodiments, a disclosed agent is a physiological antagonist, wherein the compound binds to and modulates a receptor in a manner that antagonizes the effects of an agonist bound to a receptor system useful in the practice of the invention.

In some embodiments, a disclosed agent is a physiological antagonist of imidazoline-1, wherein the compound binds to and modulates a receptor in a manner that antagonizes the effects of an agonist bound to the I₁ receptor system.

In some embodiments, a disclosed agent is a physiological antagonist of cannabinoid-1, wherein the compound binds to and modulates a receptor in a manner that antagonizes the effects of an agonist bound to the CB₁ receptor system.

In some embodiments, a disclosed agent is a physiological antagonist of serotonin-2, wherein the compound binds to and modulates a receptor in a manner that antagonizes the effects of an agonist bound to the 5-HT₂ receptor system.

In some embodiments, a disclosed agent is a physiological antagonist of serotonin-7, wherein the compound binds to and modulates a receptor in a manner that antagonizes the effects of an agonist bound to the 5-HT₇ receptor system.

In some embodiments, a disclosed agent is a physiological antagonist of imidazoline-1 and cannabinoid-1, wherein the compound binds to and modulates a receptor in a manner that antagonizes the effects of an agonist bound to any of the I₁ and CB₁ receptor systems.

In some embodiments, a disclosed agent is a physiological antagonist of imidazoline-1 and serotonin-2, wherein the compound binds to and modulates a receptor in a manner that antagonizes the effects of an agonist bound to any of the I₁ and 5-HT₂ receptor systems.

In some embodiments, a disclosed agent is a physiological antagonist of imidazoline-1 and serotonin-7, wherein the compound binds to and modulates a receptor in a manner that antagonizes the effects of an agonist bound to any of the I₁ and 5-HT₇ receptor systems.

In some embodiments, a disclosed agent is a physiological antagonist of cannabinoid-1 and serotonin-2, wherein the compound binds to and modulates a receptor in a manner that antagonizes the effects of an agonist bound to any of the CB₁ and 5-HT₂ receptor systems.

In some embodiments, a disclosed agent is a physiological antagonist of cannabinoid-1 and serotonin-7, wherein the compound binds to and modulates a receptor in a manner that antagonizes the effects of an agonist bound to any of the CB₁ and 5-HT₇ receptor systems.

In some embodiments, a disclosed agent is a physiological antagonist of serotonin-2 and serotonin-7, wherein the compound binds to and modulates a receptor in a manner that antagonizes the effects of an agonist bound to any of the 5-HT₂ and 5-HT₇ receptor systems.

In some embodiments, a disclosed agent is a physiological antagonist of imidazoline-1, cannabinoid-1, and serotonin-2, wherein the compound binds to and modulates a receptor in a manner that antagonizes the effects of an agonist bound to any of the I₁, CB₁, and 5-HT₂ receptor systems.

In some embodiments, a disclosed agent is a physiological antagonist of imidazoline-1, cannabinoid-1, and serotonin-7, wherein the compound binds to and modulates a receptor in a manner that antagonizes the effects of an agonist bound to any of the I₁, CB₁, and 5-HT₇ receptor systems.

In some embodiments, a disclosed agent is a physiological antagonist of imidazoline-1, serotonin-2, and serotonin-7, wherein the compound binds to and modulates a receptor in a manner that antagonizes the effects of an agonist bound to any of the I₁, 5-HT₂, and 5-HT₇ receptor systems.

In some embodiments, a disclosed agent is a physiological antagonist of cannabinoid-1, serotonin-2, and serotonin-7, wherein the compound binds to and modulates a receptor in a manner that antagonizes the effects of an agonist bound to any of the CB₁, 5-HT₂, and 5-HT₇ receptor systems.

In embodiments, a disclosed agent is a physiological antagonist of imidazoline-1, cannabinoid-1, serotonin-2, and serotonin-7, wherein the compound binds to and modulates a receptor in a manner that antagonizes the effects of an agonist bound to any of the I₁, CB₁, 5-HT₂, and 5-HT₇ receptor systems.

In some embodiments, an antagonist may be distinguished by its selectivity for a given receptor or set of receptors. In such embodiments, a compound is an antagonist of at least one desired receptor (i.e., any of the I₁, 5-HT₂, 5-HT₇, or CB₁ receptors, or a combination thereof), and possesses a selectivity for the at least one desired receptor that is greater than a ratio of 1.0. Meaning, the ratio of a compound's selectivity (as measured via Kᵢ) for at least one desired receptor system is greater than 1.1:1, 1.5:1, 2:1, 5:1, 10:1, 15:1, 25:1, 50:1, 100:1, etc., as compared to receptors not part of at least one desired receptor system

iii. Selective Agonism

In some embodiments, whether a compound is a selective agonist may be measured by activity or efficacy (e.g., as EC₅₀), instead of binding affinity, for example in some embodiments as a measure to determine the selective agonist activity of compounds, and that a compound that selectively binds to one receptor and not another may be considered a selective agonist in that regard.

In embodiments, a compound suitable for the invention herein may be a selective agonist for at least one of I₁, CB₁, 5-HT₂, and 5-HT₇, wherein the compound possesses an EC₅₀ for at least one of I₁, CB₁, 5-HT₂, and 5-HT₇ as measured by a binding or functional assay, that is at least a factor of 2, at least a factor of 3, at least a factor of 4, at least a factor 5, at least a factor of 6, at least a factor of 7, at least a factor of 8, at least a factor of 9, at least a factor of 10, at least a factor of 11, at least a factor of 12, at least a factor of 20, at least a factor of 30, at least a factor of 50, at least a factor of 100, at least a factor of 200, at least a factor of 500, and values in between, greater than receptors not part of the I₁, CB₁, 5-HT₂, and 5-HT₇ receptor systems.

In embodiments, a disclosed agent is a selective agonist for I₁, wherein the compound possesses an EC₅₀ for I₁ as measured by a binding or functional assay, that is at least a factor of 2, at least a factor of 3, at least a factor of 4, at least a factor 5, at least a factor of 6, at least a factor of 7, at least a factor of 8, at least a factor of 9, at least a factor of 10, at least a factor of 11, at least a factor of 12, at least a factor of 20, at least a factor of 30, at least a factor of 50, at least a factor of 100, at least a factor of 200, at least a factor of 500, and values in between, greater than receptors not part of the I₁ receptor system.

In embodiments, a disclosed agent is a selective agonist for CB₁, wherein the compound possesses an EC₅₀ for CB₁ as measured by a binding or functional assay, that is at least a factor of 2, at least a factor of 3, at least a factor of 4, at least a factor 5, at least a factor of 6, at least a factor of 7, at least a factor of 8, at least a factor of 9, at least a factor of 10, at least a factor of 11, at least a factor of 12, at least a factor of 20, at least a factor of 30, at least a factor of 50, at least a factor of 100, at least a factor of 200, at least a factor of 500, and values in between, greater than receptors not part of the CB₁ receptor system.

In embodiments, a disclosed agent is a selective agonist for 5-HT₂, wherein the compound possesses an EC₅₀ for 5-HT₂ as measured by a binding or functional assay, that is at least a factor of 2, at least a factor of 3, at least a factor of 4, at least a factor 5, at least a factor of 6, at least a factor of 7, at least a factor of 8, at least a factor of 9, at least a factor of 10, at least a factor of 11, at least a factor of 12, at least a factor of 20, at least a factor of 30, at least a factor of 50, at least a factor of 100, at least a factor of 200, at least a factor of 500, and values in between, greater than receptors not part of the 5-HT₂ receptor system.

In embodiments, a disclosed agent is a selective agonist for 5-HT₇, wherein the compound possesses an EC₅₀ for 5-HT₇ as measured by a binding or functional assay, that is at least a factor of 2, at least a factor of 3, at least a factor of 4, at least a factor 5, at least a factor of 6, at least a factor of 7, at least a factor of 8, at least a factor of 9, at least a factor of 10, at least a factor of 11, at least a factor of 12, at least a factor of 20, at least a factor of 30, at least a factor of 50, at least a factor of 100, at least a factor of 200, at least a factor of 500, and values in between, greater than receptors not part of the 5-HT₇ receptor system.

In embodiments, a disclosed agent is a selective agonist for imidazoline-1 and cannabinoid-1, wherein the compound possesses an EC₅₀ for imidazoline-1 and cannabinoid-1 as measured by a binding or functional assay, that is at least a factor of 2, at least a factor of 3, at least a factor of 4, at least a factor 5, at least a factor of 6, at least a factor of 7, at least a factor of 8, at least a factor of 9, at least a factor of 10, at least a factor of 11, at least a factor of 12, at least a factor of 20, at least a factor of 30, at least a factor of 50, at least a factor of 100, at least a factor of 200, at least a factor of 500, and values in between, greater than receptors not part of the I₁ or CB₁ receptor systems.

In embodiments, a disclosed agent is a selective agonist for imidazoline-1 and serotonin-2, wherein the compound possesses an EC₅₀ for imidazoline-1 and serotonin-2 as measured by a binding or functional assay, that is at least a factor of 2, at least a factor of 3, at least a factor of 4, at least a factor 5, at least a factor of 6, at least a factor of 7, at least a factor of 8, at least a factor of 9, at least a factor of 10, at least a factor of 11, at least a factor of 12, at least a factor of 20, at least a factor of 30, at least a factor of 50, at least a factor of 100, at least a factor of 200, at least a factor of 500, and values in between, greater than receptors not part of the I₁ or 5-HT₂ receptor systems.

In embodiments, a disclosed agent is a selective agonist for imidazoline-1 and serotonin-7, wherein the compound possesses an EC₅₀ for imidazoline-1 and serotonin-7 as measured by a binding or functional assay, that is at least a factor of 2, at least a factor of 3, at least a factor of 4, at least a factor 5, at least a factor of 6, at least a factor of 7, at least a factor of 8, at least a factor of 9, at least a factor of 10, at least a factor of 11, at least a factor of 12, at least a factor of 20, at least a factor of 30, at least a factor of 50, at least a factor of 100, at least a factor of 200, at least a factor of 500, and values in between, greater than receptors not part of the I₁ or 5-HT₇ receptor systems.

In embodiments, a disclosed agent is a selective agonist for cannabinoid-1 and serotonin-2, wherein the compound possesses an EC₅₀ for cannabinoid-1 and serotonin-2 as measured by a binding or functional assay, that is at least a factor of 2, at least a factor of 3, at least a factor of 4, at least a factor 5, at least a factor of 6, at least a factor of 7, at least a factor of 8, at least a factor of 9, at least a factor of 10, at least a factor of 11, at least a factor of 12, at least a factor of 20, at least a factor of 30, at least a factor of 50, at least a factor of 100, at least a factor of 200, at least a factor of 500, and values in between, greater than receptors not part of the CB₁ or 5-HT₂ receptor systems.

In embodiments, a disclosed agent is a selective agonist for cannabinoid-1 and serotonin-7, wherein the compound possesses an EC₅₀ for cannabinoid-1 and serotonin-7 as measured by a binding or functional assay, that is at least a factor of 2, at least a factor of 3, at least a factor of 4, at least a factor 5, at least a factor of 6, at least a factor of 7, at least a factor of 8, at least a factor of 9, at least a factor of 10, at least a factor of 11, at least a factor of 12, at least a factor of 20, at least a factor of 30, at least a factor of 50, at least a factor of 100, at least a factor of 200, at least a factor of 500, and values in between, greater than receptors not part of the CB₁ or 5-HT₇ receptor systems.

In embodiments, a disclosed agent is a selective agonist for serotonin-2 and serotonin-7, wherein the compound possesses an EC₅₀ for serotonin-2 and serotonin-7 as measured by a binding or functional assay, that is at least a factor of 2, at least a factor of 3, at least a factor of 4, at least a factor 5, at least a factor of 6, at least a factor of 7, at least a factor of 8, at least a factor of 9, at least a factor of 10, at least a factor of 11, at least a factor of 12, at least a factor of 20, at least a factor of 30, at least a factor of 50, at least a factor of 100, at least a factor of 200, at least a factor of 500, and values in between, greater than receptors not part of the 5-HT₂ or 5-HT₇ receptor systems.

In embodiments, a disclosed agent is a selective agonist for imidazoline-1, cannabinoid-1, and serotonin-2, wherein the compound possesses an EC₅₀ for imidazoline-1, cannabinoid-1, and serotonin-2 as measured by a binding or functional assay, that is at least a factor of 2, at least a factor of 3, at least a factor of 4, at least a factor 5, at least a factor of 6, at least a factor of 7, at least a factor of 8, at least a factor of 9, at least a factor of 10, at least a factor of 11, at least a factor of 12, at least a factor of 20, at least a factor of 30, at least a factor of 50, at least a factor of 100, at least a factor of 200, at least a factor of 500, and values in between, greater than receptors not part of the I₁, CB₁, or 5-HT₂ receptor systems.

In embodiments, a disclosed agent is a selective agonist for imidazoline-1, cannabinoid-1, and serotonin-7, wherein the compound possesses an EC₅₀ for imidazoline-1, cannabinoid-1, and serotonin-7 as measured by a binding or functional assay, that is at least a factor of 2, at least a factor of 3, at least a factor of 4, at least a factor 5, at least a factor of 6, at least a factor of 7, at least a factor of 8, at least a factor of 9, at least a factor of 10, at least a factor of 11, at least a factor of 12, at least a factor of 20, at least a factor of 30, at least a factor of 50, at least a factor of 100, at least a factor of 200, at least a factor of 500, and values in between, greater than receptors not part of the I₁, CB₁, or 5-HT₇ receptor systems.

In embodiments, a disclosed agent is a selective agonist for imidazoline-1, serotonin-2, and serotonin-7, wherein the compound possesses an affinity for imidazoline-1, serotonin-2, and serotonin-7 as measured by a binding or functional assay, that is at least a factor of 2, at least a factor of 3, at least a factor of 4, at least a factor 5, at least a factor of 6, at least a factor of 7, at least a factor of 8, at least a factor of 9, at least a factor of 10, at least a factor of 11, at least a factor of 12, at least a factor of 20, at least a factor of 30, at least a factor of 50, at least a factor of 100, at least a factor of 200, at least a factor of 500, and values in between, greater than receptors not part of the I₁, 5-HT₂, or 5-HT₇ receptor systems.

In embodiments, a disclosed agent is a selective agonist for cannabinoid-1, serotonin-2, and serotonin-7, wherein the compound possesses an affinity for cannabinoid-1, serotonin-2, and serotonin-7 as measured by a binding or functional assay, that is at least a factor of 2, at least a factor of 3, at least a factor of 4, at least a factor 5, at least a factor of 6, at least a factor of 7, at least a factor of 8, at least a factor of 9, at least a factor of 10, at least a factor of 11, at least a factor of 12, at least a factor of 20, at least a factor of 30, at least a factor of 50, at least a factor of 100, at least a factor of 200, at least a factor of 500, and values in between, greater than receptors not part of the CB₁, 5-HT₂, or 5-HT₇ receptor systems.

In embodiments, a disclosed agent is a selective agonist for imidazoline-1, cannabinoid-1, serotonin-2, and serotonin-7, wherein the compound possesses an affinity for imidazoline-1, cannabinoid-1, serotonin-2, and serotonin-7 as measured by a binding or functional assay, that is at least a factor of 2, at least a factor of 3, at least a factor of 4, at least a factor 5, at least a factor of 6, at least a factor of 7, at least a factor of 8, at least a factor of 9, at least a factor of 10, at least a factor of 11, at least a factor of 12, at least a factor of 20, at least a factor of 30, at least a factor of 50, at least a factor of 100, at least a factor of 200, at least a factor of 500, and values in between, greater than receptors not part of the I₁, CB₁, 5-HT₂, or 5-HT₇ receptor systems.

In some preferred embodiments, a disclosed agent is an agonist selective for imidazoline-1 over alpha-2, wherein the compound possesses an affinity for imidazoline-1 that is at least a factor of 2, including a factor of 3, a factor of 4, a factor 5, a factor of 6, a factor of 7, a factor of 8, a factor of 9, a factor of 10, a factor of 11, a factor of 12, a factor of 20, a factor of 30, a factor of 50, a factor of 100, a factor of 200, a factor of 500, and values in between, greater than that of alpha-2 adrenergic receptors. In such embodiments, the I₁ agonist may also be selective for at least one of the 5-HT₂, 5-HT₇, and CB₁ receptor systems.

In some preferred embodiments, a disclosed agent is an agonist selective for serotonin-2A and serotonin-2C over serotonin-2B, wherein the compound possesses an affinity for serotonin-2A and serotonin-2C that is at least a factor of 2, including a factor of 3, a factor of 4, a factor 5, a factor of 6, a factor of 7, a factor of 8, a factor of 9, a factor of 10, a factor of 11, a factor of 12, a factor of 20, a factor of 30, a factor of 50, a factor of 100, a factor of 200, a factor of 500, and values in between, greater than that of serotonin-2B receptors. In such embodiments, the 5-HT₂ agonist may also be selective for at least one of the I₁, 5-HT₇, and CB₁ receptor systems.

iv. Selective Antagonism

In embodiments, the agents provided herein are selective antagonists. In embodiments, a selective antagonist possesses an IC₅₀ at its target receptor at least a factor of 2, including a factor of 3, a factor of 4, a factor 5, a factor of 6, a factor of 7, a factor of 8, a factor of 9, a factor of 10, a factor of 11, a factor of 12, a factor of 20, a factor of 30, a factor of 50, a factor of 100, a factor of 200, a factor of 500, and values in between, lower than its IC₅₀ for receptors other than the target receptor.

In embodiments, a compound suitable for the invention herein may be a selective antagonist for at least one of imidazoline-1, cannabinoid-1, serotonin-2, and serotonin-7, wherein the compound possesses an IC₅₀ for at least one of imidazoline-1, cannabinoid-1, serotonin-2, or serotonin-7 as measured by a binding or functional assay, that is at least a factor of 2, including a factor of 3, a factor of 4, a factor 5, a factor of 6, a factor of 7, a factor of 8, a factor of 9, a factor of 10, a factor of 11, a factor of 12, a factor of 20, a factor of 30, a factor of 50, a factor of 100, a factor of 200, a factor of 500, and values in between, greater than receptors not part of the I₁, CB₁, 5-HT₂, and 5-HT₇ receptor systems receptor systems.

In embodiments, a disclosed agent is a selective antagonist for imidazoline-1, wherein the compound possesses an IC₅₀ for imidazoline-1 that is at least a factor of 2, including a factor of 3, a factor of 4, a factor 5, a factor of 6, a factor of 7, a factor of 8, a factor of 9, a factor of 10, a factor of 11, a factor of 12, a factor of 20, a factor of 30, a factor of 50, a factor of 100, a factor of 200, a factor of 500, and values in between, lower than its IC₅₀ for receptors not part of the I₁ receptor system.

In embodiments, a disclosed agent is a selective antagonist for serotonin-2, wherein the compound possesses an IC₅₀ for serotonin-2 that is at least a factor of 2, including a factor of 3, a factor of 4, a factor 5, a factor of 6, a factor of 7, a factor of 8, a factor of 9, a factor of 10, a factor of 11, a factor of 12, a factor of 20, a factor of 30, a factor of 50, a factor of 100, a factor of 200, a factor of 500, and values in between, lower than its IC₅₀ for receptors not part of the 5-HT₂ receptor system.

In embodiments, a disclosed agent is a selective antagonist for serotonin-7, wherein the compound possesses an IC₅₀ for serotonin-7 that is at least a factor of 2, including a factor of 3, a factor of 4, a factor 5, a factor of 6, a factor of 7, a factor of 8, a factor of 9, a factor of 10, a factor of 11, a factor of 12, a factor of 20, a factor of 30, a factor of 50, a factor of 100, a factor of 200, a factor of 500, and values in between, lower than its IC₅₀ for receptors not part of the 5-HT₇ receptor system.

In embodiments, a disclosed agent is a selective antagonist for serotonin-2 and serotonin-7, wherein the compound possesses an IC₅₀ for serotonin-7 that is at least a factor of 2, including a factor of 3, a factor of 4, a factor 5, a factor of 6, a factor of 7, a factor of 8, a factor of 9, a factor of 10, a factor of 11, a factor of 12, a factor of 20, a factor of 30, a factor of 50, a factor of 100, a factor of 200, a factor of 500, and values in between, lower than its IC₅₀ for receptors not part of the 5-HT₂ or 5-HT₇ receptor systems.

In embodiments, a disclosed agent is a selective antagonist for CB₁, wherein the compound possesses an IC₅₀ for cannabinoid-1 that is at least a factor of 2, including a factor of 3, a factor of 4, a factor 5, a factor of 6, a factor of 7, a factor of 8, a factor of 9, a factor of 10, a factor of 11, a factor of 12, a factor of 20, a factor of 30, a factor of 50, a factor of 100, a factor of 200, a factor of 500, and values in between, lower than its IC₅₀ for receptors not part of the CB₁ receptor system.

In embodiments, a disclosed agent is a selective antagonist for I₁ and CB₁, wherein the compound possesses an IC₅₀ for I₁ and CB₁ that is at least a factor of 2, including a factor of 3, a factor of 4, a factor 5, a factor of 6, a factor of 7, a factor of 8, a factor of 9, a factor of 10, a factor of 11, a factor of 12, a factor of 20, a factor of 30, a factor of 50, a factor of 100, a factor of 200, a factor of 500, and values in between, lower than its IC₅₀ for receptors not part of the I₁ or CB₁ receptor systems.

In embodiments, a disclosed agent is a selective antagonist for I₁ and 5-HT₂, wherein the compound possesses an IC₅₀ for I₁ and 5-HT₂ that is at least a factor of 2, including a factor of 3, a factor of 4, a factor 5, a factor of 6, a factor of 7, a factor of 8, a factor of 9, a factor of 10, a factor of 11, a factor of 12, a factor of 20, a factor of 30, a factor of 50, a factor of 100, a factor of 200, a factor of 500, and values in between, lower than its IC₅₀ for receptors not part of the I₁ or 5-HT₂ receptor systems.

In embodiments, a disclosed agent is a selective antagonist for I₁ and 5-HT₇, wherein the compound possesses an IC₅₀ for I₁ and 5-HT₇ that is at least a factor of 2, including a factor of 3, a factor of 4, a factor 5, a factor of 6, a factor of 7, a factor of 8, a factor of 9, a factor of 10, a factor of 11, a factor of 12, a factor of 20, a factor of 30, a factor of 50, a factor of 100, a factor of 200, a factor of 500, and values in between, lower than its IC₅₀ for receptors not part of the I₁ or 5-HT₇ receptor systems.

In embodiments, a disclosed agent is a selective antagonist for imidazoline-1, serotonin-2, and serotonin-7, wherein the compound possesses an IC₅₀ for imidazoline-1, serotonin-2, and serotonin-7 that is at least a factor of 2, including a factor of 3, a factor of 4, a factor 5, a factor of 6, a factor of 7, a factor of 8, a factor of 9, a factor of 10, a factor of 11, a factor of 12, a factor of 20, a factor of 30, a factor of 50, a factor of 100, a factor of 200, a factor of 500, and values in between, lower than its IC₅₀ for receptors not part of the I₁, 5-HT₂, or 5-HT₇ receptor systems.

In embodiments, a disclosed agent is a selective antagonist for imidazoline-1, cannabinoid-1, and serotonin-2, wherein the compound possesses an IC₅₀ for imidazoline-1, cannabinoid-1, and serotonin-2 that is at least a factor of 2, including a factor of 3, a factor of 4, a factor 5, a factor of 6, a factor of 7, a factor of 8, a factor of 9, a factor of 10, a factor of 11, a factor of 12, a factor of 20, a factor of 30, a factor of 50, a factor of 100, a factor of 200, a factor of 500, and values in between, lower than its IC₅₀ for receptors not part of the I₁, CB₁, or 5-HT₂ receptor systems.

In embodiments, a disclosed agent is a selective antagonist for imidazoline-1, cannabinoid-1, and serotonin-7, wherein the compound possesses an IC₅₀ for imidazoline-1, cannabinoid-1, and serotonin-7 that is at least a factor of 2, including a factor of 3, a factor of 4, a factor 5, a factor of 6, a factor of 7, a factor of 8, a factor of 9, a factor of 10, a factor of 11, a factor of 12, a factor of 20, a factor of 30, a factor of 50, a factor of 100, a factor of 200, a factor of 500, and values in between, lower than its IC₅₀ for receptors not part of the I₁, CB₁, or 5-HT₇ receptor systems.

In embodiments, a disclosed agent is a selective antagonist for imidazoline-1, cannabinoid-1, serotonin-2, and serotonin-7, wherein the compound possesses an IC₅₀ for imidazoline-1, cannabinoid-1, serotonin-2, and serotonin-7 that is at least a factor of 2, including a factor of 3, a factor of 4, a factor 5, a factor of 6, a factor of 7, a factor of 8, a factor of 9, a factor of 10, a factor of 11, a factor of 12, a factor of 20, a factor of 30, a factor of 50, a factor of 100, a factor of 200, a factor of 500, and values in between, lower than its IC₅₀ for receptors not part of the I₁, CB₁, 5-HT₂, or 5-HT₇ receptor systems.

In embodiments, a disclosed agent is a selective antagonist for CB₁ and 5-HT₂, wherein the compound possesses an IC₅₀ for CB₁ and 5-HT₂ that is at least a factor of 2, including a factor of 3, a factor of 4, a factor 5, a factor of 6, a factor of 7, a factor of 8, a factor of 9, a factor of 10, a factor of 11, a factor of 12, a factor of 20, a factor of 30, a factor of 50, a factor of 100, a factor of 200, a factor of 500, and values in between, lower than its IC₅₀ for receptors not part of the CB₁ or 5-HT₂ receptor systems.

In embodiments, a disclosed agent is a selective antagonist for CB₁ and 5-HT₇, wherein the compound possesses an IC₅₀ for CB₁ and 5-HT₇ that is at least a factor of 2, including a factor of 3, a factor of 4, a factor 5, a factor of 6, a factor of 7, a factor of 8, a factor of 9, a factor of 10, a factor of 11, a factor of 12, a factor of 20, a factor of 30, a factor of 50, a factor of 100, a factor of 200, a factor of 500, and values in between, lower than its IC₅₀ for receptors not part of the CB₁ or 5-HT₇ receptor systems.

In embodiments, a disclosed agent is a selective antagonist for cannabinoid-1, serotonin-2, and serotonin-7, wherein the compound possesses an IC₅₀ for cannabinoid-1, serotonin-2, and serotonin-7 that is at least a factor of 2, including a factor of 3, a factor of 4, a factor 5, a factor of 6, a factor of 7, a factor of 8, a factor of 9, a factor of 10, a factor of 11, a factor of 12, a factor of 20, a factor of 30, a factor of 50, a factor of 100, a factor of 200, a factor of 500, and values in between, lower than its IC₅₀ for receptors not part of the CB₁, 5-HT₂, or 5-HT₇ receptor systems.

### G. Exemplary Therapeutic Combinations

In embodiments, a therapeutic combination of the invention comprises an I₁ agent and a CB₁ agent. In embodiments, a therapeutic combination of the invention comprises an I₁ agonist and a CB₁ agonist. In embodiments, a therapeutic combination of the invention comprises an I₁ agonist and a CB₁ antagonist. In embodiments, a therapeutic combination of the invention comprises an I₁ antagonist and a CB₁ agonist. In embodiments, a therapeutic combination of the invention comprises an I₁ antagonist and a CB₁ antagonist.

In embodiments, a therapeutic combination of the invention comprises an I₁ agent and a 5-HT₂ agent. In embodiments, a therapeutic combination of the invention comprises an I₁ agonist and a 5-HT₂ agonist. In embodiments, a therapeutic combination of the invention comprises an I₁ agonist and a 5-HT₂ antagonist. In embodiments, a therapeutic combination of the invention comprises an I₁ antagonist and a 5-HT₂ agonist. In embodiments, a therapeutic combination of the invention comprises an I₁ antagonist and a 5-HT₂ antagonist.

In embodiments, a therapeutic combination of the invention comprises an I₁ agent and a 5-HT₇ agent. In embodiments, a therapeutic combination of the invention comprises an I₁ agonist and a 5-HT₇ agonist. In embodiments, a therapeutic combination of the invention comprises an I₁ agonist and a 5-HT₇ antagonist. In embodiments, a therapeutic combination of the invention comprises an I₁ antagonist and a 5-HT₇ agonist. In embodiments, a therapeutic combination of the invention comprises an I₁ antagonist and a 5-HT₇ antagonist.

In embodiments, a therapeutic combination of the invention comprises an I₁ agent, a CB₁ agent, and a 5-HT₂ agent. In embodiments, a therapeutic combination of the invention comprises an I₁ agonist, a CB₁ agonist, and a 5-HT₂ agonist. In embodiments, a therapeutic combination of the invention comprises an I₁ agonist, a CB₁ agonist, and a 5-HT₂ antagonist. In embodiments, a therapeutic combination of the invention comprises an I₁ agonist, a CB₁ antagonist, and a 5-HT₂ agonist. In embodiments, a therapeutic combination of the invention comprises an I₁ agonist, a CB₁ antagonist, and a 5-HT₂ antagonist. In embodiments, a therapeutic combination of the invention comprises an I₁ agent, a CB₁ agent, and a 5-HT₂ agent. In embodiments, a therapeutic combination of the invention comprises an I₁ antagonist, a CB₁ agonist, and a 5-HT₂ agonist. In embodiments, a therapeutic combination of the invention comprises an I₁ antagonist, a CB₁ agonist, and a 5-HT₂ antagonist. In embodiments, a therapeutic combination of the invention comprises an I₁ antagonist, a CB₁ antagonist, and a 5-HT₂ agonist. In embodiments, a therapeutic combination of the invention comprises an I₁ antagonist, a CB₁ antagonist, and a 5-HT₂ antagonist.

In embodiments, a therapeutic combination of the invention comprises an I₁ agent, a CB₁ agent, and a 5-HT₇ agent. In embodiments, a therapeutic combination of the invention comprises an I₁ agonist, a CB₁ agonist, and a 5-HT₇ agonist. In embodiments, a therapeutic combination of the invention comprises an I₁ agonist, a CB₁ agonist, and a 5-HT₇ antagonist. In embodiments, a therapeutic combination of the invention comprises an I₁ agonist, a CB₁ antagonist, and a 5-HT₇ agonist. In embodiments, a therapeutic combination of the invention comprises an I₁ agonist, a CB₁ antagonist, and a 5-HT₇ antagonist. In embodiments, a therapeutic combination of the invention comprises an I₁ agent, a CB₁ agent, and a 5-HT₇ agent. In embodiments, a therapeutic combination of the invention comprises an I₁ antagonist, a CB₁ agonist, and a 5-HT₇ agonist. In embodiments, a therapeutic combination of the invention comprises an I₁ antagonist, a CB₁ agonist, and a 5-HT₇ antagonist. In embodiments, a therapeutic combination of the invention comprises an I₁ antagonist, a CB₁ antagonist, and a 5-HT₇ agonist. In embodiments, a therapeutic combination of the invention comprises an I₁ antagonist, a CB₁ antagonist, and a 5-HT₇ antagonist.

In embodiments, a therapeutic combination of the invention comprises an I₁ agent, a 5-HT₂ agent, and a 5-HT₇ agent. In embodiments, a therapeutic combination of the invention comprises an I₁ agonist, a 5-HT₂ agonist, and a 5-HT₇ agonist. In embodiments, a therapeutic combination of the invention comprises an I₁ agonist, a 5-HT₂ agonist, and a 5-HT₇ antagonist. In embodiments, a therapeutic combination of the invention comprises an I₁ agonist, a 5-HT₂ antagonist, and a 5-HT₇ agonist. In embodiments, a therapeutic combination of the invention comprises an I₁ agonist, a 5-HT₂ antagonist, and a 5-HT₇ antagonist. In embodiments, a therapeutic combination of the invention comprises an I₁ agent, a 5-HT₂ agent, and a 5-HT₇ agent. In embodiments, a therapeutic combination of the invention comprises an I₁ antagonist, a 5-HT₂ agonist, and a 5-HT₇ agonist. In embodiments, a therapeutic combination of the invention comprises an I₁ antagonist, a 5-HT₂ agonist, and a 5-HT₇ antagonist. In embodiments, a therapeutic combination of the invention comprises an I₁ antagonist, a 5-HT₂ antagonist, and a 5-HT₇ agonist. In embodiments, a therapeutic combination of the invention comprises an I₁ antagonist, a 5-HT₂ antagonist, and a 5-HT₇ antagonist.

In embodiments, a therapeutic combination of the invention comprises an I₁ agent, a CB₁ agent, a 5-HT₂ agent, and a 5-HT₇ agent. In embodiments, a therapeutic combination of the invention comprises an I₁ agonist, a CB₁ agonist, a 5-HT₂ agonist, and a 5-HT₇ agonist. In embodiments, a therapeutic combination of the invention comprises an I₁ agonist, a CB₁ agonist, a 5-HT₂ agonist, and a 5-HT₇ antagonist. In embodiments, a therapeutic combination of the invention comprises an I₁ agonist, a CB₁ agonist, a 5-HT₂ antagonist, and a 5-HT₇ agonist. In embodiments, a therapeutic combination of the invention comprises an I₁ agonist, a CB₁ agonist, a 5-HT₂ antagonist, and a 5-HT₇ antagonist. In embodiments, a therapeutic combination of the invention comprises an I₁ agonist, a CB₁ antagonist, a 5-HT₂ agonist, and a 5-HT₇ agonist. In embodiments, a therapeutic combination of the invention comprises an I₁ agonist, a CB₁ antagonist, a 5-HT₂ agonist, and a 5-HT₇ antagonist. In embodiments, a therapeutic combination of the invention comprises an I₁ agonist, a CB₁ antagonist, a 5-HT₂ antagonist, and a 5-HT₇ agonist. In embodiments, a therapeutic combination of the invention comprises an I₁ agonist, a CB₁ antagonist, a 5-HT₂ antagonist, and a 5-HT₇ antagonist. In embodiments, a therapeutic combination of the invention comprises an I₁ antagonist, a CB₁ agonist, a 5-HT₂ agonist, and a 5-HT₇ agonist. In embodiments, a therapeutic combination of the invention comprises an I₁ antagonist, a CB₁ agonist, a 5-HT₂ agonist, and a 5-HT₇ antagonist. In embodiments, a therapeutic combination of the invention comprises an I₁ antagonist, a CB₁ agonist, a 5-HT₂ antagonist, and a 5-HT₇ agonist. In embodiments, a therapeutic combination of the invention comprises an I₁ antagonist, a CB₁ agonist, a 5-HT₂ antagonist, and a 5-HT₇ antagonist. In embodiments, a therapeutic combination of the invention comprises an I₁ antagonist, a CB₁ antagonist, a 5-HT₂ agonist, and a 5-HT₇ agonist. In embodiments, a therapeutic combination of the invention comprises an I₁ antagonist, a CB₁ antagonist, a 5-HT₂ agonist, and a 5-HT₇ antagonist. In embodiments, a therapeutic combination of the invention comprises an I₁ antagonist, a CB₁ antagonist, a 5-HT₂ antagonist, and a 5-HT₇ agonist. In embodiments, a therapeutic combination of the invention comprises an I₁ antagonist, a CB₁ antagonist, a 5-HT₂ antagonist, and a 5-HT₇ antagonist.

In embodiments, a therapeutic combination of the invention comprises an I₁ agent and a CB₁ agent administered together but not formulated as part of a single pharmaceutical composition; in other embodiments, a therapeutic combination of the invention comprises an I₁ agent and a CB₁ agent, wherein the combination is formulated as a single pharmaceutical composition, further comprising a pharmaceutically acceptable carrier, diluent, or excipient. In embodiments, a therapeutic combination of the invention comprises an I₁ agonist and a CB₁ agonist, wherein the combination is formulated as a single pharmaceutical composition, further comprising a pharmaceutically acceptable carrier, diluent, or excipient. In embodiments, a therapeutic combination of the invention comprises an I₁ agonist and a CB₁ antagonist, wherein the combination is formulated as a single pharmaceutical composition, further comprising a pharmaceutically acceptable carrier, diluent, or excipient. In embodiments, a therapeutic combination of the invention comprises an I₁ antagonist and a CB₁ agonist, wherein the combination is formulated as a single pharmaceutical composition, further comprising a pharmaceutically acceptable carrier, diluent, or excipient. In embodiments, a therapeutic combination of the invention comprises an I₁ antagonist and a CB₁ antagonist, wherein the combination is formulated as a single pharmaceutical composition, further comprising a pharmaceutically acceptable carrier, diluent, or excipient.

In embodiments, a therapeutic combination of the invention comprises an I₁ agent and a 5-HT₂ agent administered together but not formulated as part of a single pharmaceutical composition; in other embodiments, a therapeutic combination of the invention comprises an I₁ agent and a 5-HT₂ agent, wherein the combination is formulated as a single pharmaceutical composition, further comprising a pharmaceutically acceptable carrier, diluent, or excipient. In embodiments, a therapeutic combination of the invention comprises an I₁ agonist and a 5-HT₂ agonist, wherein the combination is formulated as a single pharmaceutical composition, further comprising a pharmaceutically acceptable carrier, diluent, or excipient. In embodiments, a therapeutic combination of the invention comprises an I₁ agonist and a 5-HT₂ antagonist, wherein the combination is formulated as a single pharmaceutical composition, further comprising a pharmaceutically acceptable carrier, diluent, or excipient. In embodiments, a therapeutic combination of the invention comprises an I₁ antagonist and a 5-HT₂ agonist, wherein the combination is formulated as a single pharmaceutical composition, further comprising a pharmaceutically acceptable carrier, diluent, or excipient. In embodiments, a therapeutic combination of the invention comprises an I₁ antagonist and a 5-HT₂ antagonist, wherein the combination is formulated as a single pharmaceutical composition, further comprising a pharmaceutically acceptable carrier, diluent, or excipient.

In embodiments, a therapeutic combination of the invention comprises an I₁ agent and a 5-HT₇ agent administered together but not formulated as part of a single pharmaceutical composition; in other embodiments, a therapeutic combination of the invention comprises an I₁ agent and a 5-HT₇ agent, wherein the combination is formulated as a single pharmaceutical composition, further comprising a pharmaceutically acceptable carrier, diluent, or excipient. In embodiments, a therapeutic combination of the invention comprises an I₁ agonist and a 5-HT₇ agonist, wherein the combination is formulated as a single pharmaceutical composition, further comprising a pharmaceutically acceptable carrier, diluent, or excipient. In embodiments, a therapeutic combination of the invention comprises an I₁ agonist and a 5-HT₇ antagonist, wherein the combination is formulated as a single pharmaceutical composition, further comprising a pharmaceutically acceptable carrier, diluent, or excipient. In embodiments, a therapeutic combination of the invention comprises an I₁ antagonist and a 5-HT₇ agonist, wherein the combination is formulated as a single pharmaceutical composition, further comprising a pharmaceutically acceptable carrier, diluent, or excipient. In embodiments, a therapeutic combination of the invention comprises an I₁ antagonist and a 5-HT₇ antagonist, wherein the combination is formulated as a single pharmaceutical composition, further comprising a pharmaceutically acceptable carrier, diluent, or excipient.

In embodiments, a therapeutic combination of the invention comprises an I₁ agent, a CB₁ agent, and a 5-HT₂ agent administered together but not formulated as part of a single pharmaceutical composition; in other embodiments, a therapeutic combination of the invention comprises an I₁ agent, a CB₁ agent, and a 5-HT₂ agent, wherein the combination is formulated as a single pharmaceutical composition, further comprising a pharmaceutically acceptable carrier, diluent, or excipient. In embodiments, a therapeutic combination of the invention comprises an I₁ agonist, a CB₁ agonist, and a 5-HT₂ agonist, wherein the combination is formulated as a single pharmaceutical composition, further comprising a pharmaceutically acceptable carrier, diluent, or excipient. In embodiments, a therapeutic combination of the invention comprises an I₁ agonist, a CB₁ agonist, and a 5-HT₂ antagonist, wherein the combination is formulated as a single pharmaceutical composition, further comprising a pharmaceutically acceptable carrier, diluent, or excipient. In embodiments, a therapeutic combination of the invention comprises an I₁ agonist, a CB₁ antagonist, and a 5-HT₂ agonist, wherein the combination is formulated as a single pharmaceutical composition, further comprising a pharmaceutically acceptable carrier, diluent, or excipient. In embodiments, a therapeutic combination of the invention comprises an I₁ agonist, a CB₁ antagonist, and a 5-HT₂ antagonist, wherein the combination is formulated as a single pharmaceutical composition, further comprising a pharmaceutically acceptable carrier, diluent, or excipient. In embodiments, a therapeutic combination of the invention comprises an I₁ antagonist, a CB₁ agonist, and a 5-HT₂ agonist, wherein the combination is formulated as a single pharmaceutical composition, further comprising a pharmaceutically acceptable carrier, diluent, or excipient. In embodiments, a therapeutic combination of the invention comprises an I₁ antagonist, a CB₁ agonist, and a 5-HT₂ antagonist, wherein the combination is formulated as a single pharmaceutical composition, further comprising a pharmaceutically acceptable carrier, diluent, or excipient. In embodiments, a therapeutic combination of the invention comprises an I₁ antagonist, a CB₁ antagonist, and a 5-HT₂ agonist, wherein the combination is formulated as a single pharmaceutical composition, further comprising a pharmaceutically acceptable carrier, diluent, or excipient. In embodiments, a therapeutic combination of the invention comprises an I₁ antagonist, a CB₁ antagonist, and a 5-HT₂ antagonist, wherein the combination is formulated as a single pharmaceutical composition, further comprising a pharmaceutically acceptable carrier, diluent, or excipient.

In embodiments, a therapeutic combination of the invention comprises an I₁ agent, a CB₁ agent, and a 5-HT₇ agent administered together but not formulated as part of a single pharmaceutical composition; in other embodiments, a therapeutic combination of the invention comprises an I₁ agent, a CB₁ agent, and a 5-HT₇ agent, wherein the combination is formulated as a single pharmaceutical composition, further comprising a pharmaceutically acceptable carrier, diluent, or excipient. In embodiments, a therapeutic combination of the invention comprises an I₁ agonist, a CB₁ agonist, and a 5-HT₇ agonist, wherein the combination is formulated as a single pharmaceutical composition, further comprising a pharmaceutically acceptable carrier, diluent, or excipient. In embodiments, a therapeutic combination of the invention comprises an I₁ agonist, a CB₁ agonist, and a 5-HT₇ antagonist, wherein the combination is formulated as a single pharmaceutical composition, further comprising a pharmaceutically acceptable carrier, diluent, or excipient. In embodiments, a therapeutic combination of the invention comprises an I₁ agonist, a CB₁ antagonist, and a 5-HT₇ agonist, wherein the combination is formulated as a single pharmaceutical composition, further comprising a pharmaceutically acceptable carrier, diluent, or excipient. In embodiments, a therapeutic combination of the invention comprises an I₁ agonist, a CB₁ antagonist, and a 5-HT₇ antagonist, wherein the combination is formulated as a single pharmaceutical composition, further comprising a pharmaceutically acceptable carrier, diluent, or excipient. In embodiments, a therapeutic combination of the invention comprises an I₁ antagonist, a CB₁ agonist, and a 5-HT₇ agonist, wherein the combination is formulated as a single pharmaceutical composition, further comprising a pharmaceutically acceptable carrier, diluent, or excipient. In embodiments, a therapeutic combination of the invention comprises an I₁ antagonist, a CB₁ agonist, and a 5-HT₇ antagonist, wherein the combination is formulated as a single pharmaceutical composition, further comprising a pharmaceutically acceptable carrier, diluent, or excipient. In embodiments, a therapeutic combination of the invention comprises an I₁ antagonist, a CB₁ antagonist, and a 5-HT₇ agonist, wherein the combination is formulated as a single pharmaceutical composition, further comprising a pharmaceutically acceptable carrier, diluent, or excipient. In embodiments, a therapeutic combination of the invention comprises an I₁ antagonist, a CB₁ antagonist, and a 5-HT₇ antagonist, wherein the combination is formulated as a single pharmaceutical composition, further comprising a pharmaceutically acceptable carrier, diluent, or excipient.

In embodiments, a therapeutic combination of the invention comprises an I₁ agent, a 5-HT₂ agent, and a 5-HT₇ agent administered together but not formulated as part of a single pharmaceutical composition; in other embodiments, a therapeutic combination of the invention comprises an I₁ agent, a 5-HT₂ agent, and a 5-HT₇ agent, wherein the combination is formulated as a single pharmaceutical composition, further comprising a pharmaceutically acceptable carrier, diluent, or excipient. In embodiments, a therapeutic combination of the invention comprises an I₁ agonist, a 5-HT₂ agonist, and a 5-HT₇ agonist, wherein the combination is formulated as a single pharmaceutical composition, further comprising a pharmaceutically acceptable carrier, diluent, or excipient. In embodiments, a therapeutic combination of the invention comprises an I₁ agonist, a 5-HT₂ agonist, and a 5-HT₇ antagonist, wherein the combination is formulated as a single pharmaceutical composition, further comprising a pharmaceutically acceptable carrier, diluent, or excipient. In embodiments, a therapeutic combination of the invention comprises an I₁ agonist, a 5-HT₂ antagonist, and a 5-HT₇ agonist, wherein the combination is formulated as a single pharmaceutical composition, further comprising a pharmaceutically acceptable carrier, diluent, or excipient. In embodiments, a therapeutic combination of the invention comprises an I₁ agonist, a 5-HT₂ antagonist, and a 5-HT₇ antagonist, wherein the combination is formulated as a single pharmaceutical composition, further comprising a pharmaceutically acceptable carrier, diluent, or excipient. In embodiments, a therapeutic combination of the invention comprises an I₁ antagonist, a 5-HT₂ agonist, and a 5-HT₇ agonist, wherein the combination is formulated as a single pharmaceutical composition, further comprising a pharmaceutically acceptable carrier, diluent, or excipient. In embodiments, a therapeutic combination of the invention comprises an I₁ antagonist, a 5-HT₂ agonist, and a 5-HT₇ antagonist, wherein the combination is formulated as a single pharmaceutical composition, further comprising a pharmaceutically acceptable carrier, diluent, or excipient. In embodiments, a therapeutic combination of the invention comprises an I₁ antagonist, a 5-HT₂ antagonist, and a 5-HT₇ agonist, wherein the combination is formulated as a single pharmaceutical composition, further comprising a pharmaceutically acceptable carrier, diluent, or excipient. In embodiments, a therapeutic combination of the invention comprises an I₁ antagonist, a 5-HT₂ antagonist, and a 5-HT₇ antagonist, wherein the combination is formulated as a single pharmaceutical composition, further comprising a pharmaceutically acceptable carrier, diluent, or excipient.

In embodiments, a therapeutic combination of the invention comprises an I₁ agent, a CB₁ agent, a 5-HT₂ agent, and a 5-HT₇ agent administered together but not formulated as part of a single pharmaceutical composition; in other embodiments, a therapeutic combination of the invention comprises an I₁ agent, a CB₁ agent, a 5-HT₂ agent, and a 5-HT₇ agent, wherein the combination is formulated as a single pharmaceutical composition, further comprising a pharmaceutically acceptable carrier, diluent, or excipient. In embodiments, a therapeutic combination of the invention comprises an I₁ agonist, a CB₁ agonist, a 5-HT₂ agonist, and a 5-HT₇ agonist, wherein the combination is formulated as a single pharmaceutical composition, further comprising a pharmaceutically acceptable carrier, diluent, or excipient. In embodiments, a therapeutic combination of the invention comprises an I₁ agonist, a CB₁ agonist, a 5-HT₂ agonist, and a 5-HT₇ antagonist, wherein the combination is formulated as a single pharmaceutical composition, further comprising a pharmaceutically acceptable carrier, diluent, or excipient. In embodiments, a therapeutic combination of the invention comprises an I₁ agonist, a CB₁ agonist, a 5-HT₂ antagonist, and a 5-HT₇ agonist, wherein the combination is formulated as a single pharmaceutical composition, further comprising a pharmaceutically acceptable carrier, diluent, or excipient. In embodiments, a therapeutic combination of the invention comprises an I₁ agonist, a CB₁ agonist, a 5-HT₂ antagonist, and a 5-HT₇ antagonist, wherein the combination is formulated as a single pharmaceutical composition, further comprising a pharmaceutically acceptable carrier, diluent, or excipient. In embodiments, a therapeutic combination of the invention comprises an I₁ agonist, a CB₁ antagonist, a 5-HT₂ agonist, and a 5-HT₇ agonist, wherein the combination is formulated as a single pharmaceutical composition, further comprising a pharmaceutically acceptable carrier, diluent, or excipient. In embodiments, a therapeutic combination of the invention comprises an I₁ agonist, a CB₁ antagonist, a 5-HT₂ agonist, and a 5-HT₇ antagonist, wherein the combination is formulated as a single pharmaceutical composition, further comprising a pharmaceutically acceptable carrier, diluent, or excipient. In embodiments, a therapeutic combination of the invention comprises an I₁ agonist, a CB₁ antagonist, a 5-HT₂ antagonist, and a 5-HT₇ agonist, wherein the combination is formulated as a single pharmaceutical composition, further comprising a pharmaceutically acceptable carrier, diluent, or excipient. In embodiments, a therapeutic combination of the invention comprises an I₁ agonist, a CB₁ antagonist, a 5-HT₂ antagonist, and a 5-HT₇ antagonist, wherein the combination is formulated as a single pharmaceutical composition, further comprising a pharmaceutically acceptable carrier, diluent, or excipient. In embodiments, a therapeutic combination of the invention comprises an I₁ antagonist, a CB₁ agonist, a 5-HT₂ agonist, and a 5-HT₇ agonist, wherein the combination is formulated as a single pharmaceutical composition, further comprising a pharmaceutically acceptable carrier, diluent, or excipient. In embodiments, a therapeutic combination of the invention comprises an I₁ antagonist, a CB₁ agonist, a 5-HT₂ agonist, and a 5-HT₇ antagonist, wherein the combination is formulated as a single pharmaceutical composition, further comprising a pharmaceutically acceptable carrier, diluent, or excipient. In embodiments, a therapeutic combination of the invention comprises an I₁ antagonist, a CB₁ agonist, a 5-HT₂ antagonist, and a 5-HT₇ agonist, wherein the combination is formulated as a single pharmaceutical composition, further comprising a pharmaceutically acceptable carrier, diluent, or excipient. In embodiments, a therapeutic combination of the invention comprises an I₁ antagonist, a CB₁ agonist, a 5-HT₂ antagonist, and a 5-HT₇ antagonist, wherein the combination is formulated as a single pharmaceutical composition, further comprising a pharmaceutically acceptable carrier, diluent, or excipient. In embodiments, a therapeutic combination of the invention comprises an I₁ antagonist, a CB₁ antagonist, a 5-HT₂ agonist, and a 5-HT₇ agonist, wherein the combination is formulated as a single pharmaceutical composition, further comprising a pharmaceutically acceptable carrier, diluent, or excipient. In embodiments, a therapeutic combination of the invention comprises an I₁ antagonist, a CB₁ antagonist, a 5-HT₂ agonist, and a 5-HT₇ antagonist, wherein the combination is formulated as a single pharmaceutical composition, further comprising a pharmaceutically acceptable carrier, diluent, or excipient. In embodiments, a therapeutic combination of the invention comprises an I₁ antagonist, a CB₁ antagonist, a 5-HT₂ antagonist, and a 5-HT₇ agonist, wherein the combination is formulated as a single pharmaceutical composition, further comprising a pharmaceutically acceptable carrier, diluent, or excipient. In embodiments, a therapeutic combination of the invention comprises an I₁ antagonist, a CB₁ antagonist, a 5-HT₂ antagonist, and a 5-HT₇ antagonist, wherein the combination is formulated as a single pharmaceutical composition, further comprising a pharmaceutically acceptable carrier, diluent, or excipient.

### H. Exemplary Synergistic Combinations

In embodiments, “synergistic,” “synergistic effects,” or “synergy” will refer to a pharmaceutical composition or combination therapy (comprising at least two agents) that, when administered to a subject, produces effects—either mental, physical, physiological, or a combination thereof—that are not experienced by the patient when taking one of the at least two agents on its own. In embodiments, the effects will be that of an entactogenic experience or “mindstate,” as defined herein.

In embodiments, “synergistic” may refer to a pharmaceutical composition or combination therapy that is more effective than the additive effects of any two or more single agents. A synergistic effect, for example, permits the effective treatment of a disease using lower amounts (doses) of individual therapy. This includes lower doses of the first pharmaceutical agent or the second pharmaceutical agent (“apparent one-way synergy”), or lower doses of both pharmaceutical agents (“two-way synergy”), than would normally be required when either drug is used alone. In effect, the lower doses result in lower toxicity without reduced efficacy. A synergistic effect may additionally result in improved efficacy, including an improved avoidance or reduction of disease as compared to any single therapy.

“Synergistic effects” will also be understood to include increases in potency, bioactivity, bioaccessibility, bioavailability, or therapeutic effect (including one or more additional therapeutic effects), that are greater than the additive contributions of the components acting alone, and/or are greater than the contribution of the isolated compounds on their own.

Numerous methods known to those of skill in the art exist to determine whether there is synergy as to a particular effect, i.e., whether, when two or more components are mixed together, the effect is greater than the sum of the effects of the individual components applied alone, thereby producing “1+1>2.” Suitable methods include isobologram (or contour) analysis (Huang et al. 2019), or the equation of Loewe additivity (Loewe & Muischnek, 1926, Arch. Exp. Pathol Pharmacol. 114: 313-326). A synergistic effect also may be calculated using methods such as the Sigmoid-Emax equation (Holford & Scheiner, 1981, Clin. Pharmacokinet. 6: 429-453) and the median-effect equation (Chou & Talalay, 1984, Adv. Enzyme Regul. 22:27-55). The corresponding graphs associated with the equations referred to above are the concentration-effect curve and combination index curve, respectively. Each equation referred to above may be applied to experimental data to generate a corresponding graph to aid in assessing the effects of the drug combination.

In embodiments, a therapeutic combination comprising an I₁ agent and a CB₁ agent will be synergistic or provide synergistic effects. That is, in embodiments, a therapeutic combination will also be a “synergistic combination.” In embodiments, a therapeutic combination of the invention comprising an I₁ agonist and a CB₁ agonist will be synergistic or provide synergistic effects. In embodiments, a therapeutic combination of the invention comprising an I₁ agonist and a CB₁ antagonist will be synergistic or provide synergistic effects. In embodiments, a therapeutic combination of the invention comprising an I₁ antagonist and a CB₁ agonist will be synergistic or provide synergistic effects. In embodiments, a therapeutic combination of the invention comprising an I₁ antagonist and a CB₁ antagonist will be synergistic or provide synergistic effects.

In embodiments, a therapeutic combination comprising an I₁ agent and a 5-HT₂ agent will be synergistic or provide synergistic effects. In embodiments, a therapeutic combination of the invention comprising an I₁ agonist and a 5-HT₂ agonist will be synergistic or provide synergistic effects. In embodiments, a therapeutic combination of the invention comprising an I₁ agonist and a 5-HT₂ antagonist will be synergistic or provide synergistic effects. In embodiments, a therapeutic combination of the invention comprising an I₁ antagonist and a 5-HT₂ agonist will be synergistic or provide synergistic effects. In embodiments, a therapeutic combination of the invention comprising an I₁ antagonist and a 5-HT₂ antagonist will be synergistic or provide synergistic effects.

In embodiments, a therapeutic combination comprising an I₁ agent and a 5-HT₇ agent will be synergistic or provide synergistic effects. In embodiments, a therapeutic combination of the invention comprising an I₁ agonist and a 5-HT₇ agonist will be synergistic or provide synergistic effects. In embodiments, a therapeutic combination of the invention comprising an I₁ agonist and a 5-HT₇ antagonist will be synergistic or provide synergistic effects. In embodiments, a therapeutic combination of the invention comprising an I₁ antagonist and a 5-HT₇ agonist will be synergistic or provide synergistic effects. In embodiments, a therapeutic combination of the invention comprising an I₁ antagonist and a 5-HT₇ antagonist will be synergistic or provide synergistic effects.

In embodiments, a therapeutic combination comprising an I₁ agent, a CB₁ agent, and a 5-HT₂ agent will be synergistic or provide synergistic effects. In embodiments, a therapeutic combination of the invention comprising an I₁ agonist, a CB₁ agonist, and a 5-HT₂ agonist will be synergistic or provide synergistic effects. In embodiments, a therapeutic combination of the invention comprising an I₁ agonist, a CB₁ agonist, and a 5-HT₂ antagonist will be synergistic or provide synergistic effects. In embodiments, a therapeutic combination of the invention comprising an I₁ agonist, a CB₁ antagonist, and a 5-HT₂ agonist will be synergistic or provide synergistic effects. In embodiments, a therapeutic combination of the invention comprising an I₁ agonist, a CB₁ antagonist, and a 5-HT₂ antagonist will be synergistic or provide synergistic effects. In embodiments, a therapeutic combination of the invention comprising an I₁ antagonist, a CB₁ agonist, and a 5-HT₂ agonist will be synergistic or provide synergistic effects. In embodiments, a therapeutic combination of the invention comprising an I₁ antagonist, a CB₁ agonist, and a 5-HT₂ antagonist will be synergistic or provide synergistic effects. In embodiments, a therapeutic combination of the invention comprising an I₁ antagonist, a CB₁ antagonist, and a 5-HT₂ agonist will be synergistic or provide synergistic effects. In embodiments, a therapeutic combination of the invention comprising an I₁ antagonist, a CB₁ antagonist, and a 5-HT₂ antagonist will be synergistic or provide synergistic effects.

In embodiments, a therapeutic combination comprising an I₁ agent, a CB₁ agent, and a 5-HT₇ agent will be synergistic or provide synergistic effects. In embodiments, a therapeutic combination of the invention comprising an I₁ agonist, a CB₁ agonist, and a 5-HT₇ agonist will be synergistic or provide synergistic effects. In embodiments, a therapeutic combination of the invention comprising an I₁ agonist, a CB₁ agonist, and a 5-HT₇ antagonist will be synergistic or provide synergistic effects. In embodiments, a therapeutic combination of the invention comprising an I₁ agonist, a CB₁ antagonist, and a 5-HT₇ agonist will be synergistic or provide synergistic effects. In embodiments, a therapeutic combination of the invention comprising an I₁ agonist, a CB₁ antagonist, and a 5-HT₇ antagonist will be synergistic or provide synergistic effects. In embodiments, a therapeutic combination of the invention comprising an I₁ antagonist, a CB₁ agonist, and a 5-HT₇ agonist will be synergistic or provide synergistic effects. In embodiments, a therapeutic combination of the invention comprising an I₁ antagonist, a CB₁ agonist, and a 5-HT₇ antagonist will be synergistic or provide synergistic effects. In embodiments, a therapeutic combination of the invention comprising an I₁ antagonist, a CB₁ antagonist, and a 5-HT₇ agonist will be synergistic or provide synergistic effects. In embodiments, a therapeutic combination of the invention comprising an I₁ antagonist, a CB₁ antagonist, and a 5-HT₇ antagonist will be synergistic or provide synergistic effects.

In embodiments, a therapeutic combination comprising an I₁ agent, a 5-HT₂ agent, and a 5-HT₇ agent will be synergistic or provide synergistic effects. In embodiments, a therapeutic combination of the invention comprising an I₁ agonist, a 5-HT₂ agonist, and a 5-HT₇ agonist will be synergistic or provide synergistic effects. In embodiments, a therapeutic combination of the invention comprising an I₁ agonist, a 5-HT₂ agonist, and a 5-HT₇ antagonist will be synergistic or provide synergistic effects. In embodiments, a therapeutic combination of the invention comprising an I₁ agonist, a 5-HT₂ antagonist, and a 5-HT₇ agonist will be synergistic or provide synergistic effects. In embodiments, a therapeutic combination of the invention comprising an I₁ agonist, a 5-HT₂ antagonist, and a 5-HT₇ antagonist will be synergistic or provide synergistic effects. In embodiments, a therapeutic combination of the invention comprising an I₁ antagonist, a 5-HT₂ agonist, and a 5-HT₇ agonist will be synergistic or provide synergistic effects. In embodiments, a therapeutic combination of the invention comprising an I₁ antagonist, a 5-HT₂ agonist, and a 5-HT₇ antagonist will be synergistic or provide synergistic effects. In embodiments, a therapeutic combination of the invention comprising an I₁ antagonist, a 5-HT₂ antagonist, and a 5-HT₇ agonist will be synergistic or provide synergistic effects. In embodiments, a therapeutic combination of the invention comprising an I₁ antagonist, a 5-HT₂ antagonist, and a 5-HT₇ antagonist will be synergistic or provide synergistic effects.

In embodiments, a therapeutic combination comprising an I₁ agent, a CB₁ agent, a 5-HT₂ agent, and a 5-HT₇ agent will be synergistic or provide synergistic effects. In embodiments, a therapeutic combination of the invention comprising an I₁ agonist, a CB₁ agonist, a 5-HT₂ agonist, and a 5-HT₇ agonist will be synergistic or provide synergistic effects. In embodiments, a therapeutic combination of the invention comprising an I₁ agonist, a CB₁ agonist, a 5-HT₂ agonist, and a 5-HT₇ antagonist will be synergistic or provide synergistic effects. In embodiments, a therapeutic combination of the invention comprising an I₁ agonist, a CB₁ agonist, a 5-HT₂ antagonist, and a 5-HT₇ agonist will be synergistic or provide synergistic effects. In embodiments, a therapeutic combination of the invention comprising an I₁ agonist, a CB₁ agonist, a 5-HT₂ antagonist, and a 5-HT₇ antagonist will be synergistic or provide synergistic effects. In embodiments, a therapeutic combination of the invention comprising an I₁ agonist, a CB₁ antagonist, a 5-HT₂ agonist, and a 5-HT₇ agonist will be synergistic or provide synergistic effects. In embodiments, a therapeutic combination of the invention comprising an I₁ agonist, a CB₁ antagonist, a 5-HT₂ agonist, and a 5-HT₇ antagonist will be synergistic or provide synergistic effects. In embodiments, a therapeutic combination of the invention comprising an I₁ agonist, a CB₁ antagonist, a 5-HT₂ antagonist, and a 5-HT₇ agonist will be synergistic or provide synergistic effects. In embodiments, a therapeutic combination of the invention comprising an I₁ agonist, a CB₁ antagonist, a 5-HT₂ antagonist, and a 5-HT₇ antagonist will be synergistic or provide synergistic effects. In embodiments, a therapeutic combination of the invention comprising an I₁ antagonist, a CB₁ agonist, a 5-HT₂ agonist, and a 5-HT₇ agonist will be synergistic or provide synergistic effects. In embodiments, a therapeutic combination of the invention comprising an I₁ antagonist, a CB₁ agonist, a 5-HT₂ agonist, and a 5-HT₇ antagonist will be synergistic or provide synergistic effects. In embodiments, a therapeutic combination of the invention comprising an I₁ antagonist, a CB₁ agonist, a 5-HT₂ antagonist, and a 5-HT₇ agonist will be synergistic or provide synergistic effects. In embodiments, a therapeutic combination of the invention comprising an I₁ antagonist, a CB₁ agonist, a 5-HT₂ antagonist, and a 5-HT₇ antagonist will be synergistic or provide synergistic effects. In embodiments, a therapeutic combination of the invention comprising an I₁ antagonist, a CB₁ antagonist, a 5-HT₂ agonist, and a 5-HT₇ agonist will be synergistic or provide synergistic effects. In embodiments, a therapeutic combination of the invention comprising an I₁ antagonist, a CB₁ antagonist, a 5-HT₂ agonist, and a 5-HT₇ antagonist will be synergistic or provide synergistic effects. In embodiments, a therapeutic combination of the invention comprising an I₁ antagonist, a CB₁ antagonist, a 5-HT₂ antagonist, and a 5-HT₇ agonist will be synergistic or provide synergistic effects. In embodiments, a therapeutic combination of the invention comprising an I₁ antagonist, a CB₁ antagonist, a 5-HT₂ antagonist, and a 5-HT₇ antagonist will be synergistic or provide synergistic effects.

In embodiments, wherein MDMA is utilized as the I₁ agent, it may be combined with at least one of a CB₁, 5-HT₂, and 5-HT₇ agent, wherein the combination exhibits synergistic effects. In such embodiments, a substantially smaller dose of MDMA may be required to elicit the entactogenic state characterized by MDMA, reducing the likelihood of an adverse reaction, or reducing the likelihood of or delaying a loss of therapeutic or entactogenic effect (“loss of magic”).

### I. Exemplary Embodiments of the Invention

While many embodiments will be appreciated by one of skill through a review of the description and claims, a number of exemplary and non-limiting embodiments are disclosed. In some such exemplary embodiments, a “combination” may refer to a “therapeutic combination” wherein the two or more compounds are administered together, whether simultaneously, sequentially, or separately. In some such exemplary embodiments, a “combination” may refer to a pharmaceutical composition wherein the two or more compounds are administered together as a single formulation, along with a pharmaceutically acceptable carrier, diluent, or excipient. In some such exemplary embodiments, a “combination” may refer to a therapeutic combination or a pharmaceutical composition wherein any two or more compounds in the combination exhibit synergy or synergistic effects. In some such exemplary embodiments, a “combination” may be used in a method of the invention, such as any method as disclosed or claimed herein. In some such exemplary embodiments, a “combination” will elicit an entactogenic mindstate. Exemplary such embodiments, each of which may be used in other disclosed embodiments of the invention, now follow.

In embodiments, the combination comprises agmatine and 2-AGE. In embodiments, the combination comprises agmatine and A-834,735. In embodiments, the combination comprises agmatine and ACEA. In embodiments, the combination comprises agmatine and AZ-11713908. In embodiments, the combination comprises agmatine and AZD-1940. In embodiments, the combination comprises agmatine and CBN. In embodiments, the combination comprises agmatine and CP-47497. In embodiments, the combination comprises agmatine and CP55940. In embodiments, the combination comprises agmatine and HU-210. In embodiments, the combination comprises agmatine and JWH-007. In embodiments, the combination comprises agmatine and JWH-051. In embodiments, the combination comprises agmatine and JWH-146. In embodiments, the combination comprises agmatine and JWH-176. In embodiments, the combination comprises agmatine and JWH-200. In embodiments, the combination comprises agmatine and JWH-398. In embodiments, the combination comprises agmatine and Org 28611. In embodiments, the combination comprises agmatine and rimonabant. In embodiments, the combination comprises agmatine and THC. In embodiments, the combination comprises agmatine and WIN 55,212-2. In embodiments, the combination comprises agmatine and XLR11. In embodiments, the combination comprises agmatine and 1-methylpsilocin. In embodiments, the combination comprises agmatine and 2C-B-FLY. In embodiments, the combination comprises agmatine and 5-MeO-AMT. In embodiments, the combination comprises agmatine and AL-37350A. In embodiments, the combination comprises agmatine and CP 809101. In embodiments, the combination comprises agmatine and DALT. In embodiments, the combination comprises agmatine and DOB. In embodiments, the combination comprises agmatine and DOET In embodiments, the combination comprises agmatine and DOHx. In embodiments, the combination comprises agmatine and MEM. In embodiments, the combination comprises agmatine and CPP. In embodiments, the combination comprises agmatine and NBOH-2C-CN. In embodiments, the combination comprises agmatine and TCB-2. In embodiments, the combination comprises agmatine and WAY 161503. In embodiments, the combination comprises agmatine and 5-CT. In embodiments, the combination comprises agmatine and 5-MeO-DALT. In embodiments, the combination comprises agmatine and 5-MeO-DMT. In embodiments, the combination comprises agmatine and 5-MeO-MiPT In embodiments, the combination comprises agmatine and AGH-192. In embodiments, the combination comprises agmatine and AGH-107. In embodiments, the combination comprises agmatine and AMT. In embodiments, the combination comprises agmatine and aripiprazole. In embodiments, the combination comprises agmatine and AS-19. In embodiments, the combination comprises agmatine and E-55888. In embodiments, the combination comprises agmatine and LSD. In embodiments, the combination comprises agmatine and LP-211. In embodiments, the combination comprises agmatine and LP-44.

In embodiments, the combination comprises BDBM50091347 and 2-AGE. In embodiments, the combination comprises BDBM50091347 and A-834,735. In embodiments, the combination comprises BDBM50091347 and ACEA. In embodiments, the combination comprises BDBM50091347 and AZ-11713908. In embodiments, the combination comprises BDBM50091347 and AZD-1940. In embodiments, the combination comprises BDBM50091347 and CBN. In embodiments, the combination comprises BDBM50091347 and CP-47497. In embodiments, the combination comprises BDBM50091347 and CP55940. In embodiments, the combination comprises BDBM50091347 and HU-210. In embodiments, the combination comprises BDBM50091347 and JWH-007. In embodiments, the combination comprises BDBM50091347 and JWH-051. In embodiments, the combination comprises BDBM50091347 and JWH-146. In embodiments, the combination comprises BDBM50091347 and JWH-176. In embodiments, the combination comprises BDBM50091347 and JWH-200. In embodiments, the combination comprises BDBM50091347 and JWH-398. In embodiments, the combination comprises BDBM50091347 and Org 28611. In embodiments, the combination comprises BDBM50091347 and rimonabant. In embodiments, the combination comprises BDBM50091347 and THC. In embodiments, the combination comprises BDBM50091347 and WIN 55,212-2. In embodiments, the combination comprises BDBM50091347 and XLR11. In embodiments, the combination comprises BDBM50091347 and 1-methylpsilocin. In embodiments, the combination comprises BDBM50091347 and 2C-B-FLY. In embodiments, the combination comprises BDBM50091347 and 5-MeO-AMT. In embodiments, the combination comprises BDBM50091347 and AL-37350A. In embodiments, the combination comprises BDBM50091347 and OP 809101. In embodiments, the combination comprises BDBM50091347 and DALT. In embodiments, the combination comprises BDBM50091347 and DOB. In embodiments, the combination comprises BDBM50091347 and DOET. In embodiments, the combination comprises BDBM50091347 and DOHx. In embodiments, the combination comprises BDBM50091347 and MEM. In embodiments, the combination comprises BDBM50091347 and CPP. In embodiments, the combination comprises BDBM50091347 and NBOH-2C-CN. In embodiments, the combination comprises BDBM50091347 and TCB-2. In embodiments, the combination comprises BDBM50091347 and WAY 161503. In embodiments, the combination comprises BDBM50091347 and 5-CT. In embodiments, the combination comprises BDBM50091347 and 5-MeO-DALT. In embodiments, the combination comprises BDBM50091347 and 5-MeO-DMT In embodiments, the combination comprises BDBM50091347 and 5-MeO-MiPT. In embodiments, the combination comprises BDBM50091347 and AGH-192. In embodiments, the combination comprises BDBM50091347 and AGH-107. In embodiments, the combination comprises BDBM50091347 and AMT. In embodiments, the combination comprises BDBM50091347 and aripiprazole. In embodiments, the combination comprises BDBM50091347 and AS-19. In embodiments, the combination comprises BDBM50091347 and E-55888. In embodiments, the combination comprises BDBM50091347 and LSD. In embodiments, the combination comprises BDBM50091347 and LP-211. In embodiments, the combination comprises BDBM50091347 and LP-44.

In embodiments, the combination comprises clonidine and 2-AGE. In embodiments, the combination comprises clonidine and A-834,735. In embodiments, the combination comprises clonidine and ACEA. In embodiments, the combination comprises clonidine and AZ-11713908. In embodiments, the combination comprises clonidine and AZD-1940. In embodiments, the combination comprises clonidine and CBN. In embodiments, the combination comprises clonidine and CP-47497. In embodiments, the combination comprises clonidine and CP55940. In embodiments, the combination comprises clonidine and HU-210. In embodiments, the combination comprises clonidine and JWH-007. In embodiments, the combination comprises clonidine and JWH-051. In embodiments, the combination comprises clonidine and JWH-146. In embodiments, the combination comprises clonidine and JWH-176. In embodiments, the combination comprises clonidine and JWH-200. In embodiments, the combination comprises clonidine and JWH-398. In embodiments, the combination comprises clonidine and Org 28611. In embodiments, the combination comprises clonidine and rimonabant. In embodiments, the combination comprises clonidine and THC. In embodiments, the combination comprises clonidine and WIN 55,212-2. In embodiments, the combination comprises clonidine and XLR11. In embodiments, the combination comprises clonidine and 1-methylpsilocin. In embodiments, the combination comprises clonidine and 2C-B-FLY. In embodiments, the combination comprises clonidine and 5-MeO-AMT. In embodiments, the combination comprises clonidine and AL-37350A. In embodiments, the combination comprises clonidine and CP 809101. In embodiments, the combination comprises clonidine and DALT. In embodiments, the combination comprises clonidine and DOB. In embodiments, the combination comprises clonidine and DOET In embodiments, the combination comprises clonidine and DOHx. In embodiments, the combination comprises clonidine and MEM. In embodiments, the combination comprises clonidine and CPP. In embodiments, the combination comprises clonidine and NBOH-2C-CN. In embodiments, the combination comprises clonidine and TCB-2. In embodiments, the combination comprises clonidine and WAY 161503. In embodiments, the combination comprises clonidine and 5-CT. In embodiments, the combination comprises clonidine and 5-MeO-DALT. In embodiments, the combination comprises clonidine and 5-MeO-DMT. In embodiments, the combination comprises clonidine and 5-MeO-MiPT In embodiments, the combination comprises clonidine and AGH-192. In embodiments, the combination comprises clonidine and AGH-107. In embodiments, the combination comprises clonidine and AMT. In embodiments, the combination comprises clonidine and aripiprazole. In embodiments, the combination comprises clonidine and AS-19. In embodiments, the combination comprises clonidine and E-55888. In embodiments, the combination comprises clonidine and LSD. In embodiments, the combination comprises clonidine and LP-211. In embodiments, the combination comprises clonidine and LP-44.

In embodiments, the combination comprises guanfacine and 2-AGE. In embodiments, the combination comprises guanfacine and A-834,735. In embodiments, the combination comprises guanfacine and ACEA. In embodiments, the combination comprises guanfacine and AZ-11713908. In embodiments, the combination comprises guanfacine and AZD-1940. In embodiments, the combination comprises guanfacine and CBN. In embodiments, the combination comprises guanfacine and CP-47497. In embodiments, the combination comprises guanfacine and CP55940. In embodiments, the combination comprises guanfacine and HU-210. In embodiments, the combination comprises guanfacine and JWH-007. In embodiments, the combination comprises guanfacine and JWH-051. In embodiments, the combination comprises guanfacine and JWH-146. In embodiments, the combination comprises guanfacine and JWH-176. In embodiments, the combination comprises guanfacine and JWH-200. In embodiments, the combination comprises guanfacine and JWH-398. In embodiments, the combination comprises guanfacine and Org 28611. In embodiments, the combination comprises guanfacine and rimonabant. In embodiments, the combination comprises guanfacine and THC. In embodiments, the combination comprises guanfacine and WIN 55,212-2. In embodiments, the combination comprises guanfacine and XLR11. In embodiments, the combination comprises guanfacine and 1-methylpsilocin. In embodiments, the combination comprises guanfacine and 2C-B-FLY. In embodiments, the combination comprises guanfacine and 5-MeO-AMT. In embodiments, the combination comprises guanfacine and AL-37350A. In embodiments, the combination comprises guanfacine and CP 809101. In embodiments, the combination comprises guanfacine and DALT. In embodiments, the combination comprises guanfacine and DOB. In embodiments, the combination comprises guanfacine and DOET. In embodiments, the combination comprises guanfacine and DOHx. In embodiments, the combination comprises guanfacine and MEM. In embodiments, the combination comprises guanfacine and CPP. In embodiments, the combination comprises guanfacine and NBOH-2C-CN. In embodiments, the combination comprises guanfacine and TCB-2. In embodiments, the combination comprises guanfacine and WAY 161503. In embodiments, the combination comprises guanfacine and 5-CT. In embodiments, the combination comprises guanfacine and 5-MeO-DALT. In embodiments, the combination comprises guanfacine and 5-MeO-DMT. In embodiments, the combination comprises guanfacine and 5-MeO-MiPT. In embodiments, the combination comprises guanfacine and AGH-192. In embodiments, the combination comprises guanfacine and AGH-107. In embodiments, the combination comprises guanfacine and AMT. In embodiments, the combination comprises guanfacine and aripiprazole. In embodiments, the combination comprises guanfacine and AS-19. In embodiments, the combination comprises guanfacine and E-55888. In embodiments, the combination comprises guanfacine and LSD. In embodiments, the combination comprises guanfacine and LP-211. In embodiments, the combination comprises guanfacine and LP-44.

In embodiments, the combination comprises mCPP and 2-AGE. In embodiments, the combination comprises mCPP and A-834,735. In embodiments, the combination comprises mCPP and ACEA. In embodiments, the combination comprises mCPP and AZ-11713908. In embodiments, the combination comprises mCPP and AZD-1940. In embodiments, the combination comprises mCPP and CBN. In embodiments, the combination comprises mCPP and CP-47497. In embodiments, the combination comprises mCPP and CP55940. In embodiments, the combination comprises mCPP and HU-210. In embodiments, the combination comprises mCPP and JWH-007. In embodiments, the combination comprises mCPP and JWH-051. In embodiments, the combination comprises mCPP and JWH-146. In embodiments, the combination comprises mCPP and JWH-176. In embodiments, the combination comprises mCPP and JWH-200. In embodiments, the combination comprises mCPP and JWH-398. In embodiments, the combination comprises mCPP and Org 28611. In embodiments, the combination comprises mCPP and rimonabant. In embodiments, the combination comprises mCPP and THC. In embodiments, the combination comprises mCPP and WIN 55,212-2. In embodiments, the combination comprises mCPP and XLR11. In embodiments, the combination comprises mCPP and 1-methylpsilocin. In embodiments, the combination comprises mCPP and 2C-B-FLY. In embodiments, the combination comprises mCPP and 5-MeO-AMT. In embodiments, the combination comprises mCPP and AL-37350A. In embodiments, the combination comprises mCPP and CP 809101. In embodiments, the combination comprises mCPP and DALT. In embodiments, the combination comprises mCPP and DOB. In embodiments, the combination comprises mCPP and DOET In embodiments, the combination comprises mCPP and DOHx. In embodiments, the combination comprises mCPP and MEM. In embodiments, the combination comprises mCPP and CPP. In embodiments, the combination comprises mCPP and NBOH-2C-CN. In embodiments, the combination comprises mCPP and TCB-2. In embodiments, the combination comprises mCPP and WAY 161503. In embodiments, the combination comprises mCPP and 5-CT. In embodiments, the combination comprises mCPP and 5-MeO-DALT. In embodiments, the combination comprises mCPP and 5-MeO-DMT. In embodiments, the combination comprises mCPP and 5-MeO-MiPT. In embodiments, the combination comprises mCPP and AGH-192. In embodiments, the combination comprises mCPP and AGH-107. In embodiments, the combination comprises mCPP and AMT. In embodiments, the combination comprises mCPP and aripiprazole. In embodiments, the combination comprises mCPP and AS-19. In embodiments, the combination comprises mCPP and E-55888. In embodiments, the combination comprises mCPP and LSD. In embodiments, the combination comprises mCPP and LP-211. In embodiments, the combination comprises mCPP and LP-44.

In embodiments, the combination comprises MDMA and 2-AGE. In embodiments, the combination comprises MDMA and A-834,735. In embodiments, the combination comprises MDMA and ACEA. In embodiments, the combination comprises MDMA and AZ-11713908. In embodiments, the combination comprises MDMA and AZD-1940. In embodiments, the combination comprises MDMA and CBN. In embodiments, the combination comprises MDMA and CP-47497. In embodiments, the combination comprises MDMA and CP55940. In embodiments, the combination comprises MDMA and HU-210. In embodiments, the combination comprises MDMA and JWH-007. In embodiments, the combination comprises MDMA and JWH-051. In embodiments, the combination comprises MDMA and JWH-146. In embodiments, the combination comprises MDMA and JWH-176. In embodiments, the combination comprises MDMA and JWH-200. In embodiments, the combination comprises MDMA and JWH-398. In embodiments, the combination comprises MDMA and Org 28611. In embodiments, the combination comprises MDMA and rimonabant. In embodiments, the combination comprises MDMA and THC. In embodiments, the combination comprises MDMA and WIN 55,212-2. In embodiments, the combination comprises MDMA and XLR11. In embodiments, the combination comprises MDMA and 1-methylpsilocin. In embodiments, the combination comprises MDMA and 2C-B-FLY. In embodiments, the combination comprises MDMA and 5-MeO-AMT. In embodiments, the combination comprises MDMA and AL-37350A. In embodiments, the combination comprises MDMA and CP 809101. In embodiments, the combination comprises MDMA and DALT. In embodiments, the combination comprises MDMA and DOB. In embodiments, the combination comprises MDMA and DOET. In embodiments, the combination comprises MDMA and DOHx. In embodiments, the combination comprises MDMA and MEM. In embodiments, the combination comprises MDMA and CPP. In embodiments, the combination comprises MDMA and NBOH-2C-CN. In embodiments, the combination comprises MDMA and TCB-2. In embodiments, the combination comprises MDMA and WAY 161503. In embodiments, the combination comprises MDMA and 5-CT. In embodiments, the combination comprises MDMA and 5-MeO-DALT. In embodiments, the combination comprises MDMA and 5-MeO-DMT In embodiments, the combination comprises MDMA and 5-MeO-MiPT. In embodiments, the combination comprises MDMA and AGH-192. In embodiments, the combination comprises MDMA and AGH-107. In embodiments, the combination comprises MDMA and AMT. In embodiments, the combination comprises MDMA and aripiprazole. In embodiments, the combination comprises MDMA and AS-19. In embodiments, the combination comprises MDMA and E-55888. In embodiments, the combination comprises MDMA and LSD. In embodiments, the combination comprises MDMA and LP-211. In embodiments, the combination comprises MDMA and LP-44.

In embodiments, the combination comprises memantine and 2-AGE. In embodiments, the combination comprises memantine and A-834,735. In embodiments, the combination comprises memantine and ACEA. In embodiments, the combination comprises memantine and AZ-11713908. In embodiments, the combination comprises memantine and AZD-1940. In embodiments, the combination comprises memantine and CBN. In embodiments, the combination comprises memantine and CP-47497. In embodiments, the combination comprises memantine and CP55940. In embodiments, the combination comprises memantine and HU-210. In embodiments, the combination comprises memantine and JWH-007. In embodiments, the combination comprises memantine and JWH-051. In embodiments, the combination comprises memantine and JWH-146. In embodiments, the combination comprises memantine and JWH-176. In embodiments, the combination comprises memantine and JWH-200. In embodiments, the combination comprises memantine and JWH-398. In embodiments, the combination comprises memantine and Org 28611. In embodiments, the combination comprises memantine and rimonabant. In embodiments, the combination comprises memantine and THC. In embodiments, the combination comprises memantine and WIN 55,212-2. In embodiments, the combination comprises memantine and XLR11. In embodiments, the combination comprises memantine and 1-methylpsilocin. In embodiments, the combination comprises memantine and 2C-B-FLY. In embodiments, the combination comprises memantine and 5-MeO-AMT. In embodiments, the combination comprises memantine and AL-37350A. In embodiments, the combination comprises memantine and CP 809101. In embodiments, the combination comprises memantine and DALT. In embodiments, the combination comprises memantine and DOB. In embodiments, the combination comprises memantine and DOET In embodiments, the combination comprises memantine and DOHx. In embodiments, the combination comprises memantine and MEM. In embodiments, the combination comprises memantine and CPP. In embodiments, the combination comprises memantine and NBOH-2C-CN. In embodiments, the combination comprises memantine and TCB-2. In embodiments, the combination comprises memantine and WAY 161503. In embodiments, the combination comprises memantine and 5-CT. In embodiments, the combination comprises memantine and 5-MeO-DALT. In embodiments, the combination comprises memantine and 5-MeO-DMT. In embodiments, the combination comprises memantine and 5-MeO-MiPT. In embodiments, the combination comprises memantine and AGH-192. In embodiments, the combination comprises memantine and AGH-107. In embodiments, the combination comprises memantine and AMT. In embodiments, the combination comprises memantine and aripiprazole. In embodiments, the combination comprises memantine and AS-19. In embodiments, the combination comprises memantine and E-55888. In embodiments, the combination comprises memantine and LSD. In embodiments, the combination comprises memantine and LP-211. In embodiments, the combination comprises memantine and LP-44.

In embodiments, the combination comprises moxonidine and 2-AGE. In embodiments, the combination comprises moxonidine and A-834,735. In embodiments, the combination comprises moxonidine and ACEA. In embodiments, the combination comprises moxonidine and AZ-11713908. In embodiments, the combination comprises moxonidine and AZD-1940. In embodiments, the combination comprises moxonidine and CBN. In embodiments, the combination comprises moxonidine and CP-47497. In embodiments, the combination comprises moxonidine and CP55940. In embodiments, the combination comprises moxonidine and HU-210. In embodiments, the combination comprises moxonidine and JWH-007. In embodiments, the combination comprises moxonidine and JWH-051. In embodiments, the combination comprises moxonidine and JWH-146. In embodiments, the combination comprises moxonidine and JWH-176. In embodiments, the combination comprises moxonidine and JWH-200. In embodiments, the combination comprises moxonidine and JWH-398. In embodiments, the combination comprises moxonidine and Org 28611. In embodiments, the combination comprises moxonidine and rimonabant. In embodiments, the combination comprises moxonidine and THC. In embodiments, the combination comprises moxonidine and WIN 55,212-2. In embodiments, the combination comprises moxonidine and XLR11. In embodiments, the combination comprises moxonidine and 1-methylpsilocin. In embodiments, the combination comprises moxonidine and 2C-B-FLY. In embodiments, the combination comprises moxonidine and 5-MeO-AMT. In embodiments, the combination comprises moxonidine and AL-37350A. In embodiments, the combination comprises moxonidine and CP 809101. In embodiments, the combination comprises moxonidine and DALT. In embodiments, the combination comprises moxonidine and DOB. In embodiments, the combination comprises moxonidine and DOET. In embodiments, the combination comprises moxonidine and DOHx. In embodiments, the combination comprises moxonidine and MEM. In embodiments, the combination comprises moxonidine and CPP. In embodiments, the combination comprises moxonidine and NBOH-2C-CN. In embodiments, the combination comprises moxonidine and TCB-2. In embodiments, the combination comprises moxonidine and WAY 161503. In embodiments, the combination comprises moxonidine and 5-CT. In embodiments, the combination comprises moxonidine and 5-MeO-DALT. In embodiments, the combination comprises moxonidine and 5-MeO-DMT. In embodiments, the combination comprises moxonidine and 5-MeO-MiPT In embodiments, the combination comprises moxonidine and AGH-192. In embodiments, the combination comprises moxonidine and AGH-107. In embodiments, the combination comprises moxonidine and AMT. In embodiments, the combination comprises moxonidine and aripiprazole. In embodiments, the combination comprises moxonidine and AS-19. In embodiments, the combination comprises moxonidine and E-55888. In embodiments, the combination comprises moxonidine and LSD. In embodiments, the combination comprises moxonidine and LP-211. In embodiments, the combination comprises moxonidine and LP-44.

In embodiments, the combination comprises naphazoline and 2-AGE. In embodiments, the combination comprises naphazoline and A-834,735. In embodiments, the combination comprises naphazoline and ACEA. In embodiments, the combination comprises naphazoline and AZ-11713908. In embodiments, the combination comprises naphazoline and AZD-1940. In embodiments, the combination comprises naphazoline and CBN. In embodiments, the combination comprises naphazoline and CP-47497. In embodiments, the combination comprises naphazoline and CP55940. In embodiments, the combination comprises naphazoline and HU-210. In embodiments, the combination comprises naphazoline and JWH-007. In embodiments, the combination comprises naphazoline and JWH-051. In embodiments, the combination comprises naphazoline and JWH-146. In embodiments, the combination comprises naphazoline and JWH-176. In embodiments, the combination comprises naphazoline and JWH-200. In embodiments, the combination comprises naphazoline and JWH-398. In embodiments, the combination comprises naphazoline and Org 28611. In embodiments, the combination comprises naphazoline and rimonabant. In embodiments, the combination comprises naphazoline and THC. In embodiments, the combination comprises naphazoline and WIN 55,212-2. In embodiments, the combination comprises naphazoline and XLR11. In embodiments, the combination comprises naphazoline and 1-methylpsilocin. In embodiments, the combination comprises naphazoline and 2C-B-FLY. In embodiments, the combination comprises naphazoline and 5-MeO-AMT. In embodiments, the combination comprises naphazoline and AL-37350A. In embodiments, the combination comprises naphazoline and CP 809101. In embodiments, the combination comprises naphazoline and DALT. In embodiments, the combination comprises naphazoline and DOB. In embodiments, the combination comprises naphazoline and DOET. In embodiments, the combination comprises naphazoline and DOHx. In embodiments, the combination comprises naphazoline and MEM. In embodiments, the combination comprises naphazoline and CPP. In embodiments, the combination comprises naphazoline and NBOH-2C-CN. In embodiments, the combination comprises naphazoline and TCB-2. In embodiments, the combination comprises naphazoline and WAY 161503. In embodiments, the combination comprises naphazoline and 5-CT. In embodiments, the combination comprises naphazoline and 5-MeO-DALT. In embodiments, the combination comprises naphazoline and 5-MeO-DMT. In embodiments, the combination comprises naphazoline and 5-MeO-MiPT. In embodiments, the combination comprises naphazoline and AGH-192. In embodiments, the combination comprises naphazoline and AGH-107. In embodiments, the combination comprises naphazoline and AMT. In embodiments, the combination comprises naphazoline and aripiprazole. In embodiments, the combination comprises naphazoline and AS-19. In embodiments, the combination comprises naphazoline and E-55888. In embodiments, the combination comprises naphazoline and LSD. In embodiments, the combination comprises naphazoline and LP-211. In embodiments, the combination comprises naphazoline and LP-44.

In embodiments, the combination comprises oxymetazoline and 2-AGE. In embodiments, the combination comprises oxymetazoline and A-834,735. In embodiments, the combination comprises oxymetazoline and ACEA. In embodiments, the combination comprises oxymetazoline and AZ-11713908. In embodiments, the combination comprises oxymetazoline and AZD-1940. In embodiments, the combination comprises oxymetazoline and CBN. In embodiments, the combination comprises oxymetazoline and CP-47497. In embodiments, the combination comprises oxymetazoline and CP55940. In embodiments, the combination comprises oxymetazoline and HU-210. In embodiments, the combination comprises oxymetazoline and JWH-007. In embodiments, the combination comprises oxymetazoline and JWH-051. In embodiments, the combination comprises oxymetazoline and JWH-146. In embodiments, the combination comprises oxymetazoline and JWH-176. In embodiments, the combination comprises oxymetazoline and JWH-200. In embodiments, the combination comprises oxymetazoline and JWH-398. In embodiments, the combination comprises oxymetazoline and Org 28611. In embodiments, the combination comprises oxymetazoline and rimonabant. In embodiments, the combination comprises oxymetazoline and THC. In embodiments, the combination comprises oxymetazoline and WIN 55,212-2. In embodiments, the combination comprises oxymetazoline and XLR11. In embodiments, the combination comprises oxymetazoline and 1-methylpsilocin. In embodiments, the combination comprises oxymetazoline and 2C-B-FLY. In embodiments, the combination comprises oxymetazoline and 5-MeO-AMT. In embodiments, the combination comprises oxymetazoline and AL-37350A. In embodiments, the combination comprises oxymetazoline and CP 809101. In embodiments, the combination comprises oxymetazoline and DALT. In embodiments, the combination comprises oxymetazoline and DOB. In embodiments, the combination comprises oxymetazoline and DOET In embodiments, the combination comprises oxymetazoline and DOHx. In embodiments, the combination comprises oxymetazoline and MEM. In embodiments, the combination comprises oxymetazoline and CPP. In embodiments, the combination comprises oxymetazoline and NBOH-2C-CN. In embodiments, the combination comprises oxymetazoline and TCB-2. In embodiments, the combination comprises oxymetazoline and WAY 161503. In embodiments, the combination comprises oxymetazoline and 5-CT. In embodiments, the combination comprises oxymetazoline and 5-MeO-DALT. In embodiments, the combination comprises oxymetazoline and 5-MeO-DMT. In embodiments, the combination comprises oxymetazoline and 5-MeO-MiPT In embodiments, the combination comprises oxymetazoline and AGH-192. In embodiments, the combination comprises oxymetazoline and AGH-107. In embodiments, the combination comprises oxymetazoline and AMT. In embodiments, the combination comprises oxymetazoline and aripiprazole. In embodiments, the combination comprises oxymetazoline and AS-19. In embodiments, the combination comprises oxymetazoline and E-55888. In embodiments, the combination comprises oxymetazoline and LSD. In embodiments, the combination comprises oxymetazoline and LP-211. In embodiments, the combination comprises oxymetazoline and LP-44.

In embodiments, the combination comprises rilmenidine and 2-AGE. In embodiments, the combination comprises rilmenidine and A-834,735. In embodiments, the combination comprises rilmenidine and ACEA. In embodiments, the combination comprises rilmenidine and AZ-11713908. In embodiments, the combination comprises rilmenidine and AZD-1940. In embodiments, the combination comprises rilmenidine and CBN. In embodiments, the combination comprises rilmenidine and CP-47497. In embodiments, the combination comprises rilmenidine and CP55940. In embodiments, the combination comprises rilmenidine and HU-210. In embodiments, the combination comprises rilmenidine and JWH-007. In embodiments, the combination comprises rilmenidine and JWH-051. In embodiments, the combination comprises rilmenidine and JWH-146. In embodiments, the combination comprises rilmenidine and JWH-176. In embodiments, the combination comprises rilmenidine and JWH-200. In embodiments, the combination comprises rilmenidine and JWH-398. In embodiments, the combination comprises rilmenidine and Org 28611. In embodiments, the combination comprises rilmenidine and rimonabant. In embodiments, the combination comprises rilmenidine and THC. In embodiments, the combination comprises rilmenidine and WIN 55,212-2. In embodiments, the combination comprises rilmenidine and XLR11. In embodiments, the combination comprises rilmenidine and 1-methylpsilocin. In embodiments, the combination comprises rilmenidine and 2C-B-FLY. In embodiments, the combination comprises rilmenidine and 5-MeO-AMT. In embodiments, the combination comprises rilmenidine and AL-37350A. In embodiments, the combination comprises rilmenidine and CP 809101. In embodiments, the combination comprises rilmenidine and DALT. In embodiments, the combination comprises rilmenidine and DOB. In embodiments, the combination comprises rilmenidine and DOET. In embodiments, the combination comprises rilmenidine and DOHx. In embodiments, the combination comprises rilmenidine and MEM. In embodiments, the combination comprises rilmenidine and CPP. In embodiments, the combination comprises rilmenidine and NBOH-2C-CN. In embodiments, the combination comprises rilmenidine and TCB-2. In embodiments, the combination comprises rilmenidine and WAY 161503. In embodiments, the combination comprises rilmenidine and 5-CT. In embodiments, the combination comprises rilmenidine and 5-MeO-DALT. In embodiments, the combination comprises rilmenidine and 5-MeO-DMT. In embodiments, the combination comprises rilmenidine and 5-MeO-MiPT In embodiments, the combination comprises rilmenidine and AGH-192. In embodiments, the combination comprises rilmenidine and AGH-107. In embodiments, the combination comprises rilmenidine and AMT. In embodiments, the combination comprises rilmenidine and aripiprazole. In embodiments, the combination comprises rilmenidine and AS-19. In embodiments, the combination comprises rilmenidine and E-55888. In embodiments, the combination comprises rilmenidine and LSD. In embodiments, the combination comprises rilmenidine and LP-211. In embodiments, the combination comprises rilmenidine and LP-44.

In embodiments, the combination comprises tetryzoline and 2-AGE. In embodiments, the combination comprises tetryzoline and A-834,735. In embodiments, the combination comprises tetryzoline and ACEA. In embodiments, the combination comprises tetryzoline and AZ-11713908. In embodiments, the combination comprises tetryzoline and AZD-1940. In embodiments, the combination comprises tetryzoline and CBN. In embodiments, the combination comprises tetryzoline and CP-47497. In embodiments, the combination comprises tetryzoline and CP55940. In embodiments, the combination comprises tetryzoline and HU-210. In embodiments, the combination comprises tetryzoline and JWH-007. In embodiments, the combination comprises tetryzoline and JWH-051. In embodiments, the combination comprises tetryzoline and JWH-146. In embodiments, the combination comprises tetryzoline and JWH-176. In embodiments, the combination comprises tetryzoline and JWH-200. In embodiments, the combination comprises tetryzoline and JWH-398. In embodiments, the combination comprises tetryzoline and Org 28611. In embodiments, the combination comprises tetryzoline and rimonabant. In embodiments, the combination comprises tetryzoline and THC. In embodiments, the combination comprises tetryzoline and WIN 55,212-2. In embodiments, the combination comprises tetryzoline and XLR11. In embodiments, the combination comprises tetryzoline and 1-methylpsilocin. In embodiments, the combination comprises tetryzoline and 2C-B-FLY. In embodiments, the combination comprises tetryzoline and 5-MeO-AMT. In embodiments, the combination comprises tetryzoline and AL-37350A. In embodiments, the combination comprises tetryzoline and CP 809101. In embodiments, the combination comprises tetryzoline and DALT. In embodiments, the combination comprises tetryzoline and DOB. In embodiments, the combination comprises tetryzoline and DOET In embodiments, the combination comprises tetryzoline and DOHx. In embodiments, the combination comprises tetryzoline and MEM. In embodiments, the combination comprises tetryzoline and CPP. In embodiments, the combination comprises tetryzoline and NBOH-2C-CN. In embodiments, the combination comprises tetryzoline and TCB-2. In embodiments, the combination comprises tetryzoline and WAY 161503. In embodiments, the combination comprises tetryzoline and 5-CT. In embodiments, the combination comprises tetryzoline and 5-MeO-DALT. In embodiments, the combination comprises tetryzoline and 5-MeO-DMT. In embodiments, the combination comprises tetryzoline and 5-MeO-MiPT In embodiments, the combination comprises tetryzoline and AGH-192. In embodiments, the combination comprises tetryzoline and AGH-107. In embodiments, the combination comprises tetryzoline and AMT. In embodiments, the combination comprises tetryzoline and aripiprazole. In embodiments, the combination comprises tetryzoline and AS-19. In embodiments, the combination comprises tetryzoline and E-55888. In embodiments, the combination comprises tetryzoline and LSD. In embodiments, the combination comprises tetryzoline and LP-211. In embodiments, the combination comprises tetryzoline and LP-44.

In embodiments, the combination comprises tizanidine and 2-AGE. In embodiments, the combination comprises tizanidine and A-834,735. In embodiments, the combination comprises tizanidine and ACEA. In embodiments, the combination comprises tizanidine and AZ-11713908. In embodiments, the combination comprises tizanidine and AZD-1940. In embodiments, the combination comprises tizanidine and CBN. In embodiments, the combination comprises tizanidine and CP-47497. In embodiments, the combination comprises tizanidine and CP55940. In embodiments, the combination comprises tizanidine and HU-210. In embodiments, the combination comprises tizanidine and JWH-007. In embodiments, the combination comprises tizanidine and JWH-051. In embodiments, the combination comprises tizanidine and JWH-146. In embodiments, the combination comprises tizanidine and JWH-176. In embodiments, the combination comprises tizanidine and JWH-200. In embodiments, the combination comprises tizanidine and JWH-398. In embodiments, the combination comprises tizanidine and Org 28611. In embodiments, the combination comprises tizanidine and rimonabant. In embodiments, the combination comprises tizanidine and THC. In embodiments, the combination comprises tizanidine and WIN 55,212-2. In embodiments, the combination comprises tizanidine and XLR11. In embodiments, the combination comprises tizanidine and 1-methylpsilocin. In embodiments, the combination comprises tizanidine and 2C-B-FLY. In embodiments, the combination comprises tizanidine and 5-MeO-AMT. In embodiments, the combination comprises tizanidine and AL-37350A. In embodiments, the combination comprises tizanidine and CP 809101. In embodiments, the combination comprises tizanidine and DALT. In embodiments, the combination comprises tizanidine and DOB. In embodiments, the combination comprises tizanidine and DOET In embodiments, the combination comprises tizanidine and DOHx. In embodiments, the combination comprises tizanidine and MEM. In embodiments, the combination comprises tizanidine and CPP. In embodiments, the combination comprises tizanidine and NBOH-2C-CN. In embodiments, the combination comprises tizanidine and TCB-2. In embodiments, the combination comprises tizanidine and WAY 161503. In embodiments, the combination comprises tizanidine and 5-CT. In embodiments, the combination comprises tizanidine and 5-MeO-DALT. In embodiments, the combination comprises tizanidine and 5-MeO-DMT In embodiments, the combination comprises tizanidine and 5-MeO-MiPT. In embodiments, the combination comprises tizanidine and AGH-192. In embodiments, the combination comprises tizanidine and AGH-107. In embodiments, the combination comprises tizanidine and AMT. In embodiments, the combination comprises tizanidine and aripiprazole. In embodiments, the combination comprises tizanidine and AS-19. In embodiments, the combination comprises tizanidine and E-55888. In embodiments, the combination comprises tizanidine and LSD. In embodiments, the combination comprises tizanidine and LP-211. In embodiments, the combination comprises tizanidine and LP-44.

In embodiments, the combination comprises tolonidine and 2-AGE. In embodiments, the combination comprises tolonidine and A-834,735. In embodiments, the combination comprises tolonidine and ACEA. In embodiments, the combination comprises tolonidine and AZ-11713908. In embodiments, the combination comprises tolonidine and AZD-1940. In embodiments, the combination comprises tolonidine and CBN. In embodiments, the combination comprises tolonidine and CP-47497. In embodiments, the combination comprises tolonidine and CP55940. In embodiments, the combination comprises tolonidine and HU-210. In embodiments, the combination comprises tolonidine and JWH-007. In embodiments, the combination comprises tolonidine and JWH-051. In embodiments, the combination comprises tolonidine and JWH-146. In embodiments, the combination comprises tolonidine and JWH-176. In embodiments, the combination comprises tolonidine and JWH-200. In embodiments, the combination comprises tolonidine and JWH-398. In embodiments, the combination comprises tolonidine and Org 28611. In embodiments, the combination comprises tolonidine and rimonabant. In embodiments, the combination comprises tolonidine and THC. In embodiments, the combination comprises tolonidine and WIN 55,212-2. In embodiments, the combination comprises tolonidine and XLR11. In embodiments, the combination comprises tolonidine and 1-methylpsilocin. In embodiments, the combination comprises tolonidine and 2C-B-FLY. In embodiments, the combination comprises tolonidine and 5-MeO-AMT. In embodiments, the combination comprises tolonidine and AL-37350A. In embodiments, the combination comprises tolonidine and CP 809101. In embodiments, the combination comprises tolonidine and DALT. In embodiments, the combination comprises tolonidine and DOB. In embodiments, the combination comprises tolonidine and DOET In embodiments, the combination comprises tolonidine and DOHx. In embodiments, the combination comprises tolonidine and MEM. In embodiments, the combination comprises tolonidine and CPP. In embodiments, the combination comprises tolonidine and NBOH-2C-CN. In embodiments, the combination comprises tolonidine and TCB-2. In embodiments, the combination comprises tolonidine and WAY 161503. In embodiments, the combination comprises tolonidine and 5-CT. In embodiments, the combination comprises tolonidine and 5-MeO-DALT. In embodiments, the combination comprises tolonidine and 5-MeO-DMT. In embodiments, the combination comprises tolonidine and 5-MeO-MiPT. In embodiments, the combination comprises tolonidine and AGH-192. In embodiments, the combination comprises tolonidine and AGH-107. In embodiments, the combination comprises tolonidine and AMT. In embodiments, the combination comprises tolonidine and aripiprazole. In embodiments, the combination comprises tolonidine and AS-19. In embodiments, the combination comprises tolonidine and E-55888. In embodiments, the combination comprises tolonidine and LSD. In embodiments, the combination comprises tolonidine and LP-211. In embodiments, the combination comprises tolonidine and LP-44.

In embodiments, the combination comprises agmatine, a CB₁ agent, and 1-methylpsilocin. In embodiments, the combination comprises agmatine, a CB₁ agent, and 2C-B-FLY. In embodiments, the combination comprises agmatine, a CB₁ agent, and 5-MeO-AMT. In embodiments, the combination comprises agmatine, a CB₁ agent, and AL-37350A. In embodiments, the combination comprises agmatine, a CB₁ agent, and CP 809101. In embodiments, the combination comprises agmatine, a CB₁ agent, and DALT. In embodiments, the combination comprises agmatine, a CB₁ agent, and DOB. In embodiments, the combination comprises agmatine, a CB₁ agent, and DOET In embodiments, the combination comprises agmatine, a CB₁ agent, and DOHx. In embodiments, the combination comprises agmatine, a CB₁ agent, and MEM. In embodiments, the combination comprises agmatine, a CB₁ agent, and CPP. In embodiments, the combination comprises agmatine, a CB₁ agent, and NBOH-2C-CN. In embodiments, the combination comprises agmatine, a CB₁ agent, and TCB-2. In embodiments, the combination comprises agmatine, a CB₁ agent, and WAY 161503. In embodiments, the combination comprises agmatine, a CB₁ agent, and 5-CT. In embodiments, the combination comprises agmatine, a CB₁ agent, and 5-MeO-DALT. In embodiments, the combination comprises agmatine, a CB₁ agent, and 5-MeO-DMT. In embodiments, the combination comprises agmatine, a CB₁ agent, and 5-MeO-MiPT In embodiments, the combination comprises agmatine, a CB₁ agent, and AGH-192. In embodiments, the combination comprises agmatine, a CB₁ agent, and AGH-107. In embodiments, the combination comprises agmatine, a CB₁ agent, and AMT. In embodiments, the combination comprises agmatine, a CB₁ agent, and aripiprazole. In embodiments, the combination comprises agmatine, a CB₁ agent, and AS-19. In embodiments, the combination comprises agmatine, a CB₁ agent, and E-55888. In embodiments, the combination comprises agmatine, a CB₁ agent, and LSD. In embodiments, the combination comprises agmatine, a CB₁ agent, and LP-211. In embodiments, the combination comprises agmatine, a CB₁ agent, and LP-44.

In embodiments, the combination comprises BDBM50091347, a CB₁ agent, and 1-methylpsilocin. In embodiments, the combination comprises BDBM50091347, a CB₁ agent, and 2C-B-FLY. In embodiments, the combination comprises BDBM50091347, a CB₁ agent, and 5-MeO-AMT. In embodiments, the combination comprises BDBM50091347, a CB₁ agent, and AL-37350A. In embodiments, the combination comprises BDBM50091347, a CB₁ agent, and CP 809101. In embodiments, the combination comprises BDBM50091347, a CB₁ agent, and DALT. In embodiments, the combination comprises BDBM50091347, a CB₁ agent, and DOB. In embodiments, the combination comprises BDBM50091347, a CB₁ agent, and DOET. In embodiments, the combination comprises BDBM50091347, a CB₁ agent, and DOHx. In embodiments, the combination comprises BDBM50091347, a CB₁ agent, and MEM. In embodiments, the combination comprises BDBM50091347, a CB₁ agent, and CPP. In embodiments, the combination comprises BDBM50091347, a CB₁ agent, and NBOH-2C-CN. In embodiments, the combination comprises BDBM50091347, a CB₁ agent, and TCB-2. In embodiments, the combination comprises BDBM50091347, a CB₁ agent, and WAY 161503. In embodiments, the combination comprises BDBM50091347, a CB₁ agent, and 5-CT. In embodiments, the combination comprises BDBM50091347, a CB₁ agent, and 5-MeO-DALT. In embodiments, the combination comprises BDBM50091347, a CB₁ agent, and 5-MeO-DMT. In embodiments, the combination comprises BDBM50091347, a CB₁ agent, and 5-MeO-MiPT. In embodiments, the combination comprises BDBM50091347, a CB₁ agent, and AGH-192. In embodiments, the combination comprises BDBM50091347, a CB₁ agent, and AGH-107. In embodiments, the combination comprises BDBM50091347, a CB₁ agent, and AMT. In embodiments, the combination comprises BDBM50091347, a CB₁ agent, and aripiprazole. In embodiments, the combination comprises BDBM50091347, a CB₁ agent, and AS-19. In embodiments, the combination comprises BDBM50091347, a CB₁ agent, and E-55888. In embodiments, the combination comprises BDBM50091347, a CB₁ agent, and LSD. In embodiments, the combination comprises BDBM50091347, a CB₁ agent, and LP-211. In embodiments, the combination comprises BDBM50091347, a CB₁ agent, and LP-44.

In embodiments, the combination comprises clonidine, a CB₁ agent, and 1-methylpsilocin. In embodiments, the combination comprises clonidine, a CB₁ agent, and 2C-B-FLY. In embodiments, the combination comprises clonidine, a CB₁ agent, and 5-MeO-AMT. In embodiments, the combination comprises clonidine, a CB₁ agent, and AL-37350A. In embodiments, the combination comprises clonidine, a CB₁ agent, and CP 809101. In embodiments, the combination comprises clonidine, a CB₁ agent, and DALT. In embodiments, the combination comprises clonidine, a CB₁ agent, and DOB. In embodiments, the combination comprises clonidine, a CB₁ agent, and DOET In embodiments, the combination comprises clonidine, a CB₁ agent, and DOHx. In embodiments, the combination comprises clonidine, a CB₁ agent, and MEM. In embodiments, the combination comprises clonidine, a CB₁ agent, and CPP. In embodiments, the combination comprises clonidine, a CB₁ agent, and NBOH-2C-CN. In embodiments, the combination comprises clonidine, a CB₁ agent, and TCB-2. In embodiments, the combination comprises clonidine, a CB₁ agent, and WAY 161503. In embodiments, the combination comprises clonidine, a CB₁ agent, and 5-CT. In embodiments, the combination comprises clonidine, a CB₁ agent, and 5-MeO-DALT. In embodiments, the combination comprises clonidine, a CB₁ agent, and 5-MeO-DMT. In embodiments, the combination comprises clonidine, a CB₁ agent, and 5-MeO-MiPT. In embodiments, the combination comprises clonidine, a CB₁ agent, and AGH-192. In embodiments, the combination comprises clonidine, a CB₁ agent, and AGH-107. In embodiments, the combination comprises clonidine, a CB₁ agent, and AMT. In embodiments, the combination comprises clonidine, a CB₁ agent, and aripiprazole. In embodiments, the combination comprises clonidine, a CB₁ agent, and AS-19. In embodiments, the combination comprises clonidine, a CB₁ agent, and E-55888. In embodiments, the combination comprises clonidine, a CB₁ agent, and LSD. In embodiments, the combination comprises clonidine, a CB₁ agent, and LP-211. In embodiments, the combination comprises clonidine, a CB₁ agent, and LP-44.

In embodiments, the combination comprises guanfacine, a CB₁ agent, and 1-methylpsilocin. In embodiments, the combination comprises guanfacine, a CB₁ agent, and 2C-B-FLY. In embodiments, the combination comprises guanfacine, a CB₁ agent, and 5-MeO-AMT. In embodiments, the combination comprises guanfacine, a CB₁ agent, and AL-37350A. In embodiments, the combination comprises guanfacine, a CB₁ agent, and CP 809101. In embodiments, the combination comprises guanfacine, a CB₁ agent, and DALT. In embodiments, the combination comprises guanfacine, a CB₁ agent, and DOB. In embodiments, the combination comprises guanfacine, a CB₁ agent, and DOET In embodiments, the combination comprises guanfacine, a CB₁ agent, and DOHx. In embodiments, the combination comprises guanfacine, a CB₁ agent, and MEM. In embodiments, the combination comprises guanfacine, a CB₁ agent, and CPP. In embodiments, the combination comprises guanfacine, a CB₁ agent, and NBOH-2C-CN. In embodiments, the combination comprises guanfacine, a CB₁ agent, and TCB-2. In embodiments, the combination comprises guanfacine, a CB₁ agent, and WAY 161503. In embodiments, the combination comprises guanfacine, a CB₁ agent, and 5-CT. In embodiments, the combination comprises guanfacine, a CB₁ agent, and 5-MeO-DALT. In embodiments, the combination comprises guanfacine, a CB₁ agent, and 5-MeO-DMT. In embodiments, the combination comprises guanfacine, a CB₁ agent, and 5-MeO-MiPT. In embodiments, the combination comprises guanfacine, a CB₁ agent, and AGH-192. In embodiments, the combination comprises guanfacine, a CB₁ agent, and AGH-107. In embodiments, the combination comprises guanfacine, a CB₁ agent, and AMT. In embodiments, the combination comprises guanfacine, a CB₁ agent, and aripiprazole. In embodiments, the combination comprises guanfacine, a CB₁ agent, and AS-19. In embodiments, the combination comprises guanfacine, a CB₁ agent, and E-55888. In embodiments, the combination comprises guanfacine, a CB₁ agent, and LSD. In embodiments, the combination comprises guanfacine, a CB₁ agent, and LP-211. In embodiments, the combination comprises guanfacine, a CB₁ agent, and LP-44.

In embodiments, the combination comprises mCPP, a CB₁ agent, and 1-methylpsilocin. In embodiments, the combination comprises mCPP, a CB₁ agent, and 2C-B-FLY. In embodiments, the combination comprises mCPP, a CB₁ agent, and 5-MeO-AMT. In embodiments, the combination comprises mCPP, a CB₁ agent, and AL-37350A. In embodiments, the combination comprises mCPP, a CB₁ agent, and CP 809101. In embodiments, the combination comprises mCPP, a CB₁ agent, and DALT. In embodiments, the combination comprises mCPP, a CB₁ agent, and DOB. In embodiments, the combination comprises mCPP, a CB₁ agent, and DOET In embodiments, the combination comprises mCPP, a CB₁ agent, and DOHx. In embodiments, the combination comprises mCPP, a CB₁ agent, and MEM. In embodiments, the combination comprises mCPP, a CB₁ agent, and CPP. In embodiments, the combination comprises mCPP, a CB₁ agent, and NBOH-2C-CN. In embodiments, the combination comprises mCPP, a CB₁ agent, and TCB-2. In embodiments, the combination comprises mCPP, a CB₁ agent, and WAY 161503. In embodiments, the combination comprises mCPP, a CB₁ agent, and 5-CT. In embodiments, the combination comprises mCPP, a CB₁ agent, and 5-MeO-DALT. In embodiments, the combination comprises mCPP, a CB₁ agent, and 5-MeO-DMT. In embodiments, the combination comprises mCPP, a CB₁ agent, and 5-MeO-MiPT. In embodiments, the combination comprises mCPP, a CB₁ agent, and AGH-192. In embodiments, the combination comprises mCPP, a CB₁ agent, and AGH-107. In embodiments, the combination comprises mCPP, a CB₁ agent, and AMT. In embodiments, the combination comprises mCPP, a CB₁ agent, and aripiprazole. In embodiments, the combination comprises mCPP, a CB₁ agent, and AS-19. In embodiments, the combination comprises mCPP, a CB₁ agent, and E-55888. In embodiments, the combination comprises mCPP, a CB₁ agent, and LSD. In embodiments, the combination comprises mCPP, a CB₁ agent, and LP-211. In embodiments, the combination comprises mCPP, a CB₁ agent, and LP-44.

In embodiments, the combination comprises MDMA, a CB₁ agent, and 1-methylpsilocin. In embodiments, the combination comprises MDMA, a CB₁ agent, and 2C-B-FLY. In embodiments, the combination comprises MDMA, a CB₁ agent, and 5-MeO-AMT. In embodiments, the combination comprises MDMA, a CB₁ agent, and AL-37350A. In embodiments, the combination comprises MDMA, a CB₁ agent, and CP 809101. In embodiments, the combination comprises MDMA, a CB₁ agent, and DALT. In embodiments, the combination comprises MDMA, a CB₁ agent, and DOB. In embodiments, the combination comprises MDMA, a CB₁ agent, and DOET. In embodiments, the combination comprises MDMA, a CB₁ agent, and DOHx. In embodiments, the combination comprises MDMA, a CB₁ agent, and MEM. In embodiments, the combination comprises MDMA, a CB₁ agent, and CPP. In embodiments, the combination comprises MDMA, a CB₁ agent, and NBOH-2C-CN. In embodiments, the combination comprises MDMA, a CB₁ agent, and TCB-2. In embodiments, the combination comprises MDMA, a CB₁ agent, and WAY 161503. In embodiments, the combination comprises MDMA, a CB₁ agent, and 5-CT. In embodiments, the combination comprises MDMA, a CB₁ agent, and 5-MeO-DALT. In embodiments, the combination comprises MDMA, a CB₁ agent, and 5-MeO-DMT In embodiments, the combination comprises MDMA, a CB₁ agent, and 5-MeO-MiPT. In embodiments, the combination comprises MDMA, a CB₁ agent, and AGH-192. In embodiments, the combination comprises MDMA, a CB₁ agent, and AGH-107. In embodiments, the combination comprises MDMA, a CB₁ agent, and AMT. In embodiments, the combination comprises MDMA, a CB₁ agent, and aripiprazole. In embodiments, the combination comprises MDMA, a CB₁ agent, and AS-19. In embodiments, the combination comprises MDMA, a CB₁ agent, and E-55888. In embodiments, the combination comprises MDMA, a CB₁ agent, and LSD. In embodiments, the combination comprises MDMA, a CB₁ agent, and LP-211. In embodiments, the combination comprises MDMA, a CB₁ agent, and LP-44.

In embodiments, the combination comprises memantine, a CB₁ agent, and 1-methylpsilocin. In embodiments, the combination comprises memantine, a CB₁ agent, and 2C-B-FLY. In embodiments, the combination comprises memantine, a CB₁ agent, and 5-MeO-AMT. In embodiments, the combination comprises memantine, a CB₁ agent, and AL-37350A. In embodiments, the combination comprises memantine, a CB₁ agent, and CP 809101. In embodiments, the combination comprises memantine, a CB₁ agent, and DALT. In embodiments, the combination comprises memantine, a CB₁ agent, and DOB. In embodiments, the combination comprises memantine, a CB₁ agent, and DOET In embodiments, the combination comprises memantine, a CB₁ agent, and DOHx. In embodiments, the combination comprises memantine, a CB₁ agent, and MEM. In embodiments, the combination comprises memantine, a CB₁ agent, and CPP. In embodiments, the combination comprises memantine, a CB₁ agent, and NBOH-2C-CN. In embodiments, the combination comprises memantine, a CB₁ agent, and TCB-2. In embodiments, the combination comprises memantine, a CB₁ agent, and WAY 161503. In embodiments, the combination comprises memantine, a CB₁ agent, and 5-CT. In embodiments, the combination comprises memantine, a CB₁ agent, and 5-MeO-DALT. In embodiments, the combination comprises memantine, a CB₁ agent, and 5-MeO-DMT. In embodiments, the combination comprises memantine, a CB₁ agent, and 5-MeO-MiPT In embodiments, the combination comprises memantine, a CB₁ agent, and AGH-192. In embodiments, the combination comprises memantine, a CB₁ agent, and AGH-107. In embodiments, the combination comprises memantine, a CB₁ agent, and AMT. In embodiments, the combination comprises memantine, a CB₁ agent, and aripiprazole. In embodiments, the combination comprises memantine, a CB₁ agent, and AS-19. In embodiments, the combination comprises memantine, a CB₁ agent, and E-55888. In embodiments, the combination comprises memantine, a CB₁ agent, and LSD. In embodiments, the combination comprises memantine, a CB₁ agent, and LP-211. In embodiments, the combination comprises memantine, a CB₁ agent, and LP-44.

In embodiments, the combination comprises moxonidine, a CB₁ agent, and 1-methylpsilocin. In embodiments, the combination comprises moxonidine, a CB₁ agent, and 2C-B-FLY. In embodiments, the combination comprises moxonidine, a CB₁ agent, and 5-MeO-AMT. In embodiments, the combination comprises moxonidine, a CB₁ agent, and AL-37350A. In embodiments, the combination comprises moxonidine, a CB₁ agent, and CP 809101. In embodiments, the combination comprises moxonidine, a CB₁ agent, and DALT. In embodiments, the combination comprises moxonidine, a CB₁ agent, and DOB. In embodiments, the combination comprises moxonidine, a CB₁ agent, and DOET In embodiments, the combination comprises moxonidine, a CB₁ agent, and DOHx. In embodiments, the combination comprises moxonidine, a CB₁ agent, and MEM. In embodiments, the combination comprises moxonidine, a CB₁ agent, and CPP. In embodiments, the combination comprises moxonidine, a CB₁ agent, and NBOH-2C-CN. In embodiments, the combination comprises moxonidine, a CB₁ agent, and TCB-2. In embodiments, the combination comprises moxonidine, a CB₁ agent, and WAY 161503. In embodiments, the combination comprises moxonidine, a CB₁ agent, and 5-CT. In embodiments, the combination comprises moxonidine, a CB₁ agent, and 5-MeO-DALT. In embodiments, the combination comprises moxonidine, a CB₁ agent, and 5-MeO-DMT. In embodiments, the combination comprises moxonidine, a CB₁ agent, and 5-MeO-MiPT. In embodiments, the combination comprises moxonidine, a CB₁ agent, and AGH-192. In embodiments, the combination comprises moxonidine, a CB₁ agent, and AGH-107. In embodiments, the combination comprises moxonidine, a CB₁ agent, and AMT. In embodiments, the combination comprises moxonidine, a CB₁ agent, and aripiprazole. In embodiments, the combination comprises moxonidine, a CB₁ agent, and AS-19. In embodiments, the combination comprises moxonidine, a CB₁ agent, and E-55888. In embodiments, the combination comprises moxonidine, a CB₁ agent, and LSD. In embodiments, the combination comprises moxonidine, a CB₁ agent, and LP-211. In embodiments, the combination comprises moxonidine, a CB₁ agent, and LP-44.

In embodiments, the combination comprises naphazoline, a CB₁ agent, and 1-methylpsilocin. In embodiments, the combination comprises naphazoline, a CB₁ agent, and 2C-B-FLY. In embodiments, the combination comprises naphazoline, a CB₁ agent, and 5-MeO-AMT. In embodiments, the combination comprises naphazoline, a CB₁ agent, and AL-37350A. In embodiments, the combination comprises naphazoline, a CB₁ agent, and CP 809101. In embodiments, the combination comprises naphazoline, a CB₁ agent, and DALT. In embodiments, the combination comprises naphazoline, a CB₁ agent, and DOB. In embodiments, the combination comprises naphazoline, a CB₁ agent, and DOET. In embodiments, the combination comprises naphazoline, a CB₁ agent, and DOHx. In embodiments, the combination comprises naphazoline, a CB₁ agent, and MEM. In embodiments, the combination comprises naphazoline, a CB₁ agent, and CPP. In embodiments, the combination comprises naphazoline, a CB₁ agent, and NBOH-2C-CN. In embodiments, the combination comprises naphazoline, a CB₁ agent, and TCB-2. In embodiments, the combination comprises naphazoline, a CB₁ agent, and WAY 161503. In embodiments, the combination comprises naphazoline, a CB₁ agent, and 5-CT. In embodiments, the combination comprises naphazoline, a CB₁ agent, and 5-MeO-DALT. In embodiments, the combination comprises naphazoline, a CB₁ agent, and 5-MeO-DMT. In embodiments, the combination comprises naphazoline, a CB₁ agent, and 5-MeO-MiPT. In embodiments, the combination comprises naphazoline, a CB₁ agent, and AGH-192. In embodiments, the combination comprises naphazoline, a CB₁ agent, and AGH-107. In embodiments, the combination comprises naphazoline, a CB₁ agent, and AMT. In embodiments, the combination comprises naphazoline, a CB₁ agent, and aripiprazole. In embodiments, the combination comprises naphazoline, a CB₁ agent, and AS-19. In embodiments, the combination comprises naphazoline, a CB₁ agent, and E-55888. In embodiments, the combination comprises naphazoline, a CB₁ agent, and LSD. In embodiments, the combination comprises naphazoline, a CB₁ agent, and LP-211. In embodiments, the combination comprises naphazoline, a CB₁ agent, and LP-44.

In embodiments, the combination comprises oxymetazoline, a CB₁ agent, and 1-methylpsilocin. In embodiments, the combination comprises oxymetazoline, a CB₁ agent, and 2C-B-FLY. In embodiments, the combination comprises oxymetazoline, a CB₁ agent, and 5-MeO-AMT. In embodiments, the combination comprises oxymetazoline, a CB₁ agent, and AL-37350A. In embodiments, the combination comprises oxymetazoline, a CB₁ agent, and CP 809101. In embodiments, the combination comprises oxymetazoline, a CB₁ agent, and DALT. In embodiments, the combination comprises oxymetazoline, a CB₁ agent, and DOB. In embodiments, the combination comprises oxymetazoline, a CB₁ agent, and DOET In embodiments, the combination comprises oxymetazoline, a CB₁ agent, and DOHx. In embodiments, the combination comprises oxymetazoline, a CB₁ agent, and MEM. In embodiments, the combination comprises oxymetazoline, a CB₁ agent, and CPP. In embodiments, the combination comprises oxymetazoline, a CB₁ agent, and NBOH-2C-CN. In embodiments, the combination comprises oxymetazoline, a CB₁ agent, and TCB-2. In embodiments, the combination comprises oxymetazoline, a CB₁ agent, and WAY 161503. In embodiments, the combination comprises oxymetazoline, a CB₁ agent, and 5-CT. In embodiments, the combination comprises oxymetazoline, a CB₁ agent, and 5-MeO-DALT. In embodiments, the combination comprises oxymetazoline, a CB₁ agent, and 5-MeO-DMT In embodiments, the combination comprises oxymetazoline, a CB₁ agent, and 5-MeO-MiPT. In embodiments, the combination comprises oxymetazoline, a CB₁ agent, and AGH-192. In embodiments, the combination comprises oxymetazoline, a CB₁ agent, and AGH-107. In embodiments, the combination comprises oxymetazoline, a CB₁ agent, and AMT. In embodiments, the combination comprises oxymetazoline, a CB₁ agent, and aripiprazole. In embodiments, the combination comprises oxymetazoline, a CB₁ agent, and AS-19. In embodiments, the combination comprises oxymetazoline, a CB₁ agent, and E-55888. In embodiments, the combination comprises oxymetazoline, a CB₁ agent, and LSD. In embodiments, the combination comprises oxymetazoline, a CB₁ agent, and LP-211. In embodiments, the combination comprises oxymetazoline, a CB₁ agent, and LP-44.

In embodiments, the combination comprises rilmenidine, a CB₁ agent, and 1-methylpsilocin. In embodiments, the combination comprises rilmenidine, a CB₁ agent, and 2C-B-FLY. In embodiments, the combination comprises rilmenidine, a CB₁ agent, and 5-MeO-AMT. In embodiments, the combination comprises rilmenidine, a CB₁ agent, and AL-37350A. In embodiments, the combination comprises rilmenidine, a CB₁ agent, and CP 809101. In embodiments, the combination comprises rilmenidine, a CB₁ agent, and DALT. In embodiments, the combination comprises rilmenidine, a CB₁ agent, and DOB. In embodiments, the combination comprises rilmenidine, a CB₁ agent, and DOET. In embodiments, the combination comprises rilmenidine, a CB₁ agent, and DOHx. In embodiments, the combination comprises rilmenidine, a CB₁ agent, and MEM. In embodiments, the combination comprises rilmenidine, a CB₁ agent, and CPP. In embodiments, the combination comprises rilmenidine, a CB₁ agent, and NBOH-2C-CN. In embodiments, the combination comprises rilmenidine, a CB₁ agent, and TCB-2. In embodiments, the combination comprises rilmenidine, a CB₁ agent, and WAY 161503. In embodiments, the combination comprises rilmenidine, a CB₁ agent, and 5-CT. In embodiments, the combination comprises rilmenidine, a CB₁ agent, and 5-MeO-DALT. In embodiments, the combination comprises rilmenidine, a CB₁ agent, and 5-MeO-DMT. In embodiments, the combination comprises rilmenidine, a CB₁ agent, and 5-MeO-MiPT. In embodiments, the combination comprises rilmenidine, a CB₁ agent, and AGH-192. In embodiments, the combination comprises rilmenidine, a CB₁ agent, and AGH-107. In embodiments, the combination comprises rilmenidine, a CB₁ agent, and AMT. In embodiments, the combination comprises rilmenidine, a CB₁ agent, and aripiprazole. In embodiments, the combination comprises rilmenidine, a CB₁ agent, and AS-19. In embodiments, the combination comprises rilmenidine, a CB₁ agent, and E-55888. In embodiments, the combination comprises rilmenidine, a CB₁ agent, and LSD. In embodiments, the combination comprises rilmenidine, a CB₁ agent, and LP-211. In embodiments, the combination comprises rilmenidine, a CB₁ agent, and LP-44.

In embodiments, the combination comprises tetryzoline, a CB₁ agent, and 1-methylpsilocin. In embodiments, the combination comprises tetryzoline, a CB₁ agent, and 2C-B-FLY. In embodiments, the combination comprises tetryzoline, a CB₁ agent, and 5-MeO-AMT. In embodiments, the combination comprises tetryzoline, a CB₁ agent, and AL-37350A. In embodiments, the combination comprises tetryzoline, a CB₁ agent, and CP 809101. In embodiments, the combination comprises tetryzoline, a CB₁ agent, and DALT. In embodiments, the combination comprises tetryzoline, a CB₁ agent, and DOB. In embodiments, the combination comprises tetryzoline, a CB₁ agent, and DOET. In embodiments, the combination comprises tetryzoline, a CB₁ agent, and DOHx. In embodiments, the combination comprises tetryzoline, a CB₁ agent, and MEM. In embodiments, the combination comprises tetryzoline, a CB₁ agent, and CPP. In embodiments, the combination comprises tetryzoline, a CB₁ agent, and NBOH-2C-CN. In embodiments, the combination comprises tetryzoline, a CB₁ agent, and TCB-2. In embodiments, the combination comprises tetryzoline, a CB₁ agent, and WAY 161503. In embodiments, the combination comprises tetryzoline, a CB₁ agent, and 5-CT. In embodiments, the combination comprises tetryzoline, a CB₁ agent, and 5-MeO-DALT. In embodiments, the combination comprises tetryzoline, a CB₁ agent, and 5-MeO-DMT. In embodiments, the combination comprises tetryzoline, a CB₁ agent, and 5-MeO-MiPT. In embodiments, the combination comprises tetryzoline, a CB₁ agent, and AGH-192. In embodiments, the combination comprises tetryzoline, a CB₁ agent, and AGH-107. In embodiments, the combination comprises tetryzoline, a CB₁ agent, and AMT. In embodiments, the combination comprises tetryzoline, a CB₁ agent, and aripiprazole. In embodiments, the combination comprises tetryzoline, a CB₁ agent, and AS-19. In embodiments, the combination comprises tetryzoline, a CB₁ agent, and E-55888. In embodiments, the combination comprises tetryzoline, a CB₁ agent, and LSD. In embodiments, the combination comprises tetryzoline, a CB₁ agent, and LP-211. In embodiments, the combination comprises tetryzoline, a CB₁ agent, and LP-44.

In embodiments, the combination comprises tizanidine, a CB₁ agent, and 1-methylpsilocin. In embodiments, the combination comprises tizanidine, a CB₁ agent, and 2C-B-FLY. In embodiments, the combination comprises tizanidine, a CB₁ agent, and 5-MeO-AMT. In embodiments, the combination comprises tizanidine, a CB₁ agent, and AL-37350A. In embodiments, the combination comprises tizanidine, a CB₁ agent, and CP 809101. In embodiments, the combination comprises tizanidine, a CB₁ agent, and DALT. In embodiments, the combination comprises tizanidine, a CB₁ agent, and DOB. In embodiments, the combination comprises tizanidine, a CB₁ agent, and DOET In embodiments, the combination comprises tizanidine, a CB₁ agent, and DOHx. In embodiments, the combination comprises tizanidine, a CB₁ agent, and MEM. In embodiments, the combination comprises tizanidine, a CB₁ agent, and CPP. In embodiments, the combination comprises tizanidine, a CB₁ agent, and NBOH-2C-CN. In embodiments, the combination comprises tizanidine, a CB₁ agent, and TCB-2. In embodiments, the combination comprises tizanidine, a CB₁ agent, and WAY 161503. In embodiments, the combination comprises tizanidine, a CB₁ agent, and 5-CT. In embodiments, the combination comprises tizanidine, a CB₁ agent, and 5-MeO-DALT. In embodiments, the combination comprises tizanidine, a CB₁ agent, and 5-MeO-DMT In embodiments, the combination comprises tizanidine, a CB₁ agent, and 5-MeO-MiPT. In embodiments, the combination comprises tizanidine, a CB₁ agent, and AGH-192. In embodiments, the combination comprises tizanidine, a CB₁ agent, and AGH-107. In embodiments, the combination comprises tizanidine, a CB₁ agent, and AMT. In embodiments, the combination comprises tizanidine, a CB₁ agent, and aripiprazole. In embodiments, the combination comprises tizanidine, a CB₁ agent, and AS-19. In embodiments, the combination comprises tizanidine, a CB₁ agent, and E-55888. In embodiments, the combination comprises tizanidine, a CB₁ agent, and LSD. In embodiments, the combination comprises tizanidine, a CB₁ agent, and LP-211. In embodiments, the combination comprises tizanidine, a CB₁ agent, and LP-44.

In embodiments, the combination comprises tolonidine, a CB₁ agent, and 1-methylpsilocin. In embodiments, the combination comprises tolonidine, a CB₁ agent, and 2C-B-FLY. In embodiments, the combination comprises tolonidine, a CB₁ agent, and 5-MeO-AMT. In embodiments, the combination comprises tolonidine, a CB₁ agent, and AL-37350A. In embodiments, the combination comprises tolonidine, a CB₁ agent, and CP 809101. In embodiments, the combination comprises tolonidine, a CB₁ agent, and DALT. In embodiments, the combination comprises tolonidine, a CB₁ agent, and DOB. In embodiments, the combination comprises tolonidine, a CB₁ agent, and DOET. In embodiments, the combination comprises tolonidine, a CB₁ agent, and DOHx. In embodiments, the combination comprises tolonidine, a CB₁ agent, and MEM. In embodiments, the combination comprises tolonidine, a CB₁ agent, and CPP. In embodiments, the combination comprises tolonidine, a CB₁ agent, and NBOH-2C-CN. In embodiments, the combination comprises tolonidine, a CB₁ agent, and TCB-2. In embodiments, the combination comprises tolonidine, a CB₁ agent, and WAY 161503. In embodiments, the combination comprises tolonidine, a CB₁ agent, and 5-CT. In embodiments, the combination comprises tolonidine, a CB₁ agent, and 5-MeO-DALT. In embodiments, the combination comprises tolonidine, a CB₁ agent, and 5-MeO-DMT In embodiments, the combination comprises tolonidine, a CB₁ agent, and 5-MeO-MiPT. In embodiments, the combination comprises tolonidine, a CB₁ agent, and AGH-192. In embodiments, the combination comprises tolonidine, a CB₁ agent, and AGH-107. In embodiments, the combination comprises tolonidine, a CB₁ agent, and AMT. In embodiments, the combination comprises tolonidine, a CB₁ agent, and aripiprazole. In embodiments, the combination comprises tolonidine, a CB₁ agent, and AS-19. In embodiments, the combination comprises tolonidine, a CB₁ agent, and E-55888. In embodiments, the combination comprises tolonidine, a CB₁ agent, and LSD. In embodiments, the combination comprises tolonidine, a CB₁ agent, and LP-211. In embodiments, the combination comprises tolonidine, a CB₁ agent, and LP-44.

In embodiments, the combination comprises agmatine, 1-methylpsilocin, and 5-CT. In embodiments, the combination comprises agmatine, 1-methylpsilocin, and 5-MeO-DALT. In embodiments, the combination comprises agmatine, 1-methylpsilocin, and 5-MeO-DMT. In embodiments, the combination comprises agmatine, 1-methylpsilocin, and 5-MeO-MiPT. In embodiments, the combination comprises agmatine, 1-methylpsilocin, and AGH-192. In embodiments, the combination comprises agmatine, 1-methylpsilocin, and AGH-107. In embodiments, the combination comprises agmatine, 1-methylpsilocin, and AMT. In embodiments, the combination comprises agmatine, 1-methylpsilocin, and aripiprazole. In embodiments, the combination comprises agmatine, 1-methylpsilocin, and AS-19. In embodiments, the combination comprises agmatine, 1-methylpsilocin, and E-55888. In embodiments, the combination comprises agmatine, 1-methylpsilocin, and LSD. In embodiments, the combination comprises agmatine, 1-methylpsilocin, and LP-211. In embodiments, the combination comprises agmatine, 1-methylpsilocin, and LP-44.

In embodiments, the combination comprises agmatine, 2C-B-FLY, and 5-CT. In embodiments, the combination comprises agmatine, 2C-B-FLY, and 5-MeO-DALT. In embodiments, the combination comprises agmatine, 2C-B-FLY, and 5-MeO-DMT. In embodiments, the combination comprises agmatine, 2C-B-FLY, and 5-MeO-MiPT. In embodiments, the combination comprises agmatine, 2C-B-FLY, and AGH-192. In embodiments, the combination comprises agmatine, 2C-B-FLY, and AGH-107. In embodiments, the combination comprises agmatine, 2C-B-FLY, and AMT. In embodiments, the combination comprises agmatine, 2C-B-FLY, and aripiprazole. In embodiments, the combination comprises agmatine, 2C-B-FLY, and AS-19. In embodiments, the combination comprises agmatine, 2C-B-FLY, and E-55888. In embodiments, the combination comprises agmatine, 2C-B-FLY, and LSD. In embodiments, the combination comprises agmatine, 2C-B-FLY, and LP-211. In embodiments, the combination comprises agmatine, 2C-B-FLY, and LP-44.

In embodiments, the combination comprises agmatine, 5-MeO-AMT, and 5-CT. In embodiments, the combination comprises agmatine, 5-MeO-AMT, and 5-MeO-DALT. In embodiments, the combination comprises agmatine, 5-MeO-AMT, and 5-MeO-DMT. In embodiments, the combination comprises agmatine, 5-MeO-AMT, and 5-MeO-MiPT. In embodiments, the combination comprises agmatine, 5-MeO-AMT, and AGH-192. In embodiments, the combination comprises agmatine, 5-MeO-AMT, and AGH-107. In embodiments, the combination comprises agmatine, 5-MeO-AMT, and AMT. In embodiments, the combination comprises agmatine, 5-MeO-AMT, and aripiprazole. In embodiments, the combination comprises agmatine, 5-MeO-AMT, and AS-19. In embodiments, the combination comprises agmatine, 5-MeO-AMT, and E-55888. In embodiments, the combination comprises agmatine, 5-MeO-AMT, and LSD. In embodiments, the combination comprises agmatine, 5-MeO-AMT, and LP-211. In embodiments, the combination comprises agmatine, 5-MeO-AMT, and LP-44.

In embodiments, the combination comprises agmatine, AL-37350A, and 5-CT. In embodiments, the combination comprises agmatine, AL-37350A, and 5-MeO-DALT. In embodiments, the combination comprises agmatine, AL-37350A, and 5-MeO-DMT In embodiments, the combination comprises agmatine, AL-37350A, and 5-MeO-MiPT In embodiments, the combination comprises agmatine, AL-37350A, and AGH-192. In embodiments, the combination comprises agmatine, AL-37350A, and AGH-107. In embodiments, the combination comprises agmatine, AL-37350A, and AMT. In embodiments, the combination comprises agmatine, AL-37350A, and aripiprazole. In embodiments, the combination comprises agmatine, AL-37350A, and AS-19. In embodiments, the combination comprises agmatine, AL-37350A, and E-55888. In embodiments, the combination comprises agmatine, AL-37350A, and LSD. In embodiments, the combination comprises agmatine, AL-37350A, and LP-211. In embodiments, the combination comprises agmatine, AL-37350A, and LP-44.

In embodiments, the combination comprises agmatine, CP 809101, and 5-CT. In embodiments, the combination comprises agmatine, CP 809101, and 5-MeO-DALT. In embodiments, the combination comprises agmatine, CP 809101, and 5-MeO-DMT In embodiments, the combination comprises agmatine, CP 809101, and 5-MeO-MiPT. In embodiments, the combination comprises agmatine, CP 809101, and AGH-192. In embodiments, the combination comprises agmatine, CP 809101, and AGH-107. In embodiments, the combination comprises agmatine, CP 809101, and AMT. In embodiments, the combination comprises agmatine, CP 809101, and aripiprazole. In embodiments, the combination comprises agmatine, CP 809101, and AS-19. In embodiments, the combination comprises agmatine, CP 809101, and E-55888. In embodiments, the combination comprises agmatine, CP 809101, and LSD. In embodiments, the combination comprises agmatine, CP 809101, and LP-211. In embodiments, the combination comprises agmatine, CP 809101, and LP-44.

In embodiments, the combination comprises agmatine, DALT, and 5-CT. In embodiments, the combination comprises agmatine, DALT, and 5-MeO-DALT. In embodiments, the combination comprises agmatine, DALT, and 5-MeO-DMT. In embodiments, the combination comprises agmatine, DALT, and 5-MeO-MiPT. In embodiments, the combination comprises agmatine, DALT, and AGH-192. In embodiments, the combination comprises agmatine, DALT, and AGH-107. In embodiments, the combination comprises agmatine, DALT, and AMT. In embodiments, the combination comprises agmatine, DALT, and aripiprazole. In embodiments, the combination comprises agmatine, DALT, and AS-19. In embodiments, the combination comprises agmatine, DALT, and E-55888. In embodiments, the combination comprises agmatine, DALT, and LSD. In embodiments, the combination comprises agmatine, DALT, and LP-211. In embodiments, the combination comprises agmatine, DALT, and LP-44.

In embodiments, the combination comprises agmatine, DOB, and 5-CT. In embodiments, the combination comprises agmatine, DOB, and 5-MeO-DALT. In embodiments, the combination comprises agmatine, DOB, and 5-MeO-DMT. In embodiments, the combination comprises agmatine, DOB, and 5-MeO-MiPT. In embodiments, the combination comprises agmatine, DOB, and AGH-192. In embodiments, the combination comprises agmatine, DOB, and AGH-107. In embodiments, the combination comprises agmatine, DOB, and AMT. In embodiments, the combination comprises agmatine, DOB, and aripiprazole. In embodiments, the combination comprises agmatine, DOB, and AS-19. In embodiments, the combination comprises agmatine, DOB, and E-55888. In embodiments, the combination comprises agmatine, DOB, and LSD. In embodiments, the combination comprises agmatine, DOB, and LP-211. In embodiments, the combination comprises agmatine, DOB, and LP-44.

In embodiments, the combination comprises agmatine, DOET, and 5-CT. In embodiments, the combination comprises agmatine, DOET, and 5-MeO-DALT. In embodiments, the combination comprises agmatine, DOET, and 5-MeO-DMT. In embodiments, the combination comprises agmatine, DOET, and 5-MeO-MiPT. In embodiments, the combination comprises agmatine, DOET, and AGH-192. In embodiments, the combination comprises agmatine, DOET, and AGH-107. In embodiments, the combination comprises agmatine, DOET, and AMT. In embodiments, the combination comprises agmatine, DOET, and aripiprazole. In embodiments, the combination comprises agmatine, DOET, and AS-19. In embodiments, the combination comprises agmatine, DOET, and E-55888. In embodiments, the combination comprises agmatine, DOET, and LSD. In embodiments, the combination comprises agmatine, DOET, and LP-211. In embodiments, the combination comprises agmatine, DOET, and LP-44.

In embodiments, the combination comprises agmatine, DOHx, and 5-CT. In embodiments, the combination comprises agmatine, DOHx, and 5-MeO-DALT. In embodiments, the combination comprises agmatine, DOHx, and 5-MeO-DMT. In embodiments, the combination comprises agmatine, DOHx, and 5-MeO-MiPT. In embodiments, the combination comprises agmatine, DOHx, and AGH-192. In embodiments, the combination comprises agmatine, DOHx, and AGH-107. In embodiments, the combination comprises agmatine, DOHx, and AMT. In embodiments, the combination comprises agmatine, DOHx, and aripiprazole. In embodiments, the combination comprises agmatine, DOHx, and AS-19. In embodiments, the combination comprises agmatine, DOHx, and E-55888. In embodiments, the combination comprises agmatine, DOHx, and LSD. In embodiments, the combination comprises agmatine, DOHx, and LP-211. In embodiments, the combination comprises agmatine, DOHx, and LP-44.

In embodiments, the combination comprises agmatine, MEM, and 5-CT. In embodiments, the combination comprises agmatine, MEM, and 5-MeO-DALT. In embodiments, the combination comprises agmatine, MEM, and 5-MeO-DMT In embodiments, the combination comprises agmatine, MEM, and 5-MeO-MiPT. In embodiments, the combination comprises agmatine, MEM, and AGH-192. In embodiments, the combination comprises agmatine, MEM, and AGH-107. In embodiments, the combination comprises agmatine, MEM, and AMT. In embodiments, the combination comprises agmatine, MEM, and aripiprazole. In embodiments, the combination comprises agmatine, MEM, and AS-19. In embodiments, the combination comprises agmatine, MEM, and E-55888. In embodiments, the combination comprises agmatine, MEM, and LSD. In embodiments, the combination comprises agmatine, MEM, and LP-211. In embodiments, the combination comprises agmatine, MEM, and LP-44.

In embodiments, the combination comprises agmatine, CPP, and 5-CT. In embodiments, the combination comprises agmatine, CPP, and 5-MeO-DALT. In embodiments, the combination comprises agmatine, CPP, and 5-MeO-DMT. In embodiments, the combination comprises agmatine, CPP, and 5-MeO-MiPT. In embodiments, the combination comprises agmatine, CPP, and AGH-192. In embodiments, the combination comprises agmatine, CPP, and AGH-107. In embodiments, the combination comprises agmatine, CPP, and AMT. In embodiments, the combination comprises agmatine, CPP, and aripiprazole. In embodiments, the combination comprises agmatine, CPP, and AS-19. In embodiments, the combination comprises agmatine, CPP, and E-55888. In embodiments, the combination comprises agmatine, CPP, and LSD. In embodiments, the combination comprises agmatine, CPP, and LP-211. In embodiments, the combination comprises agmatine, CPP, and LP-44.

In embodiments, the combination comprises agmatine, NBOH-2C-CN, and 5-CT. In embodiments, the combination comprises agmatine, NBOH-2C-CN, and 5-MeO-DALT. In embodiments, the combination comprises agmatine, NBOH-2C-CN, and 5-MeO-DMT. In embodiments, the combination comprises agmatine, NBOH-2C-CN, and 5-MeO-MiPT. In embodiments, the combination comprises agmatine, NBOH-2C-CN, and AGH-192. In embodiments, the combination comprises agmatine, NBOH-2C-CN, and AGH-107. In embodiments, the combination comprises agmatine, NBOH-2C-CN, and AMT. In embodiments, the combination comprises agmatine, NBOH-2C-CN, and aripiprazole. In embodiments, the combination comprises agmatine, NBOH-2C-CN, and AS-19. In embodiments, the combination comprises agmatine, NBOH-2C-CN, and E-55888. In embodiments, the combination comprises agmatine, NBOH-2C-CN, and LSD. In embodiments, the combination comprises agmatine, NBOH-2C-CN, and LP-211. In embodiments, the combination comprises agmatine, NBOH-2C-CN, and LP-44.

In embodiments, the combination comprises agmatine, TCB-2, and 5-CT. In embodiments, the combination comprises agmatine, TCB-2, and 5-MeO-DALT. In embodiments, the combination comprises agmatine, TCB-2, and 5-MeO-DMT. In embodiments, the combination comprises agmatine, TCB-2, and 5-MeO-MiPT. In embodiments, the combination comprises agmatine, TCB-2, and AGH-192. In embodiments, the combination comprises agmatine, TCB-2, and AGH-107. In embodiments, the combination comprises agmatine, TCB-2, and AMT. In embodiments, the combination comprises agmatine, TCB-2, and aripiprazole. In embodiments, the combination comprises agmatine, TCB-2, and AS-19. In embodiments, the combination comprises agmatine, TCB-2, and E-55888. In embodiments, the combination comprises agmatine, TCB-2, and LSD. In embodiments, the combination comprises agmatine, TCB-2, and LP-211. In embodiments, the combination comprises agmatine, TCB-2, and LP-44.

In embodiments, the combination comprises agmatine, WAY 161503, and 5-CT. In embodiments, the combination comprises agmatine, WAY 161503, and 5-MeO-DALT. In embodiments, the combination comprises agmatine, WAY 161503, and 5-MeO-DMT. In embodiments, the combination comprises agmatine, WAY 161503, and 5-MeO-MiPT. In embodiments, the combination comprises agmatine, WAY 161503, and AGH-192. In embodiments, the combination comprises agmatine, WAY 161503, and AGH-107. In embodiments, the combination comprises agmatine, WAY 161503, and AMT. In embodiments, the combination comprises agmatine, WAY 161503, and aripiprazole. In embodiments, the combination comprises agmatine, WAY 161503, and AS-19. In embodiments, the combination comprises agmatine, WAY 161503, and E-55888. In embodiments, the combination comprises agmatine, WAY 161503, and LSD. In embodiments, the combination comprises agmatine, WAY 161503, and LP-211. In embodiments, the combination comprises agmatine, WAY 161503, and LP-44.

In embodiments, the combination comprises BDBM50091347, 1-methylpsilocin, and 5-CT. In embodiments, the combination comprises BDBM50091347, 1-methylpsilocin, and 5-MeO-DALT In embodiments, the combination comprises BDBM50091347, 1-methylpsilocin, and 5-MeO-DMT. In embodiments, the combination comprises BDBM50091347, 1-methylpsilocin, and 5-MeO-MiPT. In embodiments, the combination comprises BDBM50091347, 1-methylpsilocin, and AGH-192. In embodiments, the combination comprises BDBM50091347, 1-methylpsilocin, and AGH-107. In embodiments, the combination comprises BDBM50091347, 1-methylpsilocin, and AMT. In embodiments, the combination comprises BDBM50091347, 1-methylpsilocin, and aripiprazole. In embodiments, the combination comprises BDBM50091347, 1-methylpsilocin, and AS-19. In embodiments, the combination comprises BDBM50091347, 1-methylpsilocin, and E-55888. In embodiments, the combination comprises BDBM50091347, 1-methylpsilocin, and LSD. In embodiments, the combination comprises BDBM50091347, 1-methylpsilocin, and LP-211. In embodiments, the combination comprises BDBM50091347, 1-methylpsilocin, and LP-44.

In embodiments, the combination comprises BDBM50091347, 2C-B-FLY, and 5-CT. In embodiments, the combination comprises BDBM50091347, 2C-B-FLY, and 5-MeO-DALT In embodiments, the combination comprises BDBM50091347, 2C-B-FLY, and 5-MeO-DMT In embodiments, the combination comprises BDBM50091347, 2C-B-FLY, and 5-MeO-MiPT. In embodiments, the combination comprises BDBM50091347, 2C-B-FLY, and AGH-192. In embodiments, the combination comprises BDBM50091347, 2C-B-FLY, and AGH-107. In embodiments, the combination comprises BDBM50091347, 2C-B-FLY, and AMT. In embodiments, the combination comprises BDBM50091347, 2C-B-FLY, and aripiprazole. In embodiments, the combination comprises BDBM50091347, 2C-B-FLY, and AS-19. In embodiments, the combination comprises BDBM50091347, 2C-B-FLY, and E-55888. In embodiments, the combination comprises BDBM50091347, 2C-B-FLY, and LSD. In embodiments, the combination comprises BDBM50091347, 2C-B-FLY, and LP-211. In embodiments, the combination comprises BDBM50091347, 2C-B-FLY, and LP-44.

In embodiments, the combination comprises BDBM50091347, 5-MeO-AMT, and 5-CT. In embodiments, the combination comprises BDBM50091347, 5-MeO-AMT, and 5-MeO-DALT. In embodiments, the combination comprises BDBM50091347, 5-MeO-AMT, and 5-MeO-DMT. In embodiments, the combination comprises BDBM50091347, 5-MeO-AMT, and 5-MeO-MiPT In embodiments, the combination comprises BDBM50091347, 5-MeO-AMT, and AGH-192. In embodiments, the combination comprises BDBM50091347, 5-MeO-AMT, and AGH-107. In embodiments, the combination comprises BDBM50091347, 5-MeO-AMT, and AMT. In embodiments, the combination comprises BDBM50091347, 5-MeO-AMT, and aripiprazole. In embodiments, the combination comprises BDBM50091347, 5-MeO-AMT, and AS-19. In embodiments, the combination comprises BDBM50091347, 5-MeO-AMT, and E-55888. In embodiments, the combination comprises BDBM50091347, 5-MeO-AMT, and LSD. In embodiments, the combination comprises BDBM50091347, 5-MeO-AMT, and LP-211. In embodiments, the combination comprises BDBM50091347, 5-MeO-AMT, and LP-44.

In embodiments, the combination comprises BDBM50091347, AL-37350A, and 5-CT. In embodiments, the combination comprises BDBM50091347, AL-37350A, and 5-MeO-DALT. In embodiments, the combination comprises BDBM50091347, AL-37350A, and 5-MeO-DMT. In embodiments, the combination comprises BDBM50091347, AL-37350A, and 5-MeO-MiPT. In embodiments, the combination comprises BDBM50091347, AL-37350A, and AGH-192. In embodiments, the combination comprises BDBM50091347, AL-37350A, and AGH-107. In embodiments, the combination comprises BDBM50091347, AL-37350A, and AMT. In embodiments, the combination comprises BDBM50091347, AL-37350A, and aripiprazole. In embodiments, the combination comprises BDBM50091347, AL-37350A, and AS-19. In embodiments, the combination comprises BDBM50091347, AL-37350A, and E-55888. In embodiments, the combination comprises BDBM50091347, AL-37350A, and LSD. In embodiments, the combination comprises BDBM50091347, AL-37350A, and LP-211. In embodiments, the combination comprises BDBM50091347, AL-37350A, and LP-44.

In embodiments, the combination comprises BDBM50091347, CP 809101, and 5-CT. In embodiments, the combination comprises BDBM50091347, CP 809101, and 5-MeO-DALT. In embodiments, the combination comprises BDBM50091347, CP 809101, and 5-MeO-DMT. In embodiments, the combination comprises BDBM50091347, CP 809101, and 5-MeO-MiPT. In embodiments, the combination comprises BDBM50091347, CP 809101, and AGH-192. In embodiments, the combination comprises BDBM50091347, CP 809101, and AGH-107. In embodiments, the combination comprises BDBM50091347, CP 809101, and AMT. In embodiments, the combination comprises BDBM50091347, CP 809101, and aripiprazole. In embodiments, the combination comprises BDBM50091347, CP 809101, and AS-19. In embodiments, the combination comprises BDBM50091347, CP 809101, and E-55888. In embodiments, the combination comprises BDBM50091347, CP 809101, and LSD. In embodiments, the combination comprises BDBM50091347, CP 809101, and LP-211. In embodiments, the combination comprises BDBM50091347, CP 809101, and LP-44.

In embodiments, the combination comprises BDBM50091347, DALT, and 5-CT. In embodiments, the combination comprises BDBM50091347, DALT, and 5-MeO-DALT. In embodiments, the combination comprises BDBM50091347, DALT, and 5-MeO-DMT. In embodiments, the combination comprises BDBM50091347, DALT, and 5-MeO-MiPT In embodiments, the combination comprises BDBM50091347, DALT, and AGH-192. In embodiments, the combination comprises BDBM50091347, DALT, and AGH-107. In embodiments, the combination comprises BDBM50091347, DALT, and AMT. In embodiments, the combination comprises BDBM50091347, DALT, and aripiprazole. In embodiments, the combination comprises BDBM50091347, DALT, and AS-19. In embodiments, the combination comprises BDBM50091347, DALT, and E-55888. In embodiments, the combination comprises BDBM50091347, DALT, and LSD. In embodiments, the combination comprises BDBM50091347, DALT, and LP-211. In embodiments, the combination comprises BDBM50091347, DALT, and LP-44.

In embodiments, the combination comprises BDBM50091347, DOB, and 5-CT. In embodiments, the combination comprises BDBM50091347, DOB, and 5-MeO-DALT. In embodiments, the combination comprises BDBM50091347, DOB, and 5-MeO-DMT In embodiments, the combination comprises BDBM50091347, DOB, and 5-MeO-MiPT In embodiments, the combination comprises BDBM50091347, DOB, and AGH-192. In embodiments, the combination comprises BDBM50091347, DOB, and AGH-107. In embodiments, the combination comprises BDBM50091347, DOB, and AMT. In embodiments, the combination comprises BDBM50091347, DOB, and aripiprazole. In embodiments, the combination comprises BDBM50091347, DOB, and AS-19. In embodiments, the combination comprises BDBM50091347, DOB, and E-55888. In embodiments, the combination comprises BDBM50091347, DOB, and LSD. In embodiments, the combination comprises BDBM50091347, DOB, and LP-211. In embodiments, the combination comprises BDBM50091347, DOB, and LP-44.

In embodiments, the combination comprises BDBM50091347, DOET, and 5-CT. In embodiments, the combination comprises BDBM50091347, DOET, and 5-MeO-DALT. In embodiments, the combination comprises BDBM50091347, DOET, and 5-MeO-DMT In embodiments, the combination comprises BDBM50091347, DOET, and 5-MeO-MiPT In embodiments, the combination comprises BDBM50091347, DOET, and AGH-192. In embodiments, the combination comprises BDBM50091347, DOET, and AGH-107. In embodiments, the combination comprises BDBM50091347, DOET, and AMT. In embodiments, the combination comprises BDBM50091347, DOET, and aripiprazole. In embodiments, the combination comprises BDBM50091347, DOET, and AS-19. In embodiments, the combination comprises BDBM50091347, DOET, and E-55888. In embodiments, the combination comprises BDBM50091347, DOET, and LSD. In embodiments, the combination comprises BDBM50091347, DOET, and LP-211. In embodiments, the combination comprises BDBM50091347, DOET, and LP-44.

In embodiments, the combination comprises BDBM50091347, DOHx, and 5-CT. In embodiments, the combination comprises BDBM50091347, DOHx, and 5-MeO-DALT. In embodiments, the combination comprises BDBM50091347, DOHx, and 5-MeO-DMT. In embodiments, the combination comprises BDBM50091347, DOHx, and 5-MeO-MiPT. In embodiments, the combination comprises BDBM50091347, DOHx, and AGH-192. In embodiments, the combination comprises BDBM50091347, DOHx, and AGH-107. In embodiments, the combination comprises BDBM50091347, DOHx, and AMT. In embodiments, the combination comprises BDBM50091347, DOHx, and aripiprazole. In embodiments, the combination comprises BDBM50091347, DOHx, and AS-19. In embodiments, the combination comprises BDBM50091347, DOHx, and E-55888. In embodiments, the combination comprises BDBM50091347, DOHx, and LSD. In embodiments, the combination comprises BDBM50091347, DOHx, and LP-211. In embodiments, the combination comprises BDBM50091347, DOHx, and LP-44.

In embodiments, the combination comprises BDBM50091347, MEM, and 5-CT. In embodiments, the combination comprises BDBM50091347, MEM, and 5-MeO-DALT. In embodiments, the combination comprises BDBM50091347, MEM, and 5-MeO-DMT. In embodiments, the combination comprises BDBM50091347, MEM, and 5-MeO-MiPT. In embodiments, the combination comprises BDBM50091347, MEM, and AGH-192. In embodiments, the combination comprises BDBM50091347, MEM, and AGH-107. In embodiments, the combination comprises BDBM50091347, MEM, and AMT. In embodiments, the combination comprises BDBM50091347, MEM, and aripiprazole. In embodiments, the combination comprises BDBM50091347, MEM, and AS-19. In embodiments, the combination comprises BDBM50091347, MEM, and E-55888. In embodiments, the combination comprises BDBM50091347, MEM, and LSD. In embodiments, the combination comprises BDBM50091347, MEM, and LP-211. In embodiments, the combination comprises BDBM50091347, MEM, and LP-44.

In embodiments, the combination comprises BDBM50091347, CPP, and 5-CT. In embodiments, the combination comprises BDBM50091347, CPP, and 5-MeO-DALT. In embodiments, the combination comprises BDBM50091347, CPP, and 5-MeO-DMT. In embodiments, the combination comprises BDBM50091347, CPP, and 5-MeO-MiPT In embodiments, the combination comprises BDBM50091347, CPP, and AGH-192. In embodiments, the combination comprises BDBM50091347, CPP, and AGH-107. In embodiments, the combination comprises BDBM50091347, CPP, and AMT. In embodiments, the combination comprises BDBM50091347, CPP, and aripiprazole. In embodiments, the combination comprises BDBM50091347, CPP, and AS-19. In embodiments, the combination comprises BDBM50091347, CPP, and E-55888. In embodiments, the combination comprises BDBM50091347, CPP, and LSD. In embodiments, the combination comprises BDBM50091347, CPP, and LP-211. In embodiments, the combination comprises BDBM50091347, CPP, and LP-44.

In embodiments, the combination comprises BDBM50091347, NBOH-2C-CN, and 5-CT. In embodiments, the combination comprises BDBM50091347, NBOH-2C-CN, and 5-MeO-DALT. In embodiments, the combination comprises BDBM50091347, NBOH-2C-CN, and 5-MeO-DMT. In embodiments, the combination comprises BDBM50091347, NBOH-2C-CN, and 5-MeO-MiPT In embodiments, the combination comprises BDBM50091347, NBOH-2C-CN, and AGH-192. In embodiments, the combination comprises BDBM50091347, NBOH-2C-CN, and AGH-107. In embodiments, the combination comprises BDBM50091347, NBOH-2C-CN, and AMT. In embodiments, the combination comprises BDBM50091347, NBOH-2C-CN, and aripiprazole. In embodiments, the combination comprises BDBM50091347, NBOH-2C-CN, and AS-19. In embodiments, the combination comprises BDBM50091347, NBOH-2C-CN, and E-55888. In embodiments, the combination comprises BDBM50091347, NBOH-2C-CN, and LSD. In embodiments, the combination comprises BDBM50091347, NBOH-2C-CN, and LP-211. In embodiments, the combination comprises BDBM50091347, NBOH-2C-CN, and LP-44.

In embodiments, the combination comprises BDBM50091347, TCB-2, and 5-CT. In embodiments, the combination comprises BDBM50091347, TCB-2, and 5-MeO-DALT. In embodiments, the combination comprises BDBM50091347, TCB-2, and 5-MeO-DMT In embodiments, the combination comprises BDBM50091347, TCB-2, and 5-MeO-MiPT In embodiments, the combination comprises BDBM50091347, TCB-2, and AGH-192. In embodiments, the combination comprises BDBM50091347, TCB-2, and AGH-107. In embodiments, the combination comprises BDBM50091347, TCB-2, and AMT. In embodiments, the combination comprises BDBM50091347, TCB-2, and aripiprazole. In embodiments, the combination comprises BDBM50091347, TCB-2, and AS-19. In embodiments, the combination comprises BDBM50091347, TCB-2, and E-55888. In embodiments, the combination comprises BDBM50091347, TCB-2, and LSD. In embodiments, the combination comprises BDBM50091347, TCB-2, and LP-211. In embodiments, the combination comprises BDBM50091347, TCB-2, and LP-44.

In embodiments, the combination comprises BDBM50091347, WAY 161503, and 5-CT. In embodiments, the combination comprises BDBM50091347, WAY 161503, and 5-MeO-DALT. In embodiments, the combination comprises BDBM50091347, WAY 161503, and 5-MeO-DMT In embodiments, the combination comprises BDBM50091347, WAY 161503, and 5-MeO-MiPT. In embodiments, the combination comprises BDBM50091347, WAY 161503, and AGH-192. In embodiments, the combination comprises BDBM50091347, WAY 161503, and AGH-107. In embodiments, the combination comprises BDBM50091347, WAY 161503, and AMT. In embodiments, the combination comprises BDBM50091347, WAY 161503, and aripiprazole. In embodiments, the combination comprises BDBM50091347, WAY 161503, and AS-19. In embodiments, the combination comprises BDBM50091347, WAY 161503, and E-55888. In embodiments, the combination comprises BDBM50091347, WAY 161503, and LSD. In embodiments, the combination comprises BDBM50091347, WAY 161503, and LP-211. In embodiments, the combination comprises BDBM50091347, WAY 161503, and LP-44.

In embodiments, the combination comprises clonidine, 1-methylpsilocin, and 5-CT. In embodiments, the combination comprises clonidine, 1-methylpsilocin, and 5-MeO-DALT. In embodiments, the combination comprises clonidine, 1-methylpsilocin, and 5-MeO-DMT In embodiments, the combination comprises clonidine, 1-methylpsilocin, and 5-MeO-MiPT In embodiments, the combination comprises clonidine, 1-methylpsilocin, and AGH-192. In embodiments, the combination comprises clonidine, 1-methylpsilocin, and AGH-107. In embodiments, the combination comprises clonidine, 1-methylpsilocin, and AMT. In embodiments, the combination comprises clonidine, 1-methyl psilocin, and aripiprazole. In embodiments, the combination comprises clonidine, 1-methyl psilocin, and AS-19. In embodiments, the combination comprises clonidine, 1-methylpsilocin, and E-55888. In embodiments, the combination comprises clonidine, 1-methylpsilocin, and LSD. In embodiments, the combination comprises clonidine, 1-methylpsilocin, and LP-211. In embodiments, the combination comprises clonidine, 1-methylpsilocin, and LP-44.

In embodiments, the combination comprises clonidine, 2C-B-FLY, and 5-CT. In embodiments, the combination comprises clonidine, 2C-B-FLY, and 5-MeO-DALT. In embodiments, the combination comprises clonidine, 2C-B-FLY, and 5-MeO-DMT In embodiments, the combination comprises clonidine, 2C-B-FLY, and 5-MeO-MiPT. In embodiments, the combination comprises clonidine, 2C-B-FLY, and AGH-192. In embodiments, the combination comprises clonidine, 2C-B-FLY, and AGH-107. In embodiments, the combination comprises clonidine, 2C-B-FLY, and AMT. In embodiments, the combination comprises clonidine, 2C-B-FLY, and aripiprazole. In embodiments, the combination comprises clonidine, 2C-B-FLY, and AS-19. In embodiments, the combination comprises clonidine, 2C-B-FLY, and E-55888. In embodiments, the combination comprises clonidine, 2C-B-FLY, and LSD. In embodiments, the combination comprises clonidine, 2C-B-FLY, and LP-211. In embodiments, the combination comprises clonidine, 2C-B-FLY, and LP-44.

In embodiments, the combination comprises clonidine, 5-MeO-AMT, and 5-CT. In embodiments, the combination comprises clonidine, 5-MeO-AMT, and 5-MeO-DALT. In embodiments, the combination comprises clonidine, 5-MeO-AMT, and 5-MeO-DMT In embodiments, the combination comprises clonidine, 5-MeO-AMT, and 5-MeO-MiPT In embodiments, the combination comprises clonidine, 5-MeO-AMT, and AGH-192. In embodiments, the combination comprises clonidine, 5-MeO-AMT, and AGH-107. In embodiments, the combination comprises clonidine, 5-MeO-AMT, and AMT. In embodiments, the combination comprises clonidine, 5-MeO-AMT, and aripiprazole. In embodiments, the combination comprises clonidine, 5-MeO-AMT, and AS-19. In embodiments, the combination comprises clonidine, 5-MeO-AMT, and E-55888. In embodiments, the combination comprises clonidine, 5-MeO-AMT, and LSD. In embodiments, the combination comprises clonidine, 5-MeO-AMT, and LP-211. In embodiments, the combination comprises clonidine, 5-MeO-AMT, and LP-44.

In embodiments, the combination comprises clonidine, AL-37350A, and 5-CT. In embodiments, the combination comprises clonidine, AL-37350A, and 5-MeO-DALT. In embodiments, the combination comprises clonidine, AL-37350A, and 5-MeO-DMT. In embodiments, the combination comprises clonidine, AL-37350A, and 5-MeO-MiPT. In embodiments, the combination comprises clonidine, AL-37350A, and AGH-192. In embodiments, the combination comprises clonidine, AL-37350A, and AGH-107. In embodiments, the combination comprises clonidine, AL-37350A, and AMT. In embodiments, the combination comprises clonidine, AL-37350A, and aripiprazole. In embodiments, the combination comprises clonidine, AL-37350A, and AS-19. In embodiments, the combination comprises clonidine, AL-37350A, and E-55888. In embodiments, the combination comprises clonidine, AL-37350A, and LSD. In embodiments, the combination comprises clonidine, AL-37350A, and LP-211. In embodiments, the combination comprises clonidine, AL-37350A, and LP-44.

In embodiments, the combination comprises clonidine, CP 809101, and 5-CT. In embodiments, the combination comprises clonidine, CP 809101, and 5-MeO-DALT. In embodiments, the combination comprises clonidine, CP 809101, and 5-MeO-DMT. In embodiments, the combination comprises clonidine, CP 809101, and 5-MeO-MiPT. In embodiments, the combination comprises clonidine, CP 809101, and AGH-192. In embodiments, the combination comprises clonidine, CP 809101, and AGH-107. In embodiments, the combination comprises clonidine, CP 809101, and AMT. In embodiments, the combination comprises clonidine, CP 809101, and aripiprazole. In embodiments, the combination comprises clonidine, CP 809101, and AS-19. In embodiments, the combination comprises clonidine, CP 809101, and E-55888. In embodiments, the combination comprises clonidine, CP 809101, and LSD. In embodiments, the combination comprises clonidine, CP 809101, and LP-211. In embodiments, the combination comprises clonidine, CP 809101, and LP-44.

In embodiments, the combination comprises clonidine, DALT, and 5-CT. In embodiments, the combination comprises clonidine, DALT, and 5-MeO-DALT. In embodiments, the combination comprises clonidine, DALT, and 5-MeO-DMT In embodiments, the combination comprises clonidine, DALT, and 5-MeO-MiPT. In embodiments, the combination comprises clonidine, DALT, and AGH-192. In embodiments, the combination comprises clonidine, DALT, and AGH-107. In embodiments, the combination comprises clonidine, DALT, and AMT. In embodiments, the combination comprises clonidine, DALT, and aripiprazole. In embodiments, the combination comprises clonidine, DALT, and AS-19. In embodiments, the combination comprises clonidine, DALT, and E-55888. In embodiments, the combination comprises clonidine, DALT, and LSD. In embodiments, the combination comprises clonidine, DALT, and LP-211. In embodiments, the combination comprises clonidine, DALT, and LP-44.

In embodiments, the combination comprises clonidine, DOB, and 5-CT. In embodiments, the combination comprises clonidine, DOB, and 5-MeO-DALT. In embodiments, the combination comprises clonidine, DOB, and 5-MeO-DMT. In embodiments, the combination comprises clonidine, DOB, and 5-MeO-MiPT. In embodiments, the combination comprises clonidine, DOB, and AGH-192. In embodiments, the combination comprises clonidine, DOB, and AGH-107. In embodiments, the combination comprises clonidine, DOB, and AMT. In embodiments, the combination comprises clonidine, DOB, and aripiprazole. In embodiments, the combination comprises clonidine, DOB, and AS-19. In embodiments, the combination comprises clonidine, DOB, and E-55888. In embodiments, the combination comprises clonidine, DOB, and LSD. In embodiments, the combination comprises clonidine, DOB, and LP-211. In embodiments, the combination comprises clonidine, DOB, and LP-44.

In embodiments, the combination comprises clonidine, DOET, and 5-CT. In embodiments, the combination comprises clonidine, DOET, and 5-MeO-DALT. In embodiments, the combination comprises clonidine, DOET, and 5-MeO-DMT In embodiments, the combination comprises clonidine, DOET, and 5-MeO-MiPT. In embodiments, the combination comprises clonidine, DOET, and AGH-192. In embodiments, the combination comprises clonidine, DOET, and AGH-107. In embodiments, the combination comprises clonidine, DOET, and AMT. In embodiments, the combination comprises clonidine, DOET, and aripiprazole. In embodiments, the combination comprises clonidine, DOET, and AS-19. In embodiments, the combination comprises clonidine, DOET, and E-55888. In embodiments, the combination comprises clonidine, DOET, and LSD. In embodiments, the combination comprises clonidine, DOET, and LP-211. In embodiments, the combination comprises clonidine, DOET, and LP-44.

In embodiments, the combination comprises clonidine, DOHx, and 5-CT. In embodiments, the combination comprises clonidine, DOHx, and 5-MeO-DALT. In embodiments, the combination comprises clonidine, DOHx, and 5-MeO-DMT In embodiments, the combination comprises clonidine, DOHx, and 5-MeO-MiPT. In embodiments, the combination comprises clonidine, DOHx, and AGH-192. In embodiments, the combination comprises clonidine, DOHx, and AGH-107. In embodiments, the combination comprises clonidine, DOHx, and AMT. In embodiments, the combination comprises clonidine, DOHx, and aripiprazole. In embodiments, the combination comprises clonidine, DOHx, and AS-19. In embodiments, the combination comprises clonidine, DOHx, and E-55888. In embodiments, the combination comprises clonidine, DOHx, and LSD. In embodiments, the combination comprises clonidine, DOHx, and LP-211. In embodiments, the combination comprises clonidine, DOHx, and LP-44.

In embodiments, the combination comprises clonidine, MEM, and 5-CT. In embodiments, the combination comprises clonidine, MEM, and 5-MeO-DALT. In embodiments, the combination comprises clonidine, MEM, and 5-MeO-DMT. In embodiments, the combination comprises clonidine, MEM, and 5-MeO-MiPT. In embodiments, the combination comprises clonidine, MEM, and AGH-192. In embodiments, the combination comprises clonidine, MEM, and AGH-107. In embodiments, the combination comprises clonidine, MEM, and AMT. In embodiments, the combination comprises clonidine, MEM, and aripiprazole. In embodiments, the combination comprises clonidine, MEM, and AS-19. In embodiments, the combination comprises clonidine, MEM, and E-55888. In embodiments, the combination comprises clonidine, MEM, and LSD. In embodiments, the combination comprises clonidine, MEM, and LP-211. In embodiments, the combination comprises clonidine, MEM, and LP-44.

In embodiments, the combination comprises clonidine, CPP, and 5-CT. In embodiments, the combination comprises clonidine, CPP, and 5-MeO-DALT. In embodiments, the combination comprises clonidine, CPP, and 5-MeO-DMT In embodiments, the combination comprises clonidine, CPP, and 5-MeO-MiPT. In embodiments, the combination comprises clonidine, CPP, and AGH-192. In embodiments, the combination comprises clonidine, CPP, and AGH-107. In embodiments, the combination comprises clonidine, CPP, and AMT. In embodiments, the combination comprises clonidine, CPP, and aripiprazole. In embodiments, the combination comprises clonidine, CPP, and AS-19. In embodiments, the combination comprises clonidine, CPP, and E-55888. In embodiments, the combination comprises clonidine, CPP, and LSD. In embodiments, the combination comprises clonidine, CPP, and LP-211. In embodiments, the combination comprises clonidine, CPP, and LP-44.

In embodiments, the combination comprises clonidine, NBOH-2C-CN, and 5-CT. In embodiments, the combination comprises clonidine, NBOH-2C-CN, and 5-MeO-DALT. In embodiments, the combination comprises clonidine, NBOH-2C-CN, and 5-MeO-DMT In embodiments, the combination comprises clonidine, NBOH-2C-CN, and 5-MeO-MiPT. In embodiments, the combination comprises clonidine, NBOH-2C-CN, and AGH-192. In embodiments, the combination comprises clonidine, NBOH-2C-CN, and AGH-107. In embodiments, the combination comprises clonidine, NBOH-2C-CN, and AMT. In embodiments, the combination comprises clonidine, NBOH-2C-CN, and aripiprazole. In embodiments, the combination comprises clonidine, NBOH-2C-CN, and AS-19. In embodiments, the combination comprises clonidine, NBOH-2C-CN, and E-55888. In embodiments, the combination comprises clonidine, NBOH-2C-CN, and LSD. In embodiments, the combination comprises clonidine, NBOH-2C-CN, and LP-211. In embodiments, the combination comprises clonidine, NBOH-2C-CN, and LP-44.

In embodiments, the combination comprises clonidine, TCB-2, and 5-CT. In embodiments, the combination comprises clonidine, TCB-2, and 5-MeO-DALT. In embodiments, the combination comprises clonidine, TCB-2, and 5-MeO-DMT. In embodiments, the combination comprises clonidine, TCB-2, and 5-MeO-MiPT. In embodiments, the combination comprises clonidine, TCB-2, and AGH-192. In embodiments, the combination comprises clonidine, TCB-2, and AGH-107. In embodiments, the combination comprises clonidine, TCB-2, and AMT. In embodiments, the combination comprises clonidine, TCB-2, and aripiprazole. In embodiments, the combination comprises clonidine, TCB-2, and AS-19. In embodiments, the combination comprises clonidine, TCB-2, and E-55888. In embodiments, the combination comprises clonidine, TCB-2, and LSD. In embodiments, the combination comprises clonidine, TCB-2, and LP-211. In embodiments, the combination comprises clonidine, TCB-2, and LP-44.

In embodiments, the combination comprises clonidine, WAY 161503, and 5-CT. In embodiments, the combination comprises clonidine, WAY 161503, and 5-MeO-DALT. In embodiments, the combination comprises clonidine, WAY 161503, and 5-MeO-DMT. In embodiments, the combination comprises clonidine, WAY 161503, and 5-MeO-MiPT. In embodiments, the combination comprises clonidine, WAY 161503, and AGH-192. In embodiments, the combination comprises clonidine, WAY 161503, and AGH-107. In embodiments, the combination comprises clonidine, WAY 161503, and AMT. In embodiments, the combination comprises clonidine, WAY 161503, and aripiprazole. In embodiments, the combination comprises clonidine, WAY 161503, and AS-19. In embodiments, the combination comprises clonidine, WAY 161503, and E-55888. In embodiments, the combination comprises clonidine, WAY 161503, and LSD. In embodiments, the combination comprises clonidine, WAY 161503, and LP-211. In embodiments, the combination comprises clonidine, WAY 161503, and LP-44.

In embodiments, the combination comprises guanfacine, 1-methylpsilocin, and 5-CT. In embodiments, the combination comprises guanfacine, 1-methylpsilocin, and 5-MeO-DALT. In embodiments, the combination comprises guanfacine, 1-methylpsilocin, and 5-MeO-DMT. In embodiments, the combination comprises guanfacine, 1-methylpsilocin, and 5-MeO-MiPT In embodiments, the combination comprises guanfacine, 1-methylpsilocin, and AGH-192. In embodiments, the combination comprises guanfacine, 1-methylpsilocin, and AGH-107. In embodiments, the combination comprises guanfacine, 1-methylpsilocin, and AMT. In embodiments, the combination comprises guanfacine, 1-methylpsilocin, and aripiprazole. In embodiments, the combination comprises guanfacine, 1-methylpsilocin, and AS-19. In embodiments, the combination comprises guanfacine, 1-methylpsilocin, and E-55888. In embodiments, the combination comprises guanfacine, 1-methyl psilocin, and LSD. In embodiments, the combination comprises guanfacine, 1-methylpsilocin, and LP-211. In embodiments, the combination comprises guanfacine, 1-methylpsilocin, and LP-44.

In embodiments, the combination comprises guanfacine, 2C-B-FLY, and 5-CT. In embodiments, the combination comprises guanfacine, 2C-B-FLY, and 5-MeO-DALT. In embodiments, the combination comprises guanfacine, 2C-B-FLY, and 5-MeO-DMT. In embodiments, the combination comprises guanfacine, 2C-B-FLY, and 5-MeO-MiPT. In embodiments, the combination comprises guanfacine, 2C-B-FLY, and AGH-192. In embodiments, the combination comprises guanfacine, 2C-B-FLY, and AGH-107. In embodiments, the combination comprises guanfacine, 2C-B-FLY, and AMT. In embodiments, the combination comprises guanfacine, 2C-B-FLY, and aripiprazole. In embodiments, the combination comprises guanfacine, 2C-B-FLY, and AS-19. In embodiments, the combination comprises guanfacine, 2C-B-FLY, and E-55888. In embodiments, the combination comprises guanfacine, 2C-B-FLY, and LSD. In embodiments, the combination comprises guanfacine, 2C-B-FLY, and LP-211. In embodiments, the combination comprises guanfacine, 2C-B-FLY, and LP-44.

In embodiments, the combination comprises guanfacine, 5-MeO-AMT, and 5-CT. In embodiments, the combination comprises guanfacine, 5-MeO-AMT, and 5-MeO-DALT. In embodiments, the combination comprises guanfacine, 5-MeO-AMT, and 5-MeO-DMT. In embodiments, the combination comprises guanfacine, 5-MeO-AMT, and 5-MeO-MiPT. In embodiments, the combination comprises guanfacine, 5-MeO-AMT, and AGH-192. In embodiments, the combination comprises guanfacine, 5-MeO-AMT, and AGH-107. In embodiments, the combination comprises guanfacine, 5-MeO-AMT, and AMT. In embodiments, the combination comprises guanfacine, 5-MeO-AMT, and aripiprazole. In embodiments, the combination comprises guanfacine, 5-MeO-AMT, and AS-19. In embodiments, the combination comprises guanfacine, 5-MeO-AMT, and E-55888. In embodiments, the combination comprises guanfacine, 5-MeO-AMT, and LSD. In embodiments, the combination comprises guanfacine, 5-MeO-AMT, and LP-211. In embodiments, the combination comprises guanfacine, 5-MeO-AMT, and LP-44.

In embodiments, the combination comprises guanfacine, AL-37350A, and 5-CT. In embodiments, the combination comprises guanfacine, AL-37350A, and 5-MeO-DALT. In embodiments, the combination comprises guanfacine, AL-37350A, and 5-MeO-DMT In embodiments, the combination comprises guanfacine, AL-37350A, and 5-MeO-MiPT In embodiments, the combination comprises guanfacine, AL-37350A, and AGH-192. In embodiments, the combination comprises guanfacine, AL-37350A, and AGH-107. In embodiments, the combination comprises guanfacine, AL-37350A, and AMT. In embodiments, the combination comprises guanfacine, AL-37350A, and aripiprazole. In embodiments, the combination comprises guanfacine, AL-37350A, and AS-19. In embodiments, the combination comprises guanfacine, AL-37350A, and E-55888. In embodiments, the combination comprises guanfacine, AL-37350A, and LSD. In embodiments, the combination comprises guanfacine, AL-37350A, and LP-211. In embodiments, the combination comprises guanfacine, AL-37350A, and LP-44.

In embodiments, the combination comprises guanfacine, CP 809101, and 5-CT. In embodiments, the combination comprises guanfacine, CP 809101, and 5-MeO-DALT. In embodiments, the combination comprises guanfacine, CP 809101, and 5-MeO-DMT. In embodiments, the combination comprises guanfacine, CP 809101, and 5-MeO-MiPT. In embodiments, the combination comprises guanfacine, CP 809101, and AGH-192. In embodiments, the combination comprises guanfacine, CP 809101, and AGH-107. In embodiments, the combination comprises guanfacine, CP 809101, and AMT. In embodiments, the combination comprises guanfacine, CP 809101, and aripiprazole. In embodiments, the combination comprises guanfacine, CP 809101, and AS-19. In embodiments, the combination comprises guanfacine, CP 809101, and E-55888. In embodiments, the combination comprises guanfacine, CP 809101, and LSD. In embodiments, the combination comprises guanfacine, CP 809101, and LP-211. In embodiments, the combination comprises guanfacine, CP 809101, and LP-44.

In embodiments, the combination comprises guanfacine, DALT, and 5-CT. In embodiments, the combination comprises guanfacine, DALT, and 5-MeO-DALT. In embodiments, the combination comprises guanfacine, DALT, and 5-MeO-DMT In embodiments, the combination comprises guanfacine, DALT, and 5-MeO-MiPT. In embodiments, the combination comprises guanfacine, DALT, and AGH-192. In embodiments, the combination comprises guanfacine, DALT, and AGH-107. In embodiments, the combination comprises guanfacine, DALT, and AMT. In embodiments, the combination comprises guanfacine, DALT, and aripiprazole. In embodiments, the combination comprises guanfacine, DALT, and AS-19. In embodiments, the combination comprises guanfacine, DALT, and E-55888. In embodiments, the combination comprises guanfacine, DALT, and LSD. In embodiments, the combination comprises guanfacine, DALT, and LP-211. In embodiments, the combination comprises guanfacine, DALT, and LP-44.

In embodiments, the combination comprises guanfacine, DOB, and 5-CT. In embodiments, the combination comprises guanfacine, DOB, and 5-MeO-DALT. In embodiments, the combination comprises guanfacine, DOB, and 5-MeO-DMT In embodiments, the combination comprises guanfacine, DOB, and 5-MeO-MiPT. In embodiments, the combination comprises guanfacine, DOB, and AGH-192. In embodiments, the combination comprises guanfacine, DOB, and AGH-107. In embodiments, the combination comprises guanfacine, DOB, and AMT. In embodiments, the combination comprises guanfacine, DOB, and aripiprazole. In embodiments, the combination comprises guanfacine, DOB, and AS-19. In embodiments, the combination comprises guanfacine, DOB, and E-55888. In embodiments, the combination comprises guanfacine, DOB, and LSD. In embodiments, the combination comprises guanfacine, DOB, and LP-211. In embodiments, the combination comprises guanfacine, DOB, and LP-44.

In embodiments, the combination comprises guanfacine, DOET, and 5-CT. In embodiments, the combination comprises guanfacine, DOET, and 5-MeO-DALT. In embodiments, the combination comprises guanfacine, DOET, and 5-MeO-DMT. In embodiments, the combination comprises guanfacine, DOET, and 5-MeO-MiPT. In embodiments, the combination comprises guanfacine, DOET, and AGH-192. In embodiments, the combination comprises guanfacine, DOET, and AGH-107. In embodiments, the combination comprises guanfacine, DOET, and AMT. In embodiments, the combination comprises guanfacine, DOET, and aripiprazole. In embodiments, the combination comprises guanfacine, DOET, and AS-19. In embodiments, the combination comprises guanfacine, DOET, and E-55888. In embodiments, the combination comprises guanfacine, DOET, and LSD. In embodiments, the combination comprises guanfacine, DOET, and LP-211. In embodiments, the combination comprises guanfacine, DOET, and LP-44.

In embodiments, the combination comprises guanfacine, DOHx, and 5-CT. In embodiments, the combination comprises guanfacine, DOHx, and 5-MeO-DALT. In embodiments, the combination comprises guanfacine, DOHx, and 5-MeO-DMT In embodiments, the combination comprises guanfacine, DOHx, and 5-MeO-MiPT. In embodiments, the combination comprises guanfacine, DOHx, and AGH-192. In embodiments, the combination comprises guanfacine, DOHx, and AGH-107. In embodiments, the combination comprises guanfacine, DOHx, and AMT. In embodiments, the combination comprises guanfacine, DOHx, and aripiprazole. In embodiments, the combination comprises guanfacine, DOHx, and AS-19. In embodiments, the combination comprises guanfacine, DOHx, and E-55888. In embodiments, the combination comprises guanfacine, DOHx, and LSD. In embodiments, the combination comprises guanfacine, DOHx, and LP-211. In embodiments, the combination comprises guanfacine, DOHx, and LP-44.

In embodiments, the combination comprises guanfacine, MEM, and 5-CT. In embodiments, the combination comprises guanfacine, MEM, and 5-MeO-DALT. In embodiments, the combination comprises guanfacine, MEM, and 5-MeO-DMT In embodiments, the combination comprises guanfacine, MEM, and 5-MeO-MiPT. In embodiments, the combination comprises guanfacine, MEM, and AGH-192. In embodiments, the combination comprises guanfacine, MEM, and AGH-107. In embodiments, the combination comprises guanfacine, MEM, and AMT. In embodiments, the combination comprises guanfacine, MEM, and aripiprazole. In embodiments, the combination comprises guanfacine, MEM, and AS-19. In embodiments, the combination comprises guanfacine, MEM, and E-55888. In embodiments, the combination comprises guanfacine, MEM, and LSD. In embodiments, the combination comprises guanfacine, MEM, and LP-211. In embodiments, the combination comprises guanfacine, MEM, and LP-44.

In embodiments, the combination comprises guanfacine, CPP, and 5-CT. In embodiments, the combination comprises guanfacine, CPP, and 5-MeO-DALT. In embodiments, the combination comprises guanfacine, CPP, and 5-MeO-DMT In embodiments, the combination comprises guanfacine, CPP, and 5-MeO-MiPT. In embodiments, the combination comprises guanfacine, CPP, and AGH-192. In embodiments, the combination comprises guanfacine, CPP, and AGH-107. In embodiments, the combination comprises guanfacine, CPP, and AMT. In embodiments, the combination comprises guanfacine, CPP, and aripiprazole. In embodiments, the combination comprises guanfacine, CPP, and AS-19. In embodiments, the combination comprises guanfacine, CPP, and E-55888. In embodiments, the combination comprises guanfacine, CPP, and LSD. In embodiments, the combination comprises guanfacine, CPP, and LP-211. In embodiments, the combination comprises guanfacine, CPP, and LP-44.

In embodiments, the combination comprises guanfacine, NBOH-2C-CN, and 5-CT. In embodiments, the combination comprises guanfacine, NBOH-2C-CN, and 5-MeO-DALT. In embodiments, the combination comprises guanfacine, NBOH-2C-CN, and 5-MeO-DMT. In embodiments, the combination comprises guanfacine, NBOH-2C-CN, and 5-MeO-MiPT In embodiments, the combination comprises guanfacine, NBOH-2C-CN, and AGH-192. In embodiments, the combination comprises guanfacine, NBOH-2C-CN, and AGH-107. In embodiments, the combination comprises guanfacine, NBOH-2C-CN, and AMT. In embodiments, the combination comprises guanfacine, NBOH-2C-CN, and aripiprazole. In embodiments, the combination comprises guanfacine, NBOH-2C-CN, and AS-19. In embodiments, the combination comprises guanfacine, NBOH-2C-CN, and E-55888. In embodiments, the combination comprises guanfacine, NBOH-2C-CN, and LSD. In embodiments, the combination comprises guanfacine, NBOH-2C-CN, and LP-211. In embodiments, the combination comprises guanfacine, NBOH-2C-CN, and LP-44.

In embodiments, the combination comprises guanfacine, TCB-2, and 5-CT. In embodiments, the combination comprises guanfacine, TCB-2, and 5-MeO-DALT. In embodiments, the combination comprises guanfacine, TCB-2, and 5-MeO-DMT. In embodiments, the combination comprises guanfacine, TCB-2, and 5-MeO-MiPT. In embodiments, the combination comprises guanfacine, TCB-2, and AGH-192. In embodiments, the combination comprises guanfacine, TCB-2, and AGH-107. In embodiments, the combination comprises guanfacine, TCB-2, and AMT. In embodiments, the combination comprises guanfacine, TCB-2, and aripiprazole. In embodiments, the combination comprises guanfacine, TCB-2, and AS-19. In embodiments, the combination comprises guanfacine, TCB-2, and E-55888. In embodiments, the combination comprises guanfacine, TCB-2, and LSD. In embodiments, the combination comprises guanfacine, TCB-2, and LP-211. In embodiments, the combination comprises guanfacine, TCB-2, and LP-44.

In embodiments, the combination comprises guanfacine, WAY 161503, and 5-CT. In embodiments, the combination comprises guanfacine, WAY 161503, and 5-MeO-DALT. In embodiments, the combination comprises guanfacine, WAY 161503, and 5-MeO-DMT. In embodiments, the combination comprises guanfacine, WAY 161503, and 5-MeO-MiPT. In embodiments, the combination comprises guanfacine, WAY 161503, and AGH-192. In embodiments, the combination comprises guanfacine, WAY 161503, and AGH-107. In embodiments, the combination comprises guanfacine, WAY 161503, and AMT. In embodiments, the combination comprises guanfacine, WAY 161503, and aripiprazole. In embodiments, the combination comprises guanfacine, WAY 161503, and AS-19. In embodiments, the combination comprises guanfacine, WAY 161503, and E-55888. In embodiments, the combination comprises guanfacine, WAY 161503, and LSD. In embodiments, the combination comprises guanfacine, WAY 161503, and LP-211. In embodiments, the combination comprises guanfacine, WAY 161503, and LP-44.

In embodiments, the combination comprises mCPP, 1-methylpsilocin, and 5-CT. In embodiments, the combination comprises mCPP, 1-methylpsilocin, and 5-MeO-DALT. In embodiments, the combination comprises mCPP, 1-methylpsilocin, and 5-MeO-DMT. In embodiments, the combination comprises mCPP, 1-methylpsilocin, and 5-MeO-MiPT. In embodiments, the combination comprises mCPP, 1-methylpsilocin, and AGH-192. In embodiments, the combination comprises mCPP, 1-methylpsilocin, and AGH-107. In embodiments, the combination comprises mCPP, 1-methylpsilocin, and AMT. In embodiments, the combination comprises mCPP, 1-methylpsilocin, and aripiprazole. In embodiments, the combination comprises mCPP, 1-methylpsilocin, and AS-19. In embodiments, the combination comprises mCPP, 1-methylpsilocin, and E-55888. In embodiments, the combination comprises mCPP, 1-methylpsilocin, and LSD. In embodiments, the combination comprises mCPP, 1-methylpsilocin, and LP-211. In embodiments, the combination comprises mCPP, 1-methylpsilocin, and LP-44.

In embodiments, the combination comprises mCPP, 2C-B-FLY, and 5-CT. In embodiments, the combination comprises mCPP, 2C-B-FLY, and 5-MeO-DALT. In embodiments, the combination comprises mCPP, 2C-B-FLY, and 5-MeO-DMT. In embodiments, the combination comprises mCPP, 2C-B-FLY, and 5-MeO-MiPT. In embodiments, the combination comprises mCPP, 2C-B-FLY, and AGH-192. In embodiments, the combination comprises mCPP, 2C-B-FLY, and AGH-107. In embodiments, the combination comprises mCPP, 2C-B-FLY, and AMT. In embodiments, the combination comprises mCPP, 2C-B-FLY, and aripiprazole. In embodiments, the combination comprises mCPP, 2C-B-FLY, and AS-19. In embodiments, the combination comprises mCPP, 2C-B-FLY, and E-55888. In embodiments, the combination comprises mCPP, 2C-B-FLY, and LSD. In embodiments, the combination comprises mCPP, 2C-B-FLY, and LP-211. In embodiments, the combination comprises mCPP, 2C-B-FLY, and LP-44.

In embodiments, the combination comprises mCPP, 5-MeO-AMT, and 5-CT. In embodiments, the combination comprises mCPP, 5-MeO-AMT, and 5-MeO-DALT. In embodiments, the combination comprises mCPP, 5-MeO-AMT, and 5-MeO-DMT. In embodiments, the combination comprises mCPP, 5-MeO-AMT, and 5-MeO-MiPT. In embodiments, the combination comprises mCPP, 5-MeO-AMT, and AGH-192. In embodiments, the combination comprises mCPP, 5-MeO-AMT, and AGH-107. In embodiments, the combination comprises mCPP, 5-MeO-AMT, and AMT. In embodiments, the combination comprises mCPP, 5-MeO-AMT, and aripiprazole. In embodiments, the combination comprises mCPP, 5-MeO-AMT, and AS-19. In embodiments, the combination comprises mCPP, 5-MeO-AMT, and E-55888. In embodiments, the combination comprises mCPP, 5-MeO-AMT, and LSD. In embodiments, the combination comprises mCPP, 5-MeO-AMT, and LP-211. In embodiments, the combination comprises mCPP, 5-MeO-AMT, and LP-44.

In embodiments, the combination comprises mCPP, AL-37350A, and 5-CT. In embodiments, the combination comprises mCPP, AL-37350A, and 5-MeO-DALT. In embodiments, the combination comprises mCPP, AL-37350A, and 5-MeO-DMT In embodiments, the combination comprises mCPP, AL-37350A, and 5-MeO-MiPT. In embodiments, the combination comprises mCPP, AL-37350A, and AGH-192. In embodiments, the combination comprises mCPP, AL-37350A, and AGH-107. In embodiments, the combination comprises mCPP, AL-37350A, and AMT. In embodiments, the combination comprises mCPP, AL-37350A, and aripiprazole. In embodiments, the combination comprises mCPP, AL-37350A, and AS-19. In embodiments, the combination comprises mCPP, AL-37350A, and E-55888. In embodiments, the combination comprises mCPP, AL-37350A, and LSD. In embodiments, the combination comprises mCPP, AL-37350A, and LP-211. In embodiments, the combination comprises mCPP, AL-37350A, and LP-44.

In embodiments, the combination comprises mCPP, CP 809101, and 5-CT. In embodiments, the combination comprises mCPP, CP 809101, and 5-MeO-DALT. In embodiments, the combination comprises mCPP, CP 809101, and 5-MeO-DMT In embodiments, the combination comprises mCPP, CP 809101, and 5-MeO-MiPT. In embodiments, the combination comprises mCPP, CP 809101, and AGH-192. In embodiments, the combination comprises mCPP, CP 809101, and AGH-107. In embodiments, the combination comprises mCPP, CP 809101, and AMT. In embodiments, the combination comprises mCPP, CP 809101, and aripiprazole. In embodiments, the combination comprises mCPP, CP 809101, and AS-19. In embodiments, the combination comprises mCPP, CP 809101, and E-55888. In embodiments, the combination comprises mCPP, CP 809101, and LSD. In embodiments, the combination comprises mCPP, CP 809101, and LP-211. In embodiments, the combination comprises mCPP, CP 809101, and LP-44.

In embodiments, the combination comprises mCPP, DALT, and 5-CT. In embodiments, the combination comprises mCPP, DALT, and 5-MeO-DALT. In embodiments, the combination comprises mCPP, DALT, and 5-MeO-DMT In embodiments, the combination comprises mCPP, DALT, and 5-MeO-MiPT. In embodiments, the combination comprises mCPP, DALT, and AGH-192. In embodiments, the combination comprises mCPP, DALT, and AGH-107. In embodiments, the combination comprises mCPP, DALT, and AMT. In embodiments, the combination comprises mCPP, DALT, and aripiprazole. In embodiments, the combination comprises mCPP, DALT, and AS-19. In embodiments, the combination comprises mCPP, DALT, and E-55888. In embodiments, the combination comprises mCPP, DALT, and LSD. In embodiments, the combination comprises mCPP, DALT, and LP-211. In embodiments, the combination comprises mCPP, DALT, and LP-44.

In embodiments, the combination comprises mCPP, DOB, and 5-CT. In embodiments, the combination comprises mCPP, DOB, and 5-MeO-DALT. In embodiments, the combination comprises mCPP, DOB, and 5-MeO-DMT. In embodiments, the combination comprises mCPP, DOB, and 5-MeO-MiPT. In embodiments, the combination comprises mCPP, DOB, and AGH-192. In embodiments, the combination comprises mCPP, DOB, and AGH-107. In embodiments, the combination comprises mCPP, DOB, and AMT. In embodiments, the combination comprises mCPP, DOB, and aripiprazole. In embodiments, the combination comprises mCPP, DOB, and AS-19. In embodiments, the combination comprises mCPP, DOB, and E-55888. In embodiments, the combination comprises mCPP, DOB, and LSD. In embodiments, the combination comprises mCPP, DOB, and LP-211. In embodiments, the combination comprises mCPP, DOB, and LP-44.

In embodiments, the combination comprises mCPP, DOET, and 5-CT. In embodiments, the combination comprises mCPP, DOET, and 5-MeO-DALT. In embodiments, the combination comprises mCPP, DOET, and 5-MeO-DMT In embodiments, the combination comprises mCPP, DOET, and 5-MeO-MiPT. In embodiments, the combination comprises mCPP, DOET, and AGH-192. In embodiments, the combination comprises mCPP, DOET, and AGH-107. In embodiments, the combination comprises mCPP, DOET, and AMT. In embodiments, the combination comprises mCPP, DOET, and aripiprazole. In embodiments, the combination comprises mCPP, DOET, and AS-19. In embodiments, the combination comprises mCPP, DOET, and E-55888. In embodiments, the combination comprises mCPP, DOET, and LSD. In embodiments, the combination comprises mCPP, DOET, and LP-211. In embodiments, the combination comprises mCPP, DOET, and LP-44.

In embodiments, the combination comprises mCPP, DOHx, and 5-CT. In embodiments, the combination comprises mCPP, DOHx, and 5-MeO-DALT. In embodiments, the combination comprises mCPP, DOHx, and 5-MeO-DMT. In embodiments, the combination comprises mCPP, DOHx, and 5-MeO-MiPT. In embodiments, the combination comprises mCPP, DOHx, and AGH-192. In embodiments, the combination comprises mCPP, DOHx, and AGH-107. In embodiments, the combination comprises mCPP, DOHx, and AMT. In embodiments, the combination comprises mCPP, DOHx, and aripiprazole. In embodiments, the combination comprises mCPP, DOHx, and AS-19. In embodiments, the combination comprises mCPP, DOHx, and E-55888. In embodiments, the combination comprises mCPP, DOHx, and LSD. In embodiments, the combination comprises mCPP, DOHx, and LP-211. In embodiments, the combination comprises mCPP, DOHx, and LP-44.

In embodiments, the combination comprises mCPP, MEM, and 5-CT. In embodiments, the combination comprises mCPP, MEM, and 5-MeO-DALT. In embodiments, the combination comprises mCPP, MEM, and 5-MeO-DMT. In embodiments, the combination comprises mCPP, MEM, and 5-MeO-MiPT. In embodiments, the combination comprises mCPP, MEM, and AGH-192. In embodiments, the combination comprises mCPP, MEM, and AGH-107. In embodiments, the combination comprises mCPP, MEM, and AMT. In embodiments, the combination comprises mCPP, MEM, and aripiprazole. In embodiments, the combination comprises mCPP, MEM, and AS-19. In embodiments, the combination comprises mCPP, MEM, and E-55888. In embodiments, the combination comprises mCPP, MEM, and LSD. In embodiments, the combination comprises mCPP, MEM, and LP-211. In embodiments, the combination comprises mCPP, MEM, and LP-44.

In embodiments, the combination comprises mCPP, CPP, and 5-CT. In embodiments, the combination comprises mCPP, CPP, and 5-MeO-DALT. In embodiments, the combination comprises mCPP, CPP, and 5-MeO-DMT In embodiments, the combination comprises mCPP, CPP, and 5-MeO-MiPT. In embodiments, the combination comprises mCPP, CPP, and AGH-192. In embodiments, the combination comprises mCPP, CPP, and AGH-107. In embodiments, the combination comprises mCPP, CPP, and AMT. In embodiments, the combination comprises mCPP, CPP, and aripiprazole. In embodiments, the combination comprises mCPP, CPP, and AS-19. In embodiments, the combination comprises mCPP, CPP, and E-55888. In embodiments, the combination comprises mCPP, CPP, and LSD. In embodiments, the combination comprises mCPP, CPP, and LP-211. In embodiments, the combination comprises mCPP, CPP, and LP-44.

In embodiments, the combination comprises mCPP, NBOH-2C-CN, and 5-CT. In embodiments, the combination comprises mCPP, NBOH-2C-CN, and 5-MeO-DALT. In embodiments, the combination comprises mCPP, NBOH-2C-CN, and 5-MeO-DMT. In embodiments, the combination comprises mCPP, NBOH-2C-CN, and 5-MeO-MiPT In embodiments, the combination comprises mCPP, NBOH-2C-CN, and AGH-192. In embodiments, the combination comprises mCPP, NBOH-2C-CN, and AGH-107. In embodiments, the combination comprises mCPP, NBOH-2C-CN, and AMT. In embodiments, the combination comprises mCPP, NBOH-2C-CN, and aripiprazole. In embodiments, the combination comprises mCPP, NBOH-2C-CN, and AS-19. In embodiments, the combination comprises mCPP, NBOH-2C-CN, and E-55888. In embodiments, the combination comprises mCPP, NBOH-2C-CN, and LSD. In embodiments, the combination comprises mCPP, NBOH-2C-CN, and LP-211. In embodiments, the combination comprises mCPP, NBOH-2C-CN, and LP-44.

In embodiments, the combination comprises mCPP, TCB-2, and 5-CT. In embodiments, the combination comprises mCPP, TCB-2, and 5-MeO-DALT. In embodiments, the combination comprises mCPP, TCB-2, and 5-MeO-DMT In embodiments, the combination comprises mCPP, TCB-2, and 5-MeO-MiPT. In embodiments, the combination comprises mCPP, TCB-2, and AGH-192. In embodiments, the combination comprises mCPP, TCB-2, and AGH-107. In embodiments, the combination comprises mCPP, TCB-2, and AMT. In embodiments, the combination comprises mCPP, TCB-2, and aripiprazole. In embodiments, the combination comprises mCPP, TCB-2, and AS-19. In embodiments, the combination comprises mCPP, TCB-2, and E-55888. In embodiments, the combination comprises mCPP, TCB-2, and LSD. In embodiments, the combination comprises mCPP, TCB-2, and LP-211. In embodiments, the combination comprises mCPP, TCB-2, and LP-44.

In embodiments, the combination comprises mCPP, WAY 161503, and 5-CT. In embodiments, the combination comprises mCPP, WAY 161503, and 5-MeO-DALT. In embodiments, the combination comprises mCPP, WAY 161503, and 5-MeO-DMT In embodiments, the combination comprises mCPP, WAY 161503, and 5-MeO-MiPT. In embodiments, the combination comprises mCPP, WAY 161503, and AGH-192. In embodiments, the combination comprises mCPP, WAY 161503, and AGH-107. In embodiments, the combination comprises mCPP, WAY 161503, and AMT. In embodiments, the combination comprises mCPP, WAY 161503, and aripiprazole. In embodiments, the combination comprises mCPP, WAY 161503, and AS-19. In embodiments, the combination comprises mCPP, WAY 161503, and E-55888. In embodiments, the combination comprises mCPP, WAY 161503, and LSD. In embodiments, the combination comprises mCPP, WAY 161503, and LP-211. In embodiments, the combination comprises mCPP, WAY 161503, and LP-44.

In embodiments, the combination comprises MDMA, 1-methylpsilocin, and 5-CT. In embodiments, the combination comprises MDMA, 1-methylpsilocin, and 5-MeO-DALT. In embodiments, the combination comprises MDMA, 1-methyl psilocin, and 5-MeO-DMT. In embodiments, the combination comprises MDMA, 1-methylpsilocin, and 5-MeO-MiPT. In embodiments, the combination comprises MDMA, 1-methylpsilocin, and AGH-192. In embodiments, the combination comprises MDMA, 1-methylpsilocin, and AGH-107. In embodiments, the combination comprises MDMA, 1-methylpsilocin, and AMT. In embodiments, the combination comprises MDMA, 1-methylpsilocin, and aripiprazole. In embodiments, the combination comprises MDMA, 1-methylpsilocin, and AS-19. In embodiments, the combination comprises MDMA, 1-methylpsilocin, and E-55888. In embodiments, the combination comprises MDMA, 1-methylpsilocin, and LSD. In embodiments, the combination comprises MDMA, 1-methylpsilocin, and LP-211. In embodiments, the combination comprises MDMA, 1-methyl psilocin, and LP-44.

In embodiments, the combination comprises MDMA, 2C-B-FLY, and 5-CT. In embodiments, the combination comprises MDMA, 2C-B-FLY, and 5-MeO-DALT. In embodiments, the combination comprises MDMA, 2C-B-FLY, and 5-MeO-DMT In embodiments, the combination comprises MDMA, 2C-B-FLY, and 5-MeO-MiPT. In embodiments, the combination comprises MDMA, 2C-B-FLY, and AGH-192. In embodiments, the combination comprises MDMA, 2C-B-FLY, and AGH-107. In embodiments, the combination comprises MDMA, 2C-B-FLY, and AMT. In embodiments, the combination comprises MDMA, 2C-B-FLY, and aripiprazole. In embodiments, the combination comprises MDMA, 2C-B-FLY, and AS-19. In embodiments, the combination comprises MDMA, 2C-B-FLY, and E-55888. In embodiments, the combination comprises MDMA, 2C-B-FLY, and LSD. In embodiments, the combination comprises MDMA, 2C-B-FLY, and LP-211. In embodiments, the combination comprises MDMA, 2C-B-FLY, and LP-44.

In embodiments, the combination comprises MDMA, 5-MeO-AMT, and 5-CT. In embodiments, the combination comprises MDMA, 5-MeO-AMT, and 5-MeO-DALT. In embodiments, the combination comprises MDMA, 5-MeO-AMT, and 5-MeO-DMT. In embodiments, the combination comprises MDMA, 5-MeO-AMT, and 5-MeO-MiPT In embodiments, the combination comprises MDMA, 5-MeO-AMT, and AGH-192. In embodiments, the combination comprises MDMA, 5-MeO-AMT, and AGH-107. In embodiments, the combination comprises MDMA, 5-MeO-AMT, and AMT. In embodiments, the combination comprises MDMA, 5-MeO-AMT, and aripiprazole. In embodiments, the combination comprises MDMA, 5-MeO-AMT, and AS-19. In embodiments, the combination comprises MDMA, 5-MeO-AMT, and E-55888. In embodiments, the combination comprises MDMA, 5-MeO-AMT, and LSD. In embodiments, the combination comprises MDMA, 5-MeO-AMT, and LP-211. In embodiments, the combination comprises MDMA, 5-MeO-AMT, and LP-44.

In embodiments, the combination comprises MDMA, AL-37350A, and 5-CT. In embodiments, the combination comprises MDMA, AL-37350A, and 5-MeO-DALT. In embodiments, the combination comprises MDMA, AL-37350A, and 5-MeO-DMT. In embodiments, the combination comprises MDMA, AL-37350A, and 5-MeO-MiPT. In embodiments, the combination comprises MDMA, AL-37350A, and AGH-192. In embodiments, the combination comprises MDMA, AL-37350A, and AGH-107. In embodiments, the combination comprises MDMA, AL-37350A, and AMT. In embodiments, the combination comprises MDMA, AL-37350A, and aripiprazole. In embodiments, the combination comprises MDMA, AL-37350A, and AS-19. In embodiments, the combination comprises MDMA, AL-37350A, and E-55888. In embodiments, the combination comprises MDMA, AL-37350A, and LSD. In embodiments, the combination comprises MDMA, AL-37350A, and LP-211. In embodiments, the combination comprises MDMA, AL-37350A, and LP-44.

In embodiments, the combination comprises MDMA, CP 809101, and 5-CT. In embodiments, the combination comprises MDMA, CP 809101, and 5-MeO-DALT. In embodiments, the combination comprises MDMA, CP 809101, and 5-MeO-DMT. In embodiments, the combination comprises MDMA, CP 809101, and 5-MeO-MiPT. In embodiments, the combination comprises MDMA, CP 809101, and AGH-192. In embodiments, the combination comprises MDMA, CP 809101, and AGH-107. In embodiments, the combination comprises MDMA, CP 809101, and AMT. In embodiments, the combination comprises MDMA, CP 809101, and aripiprazole. In embodiments, the combination comprises MDMA, CP 809101, and AS-19. In embodiments, the combination comprises MDMA, CP 809101, and E-55888. In embodiments, the combination comprises MDMA, CP 809101, and LSD. In embodiments, the combination comprises MDMA, CP 809101, and LP-211. In embodiments, the combination comprises MDMA, CP 809101, and LP-44.

In embodiments, the combination comprises MDMA, DALT, and 5-CT. In embodiments, the combination comprises MDMA, DALT, and 5-MeO-DALT. In embodiments, the combination comprises MDMA, DALT, and 5-MeO-DMT In embodiments, the combination comprises MDMA, DALT, and 5-MeO-MiPT. In embodiments, the combination comprises MDMA, DALT, and AGH-192. In embodiments, the combination comprises MDMA, DALT, and AGH-107. In embodiments, the combination comprises MDMA, DALT, and AMT. In embodiments, the combination comprises MDMA, DALT, and aripiprazole. In embodiments, the combination comprises MDMA, DALT, and AS-19. In embodiments, the combination comprises MDMA, DALT, and E-55888. In embodiments, the combination comprises MDMA, DALT, and LSD. In embodiments, the combination comprises MDMA, DALT, and LP-211. In embodiments, the combination comprises MDMA, DALT, and LP-44.

In embodiments, the combination comprises MDMA, DOB, and 5-CT. In embodiments, the combination comprises MDMA, DOB, and 5-MeO-DALT. In embodiments, the combination comprises MDMA, DOB, and 5-MeO-DMT In embodiments, the combination comprises MDMA, DOB, and 5-MeO-MiPT. In embodiments, the combination comprises MDMA, DOB, and AGH-192. In embodiments, the combination comprises MDMA, DOB, and AGH-107. In embodiments, the combination comprises MDMA, DOB, and AMT. In embodiments, the combination comprises MDMA, DOB, and aripiprazole. In embodiments, the combination comprises MDMA, DOB, and AS-19. In embodiments, the combination comprises MDMA, DOB, and E-55888. In embodiments, the combination comprises MDMA, DOB, and LSD. In embodiments, the combination comprises MDMA, DOB, and LP-211. In embodiments, the combination comprises MDMA, DOB, and LP-44.

In embodiments, the combination comprises MDMA, DOET, and 5-CT. In embodiments, the combination comprises MDMA, DOET, and 5-MeO-DALT. In embodiments, the combination comprises MDMA, DOET, and 5-MeO-DMT In embodiments, the combination comprises MDMA, DOET, and 5-MeO-MiPT. In embodiments, the combination comprises MDMA, DOET, and AGH-192. In embodiments, the combination comprises MDMA, DOET, and AGH-107. In embodiments, the combination comprises MDMA, DOET, and AMT. In embodiments, the combination comprises MDMA, DOET, and aripiprazole. In embodiments, the combination comprises MDMA, DOET, and AS-19. In embodiments, the combination comprises MDMA, DOET, and E-55888. In embodiments, the combination comprises MDMA, DOET, and LSD. In embodiments, the combination comprises MDMA, DOET, and LP-211. In embodiments, the combination comprises MDMA, DOET, and LP-44.

In embodiments, the combination comprises MDMA, DOHx, and 5-CT. In embodiments, the combination comprises MDMA, DOHx, and 5-MeO-DALT. In embodiments, the combination comprises MDMA, DOHx, and 5-MeO-DMT. In embodiments, the combination comprises MDMA, DOHx, and 5-MeO-MiPT. In embodiments, the combination comprises MDMA, DOHx, and AGH-192. In embodiments, the combination comprises MDMA, DOHx, and AGH-107. In embodiments, the combination comprises MDMA, DOHx, and AMT. In embodiments, the combination comprises MDMA, DOHx, and aripiprazole. In embodiments, the combination comprises MDMA, DOHx, and AS-19. In embodiments, the combination comprises MDMA, DOHx, and E-55888. In embodiments, the combination comprises MDMA, DOHx, and LSD. In embodiments, the combination comprises MDMA, DOHx, and LP-211. In embodiments, the combination comprises MDMA, DOHx, and LP-44.

In embodiments, the combination comprises MDMA, MEM, and 5-CT. In embodiments, the combination comprises MDMA, MEM, and 5-MeO-DALT. In embodiments, the combination comprises MDMA, MEM, and 5-MeO-DMT. In embodiments, the combination comprises MDMA, MEM, and 5-MeO-MiPT. In embodiments, the combination comprises MDMA, MEM, and AGH-192. In embodiments, the combination comprises MDMA, MEM, and AGH-107. In embodiments, the combination comprises MDMA, MEM, and AMT. In embodiments, the combination comprises MDMA, MEM, and aripiprazole. In embodiments, the combination comprises MDMA, MEM, and AS-19. In embodiments, the combination comprises MDMA, MEM, and E-55888. In embodiments, the combination comprises MDMA, MEM, and LSD. In embodiments, the combination comprises MDMA, MEM, and LP-211. In embodiments, the combination comprises MDMA, MEM, and LP-44.

In embodiments, the combination comprises MDMA, CPP, and 5-CT. In embodiments, the combination comprises MDMA, CPP, and 5-MeO-DALT. In embodiments, the combination comprises MDMA, CPP, and 5-MeO-DMT. In embodiments, the combination comprises MDMA, CPP, and 5-MeO-MiPT. In embodiments, the combination comprises MDMA, CPP, and AGH-192. In embodiments, the combination comprises MDMA, CPP, and AGH-107. In embodiments, the combination comprises MDMA, CPP, and AMT. In embodiments, the combination comprises MDMA, CPP, and aripiprazole. In embodiments, the combination comprises MDMA, CPP, and AS-19. In embodiments, the combination comprises MDMA, CPP, and E-55888. In embodiments, the combination comprises MDMA, CPP, and LSD. In embodiments, the combination comprises MDMA, CPP, and LP-211. In embodiments, the combination comprises MDMA, CPP, and LP-44.

In embodiments, the combination comprises MDMA, NBOH-2C-CN, and 5-CT. In embodiments, the combination comprises MDMA, NBOH-2C-CN, and 5-MeO-DALT. In embodiments, the combination comprises MDMA, NBOH-2C-CN, and 5-MeO-DMT In embodiments, the combination comprises MDMA, NBOH-2C-CN, and 5-MeO-MiPT In embodiments, the combination comprises MDMA, NBOH-2C-CN, and AGH-192. In embodiments, the combination comprises MDMA, NBOH-2C-CN, and AGH-107. In embodiments, the combination comprises MDMA, NBOH-2C-CN, and AMT. In embodiments, the combination comprises MDMA, NBOH-2C-CN, and aripiprazole. In embodiments, the combination comprises MDMA, NBOH-2C-CN, and AS-19. In embodiments, the combination comprises MDMA, NBOH-2C-CN, and E-55888. In embodiments, the combination comprises MDMA, NBOH-2C-CN, and LSD. In embodiments, the combination comprises MDMA, NBOH-2C-CN, and LP-211. In embodiments, the combination comprises MDMA, NBOH-2C-CN, and LP-44.

In embodiments, the combination comprises MDMA, TCB-2, and 5-CT. In embodiments, the combination comprises MDMA, TCB-2, and 5-MeO-DALT. In embodiments, the combination comprises MDMA, TCB-2, and 5-MeO-DMT. In embodiments, the combination comprises MDMA, TCB-2, and 5-MeO-MiPT. In embodiments, the combination comprises MDMA, TCB-2, and AGH-192. In embodiments, the combination comprises MDMA, TCB-2, and AGH-107. In embodiments, the combination comprises MDMA, TCB-2, and AMT. In embodiments, the combination comprises MDMA, TCB-2, and aripiprazole. In embodiments, the combination comprises MDMA, TCB-2, and AS-19. In embodiments, the combination comprises MDMA, TCB-2, and E-55888. In embodiments, the combination comprises MDMA, TCB-2, and LSD. In embodiments, the combination comprises MDMA, TCB-2, and LP-211. In embodiments, the combination comprises MDMA, TCB-2, and LP-44.

In embodiments, the combination comprises MDMA, WAY 161503, and 5-CT. In embodiments, the combination comprises MDMA, WAY 161503, and 5-MeO-DALT. In embodiments, the combination comprises MDMA, WAY 161503, and 5-MeO-DMT. In embodiments, the combination comprises MDMA, WAY 161503, and 5-MeO-MiPT In embodiments, the combination comprises MDMA, WAY 161503, and AGH-192. In embodiments, the combination comprises MDMA, WAY 161503, and AGH-107. In embodiments, the combination comprises MDMA, WAY 161503, and AMT. In embodiments, the combination comprises MDMA, WAY 161503, and aripiprazole. In embodiments, the combination comprises MDMA, WAY 161503, and AS-19. In embodiments, the combination comprises MDMA, WAY 161503, and E-55888. In embodiments, the combination comprises MDMA, WAY 161503, and LSD. In embodiments, the combination comprises MDMA, WAY 161503, and LP-211. In embodiments, the combination comprises MDMA, WAY 161503, and LP-44.

In embodiments, the combination comprises memantine, 1-methylpsilocin, and 5-CT. In embodiments, the combination comprises memantine, 1-methylpsilocin, and 5-MeO-DALT. In embodiments, the combination comprises memantine, 1-methylpsilocin, and 5-MeO-DMT In embodiments, the combination comprises memantine, 1-methylpsilocin, and 5-MeO-MiPT. In embodiments, the combination comprises memantine, 1-methylpsilocin, and AGH-192. In embodiments, the combination comprises memantine, 1-methylpsilocin, and AGH-107. In embodiments, the combination comprises memantine, 1-methylpsilocin, and AMT. In embodiments, the combination comprises memantine, 1-methylpsilocin, and aripiprazole. In embodiments, the combination comprises memantine, 1-methylpsilocin, and AS-19. In embodiments, the combination comprises memantine, 1-methylpsilocin, and E-55888. In embodiments, the combination comprises memantine, 1-methylpsilocin, and LSD. In embodiments, the combination comprises memantine, 1-methylpsilocin, and LP-211. In embodiments, the combination comprises memantine, 1-methylpsilocin, and LP-44.

In embodiments, the combination comprises memantine, 2C-B-FLY, and 5-CT. In embodiments, the combination comprises memantine, 2C-B-FLY, and 5-MeO-DALT. In embodiments, the combination comprises memantine, 2C-B-FLY, and 5-MeO-DMT. In embodiments, the combination comprises memantine, 2C-B-FLY, and 5-MeO-MiPT. In embodiments, the combination comprises memantine, 2C-B-FLY, and AGH-192. In embodiments, the combination comprises memantine, 2C-B-FLY, and AGH-107. In embodiments, the combination comprises memantine, 2C-B-FLY, and AMT. In embodiments, the combination comprises memantine, 2C-B-FLY, and aripiprazole. In embodiments, the combination comprises memantine, 2C-B-FLY, and AS-19. In embodiments, the combination comprises memantine, 2C-B-FLY, and E-55888. In embodiments, the combination comprises memantine, 2C-B-FLY, and LSD. In embodiments, the combination comprises memantine, 2C-B-FLY, and LP-211. In embodiments, the combination comprises memantine, 2C-B-FLY, and LP-44.

In embodiments, the combination comprises memantine, 5-MeO-AMT, and 5-CT. In embodiments, the combination comprises memantine, 5-MeO-AMT, and 5-MeO-DALT. In embodiments, the combination comprises memantine, 5-MeO-AMT, and 5-MeO-DMT. In embodiments, the combination comprises memantine, 5-MeO-AMT, and 5-MeO-MiPT In embodiments, the combination comprises memantine, 5-MeO-AMT, and AGH-192. In embodiments, the combination comprises memantine, 5-MeO-AMT, and AGH-107. In embodiments, the combination comprises memantine, 5-MeO-AMT, and AMT. In embodiments, the combination comprises memantine, 5-MeO-AMT, and aripiprazole. In embodiments, the combination comprises memantine, 5-MeO-AMT, and AS-19. In embodiments, the combination comprises memantine, 5-MeO-AMT, and E-55888. In embodiments, the combination comprises memantine, 5-MeO-AMT, and LSD. In embodiments, the combination comprises memantine, 5-MeO-AMT, and LP-211. In embodiments, the combination comprises memantine, 5-MeO-AMT, and LP-44.

In embodiments, the combination comprises memantine, AL-37350A, and 5-CT. In embodiments, the combination comprises memantine, AL-37350A, and 5-MeO-DALT. In embodiments, the combination comprises memantine, AL-37350A, and 5-MeO-DMT In embodiments, the combination comprises memantine, AL-37350A, and 5-MeO-MiPT In embodiments, the combination comprises memantine, AL-37350A, and AGH-192. In embodiments, the combination comprises memantine, AL-37350A, and AGH-107. In embodiments, the combination comprises memantine, AL-37350A, and AMT. In embodiments, the combination comprises memantine, AL-37350A, and aripiprazole. In embodiments, the combination comprises memantine, AL-37350A, and AS-19. In embodiments, the combination comprises memantine, AL-37350A, and E-55888. In embodiments, the combination comprises memantine, AL-37350A, and LSD. In embodiments, the combination comprises memantine, AL-37350A, and LP-211. In embodiments, the combination comprises memantine, AL-37350A, and LP-44.

In embodiments, the combination comprises memantine, CP 809101, and 5-CT. In embodiments, the combination comprises memantine, CP 809101, and 5-MeO-DALT. In embodiments, the combination comprises memantine, CP 809101, and 5-MeO-DMT. In embodiments, the combination comprises memantine, CP 809101, and 5-MeO-MiPT. In embodiments, the combination comprises memantine, CP 809101, and AGH-192. In embodiments, the combination comprises memantine, CP 809101, and AGH-107. In embodiments, the combination comprises memantine, CP 809101, and AMT. In embodiments, the combination comprises memantine, CP 809101, and aripiprazole. In embodiments, the combination comprises memantine, CP 809101, and AS-19. In embodiments, the combination comprises memantine, CP 809101, and E-55888. In embodiments, the combination comprises memantine, CP 809101, and LSD. In embodiments, the combination comprises memantine, CP 809101, and LP-211. In embodiments, the combination comprises memantine, CP 809101, and LP-44.

In embodiments, the combination comprises memantine, DALT, and 5-CT. In embodiments, the combination comprises memantine, DALT, and 5-MeO-DALT. In embodiments, the combination comprises memantine, DALT, and 5-MeO-DMT In embodiments, the combination comprises memantine, DALT, and 5-MeO-MiPT. In embodiments, the combination comprises memantine, DALT, and AGH-192. In embodiments, the combination comprises memantine, DALT, and AGH-107. In embodiments, the combination comprises memantine, DALT, and AMT. In embodiments, the combination comprises memantine, DALT, and aripiprazole. In embodiments, the combination comprises memantine, DALT, and AS-19. In embodiments, the combination comprises memantine, DALT, and E-55888. In embodiments, the combination comprises memantine, DALT, and LSD. In embodiments, the combination comprises memantine, DALT, and LP-211. In embodiments, the combination comprises memantine, DALT, and LP-44.

In embodiments, the combination comprises memantine, DOB, and 5-CT. In embodiments, the combination comprises memantine, DOB, and 5-MeO-DALT. In embodiments, the combination comprises memantine, DOB, and 5-MeO-DMT In embodiments, the combination comprises memantine, DOB, and 5-MeO-MiPT. In embodiments, the combination comprises memantine, DOB, and AGH-192. In embodiments, the combination comprises memantine, DOB, and AGH-107. In embodiments, the combination comprises memantine, DOB, and AMT. In embodiments, the combination comprises memantine, DOB, and aripiprazole. In embodiments, the combination comprises memantine, DOB, and AS-19. In embodiments, the combination comprises memantine, DOB, and E-55888. In embodiments, the combination comprises memantine, DOB, and LSD. In embodiments, the combination comprises memantine, DOB, and LP-211. In embodiments, the combination comprises memantine, DOB, and LP-44.

In embodiments, the combination comprises memantine, DOET, and 5-CT. In embodiments, the combination comprises memantine, DOET, and 5-MeO-DALT. In embodiments, the combination comprises memantine, DOET, and 5-MeO-DMT. In embodiments, the combination comprises memantine, DOET, and 5-MeO-MiPT. In embodiments, the combination comprises memantine, DOET, and AGH-192. In embodiments, the combination comprises memantine, DOET, and AGH-107. In embodiments, the combination comprises memantine, DOET, and AMT. In embodiments, the combination comprises memantine, DOET, and aripiprazole. In embodiments, the combination comprises memantine, DOET, and AS-19. In embodiments, the combination comprises memantine, DOET, and E-55888. In embodiments, the combination comprises memantine, DOET, and LSD. In embodiments, the combination comprises memantine, DOET, and LP-211. In embodiments, the combination comprises memantine, DOET, and LP-44.

In embodiments, the combination comprises memantine, DOHx, and 5-CT. In embodiments, the combination comprises memantine, DOHx, and 5-MeO-DALT. In embodiments, the combination comprises memantine, DOHx, and 5-MeO-DMT In embodiments, the combination comprises memantine, DOHx, and 5-MeO-MiPT. In embodiments, the combination comprises memantine, DOHx, and AGH-192. In embodiments, the combination comprises memantine, DOHx, and AGH-107. In embodiments, the combination comprises memantine, DOHx, and AMT. In embodiments, the combination comprises memantine, DOHx, and aripiprazole. In embodiments, the combination comprises memantine, DOHx, and AS-19. In embodiments, the combination comprises memantine, DOHx, and E-55888. In embodiments, the combination comprises memantine, DOHx, and LSD. In embodiments, the combination comprises memantine, DOHx, and LP-211. In embodiments, the combination comprises memantine, DOHx, and LP-44.

In embodiments, the combination comprises memantine, MEM, and 5-CT. In embodiments, the combination comprises memantine, MEM, and 5-MeO-DALT. In embodiments, the combination comprises memantine, MEM, and 5-MeO-DMT. In embodiments, the combination comprises memantine, MEM, and 5-MeO-MiPT. In embodiments, the combination comprises memantine, MEM, and AGH-192. In embodiments, the combination comprises memantine, MEM, and AGH-107. In embodiments, the combination comprises memantine, MEM, and AMT. In embodiments, the combination comprises memantine, MEM, and aripiprazole. In embodiments, the combination comprises memantine, MEM, and AS-19. In embodiments, the combination comprises memantine, MEM, and E-55888. In embodiments, the combination comprises memantine, MEM, and LSD. In embodiments, the combination comprises memantine, MEM, and LP-211. In embodiments, the combination comprises memantine, MEM, and LP-44.

In embodiments, the combination comprises memantine, CPP, and 5-CT. In embodiments, the combination comprises memantine, CPP, and 5-MeO-DALT. In embodiments, the combination comprises memantine, CPP, and 5-MeO-DMT In embodiments, the combination comprises memantine, CPP, and 5-MeO-MiPT. In embodiments, the combination comprises memantine, CPP, and AGH-192. In embodiments, the combination comprises memantine, CPP, and AGH-107. In embodiments, the combination comprises memantine, CPP, and AMT. In embodiments, the combination comprises memantine, CPP, and aripiprazole. In embodiments, the combination comprises memantine, CPP, and AS-19. In embodiments, the combination comprises memantine, CPP, and E-55888. In embodiments, the combination comprises memantine, CPP, and LSD. In embodiments, the combination comprises memantine, CPP, and LP-211. In embodiments, the combination comprises memantine, CPP, and LP-44.

In embodiments, the combination comprises memantine, NBOH-2C-CN, and 5-CT. In embodiments, the combination comprises memantine, NBOH-2C-CN, and 5-MeO-DALT. In embodiments, the combination comprises memantine, NBOH-2C-CN, and 5-MeO-DMT. In embodiments, the combination comprises memantine, NBOH-2C-CN, and 5-MeO-MiPT In embodiments, the combination comprises memantine, NBOH-2C-CN, and AGH-192. In embodiments, the combination comprises memantine, NBOH-2C-CN, and AGH-107. In embodiments, the combination comprises memantine, NBOH-2C-CN, and AMT. In embodiments, the combination comprises memantine, NBOH-2C-CN, and aripiprazole. In embodiments, the combination comprises memantine, NBOH-2C-CN, and AS-19. In embodiments, the combination comprises memantine, NBOH-2C-CN, and E-55888. In embodiments, the combination comprises memantine, NBOH-2C-CN, and LSD. In embodiments, the combination comprises memantine, NBOH-2C-CN, and LP-211. In embodiments, the combination comprises memantine, NBOH-2C-CN, and LP-44.

In embodiments, the combination comprises memantine, TCB-2, and 5-CT. In embodiments, the combination comprises memantine, TCB-2, and 5-MeO-DALT. In embodiments, the combination comprises memantine, TCB-2, and 5-MeO-DMT In embodiments, the combination comprises memantine, TCB-2, and 5-MeO-MiPT. In embodiments, the combination comprises memantine, TCB-2, and AGH-192. In embodiments, the combination comprises memantine, TCB-2, and AGH-107. In embodiments, the combination comprises memantine, TCB-2, and AMT. In embodiments, the combination comprises memantine, TCB-2, and aripiprazole. In embodiments, the combination comprises memantine, TCB-2, and AS-19. In embodiments, the combination comprises memantine, TCB-2, and E-55888. In embodiments, the combination comprises memantine, TCB-2, and LSD. In embodiments, the combination comprises memantine, TCB-2, and LP-211. In embodiments, the combination comprises memantine, TCB-2, and LP-44.

In embodiments, the combination comprises memantine, WAY 161503, and 5-CT. In embodiments, the combination comprises memantine, WAY 161503, and 5-MeO-DALT. In embodiments, the combination comprises memantine, WAY 161503, and 5-MeO-DMT In embodiments, the combination comprises memantine, WAY 161503, and 5-MeO-MiPT. In embodiments, the combination comprises memantine, WAY 161503, and AGH-192. In embodiments, the combination comprises memantine, WAY 161503, and AGH-107. In embodiments, the combination comprises memantine, WAY 161503, and AMT. In embodiments, the combination comprises memantine, WAY 161503, and aripiprazole. In embodiments, the combination comprises memantine, WAY 161503, and AS-19. In embodiments, the combination comprises memantine, WAY 161503, and E-55888. In embodiments, the combination comprises memantine, WAY 161503, and LSD. In embodiments, the combination comprises memantine, WAY 161503, and LP-211. In embodiments, the combination comprises memantine, WAY 161503, and LP-44.

In embodiments, the combination comprises moxonidine, 1-methylpsilocin, and 5-CT. In embodiments, the combination comprises moxonidine, 1-methylpsilocin, and 5-MeO-DALT. In embodiments, the combination comprises moxonidine, 1-methylpsilocin, and 5-MeO-DMT In embodiments, the combination comprises moxonidine, 1-methylpsilocin, and 5-MeO-MiPT In embodiments, the combination comprises moxonidine, 1-methylpsilocin, and AGH-192. In embodiments, the combination comprises moxonidine, 1-methylpsilocin, and AGH-107. In embodiments, the combination comprises moxonidine, 1-methylpsilocin, and AMT. In embodiments, the combination comprises moxonidine, 1-methylpsilocin, and aripiprazole. In embodiments, the combination comprises moxonidine, 1-methylpsilocin, and AS-19. In embodiments, the combination comprises moxonidine, 1-methylpsilocin, and E-55888. In embodiments, the combination comprises moxonidine, 1-methylpsilocin, and LSD. In embodiments, the combination comprises moxonidine, 1-methylpsilocin, and LP-211. In embodiments, the combination comprises moxonidine, 1-methyl psilocin, and LP-44.

In embodiments, the combination comprises moxonidine, 2C-B-FLY, and 5-CT. In embodiments, the combination comprises moxonidine, 2C-B-FLY, and 5-MeO-DALT. In embodiments, the combination comprises moxonidine, 2C-B-FLY, and 5-MeO-DMT In embodiments, the combination comprises moxonidine, 2C-B-FLY, and 5-MeO-MiPT In embodiments, the combination comprises moxonidine, 2C-B-FLY, and AGH-192. In embodiments, the combination comprises moxonidine, 2C-B-FLY, and AGH-107. In embodiments, the combination comprises moxonidine, 2C-B-FLY, and AMT. In embodiments, the combination comprises moxonidine, 2C-B-FLY, and aripiprazole. In embodiments, the combination comprises moxonidine, 2C-B-FLY, and AS-19. In embodiments, the combination comprises moxonidine, 2C-B-FLY, and E-55888. In embodiments, the combination comprises moxonidine, 2C-B-FLY, and LSD. In embodiments, the combination comprises moxonidine, 2C-B-FLY, and LP-211. In embodiments, the combination comprises moxonidine, 2C-B-FLY, and LP-44.

In embodiments, the combination comprises moxonidine, 5-MeO-AMT, and 5-CT. In embodiments, the combination comprises moxonidine, 5-MeO-AMT, and 5-MeO-DALT. In embodiments, the combination comprises moxonidine, 5-MeO-AMT, and 5-MeO-DMT. In embodiments, the combination comprises moxonidine, 5-MeO-AMT, and 5-MeO-MiPT. In embodiments, the combination comprises moxonidine, 5-MeO-AMT, and AGH-192. In embodiments, the combination comprises moxonidine, 5-MeO-AMT, and AGH-107. In embodiments, the combination comprises moxonidine, 5-MeO-AMT, and AMT. In embodiments, the combination comprises moxonidine, 5-MeO-AMT, and aripiprazole. In embodiments, the combination comprises moxonidine, 5-MeO-AMT, and AS-19. In embodiments, the combination comprises moxonidine, 5-MeO-AMT, and E-55888. In embodiments, the combination comprises moxonidine, 5-MeO-AMT, and LSD. In embodiments, the combination comprises moxonidine, 5-MeO-AMT, and LP-211. In embodiments, the combination comprises moxonidine, 5-MeO-AMT, and LP-44.

In embodiments, the combination comprises moxonidine, AL-37350A, and 5-CT. In embodiments, the combination comprises moxonidine, AL-37350A, and 5-MeO-DALT. In embodiments, the combination comprises moxonidine, AL-37350A, and 5-MeO-DMT. In embodiments, the combination comprises moxonidine, AL-37350A, and 5-MeO-MiPT In embodiments, the combination comprises moxonidine, AL-37350A, and AGH-192. In embodiments, the combination comprises moxonidine, AL-37350A, and AGH-107. In embodiments, the combination comprises moxonidine, AL-37350A, and AMT. In embodiments, the combination comprises moxonidine, AL-37350A, and aripiprazole. In embodiments, the combination comprises moxonidine, AL-37350A, and AS-19. In embodiments, the combination comprises moxonidine, AL-37350A, and E-55888. In embodiments, the combination comprises moxonidine, AL-37350A, and LSD. In embodiments, the combination comprises moxonidine, AL-37350A, and LP-211. In embodiments, the combination comprises moxonidine, AL-37350A, and LP-44.

In embodiments, the combination comprises moxonidine, CP 809101, and 5-CT. In embodiments, the combination comprises moxonidine, CP 809101, and 5-MeO-DALT. In embodiments, the combination comprises moxonidine, CP 809101, and 5-MeO-DMT In embodiments, the combination comprises moxonidine, CP 809101, and 5-MeO-MiPT In embodiments, the combination comprises moxonidine, CP 809101, and AGH-192. In embodiments, the combination comprises moxonidine, CP 809101, and AGH-107. In embodiments, the combination comprises moxonidine, CP 809101, and AMT. In embodiments, the combination comprises moxonidine, CP 809101, and aripiprazole. In embodiments, the combination comprises moxonidine, CP 809101, and AS-19. In embodiments, the combination comprises moxonidine, CP 809101, and E-55888. In embodiments, the combination comprises moxonidine, CP 809101, and LSD. In embodiments, the combination comprises moxonidine, CP 809101, and LP-211. In embodiments, the combination comprises moxonidine, CP 809101, and LP-44.

In embodiments, the combination comprises moxonidine, DALT, and 5-CT. In embodiments, the combination comprises moxonidine, DALT, and 5-MeO-DALT. In embodiments, the combination comprises moxonidine, DALT, and 5-MeO-DMT In embodiments, the combination comprises moxonidine, DALT, and 5-MeO-MiPT. In embodiments, the combination comprises moxonidine, DALT, and AGH-192. In embodiments, the combination comprises moxonidine, DALT, and AGH-107. In embodiments, the combination comprises moxonidine, DALT, and AMT. In embodiments, the combination comprises moxonidine, DALT, and aripiprazole. In embodiments, the combination comprises moxonidine, DALT, and AS-19. In embodiments, the combination comprises moxonidine, DALT, and E-55888. In embodiments, the combination comprises moxonidine, DALT, and LSD. In embodiments, the combination comprises moxonidine, DALT, and LP-211. In embodiments, the combination comprises moxonidine, DALT, and LP-44.

In embodiments, the combination comprises moxonidine, DOB, and 5-CT. In embodiments, the combination comprises moxonidine, DOB, and 5-MeO-DALT. In embodiments, the combination comprises moxonidine, DOB, and 5-MeO-DMT. In embodiments, the combination comprises moxonidine, DOB, and 5-MeO-MiPT. In embodiments, the combination comprises moxonidine, DOB, and AGH-192. In embodiments, the combination comprises moxonidine, DOB, and AGH-107. In embodiments, the combination comprises moxonidine, DOB, and AMT. In embodiments, the combination comprises moxonidine, DOB, and aripiprazole. In embodiments, the combination comprises moxonidine, DOB, and AS-19. In embodiments, the combination comprises moxonidine, DOB, and E-55888. In embodiments, the combination comprises moxonidine, DOB, and LSD. In embodiments, the combination comprises moxonidine, DOB, and LP-211. In embodiments, the combination comprises moxonidine, DOB, and LP-44.

In embodiments, the combination comprises moxonidine, DOET, and 5-CT. In embodiments, the combination comprises moxonidine, DOET, and 5-MeO-DALT. In embodiments, the combination comprises moxonidine, DOET, and 5-MeO-DMT. In embodiments, the combination comprises moxonidine, DOET, and 5-MeO-MiPT. In embodiments, the combination comprises moxonidine, DOET, and AGH-192. In embodiments, the combination comprises moxonidine, DOET, and AGH-107. In embodiments, the combination comprises moxonidine, DOET, and AMT. In embodiments, the combination comprises moxonidine, DOET, and aripiprazole. In embodiments, the combination comprises moxonidine, DOET, and AS-19. In embodiments, the combination comprises moxonidine, DOET, and E-55888. In embodiments, the combination comprises moxonidine, DOET, and LSD. In embodiments, the combination comprises moxonidine, DOET, and LP-211. In embodiments, the combination comprises moxonidine, DOET, and LP-44.

In embodiments, the combination comprises moxonidine, DOHx, and 5-CT. In embodiments, the combination comprises moxonidine, DOHx, and 5-MeO-DALT. In embodiments, the combination comprises moxonidine, DOHx, and 5-MeO-DMT In embodiments, the combination comprises moxonidine, DOHx, and 5-MeO-MiPT. In embodiments, the combination comprises moxonidine, DOHx, and AGH-192. In embodiments, the combination comprises moxonidine, DOHx, and AGH-107. In embodiments, the combination comprises moxonidine, DOHx, and AMT. In embodiments, the combination comprises moxonidine, DOHx, and aripiprazole. In embodiments, the combination comprises moxonidine, DOHx, and AS-19. In embodiments, the combination comprises moxonidine, DOHx, and E-55888. In embodiments, the combination comprises moxonidine, DOHx, and LSD. In embodiments, the combination comprises moxonidine, DOHx, and LP-211. In embodiments, the combination comprises moxonidine, DOHx, and LP-44.

In embodiments, the combination comprises moxonidine, MEM, and 5-CT. In embodiments, the combination comprises moxonidine, MEM, and 5-MeO-DALT. In embodiments, the combination comprises moxonidine, MEM, and 5-MeO-DMT. In embodiments, the combination comprises moxonidine, MEM, and 5-MeO-MiPT. In embodiments, the combination comprises moxonidine, MEM, and AGH-192. In embodiments, the combination comprises moxonidine, MEM, and AGH-107. In embodiments, the combination comprises moxonidine, MEM, and AMT. In embodiments, the combination comprises moxonidine, MEM, and aripiprazole. In embodiments, the combination comprises moxonidine, MEM, and AS-19. In embodiments, the combination comprises moxonidine, MEM, and E-55888. In embodiments, the combination comprises moxonidine, MEM, and LSD. In embodiments, the combination comprises moxonidine, MEM, and LP-211. In embodiments, the combination comprises moxonidine, MEM, and LP-44.

In embodiments, the combination comprises moxonidine, CPP, and 5-CT. In embodiments, the combination comprises moxonidine, CPP, and 5-MeO-DALT. In embodiments, the combination comprises moxonidine, CPP, and 5-MeO-DMT In embodiments, the combination comprises moxonidine, CPP, and 5-MeO-MiPT. In embodiments, the combination comprises moxonidine, CPP, and AGH-192. In embodiments, the combination comprises moxonidine, CPP, and AGH-107. In embodiments, the combination comprises moxonidine, CPP, and AMT. In embodiments, the combination comprises moxonidine, CPP, and aripiprazole. In embodiments, the combination comprises moxonidine, CPP, and AS-19. In embodiments, the combination comprises moxonidine, CPP, and E-55888. In embodiments, the combination comprises moxonidine, CPP, and LSD. In embodiments, the combination comprises moxonidine, CPP, and LP-211. In embodiments, the combination comprises moxonidine, CPP, and LP-44.

In embodiments, the combination comprises moxonidine, NBOH-2C-CN, and 5-CT. In embodiments, the combination comprises moxonidine, NBOH-2C-CN, and 5-MeO-DALT. In embodiments, the combination comprises moxonidine, NBOH-2C-CN, and 5-MeO-DMT. In embodiments, the combination comprises moxonidine, NBOH-2C-CN, and 5-MeO-MiPT. In embodiments, the combination comprises moxonidine, NBOH-2C-CN, and AGH-192. In embodiments, the combination comprises moxonidine, NBOH-2C-CN, and AGH-107. In embodiments, the combination comprises moxonidine, NBOH-2C-CN, and AMT. In embodiments, the combination comprises moxonidine, NBOH-2C-CN, and aripiprazole. In embodiments, the combination comprises moxonidine, NBOH-2C-CN, and AS-19. In embodiments, the combination comprises moxonidine, NBOH-2C-CN, and E-55888. In embodiments, the combination comprises moxonidine, NBOH-2C-CN, and LSD. In embodiments, the combination comprises moxonidine, NBOH-2C-CN, and LP-211. In embodiments, the combination comprises moxonidine, NBOH-2C-CN, and LP-44.

In embodiments, the combination comprises moxonidine, TCB-2, and 5-CT. In embodiments, the combination comprises moxonidine, TCB-2, and 5-MeO-DALT. In embodiments, the combination comprises moxonidine, TCB-2, and 5-MeO-DMT. In embodiments, the combination comprises moxonidine, TCB-2, and 5-MeO-MiPT. In embodiments, the combination comprises moxonidine, TCB-2, and AGH-192. In embodiments, the combination comprises moxonidine, TCB-2, and AGH-107. In embodiments, the combination comprises moxonidine, TCB-2, and AMT. In embodiments, the combination comprises moxonidine, TCB-2, and aripiprazole. In embodiments, the combination comprises moxonidine, TCB-2, and AS-19. In embodiments, the combination comprises moxonidine, TCB-2, and E-55888. In embodiments, the combination comprises moxonidine, TCB-2, and LSD. In embodiments, the combination comprises moxonidine, TCB-2, and LP-211. In embodiments, the combination comprises moxonidine, TCB-2, and LP-44.

In embodiments, the combination comprises moxonidine, WAY 161503, and 5-CT. In embodiments, the combination comprises moxonidine, WAY 161503, and 5-MeO-DALT. In embodiments, the combination comprises moxonidine, WAY 161503, and 5-MeO-DMT. In embodiments, the combination comprises moxonidine, WAY 161503, and 5-MeO-MiPT. In embodiments, the combination comprises moxonidine, WAY 161503, and AGH-192. In embodiments, the combination comprises moxonidine, WAY 161503, and AGH-107. In embodiments, the combination comprises moxonidine, WAY 161503, and AMT. In embodiments, the combination comprises moxonidine, WAY 161503, and aripiprazole. In embodiments, the combination comprises moxonidine, WAY 161503, and AS-19. In embodiments, the combination comprises moxonidine, WAY 161503, and E-55888. In embodiments, the combination comprises moxonidine, WAY 161503, and LSD. In embodiments, the combination comprises moxonidine, WAY 161503, and LP-211. In embodiments, the combination comprises moxonidine, WAY 161503, and LP-44.

In embodiments, the combination comprises naphazoline, 1-methylpsilocin, and 5-CT. In embodiments, the combination comprises naphazoline, 1-methylpsilocin, and 5-MeO-DALT. In embodiments, the combination comprises naphazoline, 1-methylpsilocin, and 5-MeO-DMT In embodiments, the combination comprises naphazoline, 1-methylpsilocin, and 5-MeO-MiPT. In embodiments, the combination comprises naphazoline, 1-methylpsilocin, and AGH-192. In embodiments, the combination comprises naphazoline, 1-methylpsilocin, and AGH-107. In embodiments, the combination comprises naphazoline, 1-methylpsilocin, and AMT. In embodiments, the combination comprises naphazoline, 1-methylpsilocin, and aripiprazole. In embodiments, the combination comprises naphazoline, 1-methylpsilocin, and AS-19. In embodiments, the combination comprises naphazoline, 1-methylpsilocin, and E-55888. In embodiments, the combination comprises naphazoline, 1-methylpsilocin, and LSD. In embodiments, the combination comprises naphazoline, 1-methylpsilocin, and LP-211. In embodiments, the combination comprises naphazoline, 1-methylpsilocin, and LP-44.

In embodiments, the combination comprises naphazoline, 2C-B-FLY, and 5-CT. In embodiments, the combination comprises naphazoline, 2C-B-FLY, and 5-MeO-DALT. In embodiments, the combination comprises naphazoline, 2C-B-FLY, and 5-MeO-DMT. In embodiments, the combination comprises naphazoline, 2C-B-FLY, and 5-MeO-MiPT In embodiments, the combination comprises naphazoline, 2C-B-FLY, and AGH-192. In embodiments, the combination comprises naphazoline, 2C-B-FLY, and AGH-107. In embodiments, the combination comprises naphazoline, 2C-B-FLY, and AMT. In embodiments, the combination comprises naphazoline, 2C-B-FLY, and aripiprazole. In embodiments, the combination comprises naphazoline, 2C-B-FLY, and AS-19. In embodiments, the combination comprises naphazoline, 2C-B-FLY, and E-55888. In embodiments, the combination comprises naphazoline, 2C-B-FLY, and LSD. In embodiments, the combination comprises naphazoline, 2C-B-FLY, and LP-211. In embodiments, the combination comprises naphazoline, 2C-B-FLY, and LP-44.

In embodiments, the combination comprises naphazoline, 5-MeO-AMT, and 5-CT. In embodiments, the combination comprises naphazoline, 5-MeO-AMT, and 5-MeO-DALT. In embodiments, the combination comprises naphazoline, 5-MeO-AMT, and 5-MeO-DMT. In embodiments, the combination comprises naphazoline, 5-MeO-AMT, and 5-MeO-MiPT. In embodiments, the combination comprises naphazoline, 5-MeO-AMT, and AGH-192. In embodiments, the combination comprises naphazoline, 5-MeO-AMT, and AGH-107. In embodiments, the combination comprises naphazoline, 5-MeO-AMT, and AMT. In embodiments, the combination comprises naphazoline, 5-MeO-AMT, and aripiprazole. In embodiments, the combination comprises naphazoline, 5-MeO-AMT, and AS-19. In embodiments, the combination comprises naphazoline, 5-MeO-AMT, and E-55888. In embodiments, the combination comprises naphazoline, 5-MeO-AMT, and LSD. In embodiments, the combination comprises naphazoline, 5-MeO-AMT, and LP-211. In embodiments, the combination comprises naphazoline, 5-MeO-AMT, and LP-44.

In embodiments, the combination comprises naphazoline, AL-37350A, and 5-CT. In embodiments, the combination comprises naphazoline, AL-37350A, and 5-MeO-DALT. In embodiments, the combination comprises naphazoline, AL-37350A, and 5-MeO-DMT In embodiments, the combination comprises naphazoline, AL-37350A, and 5-MeO-MiPT In embodiments, the combination comprises naphazoline, AL-37350A, and AGH-192. In embodiments, the combination comprises naphazoline, AL-37350A, and AGH-107. In embodiments, the combination comprises naphazoline, AL-37350A, and AMT. In embodiments, the combination comprises naphazoline, AL-37350A, and aripiprazole. In embodiments, the combination comprises naphazoline, AL-37350A, and AS-19. In embodiments, the combination comprises naphazoline, AL-37350A, and E-55888. In embodiments, the combination comprises naphazoline, AL-37350A, and LSD. In embodiments, the combination comprises naphazoline, AL-37350A, and LP-211. In embodiments, the combination comprises naphazoline, AL-37350A, and LP-44.

In embodiments, the combination comprises naphazoline, CP 809101, and 5-CT. In embodiments, the combination comprises naphazoline, CP 809101, and 5-MeO-DALT. In embodiments, the combination comprises naphazoline, CP 809101, and 5-MeO-DMT. In embodiments, the combination comprises naphazoline, CP 809101, and 5-MeO-MiPT In embodiments, the combination comprises naphazoline, CP 809101, and AGH-192. In embodiments, the combination comprises naphazoline, CP 809101, and AGH-107. In embodiments, the combination comprises naphazoline, CP 809101, and AMT. In embodiments, the combination comprises naphazoline, CP 809101, and aripiprazole. In embodiments, the combination comprises naphazoline, CP 809101, and AS-19. In embodiments, the combination comprises naphazoline, CP 809101, and E-55888. In embodiments, the combination comprises naphazoline, CP 809101, and LSD. In embodiments, the combination comprises naphazoline, CP 809101, and LP-211. In embodiments, the combination comprises naphazoline, CP 809101, and LP-44.

In embodiments, the combination comprises naphazoline, DALT, and 5-CT. In embodiments, the combination comprises naphazoline, DALT, and 5-MeO-DALT. In embodiments, the combination comprises naphazoline, DALT, and 5-MeO-DMT In embodiments, the combination comprises naphazoline, DALT, and 5-MeO-MiPT. In embodiments, the combination comprises naphazoline, DALT, and AGH-192. In embodiments, the combination comprises naphazoline, DALT, and AGH-107. In embodiments, the combination comprises naphazoline, DALT, and AMT. In embodiments, the combination comprises naphazoline, DALT, and aripiprazole. In embodiments, the combination comprises naphazoline, DALT, and AS-19. In embodiments, the combination comprises naphazoline, DALT, and E-55888. In embodiments, the combination comprises naphazoline, DALT, and LSD. In embodiments, the combination comprises naphazoline, DALT, and LP-211. In embodiments, the combination comprises naphazoline, DALT, and LP-44.

In embodiments, the combination comprises naphazoline, DOB, and 5-CT. In embodiments, the combination comprises naphazoline, DOB, and 5-MeO-DALT. In embodiments, the combination comprises naphazoline, DOB, and 5-MeO-DMT. In embodiments, the combination comprises naphazoline, DOB, and 5-MeO-MiPT. In embodiments, the combination comprises naphazoline, DOB, and AGH-192. In embodiments, the combination comprises naphazoline, DOB, and AGH-107. In embodiments, the combination comprises naphazoline, DOB, and AMT. In embodiments, the combination comprises naphazoline, DOB, and aripiprazole. In embodiments, the combination comprises naphazoline, DOB, and AS-19. In embodiments, the combination comprises naphazoline, DOB, and E-55888. In embodiments, the combination comprises naphazoline, DOB, and LSD. In embodiments, the combination comprises naphazoline, DOB, and LP-211. In embodiments, the combination comprises naphazoline, DOB, and LP-44.

In embodiments, the combination comprises naphazoline, DOET, and 5-CT. In embodiments, the combination comprises naphazoline, DOET, and 5-MeO-DALT. In embodiments, the combination comprises naphazoline, DOET, and 5-MeO-DMT In embodiments, the combination comprises naphazoline, DOET, and 5-MeO-MiPT. In embodiments, the combination comprises naphazoline, DOET, and AGH-192. In embodiments, the combination comprises naphazoline, DOET, and AGH-107. In embodiments, the combination comprises naphazoline, DOET, and AMT. In embodiments, the combination comprises naphazoline, DOET, and aripiprazole. In embodiments, the combination comprises naphazoline, DOET, and AS-19. In embodiments, the combination comprises naphazoline, DOET, and E-55888. In embodiments, the combination comprises naphazoline, DOET, and LSD. In embodiments, the combination comprises naphazoline, DOET, and LP-211. In embodiments, the combination comprises naphazoline, DOET, and LP-44.

In embodiments, the combination comprises naphazoline, DOHx, and 5-CT. In embodiments, the combination comprises naphazoline, DOHx, and 5-MeO-DALT. In embodiments, the combination comprises naphazoline, DOHx, and 5-MeO-DMT In embodiments, the combination comprises naphazoline, DOHx, and 5-MeO-MiPT. In embodiments, the combination comprises naphazoline, DOHx, and AGH-192. In embodiments, the combination comprises naphazoline, DOHx, and AGH-107. In embodiments, the combination comprises naphazoline, DOHx, and AMT. In embodiments, the combination comprises naphazoline, DOHx, and aripiprazole. In embodiments, the combination comprises naphazoline, DOHx, and AS-19. In embodiments, the combination comprises naphazoline, DOHx, and E-55888. In embodiments, the combination comprises naphazoline, DOHx, and LSD. In embodiments, the combination comprises naphazoline, DOHx, and LP-211. In embodiments, the combination comprises naphazoline, DOHx, and LP-44.

In embodiments, the combination comprises naphazoline, MEM, and 5-CT. In embodiments, the combination comprises naphazoline, MEM, and 5-MeO-DALT. In embodiments, the combination comprises naphazoline, MEM, and 5-MeO-DMT. In embodiments, the combination comprises naphazoline, MEM, and 5-MeO-MiPT. In embodiments, the combination comprises naphazoline, MEM, and AGH-192. In embodiments, the combination comprises naphazoline, MEM, and AGH-107. In embodiments, the combination comprises naphazoline, MEM, and AMT. In embodiments, the combination comprises naphazoline, MEM, and aripiprazole. In embodiments, the combination comprises naphazoline, MEM, and AS-19. In embodiments, the combination comprises naphazoline, MEM, and E-55888. In embodiments, the combination comprises naphazoline, MEM, and LSD. In embodiments, the combination comprises naphazoline, MEM, and LP-211. In embodiments, the combination comprises naphazoline, MEM, and LP-44.

In embodiments, the combination comprises naphazoline, CPP, and 5-CT. In embodiments, the combination comprises naphazoline, CPP, and 5-MeO-DALT. In embodiments, the combination comprises naphazoline, CPP, and 5-MeO-DMT In embodiments, the combination comprises naphazoline, CPP, and 5-MeO-MiPT. In embodiments, the combination comprises naphazoline, CPP, and AGH-192. In embodiments, the combination comprises naphazoline, CPP, and AGH-107. In embodiments, the combination comprises naphazoline, CPP, and AMT. In embodiments, the combination comprises naphazoline, CPP, and aripiprazole. In embodiments, the combination comprises naphazoline, CPP, and AS-19. In embodiments, the combination comprises naphazoline, CPP, and E-55888. In embodiments, the combination comprises naphazoline, CPP, and LSD. In embodiments, the combination comprises naphazoline, CPP, and LP-211. In embodiments, the combination comprises naphazoline, CPP, and LP-44.

In embodiments, the combination comprises naphazoline, NBOH-2C-CN, and 5-CT. In embodiments, the combination comprises naphazoline, NBOH-2C-CN, and 5-MeO-DALT. In embodiments, the combination comprises naphazoline, NBOH-2C-CN, and 5-MeO-DMT. In embodiments, the combination comprises naphazoline, NBOH-2C-CN, and 5-MeO-MiPT In embodiments, the combination comprises naphazoline, NBOH-2C-CN, and AGH-192. In embodiments, the combination comprises naphazoline, NBOH-2C-CN, and AGH-107. In embodiments, the combination comprises naphazoline, NBOH-2C-CN, and AMT. In embodiments, the combination comprises naphazoline, NBOH-2C-CN, and aripiprazole. In embodiments, the combination comprises naphazoline, NBOH-2C-CN, and AS-19. In embodiments, the combination comprises naphazoline, NBOH-2C-CN, and E-55888. In embodiments, the combination comprises naphazoline, NBOH-2C-CN, and LSD. In embodiments, the combination comprises naphazoline, NBOH-2C-CN, and LP-211. In embodiments, the combination comprises naphazoline, NBOH-2C-CN, and LP-44.

In embodiments, the combination comprises naphazoline, TCB-2, and 5-CT. In embodiments, the combination comprises naphazoline, TCB-2, and 5-MeO-DALT In embodiments, the combination comprises naphazoline, TCB-2, and 5-MeO-DMT. In embodiments, the combination comprises naphazoline, TCB-2, and 5-MeO-MiPT. In embodiments, the combination comprises naphazoline, TCB-2, and AGH-192. In embodiments, the combination comprises naphazoline, TCB-2, and AGH-107. In embodiments, the combination comprises naphazoline, TCB-2, and AMT. In embodiments, the combination comprises naphazoline, TCB-2, and aripiprazole. In embodiments, the combination comprises naphazoline, TCB-2, and AS-19. In embodiments, the combination comprises naphazoline, TCB-2, and E-55888. In embodiments, the combination comprises naphazoline, TCB-2, and LSD. In embodiments, the combination comprises naphazoline, TCB-2, and LP-211. In embodiments, the combination comprises naphazoline, TCB-2, and LP-44.

In embodiments, the combination comprises naphazoline, WAY 161503, and 5-CT. In embodiments, the combination comprises naphazoline, WAY 161503, and 5-MeO-DALT In embodiments, the combination comprises naphazoline, WAY 161503, and 5-MeO-DMT In embodiments, the combination comprises naphazoline, WAY 161503, and 5-MeO-MiPT. In embodiments, the combination comprises naphazoline, WAY 161503, and AGH-192. In embodiments, the combination comprises naphazoline, WAY 161503, and AGH-107. In embodiments, the combination comprises naphazoline, WAY 161503, and AMT. In embodiments, the combination comprises naphazoline, WAY 161503, and aripiprazole. In embodiments, the combination comprises naphazoline, WAY 161503, and AS-19. In embodiments, the combination comprises naphazoline, WAY 161503, and E-55888. In embodiments, the combination comprises naphazoline, WAY 161503, and LSD. In embodiments, the combination comprises naphazoline, WAY 161503, and LP-211. In embodiments, the combination comprises naphazoline, WAY 161503, and LP-44.

In embodiments, the combination comprises oxymetazoline, 1-methylpsilocin, and 5-CT. In embodiments, the combination comprises oxymetazoline, 1-methylpsilocin, and 5-MeO-DALT. In embodiments, the combination comprises oxymetazoline, 1-methylpsilocin, and 5-MeO-DMT. In embodiments, the combination comprises oxymetazoline, 1-methylpsilocin, and 5-MeO-MiPT In embodiments, the combination comprises oxymetazoline, 1-methylpsilocin, and AGH-192. In embodiments, the combination comprises oxymetazoline, 1-methylpsilocin, and AGH-107. In embodiments, the combination comprises oxymetazoline, 1-methylpsilocin, and AMT. In embodiments, the combination comprises oxymetazoline, 1-methylpsilocin, and aripiprazole. In embodiments, the combination comprises oxymetazoline, 1-methylpsilocin, and AS-19. In embodiments, the combination comprises oxymetazoline, 1-methylpsilocin, and E-55888. In embodiments, the combination comprises oxymetazoline, 1-methylpsilocin, and LSD. In embodiments, the combination comprises oxymetazoline, 1-methylpsilocin, and LP-211. In embodiments, the combination comprises oxymetazoline, 1-methylpsilocin, and LP-44.

In embodiments, the combination comprises oxymetazoline, 2C-B-FLY, and 5-CT. In embodiments, the combination comprises oxymetazoline, 2C-B-FLY, and 5-MeO-DALT. In embodiments, the combination comprises oxymetazoline, 2C-B-FLY, and 5-MeO-DMT. In embodiments, the combination comprises oxymetazoline, 2C-B-FLY, and 5-MeO-MiPT In embodiments, the combination comprises oxymetazoline, 2C-B-FLY, and AGH-192. In embodiments, the combination comprises oxymetazoline, 2C-B-FLY, and AGH-107. In embodiments, the combination comprises oxymetazoline, 2C-B-FLY, and AMT. In embodiments, the combination comprises oxymetazoline, 2C-B-FLY, and aripiprazole. In embodiments, the combination comprises oxymetazoline, 2C-B-FLY, and AS-19. In embodiments, the combination comprises oxymetazoline, 2C-B-FLY, and E-55888. In embodiments, the combination comprises oxymetazoline, 2C-B-FLY, and LSD. In embodiments, the combination comprises oxymetazoline, 2C-B-FLY, and LP-211. In embodiments, the combination comprises oxymetazoline, 2C-B-FLY, and LP-44.

In embodiments, the combination comprises oxymetazoline, 5-MeO-AMT, and 5-CT. In embodiments, the combination comprises oxymetazoline, 5-MeO-AMT, and 5-MeO-DALT. In embodiments, the combination comprises oxymetazoline, 5-MeO-AMT, and 5-MeO-DMT In embodiments, the combination comprises oxymetazoline, 5-MeO-AMT, and 5-MeO-MiPT. In embodiments, the combination comprises oxymetazoline, 5-MeO-AMT, and AGH-192. In embodiments, the combination comprises oxymetazoline, 5-MeO-AMT, and AGH-107. In embodiments, the combination comprises oxymetazoline, 5-MeO-AMT, and AMT. In embodiments, the combination comprises oxymetazoline, 5-MeO-AMT, and aripiprazole. In embodiments, the combination comprises oxymetazoline, 5-MeO-AMT, and AS-19. In embodiments, the combination comprises oxymetazoline, 5-MeO-AMT, and E-55888. In embodiments, the combination comprises oxymetazoline, 5-MeO-AMT, and LSD. In embodiments, the combination comprises oxymetazoline, 5-MeO-AMT, and LP-211. In embodiments, the combination comprises oxymetazoline, 5-MeO-AMT, and LP-44.

In embodiments, the combination comprises oxymetazoline, AL-37350A, and 5-CT. In embodiments, the combination comprises oxymetazoline, AL-37350A, and 5-MeO-DALT. In embodiments, the combination comprises oxymetazoline, AL-37350A, and 5-MeO-DMT. In embodiments, the combination comprises oxymetazoline, AL-37350A, and 5-MeO-MiPT In embodiments, the combination comprises oxymetazoline, AL-37350A, and AGH-192. In embodiments, the combination comprises oxymetazoline, AL-37350A, and AGH-107. In embodiments, the combination comprises oxymetazoline, AL-37350A, and AMT. In embodiments, the combination comprises oxymetazoline, AL-37350A, and aripiprazole. In embodiments, the combination comprises oxymetazoline, AL-37350A, and AS-19. In embodiments, the combination comprises oxymetazoline, AL-37350A, and E-55888. In embodiments, the combination comprises oxymetazoline, AL-37350A, and LSD. In embodiments, the combination comprises oxymetazoline, AL-37350A, and LP-211. In embodiments, the combination comprises oxymetazoline, AL-37350A, and LP-44.

In embodiments, the combination comprises oxymetazoline, CP 809101, and 5-CT. In embodiments, the combination comprises oxymetazoline, CP 809101, and 5-MeO-DALT. In embodiments, the combination comprises oxymetazoline, CP 809101, and 5-MeO-DMT. In embodiments, the combination comprises oxymetazoline, CP 809101, and 5-MeO-MiPT. In embodiments, the combination comprises oxymetazoline, CP 809101, and AGH-192. In embodiments, the combination comprises oxymetazoline, CP 809101, and AGH-107. In embodiments, the combination comprises oxymetazoline, CP 809101, and AMT. In embodiments, the combination comprises oxymetazoline, CP 809101, and aripiprazole. In embodiments, the combination comprises oxymetazoline, CP 809101, and AS-19. In embodiments, the combination comprises oxymetazoline, CP 809101, and E-55888. In embodiments, the combination comprises oxymetazoline, CP 809101, and LSD. In embodiments, the combination comprises oxymetazoline, CP 809101, and LP-211. In embodiments, the combination comprises oxymetazoline, CP 809101, and LP-44.

In embodiments, the combination comprises oxymetazoline, DALT, and 5-CT. In embodiments, the combination comprises oxymetazoline, DALT, and 5-MeO-DALT. In embodiments, the combination comprises oxymetazoline, DALT, and 5-MeO-DMT In embodiments, the combination comprises oxymetazoline, DALT, and 5-MeO-MiPT In embodiments, the combination comprises oxymetazoline, DALT, and AGH-192. In embodiments, the combination comprises oxymetazoline, DALT, and AGH-107. In embodiments, the combination comprises oxymetazoline, DALT, and AMT. In embodiments, the combination comprises oxymetazoline, DALT, and aripiprazole. In embodiments, the combination comprises oxymetazoline, DALT, and AS-19. In embodiments, the combination comprises oxymetazoline, DALT, and E-55888. In embodiments, the combination comprises oxymetazoline, DALT, and LSD. In embodiments, the combination comprises oxymetazoline, DALT, and LP-211. In embodiments, the combination comprises oxymetazoline, DALT, and LP-44.

In embodiments, the combination comprises oxymetazoline, DOB, and 5-CT. In embodiments, the combination comprises oxymetazoline, DOB, and 5-MeO-DALT. In embodiments, the combination comprises oxymetazoline, DOB, and 5-MeO-DMT In embodiments, the combination comprises oxymetazoline, DOB, and 5-MeO-MiPT. In embodiments, the combination comprises oxymetazoline, DOB, and AGH-192. In embodiments, the combination comprises oxymetazoline, DOB, and AGH-107. In embodiments, the combination comprises oxymetazoline, DOB, and AMT. In embodiments, the combination comprises oxymetazoline, DOB, and aripiprazole. In embodiments, the combination comprises oxymetazoline, DOB, and AS-19. In embodiments, the combination comprises oxymetazoline, DOB, and E-55888. In embodiments, the combination comprises oxymetazoline, DOB, and LSD. In embodiments, the combination comprises oxymetazoline, DOB, and LP-211. In embodiments, the combination comprises oxymetazoline, DOB, and LP-44.

In embodiments, the combination comprises oxymetazoline, DOET, and 5-CT. In embodiments, the combination comprises oxymetazoline, DOET, and 5-MeO-DALT. In embodiments, the combination comprises oxymetazoline, DOET, and 5-MeO-DMT In embodiments, the combination comprises oxymetazoline, DOET, and 5-MeO-MiPT. In embodiments, the combination comprises oxymetazoline, DOET, and AGH-192. In embodiments, the combination comprises oxymetazoline, DOET, and AGH-107. In embodiments, the combination comprises oxymetazoline, DOET, and AMT. In embodiments, the combination comprises oxymetazoline, DOET, and aripiprazole. In embodiments, the combination comprises oxymetazoline, DOET, and AS-19. In embodiments, the combination comprises oxymetazoline, DOET, and E-55888. In embodiments, the combination comprises oxymetazoline, DOET, and LSD. In embodiments, the combination comprises oxymetazoline, DOET, and LP-211. In embodiments, the combination comprises oxymetazoline, DOET, and LP-44.

In embodiments, the combination comprises oxymetazoline, DOHx, and 5-CT. In embodiments, the combination comprises oxymetazoline, DOHx, and 5-MeO-DALT. In embodiments, the combination comprises oxymetazoline, DOHx, and 5-MeO-DMT. In embodiments, the combination comprises oxymetazoline, DOHx, and 5-MeO-MiPT. In embodiments, the combination comprises oxymetazoline, DOHx, and AGH-192. In embodiments, the combination comprises oxymetazoline, DOHx, and AGH-107. In embodiments, the combination comprises oxymetazoline, DOHx, and AMT. In embodiments, the combination comprises oxymetazoline, DOHx, and aripiprazole. In embodiments, the combination comprises oxymetazoline, DOHx, and AS-19. In embodiments, the combination comprises oxymetazoline, DOHx, and E-55888. In embodiments, the combination comprises oxymetazoline, DOHx, and LSD. In embodiments, the combination comprises oxymetazoline, DOHx, and LP-211. In embodiments, the combination comprises oxymetazoline, DOHx, and LP-44.

In embodiments, the combination comprises oxymetazoline, MEM, and 5-CT. In embodiments, the combination comprises oxymetazoline, MEM, and 5-MeO-DALT. In embodiments, the combination comprises oxymetazoline, MEM, and 5-MeO-DMT. In embodiments, the combination comprises oxymetazoline, MEM, and 5-MeO-MiPT In embodiments, the combination comprises oxymetazoline, MEM, and AGH-192. In embodiments, the combination comprises oxymetazoline, MEM, and AGH-107. In embodiments, the combination comprises oxymetazoline, MEM, and AMT. In embodiments, the combination comprises oxymetazoline, MEM, and aripiprazole. In embodiments, the combination comprises oxymetazoline, MEM, and AS-19. In embodiments, the combination comprises oxymetazoline, MEM, and E-55888. In embodiments, the combination comprises oxymetazoline, MEM, and LSD. In embodiments, the combination comprises oxymetazoline, MEM, and LP-211. In embodiments, the combination comprises oxymetazoline, MEM, and LP-44.

In embodiments, the combination comprises oxymetazoline, CPP, and 5-CT. In embodiments, the combination comprises oxymetazoline, CPP, and 5-MeO-DALT. In embodiments, the combination comprises oxymetazoline, CPP, and 5-MeO-DMT. In embodiments, the combination comprises oxymetazoline, CPP, and 5-MeO-MiPT In embodiments, the combination comprises oxymetazoline, CPP, and AGH-192. In embodiments, the combination comprises oxymetazoline, CPP, and AGH-107. In embodiments, the combination comprises oxymetazoline, CPP, and AMT. In embodiments, the combination comprises oxymetazoline, CPP, and aripiprazole. In embodiments, the combination comprises oxymetazoline, CPP, and AS-19. In embodiments, the combination comprises oxymetazoline, CPP, and E-55888. In embodiments, the combination comprises oxymetazoline, CPP, and LSD. In embodiments, the combination comprises oxymetazoline, CPP, and LP-211. In embodiments, the combination comprises oxymetazoline, CPP, and LP-44.

In embodiments, the combination comprises oxymetazoline, NBOH-2C-CN, and 5-CT. In embodiments, the combination comprises oxymetazoline, NBOH-2C-CN, and 5-MeO-DALT. In embodiments, the combination comprises oxymetazoline, NBOH-2C-CN, and 5-MeO-DMT In embodiments, the combination comprises oxymetazoline, NBOH-2C-CN, and 5-MeO-MiPT. In embodiments, the combination comprises oxymetazoline, NBOH-2C-CN, and AGH-192. In embodiments, the combination comprises oxymetazoline, NBOH-2C-CN, and AGH-107. In embodiments, the combination comprises oxymetazoline, NBOH-2C-CN, and AMT. In embodiments, the combination comprises oxymetazoline, NBOH-2C-CN, and aripiprazole. In embodiments, the combination comprises oxymetazoline, NBOH-2C-CN, and AS-19. In embodiments, the combination comprises oxymetazoline, NBOH-2C-CN, and E-55888. In embodiments, the combination comprises oxymetazoline, NBOH-2C-CN, and LSD. In embodiments, the combination comprises oxymetazoline, NBOH-2C-CN, and LP-211. In embodiments, the combination comprises oxymetazoline, NBOH-2C-CN, and LP-44.

In embodiments, the combination comprises oxymetazoline, TCB-2, and 5-CT. In embodiments, the combination comprises oxymetazoline, TCB-2, and 5-MeO-DALT. In embodiments, the combination comprises oxymetazoline, TCB-2, and 5-MeO-DMT In embodiments, the combination comprises oxymetazoline, TCB-2, and 5-MeO-MiPT In embodiments, the combination comprises oxymetazoline, TCB-2, and AGH-192. In embodiments, the combination comprises oxymetazoline, TCB-2, and AGH-107. In embodiments, the combination comprises oxymetazoline, TCB-2, and AMT. In embodiments, the combination comprises oxymetazoline, TCB-2, and aripiprazole. In embodiments, the combination comprises oxymetazoline, TCB-2, and AS-19. In embodiments, the combination comprises oxymetazoline, TCB-2, and E-55888. In embodiments, the combination comprises oxymetazoline, TCB-2, and LSD. In embodiments, the combination comprises oxymetazoline, TCB-2, and LP-211. In embodiments, the combination comprises oxymetazoline, TCB-2, and LP-44.

In embodiments, the combination comprises oxymetazoline, WAY 161503, and 5-CT. In embodiments, the combination comprises oxymetazoline, WAY 161503, and 5-MeO-DALT. In embodiments, the combination comprises oxymetazoline, WAY 161503, and 5-MeO-DMT. In embodiments, the combination comprises oxymetazoline, WAY 161503, and 5-MeO-MiPT In embodiments, the combination comprises oxymetazoline, WAY 161503, and AGH-192. In embodiments, the combination comprises oxymetazoline, WAY 161503, and AGH-107. In embodiments, the combination comprises oxymetazoline, WAY 161503, and AMT. In embodiments, the combination comprises oxymetazoline, WAY 161503, and aripiprazole. In embodiments, the combination comprises oxymetazoline, WAY 161503, and AS-19. In embodiments, the combination comprises oxymetazoline, WAY 161503, and E-55888. In embodiments, the combination comprises oxymetazoline, WAY 161503, and LSD. In embodiments, the combination comprises oxymetazoline, WAY 161503, and LP-211. In embodiments, the combination comprises oxymetazoline, WAY 161503, and LP-44.

In embodiments, the combination comprises rilmenidine, 1-methylpsilocin, and 5-CT. In embodiments, the combination comprises rilmenidine, 1-methylpsilocin, and 5-MeO-DALT. In embodiments, the combination comprises rilmenidine, 1-methylpsilocin, and 5-MeO-DMT In embodiments, the combination comprises rilmenidine, 1-methylpsilocin, and 5-MeO-MiPT. In embodiments, the combination comprises rilmenidine, 1-methylpsilocin, and AGH-192. In embodiments, the combination comprises rilmenidine, 1-methylpsilocin, and AGH-107. In embodiments, the combination comprises rilmenidine, 1-methylpsilocin, and AMT. In embodiments, the combination comprises rilmenidine, 1-methylpsilocin, and aripiprazole. In embodiments, the combination comprises rilmenidine, 1-methylpsilocin, and AS-19. In embodiments, the combination comprises rilmenidine, 1-methylpsilocin, and E-55888. In embodiments, the combination comprises rilmenidine, 1-methylpsilocin, and LSD. In embodiments, the combination comprises rilmenidine, 1-methylpsilocin, and LP-211. In embodiments, the combination comprises rilmenidine, 1-methylpsilocin, and LP-44.

In embodiments, the combination comprises rilmenidine, 2C-B-FLY, and 5-CT. In embodiments, the combination comprises rilmenidine, 2C-B-FLY, and 5-MeO-DALT. In embodiments, the combination comprises rilmenidine, 2C-B-FLY, and 5-MeO-DMT In embodiments, the combination comprises rilmenidine, 2C-B-FLY, and 5-MeO-MiPT In embodiments, the combination comprises rilmenidine, 2C-B-FLY, and AGH-192. In embodiments, the combination comprises rilmenidine, 2C-B-FLY, and AGH-107. In embodiments, the combination comprises rilmenidine, 2C-B-FLY, and AMT. In embodiments, the combination comprises rilmenidine, 2C-B-FLY, and aripiprazole. In embodiments, the combination comprises rilmenidine, 2C-B-FLY, and AS-19. In embodiments, the combination comprises rilmenidine, 2C-B-FLY, and E-55888. In embodiments, the combination comprises rilmenidine, 2C-B-FLY, and LSD. In embodiments, the combination comprises rilmenidine, 2C-B-FLY, and LP-211. In embodiments, the combination comprises rilmenidine, 2C-B-FLY, and LP-44.

In embodiments, the combination comprises rilmenidine, 5-MeO-AMT, and 5-CT. In embodiments, the combination comprises rilmenidine, 5-MeO-AMT, and 5-MeO-DALT. In embodiments, the combination comprises rilmenidine, 5-MeO-AMT, and 5-MeO-DMT In embodiments, the combination comprises rilmenidine, 5-MeO-AMT, and 5-MeO-MiPT. In embodiments, the combination comprises rilmenidine, 5-MeO-AMT, and AGH-192. In embodiments, the combination comprises rilmenidine, 5-MeO-AMT, and AGH-107. In embodiments, the combination comprises rilmenidine, 5-MeO-AMT, and AMT. In embodiments, the combination comprises rilmenidine, 5-MeO-AMT, and aripiprazole. In embodiments, the combination comprises rilmenidine, 5-MeO-AMT, and AS-19. In embodiments, the combination comprises rilmenidine, 5-MeO-AMT, and E-55888. In embodiments, the combination comprises rilmenidine, 5-MeO-AMT, and LSD. In embodiments, the combination comprises rilmenidine, 5-MeO-AMT, and LP-211. In embodiments, the combination comprises rilmenidine, 5-MeO-AMT, and LP-44.

In embodiments, the combination comprises rilmenidine, AL-37350A, and 5-CT. In embodiments, the combination comprises rilmenidine, AL-37350A, and 5-MeO-DALT. In embodiments, the combination comprises rilmenidine, AL-37350A, and 5-MeO-DMT. In embodiments, the combination comprises rilmenidine, AL-37350A, and 5-MeO-MiPT In embodiments, the combination comprises rilmenidine, AL-37350A, and AGH-192. In embodiments, the combination comprises rilmenidine, AL-37350A, and AGH-107. In embodiments, the combination comprises rilmenidine, AL-37350A, and AMT. In embodiments, the combination comprises rilmenidine, AL-37350A, and aripiprazole. In embodiments, the combination comprises rilmenidine, AL-37350A, and AS-19. In embodiments, the combination comprises rilmenidine, AL-37350A, and E-55888. In embodiments, the combination comprises rilmenidine, AL-37350A, and LSD. In embodiments, the combination comprises rilmenidine, AL-37350A, and LP-211. In embodiments, the combination comprises rilmenidine, AL-37350A, and LP-44.

In embodiments, the combination comprises rilmenidine, CP 809101, and 5-CT. In embodiments, the combination comprises rilmenidine, CP 809101, and 5-MeO-DALT. In embodiments, the combination comprises rilmenidine, CP 809101, and 5-MeO-DMT In embodiments, the combination comprises rilmenidine, CP 809101, and 5-MeO-MiPT In embodiments, the combination comprises rilmenidine, CP 809101, and AGH-192. In embodiments, the combination comprises rilmenidine, CP 809101, and AGH-107. In embodiments, the combination comprises rilmenidine, CP 809101, and AMT. In embodiments, the combination comprises rilmenidine, CP 809101, and aripiprazole. In embodiments, the combination comprises rilmenidine, CP 809101, and AS-19. In embodiments, the combination comprises rilmenidine, CP 809101, and E-55888. In embodiments, the combination comprises rilmenidine, CP 809101, and LSD. In embodiments, the combination comprises rilmenidine, CP 809101, and LP-211. In embodiments, the combination comprises rilmenidine, CP 809101, and LP-44.

In embodiments, the combination comprises rilmenidine, DALT, and 5-CT. In embodiments, the combination comprises rilmenidine, DALT, and 5-MeO-DALT. In embodiments, the combination comprises rilmenidine, DALT, and 5-MeO-DMT. In embodiments, the combination comprises rilmenidine, DALT, and 5-MeO-MiPT. In embodiments, the combination comprises rilmenidine, DALT, and AGH-192. In embodiments, the combination comprises rilmenidine, DALT, and AGH-107. In embodiments, the combination comprises rilmenidine, DALT, and AMT. In embodiments, the combination comprises rilmenidine, DALT, and aripiprazole. In embodiments, the combination comprises rilmenidine, DALT, and AS-19. In embodiments, the combination comprises rilmenidine, DALT, and E-55888. In embodiments, the combination comprises rilmenidine, DALT, and LSD. In embodiments, the combination comprises rilmenidine, DALT, and LP-211. In embodiments, the combination comprises rilmenidine, DALT, and LP-44.

In embodiments, the combination comprises rilmenidine, DOB, and 5-CT. In embodiments, the combination comprises rilmenidine, DOB, and 5-MeO-DALT. In embodiments, the combination comprises rilmenidine, DOB, and 5-MeO-DMT. In embodiments, the combination comprises rilmenidine, DOB, and 5-MeO-MiPT. In embodiments, the combination comprises rilmenidine, DOB, and AGH-192. In embodiments, the combination comprises rilmenidine, DOB, and AGH-107. In embodiments, the combination comprises rilmenidine, DOB, and AMT. In embodiments, the combination comprises rilmenidine, DOB, and aripiprazole. In embodiments, the combination comprises rilmenidine, DOB, and AS-19. In embodiments, the combination comprises rilmenidine, DOB, and E-55888. In embodiments, the combination comprises rilmenidine, DOB, and LSD. In embodiments, the combination comprises rilmenidine, DOB, and LP-211. In embodiments, the combination comprises rilmenidine, DOB, and LP-44.

In embodiments, the combination comprises rilmenidine, DOET, and 5-CT. In embodiments, the combination comprises rilmenidine, DOET, and 5-MeO-DALT. In embodiments, the combination comprises rilmenidine, DOET, and 5-MeO-DMT. In embodiments, the combination comprises rilmenidine, DOET, and 5-MeO-MiPT. In embodiments, the combination comprises rilmenidine, DOET, and AGH-192. In embodiments, the combination comprises rilmenidine, DOET, and AGH-107. In embodiments, the combination comprises rilmenidine, DOET, and AMT. In embodiments, the combination comprises rilmenidine, DOET, and aripiprazole. In embodiments, the combination comprises rilmenidine, DOET, and AS-19. In embodiments, the combination comprises rilmenidine, DOET, and E-55888. In embodiments, the combination comprises rilmenidine, DOET, and LSD. In embodiments, the combination comprises rilmenidine, DOET, and LP-211. In embodiments, the combination comprises rilmenidine, DOET, and LP-44.

In embodiments, the combination comprises rilmenidine, DOHx, and 5-CT. In embodiments, the combination comprises rilmenidine, DOHx, and 5-MeO-DALT. In embodiments, the combination comprises rilmenidine, DOHx, and 5-MeO-DMT. In embodiments, the combination comprises rilmenidine, DOHx, and 5-MeO-MiPT. In embodiments, the combination comprises rilmenidine, DOHx, and AGH-192. In embodiments, the combination comprises rilmenidine, DOHx, and AGH-107. In embodiments, the combination comprises rilmenidine, DOHx, and AMT. In embodiments, the combination comprises rilmenidine, DOHx, and aripiprazole. In embodiments, the combination comprises rilmenidine, DOHx, and AS-19. In embodiments, the combination comprises rilmenidine, DOHx, and E-55888. In embodiments, the combination comprises rilmenidine, DOHx, and LSD. In embodiments, the combination comprises rilmenidine, DOHx, and LP-211. In embodiments, the combination comprises rilmenidine, DOHx, and LP-44.

In embodiments, the combination comprises rilmenidine, MEM, and 5-CT. In embodiments, the combination comprises rilmenidine, MEM, and 5-MeO-DALT. In embodiments, the combination comprises rilmenidine, MEM, and 5-MeO-DMT In embodiments, the combination comprises rilmenidine, MEM, and 5-MeO-MiPT. In embodiments, the combination comprises rilmenidine, MEM, and AGH-192. In embodiments, the combination comprises rilmenidine, MEM, and AGH-107. In embodiments, the combination comprises rilmenidine, MEM, and AMT. In embodiments, the combination comprises rilmenidine, MEM, and aripiprazole. In embodiments, the combination comprises rilmenidine, MEM, and AS-19. In embodiments, the combination comprises rilmenidine, MEM, and E-55888. In embodiments, the combination comprises rilmenidine, MEM, and LSD. In embodiments, the combination comprises rilmenidine, MEM, and LP-211. In embodiments, the combination comprises rilmenidine, MEM, and LP-44.

In embodiments, the combination comprises rilmenidine, CPP, and 5-CT. In embodiments, the combination comprises rilmenidine, CPP, and 5-MeO-DALT. In embodiments, the combination comprises rilmenidine, CPP, and 5-MeO-DMT. In embodiments, the combination comprises rilmenidine, CPP, and 5-MeO-MiPT. In embodiments, the combination comprises rilmenidine, CPP, and AGH-192. In embodiments, the combination comprises rilmenidine, CPP, and AGH-107. In embodiments, the combination comprises rilmenidine, CPP, and AMT. In embodiments, the combination comprises rilmenidine, CPP, and aripiprazole. In embodiments, the combination comprises rilmenidine, CPP, and AS-19. In embodiments, the combination comprises rilmenidine, CPP, and E-55888. In embodiments, the combination comprises rilmenidine, CPP, and LSD. In embodiments, the combination comprises rilmenidine, CPP, and LP-211. In embodiments, the combination comprises rilmenidine, CPP, and LP-44.

In embodiments, the combination comprises rilmenidine, NBOH-2C-CN, and 5-CT. In embodiments, the combination comprises rilmenidine, NBOH-2C-CN, and 5-MeO-DALT. In embodiments, the combination comprises rilmenidine, NBOH-2C-CN, and 5-MeO-DMT In embodiments, the combination comprises rilmenidine, NBOH-2C-CN, and 5-MeO-MiPT In embodiments, the combination comprises rilmenidine, NBOH-2C-CN, and AGH-192. In embodiments, the combination comprises rilmenidine, NBOH-2C-CN, and AGH-107. In embodiments, the combination comprises rilmenidine, NBOH-2C-CN, and AMT. In embodiments, the combination comprises rilmenidine, NBOH-2C-CN, and aripiprazole. In embodiments, the combination comprises rilmenidine, NBOH-2C-CN, and AS-19. In embodiments, the combination comprises rilmenidine, NBOH-2C-CN, and E-55888. In embodiments, the combination comprises rilmenidine, NBOH-2C-CN, and LSD. In embodiments, the combination comprises rilmenidine, NBOH-2C-CN, and LP-211. In embodiments, the combination comprises rilmenidine, NBOH-2C-CN, and LP-44.

In embodiments, the combination comprises rilmenidine, TCB-2, and 5-CT. In embodiments, the combination comprises rilmenidine, TCB-2, and 5-MeO-DALT. In embodiments, the combination comprises rilmenidine, TCB-2, and 5-MeO-DMT In embodiments, the combination comprises rilmenidine, TCB-2, and 5-MeO-MiPT. In embodiments, the combination comprises rilmenidine, TCB-2, and AGH-192. In embodiments, the combination comprises rilmenidine, TCB-2, and AGH-107. In embodiments, the combination comprises rilmenidine, TCB-2, and AMT. In embodiments, the combination comprises rilmenidine, TCB-2, and aripiprazole. In embodiments, the combination comprises rilmenidine, TCB-2, and AS-19. In embodiments, the combination comprises rilmenidine, TCB-2, and E-55888. In embodiments, the combination comprises rilmenidine, TCB-2, and LSD. In embodiments, the combination comprises rilmenidine, TCB-2, and LP-211. In embodiments, the combination comprises rilmenidine, TCB-2, and LP-44.

In embodiments, the combination comprises rilmenidine, WAY 161503, and 5-CT. In embodiments, the combination comprises rilmenidine, WAY 161503, and 5-MeO-DALT. In embodiments, the combination comprises rilmenidine, WAY 161503, and 5-MeO-DMT In embodiments, the combination comprises rilmenidine, WAY 161503, and 5-MeO-MiPT In embodiments, the combination comprises rilmenidine, WAY 161503, and AGH-192. In embodiments, the combination comprises rilmenidine, WAY 161503, and AGH-107. In embodiments, the combination comprises rilmenidine, WAY 161503, and AMT. In embodiments, the combination comprises rilmenidine, WAY 161503, and aripiprazole. In embodiments, the combination comprises rilmenidine, WAY 161503, and AS-19. In embodiments, the combination comprises rilmenidine, WAY 161503, and E-55888. In embodiments, the combination comprises rilmenidine, WAY 161503, and LSD. In embodiments, the combination comprises rilmenidine, WAY 161503, and LP-211. In embodiments, the combination comprises rilmenidine, WAY 161503, and LP-44.

In embodiments, the combination comprises tetryzoline, 1-methylpsilocin, and 5-CT. In embodiments, the combination comprises tetryzoline, 1-methylpsilocin, and 5-MeO-DALT. In embodiments, the combination comprises tetryzoline, 1-methylpsilocin, and 5-MeO-DMT. In embodiments, the combination comprises tetryzoline, 1-methylpsilocin, and 5-MeO-MiPT. In embodiments, the combination comprises tetryzoline, 1-methylpsilocin, and AGH-192. In embodiments, the combination comprises tetryzoline, 1-methylpsilocin, and AGH-107. In embodiments, the combination comprises tetryzoline, 1-methylpsilocin, and AMT. In embodiments, the combination comprises tetryzoline, 1-methylpsilocin, and aripiprazole. In embodiments, the combination comprises tetryzoline, 1-methylpsilocin, and AS-19. In embodiments, the combination comprises tetryzoline, 1-methylpsilocin, and E-55888. In embodiments, the combination comprises tetryzoline, 1-methylpsilocin, and LSD. In embodiments, the combination comprises tetryzoline, 1-methyl psilocin, and LP-211. In embodiments, the combination comprises tetryzoline, 1-methylpsilocin, and LP-44.

In embodiments, the combination comprises tetryzoline, 2C-B-FLY, and 5-CT. In embodiments, the combination comprises tetryzoline, 2C-B-FLY, and 5-MeO-DALT. In embodiments, the combination comprises tetryzoline, 2C-B-FLY, and 5-MeO-DMT In embodiments, the combination comprises tetryzoline, 2C-B-FLY, and 5-MeO-MiPT. In embodiments, the combination comprises tetryzoline, 2C-B-FLY, and AGH-192. In embodiments, the combination comprises tetryzoline, 2C-B-FLY, and AGH-107. In embodiments, the combination comprises tetryzoline, 2C-B-FLY, and AMT. In embodiments, the combination comprises tetryzoline, 2C-B-FLY, and aripiprazole. In embodiments, the combination comprises tetryzoline, 2C-B-FLY, and AS-19. In embodiments, the combination comprises tetryzoline, 2C-B-FLY, and E-55888. In embodiments, the combination comprises tetryzoline, 2C-B-FLY, and LSD. In embodiments, the combination comprises tetryzoline, 2C-B-FLY, and LP-211. In embodiments, the combination comprises tetryzoline, 2C-B-FLY, and LP-44.

In embodiments, the combination comprises tetryzoline, 5-MeO-AMT, and 5-CT. In embodiments, the combination comprises tetryzoline, 5-MeO-AMT, and 5-MeO-DALT. In embodiments, the combination comprises tetryzoline, 5-MeO-AMT, and 5-MeO-DMT In embodiments, the combination comprises tetryzoline, 5-MeO-AMT, and 5-MeO-MiPT. In embodiments, the combination comprises tetryzoline, 5-MeO-AMT, and AGH-192. In embodiments, the combination comprises tetryzoline, 5-MeO-AMT, and AGH-107. In embodiments, the combination comprises tetryzoline, 5-MeO-AMT, and AMT. In embodiments, the combination comprises tetryzoline, 5-MeO-AMT, and aripiprazole. In embodiments, the combination comprises tetryzoline, 5-MeO-AMT, and AS-19. In embodiments, the combination comprises tetryzoline, 5-MeO-AMT, and E-55888. In embodiments, the combination comprises tetryzoline, 5-MeO-AMT, and LSD. In embodiments, the combination comprises tetryzoline, 5-MeO-AMT, and LP-211. In embodiments, the combination comprises tetryzoline, 5-MeO-AMT, and LP-44.

In embodiments, the combination comprises tetryzoline, AL-37350A, and 5-CT. In embodiments, the combination comprises tetryzoline, AL-37350A, and 5-MeO-DALT. In embodiments, the combination comprises tetryzoline, AL-37350A, and 5-MeO-DMT. In embodiments, the combination comprises tetryzoline, AL-37350A, and 5-MeO-MiPT. In embodiments, the combination comprises tetryzoline, AL-37350A, and AGH-192. In embodiments, the combination comprises tetryzoline, AL-37350A, and AGH-107. In embodiments, the combination comprises tetryzoline, AL-37350A, and AMT. In embodiments, the combination comprises tetryzoline, AL-37350A, and aripiprazole. In embodiments, the combination comprises tetryzoline, AL-37350A, and AS-19. In embodiments, the combination comprises tetryzoline, AL-37350A, and E-55888. In embodiments, the combination comprises tetryzoline, AL-37350A, and LSD. In embodiments, the combination comprises tetryzoline, AL-37350A, and LP-211. In embodiments, the combination comprises tetryzoline, AL-37350A, and LP-44.

In embodiments, the combination comprises tetryzoline, CP 809101, and 5-CT. In embodiments, the combination comprises tetryzoline, CP 809101, and 5-MeO-DALT. In embodiments, the combination comprises tetryzoline, CP 809101, and 5-MeO-DMT. In embodiments, the combination comprises tetryzoline, CP 809101, and 5-MeO-MiPT In embodiments, the combination comprises tetryzoline, CP 809101, and AGH-192. In embodiments, the combination comprises tetryzoline, CP 809101, and AGH-107. In embodiments, the combination comprises tetryzoline, CP 809101, and AMT. In embodiments, the combination comprises tetryzoline, CP 809101, and aripiprazole. In embodiments, the combination comprises tetryzoline, CP 809101, and AS-19. In embodiments, the combination comprises tetryzoline, CP 809101, and E-55888. In embodiments, the combination comprises tetryzoline, CP 809101, and LSD. In embodiments, the combination comprises tetryzoline, CP 809101, and LP-211. In embodiments, the combination comprises tetryzoline, CP 809101, and LP-44.

In embodiments, the combination comprises tetryzoline, DALT, and 5-CT. In embodiments, the combination comprises tetryzoline, DALT, and 5-MeO-DALT. In embodiments, the combination comprises tetryzoline, DALT, and 5-MeO-DMT. In embodiments, the combination comprises tetryzoline, DALT, and 5-MeO-MiPT. In embodiments, the combination comprises tetryzoline, DALT, and AGH-192. In embodiments, the combination comprises tetryzoline, DALT, and AGH-107. In embodiments, the combination comprises tetryzoline, DALT, and AMT. In embodiments, the combination comprises tetryzoline, DALT, and aripiprazole. In embodiments, the combination comprises tetryzoline, DALT, and AS-19. In embodiments, the combination comprises tetryzoline, DALT, and E-55888. In embodiments, the combination comprises tetryzoline, DALT, and LSD. In embodiments, the combination comprises tetryzoline, DALT, and LP-211. In embodiments, the combination comprises tetryzoline, DALT, and LP-44.

In embodiments, the combination comprises tetryzoline, DOB, and 5-CT. In embodiments, the combination comprises tetryzoline, DOB, and 5-MeO-DALT. In embodiments, the combination comprises tetryzoline, DOB, and 5-MeO-DMT In embodiments, the combination comprises tetryzoline, DOB, and 5-MeO-MiPT. In embodiments, the combination comprises tetryzoline, DOB, and AGH-192. In embodiments, the combination comprises tetryzoline, DOB, and AGH-107. In embodiments, the combination comprises tetryzoline, DOB, and AMT. In embodiments, the combination comprises tetryzoline, DOB, and aripiprazole. In embodiments, the combination comprises tetryzoline, DOB, and AS-19. In embodiments, the combination comprises tetryzoline, DOB, and E-55888. In embodiments, the combination comprises tetryzoline, DOB, and LSD. In embodiments, the combination comprises tetryzoline, DOB, and LP-211. In embodiments, the combination comprises tetryzoline, DOB, and LP-44.

In embodiments, the combination comprises tetryzoline, DOET, and 5-CT. In embodiments, the combination comprises tetryzoline, DOET, and 5-MeO-DALT. In embodiments, the combination comprises tetryzoline, DOET, and 5-MeO-DMT In embodiments, the combination comprises tetryzoline, DOET, and 5-MeO-MiPT. In embodiments, the combination comprises tetryzoline, DOET, and AGH-192. In embodiments, the combination comprises tetryzoline, DOET, and AGH-107. In embodiments, the combination comprises tetryzoline, DOET, and AMT. In embodiments, the combination comprises tetryzoline, DOET, and aripiprazole. In embodiments, the combination comprises tetryzoline, DOET, and AS-19. In embodiments, the combination comprises tetryzoline, DOET, and E-55888. In embodiments, the combination comprises tetryzoline, DOET, and LSD. In embodiments, the combination comprises tetryzoline, DOET, and LP-211. In embodiments, the combination comprises tetryzoline, DOET, and LP-44.

In embodiments, the combination comprises tetryzoline, DOHx, and 5-CT. In embodiments, the combination comprises tetryzoline, DOHx, and 5-MeO-DALT. In embodiments, the combination comprises tetryzoline, DOHx, and 5-MeO-DMT. In embodiments, the combination comprises tetryzoline, DOHx, and 5-MeO-MiPT. In embodiments, the combination comprises tetryzoline, DOHx, and AGH-192. In embodiments, the combination comprises tetryzoline, DOHx, and AGH-107. In embodiments, the combination comprises tetryzoline, DOHx, and AMT. In embodiments, the combination comprises tetryzoline, DOHx, and aripiprazole. In embodiments, the combination comprises tetryzoline, DOHx, and AS-19. In embodiments, the combination comprises tetryzoline, DOHx, and E-55888. In embodiments, the combination comprises tetryzoline, DOHx, and LSD. In embodiments, the combination comprises tetryzoline, DOHx, and LP-211. In embodiments, the combination comprises tetryzoline, DOHx, and LP-44.

In embodiments, the combination comprises tetryzoline, MEM, and 5-CT. In embodiments, the combination comprises tetryzoline, MEM, and 5-MeO-DALT. In embodiments, the combination comprises tetryzoline, MEM, and 5-MeO-DMT In embodiments, the combination comprises tetryzoline, MEM, and 5-MeO-MiPT. In embodiments, the combination comprises tetryzoline, MEM, and AGH-192. In embodiments, the combination comprises tetryzoline, MEM, and AGH-107. In embodiments, the combination comprises tetryzoline, MEM, and AMT. In embodiments, the combination comprises tetryzoline, MEM, and aripiprazole. In embodiments, the combination comprises tetryzoline, MEM, and AS-19. In embodiments, the combination comprises tetryzoline, MEM, and E-55888. In embodiments, the combination comprises tetryzoline, MEM, and LSD. In embodiments, the combination comprises tetryzoline, MEM, and LP-211. In embodiments, the combination comprises tetryzoline, MEM, and LP-44.

In embodiments, the combination comprises tetryzoline, CPP, and 5-CT. In embodiments, the combination comprises tetryzoline, CPP, and 5-MeO-DALT. In embodiments, the combination comprises tetryzoline, CPP, and 5-MeO-DMT. In embodiments, the combination comprises tetryzoline, CPP, and 5-MeO-MiPT. In embodiments, the combination comprises tetryzoline, CPP, and AGH-192. In embodiments, the combination comprises tetryzoline, CPP, and AGH-107. In embodiments, the combination comprises tetryzoline, CPP, and AMT. In embodiments, the combination comprises tetryzoline, CPP, and aripiprazole. In embodiments, the combination comprises tetryzoline, CPP, and AS-19. In embodiments, the combination comprises tetryzoline, CPP, and E-55888. In embodiments, the combination comprises tetryzoline, CPP, and LSD. In embodiments, the combination comprises tetryzoline, CPP, and LP-211. In embodiments, the combination comprises tetryzoline, CPP, and LP-44.

In embodiments, the combination comprises tetryzoline, NBOH-2C-CN, and 5-CT. In embodiments, the combination comprises tetryzoline, NBOH-2C-CN, and 5-MeO-DALT. In embodiments, the combination comprises tetryzoline, NBOH-2C-CN, and 5-MeO-DMT. In embodiments, the combination comprises tetryzoline, NBOH-2C-CN, and 5-MeO-MiPT. In embodiments, the combination comprises tetryzoline, NBOH-2C-CN, and AGH-192. In embodiments, the combination comprises tetryzoline, NBOH-2C-CN, and AGH-107. In embodiments, the combination comprises tetryzoline, NBOH-2C-CN, and AMT. In embodiments, the combination comprises tetryzoline, NBOH-2C-CN, and aripiprazole. In embodiments, the combination comprises tetryzoline, NBOH-2C-CN, and AS-19. In embodiments, the combination comprises tetryzoline, NBOH-2C-CN, and E-55888. In embodiments, the combination comprises tetryzoline, NBOH-2C-CN, and LSD. In embodiments, the combination comprises tetryzoline, NBOH-2C-CN, and LP-211. In embodiments, the combination comprises tetryzoline, NBOH-2C-CN, and LP-44.

In embodiments, the combination comprises tetryzoline, TCB-2, and 5-CT. In embodiments, the combination comprises tetryzoline, TCB-2, and 5-MeO-DALT. In embodiments, the combination comprises tetryzoline, TCB-2, and 5-MeO-DMT. In embodiments, the combination comprises tetryzoline, TCB-2, and 5-MeO-MiPT. In embodiments, the combination comprises tetryzoline, TCB-2, and AGH-192. In embodiments, the combination comprises tetryzoline, TCB-2, and AGH-107. In embodiments, the combination comprises tetryzoline, TCB-2, and AMT. In embodiments, the combination comprises tetryzoline, TCB-2, and aripiprazole. In embodiments, the combination comprises tetryzoline, TCB-2, and AS-19. In embodiments, the combination comprises tetryzoline, TCB-2, and E-55888. In embodiments, the combination comprises tetryzoline, TCB-2, and LSD. In embodiments, the combination comprises tetryzoline, TCB-2, and LP-211. In embodiments, the combination comprises tetryzoline, TCB-2, and LP-44.

In embodiments, the combination comprises tetryzoline, WAY 161503, and 5-CT. In embodiments, the combination comprises tetryzoline, WAY 161503, and 5-MeO-DALT. In embodiments, the combination comprises tetryzoline, WAY 161503, and 5-MeO-DMT In embodiments, the combination comprises tetryzoline, WAY 161503, and 5-MeO-MiPT In embodiments, the combination comprises tetryzoline, WAY 161503, and AGH-192. In embodiments, the combination comprises tetryzoline, WAY 161503, and AGH-107. In embodiments, the combination comprises tetryzoline, WAY 161503, and AMT. In embodiments, the combination comprises tetryzoline, WAY 161503, and aripiprazole. In embodiments, the combination comprises tetryzoline, WAY 161503, and AS-19. In embodiments, the combination comprises tetryzoline, WAY 161503, and E-55888. In embodiments, the combination comprises tetryzoline, WAY 161503, and LSD. In embodiments, the combination comprises tetryzoline, WAY 161503, and LP-211. In embodiments, the combination comprises tetryzoline, WAY 161503, and LP-44.

In embodiments, the combination comprises tolonidine, 1-methylpsilocin, and 5-CT. In embodiments, the combination comprises tolonidine, 1-methylpsilocin, and 5-MeO-DALT. In embodiments, the combination comprises tolonidine, 1-methylpsilocin, and 5-MeO-DMT In embodiments, the combination comprises tolonidine, 1-methylpsilocin, and 5-MeO-MiPT In embodiments, the combination comprises tolonidine, 1-methylpsilocin, and AGH-192. In embodiments, the combination comprises tolonidine, 1-methylpsilocin, and AGH-107. In embodiments, the combination comprises tolonidine, 1-methylpsilocin, and AMT. In embodiments, the combination comprises tolonidine, 1-methylpsilocin, and aripiprazole. In embodiments, the combination comprises tolonidine, 1-methylpsilocin, and AS-19. In embodiments, the combination comprises tolonidine, 1-methylpsilocin, and E-55888. In embodiments, the combination comprises tolonidine, 1-methylpsilocin, and LSD. In embodiments, the combination comprises tolonidine, 1-methyl psilocin, and LP-211. In embodiments, the combination comprises tolonidine, 1-methylpsilocin, and LP-44.

In embodiments, the combination comprises tolonidine, 2C-B-FLY, and 5-CT. In embodiments, the combination comprises tolonidine, 2C-B-FLY, and 5-MeO-DALT. In embodiments, the combination comprises tolonidine, 2C-B-FLY, and 5-MeO-DMT In embodiments, the combination comprises tolonidine, 2C-B-FLY, and 5-MeO-MiPT. In embodiments, the combination comprises tolonidine, 2C-B-FLY, and AGH-192. In embodiments, the combination comprises tolonidine, 2C-B-FLY, and AGH-107. In embodiments, the combination comprises tolonidine, 2C-B-FLY, and AMT. In embodiments, the combination comprises tolonidine, 2C-B-FLY, and aripiprazole. In embodiments, the combination comprises tolonidine, 2C-B-FLY, and AS-19. In embodiments, the combination comprises tolonidine, 2C-B-FLY, and E-55888. In embodiments, the combination comprises tolonidine, 2C-B-FLY, and LSD. In embodiments, the combination comprises tolonidine, 2C-B-FLY, and LP-211. In embodiments, the combination comprises tolonidine, 2C-B-FLY, and LP-44.

In embodiments, the combination comprises tolonidine, 5-MeO-AMT, and 5-CT. In embodiments, the combination comprises tolonidine, 5-MeO-AMT, and 5-MeO-DALT. In embodiments, the combination comprises tolonidine, 5-MeO-AMT, and 5-MeO-DMT. In embodiments, the combination comprises tolonidine, 5-MeO-AMT, and 5-MeO-MiPT. In embodiments, the combination comprises tolonidine, 5-MeO-AMT, and AGH-192. In embodiments, the combination comprises tolonidine, 5-MeO-AMT, and AGH-107. In embodiments, the combination comprises tolonidine, 5-MeO-AMT, and AMT. In embodiments, the combination comprises tolonidine, 5-MeO-AMT, and aripiprazole. In embodiments, the combination comprises tolonidine, 5-MeO-AMT, and AS-19. In embodiments, the combination comprises tolonidine, 5-MeO-AMT, and E-55888. In embodiments, the combination comprises tolonidine, 5-MeO-AMT, and LSD. In embodiments, the combination comprises tolonidine, 5-MeO-AMT, and LP-211. In embodiments, the combination comprises tolonidine, 5-MeO-AMT, and LP-44.

In embodiments, the combination comprises tolonidine, AL-37350A, and 5-CT. In embodiments, the combination comprises tolonidine, AL-37350A, and 5-MeO-DALT. In embodiments, the combination comprises tolonidine, AL-37350A, and 5-MeO-DMT. In embodiments, the combination comprises tolonidine, AL-37350A, and 5-MeO-MiPT In embodiments, the combination comprises tolonidine, AL-37350A, and AGH-192. In embodiments, the combination comprises tolonidine, AL-37350A, and AGH-107. In embodiments, the combination comprises tolonidine, AL-37350A, and AMT. In embodiments, the combination comprises tolonidine, AL-37350A, and aripiprazole. In embodiments, the combination comprises tolonidine, AL-37350A, and AS-19. In embodiments, the combination comprises tolonidine, AL-37350A, and E-55888. In embodiments, the combination comprises tolonidine, AL-37350A, and LSD. In embodiments, the combination comprises tolonidine, AL-37350A, and LP-211. In embodiments, the combination comprises tolonidine, AL-37350A, and LP-44.

In embodiments, the combination comprises tolonidine, CP 809101, and 5-CT. In embodiments, the combination comprises tolonidine, CP 809101, and 5-MeO-DALT. In embodiments, the combination comprises tolonidine, CP 809101, and 5-MeO-DMT. In embodiments, the combination comprises tolonidine, CP 809101, and 5-MeO-MiPT. In embodiments, the combination comprises tolonidine, CP 809101, and AGH-192. In embodiments, the combination comprises tolonidine, CP 809101, and AGH-107. In embodiments, the combination comprises tolonidine, CP 809101, and AMT. In embodiments, the combination comprises tolonidine, CP 809101, and aripiprazole. In embodiments, the combination comprises tolonidine, CP 809101, and AS-19. In embodiments, the combination comprises tolonidine, CP 809101, and E-55888. In embodiments, the combination comprises tolonidine, CP 809101, and LSD. In embodiments, the combination comprises tolonidine, CP 809101, and LP-211. In embodiments, the combination comprises tolonidine, CP 809101, and LP-44.

In embodiments, the combination comprises tolonidine, DALT, and 5-CT. In embodiments, the combination comprises tolonidine, DALT, and 5-MeO-DALT. In embodiments, the combination comprises tolonidine, DALT, and 5-MeO-DMT. In embodiments, the combination comprises tolonidine, DALT, and 5-MeO-MiPT. In embodiments, the combination comprises tolonidine, DALT, and AGH-192. In embodiments, the combination comprises tolonidine, DALT, and AGH-107. In embodiments, the combination comprises tolonidine, DALT, and AMT. In embodiments, the combination comprises tolonidine, DALT, and aripiprazole. In embodiments, the combination comprises tolonidine, DALT, and AS-19. In embodiments, the combination comprises tolonidine, DALT, and E-55888. In embodiments, the combination comprises tolonidine, DALT, and LSD. In embodiments, the combination comprises tolonidine, DALT, and LP-211. In embodiments, the combination comprises tolonidine, DALT, and LP-44.

In embodiments, the combination comprises tolonidine, DOB, and 5-CT. In embodiments, the combination comprises tolonidine, DOB, and 5-MeO-DALT. In embodiments, the combination comprises tolonidine, DOB, and 5-MeO-DMT In embodiments, the combination comprises tolonidine, DOB, and 5-MeO-MiPT. In embodiments, the combination comprises tolonidine, DOB, and AGH-192. In embodiments, the combination comprises tolonidine, DOB, and AGH-107. In embodiments, the combination comprises tolonidine, DOB, and AMT. In embodiments, the combination comprises tolonidine, DOB, and aripiprazole. In embodiments, the combination comprises tolonidine, DOB, and AS-19. In embodiments, the combination comprises tolonidine, DOB, and E-55888. In embodiments, the combination comprises tolonidine, DOB, and LSD. In embodiments, the combination comprises tolonidine, DOB, and LP-211. In embodiments, the combination comprises tolonidine, DOB, and LP-44.

In embodiments, the combination comprises tolonidine, DOET, and 5-CT. In embodiments, the combination comprises tolonidine, DOET, and 5-MeO-DALT. In embodiments, the combination comprises tolonidine, DOET, and 5-MeO-DMT In embodiments, the combination comprises tolonidine, DOET, and 5-MeO-MiPT. In embodiments, the combination comprises tolonidine, DOET, and AGH-192. In embodiments, the combination comprises tolonidine, DOET, and AGH-107. In embodiments, the combination comprises tolonidine, DOET, and AMT. In embodiments, the combination comprises tolonidine, DOET, and aripiprazole. In embodiments, the combination comprises tolonidine, DOET, and AS-19. In embodiments, the combination comprises tolonidine, DOET, and E-55888. In embodiments, the combination comprises tolonidine, DOET, and LSD. In embodiments, the combination comprises tolonidine, DOET, and LP-211. In embodiments, the combination comprises tolonidine, DOET, and LP-44.

In embodiments, the combination comprises tolonidine, DOHx, and 5-CT. In embodiments, the combination comprises tolonidine, DOHx, and 5-MeO-DALT. In embodiments, the combination comprises tolonidine, DOHx, and 5-MeO-DMT. In embodiments, the combination comprises tolonidine, DOHx, and 5-MeO-MiPT. In embodiments, the combination comprises tolonidine, DOHx, and AGH-192. In embodiments, the combination comprises tolonidine, DOHx, and AGH-107. In embodiments, the combination comprises tolonidine, DOHx, and AMT. In embodiments, the combination comprises tolonidine, DOHx, and aripiprazole. In embodiments, the combination comprises tolonidine, DOHx, and AS-19. In embodiments, the combination comprises tolonidine, DOHx, and E-55888. In embodiments, the combination comprises tolonidine, DOHx, and LSD. In embodiments, the combination comprises tolonidine, DOHx, and LP-211. In embodiments, the combination comprises tolonidine, DOHx, and LP-44.

In embodiments, the combination comprises tolonidine, MEM, and 5-CT. In embodiments, the combination comprises tolonidine, MEM, and 5-MeO-DALT. In embodiments, the combination comprises tolonidine, MEM, and 5-MeO-DMT In embodiments, the combination comprises tolonidine, MEM, and 5-MeO-MiPT. In embodiments, the combination comprises tolonidine, MEM, and AGH-192. In embodiments, the combination comprises tolonidine, MEM, and AGH-107. In embodiments, the combination comprises tolonidine, MEM, and AMT. In embodiments, the combination comprises tolonidine, MEM, and aripiprazole. In embodiments, the combination comprises tolonidine, MEM, and AS-19. In embodiments, the combination comprises tolonidine, MEM, and E-55888. In embodiments, the combination comprises tolonidine, MEM, and LSD. In embodiments, the combination comprises tolonidine, MEM, and LP-211. In embodiments, the combination comprises tolonidine, MEM, and LP-44.

In embodiments, the combination comprises tolonidine, CPP, and 5-CT. In embodiments, the combination comprises tolonidine, CPP, and 5-MeO-DALT. In embodiments, the combination comprises tolonidine, CPP, and 5-MeO-DMT. In embodiments, the combination comprises tolonidine, CPP, and 5-MeO-MiPT. In embodiments, the combination comprises tolonidine, CPP, and AGH-192. In embodiments, the combination comprises tolonidine, CPP, and AGH-107. In embodiments, the combination comprises tolonidine, CPP, and AMT. In embodiments, the combination comprises tolonidine, CPP, and aripiprazole. In embodiments, the combination comprises tolonidine, CPP, and AS-19. In embodiments, the combination comprises tolonidine, CPP, and E-55888. In embodiments, the combination comprises tolonidine, CPP, and LSD. In embodiments, the combination comprises tolonidine, CPP, and LP-211. In embodiments, the combination comprises tolonidine, CPP, and LP-44.

In embodiments, the combination comprises tolonidine, NBOH-2C-CN, and 5-CT. In embodiments, the combination comprises tolonidine, NBOH-2C-CN, and 5-MeO-DALT. In embodiments, the combination comprises tolonidine, NBOH-2C-CN, and 5-MeO-DMT. In embodiments, the combination comprises tolonidine, NBOH-2C-CN, and 5-MeO-MiPT In embodiments, the combination comprises tolonidine, NBOH-2C-CN, and AGH-192. In embodiments, the combination comprises tolonidine, NBOH-2C-CN, and AGH-107. In embodiments, the combination comprises tolonidine, NBOH-2C-CN, and AMT. In embodiments, the combination comprises tolonidine, NBOH-2C-CN, and aripiprazole. In embodiments, the combination comprises tolonidine, NBOH-2C-CN, and AS-19. In embodiments, the combination comprises tolonidine, NBOH-2C-CN, and E-55888. In embodiments, the combination comprises tolonidine, NBOH-2C-CN, and LSD. In embodiments, the combination comprises tolonidine, NBOH-2C-CN, and LP-211. In embodiments, the combination comprises tolonidine, NBOH-2C-CN, and LP-44.

In embodiments, the combination comprises tolonidine, TCB-2, and 5-CT. In embodiments, the combination comprises tolonidine, TCB-2, and 5-MeO-DALT. In embodiments, the combination comprises tolonidine, TCB-2, and 5-MeO-DMT In embodiments, the combination comprises tolonidine, TCB-2, and 5-MeO-MiPT. In embodiments, the combination comprises tolonidine, TCB-2, and AGH-192. In embodiments, the combination comprises tolonidine, TCB-2, and AGH-107. In embodiments, the combination comprises tolonidine, TCB-2, and AMT. In embodiments, the combination comprises tolonidine, TCB-2, and aripiprazole. In embodiments, the combination comprises tolonidine, TCB-2, and AS-19. In embodiments, the combination comprises tolonidine, TCB-2, and E-55888. In embodiments, the combination comprises tolonidine, TCB-2, and LSD. In embodiments, the combination comprises tolonidine, TCB-2, and LP-211. In embodiments, the combination comprises tolonidine, TCB-2, and LP-44.

In embodiments, the combination comprises tolonidine, WAY 161503, and 5-CT. In embodiments, the combination comprises tolonidine, WAY 161503, and 5-MeO-DALT. In embodiments, the combination comprises tolonidine, WAY 161503, and 5-MeO-DMT In embodiments, the combination comprises tolonidine, WAY 161503, and 5-MeO-MiPT In embodiments, the combination comprises tolonidine, WAY 161503, and AGH-192. In embodiments, the combination comprises tolonidine, WAY 161503, and AGH-107. In embodiments, the combination comprises tolonidine, WAY 161503, and AMT. In embodiments, the combination comprises tolonidine, WAY 161503, and aripiprazole. In embodiments, the combination comprises tolonidine, WAY 161503, and AS-19. In embodiments, the combination comprises tolonidine, WAY 161503, and E-55888. In embodiments, the combination comprises tolonidine, WAY 161503, and LSD. In embodiments, the combination comprises tolonidine, WAY 161503, and LP-211. In embodiments, the combination comprises tolonidine, WAY 161503, and LP-44.

In embodiments, the combination comprises tizanidine, 1-methylpsilocin, and 5-CT. In embodiments, the combination comprises tizanidine, 1-methylpsilocin, and 5-MeO-DALT. In embodiments, the combination comprises tizanidine, 1-methylpsilocin, and 5-MeO-DMT. In embodiments, the combination comprises tizanidine, 1-methylpsilocin, and 5-MeO-MiPT In embodiments, the combination comprises tizanidine, 1-methylpsilocin, and AGH-192. In embodiments, the combination comprises tizanidine, 1-methylpsilocin, and AGH-107. In embodiments, the combination comprises tizanidine, 1-methylpsilocin, and AMT. In embodiments, the combination comprises tizanidine, 1-methylpsilocin, and aripiprazole. In embodiments, the combination comprises tizanidine, 1-methylpsilocin, and AS-19. In embodiments, the combination comprises tizanidine, 1-methylpsilocin, and E-55888. In embodiments, the combination comprises tizanidine, 1-methylpsilocin, and LSD. In embodiments, the combination comprises tizanidine, 1-methyl psilocin, and LP-211. In embodiments, the combination comprises tizanidine, 1-methylpsilocin, and LP-44.

In embodiments, the combination comprises tizanidine, 2C-B-FLY, and 5-CT. In embodiments, the combination comprises tizanidine, 2C-B-FLY, and 5-MeO-DALT. In embodiments, the combination comprises tizanidine, 2C-B-FLY, and 5-MeO-DMT. In embodiments, the combination comprises tizanidine, 2C-B-FLY, and 5-MeO-MiPT. In embodiments, the combination comprises tizanidine, 2C-B-FLY, and AGH-192. In embodiments, the combination comprises tizanidine, 2C-B-FLY, and AGH-107. In embodiments, the combination comprises tizanidine, 2C-B-FLY, and AMT. In embodiments, the combination comprises tizanidine, 2C-B-FLY, and aripiprazole. In embodiments, the combination comprises tizanidine, 2C-B-FLY, and AS-19. In embodiments, the combination comprises tizanidine, 2C-B-FLY, and E-55888. In embodiments, the combination comprises tizanidine, 2C-B-FLY, and LSD. In embodiments, the combination comprises tizanidine, 2C-B-FLY, and LP-211. In embodiments, the combination comprises tizanidine, 2C-B-FLY, and LP-44.

In embodiments, the combination comprises tizanidine, 5-MeO-AMT, and 5-CT. In embodiments, the combination comprises tizanidine, 5-MeO-AMT, and 5-MeO-DALT. In embodiments, the combination comprises tizanidine, 5-MeO-AMT, and 5-MeO-DMT. In embodiments, the combination comprises tizanidine, 5-MeO-AMT, and 5-MeO-MiPT In embodiments, the combination comprises tizanidine, 5-MeO-AMT, and AGH-192. In embodiments, the combination comprises tizanidine, 5-MeO-AMT, and AGH-107. In embodiments, the combination comprises tizanidine, 5-MeO-AMT, and AMT. In embodiments, the combination comprises tizanidine, 5-MeO-AMT, and aripiprazole. In embodiments, the combination comprises tizanidine, 5-MeO-AMT, and AS-19. In embodiments, the combination comprises tizanidine, 5-MeO-AMT, and E-55888. In embodiments, the combination comprises tizanidine, 5-MeO-AMT, and LSD. In embodiments, the combination comprises tizanidine, 5-MeO-AMT, and LP-211. In embodiments, the combination comprises tizanidine, 5-MeO-AMT, and LP-44.

In embodiments, the combination comprises tizanidine, AL-37350A, and 5-CT. In embodiments, the combination comprises tizanidine, AL-37350A, and 5-MeO-DALT. In embodiments, the combination comprises tizanidine, AL-37350A, and 5-MeO-DMT. In embodiments, the combination comprises tizanidine, AL-37350A, and 5-MeO-MiPT In embodiments, the combination comprises tizanidine, AL-37350A, and AGH-192. In embodiments, the combination comprises tizanidine, AL-37350A, and AGH-107. In embodiments, the combination comprises tizanidine, AL-37350A, and AMT. In embodiments, the combination comprises tizanidine, AL-37350A, and aripiprazole. In embodiments, the combination comprises tizanidine, AL-37350A, and AS-19. In embodiments, the combination comprises tizanidine, AL-37350A, and E-55888. In embodiments, the combination comprises tizanidine, AL-37350A, and LSD. In embodiments, the combination comprises tizanidine, AL-37350A, and LP-211. In embodiments, the combination comprises tizanidine, AL-37350A, and LP-44.

In embodiments, the combination comprises tizanidine, CP 809101, and 5-CT. In embodiments, the combination comprises tizanidine, CP 809101, and 5-MeO-DALT. In embodiments, the combination comprises tizanidine, CP 809101, and 5-MeO-DMT. In embodiments, the combination comprises tizanidine, CP 809101, and 5-MeO-MiPT. In embodiments, the combination comprises tizanidine, CP 809101, and AGH-192. In embodiments, the combination comprises tizanidine, CP 809101, and AGH-107. In embodiments, the combination comprises tizanidine, CP 809101, and AMT. In embodiments, the combination comprises tizanidine, CP 809101, and aripiprazole. In embodiments, the combination comprises tizanidine, CP 809101, and AS-19. In embodiments, the combination comprises tizanidine, CP 809101, and E-55888. In embodiments, the combination comprises tizanidine, CP 809101, and LSD. In embodiments, the combination comprises tizanidine, CP 809101, and LP-211. In embodiments, the combination comprises tizanidine, CP 809101, and LP-44.

In embodiments, the combination comprises tizanidine, DALT, and 5-CT. In embodiments, the combination comprises tizanidine, DALT, and 5-MeO-DALT. In embodiments, the combination comprises tizanidine, DALT, and 5-MeO-DMT In embodiments, the combination comprises tizanidine, DALT, and 5-MeO-MiPT. In embodiments, the combination comprises tizanidine, DALT, and AGH-192. In embodiments, the combination comprises tizanidine, DALT, and AGH-107. In embodiments, the combination comprises tizanidine, DALT, and AMT. In embodiments, the combination comprises tizanidine, DALT, and aripiprazole. In embodiments, the combination comprises tizanidine, DALT, and AS-19. In embodiments, the combination comprises tizanidine, DALT, and E-55888. In embodiments, the combination comprises tizanidine, DALT, and LSD. In embodiments, the combination comprises tizanidine, DALT, and LP-211. In embodiments, the combination comprises tizanidine, DALT, and LP-44.

In embodiments, the combination comprises tizanidine, DOB, and 5-CT. In embodiments, the combination comprises tizanidine, DOB, and 5-MeO-DALT. In embodiments, the combination comprises tizanidine, DOB, and 5-MeO-DMT. In embodiments, the combination comprises tizanidine, DOB, and 5-MeO-MiPT. In embodiments, the combination comprises tizanidine, DOB, and AGH-192. In embodiments, the combination comprises tizanidine, DOB, and AGH-107. In embodiments, the combination comprises tizanidine, DOB, and AMT. In embodiments, the combination comprises tizanidine, DOB, and aripiprazole. In embodiments, the combination comprises tizanidine, DOB, and AS-19. In embodiments, the combination comprises tizanidine, DOB, and E-55888. In embodiments, the combination comprises tizanidine, DOB, and LSD. In embodiments, the combination comprises tizanidine, DOB, and LP-211. In embodiments, the combination comprises tizanidine, DOB, and LP-44.

In embodiments, the combination comprises tizanidine, DOET, and 5-CT. In embodiments, the combination comprises tizanidine, DOET, and 5-MeO-DALT. In embodiments, the combination comprises tizanidine, DOET, and 5-MeO-DMT In embodiments, the combination comprises tizanidine, DOET, and 5-MeO-MiPT. In embodiments, the combination comprises tizanidine, DOET, and AGH-192. In embodiments, the combination comprises tizanidine, DOET, and AGH-107. In embodiments, the combination comprises tizanidine, DOET, and AMT. In embodiments, the combination comprises tizanidine, DOET, and aripiprazole. In embodiments, the combination comprises tizanidine, DOET, and AS-19. In embodiments, the combination comprises tizanidine, DOET, and E-55888. In embodiments, the combination comprises tizanidine, DOET, and LSD. In embodiments, the combination comprises tizanidine, DOET, and LP-211. In embodiments, the combination comprises tizanidine, DOET, and LP-44.

In embodiments, the combination comprises tizanidine, DOHx, and 5-CT. In embodiments, the combination comprises tizanidine, DOHx, and 5-MeO-DALT. In embodiments, the combination comprises tizanidine, DOHx, and 5-MeO-DMT In embodiments, the combination comprises tizanidine, DOHx, and 5-MeO-MiPT. In embodiments, the combination comprises tizanidine, DOHx, and AGH-192. In embodiments, the combination comprises tizanidine, DOHx, and AGH-107. In embodiments, the combination comprises tizanidine, DOHx, and AMT. In embodiments, the combination comprises tizanidine, DOHx, and aripiprazole. In embodiments, the combination comprises tizanidine, DOHx, and AS-19. In embodiments, the combination comprises tizanidine, DOHx, and E-55888. In embodiments, the combination comprises tizanidine, DOHx, and LSD. In embodiments, the combination comprises tizanidine, DOHx, and LP-211. In embodiments, the combination comprises tizanidine, DOHx, and LP-44.

In embodiments, the combination comprises tizanidine, MEM, and 5-CT. In embodiments, the combination comprises tizanidine, MEM, and 5-MeO-DALT. In embodiments, the combination comprises tizanidine, MEM, and 5-MeO-DMT. In embodiments, the combination comprises tizanidine, MEM, and 5-MeO-MiPT. In embodiments, the combination comprises tizanidine, MEM, and AGH-192. In embodiments, the combination comprises tizanidine, MEM, and AGH-107. In embodiments, the combination comprises tizanidine, MEM, and AMT. In embodiments, the combination comprises tizanidine, MEM, and aripiprazole. In embodiments, the combination comprises tizanidine, MEM, and AS-19. In embodiments, the combination comprises tizanidine, MEM, and E-55888. In embodiments, the combination comprises tizanidine, MEM, and LSD. In embodiments, the combination comprises tizanidine, MEM, and LP-211. In embodiments, the combination comprises tizanidine, MEM, and LP-44.

In embodiments, the combination comprises tizanidine, CPP, and 5-CT. In embodiments, the combination comprises tizanidine, CPP, and 5-MeO-DALT. In embodiments, the combination comprises tizanidine, CPP, and 5-MeO-DMT. In embodiments, the combination comprises tizanidine, CPP, and 5-MeO-MiPT. In embodiments, the combination comprises tizanidine, CPP, and AGH-192. In embodiments, the combination comprises tizanidine, CPP, and AGH-107. In embodiments, the combination comprises tizanidine, CPP, and AMT. In embodiments, the combination comprises tizanidine, CPP, and aripiprazole. In embodiments, the combination comprises tizanidine, CPP, and AS-19. In embodiments, the combination comprises tizanidine, CPP, and E-55888. In embodiments, the combination comprises tizanidine, CPP, and LSD. In embodiments, the combination comprises tizanidine, CPP, and LP-211. In embodiments, the combination comprises tizanidine, CPP, and LP-44.

In embodiments, the combination comprises tizanidine, NBOH-2C-CN, and 5-CT. In embodiments, the combination comprises tizanidine, NBOH-2C-CN, and 5-MeO-DALT. In embodiments, the combination comprises tizanidine, NBOH-2C-CN, and 5-MeO-DMT. In embodiments, the combination comprises tizanidine, NBOH-2C-CN, and 5-MeO-MiPT In embodiments, the combination comprises tizanidine, NBOH-2C-CN, and AGH-192. In embodiments, the combination comprises tizanidine, NBOH-2C-CN, and AGH-107. In embodiments, the combination comprises tizanidine, NBOH-2C-CN, and AMT. In embodiments, the combination comprises tizanidine, NBOH-2C-CN, and aripiprazole. In embodiments, the combination comprises tizanidine, NBOH-2C-CN, and AS-19. In embodiments, the combination comprises tizanidine, NBOH-2C-CN, and E-55888. In embodiments, the combination comprises tizanidine, NBOH-2C-CN, and LSD. In embodiments, the combination comprises tizanidine, NBOH-2C-CN, and LP-211. In embodiments, the combination comprises tizanidine, NBOH-2C-CN, and LP-44.

In embodiments, the combination comprises tizanidine, TCB-2, and 5-CT. In embodiments, the combination comprises tizanidine, TCB-2, and 5-MeO-DALT. In embodiments, the combination comprises tizanidine, TCB-2, and 5-MeO-DMT In embodiments, the combination comprises tizanidine, TCB-2, and 5-MeO-MiPT. In embodiments, the combination comprises tizanidine, TCB-2, and AGH-192. In embodiments, the combination comprises tizanidine, TCB-2, and AGH-107. In embodiments, the combination comprises tizanidine, TCB-2, and AMT. In embodiments, the combination comprises tizanidine, TCB-2, and aripiprazole. In embodiments, the combination comprises tizanidine, TCB-2, and AS-19. In embodiments, the combination comprises tizanidine, TCB-2, and E-55888. In embodiments, the combination comprises tizanidine, TCB-2, and LSD. In embodiments, the combination comprises tizanidine, TCB-2, and LP-211. In embodiments, the combination comprises tizanidine, TCB-2, and LP-44.

In embodiments, the combination comprises tizanidine, WAY 161503, and 5-CT. In embodiments, the combination comprises tizanidine, WAY 161503, and 5-MeO-DALT. In embodiments, the combination comprises tizanidine, WAY 161503, and 5-MeO-DMT In embodiments, the combination comprises tizanidine, WAY 161503, and 5-MeO-MiPT. In embodiments, the combination comprises tizanidine, WAY 161503, and AGH-192. In embodiments, the combination comprises tizanidine, WAY 161503, and AGH-107. In embodiments, the combination comprises tizanidine, WAY 161503, and AMT. In embodiments, the combination comprises tizanidine, WAY 161503, and aripiprazole. In embodiments, the combination comprises tizanidine, WAY 161503, and AS-19. In embodiments, the combination comprises tizanidine, WAY 161503, and E-55888. In embodiments, the combination comprises tizanidine, WAY 161503, and LSD. In embodiments, the combination comprises tizanidine, WAY 161503, and LP-211. In embodiments, the combination comprises tizanidine, WAY 161503, and LP-44.

In embodiments, the combination comprises agmatine, a CB₁ agent, 1-methylpsilocin, and 5-CT. In embodiments, the combination comprises agmatine, a CB₁ agent, 1-methylpsilocin, and 5-MeO-DALT. In embodiments, the combination comprises agmatine, a CB₁ agent, 1-methylpsilocin, and 5-MeO-DMT. In embodiments, the combination comprises agmatine, a CB₁ agent, 1-methylpsilocin, and 5-MeO-MiPT In embodiments, the combination comprises agmatine, a CB₁ agent, 1-methylpsilocin, and AGH-192. In embodiments, the combination comprises agmatine, a CB₁ agent, 1-methylpsilocin, and AGH-107. In embodiments, the combination comprises agmatine, a CB₁ agent, 1-methylpsilocin, and AMT. In embodiments, the combination comprises agmatine, a CB₁ agent, 1-methylpsilocin, and aripiprazole. In embodiments, the combination comprises agmatine, a CB₁ agent, 1-methylpsilocin, and AS-19. In embodiments, the combination comprises agmatine, a CB₁ agent, 1-methylpsilocin, and E-55888. In embodiments, the combination comprises agmatine, a CB₁ agent, 1-methylpsilocin, and LSD. In embodiments, the combination comprises agmatine, a CB₁ agent, 1-methylpsilocin, and LP-211. In embodiments, the combination comprises agmatine, a CB₁ agent, 1-methyl psilocin, and LP-44.

In embodiments, the combination comprises agmatine, a CB₁ agent, 2C-B-FLY, and 5-CT. In embodiments, the combination comprises agmatine, a CB₁ agent, 2C-B-FLY, and 5-MeO-DALT. In embodiments, the combination comprises agmatine, a CB₁ agent, 2C-B-FLY, and 5-MeO-DMT In embodiments, the combination comprises agmatine, a CB₁ agent, 2C-B-FLY, and 5-MeO-MiPT. In embodiments, the combination comprises agmatine, a CB₁ agent, 2C-B-FLY, and AGH-192. In embodiments, the combination comprises agmatine, a CB₁ agent, 2C-B-FLY, and AGH-107. In embodiments, the combination comprises agmatine, a CB₁ agent, 2C-B-FLY, and AMT. In embodiments, the combination comprises agmatine, a CB₁ agent, 2C-B-FLY, and aripiprazole. In embodiments, the combination comprises agmatine, a CB₁ agent, 2C-B-FLY, and AS-19. In embodiments, the combination comprises agmatine, a CB₁ agent, 2C-B-FLY, and E-55888. In embodiments, the combination comprises agmatine, a CB₁ agent, 2C-B-FLY, and LSD. In embodiments, the combination comprises agmatine, a CB₁ agent, 2C-B-FLY, and LP-211. In embodiments, the combination comprises agmatine, a CB₁ agent, 2C-B-FLY, and LP-44.

In embodiments, the combination comprises agmatine, a CB₁ agent, 5-MeO-AMT, and 5-CT. In embodiments, the combination comprises agmatine, a CB₁ agent, 5-MeO-AMT, and 5-MeO-DALT. In embodiments, the combination comprises agmatine, a CB₁ agent, 5-MeO-AMT, and 5-MeO-DMT In embodiments, the combination comprises agmatine, a CB₁ agent, 5-MeO-AMT, and 5-MeO-MiPT. In embodiments, the combination comprises agmatine, a CB₁ agent, 5-MeO-AMT, and AGH-192. In embodiments, the combination comprises agmatine, a CB₁ agent, 5-MeO-AMT, and AGH-107. In embodiments, the combination comprises agmatine, a CB₁ agent, 5-MeO-AMT, and AMT. In embodiments, the combination comprises agmatine, a CB₁ agent, 5-MeO-AMT, and aripiprazole. In embodiments, the combination comprises agmatine, a CB₁ agent, 5-MeO-AMT, and AS-19. In embodiments, the combination comprises agmatine, a CB₁ agent, 5-MeO-AMT, and E-55888. In embodiments, the combination comprises agmatine, a CB₁ agent, 5-MeO-AMT, and LSD. In embodiments, the combination comprises agmatine, a CB₁ agent, 5-MeO-AMT, and LP-211. In embodiments, the combination comprises agmatine, a CB₁ agent, 5-MeO-AMT, and LP-44.

In embodiments, the combination comprises agmatine, a CB₁ agent, AL-37350A, and 5-CT. In embodiments, the combination comprises agmatine, a CB₁ agent, AL-37350A, and 5-MeO-DALT. In embodiments, the combination comprises agmatine, a CB₁ agent, AL-37350A, and 5-MeO-DMT In embodiments, the combination comprises agmatine, a CB₁ agent, AL-37350A, and 5-MeO-MiPT. In embodiments, the combination comprises agmatine, a CB₁ agent, AL-37350A, and AGH-192. In embodiments, the combination comprises agmatine, a CB₁ agent, AL-37350A, and AGH-107. In embodiments, the combination comprises agmatine, a CB₁ agent, AL-37350A, and AMT. In embodiments, the combination comprises agmatine, a CB₁ agent, AL-37350A, and aripiprazole. In embodiments, the combination comprises agmatine, a CB₁ agent, AL-37350A, and AS-19. In embodiments, the combination comprises agmatine, a CB₁ agent, AL-37350A, and E-55888. In embodiments, the combination comprises agmatine, a CB₁ agent, AL-37350A, and LSD. In embodiments, the combination comprises agmatine, a CB₁ agent, AL-37350A, and LP-211. In embodiments, the combination comprises agmatine, a CB₁ agent, AL-37350A, and LP-44.

In embodiments, the combination comprises agmatine, a CB₁ agent, CP 809101, and 5-CT. In embodiments, the combination comprises agmatine, a CB₁ agent, CP 809101, and 5-MeO-DALT. In embodiments, the combination comprises agmatine, a CB₁ agent, CP 809101, and 5-MeO-DMT In embodiments, the combination comprises agmatine, a CB₁ agent, CP 809101, and 5-MeO-MiPT. In embodiments, the combination comprises agmatine, a CB₁ agent, CP 809101, and AGH-192. In embodiments, the combination comprises agmatine, a CB₁ agent, CP 809101, and AGH-107. In embodiments, the combination comprises agmatine, a CB₁ agent, CP 809101, and AMT. In embodiments, the combination comprises agmatine, a CB₁ agent, CP 809101, and aripiprazole. In embodiments, the combination comprises agmatine, a CB₁ agent, CP 809101, and AS-19. In embodiments, the combination comprises agmatine, a CB₁ agent, CP 809101, and E-55888. In embodiments, the combination comprises agmatine, a CB₁ agent, CP 809101, and LSD. In embodiments, the combination comprises agmatine, a CB₁ agent, CP 809101, and LP-211. In embodiments, the combination comprises agmatine, a CB₁ agent, CP 809101, and LP-44.

In embodiments, the combination comprises agmatine, a CB₁ agent, DALT, and 5-CT. In embodiments, the combination comprises agmatine, a CB₁ agent, DALT, and 5-MeO-DALT. In embodiments, the combination comprises agmatine, a CB₁ agent, DALT, and 5-MeO-DMT In embodiments, the combination comprises agmatine, a CB₁ agent, DALT, and 5-MeO-MiPT. In embodiments, the combination comprises agmatine, a CB₁ agent, DALT, and AGH-192. In embodiments, the combination comprises agmatine, a CB₁ agent, DALT, and AGH-107. In embodiments, the combination comprises agmatine, a CB₁ agent, DALT, and AMT. In embodiments, the combination comprises agmatine, a CB₁ agent, DALT, and aripiprazole. In embodiments, the combination comprises agmatine, a CB₁ agent, DALT, and AS-19. In embodiments, the combination comprises agmatine, a CB₁ agent, DALT, and E-55888. In embodiments, the combination comprises agmatine, a CB₁ agent, DALT, and LSD. In embodiments, the combination comprises agmatine, a CB₁ agent, DALT, and LP-211. In embodiments, the combination comprises agmatine, a CB₁ agent, DALT, and LP-44.

In embodiments, the combination comprises agmatine, a CB₁ agent, DOB, and 5-CT. In embodiments, the combination comprises agmatine, a CB₁ agent, DOB, and 5-MeO-DALT. In embodiments, the combination comprises agmatine, a CB₁ agent, DOB, and 5-MeO-DMT In embodiments, the combination comprises agmatine, a CB₁ agent, DOB, and 5-MeO-MiPT. In embodiments, the combination comprises agmatine, a CB₁ agent, DOB, and AGH-192. In embodiments, the combination comprises agmatine, a CB₁ agent, DOB, and AGH-107. In embodiments, the combination comprises agmatine, a CB₁ agent, DOB, and AMT. In embodiments, the combination comprises agmatine, a CB₁ agent, DOB, and aripiprazole. In embodiments, the combination comprises agmatine, a CB₁ agent, DOB, and AS-19. In embodiments, the combination comprises agmatine, a CB₁ agent, DOB, and E-55888. In embodiments, the combination comprises agmatine, a CB₁ agent, DOB, and LSD. In embodiments, the combination comprises agmatine, a CB₁ agent, DOB, and LP-211. In embodiments, the combination comprises agmatine, a CB₁ agent, DOB, and LP-44.

In embodiments, the combination comprises agmatine, a CB₁ agent, DOET, and 5-CT. In embodiments, the combination comprises agmatine, a CB₁ agent, DOET, and 5-MeO-DALT. In embodiments, the combination comprises agmatine, a CB₁ agent, DOET, and 5-MeO-DMT. In embodiments, the combination comprises agmatine, a CB₁ agent, DOET, and 5-MeO-MiPT In embodiments, the combination comprises agmatine, a CB₁ agent, DOET, and AGH-192. In embodiments, the combination comprises agmatine, a CB₁ agent, DOET, and AGH-107. In embodiments, the combination comprises agmatine, a CB₁ agent, DOET, and AMT. In embodiments, the combination comprises agmatine, a CB₁ agent, DOET, and aripiprazole. In embodiments, the combination comprises agmatine, a CB₁ agent, DOET, and AS-19. In embodiments, the combination comprises agmatine, a CB₁ agent, DOET, and E-55888. In embodiments, the combination comprises agmatine, a CB₁ agent, DOET, and LSD. In embodiments, the combination comprises agmatine, a CB₁ agent, DOET, and LP-211. In embodiments, the combination comprises agmatine, a CB₁ agent, DOET, and LP-44.

In embodiments, the combination comprises agmatine, a CB₁ agent, DOHx, and 5-CT. In embodiments, the combination comprises agmatine, a CB₁ agent, DOHx, and 5-MeO-DALT. In embodiments, the combination comprises agmatine, a CB₁ agent, DOHx, and 5-MeO-DMT In embodiments, the combination comprises agmatine, a CB₁ agent, DOHx, and 5-MeO-MiPT. In embodiments, the combination comprises agmatine, a CB₁ agent, DOHx, and AGH-192. In embodiments, the combination comprises agmatine, a CB₁ agent, DOHx, and AGH-107. In embodiments, the combination comprises agmatine, a CB₁ agent, DOHx, and AMT. In embodiments, the combination comprises agmatine, a CB₁ agent, DOHx, and aripiprazole. In embodiments, the combination comprises agmatine, a CB₁ agent, DOHx, and AS-19. In embodiments, the combination comprises agmatine, a CB₁ agent, DOHx, and E-55888. In embodiments, the combination comprises agmatine, a CB₁ agent, DOHx, and LSD. In embodiments, the combination comprises agmatine, a CB₁ agent, DOHx, and LP-211. In embodiments, the combination comprises agmatine, a CB₁ agent, DOHx, and LP-44.

In embodiments, the combination comprises agmatine, a CB₁ agent, MEM, and 5-CT. In embodiments, the combination comprises agmatine, a CB₁ agent, MEM, and 5-MeO-DALT. In embodiments, the combination comprises agmatine, a CB₁ agent, MEM, and 5-MeO-DMT. In embodiments, the combination comprises agmatine, a CB₁ agent, MEM, and 5-MeO-MiPT In embodiments, the combination comprises agmatine, a CB₁ agent, MEM, and AGH-192. In embodiments, the combination comprises agmatine, a CB₁ agent, MEM, and AGH-107. In embodiments, the combination comprises agmatine, a CB₁ agent, MEM, and AMT. In embodiments, the combination comprises agmatine, a CB₁ agent, MEM, and aripiprazole. In embodiments, the combination comprises agmatine, a CB₁ agent, MEM, and AS-19. In embodiments, the combination comprises agmatine, a CB₁ agent, MEM, and E-55888. In embodiments, the combination comprises agmatine, a CB₁ agent, MEM, and LSD. In embodiments, the combination comprises agmatine, a CB₁ agent, MEM, and LP-211. In embodiments, the combination comprises agmatine, a CB₁ agent, MEM, and LP-44.

In embodiments, the combination comprises agmatine, a CB₁ agent, CPP, and 5-CT. In embodiments, the combination comprises agmatine, a CB₁ agent, CPP, and 5-MeO-DALT. In embodiments, the combination comprises agmatine, a CB₁ agent, CPP, and 5-MeO-DMT In embodiments, the combination comprises agmatine, a CB₁ agent, CPP, and 5-MeO-MiPT. In embodiments, the combination comprises agmatine, a CB₁ agent, CPP, and AGH-192. In embodiments, the combination comprises agmatine, a CB₁ agent, CPP, and AGH-107. In embodiments, the combination comprises agmatine, a CB₁ agent, CPP, and AMT. In embodiments, the combination comprises agmatine, a CB₁ agent, CPP, and aripiprazole. In embodiments, the combination comprises agmatine, a CB₁ agent, CPP, and AS-19. In embodiments, the combination comprises agmatine, a CB₁ agent, CPP, and E-55888. In embodiments, the combination comprises agmatine, a CB₁ agent, CPP, and LSD. In embodiments, the combination comprises agmatine, a CB₁ agent, CPP, and LP-211. In embodiments, the combination comprises agmatine, a CB₁ agent, CPP, and LP-44.

In embodiments, the combination comprises agmatine, a CB₁ agent, NBOH-2C-CN, and 5-CT. In embodiments, the combination comprises agmatine, a CB₁ agent, NBOH-2C-CN, and 5-MeO-DALT. In embodiments, the combination comprises agmatine, a CB₁ agent, NBOH-2C-CN, and 5-MeO-DMT In embodiments, the combination comprises agmatine, a CB₁ agent, NBOH-2C-CN, and 5-MeO-MiPT. In embodiments, the combination comprises agmatine, a CB₁ agent, NBOH-2C-CN, and AGH-192. In embodiments, the combination comprises agmatine, a CB₁ agent, NBOH-2C-CN, and AGH-107. In embodiments, the combination comprises agmatine, a CB₁ agent, NBOH-2C-CN, and AMT. In embodiments, the combination comprises agmatine, a CB₁ agent, NBOH-2C-CN, and aripiprazole. In embodiments, the combination comprises agmatine, a CB₁ agent, NBOH-2C-CN, and AS-19. In embodiments, the combination comprises agmatine, a CB₁ agent, NBOH-2C-CN, and E-55888. In embodiments, the combination comprises agmatine, a CB₁ agent, NBOH-2C-CN, and LSD. In embodiments, the combination comprises agmatine, a CB₁ agent, NBOH-2C-CN, and LP-211. In embodiments, the combination comprises agmatine, a CB₁ agent, NBOH-2C-CN, and LP-44.

In embodiments, the combination comprises agmatine, a CB₁ agent, TCB-2, and 5-CT. In embodiments, the combination comprises agmatine, a CB₁ agent, TCB-2, and 5-MeO-DALT. In embodiments, the combination comprises agmatine, a CB₁ agent, TCB-2, and 5-MeO-DMT. In embodiments, the combination comprises agmatine, a CB₁ agent, TCB-2, and 5-MeO-MiPT In embodiments, the combination comprises agmatine, a CB₁ agent, TCB-2, and AGH-192. In embodiments, the combination comprises agmatine, a CB₁ agent, TCB-2, and AGH-107. In embodiments, the combination comprises agmatine, a CB₁ agent, TCB-2, and AMT. In embodiments, the combination comprises agmatine, a CB₁ agent, TCB-2, and aripiprazole. In embodiments, the combination comprises agmatine, a CB₁ agent, TCB-2, and AS-19. In embodiments, the combination comprises agmatine, a CB₁ agent, TCB-2, and E-55888. In embodiments, the combination comprises agmatine, a CB₁ agent, TCB-2, and LSD. In embodiments, the combination comprises agmatine, a CB₁ agent, TCB-2, and LP-211. In embodiments, the combination comprises agmatine, a CB₁ agent, TCB-2, and LP-44.

In embodiments, the combination comprises agmatine, a CB₁ agent, WAY 161503, and 5-CT. In embodiments, the combination comprises agmatine, a CB₁ agent, WAY 161503, and 5-MeO-DALT. In embodiments, the combination comprises agmatine, a CB₁ agent, WAY 161503, and 5-MeO-DMT. In embodiments, the combination comprises agmatine, a CB₁ agent, WAY 161503, and 5-MeO-MiPT. In embodiments, the combination comprises agmatine, a CB₁ agent, WAY 161503, and AGH-192. In embodiments, the combination comprises agmatine, a CB₁ agent, WAY 161503, and AGH-107. In embodiments, the combination comprises agmatine, a CB₁ agent, WAY 161503, and AMT. In embodiments, the combination comprises agmatine, a CB₁ agent, WAY 161503, and aripiprazole. In embodiments, the combination comprises agmatine, a CB₁ agent, WAY 161503, and AS-19. In embodiments, the combination comprises agmatine, a CB₁ agent, WAY 161503, and E-55888. In embodiments, the combination comprises agmatine, a CB₁ agent, WAY 161503, and LSD. In embodiments, the combination comprises agmatine, a CB₁ agent, WAY 161503, and LP-211. In embodiments, the combination comprises agmatine, a CB₁ agent, WAY 161503, and LP-44.

In embodiments, the combination comprises BDBM50091347, a CB₁ agent, 1-methylpsilocin, and 5-CT. In embodiments, the combination comprises BDBM50091347, a CB₁ agent, 1-methylpsilocin, and 5-MeO-DALT. In embodiments, the combination comprises BDBM50091347, a CB₁ agent, 1-methylpsilocin, and 5-MeO-DMT. In embodiments, the combination comprises BDBM50091347, a CB₁ agent, 1-methylpsilocin, and 5-MeO-MiPT In embodiments, the combination comprises BDBM50091347, a CB₁ agent, 1-methyl psilocin, and AGH-192. In embodiments, the combination comprises BDBM50091347, a CB₁ agent, 1-methyl psilocin, and AGH-107. In embodiments, the combination comprises BDBM50091347, a CB₁ agent, 1-methylpsilocin, and AMT. In embodiments, the combination comprises BDBM50091347, a CB₁ agent, 1-methylpsilocin, and aripiprazole. In embodiments, the combination comprises BDBM50091347, a CB₁ agent, 1-methylpsilocin, and AS-19. In embodiments, the combination comprises BDBM50091347, a CB₁ agent, 1-methylpsilocin, and E-55888. In embodiments, the combination comprises BDBM50091347, a CB₁ agent, 1-methylpsilocin, and LSD. In embodiments, the combination comprises BDBM50091347, a CB₁ agent, 1-methylpsilocin, and LP-211. In embodiments, the combination comprises BDBM50091347, a CB₁ agent, 1-methylpsilocin, and LP-44.

In embodiments, the combination comprises BDBM50091347, a CB₁ agent, 2C-B-FLY, and 5-CT. In embodiments, the combination comprises BDBM50091347, a CB₁ agent, 2C-B-FLY, and 5-MeO-DALT. In embodiments, the combination comprises BDBM50091347, a CB₁ agent, 2C-B-FLY, and 5-MeO-DMT. In embodiments, the combination comprises BDBM50091347, a CB₁ agent, 2C-B-FLY, and 5-MeO-MiPT In embodiments, the combination comprises BDBM50091347, a CB₁ agent, 2C-B-FLY, and AGH-192. In embodiments, the combination comprises BDBM50091347, a CB₁ agent, 2C-B-FLY, and AGH-107. In embodiments, the combination comprises BDBM50091347, a CB₁ agent, 2C-B-FLY, and AMT. In embodiments, the combination comprises BDBM50091347, a CB₁ agent, 2C-B-FLY, and aripiprazole. In embodiments, the combination comprises BDBM50091347, a CB₁ agent, 2C-B-FLY, and AS-19. In embodiments, the combination comprises BDBM50091347, a CB₁ agent, 2C-B-FLY, and E-55888. In embodiments, the combination comprises BDBM50091347, a CB₁ agent, 2C-B-FLY, and LSD. In embodiments, the combination comprises BDBM50091347, a CB₁ agent, 2C-B-FLY, and LP-211. In embodiments, the combination comprises BDBM50091347, a CB₁ agent, 2C-B-FLY, and LP-44.

In embodiments, the combination comprises BDBM50091347, a CB₁ agent, 5-MeO-AMT, and 5-CT. In embodiments, the combination comprises BDBM50091347, a CB₁ agent, 5-MeO-AMT, and 5-MeO-DALT. In embodiments, the combination comprises BDBM50091347, a CB₁ agent, 5-MeO-AMT, and 5-MeO-DMT. In embodiments, the combination comprises BDBM50091347, a CB₁ agent, 5-MeO-AMT, and 5-MeO-MiPT. In embodiments, the combination comprises BDBM50091347, a CB₁ agent, 5-MeO-AMT, and AGH-192. In embodiments, the combination comprises BDBM50091347, a CB₁ agent, 5-MeO-AMT, and AGH-107. In embodiments, the combination comprises BDBM50091347, a CB₁ agent, 5-MeO-AMT, and AMT. In embodiments, the combination comprises BDBM50091347, a CB₁ agent, 5-MeO-AMT, and aripiprazole. In embodiments, the combination comprises BDBM50091347, a CB₁ agent, 5-MeO-AMT, and AS-19. In embodiments, the combination comprises BDBM50091347, a CB₁ agent, 5-MeO-AMT, and E-55888. In embodiments, the combination comprises BDBM50091347, a CB₁ agent, 5-MeO-AMT, and LSD. In embodiments, the combination comprises BDBM50091347, a CB₁ agent, 5-MeO-AMT, and LP-211. In embodiments, the combination comprises BDBM50091347, a CB₁ agent, 5-MeO-AMT, and LP-44.

In embodiments, the combination comprises BDBM50091347, a CB₁ agent, AL-37350A, and 5-CT. In embodiments, the combination comprises BDBM50091347, a CB₁ agent, AL-37350A, and 5-MeO-DALT. In embodiments, the combination comprises BDBM50091347, a CB₁ agent, AL-37350A, and 5-MeO-DMT. In embodiments, the combination comprises BDBM50091347, a CB₁ agent, AL-37350A, and 5-MeO-MiPT. In embodiments, the combination comprises BDBM50091347, a CB₁ agent, AL-37350A, and AGH-192. In embodiments, the combination comprises BDBM50091347, a CB₁ agent, AL-37350A, and AGH-107. In embodiments, the combination comprises BDBM50091347, a CB₁ agent, AL-37350A, and AMT. In embodiments, the combination comprises BDBM50091347, a CB₁ agent, AL-37350A, and aripiprazole. In embodiments, the combination comprises BDBM50091347, a CB₁ agent, AL-37350A, and AS-19. In embodiments, the combination comprises BDBM50091347, a CB₁ agent, AL-37350A, and E-55888. In embodiments, the combination comprises BDBM50091347, a CB₁ agent, AL-37350A, and LSD. In embodiments, the combination comprises BDBM50091347, a CB₁ agent, AL-37350A, and LP-211. In embodiments, the combination comprises BDBM50091347, a CB₁ agent, AL-37350A, and LP-44.

In embodiments, the combination comprises BDBM50091347, a CB₁ agent, CP 809101, and 5-CT. In embodiments, the combination comprises BDBM50091347, a CB₁ agent, CP 809101, and 5-MeO-DALT. In embodiments, the combination comprises BDBM50091347, a CB₁ agent, CP 809101, and 5-MeO-DMT. In embodiments, the combination comprises BDBM50091347, a CB₁ agent, CP 809101, and 5-MeO-MiPT. In embodiments, the combination comprises BDBM50091347, a CB₁ agent, CP 809101, and AGH-192. In embodiments, the combination comprises BDBM50091347, a CB₁ agent, CP 809101, and AGH-107. In embodiments, the combination comprises BDBM50091347, a CB₁ agent, CP 809101, and AMT. In embodiments, the combination comprises BDBM50091347, a CB₁ agent, CP 809101, and aripiprazole. In embodiments, the combination comprises BDBM50091347, a CB₁ agent, CP 809101, and AS-19. In embodiments, the combination comprises BDBM50091347, a CB₁ agent, CP 809101, and E-55888. In embodiments, the combination comprises BDBM50091347, a CB₁ agent, CP 809101, and LSD. In embodiments, the combination comprises BDBM50091347, a CB₁ agent, CP 809101, and LP-211. In embodiments, the combination comprises BDBM50091347, a CB₁ agent, CP 809101, and LP-44.

In embodiments, the combination comprises BDBM50091347, a CB₁ agent, DALT, and 5-CT. In embodiments, the combination comprises BDBM50091347, a CB₁ agent, DALT, and 5-MeO-DALT. In embodiments, the combination comprises BDBM50091347, a CB₁ agent, DALT, and 5-MeO-DMT. In embodiments, the combination comprises BDBM50091347, a CB₁ agent, DALT, and 5-MeO-MiPT In embodiments, the combination comprises BDBM50091347, a CB₁ agent, DALT, and AGH-192. In embodiments, the combination comprises BDBM50091347, a CB₁ agent, DALT, and AGH-107. In embodiments, the combination comprises BDBM50091347, a CB₁ agent, DALT, and AMT. In embodiments, the combination comprises BDBM50091347, a CB₁ agent, DALT, and aripiprazole. In embodiments, the combination comprises BDBM50091347, a CB₁ agent, DALT, and AS-19. In embodiments, the combination comprises BDBM50091347, a CB₁ agent, DALT, and E-55888. In embodiments, the combination comprises BDBM50091347, a CB₁ agent, DALT, and LSD. In embodiments, the combination comprises BDBM50091347, a CB₁ agent, DALT, and LP-211. In embodiments, the combination comprises BDBM50091347, a CB₁ agent, DALT, and LP-44.

In embodiments, the combination comprises BDBM50091347, a CB₁ agent, DOB, and 5-CT. In embodiments, the combination comprises BDBM50091347, a CB₁ agent, DOB, and 5-MeO-DALT. In embodiments, the combination comprises BDBM50091347, a CB₁ agent, DOB, and 5-MeO-DMT. In embodiments, the combination comprises BDBM50091347, a CB₁ agent, DOB, and 5-MeO-MiPT. In embodiments, the combination comprises BDBM50091347, a CB₁ agent, DOB, and AGH-192. In embodiments, the combination comprises BDBM50091347, a CB₁ agent, DOB, and AGH-107. In embodiments, the combination comprises BDBM50091347, a CB₁ agent, DOB, and AMT. In embodiments, the combination comprises BDBM50091347, a CB₁ agent, DOB, and aripiprazole. In embodiments, the combination comprises BDBM50091347, a CB₁ agent, DOB, and AS-19. In embodiments, the combination comprises BDBM50091347, a CB₁ agent, DOB, and E-55888. In embodiments, the combination comprises BDBM50091347, a CB₁ agent, DOB, and LSD. In embodiments, the combination comprises BDBM50091347, a CB₁ agent, DOB, and LP-211. In embodiments, the combination comprises BDBM50091347, a CB₁ agent, DOB, and LP-44.

In embodiments, the combination comprises BDBM50091347, a CB₁ agent, DOET, and 5-CT. In embodiments, the combination comprises BDBM50091347, a CB₁ agent, DOET, and 5-MeO-DALT. In embodiments, the combination comprises BDBM50091347, a CB₁ agent, DOET, and 5-MeO-DMT In embodiments, the combination comprises BDBM50091347, a CB₁ agent, DOET, and 5-MeO-MiPT. In embodiments, the combination comprises BDBM50091347, a CB₁ agent, DOET, and AGH-192. In embodiments, the combination comprises BDBM50091347, a CB₁ agent, DOET, and AGH-107. In embodiments, the combination comprises BDBM50091347, a CB₁ agent, DOET, and AMT. In embodiments, the combination comprises BDBM50091347, a CB₁ agent, DOET, and aripiprazole. In embodiments, the combination comprises BDBM50091347, a CB₁ agent, DOET, and AS-19. In embodiments, the combination comprises BDBM50091347, a CB₁ agent, DOET, and E-55888. In embodiments, the combination comprises BDBM50091347, a CB₁ agent, DOET, and LSD. In embodiments, the combination comprises BDBM50091347, a CB₁ agent, DOET, and LP-211. In embodiments, the combination comprises BDBM50091347, a CB₁ agent, DOET, and LP-44.

In embodiments, the combination comprises BDBM50091347, a CB₁ agent, DOHx, and 5-CT. In embodiments, the combination comprises BDBM50091347, a CB₁ agent, DOHx, and 5-MeO-DALT. In embodiments, the combination comprises BDBM50091347, a CB₁ agent, DOHx, and 5-MeO-DMT In embodiments, the combination comprises BDBM50091347, a CB₁ agent, DOHx, and 5-MeO-MiPT. In embodiments, the combination comprises BDBM50091347, a CB₁ agent, DOHx, and AGH-192. In embodiments, the combination comprises BDBM50091347, a CB₁ agent, DOHx, and AGH-107. In embodiments, the combination comprises BDBM50091347, a CB₁ agent, DOHx, and AMT. In embodiments, the combination comprises BDBM50091347, a CB₁ agent, DOHx, and aripiprazole. In embodiments, the combination comprises BDBM50091347, a CB₁ agent, DOHx, and AS-19. In embodiments, the combination comprises BDBM50091347, a CB₁ agent, DOHx, and E-55888. In embodiments, the combination comprises BDBM50091347, a CB₁ agent, DOHx, and LSD. In embodiments, the combination comprises BDBM50091347, a CB₁ agent, DOHx, and LP-211. In embodiments, the combination comprises BDBM50091347, a CB₁ agent, DOHx, and LP-44.

In embodiments, the combination comprises BDBM50091347, a CB₁ agent, MEM, and 5-CT. In embodiments, the combination comprises BDBM50091347, a CB₁ agent, MEM, and 5-MeO-DALT. In embodiments, the combination comprises BDBM50091347, a CB₁ agent, MEM, and 5-MeO-DMT. In embodiments, the combination comprises BDBM50091347, a CB₁ agent, MEM, and 5-MeO-MiPT. In embodiments, the combination comprises BDBM50091347, a CB₁ agent, MEM, and AGH-192. In embodiments, the combination comprises BDBM50091347, a CB₁ agent, MEM, and AGH-107. In embodiments, the combination comprises BDBM50091347, a CB₁ agent, MEM, and AMT. In embodiments, the combination comprises BDBM50091347, a CB₁ agent, MEM, and aripiprazole. In embodiments, the combination comprises BDBM50091347, a CB₁ agent, MEM, and AS-19. In embodiments, the combination comprises BDBM50091347, a CB₁ agent, MEM, and E-55888. In embodiments, the combination comprises BDBM50091347, a CB₁ agent, MEM, and LSD. In embodiments, the combination comprises BDBM50091347, a CB₁ agent, MEM, and LP-211. In embodiments, the combination comprises BDBM50091347, a CB₁ agent, MEM, and LP-44.

In embodiments, the combination comprises BDBM50091347, a CB₁ agent, CPP, and 5-CT. In embodiments, the combination comprises BDBM50091347, a CB₁ agent, CPP, and 5-MeO-DALT. In embodiments, the combination comprises BDBM50091347, a CB₁ agent, CPP, and 5-MeO-DMT In embodiments, the combination comprises BDBM50091347, a CB₁ agent, CPP, and 5-MeO-MiPT. In embodiments, the combination comprises BDBM50091347, a CB₁ agent, CPP, and AGH-192. In embodiments, the combination comprises BDBM50091347, a CB₁ agent, CPP, and AGH-107. In embodiments, the combination comprises BDBM50091347, a CB₁ agent, CPP, and AMT. In embodiments, the combination comprises BDBM50091347, a CB₁ agent, CPP, and aripiprazole. In embodiments, the combination comprises BDBM50091347, a CB₁ agent, CPP, and AS-19. In embodiments, the combination comprises BDBM50091347, a CB₁ agent, CPP, and E-55888. In embodiments, the combination comprises BDBM50091347, a CB₁ agent, CPP, and LSD. In embodiments, the combination comprises BDBM50091347, a CB₁ agent, CPP, and LP-211. In embodiments, the combination comprises BDBM50091347, a CB₁ agent, CPP, and LP-44.

In embodiments, the combination comprises BDBM50091347, a CB₁ agent, NBOH-2C-CN, and 5-CT. In embodiments, the combination comprises BDBM50091347, a CB₁ agent, NBOH-2C-CN, and 5-MeO-DALT. In embodiments, the combination comprises BDBM50091347, a CB₁ agent, NBOH-2C-CN, and 5-MeO-DMT. In embodiments, the combination comprises BDBM50091347, a CB₁ agent, NBOH-2C-CN, and 5-MeO-MiPT. In embodiments, the combination comprises BDBM50091347, a CB₁ agent, NBOH-2C-CN, and AGH-192. In embodiments, the combination comprises BDBM50091347, a CB₁ agent, NBOH-2C-CN, and AGH-107. In embodiments, the combination comprises BDBM50091347, a CB₁ agent, NBOH-2C-CN, and AMT. In embodiments, the combination comprises BDBM50091347, a CB₁ agent, NBOH-2C-CN, and aripiprazole. In embodiments, the combination comprises BDBM50091347, a CB₁ agent, NBOH-2C-CN, and AS-19. In embodiments, the combination comprises BDBM50091347, a CB₁ agent, NBOH-2C-CN, and E-55888. In embodiments, the combination comprises BDBM50091347, a CB₁ agent, NBOH-2C-CN, and LSD. In embodiments, the combination comprises BDBM50091347, a CB₁ agent, NBOH-2C-CN, and LP-211. In embodiments, the combination comprises BDBM50091347, a CB₁ agent, NBOH-2C-CN, and LP-44.

In embodiments, the combination comprises BDBM50091347, a CB₁ agent, TCB-2, and 5-CT. In embodiments, the combination comprises BDBM50091347, a CB₁ agent, TCB-2, and 5-MeO-DALT. In embodiments, the combination comprises BDBM50091347, a CB₁ agent, TCB-2, and 5-MeO-DMT. In embodiments, the combination comprises BDBM50091347, a CB₁ agent, TCB-2, and 5-MeO-MiPT. In embodiments, the combination comprises BDBM50091347, a CB₁ agent, TCB-2, and AGH-192. In embodiments, the combination comprises BDBM50091347, a CB₁ agent, TCB-2, and AGH-107. In embodiments, the combination comprises BDBM50091347, a CB₁ agent, TCB-2, and AMT. In embodiments, the combination comprises BDBM50091347, a CB₁ agent, TCB-2, and aripiprazole. In embodiments, the combination comprises BDBM50091347, a CB₁ agent, TCB-2, and AS-19. In embodiments, the combination comprises BDBM50091347, a CB₁ agent, TCB-2, and E-55888. In embodiments, the combination comprises BDBM50091347, a CB₁ agent, TCB-2, and LSD. In embodiments, the combination comprises BDBM50091347, a CB₁ agent, TCB-2, and LP-211. In embodiments, the combination comprises BDBM50091347, a CB₁ agent, TCB-2, and LP-44.

In embodiments, the combination comprises BDBM50091347, a CB₁ agent, WAY 161503, and 5-CT. In embodiments, the combination comprises BDBM50091347, a CB₁ agent, WAY 161503, and 5-MeO-DALT. In embodiments, the combination comprises BDBM50091347, a CB₁ agent, WAY 161503, and 5-MeO-DMT. In embodiments, the combination comprises BDBM50091347, a CB₁ agent, WAY 161503, and 5-MeO-MiPT. In embodiments, the combination comprises BDBM50091347, a CB₁ agent, WAY 161503, and AGH-192. In embodiments, the combination comprises BDBM50091347, a CB₁ agent, WAY 161503, and AGH-107. In embodiments, the combination comprises BDBM50091347, a CB₁ agent, WAY 161503, and AMT. In embodiments, the combination comprises BDBM50091347, a CB₁ agent, WAY 161503, and aripiprazole. In embodiments, the combination comprises BDBM50091347, a CB₁ agent, WAY 161503, and AS-19. In embodiments, the combination comprises BDBM50091347, a CB₁ agent, WAY 161503, and E-55888. In embodiments, the combination comprises BDBM50091347, a CB₁ agent, WAY 161503, and LSD. In embodiments, the combination comprises BDBM50091347, a CB₁ agent, WAY 161503, and LP-211. In embodiments, the combination comprises BDBM50091347, a CB₁ agent, WAY 161503, and LP-44.

In embodiments, the combination comprises clonidine, a CB₁ agent, 1-methylpsilocin, and 5-CT. In embodiments, the combination comprises clonidine, a CB₁ agent, 1-methylpsilocin, and 5-MeO-DALT. In embodiments, the combination comprises clonidine, a CB₁ agent, 1-methylpsilocin, and 5-MeO-DMT In embodiments, the combination comprises clonidine, a CB₁ agent, 1-methylpsilocin, and 5-MeO-MiPT. In embodiments, the combination comprises clonidine, a CB₁ agent, 1-methylpsilocin, and AGH-192. In embodiments, the combination comprises clonidine, a CB₁ agent, 1-methylpsilocin, and AGH-107. In embodiments, the combination comprises clonidine, a CB₁ agent, 1-methylpsilocin, and AMT. In embodiments, the combination comprises clonidine, a CB₁ agent, 1-methylpsilocin, and aripiprazole. In embodiments, the combination comprises clonidine, a CB₁ agent, 1-methylpsilocin, and AS-19. In embodiments, the combination comprises clonidine, a CB₁ agent, 1-methylpsilocin, and E-55888. In embodiments, the combination comprises clonidine, a CB₁ agent, 1-methylpsilocin, and LSD. In embodiments, the combination comprises clonidine, a CB₁ agent, 1-methylpsilocin, and LP-211. In embodiments, the combination comprises clonidine, a CB₁ agent, 1-methylpsilocin, and LP-44.

In embodiments, the combination comprises clonidine, a CB₁ agent, 2C-B-FLY, and 5-CT. In embodiments, the combination comprises clonidine, a CB₁ agent, 2C-B-FLY, and 5-MeO-DALT. In embodiments, the combination comprises clonidine, a CB₁ agent, 2C-B-FLY, and 5-MeO-DMT In embodiments, the combination comprises clonidine, a CB₁ agent, 2C-B-FLY, and 5-MeO-MiPT In embodiments, the combination comprises clonidine, a CB₁ agent, 2C-B-FLY, and AGH-192. In embodiments, the combination comprises clonidine, a CB₁ agent, 2C-B-FLY, and AGH-107. In embodiments, the combination comprises clonidine, a CB₁ agent, 2C-B-FLY, and AMT. In embodiments, the combination comprises clonidine, a CB₁ agent, 2C-B-FLY, and aripiprazole. In embodiments, the combination comprises clonidine, a CB₁ agent, 2C-B-FLY, and AS-19. In embodiments, the combination comprises clonidine, a CB₁ agent, 2C-B-FLY, and E-55888. In embodiments, the combination comprises clonidine, a CB₁ agent, 2C-B-FLY, and LSD. In embodiments, the combination comprises clonidine, a CB₁ agent, 2C-B-FLY, and LP-211. In embodiments, the combination comprises clonidine, a CB₁ agent, 2C-B-FLY, and LP-44.

In embodiments, the combination comprises clonidine, a CB₁ agent, 5-MeO-AMT, and 5-CT. In embodiments, the combination comprises clonidine, a CB₁ agent, 5-MeO-AMT, and 5-MeO-DALT. In embodiments, the combination comprises clonidine, a CB₁ agent, 5-MeO-AMT, and 5-MeO-DMT. In embodiments, the combination comprises clonidine, a CB₁ agent, 5-MeO-AMT, and 5-MeO-MiPT In embodiments, the combination comprises clonidine, a CB₁ agent, 5-MeO-AMT, and AGH-192. In embodiments, the combination comprises clonidine, a CB₁ agent, 5-MeO-AMT, and AGH-107. In embodiments, the combination comprises clonidine, a CB₁ agent, 5-MeO-AMT, and AMT. In embodiments, the combination comprises clonidine, a CB₁ agent, 5-MeO-AMT, and aripiprazole. In embodiments, the combination comprises clonidine, a CB₁ agent, 5-MeO-AMT, and AS-19. In embodiments, the combination comprises clonidine, a CB₁ agent, 5-MeO-AMT, and E-55888. In embodiments, the combination comprises clonidine, a CB₁ agent, 5-MeO-AMT, and LSD. In embodiments, the combination comprises clonidine, a CB₁ agent, 5-MeO-AMT, and LP-211. In embodiments, the combination comprises clonidine, a CB₁ agent, 5-MeO-AMT, and LP-44.

In embodiments, the combination comprises clonidine, a CB₁ agent, AL-37350A, and 5-CT. In embodiments, the combination comprises clonidine, a CB₁ agent, AL-37350A, and 5-MeO-DALT. In embodiments, the combination comprises clonidine, a CB₁ agent, AL-37350A, and 5-MeO-DMT. In embodiments, the combination comprises clonidine, a CB₁ agent, AL-37350A, and 5-MeO-MiPT In embodiments, the combination comprises clonidine, a CB₁ agent, AL-37350A, and AGH-192. In embodiments, the combination comprises clonidine, a CB₁ agent, AL-37350A, and AGH-107. In embodiments, the combination comprises clonidine, a CB₁ agent, AL-37350A, and AMT. In embodiments, the combination comprises clonidine, a CB₁ agent, AL-37350A, and aripiprazole. In embodiments, the combination comprises clonidine, a CB₁ agent, AL-37350A, and AS-19. In embodiments, the combination comprises clonidine, a CB₁ agent, AL-37350A, and E-55888. In embodiments, the combination comprises clonidine, a CB₁ agent, AL-37350A, and LSD. In embodiments, the combination comprises clonidine, a CB₁ agent, AL-37350A, and LP-211. In embodiments, the combination comprises clonidine, a CB₁ agent, AL-37350A, and LP-44.

In embodiments, the combination comprises clonidine, a CB₁ agent, CP 809101, and 5-CT. In embodiments, the combination comprises clonidine, a CB₁ agent, CP 809101, and 5-MeO-DALT. In embodiments, the combination comprises clonidine, a CB₁ agent, CP 809101, and 5-MeO-DMT. In embodiments, the combination comprises clonidine, a CB₁ agent, CP 809101, and 5-MeO-MiPT In embodiments, the combination comprises clonidine, a CB₁ agent, CP 809101, and AGH-192. In embodiments, the combination comprises clonidine, a CB₁ agent, CP 809101, and AGH-107. In embodiments, the combination comprises clonidine, a CB₁ agent, CP 809101, and AMT. In embodiments, the combination comprises clonidine, a CB₁ agent, CP 809101, and aripiprazole. In embodiments, the combination comprises clonidine, a CB₁ agent, CP 809101, and AS-19. In embodiments, the combination comprises clonidine, a CB₁ agent, CP 809101, and E-55888. In embodiments, the combination comprises clonidine, a CB₁ agent, CP 809101, and LSD. In embodiments, the combination comprises clonidine, a CB₁ agent, CP 809101, and LP-211. In embodiments, the combination comprises clonidine, a CB₁ agent, CP 809101, and LP-44.

In embodiments, the combination comprises clonidine, a CB₁ agent, DALT, and 5-CT. In embodiments, the combination comprises clonidine, a CB₁ agent, DALT, and 5-MeO-DALT. In embodiments, the combination comprises clonidine, a CB₁ agent, DALT, and 5-MeO-DMT. In embodiments, the combination comprises clonidine, a CB₁ agent, DALT, and 5-MeO-MiPT. In embodiments, the combination comprises clonidine, a CB₁ agent, DALT, and AGH-192. In embodiments, the combination comprises clonidine, a CB₁ agent, DALT, and AGH-107. In embodiments, the combination comprises clonidine, a CB₁ agent, DALT, and AMT. In embodiments, the combination comprises clonidine, a CB₁ agent, DALT, and aripiprazole. In embodiments, the combination comprises clonidine, a CB₁ agent, DALT, and AS-19. In embodiments, the combination comprises clonidine, a CB₁ agent, DALT, and E-55888. In embodiments, the combination comprises clonidine, a CB₁ agent, DALT, and LSD. In embodiments, the combination comprises clonidine, a CB₁ agent, DALT, and LP-211. In embodiments, the combination comprises clonidine, a CB₁ agent, DALT, and LP-44.

In embodiments, the combination comprises clonidine, a CB₁ agent, DOB, and 5-CT. In embodiments, the combination comprises clonidine, a CB₁ agent, DOB, and 5-MeO-DALT. In embodiments, the combination comprises clonidine, a CB₁ agent, DOB, and 5-MeO-DMT In embodiments, the combination comprises clonidine, a CB₁ agent, DOB, and 5-MeO-MiPT In embodiments, the combination comprises clonidine, a CB₁ agent, DOB, and AGH-192. In embodiments, the combination comprises clonidine, a CB₁ agent, DOB, and AGH-107. In embodiments, the combination comprises clonidine, a CB₁ agent, DOB, and AMT. In embodiments, the combination comprises clonidine, a CB₁ agent, DOB, and aripiprazole. In embodiments, the combination comprises clonidine, a CB₁ agent, DOB, and AS-19. In embodiments, the combination comprises clonidine, a CB₁ agent, DOB, and E-55888. In embodiments, the combination comprises clonidine, a CB₁ agent, DOB, and LSD. In embodiments, the combination comprises clonidine, a CB₁ agent, DOB, and LP-211. In embodiments, the combination comprises clonidine, a CB₁ agent, DOB, and LP-44.

In embodiments, the combination comprises clonidine, a CB₁ agent, DOET, and 5-CT. In embodiments, the combination comprises clonidine, a CB₁ agent, DOET, and 5-MeO-DALT. In embodiments, the combination comprises clonidine, a CB₁ agent, DOET, and 5-MeO-DMT In embodiments, the combination comprises clonidine, a CB₁ agent, DOET, and 5-MeO-MiPT. In embodiments, the combination comprises clonidine, a CB₁ agent, DOET, and AGH-192. In embodiments, the combination comprises clonidine, a CB₁ agent, DOET, and AGH-107. In embodiments, the combination comprises clonidine, a CB₁ agent, DOET, and AMT. In embodiments, the combination comprises clonidine, a CB₁ agent, DOET, and aripiprazole. In embodiments, the combination comprises clonidine, a CB₁ agent, DOET, and AS-19. In embodiments, the combination comprises clonidine, a CB₁ agent, DOET, and E-55888. In embodiments, the combination comprises clonidine, a CB₁ agent, DOET, and LSD. In embodiments, the combination comprises clonidine, a CB₁ agent, DOET, and LP-211. In embodiments, the combination comprises clonidine, a CB₁ agent, DOET, and LP-44.

In embodiments, the combination comprises clonidine, a CB₁ agent, DOHx, and 5-CT. In embodiments, the combination comprises clonidine, a CB₁ agent, DOHx, and 5-MeO-DALT. In embodiments, the combination comprises clonidine, a CB₁ agent, DOHx, and 5-MeO-DMT In embodiments, the combination comprises clonidine, a CB₁ agent, DOHx, and 5-MeO-MiPT In embodiments, the combination comprises clonidine, a CB₁ agent, DOHx, and AGH-192. In embodiments, the combination comprises clonidine, a CB₁ agent, DOHx, and AGH-107. In embodiments, the combination comprises clonidine, a CB₁ agent, DOHx, and AMT. In embodiments, the combination comprises clonidine, a CB₁ agent, DOHx, and aripiprazole. In embodiments, the combination comprises clonidine, a CB₁ agent, DOHx, and AS-19. In embodiments, the combination comprises clonidine, a CB₁ agent, DOHx, and E-55888. In embodiments, the combination comprises clonidine, a CB₁ agent, DOHx, and LSD. In embodiments, the combination comprises clonidine, a CB₁ agent, DOHx, and LP-211. In embodiments, the combination comprises clonidine, a CB₁ agent, DOHx, and LP-44.

In embodiments, the combination comprises clonidine, a CB₁ agent, MEM, and 5-CT. In embodiments, the combination comprises clonidine, a CB₁ agent, MEM, and 5-MeO-DALT. In embodiments, the combination comprises clonidine, a CB₁ agent, MEM, and 5-MeO-DMT In embodiments, the combination comprises clonidine, a CB₁ agent, MEM, and 5-MeO-MiPT. In embodiments, the combination comprises clonidine, a CB₁ agent, MEM, and AGH-192. In embodiments, the combination comprises clonidine, a CB₁ agent, MEM, and AGH-107. In embodiments, the combination comprises clonidine, a CB₁ agent, MEM, and AMT. In embodiments, the combination comprises clonidine, a CB₁ agent, MEM, and aripiprazole. In embodiments, the combination comprises clonidine, a CB₁ agent, MEM, and AS-19. In embodiments, the combination comprises clonidine, a CB₁ agent, MEM, and E-55888. In embodiments, the combination comprises clonidine, a CB₁ agent, MEM, and LSD. In embodiments, the combination comprises clonidine, a CB₁ agent, MEM, and LP-211. In embodiments, the combination comprises clonidine, a CB₁ agent, MEM, and LP-44.

In embodiments, the combination comprises clonidine, a CB₁ agent, CPP, and 5-CT. In embodiments, the combination comprises clonidine, a CB₁ agent, CPP, and 5-MeO-DALT. In embodiments, the combination comprises clonidine, a CB₁ agent, CPP, and 5-MeO-DMT. In embodiments, the combination comprises clonidine, a CB₁ agent, CPP, and 5-MeO-MiPT. In embodiments, the combination comprises clonidine, a CB₁ agent, CPP, and AGH-192. In embodiments, the combination comprises clonidine, a CB₁ agent, CPP, and AGH-107. In embodiments, the combination comprises clonidine, a CB₁ agent, CPP, and AMT. In embodiments, the combination comprises clonidine, a CB₁ agent, CPP, and aripiprazole. In embodiments, the combination comprises clonidine, a CB₁ agent, CPP, and AS-19. In embodiments, the combination comprises clonidine, a CB₁ agent, CPP, and E-55888. In embodiments, the combination comprises clonidine, a CB₁ agent, CPP, and LSD. In embodiments, the combination comprises clonidine, a CB₁ agent, CPP, and LP-211. In embodiments, the combination comprises clonidine, a CB₁ agent, CPP, and LP-44.

In embodiments, the combination comprises clonidine, a CB₁ agent, NBOH-2C-CN, and 5-CT. In embodiments, the combination comprises clonidine, a CB₁ agent, NBOH-2C-CN, and 5-MeO-DALT. In embodiments, the combination comprises clonidine, a CB₁ agent, NBOH-2C-CN, and 5-MeO-DMT. In embodiments, the combination comprises clonidine, a CB₁ agent, NBOH-2C-CN, and 5-MeO-MiPT In embodiments, the combination comprises clonidine, a CB₁ agent, NBOH-2C-CN, and AGH-192. In embodiments, the combination comprises clonidine, a CB₁ agent, NBOH-2C-CN, and AGH-107. In embodiments, the combination comprises clonidine, a CB₁ agent, NBOH-2C-CN, and AMT. In embodiments, the combination comprises clonidine, a CB₁ agent, NBOH-2C-CN, and aripiprazole. In embodiments, the combination comprises clonidine, a CB₁ agent, NBOH-2C-CN, and AS-19. In embodiments, the combination comprises clonidine, a CB₁ agent, NBOH-2C-CN, and E-55888. In embodiments, the combination comprises clonidine, a CB₁ agent, NBOH-2C-CN, and LSD. In embodiments, the combination comprises clonidine, a CB₁ agent, NBOH-2C-CN, and LP-211. In embodiments, the combination comprises clonidine, a CB₁ agent, NBOH-2C-CN, and LP-44.

In embodiments, the combination comprises clonidine, a CB₁ agent, TCB-2, and 5-CT. In embodiments, the combination comprises clonidine, a CB₁ agent, TCB-2, and 5-MeO-DALT. In embodiments, the combination comprises clonidine, a CB₁ agent, TCB-2, and 5-MeO-DMT In embodiments, the combination comprises clonidine, a CB₁ agent, TCB-2, and 5-MeO-MiPT. In embodiments, the combination comprises clonidine, a CB₁ agent, TCB-2, and AGH-192. In embodiments, the combination comprises clonidine, a CB₁ agent, TCB-2, and AGH-107. In embodiments, the combination comprises clonidine, a CB₁ agent, TCB-2, and AMT. In embodiments, the combination comprises clonidine, a CB₁ agent, TCB-2, and aripiprazole. In embodiments, the combination comprises clonidine, a CB₁ agent, TCB-2, and AS-19. In embodiments, the combination comprises clonidine, a CB₁ agent, TCB-2, and E-55888. In embodiments, the combination comprises clonidine, a CB₁ agent, TCB-2, and LSD. In embodiments, the combination comprises clonidine, a CB₁ agent, TCB-2, and LP-211. In embodiments, the combination comprises clonidine, a CB₁ agent, TCB-2, and LP-44.

In embodiments, the combination comprises clonidine, a CB₁ agent, WAY 161503, and 5-CT. In embodiments, the combination comprises clonidine, a CB₁ agent, WAY 161503, and 5-MeO-DALT. In embodiments, the combination comprises clonidine, a CB₁ agent, WAY 161503, and 5-MeO-DMT. In embodiments, the combination comprises clonidine, a CB₁ agent, WAY 161503, and 5-MeO-MiPT. In embodiments, the combination comprises clonidine, a CB₁ agent, WAY 161503, and AGH-192. In embodiments, the combination comprises clonidine, a CB₁ agent, WAY 161503, and AGH-107. In embodiments, the combination comprises clonidine, a CB₁ agent, WAY 161503, and AMT. In embodiments, the combination comprises clonidine, a CB₁ agent, WAY 161503, and aripiprazole. In embodiments, the combination comprises clonidine, a CB₁ agent, WAY 161503, and AS-19. In embodiments, the combination comprises clonidine, a CB₁ agent, WAY 161503, and E-55888. In embodiments, the combination comprises clonidine, a CB₁ agent, WAY 161503, and LSD. In embodiments, the combination comprises clonidine, a CB₁ agent, WAY 161503, and LP-211. In embodiments, the combination comprises clonidine, a CB₁ agent, WAY 161503, and LP-44.

In embodiments, the combination comprises guanfacine, a CB₁ agent, 1-methylpsilocin, and 5-CT. In embodiments, the combination comprises guanfacine, a CB₁ agent, 1-methylpsilocin, and 5-MeO-DALT. In embodiments, the combination comprises guanfacine, a CB₁ agent, 1-methylpsilocin, and 5-MeO-DMT In embodiments, the combination comprises guanfacine, a CB₁ agent, 1-methylpsilocin, and 5-MeO-MiPT In embodiments, the combination comprises guanfacine, a CB₁ agent, 1-methylpsilocin, and AGH-192. In embodiments, the combination comprises guanfacine, a CB₁ agent, 1-methylpsilocin, and AGH-107. In embodiments, the combination comprises guanfacine, a CB₁ agent, 1-methylpsilocin, and AMT. In embodiments, the combination comprises guanfacine, a CB₁ agent, 1-methylpsilocin, and aripiprazole. In embodiments, the combination comprises guanfacine, a CB₁ agent, 1-methylpsilocin, and AS-19. In embodiments, the combination comprises guanfacine, a CB₁ agent, 1-methylpsilocin, and E-55888. In embodiments, the combination comprises guanfacine, a CB₁ agent, 1-methylpsilocin, and LSD. In embodiments, the combination comprises guanfacine, a CB₁ agent, 1-methylpsilocin, and LP-211. In embodiments, the combination comprises guanfacine, a CB₁ agent, 1-methyl psilocin, and LP-44.

In embodiments, the combination comprises guanfacine, a CB₁ agent, 2C-B-FLY, and 5-CT. In embodiments, the combination comprises guanfacine, a CB₁ agent, 2C-B-FLY, and 5-MeO-DALT. In embodiments, the combination comprises guanfacine, a CB₁ agent, 2C-B-FLY, and 5-MeO-DMT. In embodiments, the combination comprises guanfacine, a CB₁ agent, 2C-B-FLY, and 5-MeO-MiPT. In embodiments, the combination comprises guanfacine, a CB₁ agent, 2C-B-FLY, and AGH-192. In embodiments, the combination comprises guanfacine, a CB₁ agent, 2C-B-FLY, and AGH-107. In embodiments, the combination comprises guanfacine, a CB₁ agent, 2C-B-FLY, and AMT. In embodiments, the combination comprises guanfacine, a CB₁ agent, 2C-B-FLY, and aripiprazole. In embodiments, the combination comprises guanfacine, a CB₁ agent, 2C-B-FLY, and AS-19. In embodiments, the combination comprises guanfacine, a CB₁ agent, 2C-B-FLY, and E-55888. In embodiments, the combination comprises guanfacine, a CB₁ agent, 2C-B-FLY, and LSD. In embodiments, the combination comprises guanfacine, a CB₁ agent, 2C-B-FLY, and LP-211. In embodiments, the combination comprises guanfacine, a CB₁ agent, 2C-B-FLY, and LP-44.

In embodiments, the combination comprises guanfacine, a CB₁ agent, 5-MeO-AMT, and 5-CT. In embodiments, the combination comprises guanfacine, a CB₁ agent, 5-MeO-AMT, and 5-MeO-DALT. In embodiments, the combination comprises guanfacine, a CB₁ agent, 5-MeO-AMT, and 5-MeO-DMT In embodiments, the combination comprises guanfacine, a CB₁ agent, 5-MeO-AMT, and 5-MeO-MiPT. In embodiments, the combination comprises guanfacine, a CB₁ agent, 5-MeO-AMT, and AGH-192. In embodiments, the combination comprises guanfacine, a CB₁ agent, 5-MeO-AMT, and AGH-107. In embodiments, the combination comprises guanfacine, a CB₁ agent, 5-MeO-AMT, and AMT. In embodiments, the combination comprises guanfacine, a CB₁ agent, 5-MeO-AMT, and aripiprazole. In embodiments, the combination comprises guanfacine, a CB₁ agent, 5-MeO-AMT, and AS-19. In embodiments, the combination comprises guanfacine, a CB₁ agent, 5-MeO-AMT, and E-55888. In embodiments, the combination comprises guanfacine, a CB₁ agent, 5-MeO-AMT, and LSD. In embodiments, the combination comprises guanfacine, a CB₁ agent, 5-MeO-AMT, and LP-211. In embodiments, the combination comprises guanfacine, a CB₁ agent, 5-MeO-AMT, and LP-44.

In embodiments, the combination comprises guanfacine, a CB₁ agent, AL-37350A, and 5-CT. In embodiments, the combination comprises guanfacine, a CB₁ agent, AL-37350A, and 5-MeO-DALT. In embodiments, the combination comprises guanfacine, a CB₁ agent, AL-37350A, and 5-MeO-DMT. In embodiments, the combination comprises guanfacine, a CB₁ agent, AL-37350A, and 5-MeO-MiPT. In embodiments, the combination comprises guanfacine, a CB₁ agent, AL-37350A, and AGH-192. In embodiments, the combination comprises guanfacine, a CB₁ agent, AL-37350A, and AGH-107. In embodiments, the combination comprises guanfacine, a CB₁ agent, AL-37350A, and AMT. In embodiments, the combination comprises guanfacine, a CB₁ agent, AL-37350A, and aripiprazole. In embodiments, the combination comprises guanfacine, a CB₁ agent, AL-37350A, and AS-19. In embodiments, the combination comprises guanfacine, a CB₁ agent, AL-37350A, and E-55888. In embodiments, the combination comprises guanfacine, a CB₁ agent, AL-37350A, and LSD. In embodiments, the combination comprises guanfacine, a CB₁ agent, AL-37350A, and LP-211. In embodiments, the combination comprises guanfacine, a CB₁ agent, AL-37350A, and LP-44.

In embodiments, the combination comprises guanfacine, a CB₁ agent, CP 809101, and 5-CT. In embodiments, the combination comprises guanfacine, a CB₁ agent, CP 809101, and 5-MeO-DALT. In embodiments, the combination comprises guanfacine, a CB₁ agent, CP 809101, and 5-MeO-DMT. In embodiments, the combination comprises guanfacine, a CB₁ agent, CP 809101, and 5-MeO-MiPT. In embodiments, the combination comprises guanfacine, a CB₁ agent, CP 809101, and AGH-192. In embodiments, the combination comprises guanfacine, a CB₁ agent, CP 809101, and AGH-107. In embodiments, the combination comprises guanfacine, a CB₁ agent, CP 809101, and AMT. In embodiments, the combination comprises guanfacine, a CB₁ agent, CP 809101, and aripiprazole. In embodiments, the combination comprises guanfacine, a CB₁ agent, CP 809101, and AS-19. In embodiments, the combination comprises guanfacine, a CB₁ agent, CP 809101, and E-55888. In embodiments, the combination comprises guanfacine, a CB₁ agent, CP 809101, and LSD. In embodiments, the combination comprises guanfacine, a CB₁ agent, CP 809101, and LP-211. In embodiments, the combination comprises guanfacine, a CB₁ agent, CP 809101, and LP-44.

In embodiments, the combination comprises guanfacine, a CB₁ agent, DALT, and 5-CT. In embodiments, the combination comprises guanfacine, a CB₁ agent, DALT, and 5-MeO-DALT. In embodiments, the combination comprises guanfacine, a CB₁ agent, DALT, and 5-MeO-DMT. In embodiments, the combination comprises guanfacine, a CB₁ agent, DALT, and 5-MeO-MiPT. In embodiments, the combination comprises guanfacine, a CB₁ agent, DALT, and AGH-192. In embodiments, the combination comprises guanfacine, a CB₁ agent, DALT, and AGH-107. In embodiments, the combination comprises guanfacine, a CB₁ agent, DALT, and AMT. In embodiments, the combination comprises guanfacine, a CB₁ agent, DALT, and aripiprazole. In embodiments, the combination comprises guanfacine, a CB₁ agent, DALT, and AS-19. In embodiments, the combination comprises guanfacine, a CB₁ agent, DALT, and E-55888. In embodiments, the combination comprises guanfacine, a CB₁ agent, DALT, and LSD. In embodiments, the combination comprises guanfacine, a CB₁ agent, DALT, and LP-211. In embodiments, the combination comprises guanfacine, a CB₁ agent, DALT, and LP-44.

In embodiments, the combination comprises guanfacine, a CB₁ agent, DOB, and 5-CT. In embodiments, the combination comprises guanfacine, a CB₁ agent, DOB, and 5-MeO-DALT. In embodiments, the combination comprises guanfacine, a CB₁ agent, DOB, and 5-MeO-DMT. In embodiments, the combination comprises guanfacine, a CB₁ agent, DOB, and 5-MeO-MiPT In embodiments, the combination comprises guanfacine, a CB₁ agent, DOB, and AGH-192. In embodiments, the combination comprises guanfacine, a CB₁ agent, DOB, and AGH-107. In embodiments, the combination comprises guanfacine, a CB₁ agent, DOB, and AMT. In embodiments, the combination comprises guanfacine, a CB₁ agent, DOB, and aripiprazole. In embodiments, the combination comprises guanfacine, a CB₁ agent, DOB, and AS-19. In embodiments, the combination comprises guanfacine, a CB₁ agent, DOB, and E-55888. In embodiments, the combination comprises guanfacine, a CB₁ agent, DOB, and LSD. In embodiments, the combination comprises guanfacine, a CB₁ agent, DOB, and LP-211. In embodiments, the combination comprises guanfacine, a CB₁ agent, DOB, and LP-44.

In embodiments, the combination comprises guanfacine, a CB₁ agent, DOET, and 5-CT. In embodiments, the combination comprises guanfacine, a CB₁ agent, DOET, and 5-MeO-DALT. In embodiments, the combination comprises guanfacine, a CB₁ agent, DOET, and 5-MeO-DMT. In embodiments, the combination comprises guanfacine, a CB₁ agent, DOET, and 5-MeO-MiPT In embodiments, the combination comprises guanfacine, a CB₁ agent, DOET, and AGH-192. In embodiments, the combination comprises guanfacine, a CB₁ agent, DOET, and AGH-107. In embodiments, the combination comprises guanfacine, a CB₁ agent, DOET, and AMT. In embodiments, the combination comprises guanfacine, a CB₁ agent, DOET, and aripiprazole. In embodiments, the combination comprises guanfacine, a CB₁ agent, DOET, and AS-19. In embodiments, the combination comprises guanfacine, a CB₁ agent, DOET, and E-55888. In embodiments, the combination comprises guanfacine, a CB₁ agent, DOET, and LSD. In embodiments, the combination comprises guanfacine, a CB₁ agent, DOET, and LP-211. In embodiments, the combination comprises guanfacine, a CB₁ agent, DOET, and LP-44.

In embodiments, the combination comprises guanfacine, a CB₁ agent, DOHx, and 5-CT. In embodiments, the combination comprises guanfacine, a CB₁ agent, DOHx, and 5-MeO-DALT. In embodiments, the combination comprises guanfacine, a CB₁ agent, DOHx, and 5-MeO-DMT. In embodiments, the combination comprises guanfacine, a CB₁ agent, DOHx, and 5-MeO-MiPT In embodiments, the combination comprises guanfacine, a CB₁ agent, DOHx, and AGH-192. In embodiments, the combination comprises guanfacine, a CB₁ agent, DOHx, and AGH-107. In embodiments, the combination comprises guanfacine, a CB₁ agent, DOHx, and AMT. In embodiments, the combination comprises guanfacine, a CB₁ agent, DOHx, and aripiprazole. In embodiments, the combination comprises guanfacine, a CB₁ agent, DOHx, and AS-19. In embodiments, the combination comprises guanfacine, a CB₁ agent, DOHx, and E-55888. In embodiments, the combination comprises guanfacine, a CB₁ agent, DOHx, and LSD. In embodiments, the combination comprises guanfacine, a CB₁ agent, DOHx, and LP-211. In embodiments, the combination comprises guanfacine, a CB₁ agent, DOHx, and LP-44.

In embodiments, the combination comprises guanfacine, a CB₁ agent, MEM, and 5-CT. In embodiments, the combination comprises guanfacine, a CB₁ agent, MEM, and 5-MeO-DALT. In embodiments, the combination comprises guanfacine, a CB₁ agent, MEM, and 5-MeO-DMT In embodiments, the combination comprises guanfacine, a CB₁ agent, MEM, and 5-MeO-MiPT In embodiments, the combination comprises guanfacine, a CB₁ agent, MEM, and AGH-192. In embodiments, the combination comprises guanfacine, a CB₁ agent, MEM, and AGH-107. In embodiments, the combination comprises guanfacine, a CB₁ agent, MEM, and AMT. In embodiments, the combination comprises guanfacine, a CB₁ agent, MEM, and aripiprazole. In embodiments, the combination comprises guanfacine, a CB₁ agent, MEM, and AS-19. In embodiments, the combination comprises guanfacine, a CB₁ agent, MEM, and E-55888. In embodiments, the combination comprises guanfacine, a CB₁ agent, MEM, and LSD. In embodiments, the combination comprises guanfacine, a CB₁ agent, MEM, and LP-211. In embodiments, the combination comprises guanfacine, a CB₁ agent, MEM, and LP-44.

In embodiments, the combination comprises guanfacine, a CB₁ agent, CPP, and 5-CT. In embodiments, the combination comprises guanfacine, a CB₁ agent, CPP, and 5-MeO-DALT. In embodiments, the combination comprises guanfacine, a CB₁ agent, CPP, and 5-MeO-DMT In embodiments, the combination comprises guanfacine, a CB₁ agent, CPP, and 5-MeO-MiPT In embodiments, the combination comprises guanfacine, a CB₁ agent, CPP, and AGH-192. In embodiments, the combination comprises guanfacine, a CB₁ agent, CPP, and AGH-107. In embodiments, the combination comprises guanfacine, a CB₁ agent, CPP, and AMT. In embodiments, the combination comprises guanfacine, a CB₁ agent, CPP, and aripiprazole. In embodiments, the combination comprises guanfacine, a CB₁ agent, CPP, and AS-19. In embodiments, the combination comprises guanfacine, a CB₁ agent, CPP, and E-55888. In embodiments, the combination comprises guanfacine, a CB₁ agent, CPP, and LSD. In embodiments, the combination comprises guanfacine, a CB₁ agent, CPP, and LP-211. In embodiments, the combination comprises guanfacine, a CB₁ agent, CPP, and LP-44.

In embodiments, the combination comprises guanfacine, a CB₁ agent, NBOH-2C-CN, and 5-CT. In embodiments, the combination comprises guanfacine, a CB₁ agent, NBOH-2C-CN, and 5-MeO-DALT. In embodiments, the combination comprises guanfacine, a CB₁ agent, NBOH-2C-CN, and 5-MeO-DMT In embodiments, the combination comprises guanfacine, a CB₁ agent, NBOH-2C-CN, and 5-MeO-MiPT. In embodiments, the combination comprises guanfacine, a CB₁ agent, NBOH-2C-CN, and AGH-192. In embodiments, the combination comprises guanfacine, a CB₁ agent, NBOH-2C-CN, and AGH-107. In embodiments, the combination comprises guanfacine, a CB₁ agent, NBOH-2C-CN, and AMT. In embodiments, the combination comprises guanfacine, a CB₁ agent, NBOH-2C-CN, and aripiprazole. In embodiments, the combination comprises guanfacine, a CB₁ agent, NBOH-2C-CN, and AS-19. In embodiments, the combination comprises guanfacine, a CB₁ agent, NBOH-2C-CN, and E-55888. In embodiments, the combination comprises guanfacine, a CB₁ agent, NBOH-2C-CN, and LSD. In embodiments, the combination comprises guanfacine, a CB₁ agent, NBOH-2C-CN, and LP-211. In embodiments, the combination comprises guanfacine, a CB₁ agent, NBOH-2C-CN, and LP-44.

In embodiments, the combination comprises guanfacine, a CB₁ agent, TCB-2, and 5-CT. In embodiments, the combination comprises guanfacine, a CB₁ agent, TCB-2, and 5-MeO-DALT. In embodiments, the combination comprises guanfacine, a CB₁ agent, TCB-2, and 5-MeO-DMT In embodiments, the combination comprises guanfacine, a CB₁ agent, TCB-2, and 5-MeO-MiPT. In embodiments, the combination comprises guanfacine, a CB₁ agent, TCB-2, and AGH-192. In embodiments, the combination comprises guanfacine, a CB₁ agent, TCB-2, and AGH-107. In embodiments, the combination comprises guanfacine, a CB₁ agent, TCB-2, and AMT. In embodiments, the combination comprises guanfacine, a CB₁ agent, TCB-2, and aripiprazole. In embodiments, the combination comprises guanfacine, a CB₁ agent, TCB-2, and AS-19. In embodiments, the combination comprises guanfacine, a CB₁ agent, TCB-2, and E-55888. In embodiments, the combination comprises guanfacine, a CB₁ agent, TCB-2, and LSD. In embodiments, the combination comprises guanfacine, a CB₁ agent, TCB-2, and LP-211. In embodiments, the combination comprises guanfacine, a CB₁ agent, TCB-2, and LP-44.

In embodiments, the combination comprises guanfacine, a CB₁ agent, WAY 161503, and 5-CT. In embodiments, the combination comprises guanfacine, a CB₁ agent, WAY 161503, and 5-MeO-DALT. In embodiments, the combination comprises guanfacine, a CB₁ agent, WAY 161503, and 5-MeO-DMT. In embodiments, the combination comprises guanfacine, a CB₁ agent, WAY 161503, and 5-MeO-MiPT In embodiments, the combination comprises guanfacine, a CB₁ agent, WAY 161503, and AGH-192. In embodiments, the combination comprises guanfacine, a CB₁ agent, WAY 161503, and AGH-107. In embodiments, the combination comprises guanfacine, a CB₁ agent, WAY 161503, and AMT. In embodiments, the combination comprises guanfacine, a CB₁ agent, WAY 161503, and aripiprazole. In embodiments, the combination comprises guanfacine, a CB₁ agent, WAY 161503, and AS-19. In embodiments, the combination comprises guanfacine, a CB₁ agent, WAY 161503, and E-55888. In embodiments, the combination comprises guanfacine, a CB₁ agent, WAY 161503, and LSD. In embodiments, the combination comprises guanfacine, a CB₁ agent, WAY 161503, and LP-211. In embodiments, the combination comprises guanfacine, a CB₁ agent, WAY 161503, and LP-44.

In embodiments, the combination comprises mCPP, a CB₁ agent, 1-methylpsilocin, and 5-CT. In embodiments, the combination comprises mCPP, a CB₁ agent, 1-methylpsilocin, and 5-MeO-DALT. In embodiments, the combination comprises mCPP, a CB₁ agent, 1-methylpsilocin, and 5-MeO-DMT In embodiments, the combination comprises mCPP, a CB₁ agent, 1-methylpsilocin, and 5-MeO-MiPT. In embodiments, the combination comprises mCPP, a CB₁ agent, 1-methylpsilocin, and AGH-192. In embodiments, the combination comprises mCPP, a CB₁ agent, 1-methylpsilocin, and AGH-107. In embodiments, the combination comprises mCPP, a CB₁ agent, 1-methylpsilocin, and AMT. In embodiments, the combination comprises mCPP, a CB₁ agent, 1-methylpsilocin, and aripiprazole. In embodiments, the combination comprises mCPP, a CB₁ agent, 1-methylpsilocin, and AS-19. In embodiments, the combination comprises mCPP, a CB₁ agent, 1-methylpsilocin, and E-55888. In embodiments, the combination comprises mCPP, a CB₁ agent, 1-methylpsilocin, and LSD. In embodiments, the combination comprises mCPP, a CB₁ agent, 1-methylpsilocin, and LP-211. In embodiments, the combination comprises mCPP, a CB₁ agent, 1-methylpsilocin, and LP-44.

In embodiments, the combination comprises mCPP, a CB₁ agent, 2C-B-FLY, and 5-CT. In embodiments, the combination comprises mCPP, a CB₁ agent, 2C-B-FLY, and 5-MeO-DALT. In embodiments, the combination comprises mCPP, a CB₁ agent, 2C-B-FLY, and 5-MeO-DMT. In embodiments, the combination comprises mCPP, a CB₁ agent, 2C-B-FLY, and 5-MeO-MiPT In embodiments, the combination comprises mCPP, a CB₁ agent, 2C-B-FLY, and AGH-192. In embodiments, the combination comprises mCPP, a CB₁ agent, 2C-B-FLY, and AGH-107. In embodiments, the combination comprises mCPP, a CB₁ agent, 2C-B-FLY, and AMT. In embodiments, the combination comprises mCPP, a CB₁ agent, 2C-B-FLY, and aripiprazole. In embodiments, the combination comprises mCPP, a CB₁ agent, 2C-B-FLY, and AS-19. In embodiments, the combination comprises mCPP, a CB₁ agent, 2C-B-FLY, and E-55888. In embodiments, the combination comprises mCPP, a CB₁ agent, 2C-B-FLY, and LSD. In embodiments, the combination comprises mCPP, a CB₁ agent, 2C-B-FLY, and LP-211. In embodiments, the combination comprises mCPP, a CB₁ agent, 2C-B-FLY, and LP-44.

In embodiments, the combination comprises mCPP, a CB₁ agent, 5-MeO-AMT, and 5-CT. In embodiments, the combination comprises mCPP, a CB₁ agent, 5-MeO-AMT, and 5-MeO-DALT. In embodiments, the combination comprises mCPP, a CB₁ agent, 5-MeO-AMT, and 5-MeO-DMT. In embodiments, the combination comprises mCPP, a CB₁ agent, 5-MeO-AMT, and 5-MeO-MiPT. In embodiments, the combination comprises mCPP, a CB₁ agent, 5-MeO-AMT, and AGH-192. In embodiments, the combination comprises mCPP, a CB₁ agent, 5-MeO-AMT, and AGH-107. In embodiments, the combination comprises mCPP, a CB₁ agent, 5-MeO-AMT, and AMT. In embodiments, the combination comprises mCPP, a CB₁ agent, 5-MeO-AMT, and aripiprazole. In embodiments, the combination comprises mCPP, a CB₁ agent, 5-MeO-AMT, and AS-19. In embodiments, the combination comprises mCPP, a CB₁ agent, 5-MeO-AMT, and E-55888. In embodiments, the combination comprises mCPP, a CB₁ agent, 5-MeO-AMT, and LSD. In embodiments, the combination comprises mCPP, a CB₁ agent, 5-MeO-AMT, and LP-211. In embodiments, the combination comprises mCPP, a CB₁ agent, 5-MeO-AMT, and LP-44.

In embodiments, the combination comprises mCPP, a CB₁ agent, AL-37350A, and 5-CT. In embodiments, the combination comprises mCPP, a CB₁ agent, AL-37350A, and 5-MeO-DALT. In embodiments, the combination comprises mCPP, a CB₁ agent, AL-37350A, and 5-MeO-DMT. In embodiments, the combination comprises mCPP, a CB₁ agent, AL-37350A, and 5-MeO-MiPT In embodiments, the combination comprises mCPP, a CB₁ agent, AL-37350A, and AGH-192. In embodiments, the combination comprises mCPP, a CB₁ agent, AL-37350A, and AGH-107. In embodiments, the combination comprises mCPP, a CB₁ agent, AL-37350A, and AMT. In embodiments, the combination comprises mCPP, a CB₁ agent, AL-37350A, and aripiprazole. In embodiments, the combination comprises mCPP, a CB₁ agent, AL-37350A, and AS-19. In embodiments, the combination comprises mCPP, a CB₁ agent, AL-37350A, and E-55888. In embodiments, the combination comprises mCPP, a CB₁ agent, AL-37350A, and LSD. In embodiments, the combination comprises mCPP, a CB₁ agent, AL-37350A, and LP-211. In embodiments, the combination comprises mCPP, a CB₁ agent, AL-37350A, and LP-44.

In embodiments, the combination comprises mCPP, a CB₁ agent, CP 809101, and 5-CT. In embodiments, the combination comprises mCPP, a CB₁ agent, CP 809101, and 5-MeO-DALT. In embodiments, the combination comprises mCPP, a CB₁ agent, CP 809101, and 5-MeO-DMT. In embodiments, the combination comprises mCPP, a CB₁ agent, CP 809101, and 5-MeO-MiPT In embodiments, the combination comprises mCPP, a CB₁ agent, CP 809101, and AGH-192. In embodiments, the combination comprises mCPP, a CB₁ agent, CP 809101, and AGH-107. In embodiments, the combination comprises mCPP, a CB₁ agent, CP 809101, and AMT. In embodiments, the combination comprises mCPP, a CB₁ agent, CP 809101, and aripiprazole. In embodiments, the combination comprises mCPP, a CB₁ agent, CP 809101, and AS-19. In embodiments, the combination comprises mCPP, a CB₁ agent, CP 809101, and E-55888. In embodiments, the combination comprises mCPP, a CB₁ agent, CP 809101, and LSD. In embodiments, the combination comprises mCPP, a CB₁ agent, CP 809101, and LP-211. In embodiments, the combination comprises mCPP, a CB₁ agent, CP 809101, and LP-44.

In embodiments, the combination comprises mCPP, a CB₁ agent, DALT, and 5-CT. In embodiments, the combination comprises mCPP, a CB₁ agent, DALT, and 5-MeO-DALT. In embodiments, the combination comprises mCPP, a CB₁ agent, DALT, and 5-MeO-DMT In embodiments, the combination comprises mCPP, a CB₁ agent, DALT, and 5-MeO-MiPT. In embodiments, the combination comprises mCPP, a CB₁ agent, DALT, and AGH-192. In embodiments, the combination comprises mCPP, a CB₁ agent, DALT, and AGH-107. In embodiments, the combination comprises mCPP, a CB₁ agent, DALT, and AMT. In embodiments, the combination comprises mCPP, a CB₁ agent, DALT, and aripiprazole. In embodiments, the combination comprises mCPP, a CB₁ agent, DALT, and AS-19. In embodiments, the combination comprises mCPP, a CB₁ agent, DALT, and E-55888. In embodiments, the combination comprises mCPP, a CB₁ agent, DALT, and LSD. In embodiments, the combination comprises mCPP, a CB₁ agent, DALT, and LP-211. In embodiments, the combination comprises mCPP, a CB₁ agent, DALT, and LP-44.

In embodiments, the combination comprises mCPP, a CB₁ agent, DOB, and 5-CT. In embodiments, the combination comprises mCPP, a CB₁ agent, DOB, and 5-MeO-DALT. In embodiments, the combination comprises mCPP, a CB₁ agent, DOB, and 5-MeO-DMT. In embodiments, the combination comprises mCPP, a CB₁ agent, DOB, and 5-MeO-MiPT In embodiments, the combination comprises mCPP, a CB₁ agent, DOB, and AGH-192. In embodiments, the combination comprises mCPP, a CB₁ agent, DOB, and AGH-107. In embodiments, the combination comprises mCPP, a CB₁ agent, DOB, and AMT. In embodiments, the combination comprises mCPP, a CB₁ agent, DOB, and aripiprazole. In embodiments, the combination comprises mCPP, a CB₁ agent, DOB, and AS-19. In embodiments, the combination comprises mCPP, a CB₁ agent, DOB, and E-55888. In embodiments, the combination comprises mCPP, a CB₁ agent, DOB, and LSD. In embodiments, the combination comprises mCPP, a CB₁ agent, DOB, and LP-211. In embodiments, the combination comprises mCPP, a CB₁ agent, DOB, and LP-44.

In embodiments, the combination comprises mCPP, a CB₁ agent, DOET, and 5-CT. In embodiments, the combination comprises mCPP, a CB₁ agent, DOET, and 5-MeO-DALT. In embodiments, the combination comprises mCPP, a CB₁ agent, DOET, and 5-MeO-DMT In embodiments, the combination comprises mCPP, a CB₁ agent, DOET, and 5-MeO-MiPT. In embodiments, the combination comprises mCPP, a CB₁ agent, DOET, and AGH-192. In embodiments, the combination comprises mCPP, a CB₁ agent, DOET, and AGH-107. In embodiments, the combination comprises mCPP, a CB₁ agent, DOET, and AMT. In embodiments, the combination comprises mCPP, a CB₁ agent, DOET, and aripiprazole. In embodiments, the combination comprises mCPP, a CB₁ agent, DOET, and AS-19. In embodiments, the combination comprises mCPP, a CB₁ agent, DOET, and E-55888. In embodiments, the combination comprises mCPP, a CB₁ agent, DOET, and LSD. In embodiments, the combination comprises mCPP, a CB₁ agent, DOET, and LP-211. In embodiments, the combination comprises mCPP, a CB₁ agent, DOET, and LP-44.

In embodiments, the combination comprises mCPP, a CB₁ agent, DOHx, and 5-CT. In embodiments, the combination comprises mCPP, a CB₁ agent, DOHx, and 5-MeO-DALT. In embodiments, the combination comprises mCPP, a CB₁ agent, DOHx, and 5-MeO-DMT In embodiments, the combination comprises mCPP, a CB₁ agent, DOHx, and 5-MeO-MiPT. In embodiments, the combination comprises mCPP, a CB₁ agent, DOHx, and AGH-192. In embodiments, the combination comprises mCPP, a CB₁ agent, DOHx, and AGH-107. In embodiments, the combination comprises mCPP, a CB₁ agent, DOHx, and AMT. In embodiments, the combination comprises mCPP, a CB₁ agent, DOHx, and aripiprazole. In embodiments, the combination comprises mCPP, a CB₁ agent, DOHx, and AS-19. In embodiments, the combination comprises mCPP, a CB₁ agent, DOHx, and E-55888. In embodiments, the combination comprises mCPP, a CB₁ agent, DOHx, and LSD. In embodiments, the combination comprises mCPP, a CB₁ agent, DOHx, and LP-211. In embodiments, the combination comprises mCPP, a CB₁ agent, DOHx, and LP-44.

In embodiments, the combination comprises mCPP, a CB₁ agent, MEM, and 5-CT. In embodiments, the combination comprises mCPP, a CB₁ agent, MEM, and 5-MeO-DALT. In embodiments, the combination comprises mCPP, a CB₁ agent, MEM, and 5-MeO-DMT. In embodiments, the combination comprises mCPP, a CB₁ agent, MEM, and 5-MeO-MiPT In embodiments, the combination comprises mCPP, a CB₁ agent, MEM, and AGH-192. In embodiments, the combination comprises mCPP, a CB₁ agent, MEM, and AGH-107. In embodiments, the combination comprises mCPP, a CB₁ agent, MEM, and AMT. In embodiments, the combination comprises mCPP, a CB₁ agent, MEM, and aripiprazole. In embodiments, the combination comprises mCPP, a CB₁ agent, MEM, and AS-19. In embodiments, the combination comprises mCPP, a CB₁ agent, MEM, and E-55888. In embodiments, the combination comprises mCPP, a CB₁ agent, MEM, and LSD. In embodiments, the combination comprises mCPP, a CB₁ agent, MEM, and LP-211. In embodiments, the combination comprises mCPP, a CB₁ agent, MEM, and LP-44.

In embodiments, the combination comprises mCPP, a CB₁ agent, CPP, and 5-CT. In embodiments, the combination comprises mCPP, a CB₁ agent, CPP, and 5-MeO-DALT. In embodiments, the combination comprises mCPP, a CB₁ agent, CPP, and 5-MeO-DMT In embodiments, the combination comprises mCPP, a CB₁ agent, CPP, and 5-MeO-MiPT In embodiments, the combination comprises mCPP, a CB₁ agent, CPP, and AGH-192. In embodiments, the combination comprises mCPP, a CB₁ agent, CPP, and AGH-107. In embodiments, the combination comprises mCPP, a CB₁ agent, CPP, and AMT. In embodiments, the combination comprises mCPP, a CB₁ agent, CPP, and aripiprazole. In embodiments, the combination comprises mCPP, a CB₁ agent, CPP, and AS-19. In embodiments, the combination comprises mCPP, a CB₁ agent, CPP, and E-55888. In embodiments, the combination comprises mCPP, a CB₁ agent, CPP, and LSD. In embodiments, the combination comprises mCPP, a CB₁ agent, CPP, and LP-211. In embodiments, the combination comprises mCPP, a CB₁ agent, CPP, and LP-44.

In embodiments, the combination comprises mCPP, a CB₁ agent, NBOH-2C-CN, and 5-CT. In embodiments, the combination comprises mCPP, a CB₁ agent, NBOH-2C-CN, and 5-MeO-DALT. In embodiments, the combination comprises mCPP, a CB₁ agent, NBOH-2C-CN, and 5-MeO-DMT. In embodiments, the combination comprises mCPP, a CB₁ agent, NBOH-2C-CN, and 5-MeO-MiPT. In embodiments, the combination comprises mCPP, a CB₁ agent, NBOH-2C-CN, and AGH-192. In embodiments, the combination comprises mCPP, a CB₁ agent, NBOH-2C-CN, and AGH-107. In embodiments, the combination comprises mCPP, a CB₁ agent, NBOH-2C-CN, and AMT. In embodiments, the combination comprises mCPP, a CB₁ agent, NBOH-2C-CN, and aripiprazole. In embodiments, the combination comprises mCPP, a CB₁ agent, NBOH-2C-CN, and AS-19. In embodiments, the combination comprises mCPP, a CB₁ agent, NBOH-2C-CN, and E-55888. In embodiments, the combination comprises mCPP, a CB₁ agent, NBOH-2C-CN, and LSD. In embodiments, the combination comprises mCPP, a CB₁ agent, NBOH-2C-CN, and LP-211. In embodiments, the combination comprises mCPP, a CB₁ agent, NBOH-2C-CN, and LP-44.

In embodiments, the combination comprises mCPP, a CB₁ agent, TCB-2, and 5-CT. In embodiments, the combination comprises mCPP, a CB₁ agent, TCB-2, and 5-MeO-DALT. In embodiments, the combination comprises mCPP, a CB₁ agent, TCB-2, and 5-MeO-DMT. In embodiments, the combination comprises mCPP, a CB₁ agent, TCB-2, and 5-MeO-MiPT. In embodiments, the combination comprises mCPP, a CB₁ agent, TCB-2, and AGH-192. In embodiments, the combination comprises mCPP, a CB₁ agent, TCB-2, and AGH-107. In embodiments, the combination comprises mCPP, a CB₁ agent, TCB-2, and AMT. In embodiments, the combination comprises mCPP, a CB₁ agent, TCB-2, and aripiprazole. In embodiments, the combination comprises mCPP, a CB₁ agent, TCB-2, and AS-19. In embodiments, the combination comprises mCPP, a CB₁ agent, TCB-2, and E-55888. In embodiments, the combination comprises mCPP, a CB₁ agent, TCB-2, and LSD. In embodiments, the combination comprises mCPP, a CB₁ agent, TCB-2, and LP-211. In embodiments, the combination comprises mCPP, a CB₁ agent, TCB-2, and LP-44.

In embodiments, the combination comprises mCPP, a CB₁ agent, WAY 161503, and 5-CT. In embodiments, the combination comprises mCPP, a CB₁ agent, WAY 161503, and 5-MeO-DALT. In embodiments, the combination comprises mCPP, a CB₁ agent, WAY 161503, and 5-MeO-DMT In embodiments, the combination comprises mCPP, a CB₁ agent, WAY 161503, and 5-MeO-MiPT In embodiments, the combination comprises mCPP, a CB₁ agent, WAY 161503, and AGH-192. In embodiments, the combination comprises mCPP, a CB₁ agent, WAY 161503, and AGH-107. In embodiments, the combination comprises mCPP, a CB₁ agent, WAY 161503, and AMT. In embodiments, the combination comprises mCPP, a CB₁ agent, WAY 161503, and aripiprazole. In embodiments, the combination comprises mCPP, a CB₁ agent, WAY 161503, and AS-19. In embodiments, the combination comprises mCPP, a CB₁ agent, WAY 161503, and E-55888. In embodiments, the combination comprises mCPP, a CB₁ agent, WAY 161503, and LSD. In embodiments, the combination comprises mCPP, a CB₁ agent, WAY 161503, and LP-211. In embodiments, the combination comprises mCPP, a CB₁ agent, WAY 161503, and LP-44.

In embodiments, the combination comprises MDMA, a CB₁ agent, 1-methylpsilocin, and 5-CT. In embodiments, the combination comprises MDMA, a CB₁ agent, 1-methylpsilocin, and 5-MeO-DALT. In embodiments, the combination comprises MDMA, a CB₁ agent, 1-methylpsilocin, and 5-MeO-DMT. In embodiments, the combination comprises MDMA, a CB₁ agent, 1-methylpsilocin, and 5-MeO-MiPT. In embodiments, the combination comprises MDMA, a CB₁ agent, 1-methylpsilocin, and AGH-192. In embodiments, the combination comprises MDMA, a CB₁ agent, 1-methylpsilocin, and AGH-107. In embodiments, the combination comprises MDMA, a CB₁ agent, 1-methylpsilocin, and AMT. In embodiments, the combination comprises MDMA, a CB₁ agent, 1-methylpsilocin, and aripiprazole. In embodiments, the combination comprises MDMA, a CB₁ agent, 1-methylpsilocin, and AS-19. In embodiments, the combination comprises MDMA, a CB₁ agent, 1-methylpsilocin, and E-55888. In embodiments, the combination comprises MDMA, a CB₁ agent, 1-methylpsilocin, and LSD. In embodiments, the combination comprises MDMA, a CB₁ agent, 1-methylpsilocin, and LP-211. In embodiments, the combination comprises MDMA, a CB₁ agent, 1-methyl psilocin, and LP-44.

In embodiments, the combination comprises MDMA, a CB₁ agent, 2C-B-FLY, and 5-CT. In embodiments, the combination comprises MDMA, a CB₁ agent, 2C-B-FLY, and 5-MeO-DALT. In embodiments, the combination comprises MDMA, a CB₁ agent, 2C-B-FLY, and 5-MeO-DMT In embodiments, the combination comprises MDMA, a CB₁ agent, 2C-B-FLY, and 5-MeO-MiPT. In embodiments, the combination comprises MDMA, a CB₁ agent, 2C-B-FLY, and AGH-192. In embodiments, the combination comprises MDMA, a CB₁ agent, 2C-B-FLY, and AGH-107. In embodiments, the combination comprises MDMA, a CB₁ agent, 2C-B-FLY, and AMT. In embodiments, the combination comprises MDMA, a CB₁ agent, 2C-B-FLY, and aripiprazole. In embodiments, the combination comprises MDMA, a CB₁ agent, 2C-B-FLY, and AS-19. In embodiments, the combination comprises MDMA, a CB₁ agent, 2C-B-FLY, and E-55888. In embodiments, the combination comprises MDMA, a CB₁ agent, 2C-B-FLY, and LSD. In embodiments, the combination comprises MDMA, a CB₁ agent, 2C-B-FLY, and LP-211. In embodiments, the combination comprises MDMA, a CB₁ agent, 2C-B-FLY, and LP-44.

In embodiments, the combination comprises MDMA, a CB₁ agent, 5-MeO-AMT, and 5-CT. In embodiments, the combination comprises MDMA, a CB₁ agent, 5-MeO-AMT, and 5-MeO-DALT. In embodiments, the combination comprises MDMA, a CB₁ agent, 5-MeO-AMT, and 5-MeO-DMT. In embodiments, the combination comprises MDMA, a CB₁ agent, 5-MeO-AMT, and 5-MeO-MiPT In embodiments, the combination comprises MDMA, a CB₁ agent, 5-MeO-AMT, and AGH-192. In embodiments, the combination comprises MDMA, a CB₁ agent, 5-MeO-AMT, and AGH-107. In embodiments, the combination comprises MDMA, a CB₁ agent, 5-MeO-AMT, and AMT. In embodiments, the combination comprises MDMA, a CB₁ agent, 5-MeO-AMT, and aripiprazole. In embodiments, the combination comprises MDMA, a CB₁ agent, 5-MeO-AMT, and AS-19. In embodiments, the combination comprises MDMA, a CB₁ agent, 5-MeO-AMT, and E-55888. In embodiments, the combination comprises MDMA, a CB₁ agent, 5-MeO-AMT, and LSD. In embodiments, the combination comprises MDMA, a CB₁ agent, 5-MeO-AMT, and LP-211. In embodiments, the combination comprises MDMA, a CB₁ agent, 5-MeO-AMT, and LP-44.

In embodiments, the combination comprises MDMA, a CB₁ agent, AL-37350A, and 5-CT. In embodiments, the combination comprises MDMA, a CB₁ agent, AL-37350A, and 5-MeO-DALT. In embodiments, the combination comprises MDMA, a CB₁ agent, AL-37350A, and 5-MeO-DMT In embodiments, the combination comprises MDMA, a CB₁ agent, AL-37350A, and 5-MeO-MiPT. In embodiments, the combination comprises MDMA, a CB₁ agent, AL-37350A, and AGH-192. In embodiments, the combination comprises MDMA, a CB₁ agent, AL-37350A, and AGH-107. In embodiments, the combination comprises MDMA, a CB₁ agent, AL-37350A, and AMT. In embodiments, the combination comprises MDMA, a CB₁ agent, AL-37350A, and aripiprazole. In embodiments, the combination comprises MDMA, a CB₁ agent, AL-37350A, and AS-19. In embodiments, the combination comprises MDMA, a CB₁ agent, AL-37350A, and E-55888. In embodiments, the combination comprises MDMA, a CB₁ agent, AL-37350A, and LSD. In embodiments, the combination comprises MDMA, a CB₁ agent, AL-37350A, and LP-211. In embodiments, the combination comprises MDMA, a CB₁ agent, AL-37350A, and LP-44.

In embodiments, the combination comprises MDMA, a CB₁ agent, CP 809101, and 5-CT. In embodiments, the combination comprises MDMA, a CB₁ agent, CP 809101, and 5-MeO-DALT. In embodiments, the combination comprises MDMA, a CB₁ agent, CP 809101, and 5-MeO-DMT In embodiments, the combination comprises MDMA, a CB₁ agent, CP 809101, and 5-MeO-MiPT. In embodiments, the combination comprises MDMA, a CB₁ agent, CP 809101, and AGH-192. In embodiments, the combination comprises MDMA, a CB₁ agent, CP 809101, and AGH-107. In embodiments, the combination comprises MDMA, a CB₁ agent, CP 809101, and AMT. In embodiments, the combination comprises MDMA, a CB₁ agent, CP 809101, and aripiprazole. In embodiments, the combination comprises MDMA, a CB₁ agent, CP 809101, and AS-19. In embodiments, the combination comprises MDMA, a CB₁ agent, CP 809101, and E-55888. In embodiments, the combination comprises MDMA, a CB₁ agent, CP 809101, and LSD. In embodiments, the combination comprises MDMA, a CB₁ agent, CP 809101, and LP-211. In embodiments, the combination comprises MDMA, a CB₁ agent, CP 809101, and LP-44.

In embodiments, the combination comprises MDMA, a CB₁ agent, DALT, and 5-CT. In embodiments, the combination comprises MDMA, a CB₁ agent, DALT, and 5-MeO-DALT. In embodiments, the combination comprises MDMA, a CB₁ agent, DALT, and 5-MeO-DMT. In embodiments, the combination comprises MDMA, a CB₁ agent, DALT, and 5-MeO-MiPT. In embodiments, the combination comprises MDMA, a CB₁ agent, DALT, and AGH-192. In embodiments, the combination comprises MDMA, a CB₁ agent, DALT, and AGH-107. In embodiments, the combination comprises MDMA, a CB₁ agent, DALT, and AMT. In embodiments, the combination comprises MDMA, a CB₁ agent, DALT, and aripiprazole. In embodiments, the combination comprises MDMA, a CB₁ agent, DALT, and AS-19. In embodiments, the combination comprises MDMA, a CB₁ agent, DALT, and E-55888. In embodiments, the combination comprises MDMA, a CB₁ agent, DALT, and LSD. In embodiments, the combination comprises MDMA, a CB₁ agent, DALT, and LP-211. In embodiments, the combination comprises MDMA, a CB₁ agent, DALT, and LP-44.

In embodiments, the combination comprises MDMA, a CB₁ agent, DOB, and 5-CT. In embodiments, the combination comprises MDMA, a CB₁ agent, DOB, and 5-MeO-DALT. In embodiments, the combination comprises MDMA, a CB₁ agent, DOB, and 5-MeO-DMT. In embodiments, the combination comprises MDMA, a CB₁ agent, DOB, and 5-MeO-MiPT In embodiments, the combination comprises MDMA, a CB₁ agent, DOB, and AGH-192. In embodiments, the combination comprises MDMA, a CB₁ agent, DOB, and AGH-107. In embodiments, the combination comprises MDMA, a CB₁ agent, DOB, and AMT. In embodiments, the combination comprises MDMA, a CB₁ agent, DOB, and aripiprazole. In embodiments, the combination comprises MDMA, a CB₁ agent, DOB, and AS-19. In embodiments, the combination comprises MDMA, a CB₁ agent, DOB, and E-55888. In embodiments, the combination comprises MDMA, a CB₁ agent, DOB, and LSD. In embodiments, the combination comprises MDMA, a CB₁ agent, DOB, and LP-211. In embodiments, the combination comprises MDMA, a CB₁ agent, DOB, and LP-44.

In embodiments, the combination comprises MDMA, a CB₁ agent, DOET, and 5-CT. In embodiments, the combination comprises MDMA, a CB₁ agent, DOET, and 5-MeO-DALT. In embodiments, the combination comprises MDMA, a CB₁ agent, DOET, and 5-MeO-DMT. In embodiments, the combination comprises MDMA, a CB₁ agent, DOET, and 5-MeO-MiPT. In embodiments, the combination comprises MDMA, a CB₁ agent, DOET, and AGH-192. In embodiments, the combination comprises MDMA, a CB₁ agent, DOET, and AGH-107. In embodiments, the combination comprises MDMA, a CB₁ agent, DOET, and AMT. In embodiments, the combination comprises MDMA, a CB₁ agent, DOET, and aripiprazole. In embodiments, the combination comprises MDMA, a CB₁ agent, DOET, and AS-19. In embodiments, the combination comprises MDMA, a CB₁ agent, DOET, and E-55888. In embodiments, the combination comprises MDMA, a CB₁ agent, DOET, and LSD. In embodiments, the combination comprises MDMA, a CB₁ agent, DOET, and LP-211. In embodiments, the combination comprises MDMA, a CB₁ agent, DOET, and LP-44.

In embodiments, the combination comprises MDMA, a CB₁ agent, DOHx, and 5-CT. In embodiments, the combination comprises MDMA, a CB₁ agent, DOHx, and 5-MeO-DALT. In embodiments, the combination comprises MDMA, a CB₁ agent, DOHx, and 5-MeO-DMT. In embodiments, the combination comprises MDMA, a CB₁ agent, DOHx, and 5-MeO-MiPT. In embodiments, the combination comprises MDMA, a CB₁ agent, DOHx, and AGH-192. In embodiments, the combination comprises MDMA, a CB₁ agent, DOHx, and AGH-107. In embodiments, the combination comprises MDMA, a CB₁ agent, DOHx, and AMT. In embodiments, the combination comprises MDMA, a CB₁ agent, DOHx, and aripiprazole. In embodiments, the combination comprises MDMA, a CB₁ agent, DOHx, and AS-19. In embodiments, the combination comprises MDMA, a CB₁ agent, DOHx, and E-55888. In embodiments, the combination comprises MDMA, a CB₁ agent, DOHx, and LSD. In embodiments, the combination comprises MDMA, a CB₁ agent, DOHx, and LP-211. In embodiments, the combination comprises MDMA, a CB₁ agent, DOHx, and LP-44.

In embodiments, the combination comprises MDMA, a CB₁ agent, MEM, and 5-CT. In embodiments, the combination comprises MDMA, a CB₁ agent, MEM, and 5-MeO-DALT. In embodiments, the combination comprises MDMA, a CB₁ agent, MEM, and 5-MeO-DMT In embodiments, the combination comprises MDMA, a CB₁ agent, MEM, and 5-MeO-MiPT In embodiments, the combination comprises MDMA, a CB₁ agent, MEM, and AGH-192. In embodiments, the combination comprises MDMA, a CB₁ agent, MEM, and AGH-107. In embodiments, the combination comprises MDMA, a CB₁ agent, MEM, and AMT. In embodiments, the combination comprises MDMA, a CB₁ agent, MEM, and aripiprazole. In embodiments, the combination comprises MDMA, a CB₁ agent, MEM, and AS-19. In embodiments, the combination comprises MDMA, a CB₁ agent, MEM, and E-55888. In embodiments, the combination comprises MDMA, a CB₁ agent, MEM, and LSD. In embodiments, the combination comprises MDMA, a CB₁ agent, MEM, and LP-211. In embodiments, the combination comprises MDMA, a CB₁ agent, MEM, and LP-44.

In embodiments, the combination comprises MDMA, a CB₁ agent, CPP, and 5-CT. In embodiments, the combination comprises MDMA, a CB₁ agent, CPP, and 5-MeO-DALT. In embodiments, the combination comprises MDMA, a CB₁ agent, CPP, and 5-MeO-DMT In embodiments, the combination comprises MDMA, a CB₁ agent, CPP, and 5-MeO-MiPT. In embodiments, the combination comprises MDMA, a CB₁ agent, CPP, and AGH-192. In embodiments, the combination comprises MDMA, a CB₁ agent, CPP, and AGH-107. In embodiments, the combination comprises MDMA, a CB₁ agent, CPP, and AMT. In embodiments, the combination comprises MDMA, a CB₁ agent, CPP, and aripiprazole. In embodiments, the combination comprises MDMA, a CB₁ agent, CPP, and AS-19. In embodiments, the combination comprises MDMA, a CB₁ agent, CPP, and E-55888. In embodiments, the combination comprises MDMA, a CB₁ agent, CPP, and LSD. In embodiments, the combination comprises MDMA, a CB₁ agent, CPP, and LP-211. In embodiments, the combination comprises MDMA, a CB₁ agent, CPP, and LP-44.

In embodiments, the combination comprises MDMA, a CB₁ agent, NBOH-2C-CN, and 5-CT. In embodiments, the combination comprises MDMA, a CB₁ agent, NBOH-2C-CN, and 5-MeO-DALT. In embodiments, the combination comprises MDMA, a CB₁ agent, NBOH-2C-CN, and 5-MeO-DMT. In embodiments, the combination comprises MDMA, a CB₁ agent, NBOH-2C-CN, and 5-MeO-MiPT In embodiments, the combination comprises MDMA, a CB₁ agent, NBOH-2C-CN, and AGH-192. In embodiments, the combination comprises MDMA, a CB₁ agent, NBOH-2C-CN, and AGH-107. In embodiments, the combination comprises MDMA, a CB₁ agent, NBOH-2C-CN, and AMT. In embodiments, the combination comprises MDMA, a CB₁ agent, NBOH-2C-CN, and aripiprazole. In embodiments, the combination comprises MDMA, a CB₁ agent, NBOH-2C-CN, and AS-19. In embodiments, the combination comprises MDMA, a CB₁ agent, NBOH-2C-CN, and E-55888. In embodiments, the combination comprises MDMA, a CB₁ agent, NBOH-2C-CN, and LSD. In embodiments, the combination comprises MDMA, a CB₁ agent, NBOH-2C-CN, and LP-211. In embodiments, the combination comprises MDMA, a CB₁ agent, NBOH-2C-CN, and LP-44.

In embodiments, the combination comprises MDMA, a CB₁ agent, TCB-2, and 5-CT. In embodiments, the combination comprises MDMA, a CB₁ agent, TCB-2, and 5-MeO-DALT. In embodiments, the combination comprises MDMA, a CB₁ agent, TCB-2, and 5-MeO-DMT In embodiments, the combination comprises MDMA, a CB₁ agent, TCB-2, and 5-MeO-MiPT. In embodiments, the combination comprises MDMA, a CB₁ agent, TCB-2, and AGH-192. In embodiments, the combination comprises MDMA, a CB₁ agent, TCB-2, and AGH-107. In embodiments, the combination comprises MDMA, a CB₁ agent, TCB-2, and AMT. In embodiments, the combination comprises MDMA, a CB₁ agent, TCB-2, and aripiprazole. In embodiments, the combination comprises MDMA, a CB₁ agent, TCB-2, and AS-19. In embodiments, the combination comprises MDMA, a CB₁ agent, TCB-2, and E-55888. In embodiments, the combination comprises MDMA, a CB₁ agent, TCB-2, and LSD. In embodiments, the combination comprises MDMA, a CB₁ agent, TCB-2, and LP-211. In embodiments, the combination comprises MDMA, a CB₁ agent, TCB-2, and LP-44.

In embodiments, the combination comprises MDMA, a CB₁ agent, WAY 161503, and 5-CT. In embodiments, the combination comprises MDMA, a CB₁ agent, WAY 161503, and 5-MeO-DALT. In embodiments, the combination comprises MDMA, a CB₁ agent, WAY 161503, and 5-MeO-DMT In embodiments, the combination comprises MDMA, a CB₁ agent, WAY 161503, and 5-MeO-MiPT. In embodiments, the combination comprises MDMA, a CB₁ agent, WAY 161503, and AGH-192. In embodiments, the combination comprises MDMA, a CB₁ agent, WAY 161503, and AGH-107. In embodiments, the combination comprises MDMA, a CB₁ agent, WAY 161503, and AMT. In embodiments, the combination comprises MDMA, a CB₁ agent, WAY 161503, and aripiprazole. In embodiments, the combination comprises MDMA, a CB₁ agent, WAY 161503, and AS-19. In embodiments, the combination comprises MDMA, a CB₁ agent, WAY 161503, and E-55888. In embodiments, the combination comprises MDMA, a CB₁ agent, WAY 161503, and LSD. In embodiments, the combination comprises MDMA, a CB₁ agent, WAY 161503, and LP-211. In embodiments, the combination comprises MDMA, a CB₁ agent, WAY 161503, and LP-44.

In embodiments, the combination comprises memantine, a CB₁ agent, 1-methylpsilocin, and 5-CT. In embodiments, the combination comprises memantine, a CB₁ agent, 1-methylpsilocin, and 5-MeO-DALT. In embodiments, the combination comprises memantine, a CB₁ agent, 1-methylpsilocin, and 5-MeO-DMT. In embodiments, the combination comprises memantine, a CB₁ agent, 1-methylpsilocin, and 5-MeO-MiPT In embodiments, the combination comprises memantine, a CB₁ agent, 1-methylpsilocin, and AGH-192. In embodiments, the combination comprises memantine, a CB₁ agent, 1-methylpsilocin, and AGH-107. In embodiments, the combination comprises memantine, a CB₁ agent, 1-methylpsilocin, and AMT. In embodiments, the combination comprises memantine, a CB₁ agent, 1-methylpsilocin, and aripiprazole. In embodiments, the combination comprises memantine, a CB₁ agent, 1-methylpsilocin, and AS-19. In embodiments, the combination comprises memantine, a CB₁ agent, 1-methylpsilocin, and E-55888. In embodiments, the combination comprises memantine, a CB₁ agent, 1-methylpsilocin, and LSD. In embodiments, the combination comprises memantine, a CB₁ agent, 1-methylpsilocin, and LP-211. In embodiments, the combination comprises memantine, a CB₁ agent, 1-methylpsilocin, and LP-44.

In embodiments, the combination comprises memantine, a CB₁ agent, 2C-B-FLY, and 5-CT. In embodiments, the combination comprises memantine, a CB₁ agent, 2C-B-FLY, and 5-MeO-DALT. In embodiments, the combination comprises memantine, a CB₁ agent, 2C-B-FLY, and 5-MeO-DMT In embodiments, the combination comprises memantine, a CB₁ agent, 2C-B-FLY, and 5-MeO-MiPT. In embodiments, the combination comprises memantine, a CB₁ agent, 2C-B-FLY, and AGH-192. In embodiments, the combination comprises memantine, a CB₁ agent, 2C-B-FLY, and AGH-107. In embodiments, the combination comprises memantine, a CB₁ agent, 2C-B-FLY, and AMT. In embodiments, the combination comprises memantine, a CB₁ agent, 2C-B-FLY, and aripiprazole. In embodiments, the combination comprises memantine, a CB₁ agent, 2C-B-FLY, and AS-19. In embodiments, the combination comprises memantine, a CB₁ agent, 2C-B-FLY, and E-55888. In embodiments, the combination comprises memantine, a CB₁ agent, 2C-B-FLY, and LSD. In embodiments, the combination comprises memantine, a CB₁ agent, 2C-B-FLY, and LP-211. In embodiments, the combination comprises memantine, a CB₁ agent, 2C-B-FLY, and LP-44.

In embodiments, the combination comprises memantine, a CB₁ agent, 5-MeO-AMT, and 5-CT. In embodiments, the combination comprises memantine, a CB₁ agent, 5-MeO-AMT, and 5-MeO-DALT. In embodiments, the combination comprises memantine, a CB₁ agent, 5-MeO-AMT, and 5-MeO-DMT In embodiments, the combination comprises memantine, a CB₁ agent, 5-MeO-AMT, and 5-MeO-MiPT. In embodiments, the combination comprises memantine, a CB₁ agent, 5-MeO-AMT, and AGH-192. In embodiments, the combination comprises memantine, a CB₁ agent, 5-MeO-AMT, and AGH-107. In embodiments, the combination comprises memantine, a CB₁ agent, 5-MeO-AMT, and AMT. In embodiments, the combination comprises memantine, a CB₁ agent, 5-MeO-AMT, and aripiprazole. In embodiments, the combination comprises memantine, a CB₁ agent, 5-MeO-AMT, and AS-19. In embodiments, the combination comprises memantine, a CB₁ agent, 5-MeO-AMT, and E-55888. In embodiments, the combination comprises memantine, a CB₁ agent, 5-MeO-AMT, and LSD. In embodiments, the combination comprises memantine, a CB₁ agent, 5-MeO-AMT, and LP-211. In embodiments, the combination comprises memantine, a CB₁ agent, 5-MeO-AMT, and LP-44.

In embodiments, the combination comprises memantine, a CB₁ agent, AL-37350A, and 5-CT. In embodiments, the combination comprises memantine, a CB₁ agent, AL-37350A, and 5-MeO-DALT. In embodiments, the combination comprises memantine, a CB₁ agent, AL-37350A, and 5-MeO-DMT In embodiments, the combination comprises memantine, a CB₁ agent, AL-37350A, and 5-MeO-MiPT. In embodiments, the combination comprises memantine, a CB₁ agent, AL-37350A, and AGH-192. In embodiments, the combination comprises memantine, a CB₁ agent, AL-37350A, and AGH-107. In embodiments, the combination comprises memantine, a CB₁ agent, AL-37350A, and AMT. In embodiments, the combination comprises memantine, a CB₁ agent, AL-37350A, and aripiprazole. In embodiments, the combination comprises memantine, a CB₁ agent, AL-37350A, and AS-19. In embodiments, the combination comprises memantine, a CB₁ agent, AL-37350A, and E-55888. In embodiments, the combination comprises memantine, a CB₁ agent, AL-37350A, and LSD. In embodiments, the combination comprises memantine, a CB₁ agent, AL-37350A, and LP-211. In embodiments, the combination comprises memantine, a CB₁ agent, AL-37350A, and LP-44.

In embodiments, the combination comprises memantine, a CB₁ agent, CP 809101, and 5-CT. In embodiments, the combination comprises memantine, a CB₁ agent, CP 809101, and 5-MeO-DALT. In embodiments, the combination comprises memantine, a CB₁ agent, CP 809101, and 5-MeO-DMT In embodiments, the combination comprises memantine, a CB₁ agent, CP 809101, and 5-MeO-MiPT. In embodiments, the combination comprises memantine, a CB₁ agent, CP 809101, and AGH-192. In embodiments, the combination comprises memantine, a CB₁ agent, CP 809101, and AGH-107. In embodiments, the combination comprises memantine, a CB₁ agent, CP 809101, and AMT. In embodiments, the combination comprises memantine, a CB₁ agent, CP 809101, and aripiprazole. In embodiments, the combination comprises memantine, a CB₁ agent, CP 809101, and AS-19. In embodiments, the combination comprises memantine, a CB₁ agent, CP 809101, and E-55888. In embodiments, the combination comprises memantine, a CB₁ agent, CP 809101, and LSD. In embodiments, the combination comprises memantine, a CB₁ agent, CP 809101, and LP-211. In embodiments, the combination comprises memantine, a CB₁ agent, CP 809101, and LP-44.

In embodiments, the combination comprises memantine, a CB₁ agent, DALT, and 5-CT. In embodiments, the combination comprises memantine, a CB₁ agent, DALT, and 5-MeO-DALT. In embodiments, the combination comprises memantine, a CB₁ agent, DALT, and 5-MeO-DMT. In embodiments, the combination comprises memantine, a CB₁ agent, DALT, and 5-MeO-MiPT. In embodiments, the combination comprises memantine, a CB₁ agent, DALT, and AGH-192. In embodiments, the combination comprises memantine, a CB₁ agent, DALT, and AGH-107. In embodiments, the combination comprises memantine, a CB₁ agent, DALT, and AMT. In embodiments, the combination comprises memantine, a CB₁ agent, DALT, and aripiprazole. In embodiments, the combination comprises memantine, a CB₁ agent, DALT, and AS-19. In embodiments, the combination comprises memantine, a CB₁ agent, DALT, and E-55888. In embodiments, the combination comprises memantine, a CB₁ agent, DALT, and LSD. In embodiments, the combination comprises memantine, a CB₁ agent, DALT, and LP-211. In embodiments, the combination comprises memantine, a CB₁ agent, DALT, and LP-44.

In embodiments, the combination comprises memantine, a CB₁ agent, DOB, and 5-CT. In embodiments, the combination comprises memantine, a CB₁ agent, DOB, and 5-MeO-DALT. In embodiments, the combination comprises memantine, a CB₁ agent, DOB, and 5-MeO-DMT. In embodiments, the combination comprises memantine, a CB₁ agent, DOB, and 5-MeO-MiPT. In embodiments, the combination comprises memantine, a CB₁ agent, DOB, and AGH-192. In embodiments, the combination comprises memantine, a CB₁ agent, DOB, and AGH-107. In embodiments, the combination comprises memantine, a CB₁ agent, DOB, and AMT. In embodiments, the combination comprises memantine, a CB₁ agent, DOB, and aripiprazole. In embodiments, the combination comprises memantine, a CB₁ agent, DOB, and AS-19. In embodiments, the combination comprises memantine, a CB₁ agent, DOB, and E-55888. In embodiments, the combination comprises memantine, a CB₁ agent, DOB, and LSD. In embodiments, the combination comprises memantine, a CB₁ agent, DOB, and LP-211. In embodiments, the combination comprises memantine, a CB₁ agent, DOB, and LP-44.

In embodiments, the combination comprises memantine, a CB₁ agent, DOET, and 5-CT. In embodiments, the combination comprises memantine, a CB₁ agent, DOET, and 5-MeO-DALT. In embodiments, the combination comprises memantine, a CB₁ agent, DOET, and 5-MeO-DMT. In embodiments, the combination comprises memantine, a CB₁ agent, DOET, and 5-MeO-MiPT In embodiments, the combination comprises memantine, a CB₁ agent, DOET, and AGH-192. In embodiments, the combination comprises memantine, a CB₁ agent, DOET, and AGH-107. In embodiments, the combination comprises memantine, a CB₁ agent, DOET, and AMT. In embodiments, the combination comprises memantine, a CB₁ agent, DOET, and aripiprazole. In embodiments, the combination comprises memantine, a CB₁ agent, DOET, and AS-19. In embodiments, the combination comprises memantine, a CB₁ agent, DOET, and E-55888. In embodiments, the combination comprises memantine, a CB₁ agent, DOET, and LSD. In embodiments, the combination comprises memantine, a CB₁ agent, DOET, and LP-211. In embodiments, the combination comprises memantine, a CB₁ agent, DOET, and LP-44.

In embodiments, the combination comprises memantine, a CB₁ agent, DOHx, and 5-CT. In embodiments, the combination comprises memantine, a CB₁ agent, DOHx, and 5-MeO-DALT. In embodiments, the combination comprises memantine, a CB₁ agent, DOHx, and 5-MeO-DMT. In embodiments, the combination comprises memantine, a CB₁ agent, DOHx, and 5-MeO-MiPT In embodiments, the combination comprises memantine, a CB₁ agent, DOHx, and AGH-192. In embodiments, the combination comprises memantine, a CB₁ agent, DOHx, and AGH-107. In embodiments, the combination comprises memantine, a CB₁ agent, DOHx, and AMT. In embodiments, the combination comprises memantine, a CB₁ agent, DOHx, and aripiprazole. In embodiments, the combination comprises memantine, a CB₁ agent, DOHx, and AS-19. In embodiments, the combination comprises memantine, a CB₁ agent, DOHx, and E-55888. In embodiments, the combination comprises memantine, a CB₁ agent, DOHx, and LSD. In embodiments, the combination comprises memantine, a CB₁ agent, DOHx, and LP-211. In embodiments, the combination comprises memantine, a CB₁ agent, DOHx, and LP-44.

In embodiments, the combination comprises memantine, a CB₁ agent, MEM, and 5-CT. In embodiments, the combination comprises memantine, a CB₁ agent, MEM, and 5-MeO-DALT. In embodiments, the combination comprises memantine, a CB₁ agent, MEM, and 5-MeO-DMT. In embodiments, the combination comprises memantine, a CB₁ agent, MEM, and 5-MeO-MiPT In embodiments, the combination comprises memantine, a CB₁ agent, MEM, and AGH-192. In embodiments, the combination comprises memantine, a CB₁ agent, MEM, and AGH-107. In embodiments, the combination comprises memantine, a CB₁ agent, MEM, and AMT. In embodiments, the combination comprises memantine, a CB₁ agent, MEM, and aripiprazole. In embodiments, the combination comprises memantine, a CB₁ agent, MEM, and AS-19. In embodiments, the combination comprises memantine, a CB₁ agent, MEM, and E-55888. In embodiments, the combination comprises memantine, a CB₁ agent, MEM, and LSD. In embodiments, the combination comprises memantine, a CB₁ agent, MEM, and LP-211. In embodiments, the combination comprises memantine, a CB₁ agent, MEM, and LP-44.

In embodiments, the combination comprises memantine, a CB₁ agent, CPP, and 5-CT. In embodiments, the combination comprises memantine, a CB₁ agent, CPP, and 5-MeO-DALT. In embodiments, the combination comprises memantine, a CB₁ agent, CPP, and 5-MeO-DMT. In embodiments, the combination comprises memantine, a CB₁ agent, CPP, and 5-MeO-MiPT In embodiments, the combination comprises memantine, a CB₁ agent, CPP, and AGH-192. In embodiments, the combination comprises memantine, a CB₁ agent, CPP, and AGH-107. In embodiments, the combination comprises memantine, a CB₁ agent, CPP, and AMT. In embodiments, the combination comprises memantine, a CB₁ agent, CPP, and aripiprazole. In embodiments, the combination comprises memantine, a CB₁ agent, CPP, and AS-19. In embodiments, the combination comprises memantine, a CB₁ agent, CPP, and E-55888. In embodiments, the combination comprises memantine, a CB₁ agent, CPP, and LSD. In embodiments, the combination comprises memantine, a CB₁ agent, CPP, and LP-211. In embodiments, the combination comprises memantine, a CB₁ agent, CPP, and LP-44.

In embodiments, the combination comprises memantine, a CB₁ agent, NBOH-2C-CN, and 5-CT. In embodiments, the combination comprises memantine, a CB₁ agent, NBOH-2C-CN, and 5-MeO-DALT. In embodiments, the combination comprises memantine, a CB₁ agent, NBOH-2C-CN, and 5-MeO-DMT In embodiments, the combination comprises memantine, a CB₁ agent, NBOH-2C-CN, and 5-MeO-MiPT. In embodiments, the combination comprises memantine, a CB₁ agent, NBOH-2C-CN, and AGH-192. In embodiments, the combination comprises memantine, a CB₁ agent, NBOH-2C-CN, and AGH-107. In embodiments, the combination comprises memantine, a CB₁ agent, NBOH-2C-CN, and AMT. In embodiments, the combination comprises memantine, a CB₁ agent, NBOH-2C-CN, and aripiprazole. In embodiments, the combination comprises memantine, a CB₁ agent, NBOH-2C-CN, and AS-19. In embodiments, the combination comprises memantine, a CB₁ agent, NBOH-2C-CN, and E-55888. In embodiments, the combination comprises memantine, a CB₁ agent, NBOH-2C-CN, and LSD. In embodiments, the combination comprises memantine, a CB₁ agent, NBOH-2C-CN, and LP-211. In embodiments, the combination comprises memantine, a CB₁ agent, NBOH-2C-CN, and LP-44.

In embodiments, the combination comprises memantine, a CB₁ agent, TCB-2, and 5-CT. In embodiments, the combination comprises memantine, a CB₁ agent, TCB-2, and 5-MeO-DALT. In embodiments, the combination comprises memantine, a CB₁ agent, TCB-2, and 5-MeO-DMT In embodiments, the combination comprises memantine, a CB₁ agent, TCB-2, and 5-MeO-MiPT In embodiments, the combination comprises memantine, a CB₁ agent, TCB-2, and AGH-192. In embodiments, the combination comprises memantine, a CB₁ agent, TCB-2, and AGH-107. In embodiments, the combination comprises memantine, a CB₁ agent, TCB-2, and AMT. In embodiments, the combination comprises memantine, a CB₁ agent, TCB-2, and aripiprazole. In embodiments, the combination comprises memantine, a CB₁ agent, TCB-2, and AS-19. In embodiments, the combination comprises memantine, a CB₁ agent, TCB-2, and E-55888. In embodiments, the combination comprises memantine, a CB₁ agent, TCB-2, and LSD. In embodiments, the combination comprises memantine, a CB₁ agent, TCB-2, and LP-211. In embodiments, the combination comprises memantine, a CB₁ agent, TCB-2, and LP-44.

In embodiments, the combination comprises memantine, a CB₁ agent, WAY 161503, and 5-CT. In embodiments, the combination comprises memantine, a CB₁ agent, WAY 161503, and 5-MeO-DALT In embodiments, the combination comprises memantine, a CB₁ agent, WAY 161503, and 5-MeO-DMT. In embodiments, the combination comprises memantine, a CB₁ agent, WAY 161503, and 5-MeO-MiPT In embodiments, the combination comprises memantine, a CB₁ agent, WAY 161503, and AGH-192. In embodiments, the combination comprises memantine, a CB₁ agent, WAY 161503, and AGH-107. In embodiments, the combination comprises memantine, a CB₁ agent, WAY 161503, and AMT. In embodiments, the combination comprises memantine, a CB₁ agent, WAY 161503, and aripiprazole. In embodiments, the combination comprises memantine, a CB₁ agent, WAY 161503, and AS-19. In embodiments, the combination comprises memantine, a CB₁ agent, WAY 161503, and E-55888. In embodiments, the combination comprises memantine, a CB₁ agent, WAY 161503, and LSD. In embodiments, the combination comprises memantine, a CB₁ agent, WAY 161503, and LP-211. In embodiments, the combination comprises memantine, a CB₁ agent, WAY 161503, and LP-44.

In embodiments, the combination comprises moxonidine, a CB₁ agent, 1-methylpsilocin, and 5-CT. In embodiments, the combination comprises moxonidine, a CB₁ agent, 1-methylpsilocin, and 5-MeO-DALT. In embodiments, the combination comprises moxonidine, a CB₁ agent, 1-methylpsilocin, and 5-MeO-DMT. In embodiments, the combination comprises moxonidine, a CB₁ agent, 1-methylpsilocin, and 5-MeO-MiPT. In embodiments, the combination comprises moxonidine, a CB₁ agent, 1-methylpsilocin, and AGH-192. In embodiments, the combination comprises moxonidine, a CB₁ agent, 1-methylpsilocin, and AGH-107. In embodiments, the combination comprises moxonidine, a CB₁ agent, 1-methylpsilocin, and AMT. In embodiments, the combination comprises moxonidine, a CB₁ agent, 1-methylpsilocin, and aripiprazole. In embodiments, the combination comprises moxonidine, a CB₁ agent, 1-methylpsilocin, and AS-19. In embodiments, the combination comprises moxonidine, a CB₁ agent, 1-methylpsilocin, and E-55888. In embodiments, the combination comprises moxonidine, a CB₁ agent, 1-methylpsilocin, and LSD. In embodiments, the combination comprises moxonidine, a CB₁ agent, 1-methylpsilocin, and LP-211. In embodiments, the combination comprises moxonidine, a CB₁ agent, 1-methyl psilocin, and LP-44.

In embodiments, the combination comprises moxonidine, a CB₁ agent, 2C-B-FLY, and 5-CT. In embodiments, the combination comprises moxonidine, a CB₁ agent, 2C-B-FLY, and 5-MeO-DALT. In embodiments, the combination comprises moxonidine, a CB₁ agent, 2C-B-FLY, and 5-MeO-DMT. In embodiments, the combination comprises moxonidine, a CB₁ agent, 2C-B-FLY, and 5-MeO-MiPT In embodiments, the combination comprises moxonidine, a CB₁ agent, 2C-B-FLY, and AGH-192. In embodiments, the combination comprises moxonidine, a CB₁ agent, 2C-B-FLY, and AGH-107. In embodiments, the combination comprises moxonidine, a CB₁ agent, 2C-B-FLY, and AMT. In embodiments, the combination comprises moxonidine, a CB₁ agent, 2C-B-FLY, and aripiprazole. In embodiments, the combination comprises moxonidine, a CB₁ agent, 2C-B-FLY, and AS-19. In embodiments, the combination comprises moxonidine, a CB₁ agent, 2C-B-FLY, and E-55888. In embodiments, the combination comprises moxonidine, a CB₁ agent, 2C-B-FLY, and LSD. In embodiments, the combination comprises moxonidine, a CB₁ agent, 2C-B-FLY, and LP-211. In embodiments, the combination comprises moxonidine, a CB₁ agent, 2C-B-FLY, and LP-44.

In embodiments, the combination comprises moxonidine, a CB₁ agent, 5-MeO-AMT, and 5-CT. In embodiments, the combination comprises moxonidine, a CB₁ agent, 5-MeO-AMT, and 5-MeO-DALT. In embodiments, the combination comprises moxonidine, a CB₁ agent, 5-MeO-AMT, and 5-MeO-DMT. In embodiments, the combination comprises moxonidine, a CB₁ agent, 5-MeO-AMT, and 5-MeO-MiPT In embodiments, the combination comprises moxonidine, a CB₁ agent, 5-MeO-AMT, and AGH-192. In embodiments, the combination comprises moxonidine, a CB₁ agent, 5-MeO-AMT, and AGH-107. In embodiments, the combination comprises moxonidine, a CB₁ agent, 5-MeO-AMT, and AMT. In embodiments, the combination comprises moxonidine, a CB₁ agent, 5-MeO-AMT, and aripiprazole. In embodiments, the combination comprises moxonidine, a CB₁ agent, 5-MeO-AMT, and AS-19. In embodiments, the combination comprises moxonidine, a CB₁ agent, 5-MeO-AMT, and E-55888. In embodiments, the combination comprises moxonidine, a CB₁ agent, 5-MeO-AMT, and LSD. In embodiments, the combination comprises moxonidine, a CB₁ agent, 5-MeO-AMT, and LP-211. In embodiments, the combination comprises moxonidine, a CB₁ agent, 5-MeO-AMT, and LP-44.

In embodiments, the combination comprises moxonidine, a CB₁ agent, AL-37350A, and 5-CT. In embodiments, the combination comprises moxonidine, a CB₁ agent, AL-37350A, and 5-MeO-DALT. In embodiments, the combination comprises moxonidine, a CB₁ agent, AL-37350A, and 5-MeO-DMT. In embodiments, the combination comprises moxonidine, a CB₁ agent, AL-37350A, and 5-MeO-MiPT In embodiments, the combination comprises moxonidine, a CB₁ agent, AL-37350A, and AGH-192. In embodiments, the combination comprises moxonidine, a CB₁ agent, AL-37350A, and AGH-107. In embodiments, the combination comprises moxonidine, a CB₁ agent, AL-37350A, and AMT. In embodiments, the combination comprises moxonidine, a CB₁ agent, AL-37350A, and aripiprazole. In embodiments, the combination comprises moxonidine, a CB₁ agent, AL-37350A, and AS-19. In embodiments, the combination comprises moxonidine, a CB₁ agent, AL-37350A, and E-55888. In embodiments, the combination comprises moxonidine, a CB₁ agent, AL-37350A, and LSD. In embodiments, the combination comprises moxonidine, a CB₁ agent, AL-37350A, and LP-211. In embodiments, the combination comprises moxonidine, a CB₁ agent, AL-37350A, and LP-44.

In embodiments, the combination comprises moxonidine, a CB₁ agent, CP 809101, and 5-CT. In embodiments, the combination comprises moxonidine, a CB₁ agent, CP 809101, and 5-MeO-DALT. In embodiments, the combination comprises moxonidine, a CB₁ agent, CP 809101, and 5-MeO-DMT. In embodiments, the combination comprises moxonidine, a CB₁ agent, CP 809101, and 5-MeO-MiPT In embodiments, the combination comprises moxonidine, a CB₁ agent, CP 809101, and AGH-192. In embodiments, the combination comprises moxonidine, a CB₁ agent, CP 809101, and AGH-107. In embodiments, the combination comprises moxonidine, a CB₁ agent, CP 809101, and AMT. In embodiments, the combination comprises moxonidine, a CB₁ agent, CP 809101, and aripiprazole. In embodiments, the combination comprises moxonidine, a CB₁ agent, CP 809101, and AS-19. In embodiments, the combination comprises moxonidine, a CB₁ agent, CP 809101, and E-55888. In embodiments, the combination comprises moxonidine, a CB₁ agent, CP 809101, and LSD. In embodiments, the combination comprises moxonidine, a CB₁ agent, CP 809101, and LP-211. In embodiments, the combination comprises moxonidine, a CB₁ agent, CP 809101, and LP-44.

In embodiments, the combination comprises moxonidine, a CB₁ agent, DALT, and 5-CT. In embodiments, the combination comprises moxonidine, a CB₁ agent, DALT, and 5-MeO-DALT. In embodiments, the combination comprises moxonidine, a CB₁ agent, DALT, and 5-MeO-DMT. In embodiments, the combination comprises moxonidine, a CB₁ agent, DALT, and 5-MeO-MiPT In embodiments, the combination comprises moxonidine, a CB₁ agent, DALT, and AGH-192. In embodiments, the combination comprises moxonidine, a CB₁ agent, DALT, and AGH-107. In embodiments, the combination comprises moxonidine, a CB₁ agent, DALT, and AMT. In embodiments, the combination comprises moxonidine, a CB₁ agent, DALT, and aripiprazole. In embodiments, the combination comprises moxonidine, a CB₁ agent, DALT, and AS-19. In embodiments, the combination comprises moxonidine, a CB₁ agent, DALT, and E-55888. In embodiments, the combination comprises moxonidine, a CB₁ agent, DALT, and LSD. In embodiments, the combination comprises moxonidine, a CB₁ agent, DALT, and LP-211. In embodiments, the combination comprises moxonidine, a CB₁ agent, DALT, and LP-44.

In embodiments, the combination comprises moxonidine, a CB₁ agent, DOB, and 5-CT. In embodiments, the combination comprises moxonidine, a CB₁ agent, DOB, and 5-MeO-DALT. In embodiments, the combination comprises moxonidine, a CB₁ agent, DOB, and 5-MeO-DMT. In embodiments, the combination comprises moxonidine, a CB₁ agent, DOB, and 5-MeO-MiPT In embodiments, the combination comprises moxonidine, a CB₁ agent, DOB, and AGH-192. In embodiments, the combination comprises moxonidine, a CB₁ agent, DOB, and AGH-107. In embodiments, the combination comprises moxonidine, a CB₁ agent, DOB, and AMT. In embodiments, the combination comprises moxonidine, a CB₁ agent, DOB, and aripiprazole. In embodiments, the combination comprises moxonidine, a CB₁ agent, DOB, and AS-19. In embodiments, the combination comprises moxonidine, a CB₁ agent, DOB, and E-55888. In embodiments, the combination comprises moxonidine, a CB₁ agent, DOB, and LSD. In embodiments, the combination comprises moxonidine, a CB₁ agent, DOB, and LP-211. In embodiments, the combination comprises moxonidine, a CB₁ agent, DOB, and LP-44.

In embodiments, the combination comprises moxonidine, a CB₁ agent, DOET, and 5-CT. In embodiments, the combination comprises moxonidine, a CB₁ agent, DOET, and 5-MeO-DALT. In embodiments, the combination comprises moxonidine, a CB₁ agent, DOET, and 5-MeO-DMT In embodiments, the combination comprises moxonidine, a CB₁ agent, DOET, and 5-MeO-MiPT. In embodiments, the combination comprises moxonidine, a CB₁ agent, DOET, and AGH-192. In embodiments, the combination comprises moxonidine, a CB₁ agent, DOET, and AGH-107. In embodiments, the combination comprises moxonidine, a CB₁ agent, DOET, and AMT. In embodiments, the combination comprises moxonidine, a CB₁ agent, DOET, and aripiprazole. In embodiments, the combination comprises moxonidine, a CB₁ agent, DOET, and AS-19. In embodiments, the combination comprises moxonidine, a CB₁ agent, DOET, and E-55888. In embodiments, the combination comprises moxonidine, a CB₁ agent, DOET, and LSD. In embodiments, the combination comprises moxonidine, a CB₁ agent, DOET, and LP-211. In embodiments, the combination comprises moxonidine, a CB₁ agent, DOET, and LP-44.

In embodiments, the combination comprises moxonidine, a CB₁ agent, DOHx, and 5-CT. In embodiments, the combination comprises moxonidine, a CB₁ agent, DOHx, and 5-MeO-DALT. In embodiments, the combination comprises moxonidine, a CB₁ agent, DOHx, and 5-MeO-DMT In embodiments, the combination comprises moxonidine, a CB₁ agent, DOHx, and 5-MeO-MiPT In embodiments, the combination comprises moxonidine, a CB₁ agent, DOHx, and AGH-192. In embodiments, the combination comprises moxonidine, a CB₁ agent, DOHx, and AGH-107. In embodiments, the combination comprises moxonidine, a CB₁ agent, DOHx, and AMT. In embodiments, the combination comprises moxonidine, a CB₁ agent, DOHx, and aripiprazole. In embodiments, the combination comprises moxonidine, a CB₁ agent, DOHx, and AS-19. In embodiments, the combination comprises moxonidine, a CB₁ agent, DOHx, and E-55888. In embodiments, the combination comprises moxonidine, a CB₁ agent, DOHx, and LSD. In embodiments, the combination comprises moxonidine, a CB₁ agent, DOHx, and LP-211. In embodiments, the combination comprises moxonidine, a CB₁ agent, DOHx, and LP-44.

In embodiments, the combination comprises moxonidine, a CB₁ agent, MEM, and 5-CT. In embodiments, the combination comprises moxonidine, a CB₁ agent, MEM, and 5-MeO-DALT. In embodiments, the combination comprises moxonidine, a CB₁ agent, MEM, and 5-MeO-DMT In embodiments, the combination comprises moxonidine, a CB₁ agent, MEM, and 5-MeO-MiPT. In embodiments, the combination comprises moxonidine, a CB₁ agent, MEM, and AGH-192. In embodiments, the combination comprises moxonidine, a CB₁ agent, MEM, and AGH-107. In embodiments, the combination comprises moxonidine, a CB₁ agent, MEM, and AMT. In embodiments, the combination comprises moxonidine, a CB₁ agent, MEM, and aripiprazole. In embodiments, the combination comprises moxonidine, a CB₁ agent, MEM, and AS-19. In embodiments, the combination comprises moxonidine, a CB₁ agent, MEM, and E-55888. In embodiments, the combination comprises moxonidine, a CB₁ agent, MEM, and LSD. In embodiments, the combination comprises moxonidine, a CB₁ agent, MEM, and LP-211. In embodiments, the combination comprises moxonidine, a CB₁ agent, MEM, and LP-44.

In embodiments, the combination comprises moxonidine, a CB₁ agent, CPP, and 5-CT. In embodiments, the combination comprises moxonidine, a CB₁ agent, CPP, and 5-MeO-DALT. In embodiments, the combination comprises moxonidine, a CB₁ agent, CPP, and 5-MeO-DMT In embodiments, the combination comprises moxonidine, a CB₁ agent, CPP, and 5-MeO-MiPT. In embodiments, the combination comprises moxonidine, a CB₁ agent, CPP, and AGH-192. In embodiments, the combination comprises moxonidine, a CB₁ agent, CPP, and AGH-107. In embodiments, the combination comprises moxonidine, a CB₁ agent, CPP, and AMT. In embodiments, the combination comprises moxonidine, a CB₁ agent, CPP, and aripiprazole. In embodiments, the combination comprises moxonidine, a CB₁ agent, CPP, and AS-19. In embodiments, the combination comprises moxonidine, a CB₁ agent, CPP, and E-55888. In embodiments, the combination comprises moxonidine, a CB₁ agent, CPP, and LSD. In embodiments, the combination comprises moxonidine, a CB₁ agent, CPP, and LP-211. In embodiments, the combination comprises moxonidine, a CB₁ agent, CPP, and LP-44.

In embodiments, the combination comprises moxonidine, a CB₁ agent, NBOH-2C-CN, and 5-CT. In embodiments, the combination comprises moxonidine, a CB₁ agent, NBOH-2C-CN, and 5-MeO-DALT. In embodiments, the combination comprises moxonidine, a CB₁ agent, NBOH-2C-CN, and 5-MeO-DMT. In embodiments, the combination comprises moxonidine, a CB₁ agent, NBOH-2C-CN, and 5-MeO-MiPT. In embodiments, the combination comprises moxonidine, a CB₁ agent, NBOH-2C-CN, and AGH-192. In embodiments, the combination comprises moxonidine, a CB₁ agent, NBOH-2C-CN, and AGH-107. In embodiments, the combination comprises moxonidine, a CB₁ agent, NBOH-2C-CN, and AMT. In embodiments, the combination comprises moxonidine, a CB₁ agent, NBOH-2C-CN, and aripiprazole. In embodiments, the combination comprises moxonidine, a CB₁ agent, NBOH-2C-CN, and AS-19. In embodiments, the combination comprises moxonidine, a CB₁ agent, NBOH-2C-CN, and E-55888. In embodiments, the combination comprises moxonidine, a CB₁ agent, NBOH-2C-CN, and LSD. In embodiments, the combination comprises moxonidine, a CB₁ agent, NBOH-2C-CN, and LP-211. In embodiments, the combination comprises moxonidine, a CB₁ agent, NBOH-2C-CN, and LP-44.

In embodiments, the combination comprises moxonidine, a CB₁ agent, TCB-2, and 5-CT. In embodiments, the combination comprises moxonidine, a CB₁ agent, TCB-2, and 5-MeO-DALT. In embodiments, the combination comprises moxonidine, a CB₁ agent, TCB-2, and 5-MeO-DMT In embodiments, the combination comprises moxonidine, a CB₁ agent, TCB-2, and 5-MeO-MiPT. In embodiments, the combination comprises moxonidine, a CB₁ agent, TCB-2, and AGH-192. In embodiments, the combination comprises moxonidine, a CB₁ agent, TCB-2, and AGH-107. In embodiments, the combination comprises moxonidine, a CB₁ agent, TCB-2, and AMT. In embodiments, the combination comprises moxonidine, a CB₁ agent, TCB-2, and aripiprazole. In embodiments, the combination comprises moxonidine, a CB₁ agent, TCB-2, and AS-19. In embodiments, the combination comprises moxonidine, a CB₁ agent, TCB-2, and E-55888. In embodiments, the combination comprises moxonidine, a CB₁ agent, TCB-2, and LSD. In embodiments, the combination comprises moxonidine, a CB₁ agent, TCB-2, and LP-211. In embodiments, the combination comprises moxonidine, a CB₁ agent, TCB-2, and LP-44.

In embodiments, the combination comprises moxonidine, a CB₁ agent, WAY 161503, and 5-CT. In embodiments, the combination comprises moxonidine, a CB₁ agent, WAY 161503, and 5-MeO-DALT. In embodiments, the combination comprises moxonidine, a CB₁ agent, WAY 161503, and 5-MeO-DMT In embodiments, the combination comprises moxonidine, a CB₁ agent, WAY 161503, and 5-MeO-MiPT In embodiments, the combination comprises moxonidine, a CB₁ agent, WAY 161503, and AGH-192. In embodiments, the combination comprises moxonidine, a CB₁ agent, WAY 161503, and AGH-107. In embodiments, the combination comprises moxonidine, a CB₁ agent, WAY 161503, and AMT. In embodiments, the combination comprises moxonidine, a CB₁ agent, WAY 161503, and aripiprazole. In embodiments, the combination comprises moxonidine, a CB₁ agent, WAY 161503, and AS-19. In embodiments, the combination comprises moxonidine, a CB₁ agent, WAY 161503, and E-55888. In embodiments, the combination comprises moxonidine, a CB₁ agent, WAY 161503, and LSD. In embodiments, the combination comprises moxonidine, a CB₁ agent, WAY 161503, and LP-211. In embodiments, the combination comprises moxonidine, a CB₁ agent, WAY 161503, and LP-44.

In embodiments, the combination comprises naphazoline, a CB₁ agent, 1-methylpsilocin, and 5-CT. In embodiments, the combination comprises naphazoline, a CB₁ agent, 1-methylpsilocin, and 5-MeO-DALT. In embodiments, the combination comprises naphazoline, a CB₁ agent, 1-methylpsilocin, and 5-MeO-DMT. In embodiments, the combination comprises naphazoline, a CB₁ agent, 1-methylpsilocin, and 5-MeO-MiPT. In embodiments, the combination comprises naphazoline, a CB₁ agent, 1-methylpsilocin, and AGH-192. In embodiments, the combination comprises naphazoline, a CB₁ agent, 1-methylpsilocin, and AGH-107. In embodiments, the combination comprises naphazoline, a CB₁ agent, 1-methylpsilocin, and AMT. In embodiments, the combination comprises naphazoline, a CB₁ agent, 1-methylpsilocin, and aripiprazole. In embodiments, the combination comprises naphazoline, a CB₁ agent, 1-methylpsilocin, and AS-19. In embodiments, the combination comprises naphazoline, a CB₁ agent, 1-methylpsilocin, and E-55888. In embodiments, the combination comprises naphazoline, a CB₁ agent, 1-methylpsilocin, and LSD. In embodiments, the combination comprises naphazoline, a CB₁ agent, 1-methylpsilocin, and LP-211. In embodiments, the combination comprises naphazoline, a CB₁ agent, 1-methylpsilocin, and LP-44.

In embodiments, the combination comprises naphazoline, a CB₁ agent, 2C-B-FLY, and 5-CT. In embodiments, the combination comprises naphazoline, a CB₁ agent, 2C-B-FLY, and 5-MeO-DALT. In embodiments, the combination comprises naphazoline, a CB₁ agent, 2C-B-FLY, and 5-MeO-DMT. In embodiments, the combination comprises naphazoline, a CB₁ agent, 2C-B-FLY, and 5-MeO-MiPT In embodiments, the combination comprises naphazoline, a CB₁ agent, 2C-B-FLY, and AGH-192. In embodiments, the combination comprises naphazoline, a CB₁ agent, 2C-B-FLY, and AGH-107. In embodiments, the combination comprises naphazoline, a CB₁ agent, 2C-B-FLY, and AMT. In embodiments, the combination comprises naphazoline, a CB₁ agent, 2C-B-FLY, and aripiprazole. In embodiments, the combination comprises naphazoline, a CB₁ agent, 2C-B-FLY, and AS-19. In embodiments, the combination comprises naphazoline, a CB₁ agent, 2C-B-FLY, and E-55888. In embodiments, the combination comprises naphazoline, a CB₁ agent, 2C-B-FLY, and LSD. In embodiments, the combination comprises naphazoline, a CB₁ agent, 2C-B-FLY, and LP-211. In embodiments, the combination comprises naphazoline, a CB₁ agent, 2C-B-FLY, and LP-44.

In embodiments, the combination comprises naphazoline, a CB₁ agent, 5-MeO-AMT, and 5-CT. In embodiments, the combination comprises naphazoline, a CB₁ agent, 5-MeO-AMT, and 5-MeO-DALT. In embodiments, the combination comprises naphazoline, a CB₁ agent, 5-MeO-AMT, and 5-MeO-DMT In embodiments, the combination comprises naphazoline, a CB₁ agent, 5-MeO-AMT, and 5-MeO-MiPT. In embodiments, the combination comprises naphazoline, a CB₁ agent, 5-MeO-AMT, and AGH-192. In embodiments, the combination comprises naphazoline, a CB₁ agent, 5-MeO-AMT, and AGH-107. In embodiments, the combination comprises naphazoline, a CB₁ agent, 5-MeO-AMT, and AMT. In embodiments, the combination comprises naphazoline, a CB₁ agent, 5-MeO-AMT, and aripiprazole. In embodiments, the combination comprises naphazoline, a CB₁ agent, 5-MeO-AMT, and AS-19. In embodiments, the combination comprises naphazoline, a CB₁ agent, 5-MeO-AMT, and E-55888. In embodiments, the combination comprises naphazoline, a CB₁ agent, 5-MeO-AMT, and LSD. In embodiments, the combination comprises naphazoline, a CB₁ agent, 5-MeO-AMT, and LP-211. In embodiments, the combination comprises naphazoline, a CB₁ agent, 5-MeO-AMT, and LP-44.

In embodiments, the combination comprises naphazoline, a CB₁ agent, AL-37350A, and 5-CT. In embodiments, the combination comprises naphazoline, a CB₁ agent, AL-37350A, and 5-MeO-DALT. In embodiments, the combination comprises naphazoline, a CB₁ agent, AL-37350A, and 5-MeO-DMT. In embodiments, the combination comprises naphazoline, a CB₁ agent, AL-37350A, and 5-MeO-MiPT In embodiments, the combination comprises naphazoline, a CB₁ agent, AL-37350A, and AGH-192. In embodiments, the combination comprises naphazoline, a CB₁ agent, AL-37350A, and AGH-107. In embodiments, the combination comprises naphazoline, a CB₁ agent, AL-37350A, and AMT. In embodiments, the combination comprises naphazoline, a CB₁ agent, AL-37350A, and aripiprazole. In embodiments, the combination comprises naphazoline, a CB₁ agent, AL-37350A, and AS-19. In embodiments, the combination comprises naphazoline, a CB₁ agent, AL-37350A, and E-55888. In embodiments, the combination comprises naphazoline, a CB₁ agent, AL-37350A, and LSD. In embodiments, the combination comprises naphazoline, a CB₁ agent, AL-37350A, and LP-211. In embodiments, the combination comprises naphazoline, a CB₁ agent, AL-37350A, and LP-44.

In embodiments, the combination comprises naphazoline, a CB₁ agent, CP 809101, and 5-CT. In embodiments, the combination comprises naphazoline, a CB₁ agent, CP 809101, and 5-MeO-DALT. In embodiments, the combination comprises naphazoline, a CB₁ agent, CP 809101, and 5-MeO-DMT. In embodiments, the combination comprises naphazoline, a CB₁ agent, CP 809101, and 5-MeO-MiPT In embodiments, the combination comprises naphazoline, a CB₁ agent, CP 809101, and AGH-192. In embodiments, the combination comprises naphazoline, a CB₁ agent, CP 809101, and AGH-107. In embodiments, the combination comprises naphazoline, a CB₁ agent, CP 809101, and AMT. In embodiments, the combination comprises naphazoline, a CB₁ agent, CP 809101, and aripiprazole. In embodiments, the combination comprises naphazoline, a CB₁ agent, CP 809101, and AS-19. In embodiments, the combination comprises naphazoline, a CB₁ agent, CP 809101, and E-55888. In embodiments, the combination comprises naphazoline, a CB₁ agent, CP 809101, and LSD. In embodiments, the combination comprises naphazoline, a CB₁ agent, CP 809101, and LP-211. In embodiments, the combination comprises naphazoline, a CB₁ agent, CP 809101, and LP-44.

In embodiments, the combination comprises naphazoline, a CB₁ agent, DALT, and 5-CT. In embodiments, the combination comprises naphazoline, a CB₁ agent, DALT, and 5-MeO-DALT. In embodiments, the combination comprises naphazoline, a CB₁ agent, DALT, and 5-MeO-DMT. In embodiments, the combination comprises naphazoline, a CB₁ agent, DALT, and 5-MeO-MiPT In embodiments, the combination comprises naphazoline, a CB₁ agent, DALT, and AGH-192. In embodiments, the combination comprises naphazoline, a CB₁ agent, DALT, and AGH-107. In embodiments, the combination comprises naphazoline, a CB₁ agent, DALT, and AMT. In embodiments, the combination comprises naphazoline, a CB₁ agent, DALT, and aripiprazole. In embodiments, the combination comprises naphazoline, a CB₁ agent, DALT, and AS-19. In embodiments, the combination comprises naphazoline, a CB₁ agent, DALT, and E-55888. In embodiments, the combination comprises naphazoline, a CB₁ agent, DALT, and LSD. In embodiments, the combination comprises naphazoline, a CB₁ agent, DALT, and LP-211. In embodiments, the combination comprises naphazoline, a CB₁ agent, DALT, and LP-44.

In embodiments, the combination comprises naphazoline, a CB₁ agent, DOB, and 5-CT. In embodiments, the combination comprises naphazoline, a CB₁ agent, DOB, and 5-MeO-DALT. In embodiments, the combination comprises naphazoline, a CB₁ agent, DOB, and 5-MeO-DMT. In embodiments, the combination comprises naphazoline, a CB₁ agent, DOB, and 5-MeO-MiPT In embodiments, the combination comprises naphazoline, a CB₁ agent, DOB, and AGH-192. In embodiments, the combination comprises naphazoline, a CB₁ agent, DOB, and AGH-107. In embodiments, the combination comprises naphazoline, a CB₁ agent, DOB, and AMT. In embodiments, the combination comprises naphazoline, a CB₁ agent, DOB, and aripiprazole. In embodiments, the combination comprises naphazoline, a CB₁ agent, DOB, and AS-19. In embodiments, the combination comprises naphazoline, a CB₁ agent, DOB, and E-55888. In embodiments, the combination comprises naphazoline, a CB₁ agent, DOB, and LSD. In embodiments, the combination comprises naphazoline, a CB₁ agent, DOB, and LP-211. In embodiments, the combination comprises naphazoline, a CB₁ agent, DOB, and LP-44.

In embodiments, the combination comprises naphazoline, a CB₁ agent, DOET, and 5-CT. In embodiments, the combination comprises naphazoline, a CB₁ agent, DOET, and 5-MeO-DALT. In embodiments, the combination comprises naphazoline, a CB₁ agent, DOET, and 5-MeO-DMT In embodiments, the combination comprises naphazoline, a CB₁ agent, DOET, and 5-MeO-MiPT. In embodiments, the combination comprises naphazoline, a CB₁ agent, DOET, and AGH-192. In embodiments, the combination comprises naphazoline, a CB₁ agent, DOET, and AGH-107. In embodiments, the combination comprises naphazoline, a CB₁ agent, DOET, and AMT. In embodiments, the combination comprises naphazoline, a CB₁ agent, DOET, and aripiprazole. In embodiments, the combination comprises naphazoline, a CB₁ agent, DOET, and AS-19. In embodiments, the combination comprises naphazoline, a CB₁ agent, DOET, and E-55888. In embodiments, the combination comprises naphazoline, a CB₁ agent, DOET, and LSD. In embodiments, the combination comprises naphazoline, a CB₁ agent, DOET, and LP-211. In embodiments, the combination comprises naphazoline, a CB₁ agent, DOET, and LP-44.

In embodiments, the combination comprises naphazoline, a CB₁ agent, DOHx, and 5-CT. In embodiments, the combination comprises naphazoline, a CB₁ agent, DOHx, and 5-MeO-DALT. In embodiments, the combination comprises naphazoline, a CB₁ agent, DOHx, and 5-MeO-DMT. In embodiments, the combination comprises naphazoline, a CB₁ agent, DOHx, and 5-MeO-MiPT In embodiments, the combination comprises naphazoline, a CB₁ agent, DOHx, and AGH-192. In embodiments, the combination comprises naphazoline, a CB₁ agent, DOHx, and AGH-107. In embodiments, the combination comprises naphazoline, a CB₁ agent, DOHx, and AMT. In embodiments, the combination comprises naphazoline, a CB₁ agent, DOHx, and aripiprazole. In embodiments, the combination comprises naphazoline, a CB₁ agent, DOHx, and AS-19. In embodiments, the combination comprises naphazoline, a CB₁ agent, DOHx, and E-55888. In embodiments, the combination comprises naphazoline, a CB₁ agent, DOHx, and LSD. In embodiments, the combination comprises naphazoline, a CB₁ agent, DOHx, and LP-211. In embodiments, the combination comprises naphazoline, a CB₁ agent, DOHx, and LP-44.

In embodiments, the combination comprises naphazoline, a CB₁ agent, MEM, and 5-CT. In embodiments, the combination comprises naphazoline, a CB₁ agent, MEM, and 5-MeO-DALT. In embodiments, the combination comprises naphazoline, a CB₁ agent, MEM, and 5-MeO-DMT In embodiments, the combination comprises naphazoline, a CB₁ agent, MEM, and 5-MeO-MiPT. In embodiments, the combination comprises naphazoline, a CB₁ agent, MEM, and AGH-192. In embodiments, the combination comprises naphazoline, a CB₁ agent, MEM, and AGH-107. In embodiments, the combination comprises naphazoline, a CB₁ agent, MEM, and AMT. In embodiments, the combination comprises naphazoline, a CB₁ agent, MEM, and aripiprazole. In embodiments, the combination comprises naphazoline, a CB₁ agent, MEM, and AS-19. In embodiments, the combination comprises naphazoline, a CB₁ agent, MEM, and E-55888. In embodiments, the combination comprises naphazoline, a CB₁ agent, MEM, and LSD. In embodiments, the combination comprises naphazoline, a CB₁ agent, MEM, and LP-211. In embodiments, the combination comprises naphazoline, a CB₁ agent, MEM, and LP-44.

In embodiments, the combination comprises naphazoline, a CB₁ agent, CPP, and 5-CT. In embodiments, the combination comprises naphazoline, a CB₁ agent, CPP, and 5-MeO-DALT. In embodiments, the combination comprises naphazoline, a CB₁ agent, CPP, and 5-MeO-DMT In embodiments, the combination comprises naphazoline, a CB₁ agent, CPP, and 5-MeO-MiPT. In embodiments, the combination comprises naphazoline, a CB₁ agent, CPP, and AGH-192. In embodiments, the combination comprises naphazoline, a CB₁ agent, CPP, and AGH-107. In embodiments, the combination comprises naphazoline, a CB₁ agent, CPP, and AMT. In embodiments, the combination comprises naphazoline, a CB₁ agent, CPP, and aripiprazole. In embodiments, the combination comprises naphazoline, a CB₁ agent, CPP, and AS-19. In embodiments, the combination comprises naphazoline, a CB₁ agent, CPP, and E-55888. In embodiments, the combination comprises naphazoline, a CB₁ agent, CPP, and LSD. In embodiments, the combination comprises naphazoline, a CB₁ agent, CPP, and LP-211. In embodiments, the combination comprises naphazoline, a CB₁ agent, CPP, and LP-44.

In embodiments, the combination comprises naphazoline, a CB₁ agent, NBOH-2C-CN, and 5-CT. In embodiments, the combination comprises naphazoline, a CB₁ agent, NBOH-2C-CN, and 5-MeO-DALT. In embodiments, the combination comprises naphazoline, a CB₁ agent, NBOH-2C-CN, and 5-MeO-DMT. In embodiments, the combination comprises naphazoline, a CB₁ agent, NBOH-2C-CN, and 5-MeO-MiPT. In embodiments, the combination comprises naphazoline, a CB₁ agent, NBOH-2C-CN, and AGH-192. In embodiments, the combination comprises naphazoline, a CB₁ agent, NBOH-2C-CN, and AGH-107. In embodiments, the combination comprises naphazoline, a CB₁ agent, NBOH-2C-CN, and AMT. In embodiments, the combination comprises naphazoline, a CB₁ agent, NBOH-2C-CN, and aripiprazole. In embodiments, the combination comprises naphazoline, a CB₁ agent, NBOH-2C-CN, and AS-19. In embodiments, the combination comprises naphazoline, a CB₁ agent, NBOH-2C-CN, and E-55888. In embodiments, the combination comprises naphazoline, a CB₁ agent, NBOH-2C-CN, and LSD. In embodiments, the combination comprises naphazoline, a CB₁ agent, NBOH-2C-CN, and LP-211. In embodiments, the combination comprises naphazoline, a CB₁ agent, NBOH-2C-CN, and LP-44.

In embodiments, the combination comprises naphazoline, a CB₁ agent, TCB-2, and 5-CT. In embodiments, the combination comprises naphazoline, a CB₁ agent, TCB-2, and 5-MeO-DALT. In embodiments, the combination comprises naphazoline, a CB₁ agent, TCB-2, and 5-MeO-DMT In embodiments, the combination comprises naphazoline, a CB₁ agent, TCB-2, and 5-MeO-MiPT. In embodiments, the combination comprises naphazoline, a CB₁ agent, TCB-2, and AGH-192. In embodiments, the combination comprises naphazoline, a CB₁ agent, TCB-2, and AGH-107. In embodiments, the combination comprises naphazoline, a CB₁ agent, TCB-2, and AMT. In embodiments, the combination comprises naphazoline, a CB₁ agent, TCB-2, and aripiprazole. In embodiments, the combination comprises naphazoline, a CB₁ agent, TCB-2, and AS-19. In embodiments, the combination comprises naphazoline, a CB₁ agent, TCB-2, and E-55888. In embodiments, the combination comprises naphazoline, a CB₁ agent, TCB-2, and LSD. In embodiments, the combination comprises naphazoline, a CB₁ agent, TCB-2, and LP-211. In embodiments, the combination comprises naphazoline, a CB₁ agent, TCB-2, and LP-44.

In embodiments, the combination comprises naphazoline, a CB₁ agent, WAY 161503, and 5-CT. In embodiments, the combination comprises naphazoline, a CB₁ agent, WAY 161503, and 5-MeO-DALT In embodiments, the combination comprises naphazoline, a CB₁ agent, WAY 161503, and 5-MeO-DMT. In embodiments, the combination comprises naphazoline, a CB₁ agent, WAY 161503, and 5-MeO-MiPT In embodiments, the combination comprises naphazoline, a CB₁ agent, WAY 161503, and AGH-192. In embodiments, the combination comprises naphazoline, a CB₁ agent, WAY 161503, and AGH-107. In embodiments, the combination comprises naphazoline, a CB₁ agent, WAY 161503, and AMT. In embodiments, the combination comprises naphazoline, a CB₁ agent, WAY 161503, and aripiprazole. In embodiments, the combination comprises naphazoline, a CB₁ agent, WAY 161503, and AS-19. In embodiments, the combination comprises naphazoline, a CB₁ agent, WAY 161503, and E-55888. In embodiments, the combination comprises naphazoline, a CB₁ agent, WAY 161503, and LSD. In embodiments, the combination comprises naphazoline, a CB₁ agent, WAY 161503, and LP-211. In embodiments, the combination comprises naphazoline, a CB₁ agent, WAY 161503, and LP-44.

In embodiments, the combination comprises oxymetazoline, a CB₁ agent, 1-methylpsilocin, and 5-CT. In embodiments, the combination comprises oxymetazoline, a CB₁ agent, 1-methylpsilocin, and 5-MeO-DALT. In embodiments, the combination comprises oxymetazoline, a CB₁ agent, 1-methylpsilocin, and 5-MeO-DMT. In embodiments, the combination comprises oxymetazoline, a CB₁ agent, 1-methylpsilocin, and 5-MeO-MiPT In embodiments, the combination comprises oxymetazoline, a CB₁ agent, 1-methylpsilocin, and AGH-192. In embodiments, the combination comprises oxymetazoline, a CB₁ agent, 1-methylpsilocin, and AGH-107. In embodiments, the combination comprises oxymetazoline, a CB₁ agent, 1-methylpsilocin, and AMT. In embodiments, the combination comprises oxymetazoline, a CB₁ agent, 1-methylpsilocin, and aripiprazole. In embodiments, the combination comprises oxymetazoline, a CB₁ agent, 1-methylpsilocin, and AS-19. In embodiments, the combination comprises oxymetazoline, a CB₁ agent, 1-methylpsilocin, and E-55888. In embodiments, the combination comprises oxymetazoline, a CB₁ agent, 1-methylpsilocin, and LSD. In embodiments, the combination comprises oxymetazoline, a CB₁ agent, 1-methylpsilocin, and LP-211. In embodiments, the combination comprises oxymetazoline, a CB₁ agent, 1-methylpsilocin, and LP-44.

In embodiments, the combination comprises oxymetazoline, a CB₁ agent, 2C-B-FLY, and 5-CT. In embodiments, the combination comprises oxymetazoline, a CB₁ agent, 2C-B-FLY, and 5-MeO-DALT. In embodiments, the combination comprises oxymetazoline, a CB₁ agent, 2C-B-FLY, and 5-MeO-DMT In embodiments, the combination comprises oxymetazoline, a CB₁ agent, 2C-B-FLY, and 5-MeO-MiPT. In embodiments, the combination comprises oxymetazoline, a CB₁ agent, 2C-B-FLY, and AGH-192. In embodiments, the combination comprises oxymetazoline, a CB₁ agent, 2C-B-FLY, and AGH-107. In embodiments, the combination comprises oxymetazoline, a CB₁ agent, 2C-B-FLY, and AMT. In embodiments, the combination comprises oxymetazoline, a CB₁ agent, 2C-B-FLY, and aripiprazole. In embodiments, the combination comprises oxymetazoline, a CB₁ agent, 2C-B-FLY, and AS-19. In embodiments, the combination comprises oxymetazoline, a CB₁ agent, 2C-B-FLY, and E-55888. In embodiments, the combination comprises oxymetazoline, a CB₁ agent, 2C-B-FLY, and LSD. In embodiments, the combination comprises oxymetazoline, a CB₁ agent, 2C-B-FLY, and LP-211. In embodiments, the combination comprises oxymetazoline, a CB₁ agent, 2C-B-FLY, and LP-44.

In embodiments, the combination comprises oxymetazoline, a CB₁ agent, 5-MeO-AMT, and 5-CT. In embodiments, the combination comprises oxymetazoline, a CB₁ agent, 5-MeO-AMT, and 5-MeO-DALT. In embodiments, the combination comprises oxymetazoline, a CB₁ agent, 5-MeO-AMT, and 5-MeO-DMT. In embodiments, the combination comprises oxymetazoline, a CB₁ agent, 5-MeO-AMT, and 5-MeO-MiPT. In embodiments, the combination comprises oxymetazoline, a CB₁ agent, 5-MeO-AMT, and AGH-192. In embodiments, the combination comprises oxymetazoline, a CB₁ agent, 5-MeO-AMT, and AGH-107. In embodiments, the combination comprises oxymetazoline, a CB₁ agent, 5-MeO-AMT, and AMT. In embodiments, the combination comprises oxymetazoline, a CB₁ agent, 5-MeO-AMT, and aripiprazole. In embodiments, the combination comprises oxymetazoline, a CB₁ agent, 5-MeO-AMT, and AS-19. In embodiments, the combination comprises oxymetazoline, a CB₁ agent, 5-MeO-AMT, and E-55888. In embodiments, the combination comprises oxymetazoline, a CB₁ agent, 5-MeO-AMT, and LSD. In embodiments, the combination comprises oxymetazoline, a CB₁ agent, 5-MeO-AMT, and LP-211. In embodiments, the combination comprises oxymetazoline, a CB₁ agent, 5-MeO-AMT, and LP-44.

In embodiments, the combination comprises oxymetazoline, a CB₁ agent, AL-37350A, and 5-CT. In embodiments, the combination comprises oxymetazoline, a CB₁ agent, AL-37350A, and 5-MeO-DALT. In embodiments, the combination comprises oxymetazoline, a CB₁ agent, AL-37350A, and 5-MeO-DMT In embodiments, the combination comprises oxymetazoline, a CB₁ agent, AL-37350A, and 5-MeO-MiPT. In embodiments, the combination comprises oxymetazoline, a CB₁ agent, AL-37350A, and AGH-192. In embodiments, the combination comprises oxymetazoline, a CB₁ agent, AL-37350A, and AGH-107. In embodiments, the combination comprises oxymetazoline, a CB₁ agent, AL-37350A, and AMT. In embodiments, the combination comprises oxymetazoline, a CB₁ agent, AL-37350A, and aripiprazole. In embodiments, the combination comprises oxymetazoline, a CB₁ agent, AL-37350A, and AS-19. In embodiments, the combination comprises oxymetazoline, a CB₁ agent, AL-37350A, and E-55888. In embodiments, the combination comprises oxymetazoline, a CB₁ agent, AL-37350A, and LSD. In embodiments, the combination comprises oxymetazoline, a CB₁ agent, AL-37350A, and LP-211. In embodiments, the combination comprises oxymetazoline, a CB₁ agent, AL-37350A, and LP-44.

In embodiments, the combination comprises oxymetazoline, a CB₁ agent, CP 809101, and 5-CT. In embodiments, the combination comprises oxymetazoline, a CB₁ agent, CP 809101, and 5-MeO-DALT. In embodiments, the combination comprises oxymetazoline, a CB₁ agent, CP 809101, and 5-MeO-DMT In embodiments, the combination comprises oxymetazoline, a CB₁ agent, CP 809101, and 5-MeO-MiPT. In embodiments, the combination comprises oxymetazoline, a CB₁ agent, CP 809101, and AGH-192. In embodiments, the combination comprises oxymetazoline, a CB₁ agent, CP 809101, and AGH-107. In embodiments, the combination comprises oxymetazoline, a CB₁ agent, CP 809101, and AMT. In embodiments, the combination comprises oxymetazoline, a CB₁ agent, CP 809101, and aripiprazole. In embodiments, the combination comprises oxymetazoline, a CB₁ agent, CP 809101, and AS-19. In embodiments, the combination comprises oxymetazoline, a CB₁ agent, CP 809101, and E-55888. In embodiments, the combination comprises oxymetazoline, a CB₁ agent, CP 809101, and LSD. In embodiments, the combination comprises oxymetazoline, a CB₁ agent, CP 809101, and LP-211. In embodiments, the combination comprises oxymetazoline, a CB₁ agent, CP 809101, and LP-44.

In embodiments, the combination comprises oxymetazoline, a CB₁ agent, DALT, and 5-CT. In embodiments, the combination comprises oxymetazoline, a CB₁ agent, DALT, and 5-MeO-DALT. In embodiments, the combination comprises oxymetazoline, a CB₁ agent, DALT, and 5-MeO-DMT In embodiments, the combination comprises oxymetazoline, a CB₁ agent, DALT, and 5-MeO-MiPT. In embodiments, the combination comprises oxymetazoline, a CB₁ agent, DALT, and AGH-192. In embodiments, the combination comprises oxymetazoline, a CB₁ agent, DALT, and AGH-107. In embodiments, the combination comprises oxymetazoline, a CB₁ agent, DALT, and AMT. In embodiments, the combination comprises oxymetazoline, a CB₁ agent, DALT, and aripiprazole. In embodiments, the combination comprises oxymetazoline, a CB₁ agent, DALT, and AS-19. In embodiments, the combination comprises oxymetazoline, a CB₁ agent, DALT, and E-55888. In embodiments, the combination comprises oxymetazoline, a CB₁ agent, DALT, and LSD. In embodiments, the combination comprises oxymetazoline, a CB₁ agent, DALT, and LP-211. In embodiments, the combination comprises oxymetazoline, a CB₁ agent, DALT, and LP-44.

In embodiments, the combination comprises oxymetazoline, a CB₁ agent, DOB, and 5-CT. In embodiments, the combination comprises oxymetazoline, a CB₁ agent, DOB, and 5-MeO-DALT. In embodiments, the combination comprises oxymetazoline, a CB₁ agent, DOB, and 5-MeO-DMT In embodiments, the combination comprises oxymetazoline, a CB₁ agent, DOB, and 5-MeO-MiPT. In embodiments, the combination comprises oxymetazoline, a CB₁ agent, DOB, and AGH-192. In embodiments, the combination comprises oxymetazoline, a CB₁ agent, DOB, and AGH-107. In embodiments, the combination comprises oxymetazoline, a CB₁ agent, DOB, and AMT. In embodiments, the combination comprises oxymetazoline, a CB₁ agent, DOB, and aripiprazole. In embodiments, the combination comprises oxymetazoline, a CB₁ agent, DOB, and AS-19. In embodiments, the combination comprises oxymetazoline, a CB₁ agent, DOB, and E-55888. In embodiments, the combination comprises oxymetazoline, a CB₁ agent, DOB, and LSD. In embodiments, the combination comprises oxymetazoline, a CB₁ agent, DOB, and LP-211. In embodiments, the combination comprises oxymetazoline, a CB₁ agent, DOB, and LP-44.

In embodiments, the combination comprises oxymetazoline, a CB₁ agent, DOET, and 5-CT. In embodiments, the combination comprises oxymetazoline, a CB₁ agent, DOET, and 5-MeO-DALT. In embodiments, the combination comprises oxymetazoline, a CB₁ agent, DOET, and 5-MeO-DMT. In embodiments, the combination comprises oxymetazoline, a CB₁ agent, DOET, and 5-MeO-MiPT. In embodiments, the combination comprises oxymetazoline, a CB₁ agent, DOET, and AGH-192. In embodiments, the combination comprises oxymetazoline, a CB₁ agent, DOET, and AGH-107. In embodiments, the combination comprises oxymetazoline, a CB₁ agent, DOET, and AMT. In embodiments, the combination comprises oxymetazoline, a CB₁ agent, DOET, and aripiprazole. In embodiments, the combination comprises oxymetazoline, a CB₁ agent, DOET, and AS-19. In embodiments, the combination comprises oxymetazoline, a CB₁ agent, DOET, and E-55888. In embodiments, the combination comprises oxymetazoline, a CB₁ agent, DOET, and LSD. In embodiments, the combination comprises oxymetazoline, a CB₁ agent, DOET, and LP-211. In embodiments, the combination comprises oxymetazoline, a CB₁ agent, DOET, and LP-44.

In embodiments, the combination comprises oxymetazoline, a CB₁ agent, DOHx, and 5-CT. In embodiments, the combination comprises oxymetazoline, a CB₁ agent, DOHx, and 5-MeO-DALT. In embodiments, the combination comprises oxymetazoline, a CB₁ agent, DOHx, and 5-MeO-DMT In embodiments, the combination comprises oxymetazoline, a CB₁ agent, DOHx, and 5-MeO-MiPT. In embodiments, the combination comprises oxymetazoline, a CB₁ agent, DOHx, and AGH-192. In embodiments, the combination comprises oxymetazoline, a CB₁ agent, DOHx, and AGH-107. In embodiments, the combination comprises oxymetazoline, a CB₁ agent, DOHx, and AMT. In embodiments, the combination comprises oxymetazoline, a CB₁ agent, DOHx, and aripiprazole. In embodiments, the combination comprises oxymetazoline, a CB₁ agent, DOHx, and AS-19. In embodiments, the combination comprises oxymetazoline, a CB₁ agent, DOHx, and E-55888. In embodiments, the combination comprises oxymetazoline, a CB₁ agent, DOHx, and LSD. In embodiments, the combination comprises oxymetazoline, a CB₁ agent, DOHx, and LP-211. In embodiments, the combination comprises oxymetazoline, a CB₁ agent, DOHx, and LP-44.

In embodiments, the combination comprises oxymetazoline, a CB₁ agent, MEM, and 5-CT. In embodiments, the combination comprises oxymetazoline, a CB₁ agent, MEM, and 5-MeO-DALT. In embodiments, the combination comprises oxymetazoline, a CB₁ agent, MEM, and 5-MeO-DMT. In embodiments, the combination comprises oxymetazoline, a CB₁ agent, MEM, and 5-MeO-MiPT In embodiments, the combination comprises oxymetazoline, a CB₁ agent, MEM, and AGH-192. In embodiments, the combination comprises oxymetazoline, a CB₁ agent, MEM, and AGH-107. In embodiments, the combination comprises oxymetazoline, a CB₁ agent, MEM, and AMT. In embodiments, the combination comprises oxymetazoline, a CB₁ agent, MEM, and aripiprazole. In embodiments, the combination comprises oxymetazoline, a CB₁ agent, MEM, and AS-19. In embodiments, the combination comprises oxymetazoline, a CB₁ agent, MEM, and E-55888. In embodiments, the combination comprises oxymetazoline, a CB₁ agent, MEM, and LSD. In embodiments, the combination comprises oxymetazoline, a CB₁ agent, MEM, and LP-211. In embodiments, the combination comprises oxymetazoline, a CB₁ agent, MEM, and LP-44.

In embodiments, the combination comprises oxymetazoline, a CB₁ agent, CPP, and 5-CT. In embodiments, the combination comprises oxymetazoline, a CB₁ agent, CPP, and 5-MeO-DALT. In embodiments, the combination comprises oxymetazoline, a CB₁ agent, CPP, and 5-MeO-DMT. In embodiments, the combination comprises oxymetazoline, a CB₁ agent, CPP, and 5-MeO-MiPT In embodiments, the combination comprises oxymetazoline, a CB₁ agent, CPP, and AGH-192. In embodiments, the combination comprises oxymetazoline, a CB₁ agent, CPP, and AGH-107. In embodiments, the combination comprises oxymetazoline, a CB₁ agent, CPP, and AMT. In embodiments, the combination comprises oxymetazoline, a CB₁ agent, CPP, and aripiprazole. In embodiments, the combination comprises oxymetazoline, a CB₁ agent, CPP, and AS-19. In embodiments, the combination comprises oxymetazoline, a CB₁ agent, CPP, and E-55888. In embodiments, the combination comprises oxymetazoline, a CB₁ agent, CPP, and LSD. In embodiments, the combination comprises oxymetazoline, a CB₁ agent, CPP, and LP-211. In embodiments, the combination comprises oxymetazoline, a CB₁ agent, CPP, and LP-44.

In embodiments, the combination comprises oxymetazoline, a CB₁ agent, NBOH-2C-CN, and 5-CT. In embodiments, the combination comprises oxymetazoline, a CB₁ agent, NBOH-2C-CN, and 5-MeO-DALT. In embodiments, the combination comprises oxymetazoline, a CB₁ agent, NBOH-2C-CN, and 5-MeO-DMT. In embodiments, the combination comprises oxymetazoline, a CB₁ agent, NBOH-2C-CN, and 5-MeO-MiPT. In embodiments, the combination comprises oxymetazoline, a CB₁ agent, NBOH-2C-CN, and AGH-192. In embodiments, the combination comprises oxymetazoline, a CB₁ agent, NBOH-2C-CN, and AGH-107. In embodiments, the combination comprises oxymetazoline, a CB₁ agent, NBOH-2C-CN, and AMT. In embodiments, the combination comprises oxymetazoline, a CB₁ agent, NBOH-2C-CN, and aripiprazole. In embodiments, the combination comprises oxymetazoline, a CB₁ agent, NBOH-2C-CN, and AS-19. In embodiments, the combination comprises oxymetazoline, a CB₁ agent, NBOH-2C-CN, and E-55888. In embodiments, the combination comprises oxymetazoline, a CB₁ agent, NBOH-2C-CN, and LSD. In embodiments, the combination comprises oxymetazoline, a CB₁ agent, NBOH-2C-CN, and LP-211. In embodiments, the combination comprises oxymetazoline, a CB₁ agent, NBOH-2C-CN, and LP-44.

In embodiments, the combination comprises oxymetazoline, a CB₁ agent, TCB-2, and 5-CT. In embodiments, the combination comprises oxymetazoline, a CB₁ agent, TCB-2, and 5-MeO-DALT. In embodiments, the combination comprises oxymetazoline, a CB₁ agent, TCB-2, and 5-MeO-DMT. In embodiments, the combination comprises oxymetazoline, a CB₁ agent, TCB-2, and 5-MeO-MiPT In embodiments, the combination comprises oxymetazoline, a CB₁ agent, TCB-2, and AGH-192. In embodiments, the combination comprises oxymetazoline, a CB₁ agent, TCB-2, and AGH-107. In embodiments, the combination comprises oxymetazoline, a CB₁ agent, TCB-2, and AMT. In embodiments, the combination comprises oxymetazoline, a CB₁ agent, TCB-2, and aripiprazole. In embodiments, the combination comprises oxymetazoline, a CB₁ agent, TCB-2, and AS-19. In embodiments, the combination comprises oxymetazoline, a CB₁ agent, TCB-2, and E-55888. In embodiments, the combination comprises oxymetazoline, a CB₁ agent, TCB-2, and LSD. In embodiments, the combination comprises oxymetazoline, a CB₁ agent, TCB-2, and LP-211. In embodiments, the combination comprises oxymetazoline, a CB₁ agent, TCB-2, and LP-44.

In embodiments, the combination comprises oxymetazoline, a CB₁ agent, WAY 161503, and 5-CT. In embodiments, the combination comprises oxymetazoline, a CB₁ agent, WAY 161503, and 5-MeO-DALT. In embodiments, the combination comprises oxymetazoline, a CB₁ agent, WAY 161503, and 5-MeO-DMT. In embodiments, the combination comprises oxymetazoline, a CB₁ agent, WAY 161503, and 5-MeO-MiPT. In embodiments, the combination comprises oxymetazoline, a CB₁ agent, WAY 161503, and AGH-192. In embodiments, the combination comprises oxymetazoline, a CB₁ agent, WAY 161503, and AGH-107. In embodiments, the combination comprises oxymetazoline, a CB₁ agent, WAY 161503, and AMT. In embodiments, the combination comprises oxymetazoline, a CB₁ agent, WAY 161503, and aripiprazole. In embodiments, the combination comprises oxymetazoline, a CB₁ agent, WAY 161503, and AS-19. In embodiments, the combination comprises oxymetazoline, a CB₁ agent, WAY 161503, and E-55888. In embodiments, the combination comprises oxymetazoline, a CB₁ agent, WAY 161503, and LSD. In embodiments, the combination comprises oxymetazoline, a CB₁ agent, WAY 161503, and LP-211. In embodiments, the combination comprises oxymetazoline, a CB₁ agent, WAY 161503, and LP-44.

In embodiments, the combination comprises rilmenidine, a CB₁ agent, 1-methylpsilocin, and 5-CT. In embodiments, the combination comprises rilmenidine, a CB₁ agent, 1-methylpsilocin, and 5-MeO-DALT. In embodiments, the combination comprises rilmenidine, a CB₁ agent, 1-methylpsilocin, and 5-MeO-DMT In embodiments, the combination comprises rilmenidine, a CB₁ agent, 1-methylpsilocin, and 5-MeO-MiPT. In embodiments, the combination comprises rilmenidine, a CB₁ agent, 1-methylpsilocin, and AGH-192. In embodiments, the combination comprises rilmenidine, a CB₁ agent, 1-methylpsilocin, and AGH-107. In embodiments, the combination comprises rilmenidine, a CB₁ agent, 1-methylpsilocin, and AMT. In embodiments, the combination comprises rilmenidine, a CB₁ agent, 1-methylpsilocin, and aripiprazole. In embodiments, the combination comprises rilmenidine, a CB₁ agent, 1-methylpsilocin, and AS-19. In embodiments, the combination comprises rilmenidine, a CB₁ agent, 1-methylpsilocin, and E-55888. In embodiments, the combination comprises rilmenidine, a CB₁ agent, 1-methylpsilocin, and LSD. In embodiments, the combination comprises rilmenidine, a CB₁ agent, 1-methylpsilocin, and LP-211. In embodiments, the combination comprises rilmenidine, a CB₁ agent, 1-methylpsilocin, and LP-44.

In embodiments, the combination comprises rilmenidine, a CB₁ agent, 2C-B-FLY, and 5-CT. In embodiments, the combination comprises rilmenidine, a CB₁ agent, 2C-B-FLY, and 5-MeO-DALT. In embodiments, the combination comprises rilmenidine, a CB₁ agent, 2C-B-FLY, and 5-MeO-DMT. In embodiments, the combination comprises rilmenidine, a CB₁ agent, 2C-B-FLY, and 5-MeO-MiPT In embodiments, the combination comprises rilmenidine, a CB₁ agent, 2C-B-FLY, and AGH-192. In embodiments, the combination comprises rilmenidine, a CB₁ agent, 2C-B-FLY, and AGH-107. In embodiments, the combination comprises rilmenidine, a CB₁ agent, 2C-B-FLY, and AMT. In embodiments, the combination comprises rilmenidine, a CB₁ agent, 2C-B-FLY, and aripiprazole. In embodiments, the combination comprises rilmenidine, a CB₁ agent, 2C-B-FLY, and AS-19. In embodiments, the combination comprises rilmenidine, a CB₁ agent, 2C-B-FLY, and E-55888. In embodiments, the combination comprises rilmenidine, a CB₁ agent, 2C-B-FLY, and LSD. In embodiments, the combination comprises rilmenidine, a CB₁ agent, 2C-B-FLY, and LP-211. In embodiments, the combination comprises rilmenidine, a CB₁ agent, 2C-B-FLY, and LP-44.

In embodiments, the combination comprises rilmenidine, a CB₁ agent, 5-MeO-AMT, and 5-CT. In embodiments, the combination comprises rilmenidine, a CB₁ agent, 5-MeO-AMT, and 5-MeO-DALT. In embodiments, the combination comprises rilmenidine, a CB₁ agent, 5-MeO-AMT, and 5-MeO-DMT. In embodiments, the combination comprises rilmenidine, a CB₁ agent, 5-MeO-AMT, and 5-MeO-MiPT. In embodiments, the combination comprises rilmenidine, a CB₁ agent, 5-MeO-AMT, and AGH-192. In embodiments, the combination comprises rilmenidine, a CB₁ agent, 5-MeO-AMT, and AGH-107. In embodiments, the combination comprises rilmenidine, a CB₁ agent, 5-MeO-AMT, and AMT. In embodiments, the combination comprises rilmenidine, a CB₁ agent, 5-MeO-AMT, and aripiprazole. In embodiments, the combination comprises rilmenidine, a CB₁ agent, 5-MeO-AMT, and AS-19. In embodiments, the combination comprises rilmenidine, a CB₁ agent, 5-MeO-AMT, and E-55888. In embodiments, the combination comprises rilmenidine, a CB₁ agent, 5-MeO-AMT, and LSD. In embodiments, the combination comprises rilmenidine, a CB₁ agent, 5-MeO-AMT, and LP-211. In embodiments, the combination comprises rilmenidine, a CB₁ agent, 5-MeO-AMT, and LP-44.

In embodiments, the combination comprises rilmenidine, a CB₁ agent, AL-37350A, and 5-CT. In embodiments, the combination comprises rilmenidine, a CB₁ agent, AL-37350A, and 5-MeO-DALT. In embodiments, the combination comprises rilmenidine, a CB₁ agent, AL-37350A, and 5-MeO-DMT. In embodiments, the combination comprises rilmenidine, a CB₁ agent, AL-37350A, and 5-MeO-MiPT In embodiments, the combination comprises rilmenidine, a CB₁ agent, AL-37350A, and AGH-192. In embodiments, the combination comprises rilmenidine, a CB₁ agent, AL-37350A, and AGH-107. In embodiments, the combination comprises rilmenidine, a CB₁ agent, AL-37350A, and AMT. In embodiments, the combination comprises rilmenidine, a CB₁ agent, AL-37350A, and aripiprazole. In embodiments, the combination comprises rilmenidine, a CB₁ agent, AL-37350A, and AS-19. In embodiments, the combination comprises rilmenidine, a CB₁ agent, AL-37350A, and E-55888. In embodiments, the combination comprises rilmenidine, a CB₁ agent, AL-37350A, and LSD. In embodiments, the combination comprises rilmenidine, a CB₁ agent, AL-37350A, and LP-211. In embodiments, the combination comprises rilmenidine, a CB₁ agent, AL-37350A, and LP-44.

In embodiments, the combination comprises rilmenidine, a CB₁ agent, CP 809101, and 5-CT. In embodiments, the combination comprises rilmenidine, a CB₁ agent, CP 809101, and 5-MeO-DALT. In embodiments, the combination comprises rilmenidine, a CB₁ agent, CP 809101, and 5-MeO-DMT. In embodiments, the combination comprises rilmenidine, a CB₁ agent, CP 809101, and 5-MeO-MiPT In embodiments, the combination comprises rilmenidine, a CB₁ agent, CP 809101, and AGH-192. In embodiments, the combination comprises rilmenidine, a CB₁ agent, CP 809101, and AGH-107. In embodiments, the combination comprises rilmenidine, a CB₁ agent, CP 809101, and AMT. In embodiments, the combination comprises rilmenidine, a CB₁ agent, CP 809101, and aripiprazole. In embodiments, the combination comprises rilmenidine, a CB₁ agent, CP 809101, and AS-19. In embodiments, the combination comprises rilmenidine, a CB₁ agent, CP 809101, and E-55888. In embodiments, the combination comprises rilmenidine, a CB₁ agent, CP 809101, and LSD. In embodiments, the combination comprises rilmenidine, a CB₁ agent, CP 809101, and LP-211. In embodiments, the combination comprises rilmenidine, a CB₁ agent, CP 809101, and LP-44.

In embodiments, the combination comprises rilmenidine, a CB₁ agent, DALT, and 5-CT. In embodiments, the combination comprises rilmenidine, a CB₁ agent, DALT, and 5-MeO-DALT. In embodiments, the combination comprises rilmenidine, a CB₁ agent, DALT, and 5-MeO-DMT. In embodiments, the combination comprises rilmenidine, a CB₁ agent, DALT, and 5-MeO-MiPT In embodiments, the combination comprises rilmenidine, a CB₁ agent, DALT, and AGH-192. In embodiments, the combination comprises rilmenidine, a CB₁ agent, DALT, and AGH-107. In embodiments, the combination comprises rilmenidine, a CB₁ agent, DALT, and AMT. In embodiments, the combination comprises rilmenidine, a CB₁ agent, DALT, and aripiprazole. In embodiments, the combination comprises rilmenidine, a CB₁ agent, DALT, and AS-19. In embodiments, the combination comprises rilmenidine, a CB₁ agent, DALT, and E-55888. In embodiments, the combination comprises rilmenidine, a CB₁ agent, DALT, and LSD. In embodiments, the combination comprises rilmenidine, a CB₁ agent, DALT, and LP-211. In embodiments, the combination comprises rilmenidine, a CB₁ agent, DALT, and LP-44.

In embodiments, the combination comprises rilmenidine, a CB₁ agent, DOB, and 5-CT. In embodiments, the combination comprises rilmenidine, a CB₁ agent, DOB, and 5-MeO-DALT. In embodiments, the combination comprises rilmenidine, a CB₁ agent, DOB, and 5-MeO-DMT. In embodiments, the combination comprises rilmenidine, a CB₁ agent, DOB, and 5-MeO-MiPT In embodiments, the combination comprises rilmenidine, a CB₁ agent, DOB, and AGH-192. In embodiments, the combination comprises rilmenidine, a CB₁ agent, DOB, and AGH-107. In embodiments, the combination comprises rilmenidine, a CB₁ agent, DOB, and AMT. In embodiments, the combination comprises rilmenidine, a CB₁ agent, DOB, and aripiprazole. In embodiments, the combination comprises rilmenidine, a CB₁ agent, DOB, and AS-19. In embodiments, the combination comprises rilmenidine, a CB₁ agent, DOB, and E-55888. In embodiments, the combination comprises rilmenidine, a CB₁ agent, DOB, and LSD. In embodiments, the combination comprises rilmenidine, a CB₁ agent, DOB, and LP-211. In embodiments, the combination comprises rilmenidine, a CB₁ agent, DOB, and LP-44.

In embodiments, the combination comprises rilmenidine, a CB₁ agent, DOET, and 5-CT. In embodiments, the combination comprises rilmenidine, a CB₁ agent, DOET, and 5-MeO-DALT. In embodiments, the combination comprises rilmenidine, a CB₁ agent, DOET, and 5-MeO-DMT In embodiments, the combination comprises rilmenidine, a CB₁ agent, DOET, and 5-MeO-MiPT. In embodiments, the combination comprises rilmenidine, a CB₁ agent, DOET, and AGH-192. In embodiments, the combination comprises rilmenidine, a CB₁ agent, DOET, and AGH-107. In embodiments, the combination comprises rilmenidine, a CB₁ agent, DOET, and AMT. In embodiments, the combination comprises rilmenidine, a CB₁ agent, DOET, and aripiprazole. In embodiments, the combination comprises rilmenidine, a CB₁ agent, DOET, and AS-19. In embodiments, the combination comprises rilmenidine, a CB₁ agent, DOET, and E-55888. In embodiments, the combination comprises rilmenidine, a CB₁ agent, DOET, and LSD. In embodiments, the combination comprises rilmenidine, a CB₁ agent, DOET, and LP-211. In embodiments, the combination comprises rilmenidine, a CB₁ agent, DOET, and LP-44.

In embodiments, the combination comprises rilmenidine, a CB₁ agent, DOHx, and 5-CT. In embodiments, the combination comprises rilmenidine, a CB₁ agent, DOHx, and 5-MeO-DALT. In embodiments, the combination comprises rilmenidine, a CB₁ agent, DOHx, and 5-MeO-DMT. In embodiments, the combination comprises rilmenidine, a CB₁ agent, DOHx, and 5-MeO-MiPT In embodiments, the combination comprises rilmenidine, a CB₁ agent, DOHx, and AGH-192. In embodiments, the combination comprises rilmenidine, a CB₁ agent, DOHx, and AGH-107. In embodiments, the combination comprises rilmenidine, a CB₁ agent, DOHx, and AMT. In embodiments, the combination comprises rilmenidine, a CB₁ agent, DOHx, and aripiprazole. In embodiments, the combination comprises rilmenidine, a CB₁ agent, DOHx, and AS-19. In embodiments, the combination comprises rilmenidine, a CB₁ agent, DOHx, and E-55888. In embodiments, the combination comprises rilmenidine, a CB₁ agent, DOHx, and LSD. In embodiments, the combination comprises rilmenidine, a CB₁ agent, DOHx, and LP-211. In embodiments, the combination comprises rilmenidine, a CB₁ agent, DOHx, and LP-44.

In embodiments, the combination comprises rilmenidine, a CB₁ agent, MEM, and 5-CT. In embodiments, the combination comprises rilmenidine, a CB₁ agent, MEM, and 5-MeO-DALT. In embodiments, the combination comprises rilmenidine, a CB₁ agent, MEM, and 5-MeO-DMT In embodiments, the combination comprises rilmenidine, a CB₁ agent, MEM, and 5-MeO-MiPT. In embodiments, the combination comprises rilmenidine, a CB₁ agent, MEM, and AGH-192. In embodiments, the combination comprises rilmenidine, a CB₁ agent, MEM, and AGH-107. In embodiments, the combination comprises rilmenidine, a CB₁ agent, MEM, and AMT. In embodiments, the combination comprises rilmenidine, a CB₁ agent, MEM, and aripiprazole. In embodiments, the combination comprises rilmenidine, a CB₁ agent, MEM, and AS-19. In embodiments, the combination comprises rilmenidine, a CB₁ agent, MEM, and E-55888. In embodiments, the combination comprises rilmenidine, a CB₁ agent, MEM, and LSD. In embodiments, the combination comprises rilmenidine, a CB₁ agent, MEM, and LP-211. In embodiments, the combination comprises rilmenidine, a CB₁ agent, MEM, and LP-44.

In embodiments, the combination comprises rilmenidine, a CB₁ agent, CPP, and 5-CT. In embodiments, the combination comprises rilmenidine, a CB₁ agent, CPP, and 5-MeO-DALT. In embodiments, the combination comprises rilmenidine, a CB₁ agent, CPP, and 5-MeO-DMT In embodiments, the combination comprises rilmenidine, a CB₁ agent, CPP, and 5-MeO-MiPT. In embodiments, the combination comprises rilmenidine, a CB₁ agent, CPP, and AGH-192. In embodiments, the combination comprises rilmenidine, a CB₁ agent, CPP, and AGH-107. In embodiments, the combination comprises rilmenidine, a CB₁ agent, CPP, and AMT. In embodiments, the combination comprises rilmenidine, a CB₁ agent, CPP, and aripiprazole. In embodiments, the combination comprises rilmenidine, a CB₁ agent, CPP, and AS-19. In embodiments, the combination comprises rilmenidine, a CB₁ agent, CPP, and E-55888. In embodiments, the combination comprises rilmenidine, a CB₁ agent, CPP, and LSD. In embodiments, the combination comprises rilmenidine, a CB₁ agent, CPP, and LP-211. In embodiments, the combination comprises rilmenidine, a CB₁ agent, CPP, and LP-44.

In embodiments, the combination comprises rilmenidine, a CB₁ agent, NBOH-2C-CN, and 5-CT. In embodiments, the combination comprises rilmenidine, a CB₁ agent, NBOH-2C-CN, and 5-MeO-DALT. In embodiments, the combination comprises rilmenidine, a CB₁ agent, NBOH-2C-CN, and 5-MeO-DMT. In embodiments, the combination comprises rilmenidine, a CB₁ agent, NBOH-2C-CN, and 5-MeO-MiPT In embodiments, the combination comprises rilmenidine, a CB₁ agent, NBOH-2C-CN, and AGH-192. In embodiments, the combination comprises rilmenidine, a CB₁ agent, NBOH-2C-CN, and AGH-107. In embodiments, the combination comprises rilmenidine, a CB₁ agent, NBOH-2C-CN, and AMT. In embodiments, the combination comprises rilmenidine, a CB₁ agent, NBOH-2C-CN, and aripiprazole. In embodiments, the combination comprises rilmenidine, a CB₁ agent, NBOH-2C-CN, and AS-19. In embodiments, the combination comprises rilmenidine, a CB₁ agent, NBOH-2C-CN, and E-55888. In embodiments, the combination comprises rilmenidine, a CB₁ agent, NBOH-2C-CN, and LSD. In embodiments, the combination comprises rilmenidine, a CB₁ agent, NBOH-2C-CN, and LP-211. In embodiments, the combination comprises rilmenidine, a CB₁ agent, NBOH-2C-CN, and LP-44.

In embodiments, the combination comprises rilmenidine, a CB₁ agent, TCB-2, and 5-CT. In embodiments, the combination comprises rilmenidine, a CB₁ agent, TCB-2, and 5-MeO-DALT. In embodiments, the combination comprises rilmenidine, a CB₁ agent, TCB-2, and 5-MeO-DMT In embodiments, the combination comprises rilmenidine, a CB₁ agent, TCB-2, and 5-MeO-MiPT. In embodiments, the combination comprises rilmenidine, a CB₁ agent, TCB-2, and AGH-192. In embodiments, the combination comprises rilmenidine, a CB₁ agent, TCB-2, and AGH-107. In embodiments, the combination comprises rilmenidine, a CB₁ agent, TCB-2, and AMT. In embodiments, the combination comprises rilmenidine, a CB₁ agent, TCB-2, and aripiprazole. In embodiments, the combination comprises rilmenidine, a CB₁ agent, TCB-2, and AS-19. In embodiments, the combination comprises rilmenidine, a CB₁ agent, TCB-2, and E-55888. In embodiments, the combination comprises rilmenidine, a CB₁ agent, TCB-2, and LSD. In embodiments, the combination comprises rilmenidine, a CB₁ agent, TCB-2, and LP-211. In embodiments, the combination comprises rilmenidine, a CB₁ agent, TCB-2, and LP-44.

In embodiments, the combination comprises rilmenidine, a CB₁ agent, WAY 161503, and 5-CT. In embodiments, the combination comprises rilmenidine, a CB₁ agent, WAY 161503, and 5-MeO-DALT. In embodiments, the combination comprises rilmenidine, a CB₁ agent, WAY 161503, and 5-MeO-DMT. In embodiments, the combination comprises rilmenidine, a CB₁ agent, WAY 161503, and 5-MeO-MiPT In embodiments, the combination comprises rilmenidine, a CB₁ agent, WAY 161503, and AGH-192. In embodiments, the combination comprises rilmenidine, a CB₁ agent, WAY 161503, and AGH-107. In embodiments, the combination comprises rilmenidine, a CB₁ agent, WAY 161503, and AMT. In embodiments, the combination comprises rilmenidine, a CB₁ agent, WAY 161503, and aripiprazole. In embodiments, the combination comprises rilmenidine, a CB₁ agent, WAY 161503, and AS-19. In embodiments, the combination comprises rilmenidine, a CB₁ agent, WAY 161503, and E-55888. In embodiments, the combination comprises rilmenidine, a CB₁ agent, WAY 161503, and LSD. In embodiments, the combination comprises rilmenidine, a CB₁ agent, WAY 161503, and LP-211. In embodiments, the combination comprises rilmenidine, a CB₁ agent, WAY 161503, and LP-44.

In embodiments, the combination comprises tetryzoline, a CB₁ agent, 1-methylpsilocin, and 5-CT. In embodiments, the combination comprises tetryzoline, a CB₁ agent, 1-methylpsilocin, and 5-MeO-DALT. In embodiments, the combination comprises tetryzoline, a CB₁ agent, 1-methylpsilocin, and 5-MeO-DMT In embodiments, the combination comprises tetryzoline, a CB₁ agent, 1-methylpsilocin, and 5-MeO-MiPT. In embodiments, the combination comprises tetryzoline, a CB₁ agent, 1-methylpsilocin, and AGH-192. In embodiments, the combination comprises tetryzoline, a CB₁ agent, 1-methylpsilocin, and AGH-107. In embodiments, the combination comprises tetryzoline, a CB₁ agent, 1-methylpsilocin, and AMT. In embodiments, the combination comprises tetryzoline, a CB₁ agent, 1-methylpsilocin, and aripiprazole. In embodiments, the combination comprises tetryzoline, a CB₁ agent, 1-methylpsilocin, and AS-19. In embodiments, the combination comprises tetryzoline, a CB₁ agent, 1-methylpsilocin, and E-55888. In embodiments, the combination comprises tetryzoline, a CB₁ agent, 1-methylpsilocin, and LSD. In embodiments, the combination comprises tetryzoline, a CB₁ agent, 1-methylpsilocin, and LP-211. In embodiments, the combination comprises tetryzoline, a CB₁ agent, 1-methylpsilocin, and LP-44.

In embodiments, the combination comprises tetryzoline, a CB₁ agent, 2C-B-FLY, and 5-CT. In embodiments, the combination comprises tetryzoline, a CB₁ agent, 2C-B-FLY, and 5-MeO-DALT. In embodiments, the combination comprises tetryzoline, a CB₁ agent, 2C-B-FLY, and 5-MeO-DMT. In embodiments, the combination comprises tetryzoline, a CB₁ agent, 2C-B-FLY, and 5-MeO-MiPT In embodiments, the combination comprises tetryzoline, a CB₁ agent, 2C-B-FLY, and AGH-192. In embodiments, the combination comprises tetryzoline, a CB₁ agent, 2C-B-FLY, and AGH-107. In embodiments, the combination comprises tetryzoline, a CB₁ agent, 2C-B-FLY, and AMT. In embodiments, the combination comprises tetryzoline, a CB₁ agent, 2C-B-FLY, and aripiprazole. In embodiments, the combination comprises tetryzoline, a CB₁ agent, 2C-B-FLY, and AS-19. In embodiments, the combination comprises tetryzoline, a CB₁ agent, 2C-B-FLY, and E-55888. In embodiments, the combination comprises tetryzoline, a CB₁ agent, 2C-B-FLY, and LSD. In embodiments, the combination comprises tetryzoline, a CB₁ agent, 2C-B-FLY, and LP-211. In embodiments, the combination comprises tetryzoline, a CB₁ agent, 2C-B-FLY, and LP-44.

In embodiments, the combination comprises tetryzoline, a CB₁ agent, 5-MeO-AMT, and 5-CT. In embodiments, the combination comprises tetryzoline, a CB₁ agent, 5-MeO-AMT, and 5-MeO-DALT. In embodiments, the combination comprises tetryzoline, a CB₁ agent, 5-MeO-AMT, and 5-MeO-DMT In embodiments, the combination comprises tetryzoline, a CB₁ agent, 5-MeO-AMT, and 5-MeO-MiPT. In embodiments, the combination comprises tetryzoline, a CB₁ agent, 5-MeO-AMT, and AGH-192. In embodiments, the combination comprises tetryzoline, a CB₁ agent, 5-MeO-AMT, and AGH-107. In embodiments, the combination comprises tetryzoline, a CB₁ agent, 5-MeO-AMT, and AMT. In embodiments, the combination comprises tetryzoline, a CB₁ agent, 5-MeO-AMT, and aripiprazole. In embodiments, the combination comprises tetryzoline, a CB₁ agent, 5-MeO-AMT, and AS-19. In embodiments, the combination comprises tetryzoline, a CB₁ agent, 5-MeO-AMT, and E-55888. In embodiments, the combination comprises tetryzoline, a CB₁ agent, 5-MeO-AMT, and LSD. In embodiments, the combination comprises tetryzoline, a CB₁ agent, 5-MeO-AMT, and LP-211. In embodiments, the combination comprises tetryzoline, a CB₁ agent, 5-MeO-AMT, and LP-44.

In embodiments, the combination comprises tetryzoline, a CB₁ agent, AL-37350A, and 5-CT. In embodiments, the combination comprises tetryzoline, a CB₁ agent, AL-37350A, and 5-MeO-DALT. In embodiments, the combination comprises tetryzoline, a CB₁ agent, AL-37350A, and 5-MeO-DMT. In embodiments, the combination comprises tetryzoline, a CB₁ agent, AL-37350A, and 5-MeO-MiPT In embodiments, the combination comprises tetryzoline, a CB₁ agent, AL-37350A, and AGH-192. In embodiments, the combination comprises tetryzoline, a CB₁ agent, AL-37350A, and AGH-107. In embodiments, the combination comprises tetryzoline, a CB₁ agent, AL-37350A, and AMT. In embodiments, the combination comprises tetryzoline, a CB₁ agent, AL-37350A, and aripiprazole. In embodiments, the combination comprises tetryzoline, a CB₁ agent, AL-37350A, and AS-19. In embodiments, the combination comprises tetryzoline, a CB₁ agent, AL-37350A, and E-55888. In embodiments, the combination comprises tetryzoline, a CB₁ agent, AL-37350A, and LSD. In embodiments, the combination comprises tetryzoline, a CB₁ agent, AL-37350A, and LP-211. In embodiments, the combination comprises tetryzoline, a CB₁ agent, AL-37350A, and LP-44.

In embodiments, the combination comprises tetryzoline, a CB₁ agent, CP 809101, and 5-CT. In embodiments, the combination comprises tetryzoline, a CB₁ agent, CP 809101, and 5-MeO-DALT. In embodiments, the combination comprises tetryzoline, a CB₁ agent, CP 809101, and 5-MeO-DMT. In embodiments, the combination comprises tetryzoline, a CB₁ agent, CP 809101, and 5-MeO-MiPT In embodiments, the combination comprises tetryzoline, a CB₁ agent, CP 809101, and AGH-192. In embodiments, the combination comprises tetryzoline, a CB₁ agent, CP 809101, and AGH-107. In embodiments, the combination comprises tetryzoline, a CB₁ agent, CP 809101, and AMT. In embodiments, the combination comprises tetryzoline, a CB₁ agent, CP 809101, and aripiprazole. In embodiments, the combination comprises tetryzoline, a CB₁ agent, CP 809101, and AS-19. In embodiments, the combination comprises tetryzoline, a CB₁ agent, CP 809101, and E-55888. In embodiments, the combination comprises tetryzoline, a CB₁ agent, CP 809101, and LSD. In embodiments, the combination comprises tetryzoline, a CB₁ agent, CP 809101, and LP-211. In embodiments, the combination comprises tetryzoline, a CB₁ agent, CP 809101, and LP-44.

In embodiments, the combination comprises tetryzoline, a CB₁ agent, DALT, and 5-CT. In embodiments, the combination comprises tetryzoline, a CB₁ agent, DALT, and 5-MeO-DALT. In embodiments, the combination comprises tetryzoline, a CB₁ agent, DALT, and 5-MeO-DMT. In embodiments, the combination comprises tetryzoline, a CB₁ agent, DALT, and 5-MeO-MiPT In embodiments, the combination comprises tetryzoline, a CB₁ agent, DALT, and AGH-192. In embodiments, the combination comprises tetryzoline, a CB₁ agent, DALT, and AGH-107. In embodiments, the combination comprises tetryzoline, a CB₁ agent, DALT, and AMT. In embodiments, the combination comprises tetryzoline, a CB₁ agent, DALT, and aripiprazole. In embodiments, the combination comprises tetryzoline, a CB₁ agent, DALT, and AS-19. In embodiments, the combination comprises tetryzoline, a CB₁ agent, DALT, and E-55888. In embodiments, the combination comprises tetryzoline, a CB₁ agent, DALT, and LSD. In embodiments, the combination comprises tetryzoline, a CB₁ agent, DALT, and LP-211. In embodiments, the combination comprises tetryzoline, a CB₁ agent, DALT, and LP-44.

In embodiments, the combination comprises tetryzoline, a CB₁ agent, DOB, and 5-CT. In embodiments, the combination comprises tetryzoline, a CB₁ agent, DOB, and 5-MeO-DALT. In embodiments, the combination comprises tetryzoline, a CB₁ agent, DOB, and 5-MeO-DMT. In embodiments, the combination comprises tetryzoline, a CB₁ agent, DOB, and 5-MeO-MiPT In embodiments, the combination comprises tetryzoline, a CB₁ agent, DOB, and AGH-192. In embodiments, the combination comprises tetryzoline, a CB₁ agent, DOB, and AGH-107. In embodiments, the combination comprises tetryzoline, a CB₁ agent, DOB, and AMT. In embodiments, the combination comprises tetryzoline, a CB₁ agent, DOB, and aripiprazole. In embodiments, the combination comprises tetryzoline, a CB₁ agent, DOB, and AS-19. In embodiments, the combination comprises tetryzoline, a CB₁ agent, DOB, and E-55888. In embodiments, the combination comprises tetryzoline, a CB₁ agent, DOB, and LSD. In embodiments, the combination comprises tetryzoline, a CB₁ agent, DOB, and LP-211. In embodiments, the combination comprises tetryzoline, a CB₁ agent, DOB, and LP-44.

In embodiments, the combination comprises tetryzoline, a CB₁ agent, DOET, and 5-CT. In embodiments, the combination comprises tetryzoline, a CB₁ agent, DOET, and 5-MeO-DALT. In embodiments, the combination comprises tetryzoline, a CB₁ agent, DOET, and 5-MeO-DMT In embodiments, the combination comprises tetryzoline, a CB₁ agent, DOET, and 5-MeO-MiPT. In embodiments, the combination comprises tetryzoline, a CB₁ agent, DOET, and AGH-192. In embodiments, the combination comprises tetryzoline, a CB₁ agent, DOET, and AGH-107. In embodiments, the combination comprises tetryzoline, a CB₁ agent, DOET, and AMT. In embodiments, the combination comprises tetryzoline, a CB₁ agent, DOET, and aripiprazole. In embodiments, the combination comprises tetryzoline, a CB₁ agent, DOET, and AS-19. In embodiments, the combination comprises tetryzoline, a CB₁ agent, DOET, and E-55888. In embodiments, the combination comprises tetryzoline, a CB₁ agent, DOET, and LSD. In embodiments, the combination comprises tetryzoline, a CB₁ agent, DOET, and LP-211. In embodiments, the combination comprises tetryzoline, a CB₁ agent, DOET, and LP-44.

In embodiments, the combination comprises tetryzoline, a CB₁ agent, DOHx, and 5-CT. In embodiments, the combination comprises tetryzoline, a CB₁ agent, DOHx, and 5-MeO-DALT. In embodiments, the combination comprises tetryzoline, a CB₁ agent, DOHx, and 5-MeO-DMT. In embodiments, the combination comprises tetryzoline, a CB₁ agent, DOHx, and 5-MeO-MiPT In embodiments, the combination comprises tetryzoline, a CB₁ agent, DOHx, and AGH-192. In embodiments, the combination comprises tetryzoline, a CB₁ agent, DOHx, and AGH-107. In embodiments, the combination comprises tetryzoline, a CB₁ agent, DOHx, and AMT. In embodiments, the combination comprises tetryzoline, a CB₁ agent, DOHx, and aripiprazole. In embodiments, the combination comprises tetryzoline, a CB₁ agent, DOHx, and AS-19. In embodiments, the combination comprises tetryzoline, a CB₁ agent, DOHx, and E-55888. In embodiments, the combination comprises tetryzoline, a CB₁ agent, DOHx, and LSD. In embodiments, the combination comprises tetryzoline, a CB₁ agent, DOHx, and LP-211. In embodiments, the combination comprises tetryzoline, a CB₁ agent, DOHx, and LP-44.

In embodiments, the combination comprises tetryzoline, a CB₁ agent, MEM, and 5-CT. In embodiments, the combination comprises tetryzoline, a CB₁ agent, MEM, and 5-MeO-DALT. In embodiments, the combination comprises tetryzoline, a CB₁ agent, MEM, and 5-MeO-DMT In embodiments, the combination comprises tetryzoline, a CB₁ agent, MEM, and 5-MeO-MiPT. In embodiments, the combination comprises tetryzoline, a CB₁ agent, MEM, and AGH-192. In embodiments, the combination comprises tetryzoline, a CB₁ agent, MEM, and AGH-107. In embodiments, the combination comprises tetryzoline, a CB₁ agent, MEM, and AMT. In embodiments, the combination comprises tetryzoline, a CB₁ agent, MEM, and aripiprazole. In embodiments, the combination comprises tetryzoline, a CB₁ agent, MEM, and AS-19. In embodiments, the combination comprises tetryzoline, a CB₁ agent, MEM, and E-55888. In embodiments, the combination comprises tetryzoline, a CB₁ agent, MEM, and LSD. In embodiments, the combination comprises tetryzoline, a CB₁ agent, MEM, and LP-211. In embodiments, the combination comprises tetryzoline, a CB₁ agent, MEM, and LP-44.

In embodiments, the combination comprises tetryzoline, a CB₁ agent, CPP, and 5-CT. In embodiments, the combination comprises tetryzoline, a CB₁ agent, CPP, and 5-MeO-DALT. In embodiments, the combination comprises tetryzoline, a CB₁ agent, CPP, and 5-MeO-DMT In embodiments, the combination comprises tetryzoline, a CB₁ agent, CPP, and 5-MeO-MiPT. In embodiments, the combination comprises tetryzoline, a CB₁ agent, CPP, and AGH-192. In embodiments, the combination comprises tetryzoline, a CB₁ agent, CPP, and AGH-107. In embodiments, the combination comprises tetryzoline, a CB₁ agent, CPP, and AMT. In embodiments, the combination comprises tetryzoline, a CB₁ agent, CPP, and aripiprazole. In embodiments, the combination comprises tetryzoline, a CB₁ agent, CPP, and AS-19. In embodiments, the combination comprises tetryzoline, a CB₁ agent, CPP, and E-55888. In embodiments, the combination comprises tetryzoline, a CB₁ agent, CPP, and LSD. In embodiments, the combination comprises tetryzoline, a CB₁ agent, CPP, and LP-211. In embodiments, the combination comprises tetryzoline, a CB₁ agent, CPP, and LP-44.

In embodiments, the combination comprises tetryzoline, a CB₁ agent, NBOH-2C-CN, and 5-CT. In embodiments, the combination comprises tetryzoline, a CB₁ agent, NBOH-2C-CN, and 5-MeO-DALT. In embodiments, the combination comprises tetryzoline, a CB₁ agent, NBOH-2C-CN, and 5-MeO-DMT. In embodiments, the combination comprises tetryzoline, a CB₁ agent, NBOH-2C-CN, and 5-MeO-MiPT. In embodiments, the combination comprises tetryzoline, a CB₁ agent, NBOH-2C-CN, and AGH-192. In embodiments, the combination comprises tetryzoline, a CB₁ agent, NBOH-2C-CN, and AGH-107. In embodiments, the combination comprises tetryzoline, a CB₁ agent, NBOH-2C-CN, and AMT. In embodiments, the combination comprises tetryzoline, a CB₁ agent, NBOH-2C-CN, and aripiprazole. In embodiments, the combination comprises tetryzoline, a CB₁ agent, NBOH-2C-CN, and AS-19. In embodiments, the combination comprises tetryzoline, a CB₁ agent, NBOH-2C-CN, and E-55888. In embodiments, the combination comprises tetryzoline, a CB₁ agent, NBOH-2C-CN, and LSD. In embodiments, the combination comprises tetryzoline, a CB₁ agent, NBOH-2C-CN, and LP-211. In embodiments, the combination comprises tetryzoline, a CB₁ agent, NBOH-2C-CN, and LP-44.

In embodiments, the combination comprises tetryzoline, a CB₁ agent, TCB-2, and 5-CT. In embodiments, the combination comprises tetryzoline, a CB₁ agent, TCB-2, and 5-MeO-DALT. In embodiments, the combination comprises tetryzoline, a CB₁ agent, TCB-2, and 5-MeO-DMT In embodiments, the combination comprises tetryzoline, a CB₁ agent, TCB-2, and 5-MeO-MiPT. In embodiments, the combination comprises tetryzoline, a CB₁ agent, TCB-2, and AGH-192. In embodiments, the combination comprises tetryzoline, a CB₁ agent, TCB-2, and AGH-107. In embodiments, the combination comprises tetryzoline, a CB₁ agent, TCB-2, and AMT. In embodiments, the combination comprises tetryzoline, a CB₁ agent, TCB-2, and aripiprazole. In embodiments, the combination comprises tetryzoline, a CB₁ agent, TCB-2, and AS-19. In embodiments, the combination comprises tetryzoline, a CB₁ agent, TCB-2, and E-55888. In embodiments, the combination comprises tetryzoline, a CB₁ agent, TCB-2, and LSD. In embodiments, the combination comprises tetryzoline, a CB₁ agent, TCB-2, and LP-211. In embodiments, the combination comprises tetryzoline, a CB₁ agent, TCB-2, and LP-44.

In embodiments, the combination comprises tetryzoline, a CB₁ agent, WAY 161503, and 5-CT. In embodiments, the combination comprises tetryzoline, a CB₁ agent, WAY 161503, and 5-MeO-DALT. In embodiments, the combination comprises tetryzoline, a CB₁ agent, WAY 161503, and 5-MeO-DMT. In embodiments, the combination comprises tetryzoline, a CB₁ agent, WAY 161503, and 5-MeO-MiPT In embodiments, the combination comprises tetryzoline, a CB₁ agent, WAY 161503, and AGH-192. In embodiments, the combination comprises tetryzoline, a CB₁ agent, WAY 161503, and AGH-107. In embodiments, the combination comprises tetryzoline, a CB₁ agent, WAY 161503, and AMT. In embodiments, the combination comprises tetryzoline, a CB₁ agent, WAY 161503, and aripiprazole. In embodiments, the combination comprises tetryzoline, a CB₁ agent, WAY 161503, and AS-19. In embodiments, the combination comprises tetryzoline, a CB₁ agent, WAY 161503, and E-55888. In embodiments, the combination comprises tetryzoline, a CB₁ agent, WAY 161503, and LSD. In embodiments, the combination comprises tetryzoline, a CB₁ agent, WAY 161503, and LP-211. In embodiments, the combination comprises tetryzoline, a CB₁ agent, WAY 161503, and LP-44.

In embodiments, the combination comprises tolonidine, a CB₁ agent, 1-methylpsilocin, and 5-CT. In embodiments, the combination comprises tolonidine, a CB₁ agent, 1-methylpsilocin, and 5-MeO-DALT. In embodiments, the combination comprises tolonidine, a CB₁ agent, 1-methylpsilocin, and 5-MeO-DMT In embodiments, the combination comprises tolonidine, a CB₁ agent, 1-methylpsilocin, and 5-MeO-MiPT In embodiments, the combination comprises tolonidine, a CB₁ agent, 1-methylpsilocin, and AGH-192. In embodiments, the combination comprises tolonidine, a CB₁ agent, 1-methylpsilocin, and AGH-107. In embodiments, the combination comprises tolonidine, a CB₁ agent, 1-methylpsilocin, and AMT. In embodiments, the combination comprises tolonidine, a CB₁ agent, 1-methylpsilocin, and aripiprazole. In embodiments, the combination comprises tolonidine, a CB₁ agent, 1-methylpsilocin, and AS-19. In embodiments, the combination comprises tolonidine, a CB₁ agent, 1-methylpsilocin, and E-55888. In embodiments, the combination comprises tolonidine, a CB₁ agent, 1-methylpsilocin, and LSD. In embodiments, the combination comprises tolonidine, a CB₁ agent, 1-methylpsilocin, and LP-211. In embodiments, the combination comprises tolonidine, a CB₁ agent, 1-methyl psilocin, and LP-44.

In embodiments, the combination comprises tolonidine, a CB₁ agent, 2C-B-FLY, and 5-CT. In embodiments, the combination comprises tolonidine, a CB₁ agent, 2C-B-FLY, and 5-MeO-DALT. In embodiments, the combination comprises tolonidine, a CB₁ agent, 2C-B-FLY, and 5-MeO-DMT. In embodiments, the combination comprises tolonidine, a CB₁ agent, 2C-B-FLY, and 5-MeO-MiPT In embodiments, the combination comprises tolonidine, a CB₁ agent, 2C-B-FLY, and AGH-192. In embodiments, the combination comprises tolonidine, a CB₁ agent, 2C-B-FLY, and AGH-107. In embodiments, the combination comprises tolonidine, a CB₁ agent, 2C-B-FLY, and AMT. In embodiments, the combination comprises tolonidine, a CB₁ agent, 2C-B-FLY, and aripiprazole. In embodiments, the combination comprises tolonidine, a CB₁ agent, 2C-B-FLY, and AS-19. In embodiments, the combination comprises tolonidine, a CB₁ agent, 2C-B-FLY, and E-55888. In embodiments, the combination comprises tolonidine, a CB₁ agent, 2C-B-FLY, and LSD. In embodiments, the combination comprises tolonidine, a CB₁ agent, 2C-B-FLY, and LP-211. In embodiments, the combination comprises tolonidine, a CB₁ agent, 2C-B-FLY, and LP-44.

In embodiments, the combination comprises tolonidine, a CB₁ agent, 5-MeO-AMT, and 5-CT. In embodiments, the combination comprises tolonidine, a CB₁ agent, 5-MeO-AMT, and 5-MeO-DALT. In embodiments, the combination comprises tolonidine, a CB₁ agent, 5-MeO-AMT, and 5-MeO-DMT. In embodiments, the combination comprises tolonidine, a CB₁ agent, 5-MeO-AMT, and 5-MeO-MiPT. In embodiments, the combination comprises tolonidine, a CB₁ agent, 5-MeO-AMT, and AGH-192. In embodiments, the combination comprises tolonidine, a CB₁ agent, 5-MeO-AMT, and AGH-107. In embodiments, the combination comprises tolonidine, a CB₁ agent, 5-MeO-AMT, and AMT. In embodiments, the combination comprises tolonidine, a CB₁ agent, 5-MeO-AMT, and aripiprazole. In embodiments, the combination comprises tolonidine, a CB₁ agent, 5-MeO-AMT, and AS-19. In embodiments, the combination comprises tolonidine, a CB₁ agent, 5-MeO-AMT, and E-55888. In embodiments, the combination comprises tolonidine, a CB₁ agent, 5-MeO-AMT, and LSD. In embodiments, the combination comprises tolonidine, a CB₁ agent, 5-MeO-AMT, and LP-211. In embodiments, the combination comprises tolonidine, a CB₁ agent, 5-MeO-AMT, and LP-44.

In embodiments, the combination comprises tolonidine, a CB₁ agent, AL-37350A, and 5-CT. In embodiments, the combination comprises tolonidine, a CB₁ agent, AL-37350A, and 5-MeO-DALT. In embodiments, the combination comprises tolonidine, a CB₁ agent, AL-37350A, and 5-MeO-DMT. In embodiments, the combination comprises tolonidine, a CB₁ agent, AL-37350A, and 5-MeO-MiPT In embodiments, the combination comprises tolonidine, a CB₁ agent, AL-37350A, and AGH-192. In embodiments, the combination comprises tolonidine, a CB₁ agent, AL-37350A, and AGH-107. In embodiments, the combination comprises tolonidine, a CB₁ agent, AL-37350A, and AMT. In embodiments, the combination comprises tolonidine, a CB₁ agent, AL-37350A, and aripiprazole. In embodiments, the combination comprises tolonidine, a CB₁ agent, AL-37350A, and AS-19. In embodiments, the combination comprises tolonidine, a CB₁ agent, AL-37350A, and E-55888. In embodiments, the combination comprises tolonidine, a CB₁ agent, AL-37350A, and LSD. In embodiments, the combination comprises tolonidine, a CB₁ agent, AL-37350A, and LP-211. In embodiments, the combination comprises tolonidine, a CB₁ agent, AL-37350A, and LP-44.

In embodiments, the combination comprises tolonidine, a CB₁ agent, CP 809101, and 5-CT. In embodiments, the combination comprises tolonidine, a CB₁ agent, CP 809101, and 5-MeO-DALT. In embodiments, the combination comprises tolonidine, a CB₁ agent, CP 809101, and 5-MeO-DMT. In embodiments, the combination comprises tolonidine, a CB₁ agent, CP 809101, and 5-MeO-MiPT In embodiments, the combination comprises tolonidine, a CB₁ agent, CP 809101, and AGH-192. In embodiments, the combination comprises tolonidine, a CB₁ agent, CP 809101, and AGH-107. In embodiments, the combination comprises tolonidine, a CB₁ agent, CP 809101, and AMT. In embodiments, the combination comprises tolonidine, a CB₁ agent, CP 809101, and aripiprazole. In embodiments, the combination comprises tolonidine, a CB₁ agent, CP 809101, and AS-19. In embodiments, the combination comprises tolonidine, a CB₁ agent, CP 809101, and E-55888. In embodiments, the combination comprises tolonidine, a CB₁ agent, CP 809101, and LSD. In embodiments, the combination comprises tolonidine, a CB₁ agent, CP 809101, and LP-211. In embodiments, the combination comprises tolonidine, a CB₁ agent, CP 809101, and LP-44.

In embodiments, the combination comprises tolonidine, a CB₁ agent, DALT, and 5-CT. In embodiments, the combination comprises tolonidine, a CB₁ agent, DALT, and 5-MeO-DALT. In embodiments, the combination comprises tolonidine, a CB₁ agent, DALT, and 5-MeO-DMT. In embodiments, the combination comprises tolonidine, a CB₁ agent, DALT, and 5-MeO-MiPT In embodiments, the combination comprises tolonidine, a CB₁ agent, DALT, and AGH-192. In embodiments, the combination comprises tolonidine, a CB₁ agent, DALT, and AGH-107. In embodiments, the combination comprises tolonidine, a CB₁ agent, DALT, and AMT. In embodiments, the combination comprises tolonidine, a CB₁ agent, DALT, and aripiprazole. In embodiments, the combination comprises tolonidine, a CB₁ agent, DALT, and AS-19. In embodiments, the combination comprises tolonidine, a CB₁ agent, DALT, and E-55888. In embodiments, the combination comprises tolonidine, a CB₁ agent, DALT, and LSD. In embodiments, the combination comprises tolonidine, a CB₁ agent, DALT, and LP-211. In embodiments, the combination comprises tolonidine, a CB₁ agent, DALT, and LP-44.

In embodiments, the combination comprises tolonidine, a CB₁ agent, DOB, and 5-CT. In embodiments, the combination comprises tolonidine, a CB₁ agent, DOB, and 5-MeO-DALT. In embodiments, the combination comprises tolonidine, a CB₁ agent, DOB, and 5-MeO-DMT. In embodiments, the combination comprises tolonidine, a CB₁ agent, DOB, and 5-MeO-MiPT In embodiments, the combination comprises tolonidine, a CB₁ agent, DOB, and AGH-192. In embodiments, the combination comprises tolonidine, a CB₁ agent, DOB, and AGH-107. In embodiments, the combination comprises tolonidine, a CB₁ agent, DOB, and AMT. In embodiments, the combination comprises tolonidine, a CB₁ agent, DOB, and aripiprazole. In embodiments, the combination comprises tolonidine, a CB₁ agent, DOB, and AS-19. In embodiments, the combination comprises tolonidine, a CB₁ agent, DOB, and E-55888. In embodiments, the combination comprises tolonidine, a CB₁ agent, DOB, and LSD. In embodiments, the combination comprises tolonidine, a CB₁ agent, DOB, and LP-211. In embodiments, the combination comprises tolonidine, a CB₁ agent, DOB, and LP-44.

In embodiments, the combination comprises tolonidine, a CB₁ agent, DOET, and 5-CT. In embodiments, the combination comprises tolonidine, a CB₁ agent, DOET, and 5-MeO-DALT. In embodiments, the combination comprises tolonidine, a CB₁ agent, DOET, and 5-MeO-DMT In embodiments, the combination comprises tolonidine, a CB₁ agent, DOET, and 5-MeO-MiPT In embodiments, the combination comprises tolonidine, a CB₁ agent, DOET, and AGH-192. In embodiments, the combination comprises tolonidine, a CB₁ agent, DOET, and AGH-107. In embodiments, the combination comprises tolonidine, a CB₁ agent, DOET, and AMT. In embodiments, the combination comprises tolonidine, a CB₁ agent, DOET, and aripiprazole. In embodiments, the combination comprises tolonidine, a CB₁ agent, DOET, and AS-19. In embodiments, the combination comprises tolonidine, a CB₁ agent, DOET, and E-55888. In embodiments, the combination comprises tolonidine, a CB₁ agent, DOET, and LSD. In embodiments, the combination comprises tolonidine, a CB₁ agent, DOET, and LP-211. In embodiments, the combination comprises tolonidine, a CB₁ agent, DOET, and LP-44.

In embodiments, the combination comprises tolonidine, a CB₁ agent, DOHx, and 5-CT. In embodiments, the combination comprises tolonidine, a CB₁ agent, DOHx, and 5-MeO-DALT. In embodiments, the combination comprises tolonidine, a CB₁ agent, DOHx, and 5-MeO-DMT. In embodiments, the combination comprises tolonidine, a CB₁ agent, DOHx, and 5-MeO-MiPT In embodiments, the combination comprises tolonidine, a CB₁ agent, DOHx, and AGH-192. In embodiments, the combination comprises tolonidine, a CB₁ agent, DOHx, and AGH-107. In embodiments, the combination comprises tolonidine, a CB₁ agent, DOHx, and AMT. In embodiments, the combination comprises tolonidine, a CB₁ agent, DOHx, and aripiprazole. In embodiments, the combination comprises tolonidine, a CB₁ agent, DOHx, and AS-19. In embodiments, the combination comprises tolonidine, a CB₁ agent, DOHx, and E-55888. In embodiments, the combination comprises tolonidine, a CB₁ agent, DOHx, and LSD. In embodiments, the combination comprises tolonidine, a CB₁ agent, DOHx, and LP-211. In embodiments, the combination comprises tolonidine, a CB₁ agent, DOHx, and LP-44.

In embodiments, the combination comprises tolonidine, a CB₁ agent, MEM, and 5-CT. In embodiments, the combination comprises tolonidine, a CB₁ agent, MEM, and 5-MeO-DALT. In embodiments, the combination comprises tolonidine, a CB₁ agent, MEM, and 5-MeO-DMT In embodiments, the combination comprises tolonidine, a CB₁ agent, MEM, and 5-MeO-MiPT. In embodiments, the combination comprises tolonidine, a CB₁ agent, MEM, and AGH-192. In embodiments, the combination comprises tolonidine, a CB₁ agent, MEM, and AGH-107. In embodiments, the combination comprises tolonidine, a CB₁ agent, MEM, and AMT. In embodiments, the combination comprises tolonidine, a CB₁ agent, MEM, and aripiprazole. In embodiments, the combination comprises tolonidine, a CB₁ agent, MEM, and AS-19. In embodiments, the combination comprises tolonidine, a CB₁ agent, MEM, and E-55888. In embodiments, the combination comprises tolonidine, a CB₁ agent, MEM, and LSD. In embodiments, the combination comprises tolonidine, a CB₁ agent, MEM, and LP-211. In embodiments, the combination comprises tolonidine, a CB₁ agent, MEM, and LP-44.

In embodiments, the combination comprises tolonidine, a CB₁ agent, CPP, and 5-CT. In embodiments, the combination comprises tolonidine, a CB₁ agent, CPP, and 5-MeO-DALT. In embodiments, the combination comprises tolonidine, a CB₁ agent, CPP, and 5-MeO-DMT In embodiments, the combination comprises tolonidine, a CB₁ agent, CPP, and 5-MeO-MiPT In embodiments, the combination comprises tolonidine, a CB₁ agent, CPP, and AGH-192. In embodiments, the combination comprises tolonidine, a CB₁ agent, CPP, and AGH-107. In embodiments, the combination comprises tolonidine, a CB₁ agent, CPP, and AMT. In embodiments, the combination comprises tolonidine, a CB₁ agent, CPP, and aripiprazole. In embodiments, the combination comprises tolonidine, a CB₁ agent, CPP, and AS-19. In embodiments, the combination comprises tolonidine, a CB₁ agent, CPP, and E-55888. In embodiments, the combination comprises tolonidine, a CB₁ agent, CPP, and LSD. In embodiments, the combination comprises tolonidine, a CB₁ agent, CPP, and LP-211. In embodiments, the combination comprises tolonidine, a CB₁ agent, CPP, and LP-44.

In embodiments, the combination comprises tolonidine, a CB₁ agent, NBOH-2C-CN, and 5-CT. In embodiments, the combination comprises tolonidine, a CB₁ agent, NBOH-2C-CN, and 5-MeO-DALT. In embodiments, the combination comprises tolonidine, a CB₁ agent, NBOH-2C-CN, and 5-MeO-DMT. In embodiments, the combination comprises tolonidine, a CB₁ agent, NBOH-2C-CN, and 5-MeO-MiPT. In embodiments, the combination comprises tolonidine, a CB₁ agent, NBOH-2C-CN, and AGH-192. In embodiments, the combination comprises tolonidine, a CB₁ agent, NBOH-2C-CN, and AGH-107. In embodiments, the combination comprises tolonidine, a CB₁ agent, NBOH-2C-CN, and AMT. In embodiments, the combination comprises tolonidine, a CB₁ agent, NBOH-2C-CN, and aripiprazole. In embodiments, the combination comprises tolonidine, a CB₁ agent, NBOH-2C-CN, and AS-19. In embodiments, the combination comprises tolonidine, a CB₁ agent, NBOH-2C-CN, and E-55888. In embodiments, the combination comprises tolonidine, a CB₁ agent, NBOH-2C-CN, and LSD. In embodiments, the combination comprises tolonidine, a CB₁ agent, NBOH-2C-CN, and LP-211. In embodiments, the combination comprises tolonidine, a CB₁ agent, NBOH-2C-CN, and LP-44.

In embodiments, the combination comprises tolonidine, a CB₁ agent, TCB-2, and 5-CT. In embodiments, the combination comprises tolonidine, a CB₁ agent, TCB-2, and 5-MeO-DALT. In embodiments, the combination comprises tolonidine, a CB₁ agent, TCB-2, and 5-MeO-DMT In embodiments, the combination comprises tolonidine, a CB₁ agent, TCB-2, and 5-MeO-MiPT. In embodiments, the combination comprises tolonidine, a CB₁ agent, TCB-2, and AGH-192. In embodiments, the combination comprises tolonidine, a CB₁ agent, TCB-2, and AGH-107. In embodiments, the combination comprises tolonidine, a CB₁ agent, TCB-2, and AMT. In embodiments, the combination comprises tolonidine, a CB₁ agent, TCB-2, and aripiprazole. In embodiments, the combination comprises tolonidine, a CB₁ agent, TCB-2, and AS-19. In embodiments, the combination comprises tolonidine, a CB₁ agent, TCB-2, and E-55888. In embodiments, the combination comprises tolonidine, a CB₁ agent, TCB-2, and LSD. In embodiments, the combination comprises tolonidine, a CB₁ agent, TCB-2, and LP-211. In embodiments, the combination comprises tolonidine, a CB₁ agent, TCB-2, and LP-44.

In embodiments, the combination comprises tolonidine, a CB₁ agent, WAY 161503, and 5-CT. In embodiments, the combination comprises tolonidine, a CB₁ agent, WAY 161503, and 5-MeO-DALT In embodiments, the combination comprises tolonidine, a CB₁ agent, WAY 161503, and 5-MeO-DMT. In embodiments, the combination comprises tolonidine, a CB₁ agent, WAY 161503, and 5-MeO-MiPT In embodiments, the combination comprises tolonidine, a CB₁ agent, WAY 161503, and AGH-192. In embodiments, the combination comprises tolonidine, a CB₁ agent, WAY 161503, and AGH-107. In embodiments, the combination comprises tolonidine, a CB₁ agent, WAY 161503, and AMT. In embodiments, the combination comprises tolonidine, a CB₁ agent, WAY 161503, and aripiprazole. In embodiments, the combination comprises tolonidine, a CB₁ agent, WAY 161503, and AS-19. In embodiments, the combination comprises tolonidine, a CB₁ agent, WAY 161503, and E-55888. In embodiments, the combination comprises tolonidine, a CB₁ agent, WAY 161503, and LSD. In embodiments, the combination comprises tolonidine, a CB₁ agent, WAY 161503, and LP-211. In embodiments, the combination comprises tolonidine, a CB₁ agent, WAY 161503, and LP-44.

In embodiments, the combination comprises tizanidine, a CB₁ agent, 1-methylpsilocin, and 5-CT. In embodiments, the combination comprises tizanidine, a CB₁ agent, 1-methylpsilocin, and 5-MeO-DALT. In embodiments, the combination comprises tizanidine, a CB₁ agent, 1-methylpsilocin, and 5-MeO-DMT In embodiments, the combination comprises tizanidine, a CB₁ agent, 1-methylpsilocin, and 5-MeO-MiPT. In embodiments, the combination comprises tizanidine, a CB₁ agent, 1-methylpsilocin, and AGH-192. In embodiments, the combination comprises tizanidine, a CB₁ agent, 1-methylpsilocin, and AGH-107. In embodiments, the combination comprises tizanidine, a CB₁ agent, 1-methylpsilocin, and AMT. In embodiments, the combination comprises tizanidine, a CB₁ agent, 1-methylpsilocin, and aripiprazole. In embodiments, the combination comprises tizanidine, a CB₁ agent, 1-methylpsilocin, and AS-19. In embodiments, the combination comprises tizanidine, a CB₁ agent, 1-methylpsilocin, and E-55888. In embodiments, the combination comprises tizanidine, a CB₁ agent, 1-methylpsilocin, and LSD. In embodiments, the combination comprises tizanidine, a CB₁ agent, 1-methylpsilocin, and LP-211. In embodiments, the combination comprises tizanidine, a CB₁ agent, 1-methyl psilocin, and LP-44.

In embodiments, the combination comprises tizanidine, a CB₁ agent, 2C-B-FLY, and 5-CT. In embodiments, the combination comprises tizanidine, a CB₁ agent, 2C-B-FLY, and 5-MeO-DALT. In embodiments, the combination comprises tizanidine, a CB₁ agent, 2C-B-FLY, and 5-MeO-DMT. In embodiments, the combination comprises tizanidine, a CB₁ agent, 2C-B-FLY, and 5-MeO-MiPT In embodiments, the combination comprises tizanidine, a CB₁ agent, 2C-B-FLY, and AGH-192. In embodiments, the combination comprises tizanidine, a CB₁ agent, 2C-B-FLY, and AGH-107. In embodiments, the combination comprises tizanidine, a CB₁ agent, 2C-B-FLY, and AMT. In embodiments, the combination comprises tizanidine, a CB₁ agent, 2C-B-FLY, and aripiprazole. In embodiments, the combination comprises tizanidine, a CB₁ agent, 2C-B-FLY, and AS-19. In embodiments, the combination comprises tizanidine, a CB₁ agent, 2C-B-FLY, and E-55888. In embodiments, the combination comprises tizanidine, a CB₁ agent, 2C-B-FLY, and LSD. In embodiments, the combination comprises tizanidine, a CB₁ agent, 2C-B-FLY, and LP-211. In embodiments, the combination comprises tizanidine, a CB₁ agent, 2C-B-FLY, and LP-44.

In embodiments, the combination comprises tizanidine, a CB₁ agent, 5-MeO-AMT, and 5-CT. In embodiments, the combination comprises tizanidine, a CB₁ agent, 5-MeO-AMT, and 5-MeO-DALT. In embodiments, the combination comprises tizanidine, a CB₁ agent, 5-MeO-AMT, and 5-MeO-DMT. In embodiments, the combination comprises tizanidine, a CB₁ agent, 5-MeO-AMT, and 5-MeO-MiPT In embodiments, the combination comprises tizanidine, a CB₁ agent, 5-MeO-AMT, and AGH-192. In embodiments, the combination comprises tizanidine, a CB₁ agent, 5-MeO-AMT, and AGH-107. In embodiments, the combination comprises tizanidine, a CB₁ agent, 5-MeO-AMT, and AMT. In embodiments, the combination comprises tizanidine, a CB₁ agent, 5-MeO-AMT, and aripiprazole. In embodiments, the combination comprises tizanidine, a CB₁ agent, 5-MeO-AMT, and AS-19. In embodiments, the combination comprises tizanidine, a CB₁ agent, 5-MeO-AMT, and E-55888. In embodiments, the combination comprises tizanidine, a CB₁ agent, 5-MeO-AMT, and LSD. In embodiments, the combination comprises tizanidine, a CB₁ agent, 5-MeO-AMT, and LP-211. In embodiments, the combination comprises tizanidine, a CB₁ agent, 5-MeO-AMT, and LP-44.

In embodiments, the combination comprises tizanidine, a CB₁ agent, AL-37350A, and 5-CT. In embodiments, the combination comprises tizanidine, a CB₁ agent, AL-37350A, and 5-MeO-DALT. In embodiments, the combination comprises tizanidine, a CB₁ agent, AL-37350A, and 5-MeO-DMT. In embodiments, the combination comprises tizanidine, a CB₁ agent, AL-37350A, and 5-MeO-MiPT In embodiments, the combination comprises tizanidine, a CB₁ agent, AL-37350A, and AGH-192. In embodiments, the combination comprises tizanidine, a CB₁ agent, AL-37350A, and AGH-107. In embodiments, the combination comprises tizanidine, a CB₁ agent, AL-37350A, and AMT. In embodiments, the combination comprises tizanidine, a CB₁ agent, AL-37350A, and aripiprazole. In embodiments, the combination comprises tizanidine, a CB₁ agent, AL-37350A, and AS-19. In embodiments, the combination comprises tizanidine, a CB₁ agent, AL-37350A, and E-55888. In embodiments, the combination comprises tizanidine, a CB₁ agent, AL-37350A, and LSD. In embodiments, the combination comprises tizanidine, a CB₁ agent, AL-37350A, and LP-211. In embodiments, the combination comprises tizanidine, a CB₁ agent, AL-37350A, and LP-44.

In embodiments, the combination comprises tizanidine, a CB₁ agent, CP 809101, and 5-CT. In embodiments, the combination comprises tizanidine, a CB₁ agent, CP 809101, and 5-MeO-DALT. In embodiments, the combination comprises tizanidine, a CB₁ agent, CP 809101, and 5-MeO-DMT. In embodiments, the combination comprises tizanidine, a CB₁ agent, CP 809101, and 5-MeO-MiPT In embodiments, the combination comprises tizanidine, a CB₁ agent, CP 809101, and AGH-192. In embodiments, the combination comprises tizanidine, a CB₁ agent, CP 809101, and AGH-107. In embodiments, the combination comprises tizanidine, a CB₁ agent, CP 809101, and AMT. In embodiments, the combination comprises tizanidine, a CB₁ agent, CP 809101, and aripiprazole. In embodiments, the combination comprises tizanidine, a CB₁ agent, CP 809101, and AS-19. In embodiments, the combination comprises tizanidine, a CB₁ agent, CP 809101, and E-55888. In embodiments, the combination comprises tizanidine, a CB₁ agent, CP 809101, and LSD. In embodiments, the combination comprises tizanidine, a CB₁ agent, CP 809101, and LP-211. In embodiments, the combination comprises tizanidine, a CB₁ agent, CP 809101, and LP-44.

In embodiments, the combination comprises tizanidine, a CB₁ agent, DALT, and 5-CT. In embodiments, the combination comprises tizanidine, a CB₁ agent, DALT, and 5-MeO-DALT. In embodiments, the combination comprises tizanidine, a CB₁ agent, DALT, and 5-MeO-DMT. In embodiments, the combination comprises tizanidine, a CB₁ agent, DALT, and 5-MeO-MiPT In embodiments, the combination comprises tizanidine, a CB₁ agent, DALT, and AGH-192. In embodiments, the combination comprises tizanidine, a CB₁ agent, DALT, and AGH-107. In embodiments, the combination comprises tizanidine, a CB₁ agent, DALT, and AMT. In embodiments, the combination comprises tizanidine, a CB₁ agent, DALT, and aripiprazole. In embodiments, the combination comprises tizanidine, a CB₁ agent, DALT, and AS-19. In embodiments, the combination comprises tizanidine, a CB₁ agent, DALT, and E-55888. In embodiments, the combination comprises tizanidine, a CB₁ agent, DALT, and LSD. In embodiments, the combination comprises tizanidine, a CB₁ agent, DALT, and LP-211. In embodiments, the combination comprises tizanidine, a CB₁ agent, DALT, and LP-44.

In embodiments, the combination comprises tizanidine, a CB₁ agent, DOB, and 5-CT. In embodiments, the combination comprises tizanidine, a CB₁ agent, DOB, and 5-MeO-DALT. In embodiments, the combination comprises tizanidine, a CB₁ agent, DOB, and 5-MeO-DMT In embodiments, the combination comprises tizanidine, a CB₁ agent, DOB, and 5-MeO-MiPT In embodiments, the combination comprises tizanidine, a CB₁ agent, DOB, and AGH-192. In embodiments, the combination comprises tizanidine, a CB₁ agent, DOB, and AGH-107. In embodiments, the combination comprises tizanidine, a CB₁ agent, DOB, and AMT. In embodiments, the combination comprises tizanidine, a CB₁ agent, DOB, and aripiprazole. In embodiments, the combination comprises tizanidine, a CB₁ agent, DOB, and AS-19. In embodiments, the combination comprises tizanidine, a CB₁ agent, DOB, and E-55888. In embodiments, the combination comprises tizanidine, a CB₁ agent, DOB, and LSD. In embodiments, the combination comprises tizanidine, a CB₁ agent, DOB, and LP-211. In embodiments, the combination comprises tizanidine, a CB₁ agent, DOB, and LP-44.

In embodiments, the combination comprises tizanidine, a CB₁ agent, DOET, and 5-CT. In embodiments, the combination comprises tizanidine, a CB₁ agent, DOET, and 5-MeO-DALT. In embodiments, the combination comprises tizanidine, a CB₁ agent, DOET, and 5-MeO-DMT In embodiments, the combination comprises tizanidine, a CB₁ agent, DOET, and 5-MeO-MiPT. In embodiments, the combination comprises tizanidine, a CB₁ agent, DOET, and AGH-192. In embodiments, the combination comprises tizanidine, a CB₁ agent, DOET, and AGH-107. In embodiments, the combination comprises tizanidine, a CB₁ agent, DOET, and AMT. In embodiments, the combination comprises tizanidine, a CB₁ agent, DOET, and aripiprazole. In embodiments, the combination comprises tizanidine, a CB₁ agent, DOET, and AS-19. In embodiments, the combination comprises tizanidine, a CB₁ agent, DOET, and E-55888. In embodiments, the combination comprises tizanidine, a CB₁ agent, DOET, and LSD. In embodiments, the combination comprises tizanidine, a CB₁ agent, DOET, and LP-211. In embodiments, the combination comprises tizanidine, a CB₁ agent, DOET, and LP-44.

In embodiments, the combination comprises tizanidine, a CB₁ agent, DOHx, and 5-CT. In embodiments, the combination comprises tizanidine, a CB₁ agent, DOHx, and 5-MeO-DALT. In embodiments, the combination comprises tizanidine, a CB₁ agent, DOHx, and 5-MeO-DMT In embodiments, the combination comprises tizanidine, a CB₁ agent, DOHx, and 5-MeO-MiPT. In embodiments, the combination comprises tizanidine, a CB₁ agent, DOHx, and AGH-192. In embodiments, the combination comprises tizanidine, a CB₁ agent, DOHx, and AGH-107. In embodiments, the combination comprises tizanidine, a CB₁ agent, DOHx, and AMT. In embodiments, the combination comprises tizanidine, a CB₁ agent, DOHx, and aripiprazole. In embodiments, the combination comprises tizanidine, a CB₁ agent, DOHx, and AS-19. In embodiments, the combination comprises tizanidine, a CB₁ agent, DOHx, and E-55888. In embodiments, the combination comprises tizanidine, a CB₁ agent, DOHx, and LSD. In embodiments, the combination comprises tizanidine, a CB₁ agent, DOHx, and LP-211. In embodiments, the combination comprises tizanidine, a CB₁ agent, DOHx, and LP-44.

In embodiments, the combination comprises tizanidine, a CB₁ agent, MEM, and 5-CT. In embodiments, the combination comprises tizanidine, a CB₁ agent, MEM, and 5-MeO-DALT. In embodiments, the combination comprises tizanidine, a CB₁ agent, MEM, and 5-MeO-DMT. In embodiments, the combination comprises tizanidine, a CB₁ agent, MEM, and 5-MeO-MiPT. In embodiments, the combination comprises tizanidine, a CB₁ agent, MEM, and AGH-192. In embodiments, the combination comprises tizanidine, a CB₁ agent, MEM, and AGH-107. In embodiments, the combination comprises tizanidine, a CB₁ agent, MEM, and AMT. In embodiments, the combination comprises tizanidine, a CB₁ agent, MEM, and aripiprazole. In embodiments, the combination comprises tizanidine, a CB₁ agent, MEM, and AS-19. In embodiments, the combination comprises tizanidine, a CB₁ agent, MEM, and E-55888. In embodiments, the combination comprises tizanidine, a CB₁ agent, MEM, and LSD. In embodiments, the combination comprises tizanidine, a CB₁ agent, MEM, and LP-211. In embodiments, the combination comprises tizanidine, a CB₁ agent, MEM, and LP-44.

In embodiments, the combination comprises tizanidine, a CB₁ agent, CPP, and 5-CT. In embodiments, the combination comprises tizanidine, a CB₁ agent, CPP, and 5-MeO-DALT. In embodiments, the combination comprises tizanidine, a CB₁ agent, CPP, and 5-MeO-DMT. In embodiments, the combination comprises tizanidine, a CB₁ agent, CPP, and 5-MeO-MiPT. In embodiments, the combination comprises tizanidine, a CB₁ agent, CPP, and AGH-192. In embodiments, the combination comprises tizanidine, a CB₁ agent, CPP, and AGH-107. In embodiments, the combination comprises tizanidine, a CB₁ agent, CPP, and AMT. In embodiments, the combination comprises tizanidine, a CB₁ agent, CPP, and aripiprazole. In embodiments, the combination comprises tizanidine, a CB₁ agent, CPP, and AS-19. In embodiments, the combination comprises tizanidine, a CB₁ agent, CPP, and E-55888. In embodiments, the combination comprises tizanidine, a CB₁ agent, CPP, and LSD. In embodiments, the combination comprises tizanidine, a CB₁ agent, CPP, and LP-211. In embodiments, the combination comprises tizanidine, a CB₁ agent, CPP, and LP-44.

In embodiments, the combination comprises tizanidine, a CB₁ agent, NBOH-2C-CN, and 5-CT. In embodiments, the combination comprises tizanidine, a CB₁ agent, NBOH-2C-CN, and 5-MeO-DALT. In embodiments, the combination comprises tizanidine, a CB₁ agent, NBOH-2C-CN, and 5-MeO-DMT. In embodiments, the combination comprises tizanidine, a CB₁ agent, NBOH-2C-CN, and 5-MeO-MiPT In embodiments, the combination comprises tizanidine, a CB₁ agent, NBOH-2C-CN, and AGH-192. In embodiments, the combination comprises tizanidine, a CB₁ agent, NBOH-2C-CN, and AGH-107. In embodiments, the combination comprises tizanidine, a CB₁ agent, NBOH-2C-CN, and AMT. In embodiments, the combination comprises tizanidine, a CB₁ agent, NBOH-2C-CN, and aripiprazole. In embodiments, the combination comprises tizanidine, a CB₁ agent, NBOH-2C-CN, and AS-19. In embodiments, the combination comprises tizanidine, a CB₁ agent, NBOH-2C-CN, and E-55888. In embodiments, the combination comprises tizanidine, a CB₁ agent, NBOH-2C-CN, and LSD. In embodiments, the combination comprises tizanidine, a CB₁ agent, NBOH-2C-CN, and LP-211. In embodiments, the combination comprises tizanidine, a CB₁ agent, NBOH-2C-CN, and LP-44.

In embodiments, the combination comprises tizanidine, a CB₁ agent, TCB-2, and 5-CT. In embodiments, the combination comprises tizanidine, a CB₁ agent, TCB-2, and 5-MeO-DALT. In embodiments, the combination comprises tizanidine, a CB₁ agent, TCB-2, and 5-MeO-DMT. In embodiments, the combination comprises tizanidine, a CB₁ agent, TCB-2, and 5-MeO-MiPT. In embodiments, the combination comprises tizanidine, a CB₁ agent, TCB-2, and AGH-192. In embodiments, the combination comprises tizanidine, a CB₁ agent, TCB-2, and AGH-107. In embodiments, the combination comprises tizanidine, a CB₁ agent, TCB-2, and AMT. In embodiments, the combination comprises tizanidine, a CB₁ agent, TCB-2, and aripiprazole. In embodiments, the combination comprises tizanidine, a CB₁ agent, TCB-2, and AS-19. In embodiments, the combination comprises tizanidine, a CB₁ agent, TCB-2, and E-55888. In embodiments, the combination comprises tizanidine, a CB₁ agent, TCB-2, and LSD. In embodiments, the combination comprises tizanidine, a CB₁ agent, TCB-2, and LP-211. In embodiments, the combination comprises tizanidine, a CB₁ agent, TCB-2, and LP-44.

In embodiments, the combination comprises tizanidine, a CB₁ agent, WAY 161503, and 5-CT. In embodiments, the combination comprises tizanidine, a CB₁ agent, WAY 161503, and 5-MeO-DALT. In embodiments, the combination comprises tizanidine, a CB₁ agent, WAY 161503, and 5-MeO-DMT In embodiments, the combination comprises tizanidine, a CB₁ agent, WAY 161503, and 5-MeO-MiPT. In embodiments, the combination comprises tizanidine, a CB₁ agent, WAY 161503, and AGH-192. In embodiments, the combination comprises tizanidine, a CB₁ agent, WAY 161503, and AGH-107. In embodiments, the combination comprises tizanidine, a CB₁ agent, WAY 161503, and AMT. In embodiments, the combination comprises tizanidine, a CB₁ agent, WAY 161503, and aripiprazole. In embodiments, the combination comprises tizanidine, a CB₁ agent, WAY 161503, and AS-19. In embodiments, the combination comprises tizanidine, a CB₁ agent, WAY 161503, and E-55888. In embodiments, the combination comprises tizanidine, a CB₁ agent, WAY 161503, and LSD. In embodiments, the combination comprises tizanidine, a CB₁ agent, WAY 161503, and LP-211. In embodiments, the combination comprises tizanidine, a CB₁ agent, WAY 161503, and LP-44.

In embodiments, wherein the combination comprises MDMA, a CB₁ agent, and optionally a 5-HT₂ and/or a 5-HT₇ agent, the CB₁ agent will be one or more of 2-AGE, A-834,735, ACEA, AZ-11713908, AZD-1940, CBN, CP-47497, CP55940, HU-210, JWH-007, JWH-051, JWH-146, JWH-176, JWH-200, JWH-398, Org 28611, rimonabant, THC, and XLR11.

In embodiments, wherein the combination comprises MDMA, a CB₁ agent, and optionally a 5-HT₂ and/or a 5-HT₇ agent, the CB₁ agent will not be WIN 55,212-2.

### J. Exemplary Combinations Comprising a Primer and Probe

In embodiments, the therapeutic combination or synergistic combination may be referred to as comprising a “primer” and a “probe.” In embodiments, when at least one primer and at least one probe of the invention are each administered in a therapeutically effective amount (e.g., in a therapeutic combination or in a pharmaceutical composition), they will elicit the subjective effects of an entactogenic mindstate.

In embodiments, primers are selected from the group consisting of CB₁ agents, 5-HT₂ agents, and 5-HT₇ agents. In embodiments, primers are selected from the group consisting of CB₁ agonists, 5-HT₂ agonists, and 5-HT₇ agonists. In embodiments, primers are selected from the group consisting of CB₁ antagonists, 5-HT₂ antagonists, and 5-HT₇ antagonists. In embodiments, primers include any of CB₁ agonists, CB₁ antagonists, 5-HT₂ agonists, 5-HT₂ antagonists, 5-HT₇ agonists, 5-HT₇ antagonists, or combinations thereof. Accordingly, in embodiments, any of CB₁ agents, 5-HT₂ agents, and 5-HT₇ agents may be used as and will be considered primers. In embodiments, any of CB₁ antagonists, 5-HT₂ antagonists, and 5-HT₇ antagonists may be used as and will be considered primers. In embodiments, any of CB₁ agonists, CB₁ antagonists, 5-HT₂ agonists, 5-HT₂ antagonists, 5-HT₇ agonists, 5-HT₇ antagonists, or combinations thereof may be used as and will be considered primers.

In embodiments, probes include I₁ agents. In embodiments, probes include I₁ agonists. In embodiments, probes include I₁ antagonists. In embodiments, probes include any of I₁ agonists, I₁ antagonists, or combinations thereof. Accordingly, in embodiments, I₁ agents may be used as and will be considered probes. In embodiments, I₁ agonists may be used as and will be considered probes. In embodiments, any of I₁ agonists, I₁ antagonists, or combinations thereof may be used as and will be considered probes.

In embodiments, a therapeutic combination of the invention comprises a probe that is an I₁ agent and a primer that is a CB₁ agent. In embodiments, a therapeutic combination of the invention comprises a probe that is an I₁ agonist and a primer that is a CB₁ agonist. In embodiments, a therapeutic combination of the invention comprises a probe that is an I₁ agonist and a primer that is a CB₁ antagonist. In embodiments, a therapeutic combination of the invention comprises a probe that is an I₁ antagonist and a primer that is a CB₁ agonist. In embodiments, a therapeutic combination of the invention comprises a probe that is an I₁ antagonist and a primer that is a CB₁ antagonist.

In embodiments, a therapeutic combination of the invention comprises a probe that is an I₁ agent and a primer that is a 5-HT₂ agent. In embodiments, a therapeutic combination of the invention comprises a probe that is an I₁ agonist and a primer that is a 5-HT₂ agonist. In embodiments, a therapeutic combination of the invention comprises a probe that is an I₁ agonist and a primer that is a 5-HT₂ antagonist. In embodiments, a therapeutic combination of the invention comprises a probe that is an I₁ antagonist and a primer that is a 5-HT₂ agonist. In embodiments, a therapeutic combination of the invention comprises a probe that is an I₁ antagonist and a primer that is a 5-HT₂ antagonist.

In embodiments, a therapeutic combination of the invention comprises a probe that is an I₁ agent and a primer that is a 5-HT₇ agent. In embodiments, a therapeutic combination of the invention comprises a probe that is an I₁ agonist and a primer that is a 5-HT₇ agonist. In embodiments, a therapeutic combination of the invention comprises a probe that is an I₁ agonist and a primer that is a 5-HT₇ antagonist. In embodiments, a therapeutic combination of the invention comprises a probe that is an I₁ antagonist and a primer that is a 5-HT₇ agonist. In embodiments, a therapeutic combination of the invention comprises a probe that is an I₁ antagonist and a primer that is a 5-HT₇ antagonist.

In embodiments, a therapeutic combination of the invention comprises a probe that is an I₁ agent, a primer that is a CB₁ agent, and a primer that is a 5-HT₂ agent. In embodiments, a therapeutic combination of the invention comprises a probe that is an I₁ agonist, a primer that is a CB₁ agonist, and a primer that is a 5-HT₂ agonist. In embodiments, a therapeutic combination of the invention comprises a probe that is an I₁ agonist, a primer that is a CB₁ agonist, and a primer that is a 5-HT₂ antagonist. In embodiments, a therapeutic combination of the invention comprises a probe that is an I₁ agonist, a primer that is a CB₁ antagonist, and a primer that is a 5-HT₂ agonist. In embodiments, a therapeutic combination of the invention comprises a probe that is an I₁ agonist, a primer that is a CB₁ antagonist, and a primer that is a 5-HT₂ antagonist. In embodiments, a therapeutic combination of the invention comprises a probe that is an I₁ antagonist, a primer that is a CB₁ agonist, and a primer that is a 5-HT₂ agonist. In embodiments, a therapeutic combination of the invention comprises a probe that is an I₁ antagonist, a primer that is a CB₁ agonist, and a primer that is a 5-HT₂ antagonist. In embodiments, a therapeutic combination of the invention comprises a probe that is an I₁ antagonist, a primer that is a CB₁ antagonist, and a primer that is a 5-HT₂ agonist. In embodiments, a therapeutic combination of the invention comprises a probe that is an I₁ antagonist, a primer that is a CB₁ antagonist, and a primer that is a 5-HT₂ antagonist.

In embodiments, a therapeutic combination of the invention comprises a probe that is an I₁ agent, a primer that is a CB₁ agent, and a primer that is a 5-HT₇ agent. In embodiments, a therapeutic combination of the invention comprises a probe that is an I₁ agonist, a primer that is a CB₁ agonist, and a primer that is a 5-HT₇ agonist. In embodiments, a therapeutic combination of the invention comprises a probe that is an I₁ agonist, a primer that is a CB₁ agonist, and a primer that is a 5-HT₇ antagonist. In embodiments, a therapeutic combination of the invention comprises a probe that is an I₁ agonist, a primer that is a CB₁ antagonist, and a primer that is a 5-HT₇ agonist. In embodiments, a therapeutic combination of the invention comprises a probe that is an I₁ agonist, a primer that is a CB₁ antagonist, and a primer that is a 5-HT₇ antagonist. In embodiments, a therapeutic combination of the invention comprises a probe that is an I₁ antagonist, a primer that is a CB₁ agonist, and a primer that is a 5-HT₇ agonist. In embodiments, a therapeutic combination of the invention comprises a probe that is an I₁ antagonist, a primer that is a CB₁ agonist, and a primer that is a 5-HT₇ antagonist. In embodiments, a therapeutic combination of the invention comprises a probe that is an I₁ antagonist, a primer that is a CB₁ antagonist, and a primer that is a 5-HT₇ agonist. In embodiments, a therapeutic combination of the invention comprises a probe that is an I₁ antagonist, a primer that is a CB₁ antagonist, and a primer that is a 5-HT₇ antagonist.

In embodiments, a therapeutic combination of the invention comprises a probe that is an I₁ agent, a primer that is a 5-HT₂ agent, and a primer that is a 5-HT₇ agent. In embodiments, a therapeutic combination of the invention comprises a probe that is an I₁ agonist, a primer that is a 5-HT₂ agonist, and a primer that is a 5-HT₇ agonist. In embodiments, a therapeutic combination of the invention comprises a probe that is an I₁ agonist, a primer that is a 5-HT₂ agonist, and a primer that is a 5-HT₇ antagonist. In embodiments, a therapeutic combination of the invention comprises a probe that is an I₁ agonist, a primer that is a 5-HT₂ antagonist, and a primer that is a 5-HT₇ agonist. In embodiments, a therapeutic combination of the invention comprises a probe that is an I₁ agonist, a primer that is a 5-HT₂ antagonist, and a primer that is a 5-HT₇ antagonist. In embodiments, a therapeutic combination of the invention comprises a probe that is an I₁ antagonist, a primer that is a 5-HT₂ agonist, and a primer that is a 5-HT₇ agonist. In embodiments, a therapeutic combination of the invention comprises a probe that is an I₁ antagonist, a primer that is a 5-HT₂ agonist, and a primer that is a 5-HT₇ antagonist. In embodiments, a therapeutic combination of the invention comprises a probe that is an I₁ antagonist, a primer that is a 5-HT₂ antagonist, and a primer that is a 5-HT₇ agonist. In embodiments, a therapeutic combination of the invention comprises a probe that is an I₁ antagonist, a primer that is a 5-HT₂ antagonist, and a primer that is a 5-HT₇ antagonist.

In embodiments, a therapeutic combination of the invention comprises a probe that is an I₁ agent, a primer that is a CB₁ agent, a primer that is a 5-HT₂ agent, and a primer that is a 5-HT₇ agent. In embodiments, a therapeutic combination of the invention comprises a probe that is an I₁ agonist, a primer that is a CB₁ agonist, a primer that is a 5-HT₂ agonist, and a primer that is a 5-HT₇ agonist. In embodiments, a therapeutic combination of the invention comprises a probe that is an I₁ agonist, a primer that is a CB₁ agonist, a primer that is a 5-HT₂ agonist, and a primer that is a 5-HT₇ antagonist. In embodiments, a therapeutic combination of the invention comprises a probe that is an I₁ agonist, a primer that is a CB₁ agonist, a primer that is a 5-HT₂ antagonist, and a primer that is a 5-HT₇ agonist. In embodiments, a therapeutic combination of the invention comprises a probe that is an I₁ agonist, a primer that is a CB₁ agonist, a primer that is a 5-HT₂ antagonist, and a primer that is a 5-HT₇ antagonist. In embodiments, a therapeutic combination of the invention comprises a probe that is an I₁ agonist, a primer that is a CB₁ antagonist, a primer that is a 5-HT₂ agonist, and a primer that is a 5-HT₇ agonist. In embodiments, a therapeutic combination of the invention comprises a probe that is an I₁ agonist, a primer that is a CB₁ antagonist, a primer that is a 5-HT₂ agonist, and a primer that is a 5-HT₇ antagonist. In embodiments, a therapeutic combination of the invention comprises a probe that is an I₁ agonist, a primer that is a CB₁ antagonist, a primer that is a 5-HT₂ antagonist, and a primer that is a 5-HT₇ agonist. In embodiments, a therapeutic combination of the invention comprises a probe that is an I₁ agonist, a primer that is a CB₁ antagonist, a primer that is a 5-HT₂ antagonist, and a primer that is a 5-HT₇ antagonist. In embodiments, a therapeutic combination of the invention comprises a probe that is an I₁ antagonist, a primer that is a CB₁ agonist, a primer that is a 5-HT₂ agonist, and a primer that is a 5-HT₇ agonist. In embodiments, a therapeutic combination of the invention comprises a probe that is an I₁ antagonist, a primer that is a CB₁ agonist, a primer that is a 5-HT₂ agonist, and a primer that is a 5-HT₇ antagonist. In embodiments, a therapeutic combination of the invention comprises a probe that is an I₁ antagonist, a primer that is a CB₁ agonist, a primer that is a 5-HT₂ antagonist, and a primer that is a 5-HT₇ agonist. In embodiments, a therapeutic combination of the invention comprises a probe that is an I₁ antagonist, a primer that is a CB₁ agonist, a primer that is a 5-HT₂ antagonist, and a primer that is a 5-HT₇ antagonist. In embodiments, a therapeutic combination of the invention comprises a probe that is an I₁ antagonist, a primer that is a CB₁ antagonist, a primer that is a 5-HT₂ agonist, and a primer that is a 5-HT₇ agonist. In embodiments, a therapeutic combination of the invention comprises a probe that is an I₁ antagonist, a primer that is a CB₁ antagonist, a primer that is a 5-HT₂ agonist, and a primer that is a 5-HT₇ antagonist. In embodiments, a therapeutic combination of the invention comprises a probe that is an I₁ antagonist, a primer that is a CB₁ antagonist, a primer that is a 5-HT₂ antagonist, and a primer that is a 5-HT₇ agonist. In embodiments, a therapeutic combination of the invention comprises a probe that is an I₁ antagonist, a primer that is a CB₁ antagonist, a primer that is a 5-HT₂ antagonist, and a primer that is a 5-HT₇ antagonist.

In embodiments, the I₁ agent is selected from the group consisting of agmatine, BDBM50091347, clonidine, guanfacine, mCPP, MDMA, memantine, moxonidine, naphazoline, oxymetazoline, rilmenidine, tetryzoline, tolonidine, and tizanidine.

In embodiments, the CB₁ agent is any of 2-AGE, A-834,735, ACEA, AZ-11713908, AZD-1940, CBN, CP-47497, CP55940, HU-210, JWH-007, JWH-051, JWH-146, JWH-176, JWH-200, JWH-398, Org 28611, rimonabant, THC, WIN 55,212-2, and XLR11.

In embodiments, the 5-HT₂ agent is any of 1-methylpsilocin, 2C-B-FLY, 5-MeO-AMT, AL-37350A, CP 809101, DALT, DOB, DOET, DOHx, MEM, CPP, NBOH-2C-CN, TCB-2, and WAY 161503.

In embodiments, the 5-HT₇ agent is any of 5-CT, 5-MeO-DALT, 5-MeO-DMT, 5-MeO-MiPT, AGH-192, AGH-107, AMT, aripiprazole, AS-19, E-55888, LSD, LP-211, and LP-44.

In some embodiments, certain compounds may be excluded from a disclosed entactogenic combination. For example, in some embodiments, where the I₁ agent is rilmenidine or moxonidine, the 5-HT₂ agent is not 2C-B-FLY, MEM, DOB, or DOET. In some embodiments, where the I₁ agent is rilmenidine or moxonidine, the 5-HT₂ agent is not 2C-B-FLY, MEM, DOB, or DOET, unless the I₁ agent and 5-HT₂ agent are formulated together with a pharmaceutically acceptable carrier, diluent, or excipient. In some embodiments, where the I₁ agent is rilmenidine or moxonidine, the 5-HT₂ agent is not 2C-B-FLY, MEM, DOB, or DOET, unless the I₁ agent and 5-HT₂ agent are formulated together with a pharmaceutically acceptable carrier, diluent, or excipient, and prepared as a unit dosage form for immediate release, controlled release, sustained release, extended release, or modified release. In some embodiments, where the I₁ agent is rilmenidine or moxonidine, the 5-HT₂ agent is not 2C-B-FLY, MEM, DOB, or DOET, unless the I₁ agent and 5-HT₂ agent are used together in a method to treat a CNS disorder or condition. In some embodiments, where the I₁ agent is rilmenidine or moxonidine, the 5-HT₂ agent is not 2C-B-FLY, MEM, DOB, or DOET, unless the I₁ agent and 5-HT₂ agent are used together in a method to improve mental health or functioning. In some embodiments, where the I₁ agent is rilmenidine or moxonidine, the 5-HT₂ agent is not 2C-B-FLY, MEM, DOB, or DOET, unless the I₁ agent and 5-HT₂ agent are used together, and administered together with one or more sessions of psychotherapy.

In embodiments, the amount of primer and the probe are equivalent in a disclosed entactogenic combination. In embodiments, the amount of the primer is greater than the amount of the probe in a disclosed entactogenic combination. In embodiments, the amount of the primer is less than the amount of the probe in a disclosed entactogenic combination. In embodiments, the amount of primer relative to the amount of probe is expressed in a weight by weight ratio (w/w). In embodiments, the primer comprises one or more compounds as described herein, and the weight ratio is determined by combining the weights of such compounds. In embodiments, the probe comprises one or more compounds as described herein, and the weight ratio is determined by combining the weights of such compounds. In embodiments, the primer to probe ratio (w/w) is from 250:1 to 1:50, inclusive, in a disclosed entactogenic combination. In embodiments, the combinations of primer and probe provided herein effectively elicit an entactogenic mindstate.

In embodiments, the amount (w/w) of primer is greater than the amount (w/w) of probe. In embodiments, the primer to probe ratio (w/w) is from 35,000:1 to 1.5:1, inclusive. In embodiments, the primer to probe ratio (w/w) is 30,000:1, 25,000:1, 20,000:1, 15,000:1, 10,000:1, 5,000:1, 1,000:1, 900:1, 800:1, 700:1, 600:1, 500:1, 400:1, 300:1, 200:1, 190:1, 180:1, 170:1, 160:1, 150:1, 145:1, 140:1, 135:1, 130:1, 125:1, 120:1, 115:1, 110:1, 105:1, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, or 1.5:1, including values in between.

In embodiments, the primer to probe ratio (w/w) is from 135:1 to 85:1, inclusive. In embodiments, the primer to probe ratio (w/w) is 135:1, 134:1, 133:1, 132:1, 131:1, 130:1, 129:1, 128:1, 127:1, 126:1, 125:1, 124:1, 123:1, 122:1, 121:1, 120:1, 119:1, 118:1, 117:1, 116:1, 115:1, 114:1, 113:1, 112:1, 111:1, 110:1, 109:1, 108:1, 107:1, 106:1, 105:1, 104:1, 103:1, 102:1, 101:1, 100:1, 99:1, 98:1, 97:1, 96:1, 95:1, 94:1, 93:1, 92:1, 91:1, 90:1, 89:1, 88:1, 87:1, 86:1, or 85:1.

In embodiments, the primer to probe ratio (w/w) is from 50:1 to 1.1:1, inclusive. In embodiments, the primer to probe ratio (w/w) is 50:1, 49:1, 48:1, 47:1, 46:1, 45:1, 44:1, 43:1, 42:1, 41:1, 40:1, 39:1, 38:1, 37:1, 36:1, 35:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1:13:1, 12:1, 11:1, 10:1, 9:1, 8:1, 7:1, 6:1, 5:1, 4:1, 3:1, 2:1, 1.5:1, 1.4:1, 1.3:1, 1.2:1, or 1.1:1, including values in between.

In embodiments, the amount (w/w) of primer is equivalent to or less than the amount (w/w) of probe. In embodiments, the primer to probe ratio (w/w) is from 1:1 to 1:35,000, inclusive. In embodiments, the primer to probe ratio (w/w) is 1:1, 1:1.5, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, 1:105, 1:110, 1:120, 1:130, 1:135, 1:140, 1:145, 1:150, 1:160, 1:170, 1:180, 1:190, 1:200, 1:300, 1:400, 1:500, 1:600, 1:700, 1:800, 1:900, 1:1000, 1:5,000, 1:10,000, 1:15,000, 1:20,000, 1:25,000, 1:30,000, or 1:35,000, including values in between.

In embodiments, the primer to probe ratio (w/w) is from 1:135 to 1:85, inclusive. In embodiments, the primer to probe ratio (w/w) is 1:135, 1:134, 1:133, 1:132, 1:131, 1:130, 1:129, 1:128, 1:127, 1:126, 1:125, 1:124, 1:123, 1:122, 1:121, 1:120, 1:119, 1:118, 1:117, 1:116, 1:115, 1:114, 1:113, 1:112, 1:111, 1:110, 1:109, 1:108, 1:107, 1:106, 1:105, 1:104, 1:103, 1:102, 1:101, 1:100, 1:99, 1:98, 1:97, 1:96, 1:95, 1:94, 1:93, 1:92, 1:91, 1:90, 1:89, 1:88, 1:87, 1:86, or 1:85.

In embodiments, the primer to probe ratio (w/w) is from 1:50 to 1.1:1, inclusive. In embodiments, the primer to probe ratio (w/w) is 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, or 1:1.1.

### K. Compounds Used in the Therapeutic Combinations

Certain compounds described herein may contain one or more asymmetric centers and give rise to enantiomers, diastereomers, and other stereoisomeric forms. Each chiral center may be defined, in terms of absolute stereochemistry, as (R)- or (S)-. The invention is meant to include all such possible isomers, as well as mixtures thereof, including racemic and optically pure forms. Thus, for example, chiral compounds will be understood to refer to racemic mixtures, as well as any of the individual enantiomers, and all enantiomerically enriched mixtures thereof, in any proportions.

Optically active (R)- and (S)-, (−)- and (+)-, or (D)- and (L)-isomers may be prepared using chiral synthons or chiral reagents, or resolved using conventional techniques. Various methods are known in the art for preparing optically active forms and determining activity. Such methods include standard tests described herein and other similar tests which are well known in the art. Examples of methods that can be used to obtain optical isomers of the compounds according to the present disclosure include the following: i) physical separation of crystals whereby macroscopic crystals of the individual enantiomers are manually separated. This technique may be particularly useful if crystals of the separate enantiomers exist (i.e., the material is a conglomerate), and the crystals are visually distinct; ii) simultaneous crystallization whereby the individual enantiomers are separately crystallized from a solution of the racemate, possible only if the latter is a conglomerate in the solid state; iii) enzymatic resolutions whereby partial or complete separation of a racemate by virtue of differing rates of reaction for the enantiomers with an enzyme; iv) enzymatic asymmetric synthesis, a synthetic technique whereby at least one step of the synthesis uses an enzymatic reaction to obtain an enantiomerically pure or enriched synthetic precursor of the desired enantiomer; v) chemical asymmetric synthesis whereby the desired enantiomer is synthesized from an achiral precursor under conditions that produce asymmetry (i.e., chirality) in the product, which may be achieved using chiral catalysts or chiral auxiliaries; vi) diastereomer separations whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer; vii) first- and second-order asymmetric transformations whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomers; viii) kinetic resolutions comprising partial or complete resolution of a racemate (or of a further resolution of a partially resolved compound) by virtue of unequal reaction rates of the enantiomers with a chiral, non-racemic reagent or catalyst under kinetic conditions; ix) enantiospecific synthesis from non-racemic precursors whereby the desired enantiomer is obtained from non-chiral starting materials and where the stereochemical integrity is not or is only minimally compromised over the course of the synthesis; x) chiral liquid chromatography whereby the enantiomers of a racemate are separated in a liquid mobile phase by virtue of their differing interactions with a stationary phase. The stationary phase can be made of chiral material or the mobile phase can contain an additional chiral material to provoke the differing interactions; xi) chiral gas chromatography whereby the racemate is volatilized and enantiomers are separated by virtue of their differing interactions in the gaseous mobile phase with a column containing a fixed non-racemic chiral adsorbent phase; xii) extraction with chiral solvents whereby the enantiomers are separated by virtue of preferential dissolution of one enantiomer into a particular chiral solvent; and xiii) transport across chiral membranes whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane, which allows only one enantiomer of the racemate to pass through.

The disclosed compounds may be provided in a composition that is enantiomerically enriched, such as a mixture of enantiomers in which one enantiomer is present in excess, in particular to the extent of at least 50%, at least 55%, at least 60%, at least 65%, at least 70%, at least 75%, at least 80%, at least 85%, at least 90%, at least 95%, at least 98%, at least 99%, at least 99.5%, at least 99.9%, up to (and including) 100%, and values in between.

When the compounds described herein contain olefinic double bonds or other centers of geometric asymmetry, and unless specified otherwise, it is intended that the compounds include both E and Z geometric isomers. Likewise, tautomeric forms are included.

The individual compounds of the compositions of the invention will be understood to also encompass pharmaceutically acceptable salts of such compounds. The term “pharmaceutically acceptable salt” may refer to salts prepared from pharmaceutically acceptable non-toxic acids or bases, and which may be synthesized by conventional chemical methods. Generally, such salts are prepared by reacting the free acid or base forms of these agents with a stoichiometric amount of the appropriate base or acid in water or in an organic solvent, or in a mixture of the two; generally, nonaqueous media (e.g., ether, ethyl acetate, ethanol, isopropanol, or acetonitrile) are preferred. For therapeutic use, salts of the compounds are those wherein the counter-ion is pharmaceutically acceptable.

Exemplary salts include, but are not limited to, 2-hydroxyethanesulfonate, 2-naphthalenesulfonate, 2-napsylate, 3-hydroxy-2-naphthoate, 3-phenylpropionate, 4-acetamidobenzoate, acefyllinate, acetate, aceturate, adipate, alginate, aminosalicylate, ammonium, amsonate, ascorbate, aspartate, benzenesulfonate, benzoate, besylate, bicarbonate, bisulfate, bitartrate, borate, butyrate, calcium edetate, calcium, camphocarbonate, camphorate, camphorsulfonate, camsylate, carbonate, cholate, citrate, clavulariate, cyclopentanepropionate, cypionate, d-aspartate, d-camsylate, d-lactate, decanoate, dichloroacetate, digluconate, dodecylsulfate, edentate, edetate, edisylate, estolate, esylate, ethanesulfonate, ethyl sulfate, fumarate, furate, fusidate, galactarate (mucate), galacturonate, gallate, gentisate, gluceptate, glucoheptanoate, gluconate, glucuronate, glutamate, glutarate, glycerophosphate, glycolate, glycollylarsanilate, hemisulfate, heptanoate (enanthate), heptanoate, hexafluorophosphate, hexanoate, hexylresorcinate, hippurate, hybenzate, hydrabamine, hydrobromide, hydrobromide/bromide, hydrochloride/chloride, hydrofluoride/fluoride, hydroiodide, hydroxide, hydroxybenzoate, hydroxynaphthoate, iodide, isethionate, isothionate, I-aspartate, I-camsylate, I-lactate, lactate, lactobionate, laurate, laurylsulphonate, lithium, magnesium, malate, maleate, malonate, mandelate, meso-tartrate, mesylate, methanesulfonate, methyl bromide, methyl nitrate, methylsulfate, mucate, myristate, N-methylglucamine ammonium salt, napadisilate, naphthylate, napsylate, nicotinate, nitrate, octanoate, oleate, orotate, oxalate, p-toluenesulfonate, palmitate, pamoate, pantothenate, pectinate, persulfate, phenylpropionate, phosphate, phosphateldiphosphate, picrate, pivalate, polygalacturonate, potassium, propionate, pyrophosphate, saccharate, salicylate, salicylsulfate, sodium, stearate, subacetate, succinate, sulfate, sulfosaliculate, sulfosalicylate, suramate, tannate, tartrate, teoclate, terephthalate, thiocyanate, thiosalicylate, tosylate, tribrophenate, triethiodide, undecanoate, undecylenate, valerate, valproate, xinafoate, zinc and the like, as would be immediately evident to one of skill. (See Berge et al. (1977) “Pharmaceutical Salts,” J. Pharm. Sci. 66:1-19.) Preferred pharmaceutically acceptable salts in some embodiments are those employing a hydrochloride anion. In other embodiments, a preferred salt may be a fumarate salt, or a succinate salt.

Different embodiments include the following examples: Pharmaceutically acceptable complex derivatives of each drug in each group, including solvates, salts, esters, enantiomers, isomers (stereoisomers and/or constitutional, including ones based on substituting deuterium or a halogen, such as bromine, chlorine, fluorine, and iodine; for any hydrogen), derivatives or prodrugs of the disclosed compounds.

Prodrugs of the active agents also will be appreciated to be within the scope of the disclosure. The term “prodrug” may refer to a precursor of a biologically active pharmaceutical agent. Prodrugs undergo a chemical or a metabolic conversion to become a biologically active pharmaceutical agent. A prodrug can be converted ex vivo to the biologically active pharmaceutical agent by chemical transformative processes. In vivo, a prodrug is converted to the biologically active pharmaceutical agent by the action of a metabolic process, an enzymatic process or a degradative process that removes the prodrug moiety, such as a glycoside or acetyl group, to form the biologically active pharmaceutical agent. Other examples include addition of hydroxy groups (Tsujikawa et al. 2011. Xenobiotica, 41(7), 578-584; Yamamoto et al. 1984. Xenobiotica, 14(11), 867-875), acyloxyalkoxycarbonyl derivatives, amino acids, or peptides (Vig et al. 2013. Advanced Drug Delivery Reviews, 65(10), 1370-1385), which are generally added to the amine, and can be removed within the body by chemical reactions or enzymes, but other prodrugs and precursors, at the amine and other sites, should be understood to be within the scope of the disclosure (Simplicio, Clancy, & Gilmer. 2008. Molecules, 13(3), 519-547; Shah, Chauhan, Chauhan, & Mishra (Eds.). 2020. Recent Advancement in Prodrugs. CRC Press).

Types of prodrugs contemplated to be within the scope and spirit of the invention therefore include compounds that are transformed in various organs or locations in the body (e.g., liver, kidney, G.I., lung, tissue) to release the active agent. For example, liver prodrugs will include active agents conjugated with a polymer or chemical moiety that is not released until acted upon by liver cytochrome enzymes; CYP metabolism includes dealkylation, dehydrogenation, reduction, hydrolysis, oxidation, and the breakdown of aromatic rings. Kidney prodrugs will include active agents conjugated to L-gamma-glutamyl or N-acetyl-L-gamma glutamic moieties so that they are metabolized by gamma-glutamyl transpeptidase before they are bioactive; alternatively, they may be conjugated to alkylglucoside moieties to create glycosylation-based prodrugs. Digestive or G.I. prodrugs will include those where an active agent is, e.g., formulated into microspheres or nanospheres that do not degrade until the spheres are subjected to an acidic pH; formulated with an amide that will resist biochemical degradation until colonic pH is achieved; or conjugated with a linear polysaccharide such as pectin that will delay activation until the pharmaceutical composition reaches the bacteria in the colon. Besides these exemplary prodrug forms, many others will be known to those of skill.

In the case of solid compositions, it is understood that the compounds used in the disclosed methods may exist in different forms. For example, the compounds may exist in stable and metastable crystalline forms, isotropic and amorphous forms, milled forms and nano-particulate forms, all of which are intended to be within the scope of the present invention. In addition, compounds described herein include crystalline forms, also known as polymorphs. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility.

Among derivatives of a compound are included its “physiologically functional derivatives,” which may refer to physiologically tolerated chemical derivatives of the compound having the same physiological function thereof, for example, by being convertible in the body thereto, and which on administration to a mammal such as a human is able to form (directly or indirectly) the compound or an active metabolite thereof (acting therefore, like a prodrug), or by otherwise having the same physiological function, despite one or more structural differences. According to the invention, examples of physiologically functional derivatives include esters, amides, carbamates, ureas, and heterocycles.

In embodiments, the disclosed compounds are synthetic. “Synthetic” may refer to a compound which is manufactured artificially in a laboratory, by means of chemical synthesis or biosynthesis.

In other embodiments, the disclosed compounds are extracted from natural sources, and purified or isolated therefrom. For example, DMT may be extracted from, among other sources, including but not limited to Phalaris, Delosperma, Acacia, Desmodium, Mimosa, Virola, and Psychotria spp. In general, “natural” means a compound that was isolated, extracted, or otherwise obtained from a natural source, such as a plant, animal, or mineral. Methods of extracting compounds from plants, and creating plant extracts, and methods of obtaining purified products containing the desired compounds free from undesired plant matter, chemicals, and other impurities, are known in the art.

Generally, the individual disclosed compounds shall be administered as part of a pharmaceutical composition or formulation, but will be prepared for inclusion in such composition or formulations as isolated or purified compounds. In certain preferred embodiments, a disclosed compound is in a substantially pure or isolated preparation. The terms “isolated,” “purified,” or “substantially pure,” as used herein, refer to material that is substantially or essentially free from components that normally accompany the material when the material is synthesized, manufactured, or otherwise produced, according to a particular degree of purity, and when measured by a method described herein or of general knowledge in the art.

A compound of the disclosure can be purified. A compound (or preparation of a compound) may be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, at least 99.9% pure, or up to 100% pure.

A “substantially pure” compound (or a preparation of a compound) may be defined as a compound or preparation having a chromatographic purity (of the desired compound) of greater than 90%, more preferably greater than 95%, more preferably greater than 96%, more preferably greater than 97%, more preferably greater than 98%, more preferably greater than 99%, more preferably greater than 99.25%, more preferably greater than 99.50%, more preferably greater than 99.75%, and most preferably greater than 99.90%, greater than 99.95%, or greater than 99.99%, as determined by area normalization of HPLC profile or similar detection method.

A substantially pure compound used in the invention is, preferably, substantially free of any other active agents which are not intended to be administered to a subject. In this context “substantially free” can be taken to mean that no active agent(s) other than the active agent intended to be administered to a subject are detectable by HPLC or other similar detection method, or are below a desired threshold of detection such as defined above.

The disclosed compounds now generally described will be more readily understood by reference to the following description and examples, which are included for the purposes of illustration of certain aspects of the embodiments. The following is not intended to limit the invention, as one of skill in the art would recognize from the teachings and examples herein that other techniques and methods can satisfy the claims and be employed without departing from the scope of the disclosure. Indeed, while this invention has been particularly shown and described with reference to certain exemplary embodiments, it will be understood by those skilled in the art that various changes in form and details may be made without departing from the scope or spirit of the invention encompassed by the appended claims.

### L. Additional Active Agents

The therapeutic combinations of the invention can also be used together with one or more additional active agents, which may, for example, contribute to or provide an additional therapeutic effect, or contribute to or provide a synergistic effect.

For example, in embodiments the therapeutic combinations are used in further combination with one or more additional therapeutically active ingredients (an “additional active agent”) such as SSRIs, SNRIs, NDRIs, TCAs, benzodiazepines, atypical antipsychotics, stimulants such as amphetamines and methylphenidate, ketamine, classical psychedelics such as mescaline, lysergic acid diethylamide (LSD), psilocybin and N,N-dimethyltryptamine (DMT) (i.e., where such additional active agent(s) are not already part of a disclosed entactogenic combination). In embodiments, the one or more additional active agents may be administered in separate dosage forms or as a single dosage form comprising one or more compounds according to the invention in combination with one or more additional active agents. For instance, due to the slow onset of an SSRI as described above, it might be beneficial in some instances to initiate the treatment with one or more compounds according to the invention together with, or followed by, a SSRI to avoid any delay in antidepressant effect.

In embodiments, an additional active agent is any one or more of amino acids, antioxidants, anti-inflammatory agents, analgesics, antineuropathic and antinociceptive agents, antimigraine agents, anxiolytics, antidepressants, antipsychotics, anti-PTSD agents, cannabinoids, dissociatives, immunostimulants, anti-cancer agents, antiemetics, orexigenics, antiulcer agents, antihistamines, anti hypertensives, anticonvulsants, antiepileptics, bronchodilators, neuroprotectants, entactogens and empathogens, entheogens, psychedelics, monoamine oxidase inhibitors, tryptamines, terpenes, phenethylamines, sedatives, serotonergic agents, stimulants, and vitamins. Such agents may be in ion, freebase, or salt form, and may be isomers, prodrugs, derivatives (preferably physiologically functional derivatives), or analogs.

In embodiments, the additional active agent is a serotonergic agent. A “serotonergic agent” may refer to a compound that binds to, blocks, activates, inhibits, or otherwise influences (e.g., via an allosteric reaction) activity at one or more serotonin receptors, including any one or more serotonin receptor subtypes. In embodiments, a serotonergic agent binds to a serotonin receptor. In embodiments, a serotonergic agent indirectly affects a serotonin receptor, e.g., via interactions affecting the reactivity of other molecules at the serotonin receptor. In embodiments, a serotonergic agent is an agonist, e.g., a compound activating a serotonin receptor. In embodiments, a serotonergic agent is an antagonist, e.g., a compound binding to but not activating a serotonin receptor, e.g., blocking a receptor. In embodiments, a serotonergic agent is an effector molecule, e.g., a compound binding to an enzyme for allosteric regulation. In embodiments, a serotonergic agent acts (either directly or indirectly) at more than one type of receptor, including receptors other than serotonergic or other monoaminergic receptors. In embodiments, a serotonergic agent blocks the serotonin transporter (SERT) and results in an elevation of the synaptic concentration of serotonin, and an increase of neurotransmission. In embodiments, a serotonergic agent acts as a reuptake modulator and inhibits the plasmalemmal transporter-mediated reuptake of serotonin from the synapse into the presynaptic neuron, leading to an increase in extracellular concentrations of serotonin and an increase in neurotransmission. In embodiments, a serotonergic agent inhibits the activity of one or both monoamine oxidase enzymes, resulting in an increase in concentrations of serotonin and an increase in neurotransmission. In embodiments, a serotonergic agent is an antidepressant or anxiolytic, such as an SSRI, serotonin-norepinephrine reuptake inhibitor (SNRI), tricyclic antidepressant (TCA), monoamine oxidase inhibitor (MAOI), or atypical antidepressant. In other embodiments, a serotonergic agent is selected from the group consisting of: (1) serotonin transport inhibitors; (2) serotonin receptor modulators; (3) serotonin reuptake inhibitors; (4) serotonin and norepinephrine reuptake inhibitors; (5) serotonin dopamine antagonists; (6) monoamine reuptake inhibitors; (7) pyridazinone aldose reductase inhibitors; (8) stimulants of serotonin receptors; (9) stimulants of serotonin synthesis; (10) serotonin receptor agonists; (11) serotonin receptor antagonists; and (12) serotonin metabolites.

### M. Pharmaceutical Compositions

While it is possible to administer a compound employed in the disclosed methods directly without any formulation, the compounds are usually administered in the form of a pharmaceutical composition.

A therapeutic combination may comprise multiple such compositions, e.g., wherein two disclosed compounds are administered separately, such as simultaneously or sequentially, including at some distance from each other in time, such as within 1 minute, within 5 minutes, within 10 minutes, within 15 minutes, within 30 minutes, within 1 hour, within 2 hours, within 3 hours, within 6 hours, within 12 hours, within 24 hours, within 36 hours, within 48 hours, within 72 hours, or within 1 week, including times in between.

In embodiments, two or more disclosed compounds are formulated as a single composition, e.g., as a fixed-dose combination. A “fixed-dose combination” (“FDC”) may refer to a combination drug product comprising two or more active drugs in a single dosage form (e.g., a “single pill combination” or “SPC”).

In embodiments herein, two or more compounds or compositions thus may be administered simultaneously, concurrently, or sequentially. Simultaneous administration, in embodiments, may be carried out by mixing the compounds prior to administration or by administering the compounds at the same point in time but at different anatomic sites or using different routes of administration. Terms such as “concurrent administration,” “co-administration,” “simultaneous administration,” and “administered simultaneously” mean that the compounds are administered in combination, and comprise a “therapeutic combination.”

“Pharmaceutical compositions” are compositions that include the disclosed compound(s) together in an amount (for example, in a unit dosage form) with a pharmaceutically acceptable carrier, diluent, or excipient. It should be understood that embodiments do not have a single carrier, diluent, or excipient alone, but include multiple carriers, diluents, and/or excipients. Compositions can be prepared by standard pharmaceutical formulation techniques such as disclosed in, e.g., Remington: The Science and Practice of Pharmacy (2020) 23th ed., Academic Press., Cambridge, Mass.; The Merck Index (1996) 12th ed., Merck Publishing Group, Whitehouse, N.J.; Pharm. Principles of Solid Dosage Forms (1993), Technomic Publishing Co., Inc., Lancaster, Pa.; and Ansel and Stoklosa, Pharm. Calculations (2001) 11th ed., Lippincott Williams & Wilkins, Baltimore, Md.; and Poznansky et al. Drug Delivery Systems (1980), R. L. Juliano, ed., Oxford, N.Y., pp. 253-315).

“Pharmaceutically acceptable” as used in connection with a carrier, diluent, excipient, or other ingredient means that the ingredient is generally safe and, within the scope of sound medical judgment, suitable for use in contact with the cells of humans and other animals without undue toxicity, irritation, allergic response, or complication, and commensurate with a reasonable risk/benefit ratio.

In embodiments, a therapeutically effective amount of a disclosed entactogenic combination is administered to a patient in need thereof. A “therapeutically effective amount” or an “effective amount” refers to an amount of an active agent that is sufficient to provide a desired therapeutic effect. An effective amount will vary depending upon the subject and the disease condition being treated or the health benefit sought, the weight and age of the subject, the severity of the disease condition or degree of health benefit sought, the manner of administration, and the like.

Herein, “therapeutic effect” or “therapeutic efficacy” may refer to the responses(s) in a mammal, and preferably a human, after treatment that is judged to be desirable and/or beneficial. Hence, depending on the disorder to be treated, or improvement in physiological or psychological functioning sought, and depending on the particular constituent(s) in the compositions of the invention under consideration, those responses shall differ, but would be readily understood by those of skill. In preferred embodiments, and where the therapeutic effect is specifically an entactogenic “mindstate” or other mental state, the mammal generally will be understood to be a human.

Measures of therapeutic effect include outcome measures (primary or secondary), endpoints, effect measures, and measures of effect within clinical or medical practice or research which can be used to assess an effect (positive and/or negative) of an intervention or treatment, whether patient-reported (e.g., questionnaires); based on other patient data (e.g., patient monitoring); gathered through laboratory tests such as from blood or urine; through medical examination by a doctor or other medical professional, or by digital means, such as by using electronic tools such as online tools, smartphones, wireless devices, biosensors, or health apps.

“Therapeutically effective dose” may refer to the dose necessary to elicit a desired result within a patient undergoing treatment. A therapeutically effective dose therefore, in embodiments, may refer to a dose of any of a CB₁ agent, a 5-HT₂ agent, a 5-HT₇ agent, and/or an I₁ agent necessary to deliver measurable patient-specific biologic effects in the treatment or prevention of a disease/condition or creation of an “entactogenic mindstate” useful in doing the same. A therapeutically effective dose may also be referred to as a “therapeutically effective amount” and an “effective amount.”

Regarding administration, pharmaceutical compositions can be administered by a variety of routes, non-limiting examples of which include enteral means, wherein “enteral means” or “enteral administration means” includes, but is not limited to oral solid and oral liquid dosage forms, sublingual, rectal, and buccal administration means; as well as parenteral means, wherein “parenteral means” or “parenteral administration means” includes but is not limited to, intravenous, intra-arterial, intraperitoneal, intraosseous, intramuscular, intrathecal, intracerebroventricular, vaginal, ocular, nasal, cutaneous, topical, otic, ocular, transdermal, and subcutaneous administration means. As would be immediately appreciated by one of skill, the compounds employed in the disclosed methods are effectively administered as oral solid and oral liquid dosage forms, sublingually, buccally, as injections, including but not limited to, intravenous, intra-arterial, intraperitoneal, intraosseous, intramuscular, intrathecal, and intracerebroventricular; rectally, vaginally, ocularly, nasally, cutaneously, topically, optically, transdermally, and subcutaneously. Additionally, the compounds employed in the disclosed methods are effectively administered via any other means, and prepared as any acceptable compositions known to those of skill. Such compositions are prepared in a manner known in the pharmaceutical art and comprise at least one active agent (e.g., Remington, 2020.)

In making the compositions employed in the invention, the active ingredient is, in embodiments, mixed with an excipient, diluted by an excipient, or enclosed within such a carrier which can be in the form of a capsule, sachet, paper or other container. When the excipient serves as a diluent, it can be a solid, semi-solid, or liquid material, which acts as a vehicle, carrier, or medium for the active ingredient. Thus, the compositions can be in the form of tablets (including orally disintegrating, swallowable, sublingual, buccal, and chewable tablets), pills, powders, lozenges, troches, oral films, thin strips, sachets, cachets, elixirs, suspensions, emulsions, microemulsions, liposomal dispersions, aqueous and non-aqueous solutions, slurries, syrups, aerosols (as a solid or in a liquid medium), ointments containing, for example, up to 10% by weight of the active agent; soft and hard gelatin capsules, suppositories, dry powders for inhalation, liquid preparations for vaporization and inhalation, topical preparations, transdermal patches, transdermal gels, sterile injectable solutions, and sterile packaged powders. Compositions may be formulated as immediate release, timed-release, controlled release, sustained (extended) release or modified release formulations.

Some non-limiting examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations may additionally include at least one of: lubricating agents such as talc, magnesium stearate, and mineral oil; wetting agents; emulsifying and suspending agents; preserving agents such as methyl- and propylhydroxybenzoates. The compositions of the invention can be formulated so as to provide quick, sustained or delayed release of the active ingredient after administration to the patient by employing procedures known in the art.

In embodiments are multiple variations in the dosages of each drug in a disclosed entactogenic combination as further outlined below. In embodiments are various forms of preparations including solids, liquids, and immediate, delayed, or extended-release forms. Many types of variations are possible as known to those in the art.

In some embodiments, multiple routes of administration are included, which are patient-dependent and may differ according to patient preference, comorbidities, side effect profile, pharmacokinetic and pharmacodynamic considerations, and other factors (IV, PO, transdermal, etc.). Some embodiments include the presence of other substances with the active drugs, known to those skilled in the art, such as fillers, carriers, gels, skin patches, lozenges, or other modifications in the preparation to facilitate absorption through various routes (such as gastrointestinal, transdermal, etc.) and/or to extend the effect of the drugs, and/or to attain higher or more stable serum levels or to enhance the therapeutic effect of the active drugs in the pharmaceutical composition.

In preparing a formulation, it may be necessary to mill the active agent to provide the appropriate particle size prior to combining with the other ingredients. If the active agent is substantially insoluble, it ordinarily is milled to a particle size of less than 200 mesh. If the active agent is substantially water soluble, the particle size is normally adjusted by milling to provide a substantially uniform distribution in the formulation, e.g., 40 mesh.

In some embodiments, a specific size of the active agent may be desired. In embodiments, the active agent is less than 3 μm in diameter, less than 2 μm in diameter, less than 1 μm in diameter. In embodiments, the active agent is between 0.1 μm in diameter to 3 μm in diameter. In embodiments, the active agent is between 0.5 μm in diameter to 1.5 μm in diameter. In embodiments, the active agent is 0.1 μm in diameter, 0.2 μm in diameter, 0.25 μm in diameter, 0.3 μm in diameter, 0.35 μm in diameter, 0.4 μm in diameter, 0.45 μm in diameter, 0.5 μm in diameter, 0.55 μm in diameter, 0.6 μm in diameter, 0.65 μm in diameter, 0.7 μm in diameter, 0.75 μm in diameter, 0.8 μm in diameter, 0.85 μm in diameter, 0.9 μm in diameter, 0.95 μm in diameter, 1 μm in diameter, 1.1 μm in diameter, 1.2 μm in diameter, 1.3 μm in diameter, 1.4 μm in diameter, 1.5 μm in diameter, 1.6 μm in diameter, 1.7 μm in diameter, 1.8 μm in diameter, 1.9 μm in diameter, 2 μm in diameter, 2.1 μm in diameter, 2.2 μm in diameter, 2.3 μm in diameter, 2.4 μm in diameter, 2.5 μm in diameter, 2.6 μm in diameter, 2.7 μm in diameter, 2.8 μm in diameter, 2.9 μm in diameter, 3 μm in diameter, and values in between.

The compositions in embodiments are formulated in unit dosage form, each dosage containing a therapeutically effective amount of the active ingredients, for example in the dosage amounts disclosed below. The term “unit dosage form” may refer to a physically discrete unit suited as unitary dosages for the subject to be treated, each unit containing a predetermined quantity of active material calculated to produce the desired therapeutic effect(s), in association with a suitable pharmaceutical carrier, diluent, or excipient. Unit dosage forms are often used for ease of administration and uniformity of dosage. Unit dosage forms can contain a single or individual dose or unit, a sub-dose, or an appropriate fraction thereof (e.g., ¼, ⅓, ½ of a “full” dose), of the pharmaceutical composition administered. Unit dosage forms also may be subdivided (e.g., tablets may be scored) to permit fractional doses to be easily administered.

Unit dosage forms include capsules, troches, cachets, lozenges, tablets, ampules and vials, which may include a composition in a freeze-dried or lyophilized state; a sterile liquid carrier, for example, can be added prior to administration or delivery in vivo. Unit dosage forms also include ampules and vials with liquid compositions disposed therein. Unit dosage forms further include compounds for transdermal administration, such as “patches” that contact the epidermis (including the mucosa) of a subject for an extended or brief period of time.

In embodiments, the compositions of the invention are formulated in a pharmaceutically acceptable oral dosage form. Oral dosage forms include oral liquid dosage forms (such as tinctures, drops, emulsions, syrups, elixirs, suspensions, and solutions, and the like) and oral solid dosage forms. The disclosed pharmaceutical compositions also may be prepared as formulations suitable for intramuscular, subcutaneous, intraperitoneal, or intravenous injection, comprising physiologically acceptable sterile aqueous or non-aqueous solutions, dispersions, suspensions or emulsions, liposomes, and sterile powders for reconstitution into sterile injectable solutions or dispersions.

a. Oral Solid Dosage Forms

Oral solid dosage forms may include but are not limited to, lozenges, troches, tablets, capsules, caplets, powders, pellets, multiparticulates, beads, spheres, and/or any combinations thereof. Oral solid dosage forms may be formulated as immediate release, controlled release, sustained release, extended release, or modified release formulations.

In some embodiments, the oral solid dosage forms of the invention may be in the form of a tablet (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multi particulate dosage forms, pellets, granules, or an aerosol. In other embodiments, the pharmaceutical formulation is in the form of a powder. In still other embodiments, the pharmaceutical formulation is in the form of a tablet, including a fast-melt tablet. Additionally, pharmaceutical formulations of the invention may be administered as a single capsule or in multiple capsule dosage form. In embodiments, the pharmaceutical formulation is administered in two, three, four, or more capsules or tablets.

Oral solid dosage forms may contain pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof. Oral solid dosage forms can comprise one or more pharmaceutically acceptable additives such as a compatible carrier, complexing agent, ionic dispersion modulator, disintegrating agent, surfactant, lubricant, colorant, moistening agent, plasticizer, stabilizer, wetting agent, anti-foaming agent, alone or in combination, as well as supplementary active agent(s).

Supplementary active agents include preservatives, antioxidants, antimicrobial agents including biocides and biostats such as antibacterial, antiviral and antifungal agents. “Preservatives” include any substance useful in attenuating, inhibiting, or preventing microbial growth when added to a separate substance in which microorganisms are capable of establishing. Preservatives include antioxidants and antibacterial, antimicrobial, germicidal, or germistatic agents. Exemplary antioxidants include gluconolactone, benzalkonium chloride, benzoic acid or benzoates, such as sodium benzoate; vitamin A, vitamin C (ascorbic acid), vitamin E, tocopherols, alpha-lipoic acid, glutathione, vitamin E, superoxide dismutase, catalase, pNaKtide, Butylated hydroxytoluene, Butylated hydroxyanisole, tert-Butylhydroquinone, HP beta CD, resveratrol, retinol, coenzyme q10, niacinamide, polyphenols, flavonoids, beta-carotene, lutein, and lycopene. In embodiments, the preservatives are one or more of phenoxyethanol, capryl glycol, ethylhexylglycerin, and hexylene glycol. Preservatives may be included at concentrations effective to inhibit microbial growth, reduce ultraviolet light and/or oxygen-induced breakdown of composition components, or for like aims.

Using standard coating procedures, a film coating may be provided around the disclosed active agents (see Remington, supra). In embodiments, some or all of the disclosed active agents are coated. In embodiments, some or all of the disclosed active agents are microencapsulated. In embodiments, some or all of the disclosed active agents are amorphous material coated and/or microencapsulated with inert excipients. In embodiments, the disclosed active agents are not microencapsulated and are uncoated.

Suitable carriers for use in oral solid dosage forms include, but are not limited to, acacia, gelatin, colloidal silicon dioxide, calcium glycerophosphate, calcium lactate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose (HPMC), hydroxypropylmethylcellulose acetate stearate (HPMCAS), sucrose, microcrystalline cellulose, lactose, and mannitol.

Suitable filling agents for use in oral solid dosage forms include lactose, calcium carbonate, calcium phosphate, dibasic calcium phosphate, calcium sulfate, microcrystalline cellulose, cellulose powder, dextrose, dextrates, dextrose, dextran, starches, pregelatinized starch, HPMC, HPMCAS, HPMC phthalate, sucrose, xylitol, lactitol, mannitol, sorbitol, sodium chloride, and PEG.

Suitable disintegrants for use in oral solid dosage forms include those disclosed below for oral liquid aqueous suspensions and dispersions.

Suitable binders impart cohesiveness to solid oral dosage form formulations. For powder-filled capsules, they aid in plug formation that can be filled into soft or hard shell capsules. For tablets, they ensure that the tablet remains intact after compression and help assure blend uniformity prior to a compression or fill step. Materials suitable for use as binders in the solid dosage forms described herein include celluloses, microcrystalline dextrose, amylose, magnesium aluminum silicate, polysaccharide acids, bentonites, gelatin, polyvinylpyrrolidone/vinyl acetate copolymer, cross-povidone, povidone, starch, pregelatinized starch, tragacanth, dextrin, a sugar (e.g., sucrose, glucose, dextrose, molasses, mannitol, sorbitol, xylitol, lactose), a natural or synthetic gum (e.g., acacia, tragacanth, ghatti gum, mucilage of isapol husks), starch, PVP, larch arabogalactan, Veegum®, PEG, waxes, and sodium alginate.

In general, binder levels of between 20% to 70% are used in powder-filled gelatin capsule formulations. Binder usage level in tablet formulations is a function of whether direct compression, wet granulation, roller compaction, or usage of other excipients such as fillers which itself can act as moderate binders are used. Formulators skilled in the art can determine binder level for formulations, but binder usage of up to 70% in tablet formulations is common.

Some embodiments comprise lubricants or glidants. Suitable lubricants or glidants for use in oral solid dosage forms include, but are not limited to stearic acid, calcium hydroxide, talc, corn starch, sodium stearyl fumarate, alkali-metal and alkaline earth metal salts, stearic acid, sodium stearates, magnesium stearate, zinc stearate, waxes, Stearowet®, boric acid, sodium benzoate, sodium acetate, sodium chloride, leucine, PEG, methoxy-polyethylene glycol, propylene glycol, sodium oleate, glyceryl behenate, glyceryl palmitostearate, glyceryl benzoate, and magnesium or sodium lauryl sulfate.

Some embodiments comprise diluents. Suitable diluents for use in oral solid dosage forms include sugars, including lactose, sucrose, and dextrose; polysaccharides, including dextrates and maltodextrin; polyols, including mannitol, xylitol, and sorbitol; and cyclodextrins. Non-water-soluble diluents are compounds typically used in the formulation of pharmaceuticals, such as calcium phosphate, calcium sulfate, starches, modified starches and microcrystalline cellulose, and micro cellulose (e.g., having a density of about 0.45 g/cm3, e.g., Avicel, powdered cellulose), and talc.

Some embodiments comprise wetting agents. Suitable wetting agents for use in oral solid dosage forms include oleic acid, triethanolamine oleate, glyceryl monostearate, sorbitan monooleate, sorbitan monolaurate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, and vitamin E TPGS. Wetting agents, generally, include surfactants.

Some embodiments comprise surfactants. Suitable surfactants for use in the solid dosage forms described herein include docusate and its pharmaceutically acceptable salts, sodium lauryl sulfate, sorbitan monooleate, poly-oxyethylene sorbitan monooleate, polysorbates, poloxamers, bile salts, glyceryl monostearate, copolymers of ethylene oxide and propylene oxide, e.g., Pluronic® (BASF), and the like.

Some embodiments comprise suspending agents. Suitable suspending agents for use in oral solid dosage forms include, but are not limited to polyvinylpyrrolidone, PEG—having a molecular weight of between 300 to 6000, between 3350 to 4000, or 7000 to 18000; vinylpyrrolidone/vinyl acetate copolymer (S630), sodium alginate, gums, for example, gum tragacanth and gum acacia, guar gum, xanthans, including xanthan gum; sugars, celluloses, polysorbate-80, polyethoxylated sorbitan monolaurate, polyethoxylated sorbitan monolaurate, and povidone.

Some embodiments comprise antioxidants. Suitable antioxidants for use in oral solid dosage forms include gluconolactone, sodium benzoate, ascorbic acid, alpha-lipoic acid, glutathione, vitamin E, superoxide dismutase, catalase, pNaKtide, Butylated hydroxytoluene, Butylated hydroxyanisole, tert-Butylhydroquinone, HP beta CD, resveratrol, retinol, co-q10, niacinamide, polyphenols, flavonoids, beta-carotene, lutein, and lycopene.

In embodiments, the pharmaceutical composition may be an “immediate release formulation,” wherein a therapeutically effective amount of the pharmaceutical composition is administered to the subject in a way that facilitates rapid release. Immediate-release formulations may be prepared by combining a superdisintegrant such as croscarmellose sodium and different grades of microcrystalline cellulose in different ratios. In embodiments, to aid disintegration, sodium starch glycolate may be added.

In embodiments, where different agents included in the fixed-dose pharmaceutical composition of the invention are incompatible, cross-contamination can be avoided by incorporation of the agents in different layers in the oral dosage form with the inclusion of barrier layer(s) between the different layers, wherein the barrier layer(s) comprise inert and non-functional material(s).

The above-listed additives should be taken as merely exemplary types of additives that may be included in solid dosage forms of the invention. The amounts of such additives can be readily determined by one of skill, according to the particular properties desired.

Tablets of the invention can be prepared by methods well known in the art. Various methods for the preparation of the immediate release, modified release, controlled release, and extended-release dosage forms (e.g., as matrix tablets having one or more modified, controlled, or extended-release layers) and the vehicles therein are well known in the art. For example, a tablet may be made by compression or molding. Compressed tablets may be prepared by compressing, in a suitable machine, an active ingredient in a free-flowing form such as a powder or granules, optionally mixed with a binder, lubricant, inert diluent, preservative, surface-active or dispersing agent. Molded tablets may be produced by molding, in a suitable apparatus, a mixture of powdered compound moistened with an inert liquid diluent. The tablets may optionally be coated or scored and may be formulated so as to provide a slow or controlled release of the active ingredient therein. Generally recognized compendia of methods include: Remington (2020); Sheth et al. (1980), Compressed tablets, in Pharm. dosage forms, Vol. 1, Lieberman & Lachtman, eds., Dekker, NY.

In embodiments, solid dosage forms are prepared by mixing the disclosed active agents with one or more pharmaceutical excipients to form a “bulk blend” composition. The bulk blend composition is homogeneous, i.e., the active agents are dispersed evenly throughout so that the bulk blend may be readily subdivided into equally effective unit dosage forms, such as tablets, pills, and capsules. The individual unit dosages may also comprise film coatings, which disintegrate upon oral ingestion or upon contact with diluents. These formulations can be manufactured by conventional pharmaceutical techniques.

Conventional pharmaceutical techniques for preparation of solid dosage forms include, but are not limited to, the following methods, which may be used alone or in combination: (1) dry mixing, (2) direct compression, (3) milling, (4) dry or non-aqueous granulation, (5) wet granulation, or (6) fusion. (See, e.g., Lachman et al., Theory and Practice of Industrial Pharmacy (1986).) Other methods include spray drying, pan coating, melt granulation, granulation, fluidized bed spray drying or coating (e.g., Wurster coating), tangential coating, top spraying, tableting, and extruding.

Compressed tablets are solid dosage forms prepared by compacting the bulk blend. In embodiments, compressed tablets which are designed to dissolve in the mouth will comprise one or more flavoring agents. In embodiments, the compressed tablets will comprise a film surrounding the final compressed tablet. In embodiments, the film coating can provide a delayed release of the disclosed active agents. In embodiments, the film coating aids in patient compliance (e.g., flavor or sweetener coatings).

In embodiments, a capsule may be prepared by placing the bulk blend inside of a capsule, such as a soft gelatin capsule, a standard gelatin capsule, or a non-gelatin capsule such as a capsule comprising HPMC. The bulk blend also may be placed in a sprinkle capsule, wherein the capsule may be swallowed whole or the capsule may be opened and the contents sprinkled on food prior to eating. In embodiments, the therapeutic dose is split into multiple capsules. In embodiments, the entire dose of the active agent(s) of the invention is delivered in a capsule form. In embodiments the capsule is a size 000, size 00, or size 0 soft gelatin capsule. In embodiments, the capsule is a size 1, size 2, size 3, size 4, or size 5 soft gelatin capsule. In embodiments, the capsule is a hard gelatin capsule of equivalent size.

In embodiments, the formulations of the invention are fixed-dose disclosed pharmaceutical compositions and at least one other pharmacological agent. Fixed-dose combination formulations may contain therapeutically efficacious fixed-dose combinations of formulations of the disclosed active agents and other pharmacological agents in the form of a single-layer monolithic tablet or multi-layered monolithic tablet or in the form of a core tablet-in-tablet or multi-layered, multi-disk tablet or beads inside a capsule or tablets inside a capsule.

Depending on the desired release profile, oral solid dosage forms may be prepared as immediate release formulations, or as modified release formulations, such as controlled release, extended release, sustained release, or delayed release. In embodiments, the release profile is determined by obtaining objective measurements from a patient, the objective measurements including but are not limited to, weight, body temperature, heart rate, respiratory rate, blood oxygenation, blood pressure (BP) and its variables, including, but not limited to: systolic (SBP), diastolic (DBP), mean arterial (MAP), and pulse (PP); Continuous Beat-by-Beat Blood Pressure (CNIBP), measurements from an electrocardiogram (ECG), including RR interval or its variability, QT interval or its variability, heart rate variability (HRV) (or measured by devices other than an ECG); hemodynamic response (HR), and levels of glucose, cortisol, serotonin, dopamine, cholesterol; and numerous more, as readily appreciated by those of skill.

In embodiments of modified release formulations, the half-life compared to the half-life of an immediate release formulation is greater by at least 5%, at least 10%, at least 15%, at least 20%, at least 25%, at least 30%, at least 35%, at least 50%, at least 75%, at least 100%, at least 120%, at least 150%, at least 175%, at least 200%, and values in between. In embodiments of modified release formulations, the formulations are designed to result in a comparable “area under the curve,” or AUC₀₋₂₄, and a similar safety and efficacy profile, but having a delayed time to maximum concentration (tₘₐₓ) of at least 5%, at least 10%, at least 15%, at least 20%, at least 25%, at least 30%, at least 35%, at least 50%, at least 75%, at least 100%, at least 120%, at least 150%, at least 175%, at least 200%, and values in between. In some preferred embodiments, a formulation is designed to be a product with a specific time course based on an optimum “therapeutic window,” such as less than 30 minutes, 30 minutes, 45 minutes, 60 minutes, 90 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, and lengths of time in between, as understood by one of skill.

In embodiments, oral solid dosage forms are formulated as a delayed release dosage form by utilizing an enteric coating to affect release in the small intestine of the gastrointestinal tract. An enteric-coated oral dosage form may be a compressed or molded or extruded tablet/mold (coated or uncoated) containing granules, powder, pellets, beads or particles of the active ingredient and/or other composition components, which are themselves coated or uncoated. The enteric-coated oral dosage form may also be a capsule (coated or uncoated) containing pellets, beads or granules of the solid carrier or the composition, which are themselves coated or uncoated.

In embodiments, enteric coatings may be used to prepare other controlled release dosage forms, including but not limited to extended release and pulsatile release dosage forms. Pulsatile release dosage forms may be formulated using techniques known in the art, such as those described in U.S. Pat. Nos. 5,011,692, 5,017,381, 5,229,135, and 5,840,329. Other suitable dosage forms are described in U.S. Pat. Nos. 4,871,549, 5,260,068, 5,260,069, 5,508,040, 5,567,441 and 5,837,284.

Using such means, a single unit dosage form can provide both a first and a second dosage amount in the single form (e.g., the first dosage amount in an immediate release form, and the second dosage amount in a delayed release form).

In embodiments, gastroretentive sustained release tablets are formulated by using a combination of hydrophilic polymer (e.g., hydroxypropyl methylcellulose), together with at least one swelling agent (e.g., crospovidone, sodium starch glycolate, and croscarmellose sodium), and an effervescent substance (e.g., sodium bicarbonate). Using known methods, gastroretentive tablets can be formulated to prolong the gastric emptying time and extend the mean residence time (MRT) in the stomach for optimal drug release and absorption (see, e.g., Arza et al. Formulation and evaluation of swellable and floating gastroretentive ciprofloxacin hydrochloride tablets, AAPS PharmSciTech., 10(1):220-226 (2009)).

Coatings for providing a controlled, delayed, or extended release may be applied to the disclosed pharmaceutical compositions or to a core containing the compositions. The coating may comprise a pharmaceutically acceptable ingredient in an amount sufficient to provide an delayed release from, for example, 1 hour to 7 hours following ingestion before release of the compositions. Suitable coatings include one or more differentially degradable coatings including pH-sensitive coatings (enteric coatings), or non-enteric coatings having variable thickness to provide differential release of the active agents.

It will be appreciated that the above discussion regarding release systems is meant to be exemplary, and should not be construed as limiting. Many other types of modified release systems are known to those of skill and are suitable for the formulations described herein. Non-limiting examples of such delivery systems include both polymer- and nonpolymer-based systems, silastic systems, peptide-based systems, wax coatings, bioerodible dosage forms, and compressed tablets using conventional binders (see e.g., Liberman et al. Pharmaceutical Dosage Forms, 2 Ed., Vol. 1, pp. 209-214 (1990); Singh et al. Encyclopedia of Pharmaceutical Technology, 2nd Ed., pp. 751-753 (2002); U.S. Pat. Nos. 4,327,725; 4,624,848; 4,968,509; 5,461,140; 5,456,923; 5,516,527; 5,622,721; 5,686,105; 5,700,410; 5,977,175; 6,465,014; and 6,932,983.)

b. Oral Liquid Dosage Forms

Oral liquid dosage forms include tinctures, drops, emulsions, syrups, elixirs, suspensions, and solutions, and the like. These oral liquid dosage forms may be formulated with any pharmaceutically acceptable excipient known to those of skill in the art for the preparation of liquid dosage forms, and with solvents, diluents, carriers, excipients, and the like chosen as appropriate to the solubility and other properties of the active agents and other ingredients. Solvents may be, for example, water, glycerin, simple syrup, alcohol, medium chain triglycerides (MCT), and combinations thereof.

Oral liquid dosage forms are advantageous in treating geriatric and pediatric populations, as the liquid formulation is much easier to swallow. The liquid formulation is also absorbed more rapidly than a solid dosage form, as it need not be dissolved by the digestive system before it is available for absorption.

Oral liquid dosage forms are either monophasic or biphasic, the former being a substantially homogenous solution dissolved in water or non-aqueous solvent, while the latter may refer to oral liquid dosage forms in which the active ingredients do not fully dissolve in common solvents. Thus, over time, the solid particles (i.e., the active ingredients) within the oral liquid dosage form may form a precipitate at the bottom of the container-requiring vigorous shaking to redisperse the active ingredients.

Examples of monophasic liquid forms include syrups, linctuses, spirits/essences, elixirs, and fluid extracts. Examples of biphasic liquid forms include oral suspensions, oral emulsions, and mixtures.

Liquid dosage forms for oral administration may be prepared as liquid suspensions or solutions using a sterile liquid, such as but not limited to, an oil, water, an alcohol, combinations of pharmaceutically suitable surfactants, suspending agents, and emulsifying agents (herein “emulsifying wax.”) Liquid formulations also may be prepared as single dose or multi-dose beverages. Suspensions may include oils. Such oils include peanut oil, sesame oil, cottonseed oil, corn oil, and olive oil. Suitable oils also include carrier oils such as MCT and long chain triglyceride (LOT) oils. Suspension preparation may also contain esters of fatty acids such as ethyl oleate, isopropyl myristate, fatty acid glycerides, and acetylated fatty acid glycerides. Suspension formulations include alcohols, such as ethanol, isopropyl alcohol, hexadecyl alcohol; glycerol, and propylene glycol. Ethers, such as polyethylene glycol; petroleum hydrocarbons, such as mineral oil and petrolatum; and water may also be used in suspension formulations. Suspensions include an aqueous liquid or a non-aqueous liquid, an oil-in-water liquid emulsion, or a water-in-oil emulsion.

In embodiments, formulations are provided comprising the compositions of the invention and at least one dispersing agent or suspending agent for oral administration to a subject. The formulation may be a powder and/or granules for suspension wherein, upon admixture with water, a substantially uniform suspension forms. The aqueous dispersion may comprise amorphous and non-amorphous particles including multiple effective particle sizes to facilitate drug absorption in a controlled manner over time.

Dosage forms for oral administration may be aqueous suspensions such as pharmaceutically acceptable aqueous oral dispersions, emulsions, solutions, and syrups. See Singh et al., Encyclopedia of Pharm. Tech., 2nd Ed., 754-757 (2002). In addition to the disclosed active agents, the liquid dosage forms may comprise additives, such as one or more (a) disintegrating agents, (b) dispersing agents, (c) wetting agents, (d) preservatives, (e) viscosity enhancing agents, (f) sweetening agents, or (g) flavoring agents.

In embodiments, effervescent powders containing the compositions of the invention may be prepared. Effervescent salts are used to disperse medicines in water for oral administration. Effervescent salts also may be packaged as single dose or multi-dose drink mixes, alone or in combination with other ingredients, such as vitamins or electrolytes. Effervescent salts are granules or coarse powders containing a medicinal agent in a dry mixture, usually composed of sodium bicarbonate and sodium carbonate, citric acid, and/or tartaric acid. When salts of the invention are added to water, the acids and the base react to liberate carbon dioxide gas, thereby causing “effervescence.” Any acid-base combination that results in the liberation of carbon dioxide may be used, as long as the ingredients are suitable for pharmaceutical use, and result in a pH of 6.0 or higher.

In embodiments, disintegrating agents are included in the oral liquid dosage formulation. Non-limiting examples of disintegrating agents for use in the aqueous suspensions and dispersions include at least one starch, e.g., a natural starch such as corn starch or potato starch, a pregelatinized starch, or sodium starch glycolate; a cross-linked starch such as sodium starch glycolate; at least one cellulose, such as a wood product, microcrystalline cellulose, methylcellulose, croscarmellose, or a cross-linked cellulose, such as cross-linked sodium carboxymethylcellulose, cross-linked carboxymethylcellulose, or cross-linked croscarmellose; a cross-linked polymer such as crosspovidone; a cross-linked polyvinylpyrrolidone; alginate, such as alginic acid or a salt of alginic acid such as sodium alginate; a clay; a gum such as agar, guar, locust bean, Karaya, pectin, or tragacanth; sodium starch glycolate; bentonite; a natural sponge; a surfactant; a resin such as a cation-exchange resin; citrus pulp; and sodium lauryl sulfate.

In embodiments, dispersing agents are included in the oral liquid dosage formulation. Examples of dispersing agents suitable for the aqueous suspensions and dispersions include hydrophilic polymers, electrolytes, Tween® 60 or 80, polyethylene glycol (PEG), polyvinylpyrrolidone (PVP), carbohydrate-based dispersing agents, noncrystalline cellulose, magnesium aluminum silicate, triethanolamine, polyvinyl alcohol (PVA), polyvinylpyrrolidone/vinyl acetate copolymer, poloxamers, and poloxamines.

In embodiments, wetting agents are included in the oral liquid dosage formulation. Non-limiting examples of wetting agents (including surfactants) suitable for the aqueous suspensions and dispersions include acetyl alcohol, glycerol monostearate, polyoxyethylene sorbitan fatty acid esters, PEG, oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, and phosphatidylcholine.

In embodiments, preservatives are included in the oral liquid dosage formulation. Non-limiting examples of preservatives suitable for aqueous suspensions or dispersions include potassium sorbate, parabens (e.g., methylparaben and propylparaben) and their salts, benzoic acid and its salts, other esters of para hydroxybenzoic acid such as butylparaben, alcohols such as ethyl alcohol or benzyl alcohol, phenolic compounds such as phenol, and quaternary compounds such as benzalkonium chloride. Preservatives, as used herein, are incorporated into the dosage form at a concentration sufficient to inhibit microbial growth.

In embodiments, viscosity enhancing agents are included in the oral liquid dosage formulation. Non-limiting examples of viscosity enhancing agents suitable for aqueous suspensions or dispersions include methyl cellulose, xanthan gum, carboxymethylcellulose, hydroxypropyl cellulose, hydroxypropylmethyl cellulose, Plasdone® S-630, carbomer, polyvinyl alcohol, alginates, acacia, chitosans, and combinations thereof. The concentration of the viscosity-enhancing agent will depend upon the agent selected and the viscosity desired.

In addition to the additives listed above, liquid formulations, in embodiments, also may comprise inert diluents commonly used in the art such as water or other solvents, solubilizing agents, emulsifiers, flavoring agents, and/or sweeteners. Co-solvents and adjuvants also may be added to a formulation. Non-limiting examples of co-solvents contain hydroxyl groups or other polar groups, for example, alcohols, glycols, glycerol, polyoxyethylene alcohols, and polyoxyethylene fatty acid esters. Adjuvants include surfactants such as soy lecithin and oleic acid, sorbitan esters such as sorbitan trioleate, and PVP.

c. Transdermal Delivery

In some embodiments, disclosed pharmaceutical compositions may be administered via transdermal delivery. Generally speaking, transdermal delivery involves contacting the formulations of the invention with a subject's skin under conditions effective for the active agent(s) to penetrate the skin and cause an effect. In embodiments, the formulations of the invention may be administered in and/or around the abdomen, back, chest, legs, arms, scalp or other suitable skin surface, by applying the formulation directly to the skin or by using delivery devices such as bandages, patches, and/or the like. In preferred embodiments, as discussed previously, the formulations of the invention are administered directly to the skin as a hydrogel formulation. In alternative embodiments, the formulations may be prepared as ointments, creams, suspensions, lotions, pastes, gels, sprays, foams, oils, and the like, and any combination thereof.

A formulation also can be applied directly to the skin and then covered to minimize the likelihood of its being disturbed. Alternatively, a formulation can be coated on the surface of a bandage, gauze, etc., which can then be applied to the skin of a subject such that the formulation is in direct contact with the subject's skin. Alternatively, a formulation can be delivered transdermally to a subject by preparing the formulation directly into a bandage, pad, film, or other type of patch which can be applied to a subject's skin.

After application of the formulation to a subject's skin, essentially no limitations exist as to the length of time the formulation can remain in contact with the subject's skin. Since the amount of active agent(s) in the formulation will decrease as it is absorbed into a subject's skin, the formulation can be removed when the amount of active agent(s) remaining in the formulation decreases to an amount that is no longer effective to the subject. It is to be understood that the amount of active agent(s) initially carried in the formulation will affect the length of time the formulation will be effective once the formulation is applied to the subject's skin. However, the formulation may optionally be left on longer than, or removed sooner than, the length of time that is necessary or recommended for complete diffusion of the active agent into the skin.

An exemplary transdermal delivery device is the transdermal “patch,” which itself contains the pharmaceutical compositions. Such transdermal patches may be used to provide continuous or discontinuous infusion of the disclosed compounds in controlled amounts. Such patches may be constructed for continuous, gradual, pulsatile, or on demand delivery of pharmaceutical agents. A “patch” within the meaning of the invention may be simply a medicated adhesive patch, i.e., a patch impregnated with a disclosed composition for application onto the skin. Thus, a patch may be a single-layer or multi-layer drug-in-adhesive patch, wherein the one or more adhesive layers also contain the active agents.

A patch may also be a “matrix” (or “monolithic”) patch, wherein the adhesive layer surrounds and overlays the drug layer (wherein a solution or suspension of the active agents is in a semisolid matrix). A “reservoir” patch may also be used, comprising a drug layer, typically as a solution or suspension of the active agents in a liquid compartment (i.e., the reservoir), separate from an adhesive layer. For example, the reservoir may be totally encapsulated in a shallow compartment molded from a drug-impermeable metallic plastic laminate, with a rate-controlling membrane made of vinyl acetate or a like polymer on one surface. A patch also may be part of a delivery system, for instance used with an electronic device communicatively coupled to the mobile device of a user, and coupled with a mobile application (e.g., to control the delivery rate from the reservoir, and optionally to provide information about delivery back to the application or user). Various transdermal patch technologies may be accordingly utilized.

In embodiments, a transdermal patch technology comprises a self-contained module including a built-in battery that produces a low-level electric current to heat the skin and deliver a prescribed dose of a disclosed composition, wherein a therapeutically effective amount of the composition crosses the skin and enters the underlying tissue, so as to produce a biologic effect. An exemplary device comprises an adhesive layer, a protective film, a drug-containing reservoir (for the disclosed pharmaceutical compositions), a heating coil, a battery, a hardware board, optionally all within a device holder, and optionally, functionally coupled to a device which is able to control drug delivery (e.g., a mobile device such as a smartphone) using a downloadable application. Such devices may, for instance, additionally shut off drug delivery automatically when a prescribed dose has been administered, or may shut off automatically upon reaching a certain temperature or defined time. Such transdermal devices may be reusable or disposable.

In embodiments, where a disclosed composition is administered transdermally, it will penetrate the skin, as will be determined based on measurements known to those of skill, to a depth of at least 0.25 mm, at least 0.5 mm, at least 0.75 mm, at least 1 mm, at least 1.25 mm, at least 1.5 mm, at least 1.75 mm, at least 2 mm, at least 2.25 mm, at least 2.5 mm, at least 2.75 mm, at least 3 mm, at least 3.25 mm, at least 3.5 mm, at least 3.75 mm, at least 4 mm, at least 4.25 mm, at least 4.5 mm, at least 4.75 mm, at least 5 mm, and values in between.

In embodiments, where a disclosed composition is administered transdermally, it will penetrate the skin, as will be determined based on measurements known to those of skill, to a depth of at least the keratotic layer, at least the stratum corneum, at least the stratum lucidum, at least the stratum granulosum, at least the stratum spinosum, or at least the stratum basale, including penetrating all layers of the epidermis to the dermis, and in embodiments including penetrating all layers of the dermis to the hypodermis, e.g., to the subcutaneous tissue or superficial fascia underneath.

d. Injection

In some embodiments, the compositions of the invention may also be prepared as suspension formulations designed for extended release via subcutaneous, intravenous, intra-arterial, intraperitoneal, intraosseous, intramuscular, intrathecal, or intracerebroventricular, injection. Such formulations avoid first-pass metabolism, and lower dosages of the active agents will be necessary to maintain equivalent plasma levels when compared to oral formulations. In such formulations, the mean particle size of the active agents and the range of total particle sizes can be used to control the release of those agents by controlling the rate of dissolution in fat or muscle. For example, prolonged drug absorption of an injectable form may be facilitated by use of agents delaying absorption, non-limiting examples of which include aluminum monostearate and gelatin. The compositions also may be prepared for microinjection or injection cannula.

Injection formulations may be prepared by dissolving, suspending, or emulsifying the active agent(s) in an aqueous or nonaqueous solvent, non-limiting examples of which include oils, such as vegetable oil, synthetic aliphatic acid glycerides, and esters of higher aliphatic acids or propylene glycol; and may also contain additives such as solubilizers, stabilizers, and suspending, preserving, wetting, emulsifying, dispensing, and isotonic agents. Preserving agents useful for the invention include, but are not limited to, antibacterial and antifungal agents, such as parabens, benzoic acid, benzyl alcohol, chlorobutanol, phenol, and sorbic acid. Isotonic agents useful for the invention include, but are not limited to, sugars and sodium chloride.

In embodiments, a composition is formulated in an aqueous solution, including but not limited to physiologically compatible buffers such as Hanks's solution, Ringer's solution, or a physiological saline buffer. In embodiments, injection formulations are presented in unit dosage form, including but not limited to ampoules or multi-dose containers, with or without an additive as described above. In embodiments, the compositions may be suspensions, solutions, or emulsions in oily or aqueous vehicles, and may contain formulary agents, non-limiting examples of which include suspending, stabilizing, and/or dispersing agents.

e. Nanostructured Formulations

In some embodiments, the pharmaceutical compositions disclosed herein are prepared for administration as a nanostructured formulation such as a nanoemulsion, a nanocapsule, a nanoparticle conjugate, or a nano-encapsulated oral or nasal spray. Preparations of the compositions of the invention as certain nanostructured formulations may be done by reference to the general knowledge of the art. (See, e.g., Jaiswal et al., Nanoemulsion: an advanced mode of drug delivery system, Biotech 3(5):123-27 (2015).)

In embodiments, lipid-based nanoparticles (LBNPs) such as liposomes, solid lipid nanoparticles (SLN), and nanostructured lipid carriers (NLC) may be used to transport both hydrophobic and hydrophilic molecules, and may be formulated to display very low or no toxicity, and increase the time of drug action by means of prolonged half-life and controlled release of active agents. Lipid nanosystems also may include chemical modifications to avoid immune system detection (e.g., gangliosides or PEG) or to improve solubility of active agents. In addition, such nanosystems may be prepared in formulations sensitive to pH so as to promote drug release in an acid environment.

f. Topical Application

In some embodiments, disclosed pharmaceutical compositions may be formulated into a topical dosage form. Non-limiting examples of topical dosage forms include aerosols, emulsions, sprays, ointments, salves, gels, pastes, lotions, liniments, oils, and creams. These are all examples of epicutaneous applications—meaning they are administered directly to the skin—however, other forms of topical administration known in the art, including, for example, contrast media for imaging of the bowel, eye drops (onto the conjunctiva), e.g., antibiotics for conjunctivitis, ear drops, such as antibiotics and corticosteroids for otitis externa, and those that diffuse through mucous membranes in the body.

For all such formulations, penetrants and carriers may be included in the pharmaceutical composition. Topical formulations are similar to transdermal applications in that they are also applied directly to the patient's skin, although such formulations may be limited to localized administration, lacking the systemic release characterized by transdermal applications, and therefore in embodiments preferably applied directly where (local) action is desired.

g. Additional Dosage Forms

Some embodiments include suitable aqueous and non-aqueous carriers, diluents, solvents, or vehicles. Non-limiting examples of suitable aqueous and non-aqueous carriers, diluents, solvents, or vehicles include water, ethanol, polyols, suitable mixtures thereof, vegetable oils, and injectable organic esters such as ethyl oleate. Additionally, the compositions of the invention may be dissolved at concentrations of >1 mg/ml using water-soluble beta cyclodextrins (e.g., beta-sulfobutyl-cyclodextrin and 2-hydroxypropyl-betacyclodextrin). Proper fluidity may be maintained by the use of a coating such as a lecithin, by the maintenance of the required particle size in the case of dispersions, and with surfactants.

In embodiments, the disclosed compositions are provided as oral solid/oral liquid dosage forms, administered sublingually, buccally, topically, rectally, vaginally, ocularly, optically, nasally, cutaneously, topically, and transdermally; or as intravenous, intra-arterial, intraperitoneal, intraosseous, intramuscular, intrathecal, intracerebroventricular, and subcutaneous injection, wherein such injections comprise physiologically acceptable sterile aqueous or non-aqueous solutions, dispersions, suspensions or emulsions, liposomes, and sterile powders for reconstitution into sterile injectable solutions or dispersions.

### I. Dosage Methods

In some embodiments, the disclosed active agents are administered to a patient as a “combination drug” as part of “combination therapy,” wherein the individual active agents are administered to the patient separately, as would be appreciated by those of skill. A “combination drug” includes a single dosage form of a combination prepared by a compounding pharmacy, rather than manufactured together (a “fixed-dose combination”), wherein each single dosage form contains at least one active agent. The term also may refer to a “co-packaged drug,” i.e., a drug approved for sale as a single “product,” but where the product is a blister pack or other “pharmaceutical kit” containing the different drugs in separate dosage forms for co-administration. A “therapeutic combination” includes combination drugs and co-packaged drugs, as well as fixed-dose combinations, and further includes combinations of two or more separate pharmaceutical compositions administered separately.

Herein, “combination” therapy may refer to therapy in which the “combination drugs” are administered to a patient separately. This may include, but is not limited to, the combination drugs being administered sequentially, i.e., one combination drug is administered immediately following administering a separate combination drug; and the combination drugs being administered with time elapsing between the administrations. In embodiments, where the combination drugs are administered with time elapsing between the administrations, the elapsed time may be between 5 minutes to 30 minutes. In embodiments, where the combination drugs are administered with time elapsing between the administrations, the elapsed time may be between 10 minutes to 60 minutes. In embodiments, where the combination drugs are administered with time elapsing between the administrations, the elapsed time may be between 30 minutes to 180 minutes. In embodiments, where the combination drugs are administered with time elapsing between the administrations, the elapsed time may be between 45 minutes to 360 minutes. In embodiments, where the combination drugs are administered with time elapsing between the administrations, the elapsed time may be more than 360 minutes, such as 8 hours, 12 hours, 16 hours, 20 hours, 24 hours, 36 hours, 48 hours, 72 hours, 5 days, 7 days, 10 days, 14 days, 21 days, 30 days, and any such durations in between.

Herein, “sequential” administration, in which administration of the second combination drug “immediately” follows administration of the first, “immediate” may refer to administration of the second combination drug within a short period of time, such as within 5 minutes of the administration of the first combination drug, or within 4, within 3, within 2, within 1 minute, or lesser periods.

The time elapsed between doses in combination therapy should not be confused with the frequency at which dosing occurs. As will be appreciated by one of skill, the frequency of dosing, whether administered via combination therapy or as a fixed-dose combination, would depend on the disease and symptoms being treated. As a non-limiting example, it may be desirable in embodiments to administer combination drugs as part of combination therapy twice per day or three times per day; in embodiments, one of the combination drugs may be administered twice per day, or three times per day, while the other combination drug is administered one time per day, or twice per day. The frequency of dosing and duration of elapsed time between administrations of separate combination drugs in combination therapy will be readily understood by one of skill.

In embodiments, the separate application may comprise separate oral administrations, separate sublingual administrations, separate buccal administrations, separate intravenous injections, separate intra-arterial injections, separate intraperitoneal injections, separate intraosseous injections, separate intramuscular injections, separate intrathecal injections, separate intracerebroventricular injections, separate rectal administrations, separate vaginal administrations, separate ocular administrations, separate nasal administrations, separate cutaneous administrations, separate topical administrations, separate otic administrations, separate transdermal administrations, or a combination thereof, as would be apparent to one of skill depending on the desired therapeutic effect. For example, it may be advantageous to utilize a method with rapid absorption, non-limiting examples of which include intravenous injection and liquid oral dosage forms, in combination with a means for a slower release profile, like that of transdermal application. Further, as would be readily appreciated by one of skill, the order of the combination therapy is dependent on desired therapeutic effects.

In embodiments, the second active agent is administered prior to the first active agent. In embodiments, the first active agent is administered prior to, and followed by, the second active agent. The order of application will be determined by the properties of the active agents and their methods of administration. As a non-limiting example, if it is beneficial to use transdermal release for the second active agent—to utilize a slower release profile and/or bypass the first-pass metabolism—and intravenous injection for the first active agent, to elicit a more rapid release, but desirable to have both active agents elicit their respective therapeutic effects simultaneously, it may be necessary to first apply the transdermal solution containing the second active agent before later injecting the first active agent, as understood by one of skill.

In embodiments, the separate application may comprise administration of a first active agent via oral administration means, non-limiting examples of which include oral solid and oral liquid dosage forms, and administration of a second active agent via any of: oral administration means, non-limiting examples of which include oral solid and liquid dosage forms; sublingual, buccal, intravenous, intra-arterial, intraperitoneal, intraosseous, intramuscular, intrathecal, intracerebroventricular, rectal, vaginal, ocular, nasal, cutaneous, topical, otic, subcutaneous, or transdermal administration means.

In embodiments, the separate application may comprise administration of a first active agent via sublingual administration, and administration of a second active agent via any of: oral administration means, non-limiting examples of which include oral solid and liquid dosage forms; sublingual, buccal, intravenous, intra-arterial, intraperitoneal, intraosseous, intramuscular, intrathecal, intracerebroventricular, rectal, vaginal, ocular, nasal, cutaneous, topical, otic, subcutaneous, or transdermal administration means.

In embodiments, the separate application may comprise administration of a first active agent via buccal administration, and administration of a second active agent via any of: oral administration means, non-limiting examples of which include oral solid and liquid dosage forms; sublingual, buccal, intravenous, intra-arterial, intraperitoneal, intraosseous, intramuscular, intrathecal, intracerebroventricular, rectal, vaginal, ocular, nasal, cutaneous, topical, otic, subcutaneous, or transdermal administration means.

In embodiments, the separate application may comprise administration of a first active agent via intravenous injection, and administration of a second active agent via any of: oral administration means, non-limiting examples of which include oral solid and liquid dosage forms; sublingual, buccal, intravenous, intra-arterial, intraperitoneal, intraosseous, intramuscular, intrathecal, intracerebroventricular, rectal, vaginal, ocular, nasal, cutaneous, topical, otic, subcutaneous, or transdermal administration means.

In embodiments, the separate application may comprise administration of a first active agent via intra-arterial injection, and administration of a second active agent via any of: oral administration means, non-limiting examples of which include oral solid and liquid dosage forms; sublingual, buccal, intravenous, intra-arterial, intraperitoneal, intraosseous, intramuscular, intrathecal, intracerebroventricular, rectal, vaginal, ocular, nasal, cutaneous, topical, otic, subcutaneous, or transdermal administration means.

In embodiments, the separate application may comprise administration of a first active agent via intraperitoneal injection, and administration of a second active agent via any of: oral administration means, non-limiting examples of which include oral solid and liquid dosage forms; sublingual, buccal, intravenous, intra-arterial, intraperitoneal, intraosseous, intramuscular, intrathecal, intracerebroventricular, rectal, vaginal, ocular, nasal, cutaneous, topical, otic, subcutaneous, or transdermal administration means.

In embodiments, the separate application may comprise administration of a first active agent via intraosseous injection, and administration of a second active agent via any of: oral administration means, non-limiting examples of which include oral solid and liquid dosage forms; sublingual, buccal, intravenous, intra-arterial, intraperitoneal, intraosseous, intramuscular, intrathecal, intracerebroventricular, rectal, vaginal, ocular, nasal, cutaneous, topical, otic, subcutaneous, or transdermal administration means.

In embodiments, the separate application may comprise administration of a first active agent via intramuscular injection, and administration of a second active agent via any of: oral administration means, non-limiting examples of which include oral solid and liquid dosage forms; sublingual, buccal, intravenous, intra-arterial, intraperitoneal, intraosseous, intramuscular, intrathecal, intracerebroventricular, rectal, vaginal, ocular, nasal, cutaneous, topical, otic, subcutaneous, or transdermal administration means.

In embodiments, the separate application may comprise administration of a first active agent via intrathecal injection, and administration of a second active agent via any of: oral administration means, non-limiting examples of which include oral solid and liquid dosage forms; sublingual, buccal, intravenous, intra-arterial, intraperitoneal, intraosseous, intramuscular, intrathecal, intracerebroventricular, rectal, vaginal, ocular, nasal, cutaneous, topical, otic, subcutaneous, or transdermal administration means.

In embodiments, the separate application may comprise administration of a first active agent via intracerebroventricular injection, and administration of a second active agent via any of: oral administration means, non-limiting examples of which include oral solid and liquid dosage forms; sublingual, buccal, intravenous, intra-arterial, intraperitoneal, intraosseous, intramuscular, intrathecal, intracerebroventricular, rectal, vaginal, ocular, nasal, cutaneous, topical, otic, subcutaneous, or transdermal administration means.

In embodiments, the separate application may comprise administration of a first active agent via rectal administration, and administration of a second active agent via any of: oral administration means, non-limiting examples of which include oral solid and liquid dosage forms; sublingual, buccal, intravenous, intra-arterial, intraperitoneal, intraosseous, intramuscular, intrathecal, intracerebroventricular, rectal, vaginal, ocular, nasal, cutaneous, topical, otic, subcutaneous, or transdermal administration means.

In embodiments, the separate application may comprise administration of a first active agent via vaginal administration, and administration of a second active agent via any of: oral administration means, non-limiting examples of which include oral solid and liquid dosage forms; sublingual, buccal, intravenous, intra-arterial, intraperitoneal, intraosseous, intramuscular, intrathecal, intracerebroventricular, rectal, vaginal, ocular, nasal, cutaneous, topical, otic, subcutaneous, or transdermal administration means.

In embodiments, the separate application may comprise administration of a first active agent via ocular administration, and administration of a second active agent via any of: oral administration means, non-limiting examples of which include oral solid and liquid dosage forms; sublingual, buccal, intravenous, intra-arterial, intraperitoneal, intraosseous, intramuscular, intrathecal, intracerebroventricular, rectal, vaginal, ocular, nasal, cutaneous, topical, otic, subcutaneous, or transdermal administration means.

In embodiments, the separate application may comprise administration of a first active agent via nasal administration, and administration of a second active agent via any of: oral administration means, non-limiting examples of which include oral solid and liquid dosage forms; sublingual, buccal, intravenous, intra-arterial, intraperitoneal, intraosseous, intramuscular, intrathecal, intracerebroventricular, rectal, vaginal, ocular, nasal, cutaneous, topical, otic, subcutaneous, or transdermal administration means.

In embodiments, the separate application may comprise administration of a first active agent via cutaneous administration, and administration of a second active agent via any of: oral administration means, non-limiting examples of which include oral solid and liquid dosage forms; sublingual, buccal, intravenous, intra-arterial, intraperitoneal, intraosseous, intramuscular, intrathecal, intracerebroventricular, rectal, vaginal, ocular, nasal, cutaneous, topical, otic, subcutaneous, or transdermal administration means.

In embodiments, the separate application may comprise administration of a first active agent via topical administration, and administration of a second active agent via any of: oral administration means, non-limiting examples of which include oral solid and liquid dosage forms; sublingual, buccal, intravenous, intra-arterial, intraperitoneal, intraosseous, intramuscular, intrathecal, intracerebroventricular, rectal, vaginal, ocular, nasal, cutaneous, topical, otic, subcutaneous, or transdermal administration means.

In embodiments, the separate application may comprise administration of a first active agent via otic administration, and administration of a second active agent via any of: oral administration means, non-limiting examples of which include oral solid and liquid dosage forms; sublingual, buccal, intravenous, intra-arterial, intraperitoneal, intraosseous, intramuscular, intrathecal, intracerebroventricular, rectal, vaginal, ocular, nasal, cutaneous, topical, otic, subcutaneous, or transdermal administration means.

In embodiments, the separate application may comprise administration of a first active agent via subcutaneous administration, and administration of a second active agent via any of: oral administration means, non-limiting examples of which include oral solid and liquid dosage forms; sublingual, buccal, intravenous, intra-arterial, intraperitoneal, intraosseous, intramuscular, intrathecal, intracerebroventricular, rectal, vaginal, ocular, nasal, cutaneous, topical, otic, subcutaneous, or transdermal administration means.

In embodiments, the separate application may comprise administration of a first active agent via transdermal administration, and administration of a second active agent via any of: oral administration means, non-limiting examples of which include oral solid and liquid dosage forms; sublingual, buccal, intravenous, intra-arterial, intraperitoneal, intraosseous, intramuscular, intrathecal, intracerebroventricular, rectal, vaginal, ocular, nasal, cutaneous, topical, otic, subcutaneous, or transdermal administration means.

In embodiments, the separate application may comprise administration of a first active agent via an administration method known to be effective by one of skill, and administration of a second active agent via an administration method known to be effective by one of skill.

### J. Methods of Administration

As mentioned above, a disclosed entactogenic combination may be administered via enteral or parenteral means, wherein enteral means includes, but is not limited to, oral solid and oral liquid dosage forms, sublingual, rectal, and buccal administration; and parenteral administration means includes, but is not limited to, intravenous, intra-arterial, intraperitoneal, intraosseous, intramuscular, intrathecal, intracerebroventricular, vaginal, ocular, nasal, cutaneous, topical, otic, transdermal, and subcutaneous administration; in addition to other equivalent means known to those of skill.

The term “administering” or “administration” broadly may refer to providing a disclosed compound or composition to a subject suffering from, or at risk of, the diseases or conditions to be treated or prevented. Herein, “subject,” “user,” “patient,” and “individual” are used interchangeably, and refer to any mammal, preferably a human. Such terms include one who has an indication for which a compound, composition, or method described herein may be efficacious, or who otherwise may benefit by the invention. In general, all of the compounds, compositions, and methods of the invention will be appreciated to work for all individuals, although individual variation is to be expected, and will be understood.

A “route of administration” may be or may include the path by which a compound or composition is taken into the body, or where it has its effect on the body. For example, enteral administration includes administration involving any part of the gastrointestinal tract. The examples may include those by mouth (orally), including but not limited to oral solid and oral liquid dosage forms, of which may be formulated as tablets, capsules, or drops; administered by gastric feeding tube, duodenal feeding tube, rectal administration, or gastrostomy. Parenteral administration includes administration from any means not involving the gastrointestinal tract, including but not limited to intravenous (into a vein), e.g., many drugs, total parenteral nutrition intra-arterial (into an artery), e.g., vasodilator drugs in the treatment of vasospasm and thrombolytic drugs for treatment of embolism; intraosseous infusion (into the bone marrow), intramuscular, intracerebral (into the brain parenchyma), intracerebroventricular (into cerebral ventricular system), intrathecal (an injection into the spinal canal), otic (through the ear), ocular (through the eye), vaginal, and subcutaneous (under the skin). In embodiments, parenteral administration may include sublingual and/or buccal administration. Among them, intraosseous infusion is, in effect, an indirect intravenous access because the bone marrow drains directly into the venous system. Intraosseous infusion may be occasionally used for drugs and fluids in emergency medicine and pediatrics when intravenous access is difficult. In embodiments, parenteral administration may include intraperitoneal injection or IP injection. Herein, “intraperitoneal injection” or “IP injection” may refer to the injection of a substance into the peritoneum (body cavity). IP injection is more often applied to animals than to humans. In general, IP injection may be preferred when large amounts of blood replacement fluids are needed, or when low blood pressure or other problems prevent the use of a suitable blood vessel for intravenous injection.

In embodiments, a disclosed entactogenic combination may be administered to a subject nasally or orally; for example, in oral solid and/or oral liquid dosage forms; via inhalation, using a nasal spray or oral inhaler; via nebulization, from a nebulizer; or buccally/sublingually.

In embodiments, a disclosed entactogenic combination may be administered to a subject via injection, wherein injection includes, but is not limited to, subcutaneous, intramuscular, intravenous, intrathecal, intraperitoneal, intraosseous, intracerebroventricular, or intra-arterial.

In embodiments, a disclosed entactogenic combination may be administered to a subject via transdermal application. In embodiments, a disclosed entactogenic combination may be administered to a subject via topical application. In embodiments, a disclosed entactogenic combination may be administered to a subject rectally. In embodiments, a disclosed entactogenic combination may be administered to a subject vaginally. In embodiments, a disclosed entactogenic combination may be administered to a subject ocularly. In embodiments, a disclosed entactogenic combination may be administered to a subject otically.

In embodiments, a disclosed entactogenic combination may be administered to the subject via enteral means, wherein, as mentioned, “enteral means” or “enteral administration means” includes, but is not limited to oral solid and oral liquid dosage forms, sublingual, rectal, and buccal administration means.

In embodiments, a disclosed entactogenic combination may be administered to a subject via parenteral means, wherein, as mentioned, “parenteral means” or “parenteral administration means” includes but is not limited to, intravenous, intra-arterial, intraperitoneal, intraosseous, intramuscular, intrathecal, intracerebroventricular, rectal, ocular, nasal, cutaneous, topical, otic, transdermal, and subcutaneous administration means.

In embodiments, a disclosed entactogenic combination may be administered to a subject via a combination of administration means. In embodiments, a disclosed entactogenic combination may be administered to a subject via one or more enteral administration means. In embodiments, a disclosed entactogenic combination may be administered to a subject via one or more parenteral administration means. In embodiments, the disclosed pharmaceutical composition may be administered to a subject via at least one enteral administration means, and at least one parenteral administration means. In some embodiments, an equivalent route of administration known to one of skill is utilized.

Compositions within the scope of the disclosure should be understood to be open-ended and may include additional active or inactive agents and ingredients. The type of formulation employed for the administration of the compounds employed in the disclosed methods generally may be dictated by the compound(s) employed, the type of pharmacokinetic profile desired from the route of administration and the compound(s), and the state of the patient. It will be readily appreciated that any of the above embodiments and classes of embodiments can be combined to form additional embodiments.

### K. Dosage

The invention provides methods for using therapeutically effective amounts of the disclosed pharmaceutical compositions in a mammal, and preferably a human. Such methods include those for treating a CNS disorder and for improving mental health and functioning, including in a healthy individual.

Administration of pharmaceutical compositions in an “effective amount,” a “therapeutically effective amount,” a “therapeutically effective dose,” or a “pharmacologically effective amount,” may refer to an amount of an active agent that is non-toxic and sufficient to provide the desired therapeutic effect with performance at a reasonable benefit/risk ratio attending any medical treatment. The effective amount will vary depending upon the subject and the disease condition being treated or health benefit sought, the weight and age of the subject, the severity of the disease condition or degree of health benefit sought, the manner of administration, and the like, all of which can readily be determined by one of skill.

In embodiments are disclosed entactogenic combinations comprising therapeutically effective amounts of a 5-HT₂ agent, 5-HT₇ agent, and/or a CB₁ agent, including pharmaceutically acceptable salts thereof, together with an I₁ agent, including pharmaceutically acceptable salts thereof, as disclosed herein. In embodiments, an “agent” may refer to any compound that binds to, blocks, activates, inhibits, or otherwise influences (e.g., via an allosteric reaction) activity at a given receptor.

In embodiments are disclosed pharmaceutical compositions comprising therapeutically effective amounts of any of a 5-HT₂ agent, 5-HT₇ agent, and/or a CB₁ agent, including pharmaceutically acceptable salts thereof, combined with an I₁ agent, including pharmaceutically acceptable salts thereof, together with a pharmaceutically acceptable carrier, diluent, or excipient.

In embodiments, the 5-HT₂ agent may be 1-methyl psilocin, 2C-B-FLY, 5-MeO-AMT, AL-37350A, CP 809101, DALT, DOB, DOET, DOHx, MEM, CPP, NBOH-2C-CN, TCB-2, or WAY 161503.

In embodiments, the 5-HT₇ agent may be 5-CT, 5-MeO-DALT, 5-MeO-DMT, 5-MeO-MiPT, AGH-192, AGH-107, AMT, aripiprazole, AS-19, E-55888, LSD, LP-211, or LP-44

In embodiments, the CB₁ receptor agent may be 2-AGE, A-834,735, ACEA, AZ-11713908, AZD-1940, CBN, CP-47497, CP55940, HU-210, JWH-007, JWH-051, JWH-146, JWH-176, JWH-200, JWH-398, Org 28611, rimonabant, THC, WIN 55,212-2, or XLR11.

In embodiments, the I₁ agent is agmatine, BDBM50091347, clonidine, guanfacine, mCPP, MDMA, memantine, moxonidine, naphazoline, oxymetazoline, rilmenidine, tetryzoline, tizanidine, or tolonidine.

In embodiments are therapeutic combinations comprising therapeutically effective amounts of a 5-HT₂ agent, including any of 1-methylpsilocin, 2C-B-FLY, 5-MeO-AMT, AL-37350A, CP 809101, DALT, DOB, DOET, DOHx, MEM, CPP, NBOH-2C-CN, TCB-2, and WAY 161503; and an I₁ agent, including any of agmatine, BDBM50091347, clonidine, guanfacine, mCPP, MDMA, memantine, moxonidine, naphazoline, oxymetazoline, rilmenidine, tetryzoline, tizanidine, or tolonidine.

In embodiments are therapeutic combinations comprising therapeutically effective amounts of a 5-HT₇ agent, including 5-CT, 5-MeO-DALT, 5-MeO-DMT, 5-MeO-MiPT, AGH-192, AGH-107, AMT, aripiprazole, AS-19, E-55888, LSD, LP-211, or LP-44; and an I₁ agent, including any of agmatine, BDBM50091347, clonidine, guanfacine, mCPP, MDMA, memantine, moxonidine, naphazoline, oxymetazoline, rilmenidine, tetryzoline, tizanidine, or tolonidine.

In embodiments are pharmaceutical compositions comprising therapeutically effective amounts of a 5-HT₂ agent, including any of 1-methylpsilocin, 2C-B-FLY, 5-MeO-AMT, AL-37350A, CP 809101, DALT, DOB, DOET, DOHx, MEM, CPP, NBOH-2C-CN, TCB-2, and WAY 161503; and an I₁ agent, including any of agmatine, BDBM50091347, clonidine, guanfacine, mCPP, MDMA, memantine, moxonidine, naphazoline, oxymetazoline, rilmenidine, tetryzoline, tizanidine, or tolonidine, together with a pharmaceutically acceptable carrier, diluent, or excipient.

In embodiments are pharmaceutical compositions comprising therapeutically effective amounts of a 5-HT₇ agent, including 5-CT, 5-MeO-DALT, 5-MeO-DMT, 5-MeO-MiPT, AGH-192, AGH-107, AMT, aripiprazole, AS-19, E-55888, LSD, LP-211, or LP-44; and an I₁ agent, including any of agmatine, BDBM50091347, clonidine, guanfacine, mCPP, MDMA, memantine, moxonidine, naphazoline, oxymetazoline, rilmenidine, tetryzoline, tizanidine, or tolonidine, together with a pharmaceutically acceptable carrier, diluent, or excipient.

In embodiments are therapeutic combinations comprising therapeutically effective amounts of a 5-HT₂ agent, including any of 1-methylpsilocin, 2C-B-FLY, 5-MeO-AMT, AL-37350A, CP 809101, DALT, DOB, DOET, DOHx, MEM, CPP, NBOH-2C-CN, TCB-2, and WAY 161503; an I₁ agent, including any of agmatine, BDBM50091347, clonidine, guanfacine, mCPP, MDMA, memantine, moxonidine, naphazoline, oxymetazoline, rilmenidine, tetryzoline, tizanidine, or tolonidine; and a CB₁ receptor agent, including any of 2-AGE, A-834,735, ACEA, AZ-11713908, AZD-1940, CBN, CP-47497, CP55940, HU-210, JWH-007, JWH-051, JWH-146, JWH-176, JWH-200, JWH-398, Org 28611, rimonabant, THC, WIN 55,212-2, and XLR11.

In embodiments are therapeutic combinations comprising therapeutically effective amounts of a 5-HT₇ agent, including 5-CT, 5-MeO-DALT, 5-MeO-DMT, 5-MeO-MiPT, AGH-192, AGH-107, AMT, aripiprazole, AS-19, E-55888, LSD, LP-211, or LP-44; an I₁ agent, including any of agmatine, BDBM50091347, clonidine, guanfacine, mCPP, MDMA, memantine, moxonidine, naphazoline, oxymetazoline, rilmenidine, tetryzoline, tizanidine, or tolonidine; and a CB₁ receptor agent, including any of 2-AGE, A-834,735, ACEA, AZ-11713908, AZD-1940, CBN, CP-47497, CP55940, HU-210, JWH-007, JWH-051, JWH-146, JWH-176, JWH-200, JWH-398, Org 28611, rimonabant, THC, WIN 55,212-2, and XLR11.

In embodiments are pharmaceutical compositions comprising therapeutically effective amounts of a 5-HT₂ agent, including any of 1-methylpsilocin, 2C-B-FLY, 5-MeO-AMT, AL-37350A, CP 809101, DALT, DOB, DOET, DOHx, MEM, CPP, NBOH-2C-CN, TCB-2, and WAY 161503; an I₁ agent, including any of agmatine, BDBM50091347, clonidine, guanfacine, mCPP, MDMA, memantine, moxonidine, naphazoline, oxymetazoline, rilmenidine, tetryzoline, tizanidine, or tolonidine; and a CB₁ receptor agent, including any of 2-AGE, A-834,735, ACEA, AZ-11713908, AZD-1940, CBN, CP-47497, CP55940, HU-210, JWH-007, JWH-051, JWH-146, JWH-176, JWH-200, JWH-398, Org 28611, rimonabant, THC, WIN 55,212-2, and XLR11, together with a pharmaceutically acceptable carrier, diluent, or excipient.

In embodiments are pharmaceutical compositions comprising therapeutically effective amounts of a 5-HT₇ agent, including 5-CT, 5-MeO-DALT, 5-MeO-DMT, 5-MeO-MiPT, AGH-192, AGH-107, AMT, aripiprazole, AS-19, E-55888, LSD, LP-211, or LP-44; an I₁ agent, including any of agmatine, BDBM50091347, clonidine, guanfacine, mCPP, MDMA, memantine, moxonidine, naphazoline, oxymetazoline, rilmenidine, tetryzoline, tizanidine, or tolonidine; and a CB₁ receptor agent, including any of 2-AGE, A-834,735, ACEA, AZ-11713908, AZD-1940, CBN, CP-47497, CP55940, HU-210, JWH-007, JWH-051, JWH-146, JWH-176, JWH-200, JWH-398, Org 28611, rimonabant, THC, WIN 55,212-2, and XLR11; together with a pharmaceutically acceptable carrier, diluent, or excipient.

In embodiments, the pharmaceutical compositions comprise other active or inactive ingredients.

Dosage amounts will be known by reference to the teachings herein and the general knowledge in the art, but certain exemplary dosage amounts, which may be useful in the practice of the invention, in some embodiments, are listed below for reference. It will be readily appreciated that where the disclosed entactogenic combination comprises a compound below formulated for an alternate form of administration, instead of the exemplary route of administration below, determination of the dose by total milligram, microgram, mg/kg, μg/kg, or other applicable amount will be made by the practice of ordinary skill, using the teachings herein and the general knowledge in the art as to such dose calculations or conversions. Unless specified below, the dose amounts listed are for the most common route of administration.

In embodiments, where the disclosed entactogenic combination comprises, for example, LSD, it may be present in an amount so that a single dose is (in a mg dosage amount), 25 μg or less (including a dose of 10 μg or less, 5 μg or less, 1 μg or less, and 0.5 μg or less), at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, at least 100 μg, at least 105 μg, at least 110 μg, at least 115 μg, at least 120 μg, at least 125 μg, at least 130 μg, at least 135 μg, at least 140 μg, at least 145 μg, at least 150 μg, at least 155 μg, at least 160 μg, at least 165 μg, at least 170 μg, at least 175 μg, at least 180 μg, at least 185 μg, at least 190 μg, at least 195 μg, at least 200 μg, at least 225 μg, at least 250 μg, as well as amounts within these ranges. In embodiments, where the disclosed entactogenic combination comprises, for example, LSD, it may be present in an amount so that a single dose is (in a microgram dosage amount) greater than 250 μg, including at least 275 μg, at least 300 μg, or at least 350 μg.

In embodiments, where the disclosed entactogenic combination comprises, for example, moxonidine, it may be present in an amount so that a single dose is (in a mg dosage amount), 0.2 mg or less, (including a dose of 0.19 or less, 0.18 mg or less, 0.17 mg or less, 0.16 mg or less, 0.15 mg or less, 0.14 mg or less, 0.13 mg or less, 0.12 mg or less, 0.11 mg or less, 0.1 mg or less, 0.09 mg or less, 0.08 mg or less, 0.07 mg or less, 0.06 mg or less, 0.05 mg or less, 0.04 mg or less, 0.03 mg or less, 0.02 mg or less, 0.01 mg or less, 0.005 mg or less, 0.001 mg or less) at least 0.21 mg, at least 0.22 mg, at least 0.23 mg, at least 0.24 mg, at least 0.25 mg, at least 0.26 mg, at least 0.27 mg, at least 0.28 mg, at least 0.29 mg, at least 0.3 mg, at least 0.31 mg, at least 0.32 mg, at least 0.33 mg, at least 0.34 mg, at least 0.35 mg, at least 0.36 mg, at least 0.37 mg, at least 0.38 mg, at least 0.39 mg, at least 0.4 mg, as well as amounts within these ranges. In embodiments, where the disclosed entactogenic combination comprises, for example, moxonidine, it may be present in an amount so that a single dose is (in a mg dosage amount) greater than 0.4 mg, including at least 0.5 mg, at least 0.75 mg, at least 0.9 mg, or at least 1 mg.

In embodiments, where the disclosed entactogenic combination comprises, for example, clonidine, it may be present in an amount so that a single dose is (in a mg dosage amount), 0.5 mg or less, (including a dose of 0.4 or less, 0.3 mg or less, 0.2 mg or less, 0.1 mg or less, 0.09 mg or less, 0.08 mg or less, 0.07 mg or less, 0.06 mg or less, 0.05 mg or less, 0.04 mg or less, 0.03 mg or less, 0.02 mg or less, 0.01 mg or less, 0.005 mg or less, 0.001 mg or less) at least 0.5 mg, at least 0.6 mg, at least 0.7 mg, at least 0.8 mg, at least 0.9 mg, at least 1 mg, at least 1.1 mg, at least 1.2 mg, at least 1.3 mg, at least 1.4 mg, at least 1.5 mg, at least 1.6 mg, at least 1.7 mg, at least 1.8 mg, at least 1.9 mg, at least 2 mg, at least 2.1 mg, at least 2.2 mg, at least 2.3 mg, at least 2.4 mg, at least 2.5 mg, at least 2.6 mg, at least 2.7 mg, at least 2.8 mg, at least 2.9 mg, at least 3 mg, at least 3.1 mg, at least 3.2 mg, at least 3.3 mg, at least 3.4 mg, at least 3.5 mg, at least 3.6 mg, at least 3.7 mg, at least 3.8 mg, at least 3.9 mg, at least 4 mg, and values in between. In embodiments, where the disclosed entactogenic combination comprises, for example, clonidine, it may be present in an amount so that a single dose is (in a mg dosage amount) greater than 4 mg, including at least 5 mg, or at least 7 mg. In embodiments, where the disclosed entactogenic combination comprises, for example, clonidine, it may be present in an amount so that a single dose is about 0.2 mg.

In embodiments, where the disclosed entactogenic combination comprises, for example, DOB, guanfacine, rilmenidine, or tolonidine, it may be present in an amount so that a single dose is (in a mg dosage amount), 0.1 mg or less (including a dose of 0.07 mg or less, 0.05 mg or less, 0.01 mg or less, and 0.005 mg or less), at least 0.1 mg, at least 0.2 mg, at least 0.3 mg, at least 0.4 mg, at least 0.5 mg, at least 0.6 mg, at least 0.7 mg, at least 0.8 mg, at least 0.9 mg, at least 1 mg, at least 1.1 mg, at least 1.2 mg, at least 1.3 mg, at least 1.4 mg, at least 1.5 mg, at least 1.6 mg, at least 1.7 mg, at least 1.8 mg, at least 1.9 mg, at least 2 mg, at least 2.1 mg, at least 2.2 mg, at least 2.3 mg, at least 2.4 mg, at least 2.5 mg, at least 2.6 mg, at least 2.7 mg, at least 2.8 mg, at least 2.9 mg, at least 3 mg, at least 3.1 mg, at least 3.2 mg, at least 3.3 mg, at least 3.4 mg, at least 3.5 mg, at least 3.6 mg, at least 3.7 mg, at least 3.8 mg, at least 3.9 mg, at least 4 mg, at least 4.1 mg, at least 4.2 mg, at least 4.3 mg, at least 4.4 mg, at least 4.5 mg, at least 4.6 mg, at least 4.7 mg, at least 4.8 mg, at least 4.9 mg, at least 5 mg, at least 5.1 mg, at least 5.2 mg, at least 5.3 mg, at least 5.4 mg, at least 5.5 mg, at least 5.6 mg, at least 5.7 mg, at least 5.8 mg, at least 5.9 mg, at least 6 mg, at least 6.1 mg, at least 6.2 mg, at least 6.3 mg, at least 6.4 mg, at least 6.5 mg, at least 6.6 mg, at least 6.7 mg, at least 6.8 mg, at least 6.9 mg, at least 7 mg, at least 7.1 mg, at least 7.2 mg, at least 7.3 mg, at least 7.4 mg, at least 7.5 mg, at least 7.6 mg, at least 7.7 mg, at least 7.8 mg, at least 7.9 mg, at least 8 mg, as well as amounts within these ranges. In embodiments, where the disclosed entactogenic combination comprises, for example, DOB, guanfacine, rilmenidine, or tolonidine, it may be present in an amount so that a single dose is (in a mg dosage amount) greater than 8 mg, including at least 10 mg, at least 12 mg, or at least 15 mg.

In embodiments, where the disclosed entactogenic combination comprises, for example, DOET or BDBM50091347, it may be present in an amount so that a single dose is (in a mg dosage amount), 1 mg or less (including a dose of 0.5 mg or less, 0.25 mg or less, 0.1 mg or less, 0.05 mg or less, 0.005 mg or less, 0.001 mg or less, and 0.0005 mg or less), at least 1.1 mg, at least 1.2 mg, at least 1.3 mg, at least 1.4 mg, at least 1.5 mg, at least 1.6 mg, at least 1.7 mg, at least 1.8 mg, at least 1.9 mg, at least 2 mg, at least 2.1 mg, at least 2.2 mg, at least 2.3 mg, at least 2.4 mg, at least 2.5 mg, at least 2.6 mg, at least 2.7 mg, at least 2.8 mg, at least 2.9 mg, at least 3 mg, at least 3.1 mg, at least 3.2 mg, at least 3.3 mg, at least 3.4 mg, at least 3.5 mg, at least 3.6 mg, at least 3.7 mg, at least 3.8 mg, at least 3.9 mg, at least 4 mg, at least 4.1 mg, at least 4.2 mg, at least 4.3 mg, at least 4.4 mg, at least 4.5 mg, at least 4.6 mg, at least 4.7 mg, at least 4.8 mg, at least 4.9 mg, at least 5 mg, at least 5.1 mg, at least 5.2 mg, at least 5.3 mg, at least 5.4 mg, at least 5.5 mg, at least 5.6 mg, at least 5.7 mg, at least 5.8 mg, at least 5.9 mg, at least 6 mg, at least 6.1 mg, at least 6.2 mg, at least 6.3 mg, at least 6.4 mg, at least 6.5 mg, at least 6.6 mg, at least 6.7 mg, at least 6.8 mg, at least 6.9 mg, at least 7 mg, at least 7.1 mg, at least 7.2 mg, at least 7.3 mg, at least 7.4 mg, at least 7.5 mg, at least 7.6 mg, at least 7.7 mg, at least 7.8 mg, at least 7.9 mg, at least 8 mg, at least 8.1 mg, at least 8.2 mg, at least 8.3 mg, at least 8.4 mg, at least 8.5 mg, at least 8.6 mg, at least 8.7 mg, at least 8.8 mg, at least 8.9 mg, at least 9 mg, at least 9.5 mg, at least 10 mg, as well as amounts within these ranges. In embodiments, where the disclosed entactogenic combination comprises, for example, DOET or BDBM50091347, it may be present in an amount so that a single dose is (in a mg dosage amount) greater than 10 mg, including at least 15 mg, or at least 20 mg.

In embodiments, where the disclosed entactogenic combination comprises, for example, 5-MEO-MiPT, it may be present in an amount so that a single dose is (in a milligram (i.e., a “mg”) dosage amount), 2 mg or less (including a dose of 1.5 mg or less, 1.25 mg or less, 1 mg or less, 0.5 mg or less, 0.05 mg or less, 0.01 mg or less, and 0.005 mg or less), at least 2 mg, at least 2.5 mg, at least 3 mg, at least 3.5 mg, at least 4 mg, at least 4.5 mg, at least 5 mg, at least 5.5 mg, at least 6 mg, at least 6.5 mg, at least 7 mg, at least 7.5 mg, at least 8 mg, at least 8.5 mg, at least 9 mg, at least 9.5 mg, at least 10 mg, at least 10.5 mg, at least 11 mg, at least 11.5 mg, at least 12 mg, at least 12.5 mg, at least 13 mg, at least 13.5 mg, at least 14 mg, at least 14.5 mg, at least 15 mg, at least 15.5 mg, at least 16 mg, at least 16.5 mg, at least 17 mg, at least 17.5 mg, at least 18 mg, at least 18.5 mg, at least 19 mg, at least 19.5 mg, at least 20 mg, at least 20.5 mg, at least 21 mg, at least 21.5 mg, at least 22 mg, at least 22.5 mg, at least 23 mg, at least 23.5 mg, at least 24 mg, at least 24.5 mg, at least 25 mg, as well as amounts within these ranges. In embodiments, where the disclosed entactogenic combination comprises, for example, 5-MEO-MiPT, it may be present in an amount so that a single dose is (in a mg dosage amount) greater than 25 mg, including at least 33 mg, at least 37 mg, or at least 40 mg.

In embodiments, where the disclosed entactogenic combination comprises, for example, DOHx, 5-MeO-DMT, NBOH-2C-CN, CP 809101, TCB-2, 1-methylpsilocin, CPP, WAY 161503, 5-MeO-AMT, AL-37350A, 5-CT, AGH-192, AGH-107, AS-19, LP-211, LP-44, A-834,735, AZ-11713908, AZD-1940, CP-47497, CP55940, HU-210, JWH-007, JWH-051, JWH-146, JWH-176, JWH-200, JWH-398, Org 28611, 2C-B-FLY, or WIN 55,212-2, it may be present in an amount so that a single dose is (in a mg dosage amount), 1 mg or less (including a dose of 0.5 mg or less, 0.25 mg or less, 0.1 mg or less, 0.05 mg or less, 0.005 mg or less, 0.001 mg or less, and 0.0005 mg or less), at least 1 mg, at least 1.5 mg, at least 2 mg, at least 2.5 mg, at least 3 mg, at least 3.5 mg, at least 4 mg, at least 4.5 mg, at least 5 mg, at least 5.5 mg, at least 6 mg, at least 6.5 mg, at least 7 mg, at least 7.5 mg, at least 8 mg, at least 8.5 mg, at least 9 mg, at least 9.5 mg, at least 10 mg, at least 10.5 mg, at least 11 mg, at least 11.5 mg, at least 12 mg, at least 12.5 mg, at least 13 mg, at least 13.5 mg, at least 14 mg, at least 14.5 mg, at least 15 mg, at least 15.5 mg, at least 16 mg, at least 16.5 mg, at least 17 mg, at least 17.5 mg, at least 18 mg, at least 18.5 mg, at least 19 mg, at least 19.5 mg, at least 20 mg, at least 20.5 mg, at least 21 mg, at least 21.5 mg, at least 22 mg, at least 22.5 mg, at least 23 mg, at least 23.5 mg, at least 24 mg, at least 24.5 mg, at least 25 mg, at least 25.5 mg, at least 26 mg, at least 26.5 mg, at least 27 mg, at least 27.5 mg, at least 28 mg, at least 28.5 mg, at least 29 mg, at least 29.5 mg, at least 30 mg, as well as amounts within these ranges. In embodiments, where the disclosed entactogenic combination comprises, for example, OHx, 5-MeO-DMT, NBOH-2C-CN, CP 809101, TCB-2, 1-methylpsilocin, CPP, WAY 161503, 5-MeO-AMT, AL-37350A, 5-CT, AGH-192, AGH-107, AS-19, LP-211, LP-44, A-834,735, AZ-11713908, AZD-1940, CP-47497, CP55940, HU-210, JWH-007, JWH-051, JWH-146, JWH-176, JWH-200, JWH-398, Org 28611, 2C-B-FLY, or WIN 55,212-2, it may be present in an amount so that a single dose is (in a mg dosage amount) greater than 35 mg, including at least 40 mg, or at least 50 mg.

In embodiments, where the disclosed entactogenic combination comprises, for example, MEM, 5-MeO-DALT, memantine, or tizanidine, it may be present in an amount so that a single dose is (in a mg dosage amount), 5 mg or less (including a dose of 4.5 mg or less, 4 mg or less, 3.5 mg or less, 3 mg or less, 2.5 mg or less, 2 mg or less, 1.5 mg or less, 1 mg or less, 0.5 mg or less), at least 5 mg, at least 6 mg, at least 7 mg, at least 8 mg, at least 9 mg, at least 10 mg, at least 11 mg, at least 12 mg, at least 13 mg, at least 14 mg, at least 15 mg, at least 16 mg, at least 17 mg, at least 18 mg, at least 19 mg, at least 20 mg, at least 21 mg, at least 22 mg, at least 23 mg, at least 24 mg, at least 25 mg, at least 26 mg, at least 27 mg, at least 28 mg, at least 29 mg, at least 30 mg, at least 31 mg, at least 32 mg, at least 33 mg, at least 34 mg, at least 35 mg, at least 36 mg, at least 37 mg, at least 38 mg, at least 39 mg, at least 40 mg, at least 41 mg, at least 42 mg, at least 43 mg, at least 44 mg, at least 45 mg, at least 46 mg, at least 47 mg, at least 48 mg, at least 49 mg, at least 50 mg, at least 51 mg, at least 52 mg, at least 53 mg, at least 54 mg, at least 55 mg, at least 56 mg, at least 57 mg, at least 58 mg, at least 59 mg, at least 60 mg, at least 61 mg, at least 62 mg, at least 63 mg, at least 64 mg, at least 65 mg, at least 66 mg, at least 67 mg, at least 68 mg, at least 69 mg, at least 70 mg, at least 71 mg, at least 72 mg, at least 73 mg, at least 74 mg, at least 75 mg, as well as amounts within these ranges. In embodiments, where the disclosed entactogenic combination comprises, for example, MEM, 5-MeO-DALT, memantine, or tizanidine, it may be present in an amount so that a single dose is (in a mg dosage amount) greater than 75 mg, including at least 85 mg, at least 90 mg, or at least 100 mg.

In embodiments, where the disclosed entactogenic combination comprises, for example, mCPP or MDMA, it may be present in an amount so that a single dose is (in a mg dosage amount), 15 mg or less (including a dose of 10 mg or less, 5 mg or less, 1 mg or less, and 0.5 mg or less), at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 105 mg, at least 110 mg, at least 115 mg, at least 120 mg, at least 125 mg, at least 130 mg, at least 135 mg, at least 140 mg, at least 145 mg, at least 150 mg, at least 155 mg, at least 160 mg, at least 165 mg, at least 170 mg, at least 175 mg, as well as amounts within these ranges. In embodiments, where the disclosed entactogenic combination comprises, for example, mCPP or MDMA, it may be present in an amount so that a single dose is (in a mg dosage amount) greater than 175 mg, including at least 200 mg, at least 225 mg, or at least 250 mg.

In embodiments, where the disclosed entactogenic combination comprises, for example, DALT, AMT, aripiprazole, E-55888, 2-AGE, ACEA, CBN, rimonabant, THC, or XLR-11, it may be present in an amount so that a single dose is (in a mg dosage amount), 25 mg or less (including a dose of 10 mg or less, 5 mg or less, 1 mg or less, and 0.5 mg or less), at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 105 mg, at least 110 mg, at least 115 mg, at least 120 mg, at least 125 mg, at least 130 mg, at least 135 mg, at least 140 mg, at least 145 mg, at least 150 mg, at least 155 mg, at least 160 mg, at least 165 mg, at least 170 mg, at least 175 mg, at least 180 mg, at least 185 mg, at least 190 mg, at least 195 mg, at least 200 mg, at least 225 mg, at least 250 mg, as well as amounts within these ranges. In embodiments, where the disclosed entactogenic combination comprises, for example, DALT, AMT, aripiprazole, E-55888, 2-AGE, ACEA, CBN, rimonabant, THC, or XLR-11, it may be present in an amount so that a single dose is (in a mg dosage amount) greater than 250 mg, including at least 275 mg, at least 300 mg, or at least 350 mg.

In embodiments, where the disclosed entactogenic combination comprises, for example, agmatine, it may be present in an amount so that a single dose is (in a mg dosage amount), 300 mg or less (including a dose of 200 mg or less, 150 mg or less, 100 mg or less, and 50 mg or less), at least 300 mg, at least 310 mg, at least 320 mg, at least 330 mg, at least 340 mg, at least 350 mg, at least 360 mg, at least 370 mg, at least 380 mg, at least 390 mg, at least 400 mg, at least 410 mg, at least 420 mg, at least 430 mg, at least 440 mg, at least 450 mg, at least 460 mg, at least 470 mg, at least 480 mg, at least 490 mg, at least 500 mg, at least 510 mg, at least 520 mg, at least 530 mg, at least 540 mg, at least 550 mg, at least 560 mg, at least 570 mg, at least 580 mg, at least 590 mg, at least 600 mg, at least 610 mg, at least 620 mg, at least 630 mg, at least 640 mg, at least 650 mg, at least 660 mg, at least 670 mg, at least 680 mg, at least 690 mg, at least 700 mg, as well as amounts within these ranges. In embodiments, where the disclosed entactogenic combination comprises, for example, agmatine, it may be present in an amount so that a single dose is (in a mg dosage amount) greater than 700 mg, including at least 750 mg, at least 800 mg, or at least 850 mg.

In embodiments, where the disclosed entactogenic combination comprises, for example, naphazoline, a single dosage comprises 1 to 2 drops, of 0.1% solution in the conjunctival sacs every 3 to 4 hours as needed. As it relates to the invention, it is understood that a single drop delivers between 62 μL to 80 μL. Thus, a single dose is between 62 μL to 160 μL, with a maximum daily amount of between 372 μL to 1,280 μL (Hakim et al., 2016). Thus, in embodiments, where the disclosed entactogenic combination comprises, for example, naphazoline, a single dosage comprises 100 μL or less, including 90 μL or less, 70 μL or less, 50 μL or less, 25 μL or less, 10 μL or less) at least 100 μL, at least 110 μL, at least 120 μL, at least 130 μL, at least 140 μL, at least 150 μL, at least 160 μL, at least 170 μL, at least 180 μL, at least 190 μL, at least 200 μL, at least 210 μL, at least 220 μL, at least 230 μL, at least 240 μL, at least 250 μL, at least 260 μL, at least 270 μL, at least 280 μL, at least 290 μL, at least 300 μL, at least 310 μL, at least 320 μL, at least 330 μL, at least 340 μL, at least 350 μL, as well as amounts within these ranges. In embodiments, where the disclosed entactogenic combination comprises, for example, naphazoline, it may be present in an amount so that a single dose is greater than 350 μL, including at least 375 μL, at least 400 μL, or at least 450 μL. In embodiments, where the disclosed entactogenic combination comprises, for example, naphazoline formulated for an alternate form of administration, such as oral administration, the determination of dosage by total mg amount or mg/kg amount will be made by the practice of ordinary skill. In such embodiments, the dose is (in a mg dosage amount), 0.3 mg or less, (including a dose of 0.2 mg or less, 0.1 mg or less, 0.09 mg or less, 0.08 mg or less, 0.07 mg or less, 0.06 mg or less, 0.05 mg or less, 0.04 mg or less, 0.03 mg or less, 0.02 mg or less, 0.01 mg or less, 0.005 mg or less, 0.001 mg or less) at least 0.3 mg, at least 0.4 mg, at least 0.5 mg, at least 0.6 mg, at least 0.7 mg, at least 0.8 mg, at least 0.9 mg, at least 1 mg, at least 1.1 mg, at least 1.2 mg, at least 1.3 mg, at least 1.4 mg, at least 1.5 mg, at least 1.6 mg, at least 1.7 mg, at least 1.8 mg, at least 1.9 mg, at least 2 mg, at least 2.1 mg, at least 2.2 mg, at least 2.3 mg, at least 2.4 mg, at least 2.5 mg, at least 2.6 mg, at least 2.7 mg, at least 2.8 mg, at least 2.9 mg, at least 3 mg, at least 3.1 mg, at least 3.2 mg, at least 3.3 mg, at least 3.4 mg, at least 3.5 mg, and values in between.

In embodiments, where the disclosed entactogenic combination comprises, for example, oxymetazoline, a single dosage comprises 2 to 3 drops, or 2 to 3 sprays of 0.05% solution in each nostril every ten to twelve hours, with the maximum dose being a single dose, as mentioned above, administered to a subject twice in a period of twenty four hours. As it relates to the invention, it is understood that a single drop, or a single spray of the bottle from an upright position delivers between 62 μL to 80 μL. Thus, a single dose is between 124 μL to 240 μL (Hakim et al., 2016). Thus, in embodiments, where the disclosed entactogenic combination comprises, for example, oxymetazoline, a single dosage comprises 100 μL or less, including 90 μL or less, 70 μL or less, 50 μL or less, 25 μL or less, 10 μL or less) at least 100 μL, at least 110 μL, at least 120 μL, at least 130 μL, at least 140 μL, at least 150 μL, at least 160 μL, at least 170 μL, at least 180 μL, at least 190 μL, at least 200 μL, at least 210 μL, at least 220 μL, at least 230 μL, at least 240 μL, at least 250 μL, at least 260 μL, at least 270 μL, at least 280 μL, at least 290 μL, at least 300 μL, at least 310 μL, at least 320 μL, at least 330 μL, at least 340 μL, at least 350 μL, as well as amounts within these ranges. In embodiments, where the disclosed entactogenic combination comprises, for example, oxymetazoline, it may be present in an amount so that a single dose is greater than 350 μL, including at least 375 μL, at least 400 μL, or at least 450 μL. In embodiments, where the disclosed entactogenic combination comprises, for example, oxymetazoline formulated for an alternate form of administration, such as oral administration, the determination of dosage by total mg amount or mg/kg amount will be made by the practice of ordinary skill. In such embodiments, the dose is (in a mg dosage amount), 0.5 mg or less, (including a dose of 0.4 or less, 0.3 mg or less, 0.2 mg or less, 0.1 mg or less, 0.09 mg or less, 0.08 mg or less, 0.07 mg or less, 0.06 mg or less, 0.05 mg or less, 0.04 mg or less, 0.03 mg or less, 0.02 mg or less, 0.01 mg or less, 0.005 mg or less, 0.001 mg or less) at least 0.5 mg, at least 0.6 mg, at least 0.7 mg, at least 0.8 mg, at least 0.9 mg, at least 1 mg, at least 1.1 mg, at least 1.2 mg, at least 1.3 mg, at least 1.4 mg, at least 1.5 mg, at least 1.6 mg, at least 1.7 mg, at least 1.8 mg, at least 1.9 mg, at least 2 mg, at least 2.1 mg, at least 2.2 mg, at least 2.3 mg, at least 2.4 mg, at least 2.5 mg, at least 2.6 mg, at least 2.7 mg, at least 2.8 mg, at least 2.9 mg, at least 3 mg, at least 3.1 mg, at least 3.2 mg, at least 3.3 mg, at least 3.4 mg, at least 3.5 mg, at least 3.6 mg, at least 3.7 mg, at least 3.8 mg, at least 3.9 mg, at least 4 mg, and values in between.

In embodiments, where the disclosed entactogenic combination comprises, for example, tetryzoline, a single dosage comprises 1 to 2 drops, of 0.05% solution in each eye up to four times daily. As it relates to the invention, it is understood that a single drop delivers between 62 μL to 80 μL. Thus, a single dose is between 62 μL to 160 μL, with a maximum daily amount of between 248 μL to 640 μL (Hakim et al., 2016). Thus, in embodiments, where the disclosed entactogenic combination comprises, for example, tetryzoline, a single dosage comprises 100 μL or less, including 90 μL or less, 70 μL or less, 50 μL or less, 25 μL or less, 10 μL or less) at least 100 μL, at least 110 μL, at least 120 μL, at least 130 μL, at least 140 μL, at least 150 μL, at least 160 μL, at least 170 μL, at least 180 μL, at least 190 μL, at least 200 μL, at least 210 μL, at least 220 μL, at least 230 μL, at least 240 μL, at least 250 μL, at least 260 μL, at least 270 μL, at least 280 μL, at least 290 μL, at least 300 μL, at least 310 μL, at least 320 μL, at least 330 μL, at least 340 μL, at least 350 μL, as well as amounts within these ranges. In embodiments, where the disclosed entactogenic combination comprises, for example, tetryzoline, it may be present in an amount so that a single dose is greater than 350 μL, including at least 375 μL, at least 400 μL, at least 450 μL, at least 500 μL, at least 550 μL, or at least 600 μL. In embodiments, where the disclosed entactogenic combination comprises tetryzoline formulated for an alternate form of administration, such as oral administration, the determination of dosage by total mg amount or mg/kg amount will be made by the practice of ordinary skill. In such embodiments, the dose is (in a mg dosage amount), 0.5 mg or less, (including a dose of 0.4 or less, 0.3 mg or less, 0.2 mg or less, 0.1 mg or less, 0.09 mg or less, 0.08 mg or less, 0.07 mg or less, 0.06 mg or less, 0.05 mg or less, 0.04 mg or less, 0.03 mg or less, 0.02 mg or less, 0.01 mg or less, 0.005 mg or less, 0.001 mg or less) at least 0.5 mg, at least 0.6 mg, at least 0.7 mg, at least 0.8 mg, at least 0.9 mg, at least 1 mg, at least 1.1 mg, at least 1.2 mg, at least 1.3 mg, at least 1.4 mg, at least 1.5 mg, at least 1.6 mg, at least 1.7 mg, at least 1.8 mg, at least 1.9 mg, at least 2 mg, at least 2.1 mg, at least 2.2 mg, at least 2.3 mg, at least 2.4 mg, at least 2.5 mg, at least 2.6 mg, at least 2.7 mg, at least 2.8 mg, at least 2.9 mg, at least 3 mg, at least 3.1 mg, at least 3.2 mg, at least 3.3 mg, at least 3.4 mg, at least 3.5 mg, at least 3.6 mg, at least 3.7 mg, at least 3.8 mg, at least 3.9 mg, at least 4 mg, and values in between.

In embodiments, the dosage of the compounds described above administered to, or taken by a patient in need thereof is a “microdose” (whether or not so named), wherein the dose is not perceptible by the patient to which it is administered. In embodiments, a microdose may be 0.001 to 0.25 of a macro-dose, such as but not limited to 0.003, 0.005, 0.007, 0.009, 0.01, 0.03, 0.05, 0.07, 0.09, 0.1, 0.2, and 0.25 of a macrodose (wherein the list is inclusive, and modified by the term “about”). Necessarily, then, a person of skill will be able to determine a sufficient dose for a patient based on the specific entactogenic combination administered to the same. Meaning, if the appropriate dose for a patient in need thereof is 100 mg, a microdose may be between 0.1 mg to 20 mg. Hence, determining a microdose appropriate for a patient for each of the aforementioned compounds is within the skill of, and should be readily apparent to, one of skill.

In embodiments, a patient will be administered a microdose of the disclosed entactogenic combination on a regular or “chronic” basis, wherein the patient is administered the combination or composition daily, several times per day (at least one, at least two, at least three, at least four, or greater than four times per day); on a set, repeating schedule, wherein the patient is administered a microdose of the combination or composition every other day, every three days, every four days, every five days, every six days, every seven days, or more than every seven days; or, in embodiments, a varying schedule comprises of a plurality of days “on” (wherein the microdose of the combination or composition is administered), and a plurality of days “off” (wherein no administration occurs), such as one day on two days off, two days on three days off, three days on four days off, or other such schedules as would be apparent to those of skill (cf., e.g., the Stamets or Fadimen microdosing protocols).

In embodiments, a therapeutically effective amount of a pharmaceutical composition comprising a therapeutically effective amount of an I₁ agent, and a therapeutically effective amount of any of a CB₁ agent, a 5-HT₂ agent, and/or a 5-HT₇ agent are administered to a subject.

In embodiments, administration is part of a psychedelic-assisted therapy session.

In embodiments, the pharmaceutical composition is administered as part of a combination therapy, wherein the therapeutically effective amount of an I₁ agent, and the therapeutically effective amount of any of a CB₁ agent, a 5-HT₂ agent, or a 5-HT₇ agent are administered to the patient via separate means wherein the means are any of: oral, including oral solid and oral liquid forms; buccally, sublingually, through injection, including intravenous, intra-arterial, intraperitoneal, intraosseous, intramuscular, intrathecal, intracerebroventricular, and subcutaneous injections; administered rectally, vaginally, ocularly, nasally, inhaled, as a vapor such as through a vaporizer device or a vape pen, or through combusted smoke, through a pipe, a cigarette, a cigar, a blunt, or a dab; cutaneously, otically, transdermally, through a patch, hydrogel, or liquid bandage formulation; topically, through a salve, aerosol, emulsion, spray, ointment, gel, paste, lotion, liniment, oil or cream, or a combination thereof, as would be immediately appreciated by one of skill. Additionally, one of skill may further choose an administration method known to be equivalent to those discussed herein.

In embodiments, a patient is administered a therapeutically effective amount of a pharmaceutical composition comprising a therapeutically effective amount of an I₁ agent, and a therapeutically effective amount of any of a CB₁ agent, a 5-HT₂ agent, or a 5-HT₇ agent, wherein the therapeutically effective amount of the I₁ agent is agmatine.

In embodiments, a patient is administered a therapeutically effective amount of a pharmaceutical composition comprising a therapeutically effective amount of an I₁ agent, and a therapeutically effective amount of any of a CB₁ agent, a 5-HT₂ agent, or a 5-HT₇ agent, wherein the therapeutically effective amount of the I₁ agent is BDBM50091347.

In embodiments, a patient is administered a therapeutically effective amount of a pharmaceutical composition comprising a therapeutically effective amount of an I₁ agent, and a therapeutically effective amount of any of a CB₁ agent, a 5-HT₂ agent, or a 5-HT₇ agent, wherein the therapeutically effective amount of the I₁ agent is clonidine.

In embodiments, a patient is administered a therapeutically effective amount of a pharmaceutical composition comprising a therapeutically effective amount of an I₁ agent, and a therapeutically effective amount of any of a CB₁ agent, a 5-HT₂ agent, or a 5-HT₇ agent, wherein the therapeutically effective amount of the I₁ agent is guanfacine.

In embodiments, a patient is administered a therapeutically effective amount of a pharmaceutical composition comprising a therapeutically effective amount of an I₁ agent, and a therapeutically effective amount of any of a CB₁ agent, a 5-HT₂ agent, or a 5-HT₇ agent, wherein the therapeutically effective amount of the I₁ agent is mCPP.

In embodiments, a patient is administered a therapeutically effective amount of a pharmaceutical composition comprising a therapeutically effective amount of an I₁ agent, and a therapeutically effective amount of any of a CB₁ agent, a 5-HT₂ agent, or a 5-HT₇ agent, wherein the therapeutically effective amount of the I₁ agent is MDMA.

In embodiments, a patient is administered a therapeutically effective amount of a pharmaceutical composition comprising a therapeutically effective amount of an I₁ agent, and a therapeutically effective amount of any of a CB₁ agent, a 5-HT₂ agent, or a 5-HT₇ agent, wherein the therapeutically effective amount of the I₁ agent is memantine.

In embodiments, a patient is administered a therapeutically effective amount of a pharmaceutical composition comprising a therapeutically effective amount of an I₁ agent, and a therapeutically effective amount of any of a CB₁ agent, a 5-HT₂ agent, or a 5-HT₇ agent, wherein the therapeutically effective amount of the I₁ agent is moxonidine.

In embodiments, a patient is administered a therapeutically effective amount of a pharmaceutical composition comprising a therapeutically effective amount of an I₁ agent, and a therapeutically effective amount of any of a CB₁ agent, a 5-HT₂ agent, or a 5-HT₇ agent, wherein the therapeutically effective amount of the I₁ agent is naphazoline.

In embodiments, a patient is administered a therapeutically effective amount of a pharmaceutical composition comprising a therapeutically effective amount of an I₁ agent, and a therapeutically effective amount of any of a CB₁ agent, a 5-HT₂ agent, or a 5-HT₇ agent, wherein the therapeutically effective amount of the I₁ agent is oxymetazoline.

In embodiments, a patient is administered a therapeutically effective amount of a pharmaceutical composition comprising a therapeutically effective amount of an I₁ agent, and a therapeutically effective amount of any of a CB₁ agent, a 5-HT₂ agent, or a 5-HT₇ agent, wherein the therapeutically effective amount of the I₁ agent is rilmenidine.

In embodiments, a patient is administered a therapeutically effective amount of a pharmaceutical composition comprising a therapeutically effective amount of an I₁ agent, and a therapeutically effective amount of any of a CB₁ agent, a 5-HT₂ agent, or a 5-HT₇ agent, wherein the therapeutically effective amount of the I₁ agent is tetryzoline.

In embodiments, a patient is administered a therapeutically effective amount of a pharmaceutical composition comprising a therapeutically effective amount of an I₁ agent, and a therapeutically effective amount of any of a CB₁ agent, a 5-HT₂ agent, or a 5-HT₇ agent, wherein the therapeutically effective amount of the I₁ agent is tolonidine.

In embodiments, a patient is administered a therapeutically effective amount of a pharmaceutical composition comprising a therapeutically effective amount of an I₁ agent, and a therapeutically effective amount of any of a CB₁ agent, a 5-HT₂ agent, or a 5-HT₇ agent, wherein the therapeutically effective amount of the I₁ agent is tizanidine.

In embodiments, a patient is administered a therapeutically effective amount of a pharmaceutical composition comprising a therapeutically effective amount of an I₁ agent, and a therapeutically effective amount of a CB₁, agent, wherein the therapeutically effective amount of the CB₁ agent is 2-AGE.

In embodiments, a patient is administered a therapeutically effective amount of a pharmaceutical composition comprising a therapeutically effective amount of an I₁ agent, and a therapeutically effective amount of a CB₁, agent, wherein the therapeutically effective amount of the CB₁ agent is A-834,735.

In embodiments, a patient is administered a therapeutically effective amount of a pharmaceutical composition comprising a therapeutically effective amount of an I₁ agent, and a therapeutically effective amount of a CB₁, agent, wherein the therapeutically effective amount of the CB₁ agent is ACEA.

In embodiments, a patient is administered a therapeutically effective amount of a pharmaceutical composition comprising a therapeutically effective amount of an I₁ agent, and a therapeutically effective amount of a CB₁, agent, wherein the therapeutically effective amount of the CB₁ agent is AZ-11713908.

In embodiments, a patient is administered a therapeutically effective amount of a pharmaceutical composition comprising a therapeutically effective amount of an I₁ agent, and a therapeutically effective amount of a CB₁, agent, wherein the therapeutically effective amount of the CB₁ agent is AZD-1940.

In embodiments, a patient is administered a therapeutically effective amount of a pharmaceutical composition comprising a therapeutically effective amount of an I₁ agent, and a therapeutically effective amount of a CB₁, agent, wherein the therapeutically effective amount of the CB₁ agent is CBN.

In embodiments, a patient is administered a therapeutically effective amount of a pharmaceutical composition comprising a therapeutically effective amount of an I₁ agent, and a therapeutically effective amount of a CB₁, agent, wherein the therapeutically effective amount of the CB₁ agent is CP-47497.

In embodiments, a patient is administered a therapeutically effective amount of a pharmaceutical composition comprising a therapeutically effective amount of an I₁ agent, and a therapeutically effective amount of a CB₁, agent, wherein the therapeutically effective amount of the CB₁ agent is CP55940.

In embodiments, a patient is administered a therapeutically effective amount of a pharmaceutical composition comprising a therapeutically effective amount of an I₁ agent, and a therapeutically effective amount of a CB₁, agent, wherein the therapeutically effective amount of the CB₁ agent is HU-210.

In embodiments, a patient is administered a therapeutically effective amount of a pharmaceutical composition comprising a therapeutically effective amount of an I₁ agent, and a therapeutically effective amount of a CB₁, agent, wherein the therapeutically effective amount of the CB₁ agent is JWH-007.

In embodiments, a patient is administered a therapeutically effective amount of a pharmaceutical composition comprising a therapeutically effective amount of an I₁ agent, and a therapeutically effective amount of a CB₁, agent, wherein the therapeutically effective amount of the CB₁ agent is JWH-051.

In embodiments, a patient is administered a therapeutically effective amount of a pharmaceutical composition comprising a therapeutically effective amount of an I₁ agent, and a therapeutically effective amount of a CB₁, agent, wherein the therapeutically effective amount of the CB₁ agent is JWH-146.

In embodiments, a patient is administered a therapeutically effective amount of a pharmaceutical composition comprising a therapeutically effective amount of an I₁ agent, and a therapeutically effective amount of a CB₁, agent, wherein the therapeutically effective amount of the CB₁ agent is JWH-176.

In embodiments, a patient is administered a therapeutically effective amount of a pharmaceutical composition comprising a therapeutically effective amount of an I₁ agent, and a therapeutically effective amount of a CB₁, agent, wherein the therapeutically effective amount of the CB₁ agent is JWH-200.

In embodiments, a patient is administered a therapeutically effective amount of a pharmaceutical composition comprising a therapeutically effective amount of an I₁ agent, and a therapeutically effective amount of a CB₁, agent, wherein the therapeutically effective amount of the CB₁ agent is JWH-398.

In embodiments, a patient is administered a therapeutically effective amount of a pharmaceutical composition comprising a therapeutically effective amount of an I₁ agent, and a therapeutically effective amount of a CB₁, agent, wherein the therapeutically effective amount of the CB₁ agent is Org 28611.

In embodiments, a patient is administered a therapeutically effective amount of a pharmaceutical composition comprising a therapeutically effective amount of an I₁ agent, and a therapeutically effective amount of a CB₁, agent, wherein the therapeutically effective amount of the CB₁ agent is rimonabant.

In embodiments, a patient is administered a therapeutically effective amount of a pharmaceutical composition comprising a therapeutically effective amount of an I₁ agent, and a therapeutically effective amount of a CB₁, agent, wherein the therapeutically effective amount of the CB₁ agent is THC.

In embodiments, a patient is administered a therapeutically effective amount of a pharmaceutical composition comprising a therapeutically effective amount of an I₁ agent, and a therapeutically effective amount of a CB₁, agent, wherein the therapeutically effective amount of the CB₁ agent is WIN 55,212-2.

In embodiments, a patient is administered a therapeutically effective amount of a pharmaceutical composition comprising a therapeutically effective amount of an I₁ agent, and a therapeutically effective amount of a CB₁, agent, wherein the therapeutically effective amount of the CB₁ agent is XLR11.

In embodiments, a patient is administered a therapeutically effective amount of a pharmaceutical composition comprising a therapeutically effective amount of an I₁ agent, and a therapeutically effective amount of a 5-HT₂, agent, wherein the therapeutically effective amount of the 5-HT₂ agent is 1-methylpsilocin.

In embodiments, a patient is administered a therapeutically effective amount of a pharmaceutical composition comprising a therapeutically effective amount of an I₁ agent, and a therapeutically effective amount of a 5-HT₂, agent, wherein the therapeutically effective amount of the 5-HT₂ agent is 5-MeO-AMT.

In embodiments, a patient is administered a therapeutically effective amount of a pharmaceutical composition comprising a therapeutically effective amount of an I₁ agent, and a therapeutically effective amount of a 5-HT₂, agent, wherein the therapeutically effective amount of the 5-HT₂ agent is 2C-B-FLY.

In embodiments, a patient is administered a therapeutically effective amount of a pharmaceutical composition comprising a therapeutically effective amount of an I₁ agent, and a therapeutically effective amount of a 5-HT₂, agent, wherein the therapeutically effective amount of the 5-HT₂ agent is AL-37350A.

In embodiments, a patient is administered a therapeutically effective amount of a pharmaceutical composition comprising a therapeutically effective amount of an I₁ agent, and a therapeutically effective amount of a 5-HT₂, agent, wherein the therapeutically effective amount of the 5-HT₂ agent is DALT.

In embodiments, a patient is administered a therapeutically effective amount of a pharmaceutical composition comprising a therapeutically effective amount of an I₁ agent, and a therapeutically effective amount of a 5-HT₂, agent, wherein the therapeutically effective amount of the 5-HT₂ agent is DOB.

In embodiments, a patient is administered a therapeutically effective amount of a pharmaceutical composition comprising a therapeutically effective amount of an I₁ agent, and a therapeutically effective amount of a 5-HT₂, agent, wherein the therapeutically effective amount of the 5-HT₂ agent is MEM.

In embodiments, a patient is administered a therapeutically effective amount of a pharmaceutical composition comprising a therapeutically effective amount of an I₁ agent, and a therapeutically effective amount of a 5-HT₂, agent, wherein the therapeutically effective amount of the 5-HT₂ agent is DOET.

In embodiments, a patient is administered a therapeutically effective amount of a pharmaceutical composition comprising a therapeutically effective amount of an I₁ agent, and a therapeutically effective amount of a 5-HT₂, agent, wherein the therapeutically effective amount of the 5-HT₂ agent is DOHx.

In embodiments, a patient is administered a therapeutically effective amount of a pharmaceutical composition comprising a therapeutically effective amount of an I₁ agent, and a therapeutically effective amount of a 5-HT₂, agent, wherein the therapeutically effective amount of the 5-HT₂ agent is NBOH-2C-CN.

In embodiments, a patient is administered a therapeutically effective amount of a pharmaceutical composition comprising a therapeutically effective amount of an I₁ agent, and a therapeutically effective amount of a 5-HT₂, agent, wherein the therapeutically effective amount of the 5-HT₂ agent is CP 809101.

In embodiments, a patient is administered a therapeutically effective amount of a pharmaceutical composition comprising a therapeutically effective amount of an I₁ agent, and a therapeutically effective amount of a 5-HT₂, agent, wherein the therapeutically effective amount of the 5-HT₂ agent is TCB-2.

In embodiments, a patient is administered a therapeutically effective amount of a pharmaceutical composition comprising a therapeutically effective amount of an I₁ agent, and a therapeutically effective amount of a 5-HT₂, agent, wherein the therapeutically effective amount of the 5-HT₂ agent is CPP.

In embodiments, a patient is administered a therapeutically effective amount of a pharmaceutical composition comprising a therapeutically effective amount of an I₁ agent, and a therapeutically effective amount of a 5-HT₂, agent, wherein the therapeutically effective amount of the 5-HT₂ agent is WAY 161503.

In embodiments, a patient is administered a therapeutically effective amount of a pharmaceutical composition comprising a therapeutically effective amount of an I₁ agent, and a therapeutically effective amount of a 5-HT₇, agent, wherein the therapeutically effective amount of the 5-HT₇ agent is 5-MeO-DMT.

In embodiments, a patient is administered a therapeutically effective amount of a pharmaceutical composition comprising a therapeutically effective amount of an I₁ agent, and a therapeutically effective amount of a 5-HT₇, agent, wherein the therapeutically effective amount of the 5-HT₇ agent is 5-MeO-DALT.

In embodiments, a patient is administered a therapeutically effective amount of a pharmaceutical composition comprising a therapeutically effective amount of an I₁ agent, and a therapeutically effective amount of a 5-HT₇ agent, wherein the therapeutically effective amount of the 5-HT₇ agent is 5-MeO-MiPT.

In embodiments, a patient is administered a therapeutically effective amount of a pharmaceutical composition comprising a therapeutically effective amount of an I₁ agent, and a therapeutically effective amount of a 5-HT₇ agent, wherein the therapeutically effective amount of the 5-HT₇ agent is 5-CT.

In embodiments, a patient is administered a therapeutically effective amount of a pharmaceutical composition comprising a therapeutically effective amount of an I₁ agent, and a therapeutically effective amount of a 5-HT₇ agent, wherein the therapeutically effective amount of the 5-HT₇ agent is aripiprazole.

In embodiments, a patient is administered a therapeutically effective amount of a pharmaceutical composition comprising a therapeutically effective amount of an I₁ agent, and a therapeutically effective amount of a 5-HT₇ agent, wherein the therapeutically effective amount of the 5-HT₇ agent is AS-19.

In embodiments, a patient is administered a therapeutically effective amount of a pharmaceutical composition comprising a therapeutically effective amount of an I₁ agent, and a therapeutically effective amount of a 5-HT₇ agent, wherein the therapeutically effective amount of the 5-HT₇ agent is E-55888.

In embodiments, a patient is administered a therapeutically effective amount of a pharmaceutical composition comprising a therapeutically effective amount of an I₁ agent, and a therapeutically effective amount of a 5-HT₇ agent, wherein the therapeutically effective amount of the 5-HT₇ agent is LP-44.

In embodiments, a patient is administered a therapeutically effective amount of a pharmaceutical composition comprising a therapeutically effective amount of an I₁ agent, and a therapeutically effective amount of a 5-HT₇ agent, wherein the therapeutically effective amount of the 5-HT₇ agent is LP-211.

In embodiments, a patient is administered a therapeutically effective amount of a pharmaceutical composition comprising a therapeutically effective amount of an I₁ agent, and a therapeutically effective amount of a 5-HT₇ agent, wherein the therapeutically effective amount of the 5-HT₇ agent is AMT.

In embodiments, a patient is administered a therapeutically effective amount of a pharmaceutical composition comprising a therapeutically effective amount of an I₁ agent, and a therapeutically effective amount of a 5-HT₇ agent, wherein the therapeutically effective amount of the 5-HT₇ agent is LSD.

In embodiments, a patient is administered a therapeutically effective amount of a pharmaceutical composition comprising a therapeutically effective amount of an I₁ agent, and a therapeutically effective amount of a 5-HT₇ agent, wherein the therapeutically effective amount of the 5-HT₇ agent is AGH-107.

In embodiments, a patient is administered a therapeutically effective amount of a pharmaceutical composition comprising a therapeutically effective amount of an I₁ agent, and a therapeutically effective amount of a 5-HT₇ agent, wherein the therapeutically effective amount of the 5-HT₇ agent is AGH-192.

It will be readily appreciated that dosages may vary depending upon whether the treatment is therapeutic or prophylactic, the onset, progression, severity, frequency, duration, probability of or susceptibility of the symptom to which treatment is directed, clinical endpoint desired, previous, simultaneous or subsequent treatments, general health, age, gender, and race of the subject, bioavailability, potential adverse systemic, regional or local side effects, the presence of other disorders or diseases in the subject, and other factors that will be appreciated by the skilled artisan (e.g., medical or familial history).

Dose amount, frequency or duration may be increased or reduced, as indicated by the clinical outcome desired, status of the pathology or symptom, any adverse side effects of the treatment or therapy, or concomitant medications. The skilled artisan with the teaching of this disclosure in hand will appreciate the factors that may influence the dosage, frequency, and timing required to provide an amount sufficient or effective for providing a therapeutic effect or benefit, and to do so depending on the type of therapeutic effect desired, as well as to avoid or minimize adverse effects.

It will be understood that, in embodiments, the dose actually administered will be determined by a physician, in light of the relevant circumstances, including the disorder to be treated, the chosen route of administration, the actual composition or formulation administered, the age, weight, and response of the individual patient, the severity of the patient's symptoms, and taking into consideration such “objective measurements” discussed herein, and therefore any dosage ranges disclosed herein are not intended to limit the scope of the disclosure. In some instances, dosage levels below the lower limit of a disclosed range may be more than adequate, while in other cases doses above a range may be employed without causing any harmful side effects, provided for instance that such larger doses also may be divided into several smaller doses for administration, either taken together or separately.

In these embodiments, the disclosed pharmaceutical compositions will be administered and dosed in accordance with good medical practice, taking into account the method and scheduling of administration, prior and concomitant medications and medical supplements, the clinical condition of the individual patient and the severity of the underlying disease, the patient's age, sex, body weight, and other such factors relevant to medical practitioners, and knowledge of the particular compound(s) used, including such “objective measurements” discussed herein. Starting and maintenance dosage levels thus may differ from patient to patient, for individual patients across time, and for different pharmaceutical compositions and formulations, but shall be able to be determined with ordinary skill.

In embodiments, a patient will be evaluated prior to being administered (or, in embodiments, being told to self-administer) the disclosed pharmaceutical compositions. In embodiments, the evaluation may include completing questionnaires, obtaining objective health measurements (herein “objective measurements”) from the patient, including but not limited to weight, body temperature, heart rate (HR), respiratory rate, blood oxygenation, blood pressure (BP) and its variables, including, but not limited to: systolic (SBP), diastolic (DBP), mean arterial (MAP), and pulse (PP); continuous non-invasive beat-by-beat blood pressure (CNIBP); measurements from an electrocardiogram (ECG), including RR interval or its variability, QT interval or its variability, heart rate variability (HRV) (or measured by devices other than an ECG); hemodynamic response (HR), and levels of glucose, cortisol, serotonin, dopamine, cholesterol; electroencephalography (EEG) measures such as quantitative EEG (qEEG); electrocochleogram (ECochG), electromyography (EMG), electrooculography (EOG), magnetoencephalography (MEG); electrocorticography (ECoG); magnetic resonance imaging (MRI); functional MRI (fMRI); computed tomography (CT); positron emission tomography (PET); nuclear magnetic resonance (NMR) spectroscopy or magnetic resonance spectroscopy (MSR); single-photon emission computed tomography (SPECT); near infrared spectroscopy (NIRS); event-related optical signal (EROS); computed axial tomography; diffuse optical imaging (DOI); cranial ultrasound; or functional ultrasound imaging (fUS) (together, “EEG measures”); brain derived neurotrophic factor (BDNF); genetic markers including relating to CYP enzymes or drug metabolism; genetic variation in mGluR5 or FKBP5; and assessing the patient for “biomarkers,” such as but not limited to those that may indicate poor metabolism of a psychedelic drug, such as but not limited to the CYP2D6 and CYP2B6 genes; those that may impact the response to serotonin, such as but not limited to the HTR2A gene; genes that may pose a mental health risk, such as but not limited to the C4A, NRG1, and DISC1 genes; and physiological fluctuations that may indicate a patient will not perceive the full psychedelic experience, including but not limited to having a higher diversity of executive network nodes (i.e., less efficient network segregation), and having a lesser rostral anterior cingulate (rACC) thickness; and numerous more, as will be appreciated by those of skill.

It should be appreciated that, in other embodiments—for example when the compositions of the invention are taken without the direct intervention or guidance of a medical professional—appropriate dosages to achieve a therapeutic effect, including the upper and lower bounds of any dose ranges, can be determined by an individual by reference to available public information and knowledge, and reference to subjective considerations regarding desired outcomes and effects.

Determination of appropriate dosing shall include not only the determination of single dose amounts, but also the determination of the number and timing of doses, e.g., administration of a particular dosage amount once per day, twice per day, or more than twice per day, and the time(s) of day or time(s) during treatment preferable for their administration.

In embodiments, especially where a formulation is prepared in single unit dosage form, such as a capsule, tablet, or lozenge, suggested dosages may be known by reference to the format of the preparation itself. In other embodiments, where a formulation is prepared in multiple dosage form, for instance liquid suspensions and topical preparations, suggested dosage amounts may be known by reference to the means of administration or by reference to the packaging and labeling, package insert(s), marketing materials, training materials, or other information and knowledge available to those of skill or the public.

### L. Pharmaceutical Kits

In embodiments, especially where a combination is prepared in single unit dosage form, suggested dosage amounts shall be known by reference to the format of the preparation itself. In other embodiments, suggested dosage amounts may be known by reference to the means of administration or by reference to the packaging and labeling, package insert(s), marketing materials, training materials, or other information and knowledge available to those of skill or the public. Another aspect of this disclosure therefore provides pharmaceutical kits, containers, and articles of manufacture comprising a therapeutic combination as provided herein. In embodiments, the pharmaceutical kits, containers, and articles of manufacture comprise suggested administration guidelines or prescribing information therefor.

In embodiments, individual unit dosage forms can be included in multi-dose kits or containers. In embodiments, disclosed combinations and compositions can be packaged in single or multiple unit dosage forms for uniformity of dosage and ease of administration. Accordingly, another aspect of this disclosure provides pharmaceutical kits containing a disclosed combination or composition, suggested administration guidelines or prescribing information therefore, and a suitable container. Individual unit dosage forms can be included in multi-dose kits or containers. In embodiments, any of the containers provided herein protect the contents therein from light, for example, UV irradiation.

In an exemplary pharmaceutical kit, capsules, tablets, caplets, or other unit dosage forms are packaged in blister packs. “Blister pack” may refer to any of several types of pre-formed container, especially plastic packaging, that contains separate receptacles (e.g., cavities or pockets) for single unit doses, where such separate receptacles are individually sealed and can be opened individually. Blister packs thus include such pharmaceutical blister packs known to those of ordinary skill, including Aclar® Rx160, Rx20e, SupRx, and UltRx 2000, 3000, 4000, and 6000 (Honeywell). Within the definition of multi-dose containers, and also often referred to as blister packs, are blister trays, blister cards, strip packs, push-through packs, and the like. Preferably, information pertaining to dosing and proper administration (if needed) will be printed onto a multi-dose kit directly (e.g., on a blister pack or other interior packaging holding the compositions or formulations of the invention); however, kits of the invention can further contain package inserts and other printed instructions (e.g., on exterior packaging) for administering the compositions of the invention and for their appropriate therapeutic use.

In embodiments, disclosed kits comprise packaging materials for use in packaging pharmaceutical products such as known in the art, and described, e.g., in U.S. Pat. Nos. 5,323,907, 5,052,558 and 5,033,252, each of which is incorporated herein in its entirety. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment, including for example vape carts. The choice of package depends on the agents. In general, the packaging is non-reactive with the compositions contained therein.

In embodiments, the agents provided herein, such as an I₁ agent and one or more of a 5-HT₂ agent, 5-HT₇ agent, and CB₁ agent can be packaged in different containers. In embodiments, the agents provided herein are packaged in the same container. In embodiments, the agents packed in the same container are separately sealed to prevent interaction between the agents. Any container or other article of manufacture is contemplated, so long as the agents are separated from the other components prior to administration. For suitable embodiments see, e.g., containers described in U.S. Pat. Nos. 3,539,794 and 5,171,081. In embodiments, a plurality of containers are provided, each separately containing an I₁ agent and one or more of a 5-HT₂ agent, 5-HT₇ agent, and CB₁ agent. In embodiments, the container(s) provided herein can be packed together as a kit, such as a pharmaceutical kit.

In embodiments, a patient will have the option of using online software such as a website, or downloadable software such as a mobile application, to assist with compliance or to provide data relating to treatment. Such software can be used to, e.g., keep track of last dose taken and total doses taken, provide reminders and alerts for upcoming doses, provide feedback to discourage taking doses outside of set schedules, and allow for recording of specific subjective effects, or provide means for unstructured journaling. Such data collection can assist with individual patient compliance, can be used to improve or tailor individual patient care plans, and can be anonymized, aggregated, and analyzed (including by AI or natural language processing means) to allow research into the effects of various methods of treatment.

### M. Methods of Treatment

In some embodiments, a disclosed entactogenic combination may be utilized as part of “psychedelic-assisted therapy.” Psychedelic-assisted therapy, broadly, includes a range of related approaches that involve at least one session where the patient ingests an agent referred to as a “psychedelic” and is monitored, supported, and/or otherwise engaged by one or more trained mental health professionals while under the effects of the psychedelic (see, e.g., Schenberg 2018). It will be readily appreciated that when the term “psychedelic-assisted therapy” is used, or the term “psychedelic” used in relation thereto, a drug compound used in the therapy need not be a “psychedelic” as the class is understood as that of “classic” psychedelics, but may include drugs like MDMA (an entactogen) and ketamine (a dissociative anesthetic). Accordingly, when “psychedelic-assisted therapy” or the term “psychedelic” in relation thereto is used herein, it will be understood that the terms may include a disclosed entactogenic combination, which nonetheless may be more properly termed an “entactogen” or “entactogenic combination” used in “entactogen-assisted therapy” or “entactogen-assisted psychotherapy” (and elsewhere is so termed, although all terms may be used, solely for purposes of shorthand, and not of any specific equivalency, such as pharmacological equivalency).

Methods of MDMA-assisted psychotherapy will be known to those in the field and are disclosed, for example, in Grof (2008) LSD Psychotherapy (Ben Lomond, CA: Multidisciplinary Association for Psychedelic Studies); Passie (2012) Healing with Entactogens: Therapist and Patient Perspectives on MDMA-Assisted Group Psychotherapy (Ben Lomond, CA: Multidisciplinary Association for Psychedelic Studies); Johnson, Richards, & Griffiths (2008. Human hallucinogen research: guidelines for safety. Journal of psychopharmacology, 22(6), 603-620); Sessa & Fischer (2015, Underground MDMA-, LSD-and 2-CB-assisted individual and group psychotherapy in Zurich: Outcomes, implications and commentary. Drug Science, Policy and Law, 2, 2050324515578080); Schmid, Gasser, Oehen, & Liechti (2020. Acute subjective effects in LSD- and MDMA-assisted psychotherapy. Journal of psychopharmacology, 0269881120959604); Greer & Tolbert (1998. A method of conducting therapeutic sessions with MDMA. Journal of psychoactive drugs, 30(4), 371-379); Mithoefer et al. A Manual for MDMA-Assisted Therapy in the Treatment of PTSD (2017); Mithoefer (2013. MDMA-assisted psychotherapy: How different is it from other psychotherapy. Manifesting minds: A review of psychedelics in science, medicine, sex, and spirituality, 125)); see also U.S. Pat. App. No. 2020/0360311A1, generally discussing drug-assisted psychotherapy practices, which can be applied with MDMA.

Generally, PAT includes one or more psychedelic dosing session(s), one or more preparation sessions before the one or more psychedelic dosing session(s), and one or more integration sessions after the psychedelic dosing session(s). It will be readily appreciated that the number and relative timing and order of the sessions will be chosen based on the therapeutic goal(s), the protocol(s), clinical manual(s), or REMS aspect(s) followed, the psychedelic(s) used, the characteristics of the patient(s) and the disorder(s) to be treated (or improvements in mental health sought), and such other characteristics as will be appreciated by those of skill.

In embodiments, accordingly, it will be appreciated that a disclosed entactogenic combination is provided alone, and without psychotherapy or psychological support. In embodiments, a disclosed entactogenic combination is provided solely with monitoring, for example before and during the duration of drug effects, during the duration of drug effects, during the duration of peak drug effects, during the duration of drug effects and for a specified time thereafter, for a specified time after administration of the drug, and the like. Such “monitoring,” whereby a patient is observed to ensure, for example, adherence to any clinical protocol(s) and safety, may occur for example by a human in physical proximity to the patient, or capable of becoming in physical proximity to the patient; in other embodiments, a patient may be monitored entirely remotely, such as by one or more human monitors, or solely by a computer means or AI. Systems and methods of monitoring those undergoing PAP are found in US2020/0147038A1 and US2021/0183519A1.

“Psychotherapy” refers generally to any method for treating mental health problems by interacting or talking with a psychiatrist, psychologist or other mental health provider, such as the therapeutic approach used by MAPS to treat PTSD patients using MDMA (e.g., Mithoefer 2017), psychodynamic therapies, psychoanalytic therapies, psychosocial or behavioral therapies, including any of (or adapted from any of) cognitive behavioral therapy (e.g., as described in Arch. Gen. Psychiatry 1999; 56:493-502), interpersonal therapy (e.g., as described in Psychol Addict Behav 2009; 23(1): 168-174), contingency management based therapy (e.g., as described in Psychol Addict Behav 2009; 23(1): 168-174; in J. Consul. Clin. Psychol. 2005; 73(2): 354-59; or in Case Reports in Psychiatry, Vol. 2012, Article ID 731638), motivational interviewing based therapy (e.g., as described in J. Consul. Clin. Psychol. 2001; 69(5): 858-62), or meditation based therapy, such as transcendental meditation based therapy (e.g., as described in J. Consul. Clin. Psychol. 2000; 68(3): 515-52), eye movement desensitization and reprocessing (EMDR) therapy, and others, as will be readily appreciated.

“Psychological support” refers generally to any method to ensure a patient's physical and psychological safety and minimize the number and severity of potential adverse events during a drug-administration session, such as by providing a comfortable environment or safe “container” (“setting”), but without necessarily involving some form of direct psychotherapeutic intervention; such methods may for example include “self directed enquiry,” where a therapist helps a patient direct attention toward internal experiences as they emerge in the present moment, which may involve noticing foreground and background thoughts, emotions, physical sensations, images and memories, with the aim to bring experiences into awareness and potentially consider them from different perspectives; and “experiential processing” which may refer to the sustaining of attention on an experience in the present moment so it can be processed in a meaningful way, i.e., by staying with thoughts, feelings, sensations or emotions until they have passed or evolved (moving “in and through”), and where the resulting awareness of and re-evaluation of important biological, social, and psychological variables can potentiate new insights and facilitate change (see, e.g., Carhart-Harris 2016; Carhart-Harris 2017; Tai 2021). It will be readily appreciated that “psychotherapy” and “psychological support” need not, in embodiments, be mutually exclusive, and for example “psychotherapy” may include aspects of “psychological support” (just as both may include aspects of “monitoring”).

Terms like “therapist,” “psychoanalyst,” “psychiatrist,” “clinician,” “psychologist,” “researcher,” “facilitator,” “guide,” “shaman,” and the like may be used in reference to users of or participants in embodiments described herein, these terms are largely interchangeable as well as non-limiting, and should be understood to apply in the plural as well as the singular (e.g., a team of therapists, clinicians, or researchers or even the scientific community at large as well as a sole therapist, clinician, or researcher).

While terms like “subject,” “patient,” “candidate,” “client,” “individual,” and the like may be used in reference to the person being studied or treated in disclosed embodiments, these terms are largely interchangeable as well as non-limiting, and will be understood to apply in the plural as well as the singular (e.g., a group or “cohort” of subjects, patients, or candidates as in group therapy, as well as a sole subject, patient, or candidate). It will be readily appreciated that therapy can therefore include in embodiments individual therapy, couples therapy, group therapy, and combinations thereof, as known to those of skill.

In embodiments disclosed herein, a patient is administered a drug during a drug-assisted session, according to the disclosed methods and/or using the devices or systems of the invention, without (or with optional) preparation and/or integration sessions. Accordingly, the methods, devices, and systems of the invention, as will be described below, may be practiced and used during a drug-assisted session, without regard to whether a patient will also participate in one or more preparation and/or integration session (although it will be appreciated that clinical outcomes may differ, and may be improved, where the patient participates in preparation and/or integration, and that clinical outcomes may also differ, and may be improved, where the patient also undergoes one or more psychotherapeutic modalities during treatment, and is not simply administered a drug without further support).

In embodiments, an “entactogenic combination” may be used interchangeably with “therapeutic combination” or “pharmaceutical composition,” and also may include both, which shall be determined by context. In embodiments herein, administration of a disclosed entactogenic combination will provide a therapeutic effect. A therapeutic effect may be the treatment of a disease or condition. Where “an entactogenic combination” provides an effect, or is used in a disclosed method, it will be understood that the combination also may provide an equivalent effect, or be used in an equivalent method, when the agents in the combination (each separately, or one or more together, including all together) are formulated with a carrier, diluent, or excipient, or otherwise be prepared as a pharmaceutical composition.

In embodiments, a disclosed entactogenic combination is used in a method of treating a diseases or condition, such as post-traumatic stress disorder (PTSD), depression, including major depressive disorder (MDD) and treatment-resistant depression (TRD); borderline personality disorder, bipolar disorder and bipolar depression, premenstrual dysphoric disorder, substance abuse, anxiety disorder, addiction, and obsessive compulsive disorder (OCD). As discussed above, such treatment may be completed as part of a psychedelic-assisted therapy session, or may be privately completed by the patient, including by administration of the entactogenic combination alone, or together with one or more therapeutically beneficial activities performed by the patient, such as meditation, yoga, breathwork, journaling, and such other activities discussed below and known in the art.

In some instances, certain personalized approaches (i.e., “personalized” or “precision” medicine) are utilized, based on individual characteristics, including in some embodiments drug metabolism (e.g., CYP2B6, CYP1A2, CYP2C19, CYP2D6, or CYP3A4) or individual genetic variation. The term “genetic variation” may refer to a change in a gene sequence relative to a reference sequence (e.g., a commonly-found and/or wild-type sequence). Genetic variation may be recombination events or mutations such as substitution/deletion/insertion events like point and splice site mutations. In embodiments, the genetic variation is a genetic variation in one or more cytochrome P450 (CYP or CYP450) enzymes that affects drug metabolism, including metabolism of a disclosed composition, and including CYP1A2, CYP2C9, CYP2D6, CYP2C19, CYP3A4 and CYP3A5. Other examples of CYP enzymes include CYP1A1, CYP1B1, CYP2A6, CYP2A13, CYP2B6, CYP2C8, CYP2C9, CYP2C18, CYP2E1, CYP2G1, CYP2J2, CYP2R1, CYP2S1, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51. In embodiments, a patient will be included or excluded from therapy based on a genetic variation. In embodiments, a patient therapy will be modified based on a genetic variation.

In embodiments, a disclosed composition is taken together with a compound that is metabolized by the same CYP enzyme(s) as the disclosed composition, so as to permit a lower dose to be taken, increase the effective bioavailability of one or both, or otherwise affect drug metabolism or pharmacokinetics. In embodiments, the dose of a disclosed composition is adjusted when administered to a subject known to be a “poor metabolizer” of the active agent in composition (e.g., having a genetic variation in CYP2D6, known to be the major metabolizer of the methylenedioxy moiety). In embodiments, a genetic variation is an exclusion criteria for the administration of a disclosed compound.

In embodiments, the patient will be assessed for “biomarkers” that may indicate a patient will have a suboptimal response to psychedelic treatment, including those that may indicate poor metabolism of a psychedelic drug, such as but not limited to the CYP2D6 and CYP2B6 genes; those that may impact the response to serotonin, such as but not limited to the HTR2A gene; genes that may pose a mental health risk, such as but not limited to the C4A, NRG1, and DISC1 genes; and physiological fluctuations that may indicate a patient will not perceive the full psychedelic experience, including but not limited to having a higher diversity of executive network nodes (i.e., less efficient network segregation), and having a lesser rostral anterior cingulate (rACC) thickness; and numerous more, as readily appreciated by those of skill.

In one embodiment, the genetic variation is a genetic variation in metabotropic glutamate receptor type 5 (mGluR5), which has been implicated in mood and anxiety symptoms in humans. In another embodiment, the genetic variation is one or more single nucleotide polymorphisms (SNPs) in the FKBP5 gene that are associated with elevated levels of FKBP51 protein relative to persons lacking such SNPs. The FKBP5 gene has been implicated in responses to stress and trauma, and such SNPs are correlated with susceptibility to certain depression, PTSD, and anxiety disorders. In one embodiment, the genetic variation is a genetic variation such as a SNP in a membrane transporter, such as SERT, DAT, NET, or VMAT.

In one embodiment, the mammal being treated has altered epigenetic regulation of a gene the expression of which is associated with a mental health condition or susceptibility to a mental health treatment, such as the SIGMAR1 gene for the non-opioid sigma-1 receptor. Herein, “an effective amount” or “a pharmacologically effective amount” may refer to an amount of an active agent that is non-toxic and sufficient to provide the desired therapeutic effect with performance at a reasonable benefit/risk ratio attending any medical treatment. The effective amount will vary depending upon the subject and the disease condition being treated or health benefit sought, the weight and age of the subject, the severity of the disease condition or degree of health benefit sought, the manner of administration, and the like, all of which can readily be determined by one of skill.

Measures of therapeutic effect includes any outcome measure, endpoint, effect measure, or measure of effect within clinical or medical practice or research which is used to assess the effect, both positive and negative, of an intervention or treatment, whether patient-reported (e.g., questionnaires, patient testimonies, etc.), based on other patient data (e.g., patient monitoring), gathered through laboratory tests such as blood work, urine samples, etc., through medical examination by a doctor or other medical professional, or by digital tools or means, e.g., electronic tools such as online tools, smartphones, wireless devices, biosensors, or health apps. In embodiments, measures of therapeutic effect will include an assessment. “Assessment” may refer to any means or method used with a patient, whether before, during, after, or unrelated in time to a specific treatment protocol, to measure, estimate, or evaluate a nature, ability, symptom, disorder, or other characteristic of the patient, whether qualitatively or quantitatively, and whether performed by the therapist or other clinician (e.g., an interview), by the patient his or herself (e.g., a self-reported questionnaire), by a third-party or by a computer, including a medical device (e.g., as such as defined by the FDA or other regulatory body) or other device (e.g., a medical sensor or biosensor, a watch or fitness tracker, or a “wearable”), and whether graded by a human decision-maker or an artificial intelligence, machine learning, or computer algorithm, and whether or not regulated as software as a medical device (SaMD). Non-limiting examples of assessments include those in Table 1 below (and others are described herein, or will be readily known to those of ordinary skill).

TABLE 1
Exemplary Patient Assessments

| | |
|---|---|
| 1 | Mini International Neuropsychiatric Interview 5 |
| | (MINI 5) (Sheehan et al. 1998) to screen for |
| | comorbid psychiatric disorders. |
| 2 | Columbia Suicide Severity Rating Scale |
| | (C-SSRS) (Mundt, JC et al. 2013), screens for acute and |
| | recent suicide and self-harm thoughts and |
| | behaviors, taking approximately 5 minutes to complete. |
| 3 | Patient Health Questionnaire (PHQ-9) (Kroenke |
| | et al. 2001). A brief self-administered screening |
| | questionnaire for depressive symptoms. |
| 4 | Generalized Anxiety Disorder 7 (GAD-7) (Spitzer |
| | et al. 2006) is a self-reported questionnaire for |
| | screening and severity measuring of |
| | generalized anxiety disorder. |
| 5 | Pittsburgh Sleep Quality Index (PSQI) (Buysse |
| | 1989) assesses the level of sleep disturbance. |
| 6 | Interpersonal reactivity Index (IRI) (Davis 1980) |
| | comprises 28 items answered on a 5 point scale. |
| | This scale measures different aspects of empathy |
| | and provides different subscales relating to these. |
| 7 | Short Form (36) Health Survey (SF-36) is a |
| | standard patient-reported measure of quality of life. |
| 8 | Self-Compassion Scale (SCS) (Neff 2003) |
| | Comprises 26 items answered on a 5 point scale. This |
| | scale measures core aspects of self-compassion |
| | including components of mindfulness. |
| 9 | Trauma History Questionnaire (THQ) (Green |
| | 1996) is a self-report measure that examines |
| | experiences with potentially traumatic events |
| | using a yes/no format. For each event endorsed, |
| | respondents are asked to provide the frequency |
| | of the event and their age at the time of the event. |

An assessment may be computer-assisted, and other computer-assisted assessments may be performed besides the assessments above. The term “computer-assisted” in “computer-assisted assessment” means an assessment comprising the use of electronic tools such as online tools, smartphones, wireless devices, or health apps (in some such examples, also known as “digital phenotyping”). Computer-assisted assessment will include the use of an electronic psychiatric notes system, where relevant clinical information will be recorded for the duration of the therapy by a therapist interacting face-to-face with a patient, and will also include the use of computer systems where the therapist and patient interact virtually (either synchronously or asynchronously), as well as where a patient only interacts with a computer (“computer” broadly meaning any electronic tool suitable for such purposes, including desktop, laptop, and notebook computers; tablets, smartphones, and other mobile devices; watches, fitness trackers, and personal electronic devices; and the like).

In some embodiments, the invention provides methods of treating and/or preventing a condition in a mammal, the method comprising administering to the mammal a therapeutically effective and/or prophylactically effective amount of a formulation with one or more active agents. Herein, “treating” or “treatment” covers any treatment of a disorder in a mammal, and preferably in a human, and includes causing a desired biological or pharmacological effect as above, as well as any one or more of: (a) preventing a disorder from occurring in a subject who may be predisposed to the disorder but has not yet been diagnosed with it; (b) inhibiting a disorder, i.e. arresting its development; (c) relieving a disorder, i.e., causing regression thereof; (d) protection from or relief of a symptom or pathology caused by or related to a disorder; (e) reduction, decrease, inhibition, amelioration, or prevention of onset, severity, duration, progression, frequency or probability of one or more symptoms or pathologies associated with a disorder; and (f) prevention or inhibition of a worsening or progression of symptoms or pathologies associated with a disorder or comorbid with a disorder. Other such measurements, benefits, and surrogate or clinical endpoints, alone or in combination, will be known based on the teachings herein and knowledge in the art.

In embodiments, a patient will participate in a method of the invention, or be administered a disclosed entactogenic combination (herein, also as shorthand “an entactogenic combination” of the invention) as part of such a method, if the patient meets certain specified inclusion criteria, does not meet certain specified exclusion criteria, does not meet any specified withdrawal criteria during the course of treatment, and otherwise satisfies the requirements of the particular embodiment as claimed.

Entactogen-assisted psychotherapy (EAP) will be recognized as being useful for numerous conditions and disorders, and in embodiments, the disclosed entactogenic combinations are administered together with one or more sessions of psychotherapy or psychological support, such as one drug-assisted session, two drug-assisted sessions, three drug-assisted sessions, or more than three drug-assisted sessions, plus a number of preparation and/or integration sessions, for instance between one and three, between one and five, between one and ten, or an open-ended amount, and/or a number of follow-up or “check-in” or “progress update” sessions for ongoing maintenance and care, for instance between one and three, between one and five, between one and ten, or an open-ended amount. In embodiments, the disclosed entactogenic combinations are administered together with monitoring.

In embodiments, the disclosed entactogenic combinations are administered together with one or more therapeutically beneficial activities, where such participation follows or is in conjunction with the administration, including breathwork and breathing exercises, meditation and concentration practices, focusing on an object or mantra, listening to music, physical exercise, yoga, stretching or bodywork, journaling, grounding techniques, positive self-talk, or engaging with a pet or animal, and it should be understood that such participation can occur with or without the participation or guidance of a therapist or other professional, or with a friend. In other embodiments, the disclosed entactogenic combinations are administered alone as a single dose or a course of pharmacotherapy without psychotherapy or psychological support.

For example, EAP using a method known to those of skill, pharmacotherapy alone, pharmacotherapy with a beneficial activity, or pharmacotherapy with monitoring, using the entactogenic combination disclosed herein, will be recognized as useful for treating the disorders in the Diagnostic and Statistical Manual of Mental Disorders, 5th ed. (DSM-5). Indeed, patients with psychiatric or mental health conditions generally will benefit from the EAP of the invention. This conclusion is not limited to disorders defined by the DSM-5; EAP using the entactogen compositions of the invention will be appreciated as providing benefits to related disorders included in the International Classification of Diseases (ICD, including ICD-10 and ICD-11) and other diagnostic systems (e.g., a Merck Manual, such as The Merck Manual of Diagnosis and Therapy). In dimensional systems of personality, the therapeutic effects of the disclosed entactogenic combinations can be understood in several ways.

From a five-factor personality perspective, EAP appears to decrease neuroticism and increase openness, which can support positive life changes, self-acceptance, and other elements of wellness and therapeutic progress. From the Research Domain Criteria (RDoC) perspective, EAP decreases activity of negative valence systems and improves functioning of systems for social processes, which has secondary positive effects on positive valence, arousal, and cognitive systems.

Numerous separate and specific lines of reasoning are provided below, and numerous references to literature regarding use of entactogens are provided; however, it will be readily appreciated that such reasoning and references, while making application readily understandable to a reader for the disorders explicitly set forth, and using the evidence cited, also will apply to other disorders, and their absence in the descriptive section of another disorder is not meant to imply otherwise. General lines of reasoning, such as that alterations in consciousness and perception brought about by administration of an entactogenic combination may encourage a patient to view a disorder in a new way, and that such alterations or entactogenic mindstates may in embodiments be used to advantage in EAP, will be understood to apply to all relevant disorders.

In embodiments, a disclosed entactogenic combination is useful to treat any one or more of Disruptive Mood Dysregulation Disorder, Major Depressive Disorder (MDD), Treatment Resistant Depression, Persistent Depressive Disorder (Dysthymia), Premenstrual Dysphoric Disorder, Substance/Medication-Induced Depressive Disorder, Post-Partum depression, or Depressive Disorder due to Another Medical Condition, Separation Anxiety Disorder, Selective Mutism, Specific Phobia, Social Anxiety Disorder (Social Phobia), Panic Disorder, Panic Attack, Agoraphobia, Generalized Anxiety Disorder, Substance-Medication-Induced Anxiety Disorder, Anxiety Disorder Due to Another Medical Condition, Somatic Symptom Disorder, Illness Anxiety Disorder (hypochondriac), Conversion Disorder (Functional Neurological Symptom Disorder), Factitious Disorder, Post-Traumatic Stress Disorder (PTSD), Adjustment Disorders, Acute Distress Disorder, Obsessive-Compulsive Disorder, Body Dysmorphic Disorder, Hoarding Disorder, Trichotillomania (Hair-Pulling) Disorder, Excoriation (Skin-Picking) Disorder, Substance/Medication-Induced Obsessive-Compulsive and Related Disorder, Obsessive-Compulsive and Related Disorder due to Another Medical Condition, Substance-Related Disorders, Alcohol-Related Disorders, Cannabis-Related Disorders, Hallucinogen-Related Disorders, Inhalant-Related Disorders, Cocaine-Related Disorders, Opioid-Related Disorders, Sedative-, Hypnotic-, or Anxiolytic-Related Disorders, Stimulant-Related Disorders, Tobacco-Related Disorders, Non-Substance-Related Disorders (Gambling or Gaming Disorder), Migraines, Cluster Headaches such as Chronic Cluster Headaches, Cyclical Vomiting, Tension-Type Headache, Dysphasia, Pica, Anorexia Nervosa, Bulimia Nervosa, Binge-Eating Disorder, Oppositional Defiant Disorder, Intermittent Explosive Disorder, Conduct Disorder, Antisocial Personality Disorder, Psychopathy, Pyromania, and Kleptomania.

In some embodiments, disclosed combinations are useful to treat a central nervous system (CNS) disorder selected from the group consisting of: post-traumatic stress disorder (PTSD), major depressive disorder (MDD), treatment-resistant depression (TRD), suicidal ideation, suicidal behavior, major depressive disorder with suicidal ideation or suicidal behavior, non-suicidal self-injury disorder (NSSID), bipolar and related disorders including bipolar I disorder, bipolar II disorder, cyclothymic disorder, obsessive-compulsive disorder (OCD), generalized anxiety disorder (GAD), social anxiety disorder, substance use disorders including alcohol use disorder, opioid use disorder, amphetamine use disorder, nicotine use disorder, and cocaine use disorder, anorexia nervosa, bulimia nervosa, binge eating disorder, Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, sexual dysfunction, chronic fatigue syndrome, Lyme disease, and obesity.

In embodiments, disclosed combinations are useful to treat any one or more of a mental, behavioral and neurodevelopmental disorder, such as Anxiety or fear-related disorders; Catatonia; Disorders of bodily distress or bodily experience; Disorders due to substance use or addictive behaviors; Disorders specifically associated with stress; Disruptive behavior or dissocial disorders; Dissociative disorders; Elimination disorders; Factitious disorders; Feeding or eating disorders; Impulse control disorders; Mental or behavioral disorders associated with pregnancy, childbirth and the puerperium; Mood disorders; Neurocognitive disorders; Neurodevelopmental disorders; Obsessive-compulsive or related disorders; Paraphilic disorders; Personality disorders and related traits; and Schizophrenia or other primary psychotic disorders.

In embodiments, one or more signs or symptoms of a disorder is alleviated after administration of an entactogenic combination. In embodiments, at least one sign or symptom of a disorder is alleviated within 24 hours of administration of the entactogenic combination. In embodiments, at least one symptom of a disorder is alleviated within 1 week of administration of the entactogenic combination. In embodiments, at least one symptom of a disorder is alleviated for a period of at least 1 month after administration of the entactogenic combination. In embodiments, at least one symptom of a disorder is alleviated for a period of at least 3 months after administration of the entactogenic combination. In embodiments, at least one symptom of a disorder is alleviated for a period of at least 12 months after administration of the entactogenic combination. The signs and symptoms of a disorder will be known to those of ordinary skill.

By way of non-limiting and merely suggestive example, the following disorders will be treated in embodiments, whether by drug administration together with psychotherapy, drug administration together with psychological support, drug administration together with monitoring, drug administration together with an activity performed by the patient, and/or drug administration alone, by one or more of the methods discussed herein or as otherwise applicable and claimed.

a. Neurodevelopmental Disorders

In some embodiments, disclosed combinations are useful to treat neurodevelopmental disorders through at least two mechanisms. First, in embodiments, the entactogenic combination will increase neuroplasticity. Second, in embodiments, the entactogenic combination will decrease self-criticism and anxiety that impair neurocognitive functioning. Entactogens may induce a childlike-state of neuroplasticity, in which a person can relearn new behaviors and unlearn old ones. Mechanisms of entactogen-induced neuroplasticity may include increased expression of BNDF (Mattson, 2004).

Intellectual Disabilities: In some embodiments, disclosed combinations are useful to treat individuals with intellectual disability. Intellectual disability involves problems with general mental abilities that primarily affect two areas: intellectual and adaptive functioning. The former is benefited by the effects of entactogens on neuroplasticity and by EAP-related reductions in self-criticism and negative affect that worsen intellectual performance. The latter includes areas responsive to EAP, such as empathy, communication, social judgment, and the ability to make and keep friendships. The potential neuroadaptive properties of entactogens will also be useful in the treatment of other various intellectual disabilities, non-limiting examples of which include intellectual developmental disorder and global developmental delay.

Communication Disorders: In some embodiments, disclosed combinations are useful to treat communication disorders, such as Language Disorder, Social (Pragmatic) Communication Disorder, and Unspecified Communication Disorder. In embodiments, an entactogenic combination will promote a sense of intimacy with others, openness, and trust, all of which contribute to enhanced social cognition, and a vital component of the social interactions present in everyday life including but not limited to an improvement in communication in social settings by reducing social awkwardness (Schmid, 2014; Baggott, 2015).

Perhaps the first reported therapeutic effect of MDMA was a graduate student who achieved dramatic improvements in a stutter after receiving MDMA (Shulgin & Shulgin, 1991). Since then, other case studies have been documented where individuals have been able to speak fluently for several hours after taking an entactogen (Alm, 2004). In embodiments, entactogenic combinations are useful to treat Speech Sound Disorder (previously Phonological Disorder) and Childhood-Onset Fluency Disorder (Stuttering).

Autism Spectrum Disorder. In some embodiments, disclosed combinations are useful to treat individuals with autism spectrum disorder due to their anxiolytic effects. Autistic adults with moderate to extremely severe social anxiety who receive EAP or an entactogenic combination will experience a significant improvement in Liebowitz Social Anxiety Scale (LSAS) scores from baseline to primary endpoint. Following the completion of treatment, the reduction in social anxiety will either remain consistent or continue to improve, as has previously been demonstrated (Danforth, 2018). A systematic review of MDMA as a medicament in autistic social impairment found it to be efficacious as a pharmacological treatment (Chaliha, 2021), and the combinations herein are expected to have equal or greater efficacy, and in some embodiments, fewer adverse events.

Attention-Deficit/Hyperactivity Disorder, Other Specified and Unspecified Attention-Deficit/Hyperactivity Disorders (ADHD): In some embodiments, disclosed combinations are useful to reduce the signs and symptoms of ADHD, and treat individuals with ADHD.

Specific Learning Disorder. In some embodiments, disclosed combinations are useful to treat Specific Learning Disorder by, e.g., promoting neuroplasticity in the brain and increasing the propensity for social-reward learning (Nardou, 2019). The ability to learn new prosocial behaviors in adulthood, brought on by the administration of the entactogenic combination, will benefit individuals diagnosed with specific learning disorders, or learning disabilities, both of which are traditionally difficult to treat.

Motor and Tic Disorders: In some embodiments, disclosed combinations are useful to treat motor and tic disorders by, for example, reducing anxiety and stress. The frequency of involuntary motor movements and tics is often correlated with anxiety and stress, which an entactogenic combination will reduce, given its anxiolytic properties (Wolfson, 2020). Thus, in embodiments, the entactogenic combination is administered according to the disclosed methods, and optionally in combination with EAP, which engenders self-compassion and acceptance, and benefits individuals living with motor and tic disorders.

b. Schizophrenia Spectrum and Other Psychotic Disorders

Schizophrenia Spectrum Disorders, Schizophreniform Disorder, and Schizoaffective Disorder. In some embodiments, disclosed combinations are useful to treat schizophrenia and related disorders. For example, maladaptive social behavior commonly occurs in schizophrenia and other related neuropsychiatric disorders. In embodiments, the entactogenic combination will increase compassion for self and others, reduce psychological defenses and fear of emotional injury, and enhance communication and capacity for introspection (Holland, 2001; Heifets, 2016).

Schizoaffective disorder: In some embodiments, disclosed combinations are useful to treat schizoaffective disorder by targeting schizophrenia signs and symptoms, including but not limited to paranoia, agitation, compulsivity, excitability, hostility, delusion, confusion, apathy, and detachment, in addition to depression, a common comorbidity (Mayo Clinic, 2019). The entactogenic combination can target behavioral components affected by schizophrenia, and evidence suggests both depression and treatment resistant depression can benefit substantially from treatment with an entactogen such as that of the invention (Patel, 2015).

Schizotypal (Personality) Disorder. In some embodiments, disclosed combinations are useful to treat schizotypal personality disorder, which is typified by social anxiety and discomfort with others. Generally, impaired startle prepulse inhibition and habituation are found in patients with schizophrenia and schizotypal personality disorder (Bolino, 1994; Braff, 1992; Geyer, 1982; Cadenhead, 1993). In some embodiments, the entactogenic combination will acutely increase prepulse inhibition, and act as an efficacious treatment option (Liechti, 2001).

Psychotic Disorders: In some embodiments, disclosed combinations are useful to treat psychotic disorders by encouraging prosocial behavior and enhancing communication. In some embodiments, the entactogenic combination will lessen defenses, reduce paranoia, promote openness and trust, and increase subjective insight (Holland, 2000). In some embodiments, the entactogenic combination will reduce the suffering and alienation experienced by an individual including in some embodiments, the individual's family member(s), who participate together in a guided entactogenic-dosing session that provides a therapeutic environment in which trust, intimate communication, sensitivity to the other's perspective and feelings, and a reduction in paranoia are achieved, such as through the entactogenic combinations as administered according to the disclosed methods, and EAP, as practiced in the disclosed methods.

Substance/Medication-Induced Psychotic Disorder. In some embodiments, disclosed combinations are useful to treat substance/medication-induced psychotic disorder, for instance where the entactogenic combination is administered with a therapy session.

Delusional Disorder, Brief Psychotic Disorder, and Psychotic Disorder Due to a Medical Condition: In some embodiments, disclosed combinations are useful to treat other psychotic disorders caused by a medical condition, alienation, or inhibited communication and characterized by the occurrence of delusions/hallucinations, including but not limited to, delusional disorder, brief psychotic disorder, and psychotic disorder, due to their therapeutic effects on anxiety, and ability to ease hostility.

Catatonia and Catatonia Due to a Medical Condition: In some embodiments, disclosed combinations are useful to treat catatonia due to its promotion of prosocial behavior. In embodiments, the entactogenic combination will increase prosocial behavior and the content of social and emotional communication (Baggott et al., 2015; Wunderli et al., 2018).

c. Bipolar and Related Disorders

In some embodiments, disclosed combinations are useful to treat bipolar and related disorders, and provides effects comparable to an anxiolytic, while also helping to regulate emotion. Many patients diagnosed with bipolar disorder II, bipolar II, depressive episodes with short-duration hypomania, and related bipolar disorders, experience anxiety or substance use disorders as a comorbidity (McElroy, 2004). An entactogenic combination is useful to treat such disorders, e.g., because of its ability to improve emotional regulation (Johansen and Krebs, 2009), and to allow patients to explore and address painful memories without being overwhelmed by negative affect (Sessa, 2018).

Cyclothymic Disorder. In some embodiments, disclosed combinations are useful to treat cyclothymic disorder, which is characterized by intermittent “highs” and “lows,” as it pertains to mood. The entactogenic combination will provide improvements in mood stabilization (Patel et al., 2015; Passie et al., 2005), and will be useful in treating individuals experiencing emotional fluctuations.

d. Depressive Disorders

Depression broadly encompasses major depressive disorder (MDD), treatment resistant depression (TRD), bipolar depression, persistent depressive disorder (PDD), seasonal affective disorder (SAD), peripartum (postpartum) depression, atypical depression, and other forms of depression known to one of skill in treating such disorders. While depression is different for all encumbered with the disease, the symptoms are consistent across the varying forms, comprising loss of interest or pleasure in one's activities, weight loss or weight gain, trouble sleeping, or feeling drowsy during the day; feeling restless and agitated, or sluggish both physically and mentally; being tired and without energy, feeling worthless or guilty, trouble concentrating or making decisions, and, in some cases, thoughts of suicide (Pietrangelo, 2021).

Therapies that mitigate symptoms and treat the underlying cause of depression are desperately needed. In some embodiments, disclosed combinations and methods are effective for the treatment of depression, including major depressive disorder (MDD), treatment resistant depression (TRD), bipolar depression, persistent depressive disorder (PDD), seasonal affective disorder (SAD), peripartum (postpartum) depression, atypical depression, and other forms of depression known to one of skill.

In embodiments, the entactogenic combinations are administered alone. In embodiments, the entactogenic combinations are administered as adjunctive pharmacotherapy together with psychotherapy or other healing modalities such as disclosed herein or known to those of skill.

Given the entactogenic state brought about by administration of the disclosed entactogenic combinations, patients may be guided to identify, and adequately deal with, triggers causing depression. For example, EAP as disclosed will increase openness and significantly reduce neuroticism, which will alleviate signs and symptoms of depression and facilitate behavioral and cognitive change (Wagner et al., 2017).

In some embodiments, chronic suicidality pervasive in severe depression may respond well to treatment with disclosed compositions and combinations, which can produce enhanced insight and self-acceptance (Shannon, Colbert, and Hughes 2021).

In embodiments, the disclosed entactogenic combinations act as rapid-acting antidepressants, allowing them to augment cognitive and other therapy (Patel and Titheradge, 2015). In embodiments, the entactogenic combinations, with or without EAP, are useful to treat individuals with MDD, dysthymia, premenstrual dysphoric disorder, persistent complex bereavement disorder and other specified and unspecified depressive disorder, as well as drug-resistant and other treatment-resistant depression (TRD).

Substance/Medication-induced Depressive Disorder. In some embodiments, disclosed combinations are useful to treat such disorders by allowing patients to address painful memories without being overwhelmed by negative effects, through the entactogenic state it elicits (Sessa, 2018).

Disruptive Mood Dysregulation Disorder. In embodiments, an entactogenic combination will improve mood, build trust, and promote prosocial behavior, and is used to treat disruptive mood dysregulation disorder (Passie, 2012). A disclosed entactogenic combination therefore may be used to treat children with Disruptive Mood Dysregulation Disorder (DMDD) (Hysek et al., 2014).

e. Anxiety Disorders

In some embodiments, disclosed combinations are useful to treat anxiety disorders through, among other mechanisms and effects, enhancement of the therapeutic alliance; improved emotional regulation and decrease avoidance; and increased emotional engagement and enhanced extinction of learned fear associations (Johansen and Krebs, 2009). In embodiments, the entactogenic combination lowers fear and anxiety by putting a person in an extremely open frame of mind, with expanded capacity for self-awareness, expanded sensitivity, and an enhanced ability to share feelings (Ingrasci, 1985).

Panic Attack, Panic Disorder and Agoraphobia: In some embodiments, disclosed combinations are useful to treat panic attacks due to their fear-extinction qualities. Panic disorders, agoraphobia, and chronic panic-related anxiety benefit from its fear-extinction effects (Shannon, Colbert, and Hughes, 2021).

Anxiety Disorder Due to a Medical Condition: In some embodiments, disclosed combinations are useful to alleviate anxiety arising from life-threatening illnesses, for example because of anxiolytic effects, and promoting ability to confront the anxiety with self-compassion.

Generalized Anxiety Disorder and Substance/Medication-Induced Anxiety Disorder. In some embodiments, disclosed combinations are useful to alleviate anxiety arising from GAD and substance/medication use by improving emotional regulation, decreasing avoidance, and providing insights into thought patterns that prevent personal progress and well-being (Johansen and Krebs, 2009).

Social Anxiety Disorder (Social Phobia): In some embodiments, disclosed combinations are useful to treat social anxiety disorder by fostering trust, and promoting prosocial behavior (Wagner, 2017).

Separation Anxiety Disorder and Selective Mutism: In some embodiments, disclosed combinations are useful to treat separation anxiety disorder as well as selective mutism due to therapeutic effects on fear, as well as the ability to improve emotional regulation and decrease avoidance (Johansen and Krebs, 2009). In embodiments, an entactogenic combination will help extinguish learned fear associations that children with separation anxiety may experience (Feduccia and Mithoefer, 2018). Children diagnosed with selective mutism may experience similar benefits due to enhanced prosocial behavior and ability to increase social and emotional communication content (Baggott, 2015).

Specific Phobia: In some embodiments, disclosed combinations are useful to treat specific phobias, for example by facilitating fear extinction (Young et al., 2015).

f. Trauma- and Stressor-Related Disorders

In some embodiments, disclosed combinations are useful to treat trauma and stressor-related disorders. In some embodiments, disclosed combinations are useful in treating post-traumatic stress disorder (PTSD). In some embodiments, disclosed combinations are also useful to treat various phobias, for example by diminishing fear- and anxiety-related behaviors, and allowing the reprocessing of traumatic or fear-inducing memories. In some embodiments, disclosed combinations are useful to treat acute stress disorder (ASD) (Jerome et al., 2020), for example by regulating emotional memory processing, which may help reduce distress in confronting traumatic memories, increasing emotional empathy for self and others, and increasing self-compassion, and by enhancing fear extinction. Such effects offer therapeutic benefits for acute stress disorder, and other specified and unspecified trauma and stressor-related disorders.

g. Obsessive-Compulsive and Related Disorders

In some embodiments, disclosed combinations are useful to improve aspects of decision-making and provide emotional clarity in healthy volunteers and, therefore, in embodiments, to alleviate anxiety, doubt, and rumination in people with obsessive-compulsive disorder (OCD) and related disorders. Additionally, the entactogenic combinations will produce openness, helping to facilitate the development of insights and attitudes often sought via psychotherapy, and may contribute to improved wellbeing and decreased stress, thus improving signs and symptoms of OCD and related disorders.

Obsessive-Compulsive Disorder, Substance/Medication-Induced Obsessive-Compulsive and Related Disorder Obsessive-Compulsive and Related Disorder Due to Another Medical Condition: In some embodiments, disclosed combinations are useful to treat diverse OCD disorders due to their effects and their ability to regulate the brain regions influencing OCD behavior (Sessa, 2010). In some embodiments, disclosed combinations are useful to increase prepulse inhibition (PPI). Both PPI and habituation deficits have been found in patients with obsessive-compulsive disorder.

Trichotillomania (Hair-Pulling Disorder), Excoriation (Skin-Picking) Disorder. In some embodiments, disclosed combinations are useful to treat trichotillomania, e.g., because of benefits to patients with comorbid disorders prevalent in those with trichotillomania, such as anxiety, depression, OCD, and PTSD, and by decreasing stress and anxiety and facilitating development of healthier coping behaviors.

Body Dysmorphic Disorder. In embodiments, disclosed combinations are useful to treat body dysmorphic disorder, including signs, symptoms, and comorbidities, for example because of a decrease in neuroticism and self-criticism and an increase in self-acceptance. In embodiments, the entactogenic combinations will decrease the effect of simulated social rejection on self-reported mood and self-esteem (Frye et al., 2014), and will modulate chemicals in the brain that produce positive feelings, especially serotonin. Substance use disorder (SUD) is also relatively common in individuals diagnosed with body dysmorphic disorder and can be seen as a way to cope with, or “escape” from, its signs and symptoms. In embodiments, disclosed combinations are useful to treat SUDs (Jerome, Schuster, and Klosinski, 2013).

Reactive Attachment Disorder (RAD) and Disinhibited Attachment Disorder (DAD): In some embodiments, disclosed combinations are useful to treat RAD and DAD and reduce the signs and symptoms thereof through promotion of prosocial behavior (Hysek et al., 2014), and enhancement of communication about emotional and social matters (Baggott et al., 2015). In embodiments, the entactogenic combinations aid in the treatment of trauma, which children diagnosed with RAD and DAD may experience, due to their difficulty in forming healthy bonds with their primary caregivers (Jerome et al., 2020).

h. Adjustment Disorders

In some embodiments, disclosed combinations are useful to ease psychological stress and help individuals cope with negative life events, which are especially difficult for those struggling with adjustment disorders (Moonzwe, Schensul, and Kostick, 2011). Those diagnosed with adjustment disorders commonly experience anxiety as a comorbidity and the entactogenic combinations will ease anxiety by putting a person in an open frame of mind having an expanded capacity for self-awareness and sensitivity, and an enhanced ability to share feelings (Ingrasci cited in Gertz, 1985).

i. Dissociative Disorders

In embodiments, disclosed combinations are useful to treat dissociative disorders by addressing the root causes, reducing hyperarousal, and modulating emotional memory circuits underlying the disorder.

Dissociative Identity Disorder. In some embodiments, disclosed combinations are useful to ease psychological stress and help individuals cope with negative life events, which are especially difficult for those living with dissociative identity disorders (Moonzwe, Schensul, and Kostick, 2011). Dissociative identity disorders often develop as a defensive mechanism in response to a traumatic experience to keep difficult memories at bay. In some embodiments, disclosed combinations are useful to treat trauma via mechanisms like enhanced emotional memory processing, greater self-compassion, and an enhancement of fear extinction. Neuroses also frequently accompany dissociative disorders (Jerome et al., 2020), and the entactogenic combinations increase openness and reduce neuroticism (Wagner et al., 2017).

Dissociative Amnesia: In some embodiments, disclosed combinations are useful to treat dissociative amnesia by helping to reconsolidate memory and in particular the reprocessing and reconsolidation of traumatic memories (Feduccia and Mithoefer, 2018).

Depersonalization/Derealization Disorder. Depersonalization and derealization both commonly occur in PTSD patients and are improved with EAP. In embodiments, disclosed combinations are useful to treat PTSD signs and symptoms, and will be useful to treat depersonalization/derealization disorder.

j. Somatic Symptom and Related Disorders

In some embodiments, disclosed combinations are useful to treat somatic symptom and related disorders by decreasing self-focused neuroticism and rumination and refocusing the individual on positive thoughts and behaviors.

Somatic Symptom Disorder. In some embodiments, disclosed combinations are useful to treat somatic symptom disorder by reducing self-referential thinking, enhancing feelings of sensuality, releasing muscular tension, and inducing sensations of analgesia (Oehen et al., 2013).

Illness Anxiety Disorder. In some embodiments, disclosed combinations are useful to treat anxiety and psychological distress related to illness, or due to a medical diagnosis (Wolfson et al., 2020).

Conversion Disorder (Functional Neurological Symptom Disorder): In some embodiments, disclosed combinations are useful to treat tremors present in Parkinson's patients, for example by stimulating the release of serotonin in the brain (Sotnikova et al., 2005).

Factitious Disorder. In some embodiments, disclosed combinations are useful to treat factitious disorder by allowing underlying emotional and attachment difficulties that predate and fuel this disorder to be addressed, as well as childhood trauma, recent trauma, depression, and personality disorder, and by generally improving poor self-esteem, which has been linked to factitious disorder (Frye et al., 2014).

k. Feeding and Eating Disorders

In some embodiments, disclosed combinations are useful to treat eating disorders. Entactogenic treatment may be effective for eating disorders, which for instance may resolve after insights achieved through the administration of entactogenic compounds in the absence of formal therapy (Zoe, 2014), or administered during a session or as part of a course of EAP.

In some embodiments, disclosed combinations are useful to treat eating disorders associated with PTSD, for example by: (1) reducing fear, (2) enhancing wellbeing, (3) increasing sociability/extraversion, (4) reducing self-criticism, (5) increasing compassion for self/others, (6) increasing interpersonal trust, and (7) facilitating an alert state of consciousness (Brewerton, Lafrance, and Mithoefer, 2021), features that also will benefit other disorders herein. The anxiolytic and prosocial effects associated with the entactogenic combinations will counteract avoidance and hyperarousal in the context of psychotherapy for those with eating disorders (EDs) linked to post-traumatic stress disorder (ED-PTSD). Other clinical features of EDs that will be amenable to the entactogenic combinations and EAP include body image distortion, cognitive rigidity, and socio-emotional processing difficulties. The wide range of therapeutic applications can aid in the treatment of diverse feeding and eating disorders.

Pica: The entactogenic combinations can help to ease psychological stress and anxiety and promote openness and communication, all of which benefits individuals diagnosed with pica (Moonzwe, Schensul, and Kostick, 2011). Individuals diagnosed with schizophrenia, obsessive-compulsive disorder (OCD), or autism are more likely to develop pica as a coping mechanism. Using the entactogenic combinations to address these conditions can alleviate pica. In embodiments, the entactogenic combinations are thus useful to treat patients with pica.

Rumination Disorder: In embodiments, disclosed combinations are useful to treat rumination disorder, which may be caused by a triggering event in a person's life causing stress and subsequent feeding problems. The entactogenic combinations will facilitate coping with negative life events as well as decrease anxiety and neuroticism that sustains rumination (Moonzwe, Schensul, and Kostick, 2011).

Avoidant/Restrictive Food Intake Disorder (ARFID): In some embodiments, disclosed combinations are useful to treat avoidant/restrictive food intake disorder (ARFID), as obsessive compulsive disorder (OCD) or anxiety are often mitigating factors contributing to ARFID, as are anxiety, depression, and depressive mood (Mûller et al., 2021; Passie, 2012)), all of which are treated by the disclosed combinations.

Anorexia Nervosa: In some embodiments, disclosed combinations are useful to treat anorexia nervosa by, as non-limiting examples, producing anxiolytic and prosocial effects, slowing identification of negative emotions, increasing acceptance of self and others, and increasing one's ability to address emotionally upsetting topics. The entactogenic combinations will facilitate emotional processing, like improving dysregulation and poor self-esteem, and will create a desirable psychological state that can also be used to enhance the therapeutic process.

Binge-Eating Disorder: In some embodiments, disclosed combinations are useful to treat many of the signs, symptoms, and comorbidities of binge-eating disorder (BED). BED is linked to issues such as poor self-esteem/self-image and trauma, but may also occur as a comorbidity of disorders like anxiety, depression, bipolar disorders, substance abuse disorder, etc., in which the entactogenic combinations will aid in treatment. In addition, the entactogenic combinations will promote enhanced personal acceptance and forgiveness (Shannon, Colbert, and Hughes, 2021), and thereby aid in the treatment of BED.

Bulimia Nervosa: In some embodiments, disclosed combinations are useful to treat bulimia, by enhancing personal acceptance and forgiveness, and treating the profound sense of shame and loss of personal control often associated with the disorder (Shannon, Colbert, and Hughes, 2021).

Elimination Disorders: In some embodiments, disclosed combinations are useful to treat disorders that occur as comorbidities of elimination disorders and that contribute to the severity of elimination disorders. In children, enuresis, encopresis and other specified and unspecified elimination disorders can be triggered by emotional stressors or risk factors such as ADHD, ASD, or anxiety. Elimination disorders will be treated through the enhanced personal acceptance, fear elimination, and forgiveness associated with administration of entactogenic combinations.

l. Sleep-Wake Disorders

Insomnia Disorder: In some embodiments, disclosed combinations are useful to treat many disorders that lead or contribute to insomnia, including but not limited to stress, anxiety disorders, and depression. The entactogenic combinations will ease psychological stress and aid in coping with negative life events that contribute to sleeplessness (Moonzwe, Schensul, and Kostick, 2011).

Hypersomnolence Disorder and Narcolepsy: In some embodiments, disclosed combinations are useful to treat comorbidities, such as SUDs, depression, and bipolar disorder, which have been linked to hypersomnolence disorder, and without producing daytime sleepiness (Randall et al., 2009).

Breathing-Related Sleep Disorders: In some embodiments, disclosed combinations are useful to treat conditions linked to breathing-related sleep disorders. Studies show an association between breathing-related sleep disorders such as obstructive sleep apnea hypopnea, central sleep apnea, sleep-related hypoventilation, circadian rhythm sleep-wake disorders, and mood, anxiety, and PTSD. The anxiolytic effects of the entactogenic combinations will be long-lasting, easing anxiety and fear-response when treating such disorders (Wolfson et al., 2020).

Non-Rapid Eye Movement Sleep Arousal Disorders: In some embodiments, disclosed combinations are useful to treat the signs, symptoms, and causes of non-rapid eye movement sleep arousal disorders (NREM). These disorders have been linked to physical or emotional stress, among other triggers. The entactogenic combinations will ease psychological stress and release muscular tension, leading to durable improvements (Moonzwe, Schensul, and Kostick, 2011; Oehen et al., 2013).

Sleep Terrors and Nightmare Disorder: In some embodiments, disclosed combinations are useful to treat the signs, symptoms, and causes of sleep terrors and nightmare disorder. Sleep terrors are very commonly experienced by individuals diagnosed with PTSD. The entactogenic combination will have effects on emotional memory processing which may reduce distress when facing traumatic memories, an increase in emotional empathy for self and others, and enhancement of fear extinction, thus having utility in treating sleep terrors (Jerome et al., 2020). In embodiments, the entactogenic combinations are also useful to treat individuals diagnosed with nightmare disorder by aiding uninterrupted sleep and enhanced wellbeing.

Sleepwalking: In some embodiments, disclosed combinations are useful to treat sleepwalking.

REM Sleep Behavior Disorder, Substance/Medication-Induced Sleep Disorder: In some embodiments, disclosed combinations are useful to treat sleep behavior disorders, and to treat substance- or medication-induced sleep disorders, including both independently and by treating an underlying substance abuse disorder with which a substance-induced sleep disorder is comorbid.

m. Sexual Dysfunctions

In some embodiments, disclosed combinations are useful to treat sexual dysfunctions. The entactogenic combinations will, in embodiments, produce euphoria, enhance interpersonal communication, and promote feelings of closeness with others, offering a range of benefits for individuals diagnosed with both specified and unspecified sexual dysfunctions (Passie et al., 2005).

Delayed Ejaculation and Erectile Disorder: In embodiments, an entactogenic combination will enhance feelings of sensuality and release muscular tension, both of which may inhibit delayed ejaculation disorder or erectile disorder (Oehen et al., 2013). Both disorders have also been linked to depression, anxiety, communication, and intimacy problems, as well as anxiety concerning sexual performance which the entactogenic combinations will treat by, for example, easing psychological stress and enhancing openness, trust, and increasing willingness to disclose (Wagner et al., 2017).

Female Orgasmic Disorder, Female Sexual Interest/Arousal Disorder, Genito-Pelvic Pain/Penetration Disorder: In some embodiments, disclosed combinations are useful to treat anxiety, depression, poor self-esteem, poor body image, and past trauma, which have been linked to sexual disorders in women. The entactogenic combinations will be effective anxiolytics and will possess benefits for individuals experiencing trauma by promoting self-acceptance and compassion for self and others (Kamboj et al., 2015), and, e.g., helping to process and heal from trauma associated with a sexual assault.

Male Hypoactive Sexual Desire Disorder: In embodiments, disclosed combinations are useful to treat men diagnosed with male hypoactive sexual desire disorder (MHSDD) by enhancing sexual desire and increasing sexual satisfaction. The entactogenic combinations will also address issues linked to HSDD such as anxiety, depression, poor self image, or trauma (Zemishlany, Aizenberg, and Weizman, 2001).

Premature (Early) Ejaculation (PE): In some embodiments, disclosed combinations are useful to aid in improving PE by decreasing self-criticism and anxiety and allowing individuals to more fully enjoy their sexuality and achieve satisfying emotional closeness with their partners. In embodiments, coupled with the intimacy experienced after administration of an entactogenic combination, will be useful to treat PE, and orgasm/ejaculation may also be delayed but perceived as more intense and pleasurable during the acute effects of an entactogenic combination (Zemishlany, Aizenberg, and Weizman, 2001; Passie et al., 2005).

Substance/Medication-Induced Sexual Dysfunction: In some embodiments, disclosed combinations are useful to treat substance abuse disorder, while also addressing comorbid sexual dysfunctions arising from substance abuse (Jerome, Schuster, and Klosinski, 2013).

Gender Dysphoria: In some embodiments, disclosed combinations are useful to treat forms of gender dysphoria via a plurality of mechanisms, a non-limiting example being allowing an individual to experience positive affirmation of themselves, by increasing self-compassion and unconditional self-love (Sevelius, 2019). For transgender and gender-diverse people who perceive themselves as outsiders, the entactogenic combinations will provide a pathway for reconnecting with themselves and others.

n. Disruptive, Impulse-Control, and Conduct Disorders

Oppositional Defiant Disorder, Intermittent Explosive Disorder, Conduct Disorder, and Antisocial Personality Disorder: In some embodiments, disclosed combinations are useful to treat disruptive, impulse-control, and conduct disorders characterized by antisocial elements. An entactogenic combination will give a patient improved self-knowledge, greater accuracy in perceiving the mental states of others, enhanced coping strategies, emotional regulation, cognitive insights, emotional empathy, and compassion for self and others, and will lead to increased prosocial behavior (Hysek et al., 2014).

Pyromania and Kleptomania: In some embodiments, disclosed combinations are useful to treat pyromania and kleptomania due to their ability to increase impulse control (Ramaekers and Kuypers, 2006). Impulse control disorders are also commonly linked to a diagnosis of another mental health condition such as conduct disorder, trauma, substance abuse disorder, or a deficit in social skills, which can all benefit from administration of a disclosed entactogenic combination.

a. Substance-Related and Addictive Disorders

In some embodiments, disclosed combinations are useful to treat substance-related and other addictive disorders, including gambling disorder, internet gaming disorder, and non-suicidal self-injury disorder, due to their influences on perception of self, others, habits, and consciousness. The entactogenic combinations will, in embodiments, directly treat neuropharmacological abnormalities associated with addiction, indirectly assist with the therapeutic process, and/or reduce signs and symptoms of comorbid psychiatric conditions, thus providing an enhanced opportunity to address problematic substance use (Jerome, Schuster, and Klosinski, 2013). In some embodiments, disclosed combinations are useful in treating such disorders by exerting serotonergic activity.

In embodiments, the entactogenic combinations will alter consciousness in ways that lead patients to view their life or behavior in new ways (Müller et al., 2021). Changes to social cognition, interpersonal closeness, and communication brought on by administration of the entactogenic combinations can influence the outcome of treatments for substance abuse disorder and comorbid psychological disorders.

Substance-related Disorders: In some embodiments, disclosed combinations are useful to treat substance-related disorders, substance use disorders (SUDs) and addictions. The entactogenic combinations will elicit feelings of openness, empathy, sympathy, relatedness, etc., which enables a patient to more readily confront the underlying causes of substance abuse. Substance abuse and misuse often has a genesis rooted in traumatic experiences, with the substance used as a coping mechanism. By administering to a patient ab effective dose of an entactogenic combination, the entactogenic effects will be useful to help come to terms with the traumatic events that were a precursor to substance misuse, and like reasoning applies to other disorders with traumatic underpinnings, such as those disclosed herein.

In some embodiments, disclosed combinations are useful to support the treatment of substance-related disorders by fostering greater interpersonal awareness and increasing empathy, permitting a patient to acknowledge the impact their substance use has on themselves and others (Jerome et al., 2013). Increased self-awareness and empathy may reduce or attenuate the individual's denial of substance abuse, removing a significant hurdle to successful treatment. Increased empathy and a reduction of denial may support a greater motivation to change behavior. In embodiments, the entactogenic combinations enhance and intensify the psychotherapeutic processes utilized in the treatment of substance misuse, such as for alcohol, cannabis, caffeine, tobacco, cocaine, or others (Greer and Tolbert, 1986).

Alcohol-related Disorders: In embodiments, the entactogenic combinations will be useful to treat alcohol-use disorders, e.g., by providing an opportunity to reflect on and address problematic alcohol use, in a mindstate facilitating self-compassion and self-forgiveness (Sessa, 2018; Greer and Tolbert, 1986).

Caffeine-related Disorders: In embodiments, the entactogenic combinations will be useful to treat neuropharmacological abnormalities associated with addiction, and are effective as a treatment for caffeine addiction, caffeine withdrawal, and other caffeine related disorders (Jerome, Schuster, and Klosinski, 2013), e.g., by providing an opportunity to reflect on and address problematic caffeine use, in a mindstate facilitating self-compassion and self-forgiveness.

Cannabis-related Disorders: In embodiments, the entactogenic combinations will be useful to treat cannabis addiction, cannabis use disorder, and other cannabis-related disorders, e.g., by providing an opportunity to reflect on and address problematic cannabis use, in a mindstate facilitating self-compassion and self-forgiveness (Tourino, Maldonado, and Valverde, 2007).

Phencyclidine-related disorders: In embodiments, the entactogenic combinations will be used as an acutely-acting antipsychotic, which aids the treatment of phencyclidine-related disorders, as persisting hallucinogenic effects from phencyclidines can place the individual into a psychotic state (Holland, 2000). In embodiments, the entactogenic combinations will be useful to treat somatic signs and symptoms associated with phencyclidine-related disorders by enhancing feelings of sensuality, releasing muscular tension, and inducing sensations of analgesia (Oehen et al., 2013), feelings that help to consolidate a sense of self and reduce somatic symptoms.

Hallucinogen-related Disorders, Inhalant-related Disorders and Opioid-related Disorders: In embodiments, the entactogenic combinations will be useful to treat hallucinogen, inhalant, or opioid-related disorders, for instance by altering consciousness and leading patients to view their life or behavior in new ways (Müller et al., 2021). Entactogenic combinations in embodiments assist with the therapeutic process by opening an opportunity to address problematic hallucinogen, inhalant, or opioid use (Jerome et al., 2013). Increasing empathy for self and others allows patients to acknowledge the impact their disorder has on themselves and others, providing motivation to change the behavior. Increased self-awareness will promote acceptance of addiction and launch a patient on a path to treatment.

Sedative-, Hypnotic-, or Anxiolytic-Related Disorders: In embodiments, the entactogenic combinations will be useful to treat sedative, hypnotic or anxiolytic-related disorders by lowering fear and anxiety. The entactogenic combinations will place a person in an open frame of mind with expanded capacity for self-awareness and sensitivity, enhancing the extinction of learned fear-associations (Johansen and Krebs, 2009). Such benefits can remove the need for sedative, hypnotic, or anxiolytic medications and use disorders arising from them.

Stimulant-Related Disorders: In embodiments, the entactogenic combinations will be useful to treat stimulant-related disorders. Unlike common treatments, the entactogenic combinations produce increased emotional openness and decreased neuroticism that can facilitate behavioral change such as, in embodiments, during a drug-administration session of EAP or during an integration session of EAP after administration of a disclosed combination or composition.

Tobacco-Related Disorders: In some embodiments, disclosed combinations are useful to treat tobacco-related disorders. 5-HT_{2A} receptor agents, like certain entactogenic combinations, will be effective in the treatment of tobacco-related disorders (Johnson et al., 2014), and will also provide an opportunity to reflect on and address problematic tobacco use, e.g., for reasons above.

o. Neurocognitive Disorders

In some embodiments, disclosed combinations are useful to treat neurocognitive disorders by encouraging neuroplasticity, neuroadaptation, improved emotional regulation, and cognitive improvements. Entactogenic combinations will promote neuroplasticity (Mattson, Maudsley, and Martin, 2004; Yazar-Klosinski and Mithoefer, 2017), and may promote neuroplasticity by creating brain conditions such as seen during adolescence (Nardou et al., 2019; Edut et al., 2011).

Delirium: In some embodiments, disclosed combinations are useful to treat delirium. Anxiety generally exacerbates the signs and symptoms of delirium, and thus entactogenic combinations having anxiolytic properties will offer a useful treatment (Lin et al., 1999).

Major or Mild Neurocognitive Disorder Due to Traumatic Brain Injury (TBI): In embodiments, disclosed combinations are useful to treat major or mild neurocognitive disorder caused by TBI, for example by its effects on visual and spatial memory, such as by minimizing their alteration (Harvey, 2003).

p. Personality Disorders

In some embodiments, disclosed combinations are useful to treat a wide range of personality disorders due to their prosocial and empathy increasing mechanisms, and by encouraging openness, enhancing trust and intimacy, and minimizing neurotic filters that arise as a result of a patient's trauma and attachment patterns (Hysek et al., 2014; Shannon, Colbert, and Hughes, 2021). Such benefits will allow patients to perceive and communicate with greater accuracy, which may additionally expedite psychotherapy in embodiments including EAP.

Cluster A Personality Disorders: In embodiments, disclosed combinations are useful to treat Cluster A personality disorders including paranoid personality disorder, schizoid personality disorder, and schizotypal personality disorder, due to its prosocial and anxiolytic effects. An entactogenic combination will treat social anxiety, a hallmark of the such disorders, by increasing sociability towards others and enhancing responses to positive social stimuli following use (Danforth et al., 2018; Kamilar-Britt and Bedi, 2015).

Cluster B Personality Disorders: In some embodiments, disclosed combinations are useful to treat Cluster B personality disorders, which are often characterized by dramatic, over-emotional antisocial behavior. By increasing empathy, patients will develop self-awareness about the effects their behavior has on others. With respect to communication, an entactogenic combination will promote willingness to disclose and increase the use of social words, and enhance positive emotional memories (Baggott et al., 2015).

Cluster C Personality Disorders: In some embodiments, disclosed combinations are useful to treat Cluster C personality disorders such as avoidant personality disorder, dependent personality disorder and obsessive-compulsive personality disorder that are characterized by anxious, fearful thinking and behavior. The entactogenic combinations will lower fear and anxiety by opening up an individual's frame of mind, expanding their capacity for self-awareness and reflection, and enhancing the extinction of learned fear-associations (Johansen and Krebs, 2009).

Paraphilic Disorders: In some embodiments, disclosed combinations are useful to treat paraphilic disorders by targeting both the comorbidities and causes of the disorder. Individuals diagnosed with paraphilic disorders often experience comorbidities such as various personality disorders, anxiety disorders, poor-self image, or may have experienced trauma or abuse as a child, all of which can benefit from administration of the entactogenic combinations. For example, in embodiments the entactogenic combinations will act as an effective anxiolytic, promote self-acceptance and compassion for self and others (Kamboj et al., 2015), and decrease the effects of simulated social rejection on self-reported mood and self-esteem (Frye et al., 2014). In embodiments, the entactogenic combinations will reduce fear and allow a patient to work through sexual trauma, and result in a decrease of symptoms such as anxiety, hyperarousal, and depression. In embodiments, the entactogenic combinations, including their administration as part of EAP, will be useful to treat paraphilic disorders such as voyeuristic disorder, exhibitionistic disorder, frotteuristic disorder, sexual masochism disorder, sexual sadism disorder, pedophilic disorder, fetishistic disorder, transvestic disorder, and other specified and unspecified paraphilic disorders

q. Neurodegenerative, Pain, and Inflammatory Disorders

In some embodiments, the medical condition is a neurodegenerative disorder. In some embodiments, the neurodegenerative disorder is selected from the group consisting of Alzheimer's disease (AD), corticobasal degeneration (CBD), a form of dementia, Huntington's disease, Lytico-Bodig disease, mild cognitive impairment (MCI), a motor neuron disease, progressive supranuclear palsy (PSP), multiple sclerosis, Parkinson's disease, and traumatic brain injury (TBI). In some embodiments, the medical condition is pain and/or a pain disorder. In some embodiments, the pain disorder is selected from the group consisting of arthritis, allodynia, atypical trigeminal neuralgia, trigeminal neuralgia, somatoform disorder, hypoesthesia, hyperalgesia, neuralgia, neuritis, neurogenic pain, phantom limb pain, analgesia, anesthesia dolorosa, causalgia, sciatic nerve pain disorder, degenerative joint disorder, fibromyalgia, visceral disease, chronic pain disorders, headache disorders, migraine headaches, chronic cluster headaches, concussion headache, short-lasting unilateral neuralgiform headache attacks, chronic fatigue syndrome, complex regional pain syndrome, neurodystrophy, plantar fasciitis, or pain associated with cancer. In some embodiments, the medical condition is inflammation and/or an inflammatory disorder. In some embodiments, the inflammatory disorder is characterized by any one or more of skin inflammation, muscle inflammation, tendon inflammation, ligament inflammation, bone inflammation, cartilage inflammation, lung inflammation, heart inflammation, liver inflammation, pancreatic inflammation, kidney inflammation, bladder inflammation, gastric inflammation, intestinal inflammation, neuroinflammation, and brain inflammation.

r. Improving Mental Health and Functioning

In some embodiments, disclosed combinations are useful to provide an improvement in mental health and functioning. In some embodiments, an improvement in mental health and functioning is an improvement in creativity. Broadly, and without being bound by theory, four categories of creativity are defined: deliberate cognitive, deliberate emotional, spontaneous cognitive, and spontaneous emotional (Shortle, 2016). Specifically, deliberate cognitive creativity, the driver of which is the prefrontal cortex, is a mental process relying heavily on one's existing body of knowledge, and is something a person is consciously aware of. Meaning, one is able to consciously think about something and piece together known information to form an original idea. Deliberate cognitive creativity is largely invoked in problem-solving tasks (Shortle, 2016). Spontaneous cognitive creativity is an unconscious and unpredictable mental process, but also requires an existing body of knowledge. This often occurs when the conscious mind stops working on a problem, and the unconscious mind begins working on it instead. With one's mind working on the problem “in the background,” so to speak, one merely needs to see something that triggers an organic connection between the material in the unconscious mind and conscious mind to experience an “ah ha” or “eureka” moment.

As it relates to the two “emotional” types of creativity, but without being bound by theory, the portion of the brain largely responsible is the amygdala. Here, creativity is driven by one's emotional state. An example of deliberate emotional creativity would be an actor recalling a particularly devastating memory to portray a character experiencing something similar. Likewise, when a person is in a difficult, depressing, or dangerous situation and manifests a positive disposition through meditation or conjuring positive thoughts, they are exhibiting deliberate emotional creativity. Similarly, spontaneous emotional creativity occurs when emotion drives the creation of a masterpiece-like that of a novel, play, musical movement, or film. One cannot control the flow of information when experiencing this type of creativity, but must have the skills to translate/decode their thoughts into a tangible product. Thus, if one can mentally “hear” the perfect melody, but is unable to reproduce the chords, the “flash of genius,” so to speak, is lost.

As it relates embodiments of the disclosure, improvements in creativity include improvements to any of deliberate cognitive creativity, deliberate emotional creativity, spontaneous cognitive creativity, spontaneous emotional creativity, or combinations thereof. Objectively, this may be measured using “divergence tests,” psychometrics, the social-personality approach, and/or various questionnaires (Kaplunov, 2021). “Divergence tests” may ask a subject a set of questions employing imagination and open-mindedness, and consider the “uniqueness” of a response. Psychometrics may utilize questionnaires to measure people's skills, character traits, educational attainment, and knowledge to ascertain their attitudes, behaviors, and thinking (Kaplunov, 2021). The “Creative Achievement Questionnaire,” or (CAQ), may ask subjects to rate their achievements in the creative domains of visual arts, music, dance, architectural design, creative writing, humor, inventions, scientific discovery, theatre and film, and culinary arts (Kaplunov, 2021). The social-personality approach may measure creativity by measuring personality factors and categorizing them as types of creativity-non-limiting examples of which include risk-taking, aesthetic orientation and attitudes, interested in complexity, confidence, and independence of judgement (Kaplunov, 2021). Further, as will be known to those of skill, there are a host of questionnaires specializing in assessing creativity, three of which include the alternative use test, which challenges a participant to think of as many uses as possible for a “simple object,” a non-limiting example of which may include a paperclip, a brick, or a shoe (Creative Huddle, 2021); the remote association test, which consists of an exam having three common seemingly-unrelated “stimulus” words, wherein a participant is required to present a fourth, associated word—the score of the exam reflecting the amount of correct answers given by the participant (Backman and Tuckman, 1972); and the Wallach-Kogan test, which asks examinees to name as many items as possible in a given group, such as “objects with wheels,” etc. (Dabell, 2020). Other ways to objectively measure creativity include targeting specific types of creativity, and tailoring pre- and post-administration experiments, such as described in the Examples sections herein.

In embodiments, the entactogenic combinations can be said to foster creativity through internal and external means, the former by creating a low degree of psychological defensiveness, a lack of rigidity and permeability of boundaries in concepts, beliefs, perceptions, and hypotheses; tolerance for ambiguity where it exists, the ability to receive and integrate apparently conflicting information, creating sensitive awareness of feelings and openness to all phases of experience, evaluating judgment based primarily on one's own feelings, intuition, aesthetic sensibility, sense of satisfaction in self-expression, etc.; the ability to “toy” with ideas, colors, shapes, hypotheses; the ability to translate from one form to another, and the ability to think in terms of analogs and metaphors; the creation of an atmosphere of psychological safety, wherein the individual feels accepted as of unconditional worth and can be spontaneous without fear that the individual's actions or creations will be prematurely evaluated by rigid external standards; and a feeling of empathic understanding and psychological freedom, wherein the individual feels able to think, feel, or be whatever is discovered within themself (Harman et al., 1966).

In some embodiments, disclosed combinations are useful in enhancing, increasing, magnifying, amplifying, strengthening, boosting, or augmenting “creativity” or the creative process. In embodiments, “creativity” may refer to any of artistic creativity and professional creativity, which includes problem solving, generating new and inventive ideas, creating more efficient processes, etc.

In some embodiments, an improvement in mental health and functioning is a decrease in social awkwardness. As it relates to the invention, “social awkwardness” may refer to the feeling experienced when a person believes their desire for being accepted by others is threatened in a given situation, so they turn inward, increase self-monitoring, and attempt to behave in ways that will better their chances for acceptance (Clegg, 2012).

The entactogenic combinations are effective at treating social awkwardness by, for example, increasing the empathy of the subject. “Empathy” may refer to the ability to understand and share the feelings of others. The entactogenic combinations are also effective in treating other aspects of social awkwardness. Social awkwardness may stem from one's desire to be accepted, and fear that their actions in a given situation may jeopardize said acceptance. The entactogenic effects of disclosed combinations and compositions can in some embodiments allow a subject to possess the ability to feel more “present” and “comfortable” in a social setting, reducing one's fear of losing acceptance. In some embodiments, the entactogenic effects of disclosed combinations are measured according to known methods (Clegg, 2012).

In some embodiments, disclosed combinations are used to treat social awkwardness. In some embodiments, disclosed combinations are used to limit, diminish, reduce, attenuate, lessen, decrease, or weaken the effects of social awkwardness experienced by a subject.

The disclosed combinations and compositions and the entactogenic mindstates produced thereby are not only therapeutically useful to treat CNS disorders and conditions, but also for improvements in issues that may not result in a clinical diagnosis of a specific “disorder,” and may also be beneficially used for the betterment of the well and the “healthy,” such as those termed “healthy normals.” In embodiments, the disclosed combinations and compositions are used by the “healthy,” and for the betterment of the well.

In embodiments, the entactogenic compositions are used for the creation of emotional connection with others or with the spiritual, the creation of euphoric connection with others or with the spiritual, the creation of ecstatic communion with others or with the spiritual, to encouragement of respectfully working through differences between partners or groups, and the general improvement of partner or group interaction, bonding, and cohesion.

### N. Reduction of Adverse Effects Relative to MDMA and Other Entactogens

Central to certain improvements and advantages of the invention is the ability to produce an entactogenic mindstate using a disclosed combination or composition, and producing the therapeutic effects of, without the adverse effects associated with, currently known entactogens, such as 1-(1H-indol-3-yl)butan-2-amine (α-ethyltryptamine), 1-(1H-Indol-3-yl) propan-2-amine (α-methyltryptamine), 2,5-dimethoxy-4-bromo-phenethylamine (2C-B), 2-(Methylamino)-1-(3-methylphenyl)propan-1-one (3-methylmethcathinone), 5-(2-Amino-propyl)benzofuran (5-APB), 5-(2-methylaminopropyl) benzofuran (5-MAPB), 5-iodo-2,3-dihydro-1H-inden-2-amine (5-IAI), 6-(2-Aminopropyl)benzofuran (6-APB), 6-(2-aminopropyl)-2,3-dihydrobenzofuran, (6-APDB), 5-(2-Aminopropyl)-2,3-dihydro-1H-indene (5-APDI, IAP), Methylbenzodioxolylbutanamine (MBDB), 5-Methoxy-2-aminoindane (MEAT), 3,4-Methylenedioxyamphetamine (MDA), 5,6-Methylenedioxy-2-aminoindane (MDAI), 3,4-Methylenedioxy-N-ethyl amphetamine (MDEA), Methylenedioxy-hydroxyamphetamine (MDOH), (RS)-2-Methylamino-1-(4-methylphenyl)propan-1-one, (mephedrone) 3,4-Methylenedioxy-N-methylcathinone (methylone), 1-(7-Methoxy-1,3-benzodioxol-5-yl) propan-2-amine (MMDA), para-Methoxymethamphetamine (PMMA), 3,4-methylenedioxy-meth amphetamine (MDMA), and others (see, e.g., Oeri, 2021).

Generally, MDMA and these other entactogens produce no long-lasting or serious adverse events. However, they are known to cause transient adverse events that are mild to moderate in severity, including increased anxiety, cardiovascular effects such as increased blood pressure and heart rate, hyperthermia, hyperhidrosis, jaw tightness and bruxism, muscle tightness, unpleasant stimulation, reduced appetite, nausea, poor concentration, and impaired balance (see, e.g., Harris 2002, Lietchti 2001, Oehen 2013, Mas 1999, Mithoefer 2010, Rogers 2009). Accordingly, among the advantages of the invention are the ability of the therapeutic combinations disclosed herein to harness the therapeutic benefits of entactogens such as MDMA without their negative side effects. Mitigating one or more side effects and improving the safety profile of an entactogenic composition increases the value of the composition for therapeutic use, and broadens the population of patients who could benefit from treatment, including by providing the potential for increased access outside of supervised settings in the clinic.

Moreover, as mentioned, when MDMA is used as a probe in the context of the invention, when paired with a primer that is any of a CB₁ agent, 5-HT₂ agent, and 5-HT₇ agent, or a combination thereof, the dose of MDMA may be substantially lower than that of a standard dose required to subjectively perceive the entactogenic effects of MDMA. As such, such transient adverse effects are expected to be significantly reduced in both magnitude and incidence.

Measurements of adverse effects will be understood by those in the art; for example a subject may be asked to complete a self-report symptom questionnaire, such as the Subjective Drug Effects Questionnaire (SDEQ) or List of Complaints, taken at an appropriate time following treatment. The SDEQ is a 272-item self-report instrument measuring perceptual, mood, and somatic changes, which has also been used to measure the therapeutic and adverse effects of MDMA (Katz et al. 1968. J Abnorm Psychology 73:1-14; Harris et al. 2002. Psychopharmacol, 162(4), 396-405). The List of Complaints is a 66-item questionnaire that measures physical and general discomfort and may be used to assess entactogen-related complaints (e.g., Vizeli & Liechti. 2017. J Psychopharmacol, 31(5), 576-588).

In embodiments, prevalence of one or more adverse effects will be decreased by 5%, 10%, 20%, 30%, 40%, 50%, 60%, 70%, 80%, 90%, 95%, 97.5%, 99%, or 100%, up to and including 100%, relative to a comparator entactogen, such as MDMA.

In embodiments, among the advantages is the reduction or elimination of tolerance, or a reduction or elimination of the so-called “loss of magic,” wherein individuals are unable to obtain an entactogenic mindstate or obtain certain (and generally desired) entactogenic effects when redosing, i.e., taking an entactogen after a previous administration of an entactogen, including when redosing after a specified period of time since the last administration, such as at least one day, at least two days, at least three days, at least four days, at least five days, at least six days, at least one week, at least two weeks, at least three weeks, at least four weeks, at least six weeks, at least eight weeks, at least three months, at least four months, at least five months, at least six months, at least seven months, at least eight months, at least nine months, at least ten months, at least 11 months, at least 1 year, or over 1 year, including over 2 or 3 years.

In embodiments, the disclosed combinations and compositions demonstrate a reduction in one or more adverse effects relative to a prior art or comparative compound, combination, or composition (e.g., MDMA, or a combination of MDMA and another compound). In embodiments, the reduction may be at least 5%, at least 10%, at least 15%, at least 25%, at least 50%, at least 75%, at least 90%, at least 95%, or at least 99%, or entirely without the adverse effect. In embodiments, there may be a reduction of the adverse effect when measured using another standard such as known to one of skill for determining or quantifying the adverse effect, including tests and procedures that are in silico (e.g., by computer analysis or simulation and including by using AI, machine learning, or deep learning models), in vitro (e.g., biochemical assays, tissue culture, etc.), and in vivo (e.g., behavioral assessment; functional observational batteries; neurological neurochemical, neuroendocrine, or neuropathological measures; EEG; imaging; and the like).

The pharmacokinetics of MDMA, and its metabolites, can pose safety risks associated with toxicity (Kalant, 2001), such as neurotoxicity. Moreover, some MDMA metabolites, such as MDA, retain pharmacological activity and extend the duration of action, which can increase the likelihood of toxicity. The catechol moieties of MDMA and certain metabolites thereof, such as MDA, are postulated to be inherently reactive. Downstream effects of such reactivity include generation of reactive oxygen species, reactive nitrogen species, and other toxic byproducts (Carvalho, 2010). In some embodiments, disclosed compositions, combinations, and methods do not produce a neurotoxic effect. In some embodiments, disclosed compositions, combinations, and methods produce a reduced neurotoxic effect, as compared to MDMA or another entactogen. In some embodiments, the absence or reduction of a neurotoxic effect is determined by measuring one or more of: a) at least one toxic metabolite of MDMA or at least one toxic metabolite of another entactogen; b) oxidative stress and dopamine-based quinones; c) mitochondrial dysfunction; and d) activation of glial cells.

### O. Determination of an Entactogenic Mindstate

The determination of an entactogenic mindstate may be measured using one or more questionnaires or assessments, such as the Brief Fear of Negative Evaluation-revised (BFNE) (Carleton et al., 2006, Depression and Anxiety, 23(5), 297-303; Leary, 1983, Personality and Social Psychology bulletin, 9(3), 371-375), the Authenticity Inventory (Kernis & Goldman. 2006. Advances in experimental social psychology, 38, 283-357) as modified by Baggott et al (Journal of Psychopharmacology 2016, 30.4: 378-87), the Abnormal Mental States questionnaire, first published in 1998 by Adolf Dittrich, which includes three dimensions, including “Oceanic Boundlessness (OSE),” “Dread of Ego Dissolution (AIA),” and Visionary Restructuralization (VUS),” Positive and Negative Symptom Schedule-X (PANAS-X), Brief Fear of Negative Evaluation (BFNE), Visual Analog Scale Items (VAS), and Trustworthy Face Task (TFT), the Subjective Drug Effects Questionnaire (SDEQ), and such other questionnaires or assessments as will be known to those of skill (including, without limitation, the Altered States of Consciousness Rating Scale, the Adjective Mood Rating Scale, the List of Complaints, the State-Trait Anxiety Inventory, the EWL Mood Questionnaire, the Visual Activity Scale, the Drug Effects Questionnaire, the Interpersonal Attraction Questionnaire, the Perceived Responsiveness Questionnaire, the Social Interaction Questionnaire, a Video Recording of Social Interactions, the Facial Emotion Recognition Task, the Multifaceted Empathy Test, a Movie for the Assessment of Social Cognition, Social Value Orientation, a Moral Judgment Test, the Intra/Extradimensional Attentional Set Shifting Task, a Rapid Visual (Information) Processing Task, the Severity of Symptoms Scale for PTSD, the Beck Depression Inventory, the Hamilton Rating Scale, the Modified Fear Scale, the Maladjustment Scale, the Rosenberg Self-Esteem Scale, and the Two-Choice Prediction Task). In some embodiments, including certain preferred embodiments, at least two or more questionnaires or assessments will be used in conjunction, in parallel, or otherwise together, such as two or more, three or more, four or more, five or more, six or more, seven or more, eight or more, nine or more, ten or more, or greater than ten questionnaires or assessments.

In embodiments, the determination of an entactogenic mindstate is completed by using machine learning (ML), natural language processing (NLP), and/or other analysis of text such as subjective reporting (e.g., trip reports, free form journaling, or free speech recording, including both speech intentionally recorded for such purposes and speech recorded during a drug administration or non-drug administration session, such as in EAP, or passively recorded such as during another activity). In embodiments, an individual may speak directly about their experience into a microphone, the microphone connected to a machine capable of recording, optionally transcribing, and analyzing the spoken word, and identifying “key words” or “key phrases” which are programmed or recognized as being indicative of (“markers” of) an entactogenic mindstate. In embodiments, an individual's freeform or non-directed speech is recorded and preserved for later use. In embodiments, an individual's speech is not recorded.

In embodiments, a subject's words are obtained from published writings or obtained as recorded audio, optionally, transcribed, and subsequently processed, e.g., using ML/NLP, and used to create markers of an entactogenic mindstate, or compared to previously created markers. For example, audio of spoken word from Bessel Van Der Kolk, author of “The Body Keeps the Score,” recorded on “The Ezra Klein Show,” Aug. 24, 2021, as transcribed, is processed to generate or contribute to markers of an entactogenic mindstate; a transcription reads: “And our current research is very much also in psychedelic agents, which turn out to have a very dramatically positive effect on exactly the courses of defectiveness, self-loathing, internal confusion. And our latest data really show that MDMA particularly can really help people to get a much deeper sense of who they are and a deep sense of compassion for themselves . . . . I think psychedelics are a true revolution. And it's partially a revolution because we don't know how they work. There's all these explanations: oh, the serotonin system or the default mode network. But what has happened in the world we live in is that people pay varied attention to what happens in the mind. And what we see in our MDMA research is that MDMA seems to really trigger a capacity to look at yourself in a more compassionate way, in the same way that MDMA is being used at parties. And when we do our work, very painful, painful, old things tend to come up. But people are able to go there and not get overwhelmed by shame, or overwhelmed by the horror. And they're able to go there and say, yeah, this is what happened to me. Oh my God, that was awful. And actually . . . what you see is a dramatic increase in self compassion. People say, I now can really see what I went through, and how awful it was, and how I have survived it. And what's fascinating to me is that it gives people a sense of perspective, of who they are, and their sense of self gets very much enhanced. This is who I am, this is what I went through, and this is what I've learned from experience. It's really very dramatic, and I think it's the sort of thing that may happen with very long term psychotherapy, but I think very profound things can happen in a very short period of time.”

In embodiments, a subject's words are transcribed into a freeform or structured “trip journal,” either by the subject, someone recording the subject and later transcribing the audio, or an automated “text-to-speech” service, and further analyzed through machine learning to identify patterns, phrases, or words in the transcribed speech indicative of an entactogenic response (i.e., markers of an entactogenic mindstate).

In embodiments, the trip journals of individuals are collected and analyzed by natural language processing (NLP), for example using ML, wherein each individual lists I₁ agents taken by the individual, together with other (i.e., 5-HT₂, 5-HT₇, and/or CB₁) agents taken in combination, including the dose administered, the time of administration, and the route of administration; provided together with a detailed report of the subjective effects, the time of the onset of effects, and the duration of the effects.

### P. Equivalent or Greater Entactogenic Mindstates Versus Known Entactogens

In some embodiments, a disclosed combination or composition will produce an entactogenic mindstate that is similar to the entactogenic mindstate of a known entactogen (a “comparator” entactogen). In embodiments, an entactogenic combination will therefore produce an entactogenic mindstate that is similar to, for example, any one or more of 1-(1H-indol-3-yl)butan-2-amine (α-ethyltryptamine), 1-(1H-Indol-3-yl)propan-2-amine (a-methyltryptamine), 2,5-dimethoxy-4-bromo-phenethylamine (2C-B), 2-(Methylamino)-1-(3-methylphenyl)propan-1-one (3-methyl-methcathinone), 5-(2-Aminopropyl) benzofuran (5-APB), 5-(2-methylaminopropyl) benzofuran (5-MAPB), 5-iodo-2,3-dihydro-1H-inden-2-amine (5-IAI), 6-(2-Aminopropyl)benzofuran (6-APB), 6-(2-aminopropyl)-2,3-dihydrobenzofuran, (6-APDB), 5-(2-Aminopropyl)-2,3-dihydro-1H-indene (5-APDI, IAP), Methylbenzodioxolylbutanamine (MBDB), 5-Methoxy-2-aminoindane (MEAT), 3,4-Methylenedioxyamphetamine (MDA), 5,6-Methylenedioxy-2-aminoindane (MDAI), 3,4-Methylenedioxy-N-ethyl amphetamine (MDEA), Methylene-dioxyhydroxy-amphetamine (MDOH), (RS)-2-Methylamino-1-(4-methylphenyl)propan-1-one, (mephedrone) 3,4-Methylenedioxy-N-methylcathinone (methylone), 1-(7-Methoxy-1,3-benzodioxol-5-yl) propan-2-amine (MMDA), para-Methoxymethamphetamine (PMMA), 3,4-methylenedioxymethamphetamine (MDMA), and others (see, e.g., Oeri, 2021).

In embodiments, a disclosed combination or composition will produce an entactogenic mindstate that is substantially similar to the entactogenic mindstate of a known entactogen. In embodiments, a disclosed combination or composition will produce an entactogenic mindstate that is substantially similar to the entactogenic mindstate of any one or more of the above entactogens. In embodiments, a disclosed combination or composition will produce an entactogenic mindstate that is substantially similar to the entactogenic mindstate of MDMA. An entactogenic mindstate that is “similar” or “substantially similar” to that of an entactogen may be determined directly or indirectly, e.g., based on drug discrimination assessments as appreciated by those in the art, or based on comparisons of scores of entactogenic effects, including within or between subjects. In embodiments, “similar” will refer to the results of a comparison that are within 0.01, 0.1, 0.5, 1, 1.5, 2, or 2.5 percent of each other. The standard deviation as a percent of the average value across repeated experiments can be less than or up to 0.01, 0.1, 0.5, 1, 1.5, 2, or 2.5 percent for similar results. In embodiments, “substantially similar” will refer to the results of a comparison that are equal to 2.5 percent of each other, or within 3, 3.5, 4, 4.5, or 5 percent of each other. The standard deviation as a percent of the average value across repeated experiments can be greater than, more than, or about 2.5, 3, 3.5, 4, 4.5, or 5 percent for substantially similar results.

In other embodiments, a disclosed combination, composition, or method will produce an entactogenic mindstate that is greater than the entactogenic mindstate of a known entactogen (a “comparator”), where “greater” may be “expanded” or “broader” (i.e., a larger number of entactogenic effects) and/or “enhanced” or “deeper” (i.e., a higher score on one or more entactogenic effects). In embodiments, “greater” will refer to the results of a comparison where the disclosed combination or composition has a score that is more than 5, 6, 7, 8, 9, 10, 11, 12, 13, 14, 15, 16, 17, 18, 19, or 20 percent than the comparator. In other embodiments, “greater” will refer to the results of a comparison that are more than 25, 30, 35, 40, 45, 50, 55, 60, 65, 70, 75, 80, 85, 90, 95, or 100 percent than the comparator.

In embodiments, the entactogenic mindstate can be evaluated based on subjective reports. Non-limiting examples of subjective reports include non-verbal, verbal, or written (e.g., hand-written, or typed using a physical or digital keyboard) descriptions of effects. In embodiments, non-verbal, verbal, or written descriptions of effects are reported or recorded on a scale or subject to characterization to determine the magnitude of the entactogenic mindstate. In embodiments, the entactogenic mindstate can be evaluated based on observer reports. Non-limiting examples of observer reports include verbal or written (e.g., hand-written, or typed using a physical or digital keyboard) descriptions of observed effects. In embodiments, verbal or written descriptions of effects are reported or recorded on a scale or subject to characterization to determine the magnitude of the entactogenic mindstate.

In embodiments, the magnitude of the entactogenic mindstate is correlated with a reduction of one more symptoms of a condition. In embodiments, the entactogenic mindstate causes a reduction in one or more symptoms of a condition. In embodiments, the one or more symptoms is associated with a CNS disorder or condition, as described herein. In embodiments, the reduction in one or more symptoms can be evaluated or measured according to any method known to one of skill in the art. In embodiments, the reduction in one or more symptoms can be evaluated or measured via “objective measurements,” as disclosed herein. In embodiments, the reduction in one or more symptoms can be measured, tracked, or compared using subject or observer reported measures. In embodiments, the subject or observer reported measures are specific to a particular condition. Non-limiting examples of such measures include the Columbia-Suicide Severity Rating Scale (C-SSRS), Hospital Anxiety and Depression Scale (HADS), Generalized Anxiety Disorder Scale-7 (GAD-7), Posttraumatic Diagnostic Scale (PDS), and a Clinician Administered PTSD Scale (CAPS), for example, CAPS-1 and CAPS-5.

In embodiments, the magnitude of the entactogenic mindstate can be evaluated by measuring, tracking, or comparing global functioning. Non-limiting examples of global functioning include the quality of functioning in day-to-day activities and in relationships with the self, family, friends, and colleagues. In embodiments, the subject- or observer-reported measures capture global functioning. In embodiments, the subject- or observer-reported measures are not specific to a particular condition. In embodiments, the subject- or observer-reported measures capture the extent or severity of symptoms, which are not necessarily tied to a specific condition. Non-limiting examples of such measures include the Clinical Global Impressions-Severity (CGI-S) scale and the Subjective Units of Disturbance scale (Busner, 2007; Mithoefer, 2015; Robertson). In embodiments, a reduction in symptoms may result in an increase in well-being and/or feelings of improvement. One example of such measures is the CGI-I (Improvement) scale (Busner, 2007).

In embodiments, the magnitude of the entactogenic mindstate can be determined by evaluating a response to certain stimuli. Such a response can be determined using subjective reports, observer reports, or at least one of the “objective measurements” disclosed herein. In embodiments, the objective measurements further comprises determining levels of one or more neurotransmitters. Non-limiting examples of such compounds include serotonin, dopamine, norepinephrine, oxytocin, and cortisol.

In some embodiments, the magnitude, qualities, and/or characteristics of the entactogenic mindstate are determined by measuring the levels of one or more of serotonin, dopamine, norepinephrine, oxytocin, and cortisol. In some embodiments, the entactogenic mindstate is determined by measuring serotonin levels. In some embodiments, the entactogenic mindstate is determined by measuring dopamine levels. In some embodiments, the entactogenic mindstate is determined by measuring norepinephrine levels. In some embodiments, the entactogenic mindstate is determined by measuring oxytocin levels. In some embodiments, the entactogenic mindstate is determined by measuring cortisol levels. In some embodiments, the neurotransmitter levels are measured in vivo. In some embodiments, the neurotransmitter levels are measured ex vivo (e.g., in plasma).

In some embodiments wherein a disclosed combination, composition, or method produces an entactogenic mindstate that is “similar” to a comparator (e.g., MDMA, or another known entactogen), similarity of mindstates is determined by comparison of neurotransmitter levels associated with the entactogenic mindstate produced by a disclosed combination, composition, or method, as compared to the comparator mindstate. In some embodiments, “similar” refers to the results of such comparison, when the levels of a measured neurotransmitter are within 50%, 40%, 30%, 20%, 10%, 5%, or less than 1% of each other. In some embodiments, an entactogenic mindstate is “similar” to a comparator mindstate when the measured serotonin level associated with the entactogenic mindstate is within 50%, 40%, 30%, 20%, 10%, 5%, or less than 1% of the measured serotonin level associated with the comparator mindstate. In some embodiments, an entactogenic mindstate is “similar” to a comparator mindstate when the measured dopamine level associated with the entactogenic mindstate is within 50%, 40%, 30%, 20%, 10%, 5%, or less than 1% of the measured dopamine level associated with the comparator mindstate. In some embodiments, an entactogenic mindstate is “similar” to a comparator mindstate when the measured norepinephrine level associated with the entactogenic mindstate is within 50%, 40%, 30%, 20%, 10%, 5%, or less than 1% of the measured norepinephrine level associated with the comparator mindstate. In some embodiments, an entactogenic mindstate is “similar” to a comparator mindstate when the measured oxytocin level associated with the entactogenic mindstate is within 50%, 40%, 30%, 20%, 10%, 5%, or less than 1% of the measured oxytocin level associated with the comparator mindstate. In some embodiments, an entactogenic mindstate is “similar” to a comparator mindstate when the measured cortisol level associated with the entactogenic mindstate is within 50%, 40%, 30%, 20%, 10%, 5%, or less than 1% of the measured cortisol level associated with the comparator mindstate.

In some embodiments wherein a disclosed combination, composition, or method produces an entactogenic mindstate that is “greater” than a comparator (e.g., MDMA, or another known entactogen), or “expanded” relative to a comparator, similarity of mindstates is determined by comparison of neurotransmitter levels associated with the entactogenic mindstate produced by a disclosed combination, composition, or method, as compared to the comparator mindstate. In some embodiments, “greater” or “expanded” refers to the results of such comparison, when the levels of a measured neurotransmitter are 1.1-fold, 1.2-fold, 1.25-fold, 1.5-fold, 1.7-fold, 2-fold, 5-fold, 10-fold, 20-fold, or 50-fold higher in the entactogenic mindstate relative to the comparator mindstate. In some embodiments, an entactogenic mindstate is “greater” or “expanded” to a comparator mindstate when the measured serotonin level associated with the entactogenic mindstate is 1.1-fold, 1.2-fold, 1.25-fold, 1.5-fold, 1.7-fold, 2-fold, 5-fold, 10-fold, 20-fold, or 50-fold higher in the entactogenic mindstate relative to the comparator mindstate. In some embodiments, an entactogenic mindstate is “greater” or “expanded” to a comparator mindstate when the measured dopamine level associated with the entactogenic mindstate is 1.1-fold, 1.2-fold, 1.25-fold, 1.5-fold, 1.7-fold, 2-fold, 5-fold, 10-fold, 20-fold, or 50-fold higher in the entactogenic mindstate relative to the comparator mindstate. In some embodiments, an entactogenic mindstate is “greater” or “expanded” to a comparator mindstate when the measured norepinephrine level associated with the entactogenic mindstate is 1.1-fold, 1.2-fold, 1.25-fold, 1.5-fold, 1.7-fold, 2-fold, 5-fold, 10-fold, 20-fold, or 50-fold higher in the entactogenic mindstate relative to the comparator mindstate. In some embodiments, an entactogenic mindstate is “greater” or “expanded” to a comparator mindstate when the measured oxytocin level associated with the entactogenic mindstate is 1.1-fold, 1.2-fold, 1.25-fold, 1.5-fold, 1.7-fold, 2-fold, 5-fold, 10-fold, 20-fold, or 50-fold higher in the entactogenic mindstate relative to the comparator mindstate. In some embodiments, an entactogenic mindstate is “greater” or “expanded” to a comparator mindstate when the measured cortisol level associated with the entactogenic mindstate is 1.1-fold, 1.2-fold, 1.25-fold, 1.5-fold, 1.7-fold, 2-fold, 5-fold, 10-fold, 20-fold, or 50-fold higher in the entactogenic mindstate relative to the comparator mindstate.

In one illustrative study, brain imaging showed attenuated responses to angry facial expressions and enhanced responses to happy expressions in subjects who were administered MDMA (Bedi, 2009). Additionally, subjects reported increases in sociability following administration of MDMA. In another illustrative study, MDMA was administered to subjects who were then asked to read social cues from the Reading the Mind in the Eyes Test (Hysek, 2012). Similar to the Bedi study, it was determined that MDMA enhanced mental state decoding of positive stimuli, such as friendliness, and impaired the accuracy of mind reading for negative stimuli. The results of such studies show aspects of the entactogenic mindstate related to sociability, diminished responses to threatening stimuli, and enhanced responses to pleasant or rewarding social signals. Such studies demonstrate that production of an entactogenic mindstate, as achieved by administering MDMA, may be measured or characterized with a variety of methods.

In embodiments, the magnitude, qualities, and/or characteristics of the entactogenic mindstate are determined by brain imaging experiments conducted using methods known in the art. In embodiments wherein a disclosed combination, composition, or method produces an entactogenic mindstate that is “similar,” “greater than,” or “expanded” relative to a comparator (e.g., MDMA, or another known entactogen), similarity of mindstates is determined by comparison of characteristics measurable by brain imaging.

In another method, in embodiments, an entactogenic mindstate will be measured or characterized using the using the Brief Fear of Negative Evaluation-revised (BFNE) (Carleton et al., 2006) or the Authenticity Inventory (Kemis & Goldman, 2006; as modified by Baggott et al., 2016, which consists of a number of items which are rated on a scale from 1 to 5, or any one or more of such other questionnaires and assessments as are described herein or known in the art.

In embodiments, the combinations described herein elicit an entactogenic mindstate, like that produced by MDMA. In embodiments, the combinations described herein are expected to produce similar effects to MDMA. In embodiments, the combinations described herein are expected to produce enhanced effects in comparison to MDMA. In embodiments, the provided combinations do not cause undesirable physiological effects, such as those characterized by MDMA administration. In embodiments, subject-reported and/or observed effects following administration of a combination provided herein are expected to be similar to those following administration of MDMA. In embodiments, subject-reported and/or observed effects following administration of a combination provided herein are expected to be enhanced relative to those following administration of MDMA.

### Q. Examples

That the disclosed combinations and compositions are useful to treat the conditions and disorders discussed herein may be shown in part by the following Examples. It will be readily appreciated that these Examples are merely exemplary and are intended to be illustrative and not to be limiting.

a. Studies and Treatment Examples

### Example 1: Deliberate Cognitive Creativity Improvement Study

To measure the efficacy of a disclosed entactogenic combination in improving deliberate cognitive creativity, an individual is given a task within the realm of their own knowledge, e.g., a problem dealing with their work or a hobby, and then given a set amount of time to solve the problem, with their progress measured. The individual is then administered an effective amount of a disclosed combination or composition, and made to repeat the experiment with a different, but similar problem. The results will indicate that the individual either solved the problem more rapidly after administration of an effective amount of the disclosed combination or composition, or made more progress in solving the problem while experiencing the effects of the disclosed combination or composition.

### Example 2: Alternative Deliberate Cognitive Creativity Improvement Study

To measure the efficacy of a disclosed entactogenic combination in improving deliberate cognitive creativity, an individual is given a task within the realm of their own knowledge, e.g., a problem dealing with their work or a hobby, and then is observed while they attempt to solve the problem. Observation includes assessing the individual's levels of stress (which may be indicated as known to those of skill, but may be noted by visually observing the individual, or obtaining objective measurements that may indicate stress, such as heart rate, breathing rate, blood pressure, etc.) Observation will also include logging the amount of time necessary to solve the problem. The individual will then be administered an effective amount of a disclosed combination or composition, and made to repeat the experiment with a different, but similar problem (i.e., similar in level of difficulty and scope). The results will indicate that the individual solved the problem more rapidly after administration of an effective amount of a disclosed entactogenic combination, and will indicate that the individual showed less indications of stress, including but not limited to visually observable signs, and those obtained from objective measurements, as disclosed herein.

### Example 3: Spontaneous Cognitive Creativity Improvement Study

To measure the efficacy of a disclosed entactogenic combination in improving spontaneous cognitive creativity, an individual will be given a first problem to solve, and then given a second task that requires their full attention to complete. The individual will then be given a set amount of time to complete the second task, and the individual's progress on the first problem will be assessed at the conclusion of the given set amount of time. Then, the individual is administered an effective amount of a disclosed combination or composition, and made to repeat the experiment with a different (but similar in scope and difficulty) first problem and second task. Results will indicate that the individual more effectively solved the first problem and second task—i.e., the individual either solved each problem more rapidly after administration of an effective amount of a disclosed entactogenic combination, or made more progress in solving the problem while experiencing the effects of the disclosed combination or composition.

### Example 4: Deliberate Emotional Creativity Improvement Study

To measure the efficacy of a disclosed entactogenic combination in improving spontaneous cognitive creativity, an individual will be given a scene to act out, or a disposition to conjure. Effects of disclosed combinations will be apparent in both the subjective experience of the subject, and the objective analysis of those administering the test.

In this exemplary study, an individual will be given three separate emotions to portray—e.g., joy, sadness, anger, shock, devastation, terror, and happiness—and then, throughout the administration of the test, the individual will be assessed on a scale of 1-10, with 1 being the least expressive and 10 being the most expressive. Through capture of the testing session via video and audio, those administering the test will objectively grade the performance of each subject. Specifically, a proctor of the test will give the individual a spoken line, and then an emotion to display, such as “anger 7,” “joy 5,” “terror 9,” etc., with one of skill appreciating that any of the emotions stated above may be given to the subject, and any number, between 1-10, may accompany such emotions. Once the first phase of the experiment is complete, the subject is administered an effective amount of the disclosed combination or composition, and the test is repeated. The proctors of the test will then review the video footage and rate the performance of each participant with and without the entactogenic combination. After the test proctors review the footage, it will be discovered that the disclosed combination or composition proved effective in increasing the ease with which the individual could portray the emotions and their associated intensities.

### Example 5: Spontaneous Emotional Creativity Improvement Study

To measure the efficacy of a disclosed entactogenic combination in improving spontaneous cognitive creativity, an individual is given a task to complete that is tailored to the individual and their respective talents, for example, creative writing, technical writing, poetry writing, creating a musical composition, writing lyrics for a given tune, writing dialogue for a given scene in a play or film, creating works of visual art, including paintings, drawings, wood working, etc. The subject is then given a set amount of time to complete as much of the task as possible within that time frame. Once completed, the individual is then administered an effective amount of the disclosed combination or composition, and repeats a similar task with a separate prompt, e.g., a music producer is told to compose a similar but different song, a poet is told to write a similar but different poem, etc., and—as in the first experiment—the individual is given a set amount of time to complete as much of the task as possible. The results are then compared to the first experiment. At the conclusion of the experiment, it will be determined that administration of the disclosed entactogenic combinations is effective in improving spontaneous cognitive creativity in an individual, reflective in the ease at which the individual is able to complete the task assigned to them.

### Example 6: Alternative Spontaneous Emotional Creativity Improvement Study

To measure the efficacy of a disclosed entactogenic combination in improving spontaneous cognitive creativity, an individual is given a task to complete that is tailored to the individual and their respective talents, for example, creative writing, technical writing, poetry writing, creating a musical composition, writing lyrics for a given tune, writing dialogue for a given scene in a play or film, creating works of visual art, including paintings, drawings, wood working, etc. The subject is then allowed to work on the task until they feel the task is complete. Once completed, the individual is then administered an effective amount of the disclosed combination or composition, and repeats a similar task with a separate prompt, e.g., a music producer is told to compose a similar but different song, a poet is told to write a similar but different poem, etc., and—as in the first experiment—the individual is allowed to work on the task until they feel the task is complete. The results are then compared to the first experiment. At the conclusion of the experiment, it will be determined that administration of the disclosed entactogenic combinations is effective in improving spontaneous cognitive creativity in an individual, reflective in the ease at which the individual is able to complete the task assigned to them.

For all of the above examples, as would be readily appreciated by one of skill, the way in which time is measured for both sets of experiments is consistent across each experiment. Thus, if an individual was given as much time as they desired to complete the task in the first experiment, they will also be given as much time as they desire to complete the task in the second experiment. Likewise, if they are given a set amount of time and their progress is measured at the end of such duration of time in the first experiment, they should be given the same set amount of time, with their progress being measured at the end of such duration in the second experiment—so that the results may be compared. As it relates to the time allotted, as would be understood by one of skill, the time given will depend on the task given, and the type of creativity being measured.

Additionally, the above-referenced experiments objectively measuring an increase in creativity may be completed as a “zero blind” study, wherein the participants are aware they are taking an effective amount of the disclosed combination or composition, or through use of a placebo, in either a single-blind, or double-blind setting, the former being where the proctors of the test are aware of whether the participant is being administered a placebo or an effective amount of the disclosed combination or composition, and latter being where neither the subject or the proctor of the experiment are aware which administration is the placebo, and which is an effective amount of the disclosed combination or composition. Likewise, the order of administration may be altered. Meaning, a person may be administered the placebo for a first experiment, and an effective amount of the disclosed combination or composition for a second experiment, or a person may first be administered an effective amount of the entactogenic combination, followed by a separate test wherein the placebo is administered.

### Example 7: Social Awkwardness Reduction Study

To determine whether a disclosed entactogenic combination is efficacious in reducing social awkwardness, a study will be completed wherein ten individuals who have self-reported cases, or are diagnosed with a form of social awkwardness are invited into a room and told to sit in a chair, wherein the chairs form a circle that allows each individual to see all other individuals in the room. The individuals will then be told to engage in small talk and find out at least one surprising fact about the person sitting directly across from them in the circle. After this is completed, the individuals will be told to share the surprising fact they found out about the person sitting across from them.

Then, the individuals will be asked a series of polarizing questions, such as those revolving around religion, politics, social norms, etc., and will be asked to give their opinion about the topics to the members of the group present. Each individual will be asked to respond to at least one of the questions posed, wherein a total of 10 questions will be asked. At the conclusion of the question and answer session, the individuals will be asked to rank, on a scale of 1-10, the awkwardness felt during each portion of the experiment. Meaning, the awkwardness felt while engaging in small talk, when asking the individual across the circle from them a surprising fact about themselves, and the awkwardness felt when answering or listening to answers given during the question and answer session.

The study will then be repeated with a separate set of individuals diagnosed with, or having self-reported cases of social awkwardness, but prior to coming into the room, the individuals are administered an effective dose of the disclosed entactogenic combinations. As with the previous group of individuals, these individuals will also be told to document, on a scale of 1-10, subjective rankings of awkwardness felt during the different stages of the experiment. At the conclusion of the study, it will be discovered that the group of individuals having been administered an effective dose of a disclosed entactogenic combination perceived less social awkwardness than the group of individuals who were not administered the disclosed combination or composition.

### Example 8: Use in an Exemplary Psychedelic-Assisted Therapy Treatment

The disclosed compounds are administered in a therapist-supervised dosing session in a therapeutically effective amount to a patient with a CNS disorder, including a mental health condition, such as but not limited to post-traumatic stress disorder (PTSD), adjustment disorder, affective disorder, depression, atypical depression, postpartum depression, catatonic depression, a depressive disorder due to a medical condition, premenstrual dysphoric disorder, seasonal affective disorder, dysthymia, anxiety, phobia disorders, binge disorders, body dysmorphic disorder, alcohol or drug abuse or dependence disorders, substance-related disorders, substance-induced mood disorder, a mood disorder related to another health condition, disruptive behavior disorders, eating disorders, impulse control disorders, obsessive compulsive disorder (OCD), attention deficit hyperactivity disorder (ADHD), personality disorders, attachment disorders, and dissociative disorders; and neurodegenerative conditions, such as but not limited to Alzheimer's disease, ataxia, Huntington's disease, Parkinson's disease, motor neuron disease, multiple system atrophy, progressive supranuclear palsy, migraines, cluster headaches, short-lasting unilateral neuralgiform headaches, fibromyalgia, traumatic brain injury, and mild traumatic brain injury (mTBI).

Prior to the administration, the patient is evaluated to determine whether the therapist-supervised dosing session is appropriate for the patient. The evaluation includes completing questionnaires, obtaining objective health measurements from the patient, (herein, “objective measurements,”) including but not limited to weight, body temperature, heart rate (HR), respiratory rate, blood oxygenation, blood pressure (BP) and its variables, including, but not limited to: systolic (SBP), diastolic (DBP), mean arterial (MAP), and pulse (PP); continuous non-invasive beat-by-beat blood pressure (CNIBP); measurements from an electrocardiogram (ECG), including RR interval or its variability, QT interval or its variability, heart rate variability (HRV) (or measured by devices other than an ECG); hemodynamic response (HR), and levels of glucose, cortisol, serotonin, dopamine, cholesterol; electroencephalography (EEG) measures such as quantitative EEG (qEEG); electrocochleogram (ECochG), electromyography (EMG), electrooculography (EOG), magnetoencephalography (MEG); electrocorticography (ECoG); magnetic resonance imaging (MRI); functional MRI (fMRI); computed tomography (CT); positron emission tomography (PET); nuclear magnetic resonance (NMR) spectroscopy or magnetic resonance spectroscopy (MSR); single-photon emission computed tomography (SPECT); near infrared spectroscopy (NIRS); event-related optical signal (EROS); computed axial tomography; diffuse optical imaging (DOI); cranial ultrasound; or functional ultrasound imaging (fUS) (together, “EEG measures”); brain derived neurotrophic factor (BDNF); genetic markers including relating to CYP enzymes or drug metabolism; genetic variation in mGluR5 or FKBP5; and assessing the patient for “biomarkers” that may indicate a patient will have a sub-optimal response to psychedelic treatment, including those that may indicate poor metabolism of a psychedelic drug, such as but not limited to the CYP2D6 and CYP2B6 genes; those that may impact the response to serotonin, such as but not limited to the HTR2A gene; genes that may pose a mental health risk, such as but not limited to the C4A, NRG1, and DISC1 genes; the monoamine transporter genes, as discussed above; and physiological fluctuations that may indicate a patient will not perceive the full psychedelic experience, including but not limited to having a higher diversity of executive network nodes (i.e., less efficient network segregation), and having a lesser rostral anterior cingulate (rACC) thickness; and numerous more, as will be readily appreciated by those of skill. If the objective measurements obtained from the patient are within established safety standards known to those of skill, the patient will proceed with the dosing session.

Dosing is performed in a quiet clinical setting with the patient resting comfortably in an inclined, unrestrained position, and is supervised by at least one, but preferably two or more therapists, as part of a therapist team (dyad). The patient's eyes are covered, and music played, to create a calming atmosphere. A therapeutically effective dose of the disclosed entactogenic combinations is then administered. While under the effects of the same, the subject is encouraged to remain in a resting position, and to focus inwardly. Once the effects of the disclosed entactogenic combinations have subjectively or objectively ceased or are sufficiently reduced (i.e., after a duration of time wherein the therapy team is certain the blood concentration of the compounds described herein is such that all physiological effects will have ceased, or the physiological effects will be such as to render psychotherapy optimal), or the patient confirms subjective effects have ceased, the patient is permitted to stand and move around, or is engaged by the therapist in conversation to the extent desired by the patient, and in accordance with the treatment manuals discussed herein and known in the art for MDMA-assisted psychotherapy, such as for MDMA (see, e.g., Mithoefer, 2017; Mitchell, 2021). After about an hour of such free movement and further acclimatization, the patient is discharged. The following day (i.e., roughly 24 hours after the initial dosing session) the patient meets again with the therapy team and undergoes a psychotherapy session that recounts the experience.

### Example 9: Use as a Medication

The disclosed pharmaceutical compositions are prescribed in a therapeutically effective amount to a patient with a CNS disorder, such as a mental health condition, such as post-traumatic stress disorder (PTSD), adjustment disorder, affective disorder, depression, atypical depression, postpartum depression, catatonic depression, a depressive disorder due to a medical condition, premenstrual dysphoric disorder, seasonal affective disorder, dysthymia, anxiety, phobia disorders, binge disorders, body dysmorphic disorder, alcohol or drug abuse or dependence disorders, substance-related disorders, substance-induced mood disorder, a mood disorder related to another health condition, disruptive behavior disorders, eating disorders, impulse control disorders, obsessive compulsive disorder (OCD), attention deficit hyperactivity disorder (ADHD), personality disorders, attachment disorders, and dissociative disorders; and neurodegenerative conditions, such as but not limited to Alzheimer's disease, ataxia, Huntington's disease, Parkinson's disease, motor neuron disease, multiple system atrophy, progressive supranuclear palsy, migraines, cluster headaches, short-lasting unilateral neuralgiform headaches, fibromyalgia, traumatic brain injury, and mild traumatic brain injury (mTBI).

Prior to prescribing the medication, the patient is evaluated to determine whether a prescription is appropriate for the patient. The evaluation includes completing questionnaires, obtaining objective health measurements from the patient, including but not limited to weight, body temperature, heart rate, respiratory rate, blood oxygenation, BP and its variables, including, but not limited to: SBP, SBP, MAP, and PP; CNIBP; ECG measurements, including RR interval or its variability, QT interval or its variability, HRV (including measured by devices other than an ECG); hemodynamic response, and levels of glucose, cortisol, serotonin, dopamine, cholesterol; EEG measures; BDNF; genetic markers including relating to CYP enzymes or drug metabolism; genetic variation in mGluR5 or FKBP5. If the objective measurements obtained from the patient (if such measurements are taken) are within established safety standards known to those of skill, the patient is prescribed the medication containing the disclosed entactogenic combinations, wherein the prescribed dose is appropriate for the age and weight of the patient, taking into consideration other factors known to those of skill, including but not limited to comorbidities, current medications (if any), and metabolic variations among patient populations.

### Example 10: Self-Administration of the Disclosed Pharmaceutical Compositions

A patient administers an entactogenic combination disclosed herein to themselves. To do so, the patient obtains the pharmaceutical composition from a doctor, pharmacist, nurse practitioner, psychiatrist, or other such health professional capable of prescribing medication, and then self-administers the medication in a location of their choice, preferably is a calming, intimate, safe non-clinical setting; or a naturalistic setting meeting similar criteria, such as the patient's house, or any other location wherein the patient feels comfortable. After the patient self-administers a disclosed entactogenic combination, they remain in a comfortable area, and focus inwardly. The patient then records their thoughts and feelings in a journal, so that they are able to discuss the thoughts and feelings with a health professional at a later date.

### Example 11: Study Comparing the Entactogenic Combinations to a Known Entactogen

A study is to determine the efficacy of the entactogenic combinations as compared to MDMA (or to another known entactogen) taken by itself, wherein “efficacy” in this Example refers to the effectiveness of treating at least one CNS disorder, wherein CNS disorder broadly includes mental health conditions, such as but not limited to post-traumatic stress disorder (PTSD), adjustment disorder, affective disorder, depression, atypical depression, postpartum depression, catatonic depression, a depressive disorder due to a medical condition, premenstrual dysphoric disorder, seasonal affective disorder, dysthymia, anxiety, phobia disorders, binge disorders, body dysmorphic disorder, alcohol or drug abuse or dependence disorders, substance-related disorders, substance-induced mood disorder, a mood disorder related to another health condition, disruptive behavior disorders, eating disorders, impulse control disorders, obsessive compulsive disorder (OCD), attention deficit hyperactivity disorder (ADHD), personality disorders, attachment disorders, and dissociative disorders; and neurodegenerative conditions, such as but not limited to Alzheimer's disease, ataxia, Huntington's disease, Parkinson's disease, motor neuron disease, multiple system atrophy, progressive supranuclear palsy, migraines, cluster headaches, short-lasting unilateral neuralgiform headaches, fibromyalgia, traumatic brain injury, and mild traumatic brain injury (mTBI).

In this exemplary study, 30 participants (15 male, 15 female), diagnosed with at least one CNS disorder, as disclosed supra, are each administered either a disclosed combination or composition (10 people, 5 male 5 female), MDMA (or another known entactogen, such as MDA, MDEA, MDOH, MBDB, 5-APB, 5-MAPB, 6-APB, 6-MAPB, methylone, mephedrone, GHB, αMT, and αET, MDAI, MDPV, or others known to one of skill in the art) (10 people, 5 male 5 female), or a placebo (10 people, 5 male 5 female), on three separate occasions over the course of 3 weeks. Prior to the experiment, the participants will undergo a “wash-out” period of 10 days, wherein the individuals will cease all medication that may interact with the compounds tested within the study.

During the dosing sessions, the individuals will be monitored by a health professional for psychological and physiological indicia known to those in the art and including at least one objective measurement, as disclosed herein. At the end of each dosing session, the individuals will record or discuss their experiences with the dosing (e.g., the emotions elicited by the compound, any fears, anxieties, etc., experienced; how they felt physiologically, e.g., if they experienced nausea, a headache, a noticeable increase in heart rate, etc.). The magnitude, qualities, and/or characteristics of the entactogenic mindstate are also determined by measuring the levels of one or more of serotonin, dopamine, norepinephrine, oxytocin, and cortisol in vivo or ex vivo (e.g., in plasma), and/or by brain imaging techniques.

This information, in combination with data obtained through monitoring of at least one objective measurement, is compiled and compared between the three groups. It is determined that the entactogenic combinations are at least as effective in treating a CNS disorder as MDMA. In addition, as it relates to safety, the patients will report and it will be objectively illustrated via at least one objective measurement obtained that the entactogenic combinations have less adverse side effects than MDMA.

### Example 12: Study Determining Entactogenic Effects of the Invention

A study is to determine the subjective entactogenic effects, as compared to entactogenic compounds known to those of skill, including but not limited to MDMA. The study includes a total of 30 participants (15 male, 15 female), split into a total of three groups of ten participants each (comprising 5 men and 5 women), wherein each individual group will be administered a separate compound, one of which being a disclosed combination or composition, one being MDMA, and the other being a placebo.

There will be a total of three separate dosing sessions supervised by medical professionals to ensure participant safety, spread over a total of 30 days, (with dosing sessions every ten days) in addition to an optional “wash-out” period of from 10-60 days (e.g., 30 days) prior to participating in the experiment to ensure any/all prescription medications and recreational drugs taken by the participants prior to the dosing sessions do not significantly affect the results.

On dosing days, each participant will be administered an effective amount of the compound assigned to the group, and will be instructed to lay in a comfortable position throughout the entirety of the dosing session. The session will be recorded for audio and video, so that the participant is able to review the footage after the duration of the session.

Once each session is complete, the participant will then record their subjective experience in a journal, with an emphasis on the emotions felt by the participant during the dosing experience, and the perceptual changes experienced.

The subjective entactogenic effects will also be measured using one or more questionnaires or assessments, such as the Brief Fear of Negative Evaluation-revised (BFNE), the Authenticity Inventory, the Abnormal Mental States questionnaire, Positive and Negative Symptom Schedule-X (PANAS-X), Brief Fear of Negative Evaluation (BFNE), Visual Analog Scale Items (VAS), and Trustworthy Face Task (TFT), the Subjective Drug Effects Questionnaire (SDEQ), other another questionnaire or assessment known to those of skill (e.g., the Altered States of Consciousness Rating Scale, the Adjective Mood Rating Scale, the List of Complaints, the State-Trait Anxiety Inventory, the EWL Mood Questionnaire, the Visual Activity Scale, the Drug Effects Questionnaire, the Interpersonal Attraction Questionnaire, the Perceived Responsiveness Questionnaire, the Social Interaction Questionnaire, a Video Recording of Social Interactions, the Facial Emotion Recognition Task, the Multifaceted Empathy Test, a Movie for the Assessment of Social Cognition, Social Value Orientation, a Moral Judgment Test, the Intra/Extradimensional Attentional Set Shifting Task, a Rapid Visual (Information) Processing Task, the Severity of Symptoms Scale for PTSD, the Beck Depression Inventory, the Hamilton Rating Scale, the Modified Fear Scale, the Maladjustment Scale, the Rosenberg Self-Esteem Scale, and the Two-Choice Prediction Task).

The magnitude, qualities, and/or characteristics of the entactogenic mindstate are also determined by measuring the levels of one or more of serotonin, dopamine, norepinephrine, oxytocin, and cortisol in vivo or ex vivo (e.g., in plasma), and/or by brain imaging techniques.

At the end of the experimental period, it will be determined that the entactogenic combinations produce substantially the same entactogenic effects as MDMA, while also including expanded and enhanced entactogenic effects never having been documented before during MDMA administration.

### b. Administration Examples

### Example 13: Administration of THC and Moxonidine

An individual is administered 25 mg of THC and 0.25 mg of moxonidine, formulated as a tablet. The effects of the combination are felt by the individual around 45 minutes after taking the composition, wherein the “effects of the combination” are an entactogenic mindstate according to this disclosure. In some embodiments, the entactogenic mindstate is an expanded or enhanced entactogenic mindstate compared to a comparator entactogen such as MDMA.

### Example 14: Administration of CP 809101 and Tizanidine

An individual is administered 7.5 mg of CP 809101 and 15.0 mg of tizanidine, formulated as a capsule taken orally. The effects of the combination are felt by the individual approximately 45 minutes after taking the composition, wherein the “effects of the combination” are an entactogenic mindstate according to this disclosure. In some embodiments, the entactogenic mindstate is an expanded or enhanced entactogenic mindstate compared to a comparator entactogen such as MDMA.

### Example 15: Administration of WAY 161503 and Oxymetazoline

An individual is administered 10 mg of WAY 161503 and 1.0 mg of Oxymetazoline, formulated as a suspension taken orally. The effects of the combination are felt by the individual approximately 35 minutes after consuming the suspension, wherein the “effects of the combination” are an entactogenic mindstate according to this disclosure. In some embodiments, the entactogenic mindstate is an expanded or enhanced entactogenic mindstate compared to a comparator entactogen such as MDMA.

### Example 16: Administration of TCB-2 and Rilmenidine

An individual is administered 4 mg of TCB-2 and 1 mg of rilmenidine, formulated as a suspension taken orally. The effects of the combination are felt by the individual approximately 35 minutes after consuming the suspension, wherein the “effects of the combination” are an entactogenic mindstate according to this disclosure. In some embodiments, the entactogenic mindstate is an expanded or enhanced entactogenic mindstate compared to a comparator entactogen such as MDMA.

### Example 17: Administration of 5-MeO-MiPT and Rilmenidine

An individual is administered 8 mg of 5-MeO-MiPT and 1 mg of rilmenidine, formulated as a tablet taken orally. The effects of the combination are felt by the individual approximately 45 minutes after taking the tablet, wherein the “effects of the combination” are an entactogenic mindstate according to this disclosure. In embodiments, the entactogenic mindstate is an expanded or enhanced entactogenic mindstate compared to a comparator entactogen such as MDMA.

### Example 18: Administration of 5-MeO-MiPT and Clonidine

An individual is administered 0.2 mg of clonidine, formulated as a tablet taken orally (one 2-mg tablet, or two 0.1-mg tablets). Three hours later, the individual is administered 3-15 mg of 5-MeO-MiPT by inhalation (i.e., vaporization). The effects of the combination are felt by the individual almost immediately after the 5-MeO-MiPT is inhaled, wherein the “effects of the combination” are an entactogenic mindstate according to this disclosure. In some embodiments, the entactogenic mindstate is an expanded or enhanced entactogenic mindstate compared to a comparator entactogen such as MDMA.

### Example 19: Administration of 5-MeO-MiPT and Moxonidine

An individual is administered 0.25 mg of moxonidine, formulated as a tablet taken orally. One hour later, the individual is administered 5-10 mg of 5-MeO-MiPT intranasally. The effects of the combination are felt by the individual almost immediately after the 5-MeO-MiPT is administered, wherein the “effects of the combination” are an entactogenic mindstate according to this disclosure. In some embodiments, the entactogenic mindstate is an expanded or enhanced entactogenic mindstate compared to a comparator entactogen such as MDMA.

In several examples of such administration, the combinations as follows were self-administered, with the subjective reports recorded by the individual during and/or following such administration, as below.

### Primer and Probe: 5-MeO-MiPT and Rilmenidine

Protocol: 1.0 mg Rilmenidine and 8 mg 5-MeO-MIPT 1:30 pm

Report: “This experience was to test if 5-MeO-MIPT and rilmenidine taken together at the same time would work. The result has confirmed that it works. I swallowed 5-MeO-MIPT+rilmenidine at 1:30 PM. I then sat down listening to news. Around 2:08 PM, the transition came strong, and I could not continue listening to the news. I went to lie in the bed. The transition was mainly physical; my mind stayed mostly sunny, steady, open, with only a very brief moment of unsettlement at the end of transition.

“Throughout the experience, my stomach didn't feel good. However, even in my transition, I could tell this was a good mental state, sunny, bright, sumptuous. I couldn't tell the exact sign of my mental being clearing up, I didn't notice any mental swing but I just passed the transition. The first noticeable effect was around 2:50 PM, I commented it felt sensual, erotic, and beautiful, and I was self-centered, which meant was centering on self, didn't mean I was selfish. I was the source of the energy, and the things flew out from me spontaneously, a very healthy, natural love toward my own self, which didn't reside on the concept of love and any kind of cognition (love felt like a strong word, in truth I was in the state where love was a natural state of being). It was more like children being themselves wholeheartedly, everything was in an organic unity, undivided, a wholesome original self. I felt this experience was deeper and richer than last time when I took 5-MeO-MIPT+rilmenidine at separate times.

“It was peaceful and settling, but it came from that I was centered, so completely settling on being myself. (how nice one could just be oneself But without this kind of experience to serve a reference, we wouldn't know how much self we've compromised, and one had to strive for power and money and territory to maintain the conditions for one to be oneself, which was then hardly holding together).

“I had a children's mentality of exploring the world. I compared it with the adults' cognitive way, which was often objective and calculated, cold. Children's exploring the world was spontaneous, immediate, and for play. My exploration had a much bigger scope though. I remembered my niece used to pick up a leaf, or a stone when I took her out when she was little; everything could attract her attention. There was a natural interest. I had a natural interest too, but what I explored during the experience was different modes of feelings that I had experienced: how much I had been hijacked by those feelings and could not get me out of them. Usually the ones hijacking me were strong feelings, both negative and positive, they made such a deep impression in me that I either wanted to possess the objects that caused a great emotion or wanted to remove the objects that caused a great distress. Both were difficult, and it ended up with me being entangled with the objects.

“During the experience, it was easy to see this, since I was safe and solid in my center, in a safe and solid world; I wasn't tangled. All those entangling objects were just like my niece's leaf or stone, I picked it up, and had a deep experience with it, then moved forward. It was me experiencing them, instead of being hijacked in their loop. This was a very insightful period. I had some deep revelations here, all because my centering on myself created a great reference point to contrast what might be the problems in the everyday life. They started to become an experience, not my whole life. It made it safe for me to experience deep feelings, without the danger of being hooked in it and losing myself.

“I also recognized that deep emotions came from innate desires, and innate desires were real and human truths. So I would still delve into deep experience, let myself become lost in it, and what I needed was to be able to restore myself at a certain point. This combination of 5-MeO-MIPT+rilmenidine could help me. I was pleased, but my stomach still felt bad. I thought some food might help. It was around 3:30 PM. I ate a few fruits, and went to tell my partner this was a great experience. We went out to the garden. It was nice, but we didn't stay long. I was very much engaged in a brief talk with my partner, which was rather scientific, but I didn't approach it in a scientific way, it was quite concrete, sort of discussing rearranging something real in a different way. I wasn't able to think of the world through conceptual or abstract methods. It was incompatible, too pale.

“Around 3:40 PM, I desired for a watermelon, then I suggested to go to a store to buy it. It was such a fun idea and I was excited. This was unusual. I never wanted to go to the public during an experience, and probably never had done it. However, it felt such a fabulous thing to do. So we went out. Along the way, I was suddenly stricken by a thought “what if I lose control of myself. I am in an experience after all and maybe I can't tell if I behave ok.” I was somewhat panicked at the idea, and consequently the street appeared a bit menacing. Here I had a brief period of cognitive reasoning with myself: I've never lost control, I can function normally, etc. But it didn't work, I was in a bad worrisome mood. Then I thought: what the heck, if I lose control, just lose it! That seemed to settle it. I was again looking forward to the store, but I did feel the uneasiness hanging around me.

“In the store, I was also aware of my condition. Sometimes I would check myself self-consciously, but most of time I forgot it. It was fun shopping. My desire was straightforward and prompt. I wanted a watermelon, a crunchy and chewy loaf of bread, some cheese and that led to buying grapes. I was drawn into picking things, and often lost in details. Everything was what I wanted, so it was great.

“We got home around 4:30 PM. I ate the watermelon, the bread, the cheese, the grapes; even though cookies tasted good, I didn't care for them. I was more into organic earthy taste.

“Afterwards I went to lie in my bed. It was a very loving period. I started to feel people, feeling them as human beings; I didn't judge, and I was effortlessly empathetic. Genuinely loving. Before this, I was centering on myself experiencing things; now I started to feel people, the mental space was quite populated, each was vivid and substantial; what I sensed was the inner beings. Then I went to watch a long movie.”

### Primer and Probe: 5-MeO-MiPT and Rilmenidine

Protocol: 1.0 mg Rilmenidine 3:00 pm; 8.0 mg 5-MeO-MIPT 4:30 pm

Report: “I had a lunch of two buns around 12:45 PM. At 3 PM I took 1 mg rilmenidine. At 4:30 PM I took 5-MeO-MIPT 5:05 PM I felt a bit cold, transition began, some abdominal pain like some gas was trapped, on the right side. Transition was much more uncomfortable and intense than with citronellol. Vague close-eye pattern, not much mental energy, sedated, passively transitioning, not much volition. Quite cold. Further close-eye vision, a pattern like symmetric silhouette of pea pods against black background, with beads flowing orderly through pods. Shadowy statues.

“6:01 PM, started to clear up, felt better, sensing more flow. Right away I was relaxed, and this relaxation was like being released from inner clench, which reminded me that I was usually not relaxed, wound in knots. I started to wonder if I usually have a psychological clench, and if my niece, being so young, had a psychological clench. I thought I was responsible for my niece's psychological clench, and wanted to do better for her. I could feel both my niece and my mother by sensing their emotional states. My mother had a rich foundation of feeling, supple and with great energy. She had a hard life but still steady like a rock, and I wanted so much to develop a much deeper and entwined relationship with her.

“It appeared that I was more in direct contact with emotional states, instead of judging this or that behavior, or what they had said. Behaviors were one thing, restricted by conditions and scope and one's knowledge, but one can build great inner characters through any way of doing. What I sensed was inner characters. My mother was rich while my niece's character was thinner and still unsteady. I had a lot of concern toward my niece. Some music was playing on TV. It was immensely beautiful. From this point on, I was in a completely relaxed state, in a sunny beautiful world, as though I was sitting on a meadow bathing in sun. I was a transparent sensing being, fully open, no clutter, no clench, no intense euphoria, but immersed in a well-being so divine and a joy so simple so natural that I actually thought I was in a state of fully human. (divine and human went together, yes) It was centered on self but not selfish at all.

“Then by accident I peeked at the TV screen, and it happened to be showing some weird commercial [anti-smoking]: an old scrawny cadaverous [cancerous] woman sitting on a chair talking, her cheek hollowed, something eating her away, her decay was so striking that it hit me like a force. Even though I turned away right away, the image stuck with me for a while, carrying a pure and unsettling aftertaste. However, even though it was so striking, and it did incur a brief period of cascading thoughts of this category of human condition, and I was very uneasy as it reminded me of my sick family, the sense of well-being was so pervasive that it provided a floor for it. There was no moment I was rejecting it, I was prone to thinking along the line of providing care and preserving dignity. This was quite empathetic.

“Likewise, I was less concerned while thinking of my niece's likelihood of not passing the exam to get to the school we wanted her to. There was also an emotional floor beneath her propping and nurturing her and she didn't appear so fragile, and also it shrank the issue of exam to a more proper proportion. The fixation was untwined. It was as though I thought a lot, but in fact I wasn't. All these thoughts came and went quickly, as I appeared to be directly seeing the nature of the problems and they were so clear and so the response arose directly and there was no fuss.

“This combination was more like providing a platform to view things. Most of the time, I just enjoyed this wholesome, relaxed, divine loving mental state of total well-being. It was something a child could experience, when this undivided well-being still felt like a natural law and a universal principle, more objective than subjective, and the being was still uncluttered, unburdened, open and transparent, walking through an equally open world. It was exquisite and spiritual and somewhat artistic but not overwhelmingly so. I had a memory that felt like a reliving: I was a child walking through the street with my grandfather; I was effortlessly absorbing what's going on in the street. It was a memory but it didn't have the distance of a memory, it was more like a reliving in my grandfather's presence.

“A movie was playing. It had a nature scene of lava pouring down mountain, and human faces with impressive emotions. Probably it took efforts to be grand or to be impressive, I wasn't captivated by it at all. I even found the lava pouring scene very small, not grand at all. All in all, I was in a totally humanized world, it was divine, but not grand. The size had to fit this humanized terrain.

“Also I noticed the visual input seemed distant while sounds, smells and tastes were immediate. Sounds, smells and tastes could grab me immediately while I was less responsive to visuals. Music was immensely beautiful, flowers smelt so good, and food was delicious.

“My partner asked me if I felt innocent. I didn't feel innocent. But I wondered why we couldn't live in this state, which made wars so unnecessary and ridiculous, but I certainly knew why we had war. One moment I checked my phone, and saw news on geopolitical tensions currently going on. I had been very troubled by it [over the past two months], and quite taken over by it. But at this moment, it seemed unable to coexist with my mental state. It was pushed to a corner, small and trivial; all those big political figures threatening to do this and that were medieval too. However, I also remembered that when I was taken over by those issues (for two months I studied them), they looked enormous and there was no feeling of well-being. It was a fixation. When I examined it further [today?], I thought I didn't have to fix myself on it, for it didn't make up my whole world, I could exercise this well-being with the people I care about, instead of being fixed in the issue I could not control, even though they were indeed scary and would affect us all, however, the world had always been like this, and we probably could not escape our limitation. [above was in peak state]. I desired to watch again a German film, but we did not have it. We then watched an American movie. The story wasn't interesting, but the small town felt like an entity, a space filled with personal history and entanglement, and it was great to sense a humanized world. It was not a great movie though. The German film would be the perfect fit, because it also had exquisite and artistic aspects that would resonate better with the experience.

“This is the fourth day. I am still in the state, in fact, seems even steadier now. The second day I was a bit down, tired because I had to write the report; but the third day I went back full blood, and now the fourth day it remains: wholesome, well-being, porous, no clutter, no clench, no fixation, zest for life.

“In this state the center is self. Rilmenidine connects to self. When one connects to self, anxiety, guilt and fixation smooth out. One is self-centered, there is no need of belonging somewhere, no such thinking (but I am comparing, this state is again a reference point to other mental states). External things can be picked up easily and become my inner music, so there is no sense of gap. I am also aware that my past is not just a memory, not so remote and disconnected as I usually feel. The past (good and bad) is a part of an inseparable whole as I am feeling now, so it is immediate and intimate. This sense of self is more realistic. I am healthy, whole, feeling my inner strength again.”

The following example was performed for purposes of and is presented as a control.

### Compound: Rilmenidine Alone

Protocol: 1.0 mg Rilmenidine 9:00 pm

Report: “I took a pill of rilmenidine at 9 PM. I felt its effect within half an hour. I was quite stressed before the pill, so even though the effect was subtle, I detected it. It was relaxing, my chest less strained, my usually tired eyes also relaxed (I wondered if my eyes being so tired might in some part come from stress). Around 11 PM, I felt that the effect was less obvious. I took a tiny bit Ambien for sleep.”

### Example 20: Administration of 5-MeO-MiPT and Moxonidine

An individual is administered 8 mg of 5-MeO-MiPT and 0.2 mg of moxonidine, formulated as a tablet taken orally. The effects of the combination are felt by the individual approximately 45 minutes after taking the tablet, wherein the “effects of the combination” are an entactogenic mindstate according to this disclosure. In some embodiments, the entactogenic mindstate is an expanded or enhanced entactogenic mindstate compared to a comparator entactogen such as MDMA.

In an example, the combination as set forth below was self-administered, with the report as follows.

### Primer and Probe: 5-MeO-MiPT and Moxonidine

Protocol: 0.2 mg Moxonidine and 8 mg 5-MeO-MiPT 1:00 pm

Report: “An extraordinary experience. At 1 PM, I swallowed 8 mg 5-MeO-MiPT and a 0.2 mg pill of moxonidine. I didn't experience any physical discomfort, completely lacking a body load, meaning, there was no detectable transition phase. Only probably around 2:19 PM, my thinking seemed a bit off, but it was very mild. Throughout the whole experience, I didn't experience any of my usual physical discomfort. (This might be a good candidate for therapeutic use if such a light transition can be repeated.)

“Because of lacking the physical discomfort, I wondered if I would have an experience, and what might cause this combination to not have an effect on me. Around 2:25 PM, I felt a weight in (in, not on) my chest. The mental space opened up, I started to mentally connect to a large space. Now I knew I was going to have an experience, and it was very promising.

“Right away, I felt purposeful and meaningful, and this was not the dramatic or chemical or religious kind of purpose or meaning. It was deep and nuanced, within me. It was a natural heart opening to my surroundings, which was not specific to one location, but a general orientation toward the world and one's personal life. Right away, I knew I hadn't been in this state for many years, and right away I knew I was out of a familial grid for many years. This heart state was the one with which I used to move through my personal world, that took root on solid and subtly active familial settings. You effortlessly moved through, your heart opened to it, you picked up the invisible subtle cues that were not static, but ongoing in a real-time manner, and there was resonance all around, which was natural and nothing dramatic, a giving that you didn't need even to notice. (I noticed it because I was off from it for so many years.)

“I was so completely centered on my heart. It was such a solid heart space. I could still think, but since now the center of my consciousness was the heart, feeling and thinking was aligned, and I had no body load; I hardly thought it was a drug experience; there was no distraction. It looks like heart opening has different degrees of openness, and this one was completely open, so open that I felt I had moved to the center of my being. I was deeply touched. This touch was both physical and affective: there was weight in my chest, there was the state of emotion. I was often in tears. The feeling was complex, but it was not overwhelming, it was quietly lingering in my chest, very gentle, but carried a depth.

“I knew my place in the world and what had built my heart. It was gratifying to know. This knowing was direct—you knew or not knew. It was quite different from the knowledge through analysis. We can imagine how a child moves through the world: completely heart-centered, effortlessly engaging and resonating, within an invisible web of heart-absorbed associations.

“At this period of time my feeling was more connected with my family. I remembered how we used to associate with each other, and how it was lost. And I felt guilty about it, I believed I was the one who pulled out and had been judgmental toward them. I also felt sad that I wasted so many years not living in my heart space, but distracted by things that really didn't matter to me much. When disconnected, it is easy to feel drifting in a rootless space. I was very easily in tears.

“But even though I was more emotionally connected with my family, I was in an opened-up space. It was like the time before we start to guard our hearts therefore isolating and disconnecting us from the large world and other people. The lack of guard was probably equal to the openness of the heart.

“This part about “guard” was a reflection that came later. During the thick of experience, I wasn't very reflective, but along the line of “know” or “not know”. I also didn't ponder on “wholeness”. Both came later when I was a bit down from my experience, so my cognition came in, starting to reflect on the state.

“I did have some memories. These memories were not like the normal times we remembered a scene or something, your mind focused on them locally and narrowly. Here the memory was more like entered the setting again, and experienced all the details as they unfolded—it had a process.

“In this state, I was an individual rooted on the heart that was built from all the relationships that had touched it. I was connected to the world as a whole, so I had a robust self-centered outlook. Around 3 PM, my mind shifted to my real-time setting. It felt spacious, bright. I didn't have much thinking.

“I felt giving was a delightful act, especially to the younger ones in my family. It was not about gifts, it was the act and the intent that infused that act. I remembered my grandparents used to give or do many things for me. It was a natural expression of affections. I felt affectionate toward the kids. And this seemed to be the first time I felt this more mature and elderly affection toward the kids: protective, benign, delightful at giving something that might be trivial to the kids. I wasn't thinking of lavish gifts—it was not about gifts, it was the act of giving, and my intent which was filled with caring for the young ones.

“At one moment, I listened to a YouTube program talking about world affairs. It went back to 100 years ago, and moved across the globe, but both the time and location felt close, as though I was listening to a neighbor talking about what had happened inside another neighbor's household. It felt like all events happened under the sun, so no matter how bizarre or tragic, they belonged to a large comprehensible whole. This unity removed strangeness and added depth.

“Another moment, I was lying there. I realized everything I really loved was free, like sunlight, breeze, a girl's smile, whatever came to me, it carried a concrete moment with a concrete setting: sunlight on me, breeze outside the latticed window, and a girl looked up smiling at me. All real.

“There was no hangover afterwards. 1st day after: slept fine. I woke up carrying some of yesterday's mood, and surrounding more connected. No hangover. 2nd and 3rd days after: normal state. It appears that the aftereffect didn't last as long as rilmenidine.”

The working example below was performed for purposes of and is presented as a control.

### Compound: Moxonidine Alone

Protocol: 0.2 mg Moxonidine 12:30 pm

Report: “Took one pill of Moxonidine at 12:30 PM, in the backyard bathing in the sun. At about one hour, I felt more focused, probably more centered, less distracted by thinking, more situated in the body. Not reflective, more like just being there. All very subtle but detectable.”

The working examples below were also performed for purposes of and are presented as controls, using an escalating dose of a single primer alone, applicable to this and other Examples.

### Compound: 5-MeO-MiPT Alone

Protocol: 4.0 mg 5-MeO-MiPT 1:09 pm

Report: “Written during the session. I took it at 1:09 PM. Then I sat in the couch reading a book. At some point, I felt some sensation at the root of my teeth, which usually happens during transition. It was very brief. Then I felt cold, so I wrapped myself up in a blanket, lazily half-lying in the couch continuing the book. It was ok to read. Then I felt tired and closed my eyes to rest; my brain was so heavy and tired that it felt like I was going to get knocked out or fall asleep, and I had no energy to think of anything. My brain seemed to want to lay there dormant, but I opened my eyes trying to read again, and reading was ok. It appeared that when there was no sound in my surroundings, the silence was more salient than usual.

“Then I went to lie down in the bed. The music was playing, but it didn't do much to me. The music felt quite normal. Some point between 2 to 2.5 hours, the heaviness in my brain lifted, and things have been quite clear since. My brain is clear and lucid. We went out to the garden. I really enjoy the garden in the bright day light. Today is a sunny day, a very beautiful day. If not for the mosquitoes, I would like to stay out, just sit there. My emotion is quite neutral, not much different from the normal state.

“I thought of my cousin. I think recently I point out his weakness too often. It's not nice. He is a very good person, has a soft heart, seems more sentimental than me. I was sentimental before, but I do not care for sentiments too much now. My cousin still retains emotional nuances. I think I might have reached an age that I should start the second half of life. The focus of this part of life is different from the first part. The first part is more dependent and emotional; the second part is more independent, free, and mature.

“It's very nice to feel my brain clear, smooth, and present. I am quite relaxed.”

### Compound: 5-MeO-MiPT Alone

Protocol: 6.0 mg 5-MeO-MiPT 3:30 pm

Report: “Taken at 3:31 pm. A slight bit of physical sensation at 4:40 PM. At 5:57 PM, I feel my mind is clear, and I don't have those bad thoughts that keep my mood down and me awake in the night. I feel peaceful. Sometimes I feel I don't have energy to think. At 6:18 PM, look around the garden. I have my glasses on, and the garden looks great. I like being outside. As last time I feel my sight cleared up. It has nothing to do with glasses. Generally, my brain is clear.

“It is very subtle. I don't have feelings associated with the news in TV, less than usual; their facial expression has no effect on me. I am generally emotionally neutral. Overall, I like this drug. A light lucid clear brain seems nice. I feel my hands (fingers while writing) are lighter too.

“(This can be a good drug for conversation. I had the same feeing last time: lucid mind, relaxed, unobtrusive, and a very even mood. Not for argument, but a leisurely conversation can be fitting.)

“At 6:31 PM, I feel this all along—my brain is in a cozy, clear, light state.

“At 7:49 PM, I am probably down. Sex was very good. I felt I was melting under caress, very enjoyable. My mind was in and out of sex, neutral, and sex free, didn't have a lot of thoughts.

“I had something going on with my cousin. I was upset before the experience. But now I calmed down, and could look at the problems from different angles, realizing that the most important thing was not money, but my cousin. Whatever happens, he should be the center of my concern, not the money. Life is long, and the loss can be earned back. Even if it doesn't, that loss doesn't really reduce the quality of life.”

### Compound: 5-MeO-MiPT Alone

Protocol: 8.0 mg 5-MeO-MiPT 3:00 pm

Report: “I took the drug at 3 PM. It is 7:35 PM now. At this moment I am very much out of its effect.

“I took the drug and went to take a shower. I felt its onset during the shower. It came on strong. I was very introspective, thinking that I liked the solitude aspect of life, and everybody was a lone individual on this planet, and each had their own destiny; no matter how much help they could get, they still had to go through their individual life. I had a mental image of my nieces: they were very distinct, more distinct that the usual mental images I would have about them. They had more being, a lot of soul, with lots of details.

“During the transition, my body was uncoordinated, and I trembled a lot. I didn't have much mental activity. There was no emergence of a stable mental state. It was in fact more like the dissolution of my mental states. 1.5 to 2 hours after taking the drug, I felt that my mind became transparent, and the boundary was dissolved. I was a bit afraid, because I felt this kind of dissolution was like the one that might come from ketamine. But there wasn't much mental activity, and I couldn't engage in serious thinking, which was good, because in this way I had no ability to become paranoid. Actually, it was kind of cozy, and occasionally I even had a homey feeling. I felt my brain as a boundless space with nothing going on—my face a disproportionately thin surface while my brain case was disproportionately big with a lot of space.

“Music could come right through my mental space. I totally didn't feel the weight of my brain, which had no boundary. I also didn't feel much of myself. I felt very light, sometimes weightless, floating. I was inside the space of the music. I wasn't judgmental toward music at all. It all seemed just some sound. There was not much feeling. Music permeated my mental space, and I couldn't discern the boundary between my mental activity and the music. I also felt my physical body was fuzzy. I said I was warm, soft. It wasn't about my body, it was about my material. Sometimes I said something, and I felt the word come directly from my throat to my mouth, and there was no mental activity associated with the utterance of the word.

“We went to another room. Everything had its own essence. I noticed their presence more. They had silent, friendly being. The vase was a vase by nature, not about the painting on its surface. The flowers looked livelier and beautiful. The carpet was not flat, some bits higher and some lower. Visual was closer to adrenergic kind, very subtle. My eyes were clear, as though I could really open my eyes to see things.

“(Extra comments: I slept well after all these experiences. It cleared my mind, and I usually was more peaceful and relaxed. I have come to believe this drug can help me sleep.)”

### Compound: 5-MeO-MiPT Alone

Protocol: 10.0 mg 5-MeO-MiPT 2:00 pm

Report: “Written at 6:45 PM, still feel the effect. I took 10 mg 5-MeO-MIPT at 2 PM.

“It came on fast and strong in 30 minutes. I went to the toilet (it also made me go to the toilet last time) and stayed there for maybe 15 minutes. I noticed there was some movement in the rug, which appeared not all flat. My stomach felt heavy. I came out of the toilet at 2:37 PM. I noticed the carpet had movement too. My stomach was so upset that I had to vomit. Afterwards I felt better. I sat on the floor examining the carpet. It was rippling, waving, and (maybe) had a slight patterning. The vision was definite but it was milder and slower than in 2C-B.

“Then we went to a back room. Very soon, I went into the same state as the last trip. I felt weightless. I could not portray myself in my mind. I believed that if I looked at the mirror, I was so transparent that I could not find myself in the mirror. I was void of emotion and mental activities. The latter part about being void of mental activities was extraordinary because meanwhile I found that I could engage in talking, and describe my own mental state. This mental state was quite remarkable.

“I felt like I was a transparent being, and everything could come through me: the sunlight, the thread of the music. I totally didn't have emotion attached to any music, but they presented a more spacious, more complex space, which felt more interesting. The music was omnipresent, like we might experience in an omni theater. It's more pronounced with eyes closed. I don't think I was absorbed into it. I was pretty neutral and just passing though. My body was weightless, but I could feel some feeling passing through my body when I was totally relaxed. It was so comfortable. I enjoyed it very much. I think it's precious in a way that was unburdened of both my matter and mind, just a sheer being, neutral, nonjudgmental, weightless, totally bathed in whatever the space brought me. My self didn't weigh much, and being so transparent I felt boundless. (I want to make a note here that it wasn't a zombie-like state.)

“When I looked at things, the details seemed to try to present themselves so that I could not miss them. They looked very individual and beautiful. At one moment when I looked at this yellow leaf, for fleeting seconds I felt it was creating its own dimension of space (like the one I had in DOI), and I was in ancient Japan—even though I was in the same room and the same setting, but it was a totally different association, and everything was transformed into a different meaning; I myself was all present in those fleeting seconds. This was after I was a bit past the peak state and I began to feel my body.

“We bicycled to a nearby school. A lot of people were out enjoying their time. It was such a beautiful autumn day. It was warm. The leaves glistened under the setting sunlight. Perfect. I totally liked what I saw. But meanwhile I noticed that I didn't truly feel much. I felt the kind of happiness that belonged with other people, while I was a neutral observer, and very much liked whatever I saw. At the time of writing, I feel my mind is very clear, and smooth, as though I had the effect of a low dose of this drug.”

### Example 21: Administration of DALT and Naphazoline

An individual is administered 60 mg of DALT and 1 mg of naphazoline, formulated as an injectable solution. The effects of the combination are felt by the individual almost immediately after the formulation is injected, wherein the “effects of the combination” are an entactogenic mindstate according to this disclosure. In some embodiments, the entactogenic mindstate is an expanded or enhanced entactogenic mindstate compared to a comparator entactogen such as MDMA.

### Example 22: Administration of CPP and Tetryzoline

An individual is administered 12 mg of CPP and 100 μL of tetryzoline, formulated as eye drops. The effects of the combination are felt by the individual within about 10 minutes after applying the formulation to the eye of the patient, wherein the “effects of the combination” are an entactogenic mindstate according to this disclosure. In some embodiments, the entactogenic mindstate is an expanded or enhanced entactogenic mindstate compared to a comparator entactogen such as MDMA.

### Example 23: Administration of 1-Methylpsilocin and Tizanidine

An individual is administered 12 mg of 1-methylpsilocin and 1 mg of tizanidine, formulated as a sublingual tablet. The effects of the combination are felt by the individual within ten minutes of placing the sublingual formulation beneath the patient's tongue, wherein the “effects of the combination” are an entactogenic mindstate according to this disclosure. In some embodiments, the entactogenic mindstate is an expanded or enhanced entactogenic mindstate compared to a comparator entactogen such as MDMA.

### Example 24: Administration of DOB and Rilmenidine

An individual is administered 2 mg of DOB and 1 mg of rilmenidine, formulated as a buccal tablet. The effects of the combination are felt by the individual within ten minutes of administering the buccal tablet, wherein the “effects of the combination” are an entactogenic mindstate according to this disclosure. In some embodiments, the entactogenic mindstate is an expanded or enhanced entactogenic mindstate compared to a comparator entactogen such as MDMA.

### Example 25: Administration of NBOH-2C-CN and Guanfacine

An individual is administered 1 mg of NBOH-2C-CN and 2 mg of guanfacine, designed as a topical formulation. The effects of the combination are felt by the individual about 60 minutes post-administration of the topical formulation, wherein the “effects of the combination” are an entactogenic mindstate according to this disclosure. In some embodiments, the entactogenic mindstate is an expanded or enhanced entactogenic mindstate compared to a comparator entactogen such as MDMA.

### Example 26: Administration of Rilmenedine, THC, and DOET

An individual is administered 20 mg of THC, 5 mg of DOET, and 1 mg of rilmenidine, wherein the THC is administered as a an oral tablet, and the rilmenidine and DOET are formulated in an injectable solution. The THC is consumed approximately 45 minutes prior to the injection of the rilmenidine and DOET formulation. The effects of the combination are felt by the individual nearly immediately after the formulation is injected, wherein the “effects of the combination” are an entactogenic mindstate according to this disclosure. In some embodiments, the entactogenic mindstate is an expanded or enhanced entactogenic mindstate compared to a comparator entactogen such as MDMA.

### Example 27: Administration of Moxonidine, THC, and TCB-2

An individual is administered 20 mg of THC, 4 mg of TCB-2, and 0.25 mg of moxonidine, wherein the THC is inhaled as a vaporized liquid, and the moxonidine and TCB-2 are formulated as a transdermal patch. The transdermal patch containing the TCB-2 and moxonidine is placed on the patient roughly 60 minutes prior to inhaling the THC formulation. The effects of the combination are felt by the individual nearly immediately following the inhalation of the THC formulation, wherein the “effects of the combination” are an entactogenic mindstate according to this disclosure. In some embodiments, the entactogenic mindstate is an expanded or enhanced entactogenic mindstate compared to a comparator entactogen such as MDMA.

### Example 28: Administration of MEM and Moxonidine

An individual is administered 30 mg of MEM and 0.25 mg of moxonidine, formulated as a liquid for vaporization. The effects of the combination are felt by the individual nearly immediately following the inhalation of the formulation, wherein the “effects of the combination” are an entactogenic mindstate according to this disclosure. In some embodiments, the entactogenic mindstate is an expanded or enhanced entactogenic mindstate compared to a comparator entactogen such as MDMA.

### Example 29: Administration of DOB and Rilmenidine

An individual is administered 1 mg of DOB and 1 mg of rilmenidine, formulated as an injectable solution. The effects of the combination are felt by the individual almost immediately after the formulation is injected, wherein the “effects of the combination” are an entactogenic mindstate according to this disclosure. In some embodiments, the entactogenic mindstate is an expanded or enhanced entactogenic mindstate compared to a comparator entactogen such as MDMA.

### Example 30: Administration of 2C-B-FLY and Clonidine

An individual is administered 8 mg of 2C-B-FLY and 0.2 mg of clonidine, formulated as an injectable solution. The effects of the combination are felt by the individual almost immediately after the formulation is injected, wherein the “effects of the combination” are an entactogenic mindstate according to this disclosure. In some embodiments, the entactogenic mindstate is an expanded or enhanced entactogenic mindstate compared to a comparator entactogen such as MDMA.

### Example 31: Administration of 5-MeO-DMT and Clonidine

An individual is administered 0.2 mg of clonidine, formulated as a tablet taken orally (one 0.2-mg tablet, or two 0.1-mg tablets). Three hours later, the individual is administered 3-15 mg of 5-MeO-DMT by inhalation (i.e., vaporization). The effects of the combination are felt by the individual almost immediately after the 5-MeO-DMT is inhaled, wherein the “effects of the combination” are an entactogenic mindstate according to this disclosure. In some embodiments, the entactogenic mindstate is an expanded or enhanced entactogenic mindstate compared to a comparator entactogen such as MDMA.

### Example 32: Administration of 5-MeO-DMT and Moxonidine

An individual is administered 0.25 mg of moxonidine, formulated as a tablet taken orally. One hour later, the individual is administered 5-10 mg of 5-MeO-DMT intranasally. The effects of the combination are felt by the individual almost immediately after the 5-MeO-DMT is administered, wherein the “effects of the combination” are an entactogenic mindstate according to this disclosure. In some embodiments, the entactogenic mindstate is an expanded or enhanced entactogenic mindstate compared to a comparator entactogen such as MDMA.

### Example 33: Administration of 5-MeO-DMT and Rilmenidine

An individual is administered 1 mg of rilmenidine, formulated as a tablet taken orally. One hour later, the individual is administered 6-15 mg of 5-MeO-DMT intranasally. The effects of the combination are felt by the individual almost immediately after the 5-MeO-DMT is administered, wherein the “effects of the combination” are an entactogenic mindstate according to this disclosure. In some embodiments, the entactogenic mindstate is an expanded or enhanced entactogenic mindstate compared to a comparator entactogen such as MDMA.

### Example 34: Administration of 5-MeO-DMT, Clonidine, and THC

An individual is administered 0.2 mg of clonidine, formulated as a tablet taken orally (one 0.2-mg tablet, or two 0.1-mg tablets). Two and a half hours later, the individual is administered THC, formulated as a liquid (e.g., a distillate, a concentrate, or extract) for inhalation. A half hour after the THC administration, the individual is administered 3-10 mg of 5-MeO-DMT intranasally. The effects of the combination are felt by the individual almost immediately after the 5-MeO-DMT is administered, wherein the “effects of the combination” are an entactogenic mindstate according to this disclosure. In some embodiments, the entactogenic mindstate is an expanded or enhanced entactogenic mindstate compared to a comparator entactogen such as MDMA.

In each of the prophetic administration examples above, after administration of an effective amount of the entactogenic combinations, the individual administered said combinations will describe at least one feeling/emotion associated with an entactogenic experience, including but not limited to those of ecstasy, empathy, openness, compassion, peace, acceptance, healing, open-hearted tenderness, love, oneness, and caring; forgiveness, both of another (the true letting go in one's heart of anger and grudges) and of one's self (the true letting go in one's heart of guilt and shame); and the ability to safely experience and contemplate mental pain.

### R. Primer/Probe Identification

### Example 35: Exemplary Method of Determining Primers and Probes

To determine what compounds are efficacious in acting as primers or probes, chemicals were gathered from known sources in the art, including patent lists, research work, repositories, etc., and then the chemicals (sourced as chemical names) were converted to structures using Pubchem API and the Chemical Identity Resolver service provided by NIH/CADD.

Standardization of chemical structures was minimal, with stereochemistry being preserved, while counter-ions of salts were stripped. A total of 927 chemical names were converted, with 924 successfully converting (99.67%). The 924 unique chemical structures were then generated as InChlKeys, to be used as unique identifiers in comparison with other datasets containing chemical structures.

The bioassay data tables for target receptor systems (5-HT_{2A}, 5-HT_{2B}, 5-HT_{2C}, 5-HT₇, I₁, CB₁) were downloaded. Each row in these six tables contains an experimental outcome (k_{I}, IC₅₀, EC₅₀, etc.), for each compound assayed against the above target receptor systems. SMILES strings were converted to structures, and used to generate InChlKeys for the compounds found in the ChEMBL datatables.

The ChEMBL datatables were then compared to the aforementioned 924 compounds generated via the patent lists, research works, repositories, etc. using InChlKeys. The results indicated that 103 patent list compounds were found in one or more of the six tables. Herein, this set is referred to as the “103pat set.”

ChEMBLIDs for the 103pat set are then used to query ChEMBL, and the resultant data table download contains all bioassay data present in ChEMBL for each compound in the 103pat set. It is discovered that some compounds widely available in pharmaceutical compositions contain a vast number of associated bioassays (clonidine, for instance, is found to have over 1,000 different associated bioassays), while the majority of species has under 100.

Prioritization scores are then calculated for the compounds in 103pat. Averaged pKi values are calculated from Ki values for each receptor/target where Ki data is available. The number of targets each compound is assayed against are counted. In order to estimate selectivity of each compound at the 6 targets, the relative affinity is measured between the highest pKi value obtained for each compound and their pKi values at each of the six key targets. Broadly, three indices may help guide prioritization and selection of exemplary agents: (a) Relative affinity difference between key pKi value and max pKi value; (b) Count of targets compound that has been assayed against; (c) Bsq (square root of sum of squares of normalized pKi values). The process outlined above is then completed to discover the highest affinity ligands (lowest Kᵢ values) from PDSP for CB₁, I₁, 5-HT₂, and 5-HT₇.

Chemical structures for compounds from both ChEMBL and PDSP (The NIMH Psychoactive Drug Screening Program) are then clustered on the basis of chemical similarity (chemical descriptor profiles).

To ensure exemplary agents represent as many chemotypes with desired biochemical properties as possible, selection for exemplary lists is completed by identifying the most selective compounds in each cluster per group of compounds with binding data for one of the target receptors. After identifying top compounds within clusters, the initial list of exemplary agents is reviewed to fill perceived gaps in the new lists. In order for a compound to be selected as an exemplary compound, in addition to selectivity, the compound also needs to have an online record (with Wikipedia or the like as a minimum) of being observed to be an agonist at the necessary targets (except in the case of 5-HT₇).

The foregoing description, for purposes of explanation, uses specific nomenclature to provide a thorough understanding of the invention. However, it will be apparent to one of skill that specific details are not required in order to practice the invention. Thus, the foregoing description of specific embodiments is presented only for purposes of illustration. It is not intended to be exhaustive or to limit the invention to the precise forms disclosed; obviously, many modifications and variations are possible in view of the above teachings. The embodiments were chosen and described in order to best explain the principles of the invention and its practical applications, through the elucidation of specific examples, and to thereby enable others skilled in the art to best utilize the invention and various embodiments with various modifications as are suited to the particular use contemplated, when such uses are beyond the specific examples disclosed. Accordingly, the scope of the disclosure shall be defined solely by the following claims and their equivalents.

### REFERENCES

1. 4 types of creativity (voynetch). Voynetch. (2021). Retrieved Nov. 18, 2021, from https://voynetch.com/8053.
2. Abdalla, C., Krystal, J., Duman, R., Sanacora, G. (Feb. 27, 2020). PCT Pub. No. WO 2020/041329 A1, “Combination therapy for treating or preventing depression or other mood diseases”
3. Adamson & Metzner. (1988) The nature of the MDMA experience and its role in healing, psychotherapy and spiritual practice. ReVision, 10(4), 59-72
4. Alm, P. A. (2004). Stuttering and the basal ganglia circuits: a critical review of possible relations. Journal of communication disorders, 37(4), 325-369.
5. The Alternative Uses Test. Creative Huddle. (2021). Retrieved Dec. 7, 2021, from https://www.creativehuddle.co.uk/post/the-alternative-uses-test.
6. Amoroso, T. (2019). The spurious relationship between ecstasy use and neurocognitive deficits: A Bradford Hill review. International Journal of Drug Policy, 64, 47-53.
7. Anonymous (2013). An illegal drug helped me stop my habit of 19 years. Skin Pick. Archived at https://web.archive.org/web/20130621062545/https://www.skinpick.com/node/3256).
8. Backman, M., E. & Tuckman, B., W. (Jan. 1, 1972). Review of Remote Associates Test. Journal of Educational Measurement. 9 (2): 161-162.
9. Baggott, M. J., Kirkpatrick, M. G., Bedi, G., & de Wit, H. (2015). Intimate insight: MDMA changes how people talk about significant others. Journal of Psychopharmacology, 29(6), 669-677.
10. Barac, Y. D., Bar-Am, O., Liani, E., Amit, T., Frolov, L., Ovcharenko, E., Angel, I., Youdim, M. B., & Binah, O. (2012). I1 imidazoline receptor: novel potential cytoprotective target of TVP1022, the S-enantiomer of rasagiline. PloS one, 7(11), e47890. https://doi.org/10.1371/journal.pone.0047890
11. Bard J A, Zgombick J, Adham N, Vaysse P, Branchek T A, Weinshank R L (November 1993). “Cloning of a novel human serotonin receptor (5-HT7) positively linked to adenylate cyclase”. The Journal of Biological Chemistry. 268 (31): 23422-6. doi:10.1016/S0021-9258(19)49479-9
12. Baumann, M. H., Clark, R. D., & Rothman, R. B. (2008). Locomotor stimulation produced by 3,4-methylenedioxymethamphetamine (MDMA) is correlated with dialysate levels of serotonin and dopamine in a rat brain. Pharmacology Biochemistry and Behavior, 90(2), 208-217.
13. Bedi, G., et al., Effects of MDMA on sociability and neural response to social threat and social reward. Psychopharmacology (Berl), 2009. 207(1): p. 73-83.
14. Bilkei-Gorzo A. (2012). The endocannabinoid system in normal and pathological brain ageing. Philos. Trans. R. Soc. Lond. B Biol. Sci. 367, 3326-3341. 10.1098/rstb.2011.0388
15. Bosier B., Muccioli G. G., Hermans E., Lambert D. M. (2010). Functionally selective cannabinoid receptor signalling: therapeutic implications and opportunities. Biochem. Pharmacol. 80, 1-12. 10.1016/j.bcp.2010.02.013
16. Bousquet, P., Hudson, A., Garcia-Sevilla, J. A., &amp; Li, J.-X. (2019). Imidazoline receptor system: The past, the present, and the future. Pharmacological Reviews, 72(1), 50-79. https://doi.org/10.1124/pr.118.016311
17. Brewerton, T. D., Lafrance, A., & Mithoefer, M. C. (2021). The potential use of N-methyl-3,4-methylenedioxyamphetamine (MDMA) assisted psychotherapy in the treatment of eating disorders comorbid with PTSD. Medical Hypotheses, 146, 110367.
18. Brown, Brené, “Atlas of the Heart: MAPPING MEANINGFUL CONNECTION AND THE LANGUAGE OF HUMAN EXPERIENCE,” Random House, Nov. 30, 2021, ISBN 9780399592553
19. Busner, J., & Targum, S. D. (2007). The clinical global impressions scale: applying a research tool in clinical practice. Psychiatry (Edgemont, Pa. Township)), 4(7), 28-37.
20. Cadenhead, K. S., Geyer, M. A., & Braff, D. L. (1993). Impaired startle prepulse inhibition and habituation in patients with schizotypal personality disorder. American Journal of Psychiatry, 150, 1862-1862.
21. Campbell V. A., Gowran A. (2007). Alzheimer's disease; taking the edge off with cannabinoids? Br. J. Pharmacol. 152, 655-662. 10.1038/sj.bjp.0707446
22. Carhart-Harris, R. L., Bolstridge, M., Rucker, J., Day, C. M., Erritzoe, D., Kaelen, M., Bloomfield, M., Rickard, J. A., Forbes, B., Feilding, A., Taylor, D., Pilling, S., Curran, V. H., & Nutt, D. J. (2016). Psilocybin with psychological support for treatment-resistant depression: an open-label feasibility study. The lancet. Psychiatry, 3(7), 619-627. https://doi.org/10.1016/S2215-0366(16)30065-7
23. Carhart-Harris, R. L., Bolstridge, M., Day, C., Rucker, J., Watts, R., Erritzoe, D. E., Kaelen, M., Giribaldi, B., Bloomfield, M., Pilling, S., Rickard, J. A., Forbes, B., Feilding, A., Taylor, D., Curran, H. V., & Nutt, D. J. (2018). Psilocybin with psychological support for treatment-resistant depression: six-month follow-up. Psychopharmacology, 235(2), 399-408. https://doi.org/10.1007/s00213-01704771-x
24. Carvalho M, Pontes H, Remião F, Bastos M L, Carvalho F. Mechanisms underlying the hepatotoxic effects of ecstasy. Curr Pharm Biotechnol. 2010 August; 11(5):476-95. doi: 10.2174/138920110791591535. PMID: 20420570.
25. Castillo P. E., Younts T. J., Chavez A. E., Hashimotodani Y. (2012). Endocannabinoid signaling and synaptic function. Neuron 76, 70-81. 10.1016/j.neuron.2012.09.020
26. Chaliha, D., Mamo, J. C., Albrecht, M., Lam, V., Takechi, R., & Vaccarezza, M. (2021). A Systematic Review of the MDMA Model to Address Social Impairment in Autism. Current Neuropharmacology, Jan.
27. Chevaleyre V., Takahashi K. A., Castillo P. E. (2006). Endocannabinoid-mediated synaptic plasticity in the CNS. Annu. Rev. Neurosci. 29, 37-76. 10.1146/annurev.neuro.29.051605.112834
28. Cholden, L. S., Kurland, A., & Savage, C. (1955). Clinical reactions and tolerance to LSD in chronic schizophrenia. The Journal of nervous and mental disease, 122(3), 211-221.
29. Chong, JA., Fanger, C., Mandel-Brehm, J., Mcnamara, C., Moran, MM., Zhen, X. (Jul. 5, 2018) AU Pub. No. 2016/273866B2, “TRPA1 Inhibitors for Treating Pain”
30. Chu, A., &amp; Wadhwa, R. (2021, May 10). Selective serotonin reuptake inhibitors. StatPearls. Retrieved Oct. 26, 2021, from https://www.ncbi.nlm.nih.gov/books/NBK554406/.
31. Clegg, J. (2012). Stranger situations: Examining a self-regulatory model of socially awkward encounters. Group Processes and Intergroup Relations, 15 (6), 693-712 DOI: 10.1177/1368430212441637
32. Colman, A. M. (2015). “Dictionary of Psychology—Oxford Reference”. doi:10.1093/acref/9780199657681.001.0001
33. Dabell, J. (2019, November 25). Wallach-Kogan Creativity tests. John Dabell. Retrieved Dec. 7, 2021, from https://johndabell.com/2020/01/08/wallace-kogan-tests/.
34. Danforth, A. L., Grob, C. S., Struble, C., Feduccia, A. A., Walker, N., Jerome, L., Yazar-Klosinski, B., & Emerson, A. (2018). Reduction in social anxiety after MDMA-assisted psychotherapy with autistic adults: a randomized, double-blind, placebo-controlled pilot study. Psychopharmacology, 235(11), 3137-3148.
35. de la Torre, R., Farre, M., Ortuno, J., Mas, M., Brenneisen, R., Roset, P. N., Segura, J., & Cami, J. (2000). Non-linear pharmacokinetics of MDMA (‘ecstasy’) in humans. Br. J clinical pharmacology, 49(2), 104-109.
36. de la Torre, R., Yubero-Lahoz, S., Pardo-Lozano, R., & Farre, M. (2012). MDMA, methamphetamine, and CYP2D6 pharmacogenetics: what is clinically relevant?. Frontiers in genetics, 3, 235. https://doi.org/10.3389/fgene.2012.00235
37. Delgado-Peraza F., Ahn K. H., Nogueras-Ortiz C., Mungrue I. N., Mackie K., Kendall D. A., et al. (2016). Mechanisms of biased β-arrestin-mediated signaling downstream from the cannabinoid 1 receptor. Mol. Pharmacol. 89, 618-629. 10.1124/mol.115.103176
38. De Luca M A, Castelli M P, Loi B, Porcu A, Martorelli M, Miliano C, Kellett K, Davidson C, Stair J L, Schifano F, Di Chiara G. Native CB1 receptor affinity, intrinsic activity and accumbens shell dopamine stimulant properties of third generation SPICE/K2 cannabinoids: BB-22, 5F-PB-22, 5F-AKB-48 and STS-135. Neuropharmacology. 2016 June; 105:630-638.
39. Demuth D. G., Molleman A. (2006). Cannabinoid signalling. Life Sci. 78, 549-563. 10.1016/j.lfs. 2005.05.055
40. DiMarchi, RD., and Smiley, DL. (Aug. 4, 2020). U.S. Pat. No. 10,730,923B2, “Glucagon/GLP-1 receptor co-agonists”
41. Di Pilato, P., Niso, M., Adriani, W., Romano, E., Travaglini, D., Berardi, F., Colabufo, N., Perrone, R., Laviola, G., Lacivita, E. & Leopoldo, M. (2014). Selective agonists for serotonin 7 (5-HT7) receptor and their applications in preclinical models: an overview. Reviews in the Neurosciences, 25(3), 401-415. https://doi.org/10.1515/revneuro-2014-0009
42. Dittrich, A. (1998). The standardized psychometric assessment of altered states of consciousness (ASCs) in humans. Pharmacopsychiatry, 31(S 2), 80-84.
43. Dow-Edwards, D. (2018). Maternal drug abuse and adverse effects on neurobehavior of offspring. In Handbook of Developmental Neurotoxicology (pp. 487-497). Academic Press.
44. Dunlap, L. E., Andrews, A. M., & Olson, D. E. (2018). Dark Classics in Chemical Neuroscience: 3,4-Methylenedioxymethamphetamine. ACS Chemical Neuroscience. doi:10.1021/acschemneuro.8b001
45. Ecstasy could help the survivors of sexual assault (2012). MAPS. Archived at https://web.archive.org/save/https://maps.org/news/media/3356-ecstasy-cou Id-help-survivors-of-sexual-assault)
46. Edut, S., Rubovitch, V., Schreiber, S., & Pick, C. G. (2011). The intriguing effects of ecstasy (MDMA) on cognitive function in mice subjected to a minimal traumatic brain injury. Psychopharmacol, 214(4), 877-889
47. Eisner, B. (1989). Ecstasy: the MDMA story. Ronin Publishing (CA).
48. Eldeeb K., Leone-Kabler S., Howlett A. C. (2016). CB1 cannabinoid receptor-mediated increases in cyclic AMP accumulation are correlated with reduced Gi/o function. J. Basic Clin. Physiol. Pharmacol. 27, 311-322. 10.1515/jbcpp-2015-0096
49. Endogenous DMT and the Mental Entourage. Nov. 30, 2019. https://www.youtube.com/watch?v=OxrAUIIbkeM.
50. Ernsberger, P. The imidazoline receptor is a regulatory scaffold protein: implications in central regulation of the circulation. In: Ally A, Maher T J, Wyss J M, editors. Recent advances in cardiovascular research: from sleep to exercise; 2010. p. 141-153.
51. Feduccia, A. A., & Mithoefer, M. C. (2018). MDMA-assisted psychotherapy for PTSD: are memory reconsolidation and fear extinction underlying mechanisms?. Progress in neuro-psychopharmacology and biological psychiatry, 84, 221-228.
52. Fernández-Ruiz J., Moreno-Martet M., Rodriguez-Cueto C., Palomo-Garo C., Gomez-Canas M., Valdeolivas S., et al., (2011). Prospects for cannabinoid therapies in basal ganglia disorders. Br. J. Pharmacol. 163, 1365-1378. 10.1111/j.1476-5381.2011.01365.x
53. Fowler, Oleamide: a member of the endocannabinoid family?, Br. J. Pharmacol., 141(2):195-96 (2004)
54. Francois Petitet & Assunta Imperato (1998) The therapeutic applications of cannabinoid agonists and antagonists, Emerging Drugs, 3:1, 39-54, DOI: 10.1517/14728214.3.1.39
55. Frazer A, Hensler J G. Serotonin Receptors. In: Siegel G J, Agranoff B W, Albers R W, et al., editors. Basic Neurochemistry: Molecular, Cellular and Medical Aspects. 6th edition. Philadelphia: Lippincott-Raven; 1999. Available from: https://www.ncbi.nlm.nih.gov/books/NBK28234/
56. Freedman, A. M., Ebin, E. V., & Wilson, E. A. (1962). Autistic schizophrenic children: An experiment in the use of D-lysergic acid diethylamide (LSD-25). Archives of General Psychiatry, 6(3), 203-213.
57. Fride E., Perchuk A., Hall F. S., Uhl G. R., Onaivi E. S. (2006). Behavioral methods in cannabinoid research. Methods Mol. Med. 123, 269-290. 10.1385/1-59259-999-0:269
58. Frye, C. G., Wardle, M. C., Norman, G. J., & de Wit, H. (2014). MDMA decreases the effects of simulated social rejection. Pharmacology Biochemistry and Behavior, 117, 1-6.
59. Gant, T., and Sarshar, S. (Jul. 1, 2010). U.S. Pub. No. 2010/0166887A1, “DIBENZO[b,e][1,4]DIAZEPINE MODULATORS OF DOPAMINE RECEPTORS, SEROTONIN RECEPTORS, ADRENERGIC RECEPTORS, ACETYLCHOLINE RECEPTORS, AND/OR HISTAMINE RECEPTORS”
60. Gasser, P., Holstein, D., Michel, Y., Doblin, R., Yazar-Klosinski, B., Passie, T., & Brenneisen, R. (2014). Safety and efficacy of lysergic acid diethylamide-assisted psychotherapy for anxiety associated with life-threatening diseases. The Journal of nervous and mental disease, 202(7), 513.
61. Glass M., Felder C. C. (1997). Concurrent stimulation of cannabinoid CB1 and dopamine D2 receptors augments cAMP accumulation in striatal neurons: evidence for a Gs linkage to the CB1 receptor. J. Neurosci. 17, 5327-5333.
62. Glass M., Northup J. K. (1999). Agonist selective regulation of G proteins by cannabinoid CB1 and CB2 receptors. Mol. Pharmacol. 56, 1362-1369.
63. Glennon R A, Lee M, Rangisetty J B, Dukat M, Roth B L, Savage J E, McBride A, Rauser L, Hufeisen S, Lee D K. 2-Substituted tryptamines: agents with selectivity for 5-HT(6) serotonin receptors. J Med Chem. 2000 Mar. 9; 43(5):1011-8. doi: 10.1021/jm990550b. PMID: 10715164.
64. Gold, L. H., & Koob, G. F. (1989). MDMA produces stimulant-like conditioned locomotor activity. Psychopharmacology, 99(3), 352-356.
65. Goodman, D. A. (2003). MDMA in a severely disturbed man with psychosis, administered by his brother. MAPS. Archived at https://web.archive.org/web/20141205132212/https://maps.org/research-archive/mdma/edsstory.html
66. Greer, G., & Tolbert, R. (1986). Subjective reports of the effects of MDMA in a clinical setting. Journal of psychoactive drugs, 18(4), 319-327.
67. Greer, G. R., & Tolbert, R. (1998). A method of conducting therapeutic sessions with MDMA. Journal of psychoactive drugs, 30(4), 371-379.
68. Grof, S. (2008) LSD Psychotherapy (Ben Lomond, CA: Multidisciplinary Association for Psychedelic Studies.
69. Guo J., Ikeda S. R. (2004). Endocannabinoids modulate N-type calcium channels and G-protein-coupled inwardly rectifying potassium channels via CB1 cannabinoid receptors heterologously expressed in mammalian neurons. Mol. Pharmacol. 65, 665-674. 10.1124/mol.65.3.665
70. Hakim, M., Walia, H., Rafiq, M., Grannell, T., Cartabuke, R. S., & Tobias, J. D. (2016). Oxymetazoline Metered Dose Spray: Factors Affecting Delivery Volume. The journal of pediatric pharmacology and therapeutics: JPPT: the official journal of PPAG, 21(3), 247-251. https://doi.org/10.5863/1551-6776-21.3.247
71. Harman, W. W., McKim, R. H., Mogar, R. E., Faidman, J., and Stolaroff, M. J., (1966). Psychedelic Agents in Creative Problem-Solving: A pilot Study. Psychological Reports, 19(1), 211-227. doi:10.2466/pr0.1966.19.1.211
72. Harris, D. S., Baggott, M., Mendelson, J. H., Mendelson, J. E., & Jones, R. T. (2002). Subjective and hormonal effects of 3,4-methylenedioxymethamphetamine (MDMA) in humans. Psychopharmacology, 162(4), 396-405. doi:10.1007/s00213-002-1131-1
73. Harrison C, Traynor J R. The [35S]GTPgammaS binding assay: approaches and applications in pharmacology. Life Sci. 2003 Dec. 12; 74(4):489-508. doi: 10.1016/j.lfs.2003.07.005. PMID: 14609727.
74. Harvey, J. A. (2003). Role of the serotonin 5-HT2A receptor in learning. Learning & Memory, 10(5), 355-362
75. Hedlund P B, Sutcliffe J G (September 2004). “Functional, molecular and pharmacological advances in 5-HT7 receptor research”. Trends in Pharmacological Sciences. 25 (9): 481-6. doi:10.1016/j.tips.2004.07.002
76. Heifets B. D., Castillo P. E. (2009). Endocannabinoid signaling and long-term synaptic plasticity. Annu. Rev. Physiol. 71, 283-306. 10.1146/annurev.physiol.010908.163149
77. Heifets, B. D., & Malenka, R. C. (2016). MDMA as a probe and treatment for social behaviors. Cell, 166(2), 269-272.
78. Holland, J. 2000. Transcript of Lindesmith MDMA Seminar, 3/30/00 NYC. MAPS. Archived at https://web.archive.org/web/20141207133402/https://maps.org/research-archive/mdma/holland0300.html.
79. Holland. (2001). Using MDMA in the treatment of schizophrenia. Ecstasy: the complete guide. Park Street Press, Rochester.
80. Holland, Julie et al. (2001). Ecstasy: the complete guide; a comprehensive look at the risks and benefits of MDMA. Rochester, Vt., Park Street Press. ISBN 978-0-89281-857-0.
81. Hopkins A L, Groom C R (September 2002). “The druggable genome”. Nature Reviews. Drug Discovery. 1 (9): 727-30. doi:10.1038/nrd892
82. Howlett A. C. (2005). Cannabinoid receptor signaling. Handb. Exp. Pharmacol. 168, 53-79. 10.1007/3-540-26573-2_2
83. Howlett A. C., Barth F., Bonner T. I., Cabral G., Casellas P., Devane W. A., et al., (2002). International union of pharmacology. XXVII. Classification of cannabinoid receptors. Pharmacol. Rev. 54, 161-202. 10.1124/pr.54.2.161
84. Howlett A. C., Breivogel C. S., Childers S. R., Deadwyler S. A., Hampson R. E., Porrino L. J. (2004). Cannabinoid physiology and pharmacology: 30 years of progress. Neuropharmacology 47, 345-358. 10.1016/j.neuropharm.2004.07.030
85. Hoyer, D., Clarke, D. E., Fozard, J. R., Hartig, P. R., Martin, G. R., Mylecharane, E. J., Saxena, P. R., &amp; Humphrey, P. P. (1994, June 1). International Union of Pharmacology Classification of receptors for 5-hydroxytryptamine (serotonin). Pharmacological Reviews. Retrieved Oct. 27, 2021, from https://pharmrev.aspetjournals.org/content/46/2/157.abstract.
86. Hoyer D, Hannon J P, Martin G R (April 2002). “Molecular, pharmacological and functional diversity of 5-HT receptors”. Pharmacology Biochemistry and Behavior. 71 (4): 533-54. doi:10.1016/S0091-3057(01)00746-8
87. Hutcheson et al. (2011). Serotonin receptors and heart valve disease—it was meant 2B. Pharmacol. Therapeutics, 132(2), 146-57.
88. Hysek, C. M., G. Domes, and M. E. Liechti, MDMA enhances “mind reading” of positive emotions and impairs “mind reading” of negative emotions. Psychopharmacology (Berl), 2012. 222(2): p. 293-302.
89. Hysek, C. M., Schmid, Y., Simmler, L. D., Domes, G., Heinrichs, M., Eisenegger, C., . . . & Liechti, M. E. (2014). MDMA enhances emotional empathy and prosocial behavior. Social cognitive and affective neuroscience, 9(11), 1645-1652.
90. Ingrasci cited in Gertz, KR (1985) “HugDrug” alert: The agony of Ecstasy. Harper's Bazaar. November 1985 issue: 45-56.
91. Jakobuson, KA., Meru man, A., Wan B. JP Pat. No. 543147B2, “A3 adenosine receptor antagonist and A3 adenosine receptor partial agonist”
92. Jerome, L., Schuster, S., & Klosinski, B. B. (2013). Can MDMA play a role in the treatment of substance abuse?. Current drug abuse reviews, 6(1), 54-62.
93. Jerome, L., Feduccia, A. A., Wang, J. B., Hamilton, S., Yazar-Klosinski, B., Emerson, A., . . . & Doblin, R. (2020). Long-term follow-up outcomes of MDMA-assisted psychotherapy for treatment of PTSD: a longitudinal pooled analysis of six phase 2 trials. Psychopharmacology, 237, 2485-2497.
94. Jin W., Brown S., Roche J. P., Hsieh C., Celver J. P., Kovoor A., et al., (1999). Distinct domains of the CB1 cannabinoid receptor mediate desensitization and internalization. J. Neurosci. 19, 3773-3780.
95. Johansen PØ, Krebs T S. (2009). How could MDMA (ecstasy) help anxiety disorders? A neurobiological rationale. Journal of Psychopharmacology. June; 23(4):389-91.
96. Johnson, M. W., Garcia-Romeu, A., Cosimano, M. P., & Griffiths, R. R. (2014). Pilot study of the 5-HT2AR agonist psilocybin in the treatment of tobacco addiction. Journal of psychopharmacology, 28(11), 983-992.
97. Johnson, M. W., Richards, W. A., & Griffiths, R. R. (2008). Human hallucinogen research: guidelines for safety. Journal of psychopharmacology, 22(6), 603-620.
98. Kalant H. (2001). The pharmacology and toxicology of “ecstasy” (MDMA) and related drugs. CMAJ: Canadian Medical Association journal=journal de l'Association medicale canadienne, 165(7), 917-928.
99. Kamboj, S. K., Kilford, E. J., Minchin, S., Moss, A., Lawn, W., Das, R. K., . . . & Freeman, T. P. (2015). Recreational 3,4-methylenedioxy-N-methylamphetamine (MDMA) or ‘ecstasy’ and self-focused compassion: preliminary steps in the development of a therapeutic psychopharmacology of contemplative practices. Journal of Psychopharmacology, 29(9), 961-970.
100. Kamilar-Britt, P., & Bedi, G. (2015). The prosocial effects of 3,4-methylenedioxymethamphetamine (MDMA): controlled studies in humans and laboratory animals. Neuroscience & Biobehavioral Reviews, 57, 433-446
101. Kano M., Ohno-Shosaku T., Hashimotodani Y., Uchigashima M., Watanabe M. (2009). Endocannabinoid-mediated control of synaptic transmission. Physiol. Rev. 89, 309-380. 10.1152/physrev.00019.2008
102. Kaplunov, E. (2021, April 30). How do we measure creativity? Psychreg. Retrieved Nov. 18, 2021, from https://www.psychreg.org/how-do-we-measure-creativity/.
103. Kario, K. (2018). “Central Sympathetic Agents and Direct Vasodilators.” Hypertension: A Companion to Braunwald's Heart Disease. (3): 254-260. doi.org/10.1016/B978-0-323-42973-3.00026-3.
104. Katona I., Freund T. F. (2012). Multiple functions of endocannabinoid signaling in the brain. Annu. Rev. Neurosci. 35, 529-558. 10.1146/annurev-neuro-062111-150420
105. Kelly A Berg, William P Clarke, Making Sense of Pharmacology: Inverse Agonism and Functional Selectivity, International Journal of Neuropsychopharmacology, Volume 21, Issue 10, October 2018, Pages 962-977, https://doi.org/10.1093/ijnp/pyy071
106. Kendall, D. A., and Yudowski G. A. (2017). “Cannabinoid Receptors in the Central Nervous System: Their Signaling and Roles in Disease.” Frontiers in Cellular Neuroscience. (10): 294. DOI=10.3389/fncel.2016.00294
107. Klein, L. M., Cozzi, N. V., Daley, P. F., Brandt, S. D., & Halberstadt, A. L. (2018). Receptor binding profiles and behavioral pharmacology of ring-substituted N,N-diallyltryptamine analogs. Neuropharmacology, 142, 231-239. https://doi.org/10.1016/j.neuropharm.2018.02.028
108. Klein T. W. (2005). Cannabinoid-based drugs as anti-inflammatory therapeutics. Nat. Rev. Immunol. 5, 400-411. 10.1038/nri1602
109. Kristensen L. J., Jensen A. A., Marcher-Rorsted E., and Leth-Petersen S. (May 13, 2021). U.S. Pub. No. 2021/0137908A1, “5-ht2a agonists for use in treatment of depression”
110. Laprairie R. B., Bagher A. M., Kelly M. E. M., Denovan-Wright E. M. (2016). Biased type 1 cannabinoid receptor signaling influences neuronal viability in a cell culture model of Huntington disease. Mol. Pharmacol. 89, 364-375. 10.1124/mol.115.101980
111. Laprairie R. B., Bagher A. M., Kelly M. E. M., Dupre D. J., Denovan-Wright E. M. (2014). Type 1 cannabinoid receptor ligands display functional selectivity in a cell culture model of striatal medium spiny projection neurons. J. Biol. Chem. 289, 24845-24862. 10.1074/jbc.m114.557025
112. Lau, W. M. (2008). “Scope and Limitations of the Co-Drug Approach to Topical Drug Delivery”. Current Pharmaceutical Design. 14 (8): 794-802. doi:10.2174/138161208784007653
113. Le, J. (2021, November 17). Drug administration—drugs. Merck Manuals Consumer Version. Retrieved Nov. 28, 2021, from https://www.merckmanuals.com/home/drugs/administration-and-kinetics-of-drugs/drug-administration.
114. Lees P, Cunningham F M, Elliott J (December 2004). “Principles of pharmacodynamics and their applications in veterinary pharmacology”. Journal of Veterinary Pharmacology and Therapeutics. 27 (6): 397-414. doi:10.1111/j.1365-2885.2004.00620.x
115. Maguire J J, Kuc R E, Davenport A P. Radioligand binding assays and their analysis. Methods Mol Biol. 2012; 897:31-77. doi: 10.1007/978-1-61779-909-9_3. PMID: 22674160.
116. Marcello Leopoldo, Enza Lacivita, Marialessandra Contino, Nicola A. Colabufo, Francesco Berardi, and Roberto Perrone, Structure—Activity Relationship Study on N-(1,2,3,4-Tetrahydronaphthalen-1-yl)-4-aryl-1-piperazinehexanamides, a Class of 5-HT7 Receptor Agents, Journal of Medicinal Chemistry 2007 50 (17), 4214-4221, DOI: 10.1021/jm070487n
117. Mithoefer, M. (2015) A Manual for MDMA-Assisted Psychotherapy in the Treatment of Posttraumatic Stress Disorder. Version 7. Multidisciplinary association for Psychedelic Studies. Retrieved from https://maps.org/research-archive/mdma/MDMA-Assisted-Psychotherapy-Treatment-Manual-Version7-19Aug15-FINAL.pdf
118. Leopoldo, M., Lacivita, E., Berardi, F., Perrone, R., & Hedlund, P. B. (2011). Serotonin 5-HT7 receptor agents: Structure-activity relationships and potential therapeutic applications in central nervous system disorders. Pharmacology & therapeutics, 129(2), 120-148. https://doi.org/10.1016/j.pharmthera.2010.08.013
119. Leterrier C., Bonnard D., Carrel D., Rossier J., Lenkei Z. (2004). Constitutive endocytic cycle of the CB1 cannabinoid receptor. J. Biol. Chem. 279, 36013-36021. 10.1074/jbc.M403990200
120. Li G, Regunathan S, Barrow C J, Eshraghi J, Cooper R, Reis D J, Agmatine: an endogenous clonidine-displacing substance in the brain. Science. 1994 Feb. 18; 263(5149):966-9.
121. Liechti, M. E., Geyer, M. A., Hell, D., & Vollenweider, F. X. (2001). Effects of MDMA (ecstasy) on prepulse inhibition and habituation of startle in humans after pretreatment with citalopram, haloperidol, or ketanserin. Neuropsychopharmacology, 24(3), 240-252.
122. Lin, H. Q., Burden, P. M., Christie, M. J., & Johnston, G. A. (1999). The anxiogenic-like and anxiolytic-like effects of MDMA on mice in the elevated plus-maze: a comparison with amphetamine. Pharmacology Biochemistry and Behavior, 62(3), 403-408.
123. “Lisa”. (n.d.) MDMA (At a Rave) Helps Heal Trauma from Sexual Assault. MAPS. Archived at https://web.archive.org/web/20141205132127/https://maps.org/research-archive/mdma/mdmalisa.html).
124. Little P. J., Compton D. R., Johnson M. R., Melvin L. S., Martin B. R. (1988). Pharmacology and stereoselectivity of structurally novel cannabinoids in mice. J. Pharmacol. Exp. Ther. 247, 1046-1051.
125. Mackie K., Lai Y., Westenbroek R., Mitchell R. (1995). Cannabinoids activate an inwardly rectifying potassium conductance and inhibit Q-type calcium currents in AtT20 cells transfected with rat brain cannabinoid receptor. J. Neurosci. 15, 6552-6561.
126. Maroso M., Szabo G. G., Kim H. K., Alexander A., Bui A. D., Lee S.-H., et al., (2016). Cannabinoid control of learning and memory through HCN channels. Neuron 89, 1059-1073. 10.1016/j.neuron.2016.01.023
127. Marsicano G., Lutz B. (2006). Neuromodulatory functions of the endocannabinoid system. J. Endocrinol. Invest. 29, 27-46.
128. Martini L., Waldhoer M., Pusch M., Kharazia V., Fong J., Lee J. H., et al., (2007). Ligand-induced down-regulation of the cannabinoid 1 receptor is mediated by the G-protein-coupled receptor-associated sorting protein GASP1. FASEB J. 21, 802-811. 10.1096/fj.06-7132com
129. Mason, N. L., Kuypers, K. P. C., Reckweg, J. T. et al. Spontaneous and deliberate creative cognition during and after psilocybin exposure. Transl Psychiatry 11, 209 (2021). https://doi.org/10.1038/s41398-C21-01335-5
130. Mattson, M. P., Maudsley, S., & Martin, B. (2004). BDNF and 5-HT: a dynamic duo in age-related neuronal plasticity and neurodegenerative disorders. Trends in neurosciences, 27(10), 589-594.
131. Mayo Foundation for Medical Education and Research. (2019, November 27). What you can expect from medical marijuana. Mayo Clinic. Retrieved Oct. 25, 2021, from www.mayoclinic.org/healthy-lifestyle/consumer-health/in-depth/medical-marijuana/art-20137855.
132. Mayo Foundation for Medical Education and Research. (2019, November 9). Schizoaffective disorder. Mayo Clinic. Retrieved Oct. 29, 2021, from https://www.mayoclinic.orci/diseases-conditions/schizoaffective-disorder/symptoms-causes/syc-20354504.
133. McDonough, S. I. (2013). Calcium ion channels: challenges and successes in drug discovery. WIREs Membr Transp Signal2013, 2:85-104. doi: 10.1002/wmts.71
134. McElroy, S. L. (2004). Diagnosing and treating comorbid (complicated) bipolar disorder. The Journal of clinical psychiatry, 65(suppl 15), 35-44.
135. MDMA and post-traumatic stress disorder. (n.d.) MAPS. Retrieved from https://maps.org/research-archive/mdma/ptsdNG.html.
136. Mechoulam R., Parker L. A. (2013). The endocannabinoid system and the brain. Annu. Rev. Psychol. 64, 21-47. 10.1146/annurev-psych-113011-143739
137. Mitchell, J. M., Bogenschutz, M., Lilienstein, A. et al. MDMA-assisted therapy for severe PTSD: a randomized, double-blind, placebo-controlled phase 3 study. Nat Med 27, 1025-1033 (2021). https://doi.org/10.1038/s41591-C21-01336-3
138. Mithoefer, M. (2013). MDMA-assisted psychotherapy: How different is it from other psychotherapy. Manifesting minds: A review of psychedelics in science, medicine, sex, and spirituality, 125.
139. Mithoefer, M. C., Grob, C. S., & Brewerton, T. D. (2016). Novel psychopharmacological therapies for psychiatric disorders: psilocybin and MDMA. The Lancet Psychiatry, 3(5), 481-488
140. Moonzwe, L. S., Schensul, J. J., & Kostick, K. M. (2011). The role of MDMA (Ecstasy) in coping with negative life situations among urban young adults. Journal of psychoactive drugs, 43(3), 199-210.
141. Moraczewski, J., &amp; Aedma, K. (2020, December 7). Tricyclic antidepressants. StatPearls [Internet]. Retrieved Oct. 26, 2021, from https://www.ncbi.nlm.nih.gov/books/NBK557791/.
142. Müller, F., Holze, F., Dolder, P. et al. MDMA-induced changes in within-network connectivity contradict the specificity of these alterations for the effects of serotonergic hallucinogens. Neuropsychopharmacol. 46, 545-553 (2021).
143. Mulukom, V., Patterson, R. E., & van Elk, M. (2020). Broadening Your Mind to Include Others: The relationship between serotonergic psychedelic experiences and maladaptive narcissism. Psychopharmacology, 237(9), 2725-2737.
144. Nardou, Romain, et al. “Oxytocin-dependent reopening of a social reward learning critical period with MDMA.” Nature 569.7754 (2019): 116-120.
145. Nature Publishing Group. (2005). GPCR. Nature news. Retrieved Oct. 25, 2021, from https://www.nature.com/scitable/topicpage/gper-14047471/.
146. Nichols, D. E., & Oberlender, R. (1990). Structure-activity relationships of MDMA and related compounds: a new class of psychoactive drugs?. Annals of the New York Academy of Sciences, 600, 613-625. https://doi.org/10.1007/978-1-4613-1485-1_7
147. NIDA. 2021, April 13. What are the effects of MDMA?. Retrieved from https://www.drugabuse.gov/publications/research-reports/mdma-ecstasy-abuse/what-are-effects-mdma on 2021, October 29.
148. n.d. MDMA and post-traumatic stress disorder. MAPS. Archived at https://web.archive.org/web/20141205132128/https://maps.org/research-archive/mdma/ptsdNG.html
149. Nov. 5, 2020, Lecture: “Mechanisms of Consciousness & Entourage Collapse in Mental Disorders”, Psychedelic Science Interest Group, Yale.
150. Nguyen P. T., Schmid C. L., Raehal K. M., Selley D. E., Bohn L. M., Sim-Selley L. J. (2012). β-arrestin2 regulates cannabinoid CB1 receptor signaling and adaptation in a central nervous system region-dependent manner. Biol. Psychiatry 71, 714-724. 10.1016/j.biopsych.2011.11.027
151. Nichols, D., and Yu, B. (May 9, 2015). U.S. Pat. No. 9,642,819 B2 “Low dosage serotonin 5-HT2A receptor agonist to suppress inflammation”
152. Nikolic, K., & Agbaba, D. (2012). Imidazoline antihypertensive drugs: selective i(1)-imidazoline receptors activation. Cardiovascular therapeutics, 30(4), 209-216. https://doi.org/10.1111/j.1755-5922.2011.00269.x
153. Oeri H. E. (2020). Beyond ecstasy: Alternative entactogens to 3,4-methylenedioxymethamphetamine with potential applications in psychotherapy. Journal of psychopharmacology (Oxford, England), 35(5), 512-536. https://doi.org/10.1177/0269881120920420
154. Oehen, P., Traber, R., Widmer, V., & Schnyder, U. (2013). A randomized, controlled pilot study of MDMA (±3,4-Methylenedioxymethamphetamine)-assisted psychotherapy for treatment of resistant, chronic Post-Traumatic Stress Disorder (PTSD). Journal of psychopharmacology, 27(1), 40-52.
155. Passie, T. (2012) Healing with Entactogens: Therapist and Patient Perspectives on MDMA-Assisted Group Psychotherapy (Ben Lomond, CA: Multidisciplinary Association for Psychedelic Studies.
156. Passie, T., Hartmann, U., Schneider, U., Emrich, H. M., & Krüger, T. H. (2005). Ecstasy (MDMA) mimics the post-orgasmic state: impairment of sexual drive and function during acute MDMA-effects may be due to increased prolactin secretion. Medical hypotheses, 64(5), 899-903.
157. Patel, R., & Titheradge, D. (2015). MDMA for the treatment of mood disorder: all talk no substance?. Therapeutic advances in psychopharmacology, 5(3), 179-188.
158. Pauwels, D., and Andersen, T. (Aug. 29, 2019). U.S. Pub. No. 2019/0264385 A1, “Methods for preparing cannabis hurd fiber, purified cannabis hurd fiber, and articles containing the purified cannabis hurd fiber”
159. Pelé-Tounian, A., Wang, X., Rondu, F., Lamouri, A., Touboul, E., Marc, S., Dokhan, R., Pfeiffer, B., Manechez, D., Renard, P., Guardiola-Lemaitre, B., Godfroid, J. J., Penicaud, L., & Ktorza, A. (1998). Potent antihyperglycaemic property of a new imidazoline derivative S-22068 (PMS 847) in a rat model of NIDDM. British journal of pharmacology, 124(8), 1591-1596. https://doi.org/10.1038/sj.bjp.0701989
160. Piletz, J. E., Wang, G., Zhu H. Cell signaling by imidazoline-1 receptor candidate, IRAS, and the nischarin homologue. Ann N Y Acad Sci, 1009 (2003), pp. 392-399
161. Pertwee R. G., Howlett A. C., Abood M. E., Alexander S. P. H., Di Marzo V., Elphick M. R., et al., (2010). International union of basic and clinical pharmacology. LXXIX. Cannabinoid receptors and their ligands: beyond CB1 and CB2. Pharmacol. Rev. 62, 588-631. 10.1124/pr.110.003004
162. Pietrangelo, A. (2021, September 9). Types of depression: Chronic, episodes, and more. Healthline. Retrieved Oct. 26, 2021, from https://www.healthline.com/health/types-of-depression.
163. Preller, K. H., & Vollenweider, F. X. (2019). Modulation of social cognition via hallucinogens and “entactogens”. Frontiers in psychiatry, 10, 881.
164. Preskey, N. (2018). MDMA Could Help Treat Eating Disorders. Vice.com. Archived at: web.archive.org/web/20210320162753/https://www.vice.com/en/article/9kzy87/mdma-treatment-eating-disorders
165. Pryce G., Ahmed Z., Hankey D. J. R., Jackson S. J., Croxford J. L., Pocock J. M., et al., (2003). Cannabinoids inhibit neurodegeneration in models of multiple sclerosis. Brain 126, 2191-2202. 10.1093/brain/awg224
166. Psychedelic therapy for Tourette's, (2018). Reddit. Archived at https://web.archive.org/web/20210321105532/https://www.reddit.com/r/Tourettes/comments/78rfan/psycadelic_therapy_for_tourettes/Ramaekers, J. G., & Kuypers, K. P. (2006). Acute effects of 3,4-methylenedioxymethamphetamine (MDMA) on behavioral measures of impulsivity: alone and in combination with alcohol. Neuropsychopharmacology, 31(5), 1048-1055.
167. Radwan, M. M., Chandra, S., Gul, S., & ElSohly, M. A. (2021). Cannabinoids, Phenolics, Terpenes and Alkaloids of Cannabis. Molecules (Basel, Switzerland), 26(9), 2774. https://doi.org/10.3390/molecules26092774
168. Ramaekers, J. G., & Kuypers, K. P. (2006). Acute effects of 3,4-methylenedioxymethamphetamine (MDMA) on behavioral measures of impulsivity: alone and in combination with alcohol. Neuropsychopharmacology, 31(5), 1048-1055
169. Randall, S., Johanson, C. E., Tancer, M., & Roehrs, T. (2009). Effects of acute 3,4-methylenedioxymethamphetamine on sleep and daytime sleepiness in MDMA users: a preliminary study. Sleep, 32(11), 1513-1519.
170. Ray, T. S. (2010). Psychedelics and the Human Receptorome. PLoS ONE. http://dx.plos.org/10.1371/journal.pone.0009019.
171. Ray, T. S. (2012). Mental Organs and the Origins of Mind. In L. Swan (Ed) Origins of Mind, pp. 301-326. New York/Heidelberg: Springer. http://life.ou.edu/pubs/OriginsOfMindTeachingpdf.
172. Ray, T. S. (2015). Constructing the ecstasy of MDMA from its component mental organs: Proposing the primer/probe method. Medical Hypotheses. http://www.medical-hypotheses.com/article/S0306-9877(15)00472-7/fulltext.
173. Ray T. S. (2016). Constructing the ecstasy of MDMA from its component mental organs: Proposing the primer/probe method. Medical hypotheses, 87, 48-60. https://doi.org/10.1016/j.mehy.2015.12.018
174. Ray, T. S. (2017). Mental Organs and the Breadth and Depth of Consciousness. Transform Press. Jun. 27, 2017. Video, PowerPoint, & Manuscript. https://www.transformpress.com/breadth-and-depth.
175. Ray, T. S. (2017). Mental Organs And The Breadth And Depth Of Consciousness. Breaking Convention. Published on Aug. 19, 2017. Video and abstract. https://www.youtube.com/watch?v=6fMLCKBAosE.
176. Ray, T. S. (2019). Endogenous DMT and the Mental Entourage. https://www.youtube.com/watch?v=OxrAUIIbkeM
177. Ray, T. S. (Jun. 24, 2009), PCT Pub. No. WO2010/151258A1, “Methods of treating psychological conditions”
178. Ray, T. S. (2020). Sex in the Time of Plague: Awakening Melatonin into the Entourage. September 2020. https://eagle.sbs.arizona.edu/sc/report_poster_detail.php?abs=3967
179. Regunathan, S; Reis, D J (April 1996). “Imidazoline Receptors and Their Endogenous Ligands”. Annual Review of Pharmacology and Toxicology. 36 (1): 511-544. doi:10.1146/annurev.pa.36.040196.002455. PMID 8725400.
180. Reiff, C., Richman, E., Nemeroff, C., Carpenter, L., Widge, A., Rodriguez, C., . . . McDonald, W. (2020). Psychedelics and psychedelic-assisted psychotherapy. American Journal of Psychiatry, 177(5), 391-410. doi:10.1176/appi.ajp.2019.19010035
181. Rickli, A., Moning, O. D., Hoener, M. C., &amp; Liechti, M. E. (2016). Receptor interaction profiles of novel psychoactive tryptamines compared with classic hallucinogens. European Neuropsychopharmacology, 26(8), 1327-1337. https://doi.org/10.1016/j.euroneuro.2016.05.001
182. Roberts, C. A., Quednow, B. B., Montgomery, C., & Parrott, A. C. (2018). MDMA and brain activity during neurocognitive performance: An overview of neuroimaging studies with abstinent ‘Ecstasy’ users. Neuroscience & Biobehavioral Reviews, 84, 470-482.
183. Roche J. P., Bounds S., Brown S., Mackie K. (1999). A mutation in the second transmembrane region of the CB1 receptor selectively disrupts G protein signaling and prevents receptor internalization. Mol. Pharmacol. 56, 611-618. 10.1124/mol.56.3.611
184. Roth B. L., (2018). National Institute of Mental Health Psychoactive Drug Screening Program (NIMH PDSP) Assay Protocol Book Version III, Dept. Pharmacology, University of North Carolina at Chapel Hill. Retrieved from: pdsp.unc.edu/pdspweb/content/UNC-CH%20Protocol%20Book.pdf
185. Rozenfeld R. (2011). Type I cannabinoid receptor trafficking: all roads lead to lysosome. Traffic 12, 12-18.
186. Russo, Taming THC: potential cannabis synergy and phytocannabinoid-terpenoid entourage effects, British J. of Pharm., 163:1344-64 (2011)
187. Schäfer U, Burgdorf C, Engelhardt A, Kurz T, Richardt G. Presynaptic effects of moxonidine in isolated buffer perfused rat hearts: role of imidazoline-1 receptors and alpha2-adrenoceptors. J Pharmacol Exp Ther. 2002 December; 303(3):1163-70. doi: 10.1124/jpet.102.041657. PMID: 12438540.
188. Schmid, Y., Gasser, P., Oehen, P., & Liechti, M. E. (2020). Acute subjective effects in LSD- and MDMA-assisted psychotherapy. Journal of Psychopharmacology, 0269881120959604.
189. Schmid, Y., Hysek, C. M., Simmler, L. D., Crockett, M. J., Quednow, B. B., & Liechti, M. E. (2014). Differential effects of MDMA and methylphenidate on social cognition. Journal of psychopharmacology, 28(9), 847-856.
190. Schultz, P., Lairson, L., Deshmukh, V., Lyssiotis C. (May 18, 2017). U.S. Pat. No. 10,493,080, “Directed differentiation of oligodendrocyte precursor cells to a myelinating cell fate”
191. Scotter E. L., Abood M. E., Glass M. (2010). The endocannabinoid system as a target for the treatment of neurodegenerative disease. Br. J. Pharmacol. 160, 480-498. 10.1111/j.1476-5381.2010.00735.x
192. Selvey, L., Carlos, M., Maffuid, P., Shan, Y., Betts, W., Given, D., Hard, R., Shao, Zezhi., (Mar. 10, 2020). U.S. Pat. No. 10,583,122 B2, “Composition of a 5-HT2A serotonin receptor modulator useful for the treatment of disorders related thereto”
193. Sessa, B. (2010). Self-medication of LSD and MDMA to treat mental disorders: A case series. The Journal of Alternative Medicine Research, 2(2), 245-249.
194. Sessa, B. (2018). Why MDMA therapy for Alcohol Use Disorder? and why now? Neuropharmacology, 142, 83-88. https://doi.org/10.1016/j.neuropharm.2017.11.004
195. Sessa, B., & Fischer, F. M. (2015). Underground MDMA-, LSD- and 2-CB-assisted individual and group psychotherapy in Zurich: Outcomes, implications and commentary. Drug Science, Policy and Law, 2, 2050324515578080.
196. Sevelius, J. (2019). Psychedelic-assisted therapy with transgender and gender diverse individuals. MAPS Bulletin, 29(1).
197. Sex in the Time of Plague: Awakening Melatonin into the Entourage. September 2020. https://eagle.sbs.arizona.edu/sc/report_poster_detail.php?abs=3967.
198. Shannon, Colbert, & Hughes (2021). Therapeutic and Social Uses of MDMA, in Grob & Grigsby. Handbook of Medical Hallucinogens, Guilford Press, NY).
199. Shortle, M. (2016, Sep. 12). Your brain on cognitive creativity—with illustrations. Grio Blog. Retrieved Nov. 18, 2021, from https://blog.grio.com/2016/09/your-brain-on-cognitive-creativity-with-illustrations.html.
200. Shulgin, A. & Shulgin, A. (1991). PiHKAL: A chemical love story. Transform Press. Berkeley, CA.
201. Silva et al., Influences of phytocannabinoids on drug-drug interactions and their clinical implications, doi.org/10.1016/j.pharmthera.2020.107621 (2020)
202. Socially awkward. Symptoms and facts. King University Online. (2021). Retrieved Nov. 18, 2021, from https://online.king.edu/news/socially-awkward-symptoms/.
203. Soltesz I., Alger B. E., Kano M., Lee S.-H., Lovinger D. M., Ohno-Shosaku T., et al., (2015). Weeding out bad waves: towards selective cannabinoid circuit control in epilepsy. Nat. Rev. Neurosci. 16, 264-277. 10.1038/nrn3937
204. Sotnikova, T. D., Beaulieu, J. M., Barak, L. S., Wetsel, W. C., Caron, M. G., & Gainetdinov, R. R. (2005). Dopamine-independent locomotor actions of amphetamines in a novel acute mouse model of Parkinson disease. PLoS Biol, 3(8), e271.
205. Stahl, S. M. (2013). Stahl's Essential Psychopharmacology. Neuroscientific Basis and Practical Applications (4.): Cambridge University Press.
206. Straiker A., Mackie K. (2005). Depolarization-induced suppression of excitation in murine autaptic hippocampal neurones. J. Physiol. 569, 501-517. 10.1113/jphysiol.2005.091918
207. Strange P. G. (2008). Agonist binding, agonist affinity and agonist efficacy at G protein-coupled receptors. British journal of pharmacology, 153(7), 1353-1363. https://doi.org/10.1038/sj.bjp.0707672
208. Swinney D C (September 2004). “Biochemical mechanisms of drug action: what does it take for success?”. Nature Reviews. Drug Discovery. 3 (9): 801-8. doi:10.1038/nrd1500
209. Tai, S., & Fantegrossi, W. E. (2014). Synthetic Cannabinoids: Pharmacology, Behavioral Effects, and Abuse Potential. Current addiction reports, 1(2), 129-136. doi.org/10.1007/s40429-014-0014-y
210. Tai, S. J., Nielson, E. M., Lennard-Jones, M., Johanna Ajantaival, R. L., Winzer, R., Richards, W. A., Reinholdt, F., Richards, B. D., Gasser, P., & Malievskaia, E. (2021). Development and Evaluation of a Therapist Training Program for Psilocybin Therapy for Treatment-Resistant Depression in Clinical Research. Frontiers in psychiatry, 12, 586682. https://doi.org/10.3389/fpsyt.2021.586682
211. Torres, F. (n.d.). What is posttraumatic stress disorder? What Is PTSD? Retrieved Oct. 26, 2021, from https://www.psychiatry.org/patients-families/ptsd/what-is-ptsd.
212. Touriño, C., Maldonado, R., & Valverde, O. (2007). MDMA attenuates THC withdrawal syndrome in mice. Psychopharmacology, 193(1), 75-84.
213. Tucker, G. T., Lennard, M. S., Ellis, S. W., Woods, H. F., Cho, A. K., Lin, L. Y., Hiratsuka, A., Schmitz, D. A. and Chu, T. Y. Y., 1994. The demethylenation of methylenedioxymethamphetamine (“ecstasy”) by debrisoquine hydroxylase (CYP2D6). Biochemical pharmacology, 47(7), pp. 1151-1156
214. Twitchell W., Brown S., Mackie K. (1997). Cannabinoids inhibit N- and P/Q-type calcium channels in cultured rat hippocampal neurons. J. Neurophysiol. 78, 43-50.
215. U.S. National Library of Medicine. (2020, June 18). Depression: How effective are antidepressants? InformedHealth.org. Retrieved Oct. 26, 2021, from https://www.ncbi.nlm.nih.gov/books/NBK361016/.
216. Van Der Kolk, Bessel (Aug. 24, 2021). Transcription of “The Ezra Klein Show,” available at www.nytimes.com/2021/08/24/opinion/ezra-klein-podcast-van-der-kolk.html?showTranscript=1
217. Varga E. V., Georgieva T., Tumati S., Alves I., Salamon Z., Tollin G., et al. (2008). Functional selectivity in cannabinoid signaling. Curr. Mol. Pharmacol. 1, 273-284. 10.2174/1874-470210801030273
218. Varvel S. A., Martin B. R., Lichtman A. H. (2007). Lack of behavioral sensitization after repeated exposure to THC in mice and comparison to methamphetamine. Psychopharmacology (Berl) 193, 511-519.
219. Vauquelin G, Van Liefde I, Birzbier B B, Vanderheyden P M (August 2002). “New insights in insurmountable antagonism”. Fundamental & Clinical Pharmacology. 16 (4): 263-72. doi: 10.1046/j.1472-8206.2002.00095.x
220. Wagner, M. T., Mithoefer, M. C., Mithoefer, A. T., MacAulay, R. K., Jerome, L., Yazar-Klosinski, B., & Doblin, R. (2017). Therapeutic effect of increased openness: Investigating mechanism of action in MDMA-assisted psychotherapy. Journal of Psychopharmacology, 31(8), 967-974. https://doi.org/10.1177/0269881117711712
221. Wilder, D. (2018). Using MDMA to treat eating disorders. Psychedelic Times. Archived at: web. archive.org/web/20200930091405/https://psychedelictimes.com/using-mdma-treat-eating-disorders/
222. Wilson R. I., Nicoll R. A. (2001). Endogenous cannabinoids mediate retrograde signalling at hippocampal synapses. Nature 410, 588-592. 10.1038/35069076
223. Wolfson cited in Passie, T. (2018). The early use of MDMA (‘Ecstasy’) in psychotherapy (1977-1985). Drug Science, Policy and Law, 4, 2050324518767444.
224. Wolfson, P. E., Andries, J., Feduccia, A. A., Jerome, L., Wang, J. B., Williams, E., . . . & Doblin, R. (2020). MDMA-assisted psychotherapy for treatment of anxiety and other psychological distress related to life-threatening illnesses: a randomized pilot study. Scientific reports, 10(1), 1-15.
225. Wu D.-F., Yang L.-Q., Goschke A., Stumm R., Brandenburg L.-O., Liang Y.-J., et al., (2008). Role of receptor internalization in the agonist-induced desensitization of cannabinoid type 1 receptors. J. Neurochem. 104, 1132-1143. 10.1111/j.1471-4159.2007.05063.x
226. Wunderli, M. D., Vonmoos, M., Treichler, L., Zeller, C., Dziobek, I., Kraemer, T., . . . & Quednow, B. B. (2018). Social cognition and interaction in chronic users of 3,4-methylenedioxymethamphetamine (MDMA, “ecstasy”). International Journal of Neuropsychopharmacology, 21(4), 333-344.
227. Yartsev, A. (2021). Full agonists, partial agonists and inverse agonists. Retrieved Nov. 9, 2021, from https://derangedphysiology.com/main/cicm-primary-exam/required-reading/pharmacodynamics/Chapter%20417/full-agonists-partial-agonists-and-inverse-agonists.v
228. Yazar-Klosinski, B. B., & Mithoefer, M. C. (2017). Potential psychiatric uses for MDMA. Clinical pharmacology & therapeutics, 101(2), 194-196.
229. Young, M. B., Andero, R., Ressler, K. J., & Howell, L. L. (2015). MDMA facilitates fear extinction learning. Translational psychiatry, 5(9), e634-e634.
230. Zheng C, Chen L, Chen X, He X, Yang J, et al. (2013). “The Second Intracellular Loop of the Human Cannabinoid CB2 Receptor Governs G Protein Coupling in Coordination with the Carboxyl Terminal Domain.” PLOS ONE 8(5): e63262. https://doi.org/10.1371/journal.pone.0063262
231. Zemishlany, Z., Aizenberg, D., & Weizman, A. (2001). Subjective effects of MDMA (‘Ecstasy’) on human sexual function. European Psychiatry, 16(2), 127-130.
232. Ziegler D, Haxhiu M A, Kaan E C, Papp J G, Ernsberger P. Pharmacology of moxonidine, an I1-imidazoline receptor agonist. J Cardiovasc Pharmacol. 1996; 27 Suppl 3:S26-37. doi: 10.1097/00005344-199627003-00005.
233. Zimmer A., Zimmer A. M., Hohmann A. G., Herkenham M., Bonner T. I. (1999). Increased mortality, hypoactivity and hypoalgesia in cannabinoid CB1 receptor knockout mice. Proc. Natl. Acad. Sci. USA 96, 5780-5785.
234. “Zoe”. (2014). Bulimarexia and MDMA—my experience. MAPS. Archived at https://web.archive.org/web/20141207203134/https://maps.org/research-archive/mdma/healing/buli.html).

The invention claimed is:

1. A therapeutic combination for eliciting an entactogenic mindstate, comprising an imidazoline-1 (I₁) agent and a 5-HT₇ agent.

2. The therapeutic combination of claim 1, wherein the I₁ agent is an I₁ agonist.

3. The therapeutic combination of claim 1, wherein the I₁ agent has a selectivity for the I₁ receptor over the alpha-2 (α2) adrenergic receptor of at least about 30:1.

4. The therapeutic combination of claim 1, wherein the I₁ agent is agmatine, BDBM50091347, clonidine, guanfacine, mCPP, memantine, moxonidine, naphazoline, oxymetazoline, rilmenidine, tetryzoline, tizanidine, or tolonidine, or a pharmaceutically acceptable salt thereof.

5. The therapeutic combination of claim 4, wherein the I₁ agent is clonidine, moxonidine, or rilmenidine, or a pharmaceutically acceptable salt thereof.

6. The therapeutic combination of claim 5, wherein the I₁ agent is clonidine, or a pharmaceutically acceptable salt thereof.

7. The therapeutic combination of claim 5, wherein the I₁ agent is moxonidine, or a pharmaceutically acceptable salt thereof.

8. The therapeutic combination of claim 5, wherein the I₁ agent is rilmenidine, or a pharmaceutically acceptable salt thereof.

9. The therapeutic combination of claim 1, wherein the 5-HT₇ agent is a 5-HT₇ agonist.

10. The therapeutic combination of claim 1, wherein the 5-HT₇ agent has a 5-HT₇ receptor Kᵢ of less than 10 nM.

11. The therapeutic combination of claim 1, wherein the 5-HT₇ agent is a tryptamine.

12. The therapeutic combination of claim 11, wherein the 5-HT₇ agent is 5-MeO-DMT or 5-MeO-MiPT, or a pharmaceutically acceptable salt thereof.

13. The therapeutic combination of claim 12, wherein the 5-HT₇ agent is 5-MeO-DMT, or a pharmaceutically acceptable salt thereof.

14. The therapeutic combination of claim 12, wherein the 5-HT₇ agent is 5-MeO-MiPT, or a pharmaceutically acceptable salt thereof.

15. The therapeutic combination of claim 1, wherein the therapeutic combination comprises clonidine, or a pharmaceutically acceptable salt thereof, and 5-MeO-DMT, or a pharmaceutically acceptable salt thereof.

16. The therapeutic combination of claim 1, wherein the therapeutic combination comprises moxonidine, or a pharmaceutically acceptable salt thereof, and 5-MeO-DMT, or a pharmaceutically acceptable salt thereof.

17. The therapeutic combination of claim 1, wherein the therapeutic combination comprises rilmenidine, or a pharmaceutically acceptable salt thereof, and 5-MeO-DMT, or a pharmaceutically acceptable salt thereof.

18. The therapeutic combination of claim 1, wherein the therapeutic combination comprises clonidine, or a pharmaceutically acceptable salt thereof, and 5-MeO-MiPT, or a pharmaceutically acceptable salt thereof.

19. The therapeutic combination of claim 1, wherein the therapeutic combination comprises moxonidine, or a pharmaceutically acceptable salt thereof, and 5-MeO-MiPT, or a pharmaceutically acceptable salt thereof.

20. The therapeutic combination of claim 1, wherein the therapeutic combination comprises rilmenidine, or a pharmaceutically acceptable salt thereof, and 5-MeO-MiPT, or a pharmaceutically acceptable salt thereof.

21. The therapeutic combination of any one of claims 1-20, further comprising a 5-HT₂ agent, a CB₁ agent, an additional I₁ agent, or an additional 5-HT₇ agent.

22. The therapeutic combination of any one of claims 1-20, further comprising a 5-HT₂ agent.

23. The therapeutic combination of claim 22, wherein the 5-HT₂ agent is a 5-HT₂ agonist.

24. The therapeutic combination of claim 23, wherein the 5-HT₂ agonist is 1-methyl psilocin, 2C-B-FLY, 5-MeO-AMT, AL-37350A, CP 809101, DALT, DOB, DOET, DOHx, MEM, CPP, NBOH-2C-CN, TCB-2, or WAY 161503, or a pharmaceutically acceptable salt thereof.

25. The therapeutic combination of any one of claims 1-20, further comprising a CB₁ agent.

26. The therapeutic combination of claim 25, wherein the CB₁ agent is a cannabinoid.

27. The therapeutic combination of claim 25, wherein the CB₁ agent is 2-AGE, A-834,735, ACEA, AZ-11713908, AZD-1940, CBN, CP-47497, CP55940, HU-210, JWH-007, JWH-051, JWH-146, JWH-176, JWH-200, JWH-398, Org 28611, rimonabant, THC, WIN 55,212-2, XLR11, or a pharmaceutically acceptable salt thereof.

28. The therapeutic combination of any one of claims 1-20, further comprising an additional I₁ agent.

29. The therapeutic combination of claim 28, wherein the additional I₁ agent is an I₁ agonist.

30. The therapeutic combination of claim 28, wherein the additional I₁ agent has a selectivity for the I₁ receptor over the alpha-2 (α2) adrenergic receptor of at least about 30:1.

31. The therapeutic combination of claim 28, wherein the additional I₁ agent is agmatine, BDBM50091347, clonidine, guanfacine, mCPP, MDMA, memantine, moxonidine, naphazoline, oxymetazoline, rilmenidine, tetryzoline, tizanidine, or tolonidine, or a pharmaceutically acceptable salt thereof.

32. The therapeutic combination of claim 31, wherein the additional I₁ agent is clonidine, moxonidine, or rilmenidine, or a pharmaceutically acceptable salt thereof.

33. The therapeutic combination of any one of claims 1-20, further comprising an additional 5-HT₇ agent.

34. The therapeutic combination of claim 33, wherein the additional 5-HT₇ agent is a 5-HT₇ agonist.

35. The therapeutic combination of claim 33, wherein the additional 5-HT₇ agent has a 5-HT₇ receptor Kᵢ of less than 10 nM.

36. The therapeutic combination of claim 33, wherein the additional 5-HT₇ agent is DMT, 6-F-DMT, 5-MeO-MiPT, LSD, 5-MeO-DMT, 5-MeO-TMT, cis-2a, DPT, 5-MeO-DiPT, psilocin, RR-2b, EMDT, lisuride, DiPT, SS-2c, TMA, 2C-B, 2C-E, or MDA, or pharmaceutically acceptable salt thereof.

37. The therapeutic combination of claim 36, wherein the additional 5-HT₇ agent is 5-MeO-DMT or 5-MeO-MiPT, or a pharmaceutically acceptable salt thereof.

38. The therapeutic combination of any one of claims 1-20, further comprising:
a. a 5-HT₂ agent and a CB₁ agent;
b. a 5-HT₂ agent and an additional I₁ agent;
c. a 5-HT₂ agent and an additional 5-HT₇ agent;
d. a 5-HT₂ agent and an additional 5-HT₂ agent;
e. a CB₁ agent and an additional I₁ agent;
f. a CB₁ agent and an additional 5-HT₇ agent; or
g. a CB₁ agent and an additional CB₁ agent.

39. The therapeutic combination of claim 38, wherein the 5-HT₂ agent is a 5-HT₂ agonist.

40. The therapeutic combination of claim 40, wherein the 5-HT₂ agonist is 1-methyl psilocin, 2C-B-FLY, 5-MeO-AMT, AL-37350A, CP 809101, DALT, DOB, DOET, DOHx, MEM, CPP, NBOH-2C-CN, TCB-2, or WAY 161503, or a pharmaceutically acceptable salt thereof.

41. The therapeutic combination of claim 38, wherein the additional 5-HT₂ agent is a 5-HT₂ agonist.

42. The therapeutic combination of claim 41, wherein the additional 5-HT₂ agonist is 1-methyl psilocin, 2C-B-FLY, 5-MeO-AMT, AL-37350A, CP 809101, DALT, DOB, DOET, DOHx, MEM, CPP, NBOH-2C-CN, TCB-2, or WAY 161503, or a pharmaceutically acceptable salt thereof.

43. The therapeutic combination of claim 38, wherein the CB₁ agent is a cannabinoid.

44. The therapeutic combination of claim 38, wherein the CB₁ agent is 2-AGE, A-834,735, ACEA, AZ-11713908, AZD-1940, CBN, CP-47497, CP55940, HU-210, JWH-007, JWH-051, JWH-146, JWH-176, JWH-200, JWH-398, Org 28611, rimonabant, THC, WIN 55,212-2, XLR11, or a pharmaceutically acceptable salt thereof.

45. The therapeutic combination of claim 38, wherein the additional CB₁ agent is a cannabinoid.

46. The therapeutic combination of claim 38, wherein the additional CB₁ agent is 2-AGE, A-834,735, ACEA, AZ-11713908, AZD-1940, CBN, CP-47497, CP55940, HU-210, JWH-007, JWH-051, JWH-146, JWH-176, JWH-200, JWH-398, Org 28611, rimonabant, THC, WIN 55,212-2, XLR11, or a pharmaceutically acceptable salt thereof.

47. The therapeutic combination of claim 38, wherein the additional I₁ agent is an I₁ agonist.

48. The therapeutic combination of claim 38, wherein the additional I₁ agent has a selectivity for the I₁ receptor over the alpha-2 (α2) adrenergic receptor of at least about 30:1.

49. The therapeutic combination of claim 38, wherein the additional I₁ agent is agmatine, BDBM50091347, clonidine, guanfacine, mCPP, MDMA, memantine, moxonidine, naphazoline, oxymetazoline, rilmenidine, tetryzoline, tizanidine, or tolonidine, or a pharmaceutically acceptable salt thereof.

50. The therapeutic combination of claim 79, wherein the additional I₁ agent is clonidine, moxonidine, or rilmenidine, or a pharmaceutically acceptable salt thereof.

51. The therapeutic combination of claim 38, wherein the additional 5-HT₇ agent has a 5-HT₇ receptor Kᵢ of less than 10 nM.

52. The therapeutic combination of claim 38, wherein the additional 5-HT₇ agent is DMT, 6-F-DMT, 5-MeO-MiPT, LSD, 5-MeO-DMT, 5-MeO-TMT, cis-2a, DPT, 5-MeO-DiPT, psilocin, RR-2b, EMDT, lisuride, DiPT, SS-2c, TMA, 2C-B, 2C-E, or MDA, or pharmaceutically acceptable salt thereof.

53. The therapeutic combination of claim 52, wherein the additional 5-HT₇ agent is 5-MeO-DMT or 5-MeO-MiPT, or a pharmaceutically acceptable salt thereof.

54. A pharmaceutical composition comprising the therapeutic combination of any one of claims 1-20, and a pharmaceutically acceptable carrier, diluent, or excipient.

55. The pharmaceutical composition of claim 54, wherein the composition is suitable for oral, buccal, sublingual, intranasal, ophthalmic, injectable, subcutaneous, intravenous, or transdermal administration.

56. The pharmaceutical composition of claim 54, wherein the composition is provided in unit dosage form.

57. The pharmaceutical composition of claim 56, wherein said unit dosage form is an immediate release, controlled release, sustained release, extended release, or modified release formulation.

58. A method of eliciting an entactogenic mindstate in a subject, comprising administering to the subject the therapeutic combination of any one of claims 1-20, or the pharmaceutical composition of claim 54.

59. The method of claim 58, wherein the agents of the therapeutic combination of any one of claims 1-20 or the pharmaceutical composition of claim 54 are administered simultaneously, separately, or sequentially.

60. The method of claim 59, wherein the agents are administered simultaneously.

61. The method of claim 58, wherein the agents are administered separately.

62. The method of claim 58, wherein the agents are administered sequentially.

63. The method of claim 62, wherein the I₁ agent is administered prior to the 5-HT₇ agent.

64. The method of claim 62, wherein when the therapeutic combination or composition comprises a CB₁ agent, the CB₁ agent is administered prior to the 5-HT₇ agent.

65. The method of claim 62, wherein when the therapeutic combination or composition comprises a CB₁ agent, the CB₁ agent is administered after the I₁ agent.

66. The method of claim 58, wherein the I₁ agent is administered orally.

67. The method of claim 58, wherein the 5-HT₇ agent is administered orally, sublingually, intranasally, or by inhalation.

68. The method of claim 58, wherein when the therapeutic combination or composition comprises a CB₁ agent, the CB₁ agent is administered orally, sublingually, or by inhalation.

69. The method of claim 58, wherein the I₁ agent is administered at a dose of between about 0.1 mg and 1.0 mg.

70. The method of claim 58, wherein the 5-HT₇ agent is administered at a dose of between about 2 mg and 20 mg.

71. The method of claim 58, further comprising administering a therapeutically effective amount of an additional active agent.

72. The method of claim 71, wherein the additional therapeutic agent is an amino acid, antioxidant, anti-inflammatory agent, analgesic, antineuropathic or antinociceptive agent, antimigraine agent, anxiolytic, antidepressant, antipsychotic, anti-PTSD agent, cannabinoid, dissociative, immunostimulant, anti-cancer agent, antiemetic, orexigenic, antiulcer agent, antihistamine, anti hypertensive, anticonvulsant, antiepileptic, bronchodilator, neuroprotectant, entactogen or empathogen, entheogen, psychedelic, monoamine oxidase inhibitor, tryptamine, terpene, phenethylamine, sedative, serotonergic agent, stimulant, vitamin, SSRI, SNRI, NDRI, TCA, or benzodiazepine.

73. The method of claim 58, wherein the entactogenic mindstate is a similar entactogenic mindstate to MDMA or another known entactogen, wherein the entactogenic mindstate is determined by brain imaging or by measuring the level of one or more neurotransmitters in the subject.

74. The method of claim 58, wherein the entactogenic mindstate is a greater entactogenic mindstate to MDMA or another known entactogen, wherein the entactogenic mindstate is determined by brain imaging or by measuring the level of one or more neurotransmitters in the subject.

75. The method of claim 58, wherein the greater entactogenic mindstate is an expanded entactogenic mindstate or an enhanced entactogenic mindstate, when compared to MDMA or another known entactogen, wherein the entactogenic mindstate is determined by brain imaging or by measuring the level of one or more neurotransmitters in the subject.

76. The method of any one of claims 72-75, wherein the neurotransmitter is one or more of serotonin, dopamine, norepinephrine, oxytocin, or cortisol.

77. The method of claim 58, wherein the method does not produce an adverse effect of MDMA or another known entactogen, or produces a reduced adverse effect of MDMA or another known entactogen.

78. The method of claim 77, wherein the adverse effect is a neurotoxic effect.

79. The method of claim 78, wherein an absence or reduction of a neurotoxic effect is determined by tests and procedures that are in silico, in vitro, or in vivo.

80. The method of claim 79, wherein the absence or reduction of a neurotoxic effect is determined by measuring one or more of: a) at least one toxic metabolite of MDMA or at least one toxic metabolite of another entactogen; b) oxidative stress and dopamine-based quinones; c) mitochondrial dysfunction; and d) activation of glial cells.

81. The method of claim 80, wherein the reduction of a neurotoxic effect is at least 5%, 10%, 25%, 50%, 75%, 100%, 150%, or 200% relative to one or more comparators.

82. A method for modulating neurotransmission in a mammal, comprising administering to the mammal the therapeutic combination of any one of claims 1-20, or the pharmaceutical composition of claim 54.

83. The method of claim 82, wherein the neurotransmission is mediated by an I₁ receptor, a 5-HT₂ receptor, a 5-HT₇ receptor, or a CB₁ receptor.

84. The method of claim 83, wherein the mammal is a human.

85. A method of treating a medical condition in a human in need of such treatment, comprising administering to the human the therapeutic combination of any one of claims 1-20, or the pharmaceutical composition of claim 54.

86. The method of claim 85, wherein the medical condition is a disorder linked to dysregulation or inadequate functioning of neurotransmission.

87. The method of claim 86, wherein the disorder is linked to dysregulation or inadequate functioning of neurotransmission mediated by an I₁ receptor, a 5-HT₂ receptor, a 5-HT₇ receptor, or a CB₁ receptor.

88. The method of claim 85, wherein the medical condition is a CNS disorder.

89. The method of claim 88, wherein the CNS disorder is any of: post-traumatic stress disorder (PTSD), adjustment disorder, affective disorder, depression, atypical depression, postpartum depression, catatonic depression, a depressive disorder due to a medical condition, premenstrual dysphoric disorder, seasonal affective disorder, dysthymia, anxiety, phobia disorders, binge disorders, body dysmorphic disorder, alcohol or drug abuse or dependence disorders, substance-related disorders, substance-induced mood disorder, a mood disorder related to another health condition, disruptive behavior disorders, eating disorders, impulse control disorders, obsessive compulsive disorder (OCD), attention deficit hyperactivity disorder (ADHD), personality disorders, attachment disorders, dissociative disorders, Alzheimer's disease, ataxia, Huntington's disease, Parkinson's disease, motor neuron disease, multiple system atrophy, progressive supranuclear palsy, migraines, cluster headaches, short-lasting unilateral neuralgiform headaches, fibromyalgia, traumatic brain injury, and mild traumatic brain injury (mTBI).

90. The method of claim 88, wherein the CNS disorder is a disorder related to rigid modes of thinking.

91. The method of claim 90, wherein the disorder related to rigid modes of thinking is anxiety, depression, addiction, an eating disorder, an alcohol or drug abuse or dependence disorder, OCD, or PTSD.

92. The method of claim 89 or 91, wherein depression is MDD or TRD.

93. The method of claim 89 or 91, wherein anxiety is GAD.

94. A method of improving mental health or functioning in a human, the method comprising identifying a human in need of said improving, and administering to the human the therapeutic combination of any one of claims 1-20, or the pharmaceutical composition of claim 54.

95. The method of claim 94, wherein the improvement in mental health or functioning is a reduction of neuroticism or psychological defensiveness, an increase in creativity or openness to experience, an increase in decision-making ability, an increase in ability to fall or stay asleep, an increase in feelings of wellness or satisfaction, an increase in the ability to forgive oneself or another, an increase in the ability to experience or contemplate pain, including mental pain, or an increase in the ability to “touch within,” including in and during everyday life and activities.

96. The method of claim 95, wherein the increase in creativity is an increase in deliberate cognitive creativity, deliberate emotional creativity, spontaneous cognitive creativity, or spontaneous emotional creativity.

97. A method of reducing the symptoms of a CNS disorder in a human, the method comprising identifying a human in need of said reducing, and administering to the human the therapeutic combination of any one of claims 1-20, or the pharmaceutical composition of claim 54.

98. A therapeutic combination for eliciting an entactogenic mindstate, comprising:
a. the therapeutic combination of any one of claims 1-53; or
b. an I₁ agent and one or more of a 5-HT₂ agent, a 5-HT₇ agent, and a CB₁ agent.

99. A pharmaceutical composition comprising the therapeutic combination of any of the foregoing claims, and a pharmaceutically acceptable carrier, diluent, or excipient.

100. The pharmaceutical composition of claim 99, wherein the composition is suitable for oral, buccal, sublingual, intranasal, ophthalmic, injectable, subcutaneous, intravenous, or transdermal administration.

101. The pharmaceutical composition of claim 99 or 100, wherein the composition is provided in unit dosage form.

102. The pharmaceutical composition of claim 101, wherein said unit dosage form is an immediate release, controlled release, sustained release, extended release, or modified release formulation.

103. A method of eliciting an entactogenic mindstate in a subject, comprising administering to the subject the therapeutic combination or pharmaceutical composition of any of the foregoing claims.

104. A method for modulating neurotransmission in a mammal, comprising administering to the mammal the therapeutic combination or pharmaceutical composition of any of the foregoing claims.

105. A method of treating a medical condition in a human in need of such treatment, comprising administering to the human the therapeutic combination or pharmaceutical composition of any of the foregoing claims.

106. A method of improving mental health or functioning in a human, the method comprising identifying a human in need of said improving, and administering to the human the therapeutic combination or pharmaceutical composition of any of the foregoing claims.

107. A method of reducing the symptoms of a CNS disorder in a human, the method comprising identifying a human in need of said reducing, and administering to the human the therapeutic combination or pharmaceutical composition of any of the foregoing claims.

108. The method of any of the foregoing claims, wherein the pharmaceutical composition or therapeutic combination is administered together with psychotherapy.

109. The method of any of the foregoing claims, wherein the pharmaceutical composition or therapeutic combination is administered together with psychological support.

110. The method of any of the foregoing claims, wherein the pharmaceutical composition or therapeutic combination is administered together with patient monitoring.

111. The method of any of the foregoing claims, wherein the pharmaceutical composition or therapeutic combination is administered together with the performance of a therapeutically beneficial activity by the patient.

112. The method of any of the foregoing claims, wherein the pharmaceutical composition or therapeutic combination is administered without psychotherapy or psychological support.
